(19) United States
(12) Patent Application Publication
(10) Pub. No.: US 2017/0266266 A1
(43) Pub. Date: Sep. 21, 2017

(54) Functions of 55 Newfound Proteins and Their Medicinal Application in the Treatment and Prevention of Disease

(71) Applicant: Kieu Hoang, Agoura Hills, CA (US)

(72) Inventor: Kieu Hoang, Agoura Hills, CA (US)

(21) Appl. No.: 14/471,466

(22) Filed: Aug. 28, 2014

Related U.S. Application Data
(63) Continuation-in-part of application No. 13/756,478, filed on Jan. 31, 2013, now abandoned.
(60) Provisional application No. 61/926,286, filed on Jan. 11, 2014.
(60) Provisional application No. 61/926,287, filed on Jan. 11, 2014.
(60) Provisional application No. 61/593,164, filed on Jan. 31, 2012.
(60) Provisional application No. 61/593,196, filed on Jan. 31, 2012.
(60) Provisional application No. 61/648,281, filed on May 17, 2012.
(60) Provisional application No. 61/692,273, filed on Aug. 23, 2012.
(60) Provisional application No. 61/710,930, filed on Oct. 8, 2012.

(51) Int. Cl. A61K 38/48; A61K 38/44; A61K 38/17; A61K 9/00; A61K 38/16; A61K 38/40; A61K 35/16; A61K 38/36; A61K 38/38

(52) U.S. Cl. CPC A61K 38/4846; A61K 38/4833; A61K 38/36; A61K 38/44; A61K 38/1748; A61K 38/385; A61K 38/1722; A61K 38/16; A61K 38/40; A61K 38/17; A61K 38/363; A61K 35/16; A61K 38/1709; C12Y 304/21005; C12Y 304/21021; C12Y 304/21006; C12Y 116/03001; C12Y 304/21022; C12Y 105/01005; A61K 9/0019

(57) ABSTRACT

The invention relates to 55 newly discovered proteins, which are present in isolated purified protein complexes, derived medicinal products, recombinant DNA, engineered DNA, cDNA, monoclonal and natural products or synthesized products as part of nutrition, food, and/or supplemental products and their applications.

### CROSS REFERENCE TO RELATED APPLICATIONS

This application claims priority under 35 USC 119(e) to provisional applications Nos. 61/926,286 and 61/926,287, both filed on Jan. 11, 2014, which are incorporated herein by reference in their entireties. This application is also a continuation in part of non-provisional application Ser. No. 13/756,478, filed on Jan. 31, 2013, which claims priority to provisional application No. 61/593,164, filed on Jan. 31, 2012, provisional application No. 61/593,183, filed on Jan. 31, 2012, provisional application No. 61/593,196, filed Jan. 31, 2012, provisional application No. 61/648,281, filed on May 17, 2012, provisional application No. 61/692,273, filed on Aug. 23, 2012 and provisional application No. 61/710,930, filed on Oct. 8, 2012, all of which are hereby incorporated herein by reference in their entireties.

### FIELD OF THE INVENTION

55 novel proteins have been identified in various biological constructs and have been sequenced, and tested for their medicinal application in the treatment and prevention of disease.

### BACKGROUND OF THE INVENTION

Cells are the smallest component in an organism and are responsible for the production of proteins that can either cause harm to the organism or prevent and treat disease and infection in the organism. Since the discovery of cells as a basic building block of human life, scientists have been studying them in live (in vivo) and cultured mediums (in vitro) to try and discover the key to disease prevention.

As determined by the World Health Organization in 2014, cancer accounts for 8.2 million deaths per year and 14.6% of all deaths worldwide. There are various methods and drugs used in the treatment of cancer, including surgery excision, chemotherapy, radiotherapy, hormonal medications, and for sexual tract cancers the removal of sex organs that produce tumor cell-stimulating hormones, e.g. ER/PR positive cancers. While effective, many of these therapies contribute to side effects that can be almost as debilitating as the original disease. For example, it is extremely common to treat breast or ovarian cancers with surgical removal of the tumor(s) in addition to chemotherapy, which may produce early onset menopause and require long-term drug therapy to offset the premature aging of the body. This premature aging process can lead to osteopenia, osteoporosis, hot flashes, and vaginal dryness—and subsequent long-term treatment with expensive bisphosphonates and hormonal drugs to offset its effects. It is therefore desirable to develop cancer treatments that can be used alone or in combination with other cancer therapies, which have little or no side effects, yet are effective in treating or preventing the progression of the disease.

While cancer is perhaps the most nefarious affliction affecting human health, other diseases and infections that have been identified and studied in detail by scientists remain prevalent and without cure in society. The methods and drugs that have been developed to treat those diseases and infections often come with their own repercussions and adverse side effects. Examples of some of the most widely studied diseases and viruses that require better treatment options include, HIV, hepatitis, diabetes, atherosclerosis and related cardiovascular diseases, influenza, and Parkinson's disease.

Therefore, KH cells and proteins have been discovered, isolated, and purified in combination for treating a wide variety of diseases, infections, and other physical conditions and disorders, without many of the repercussions and adverse side effects of previously discovered drugs and methods of treatment.

### SUMMARY OF THE INVENTION

55 proteins have been discovered, isolated, and purified for use in various constructs helpful in treating and preventing a wide variety of diseases, infections, disorders, and afflictions which adversely affect health.

### BRIEF DESCRIPTION OF THE DRAWINGS

FIG. 1 shows graphical representation of prism files containing dose-dependent curves.

FIG. 2 is a graph depicting the inhibition rate of HIV-1 treated with various plasma products.

FIG. 3 is a graph depicting the inhibition rate of HIV-1 compared with the dilution of various plasma products.

FIG. 4 is a graph of OD 450 and dilution of various plasma products used in treating HIV-1.

FIGS. 5 and 6 are graphs showing the dose dependent curves of various plasma products used in the treatment of HVC.

FIGS. 7-15 are graphs comparing FSC/SSC on FACS.

FIGS. 16-22 are graphs comparing human T/B cells on FACS.

FIGS. 23-30A are graphs comparing human granulocytes on FACS.

FIG. 30B is a graph comparing human NK cells on FACS.

FIG. 31A is a graph depicting AFOD 1 vs. human insulin results.

FIG. 31B is a graph depicting AFOD RAAS 101 vs. human insulin results.

FIG. 31C is a graph depicting AFOD RAAS 103 vs. human insulin results.

FIG. 31D is a graph depicting AFOD RAAS 107 vs. human insulin results.

FIG. 31E is a graph depicting AFOD RAAS 108 vs. human insulin results.

FIG. 31F is a graph depicting AFOD RAAS 109 vs. human insulin results.

FIG. 31G is a graph depicting AFOD RAAS 110 vs. human insulin results.

FIG. 31H is a graph depicting AFOD RAAS 120 vs. human insulin results.

FIG. 31I is a graph depicting AFOD RAAS 121 vs. human insulin results.

FIG. 31J is a graph depicting AFOD RAAS KH vs. human insulin results.

FIG. 31K is a graph depicting AFCC KH1 vs. human insulin results.

FIG. 31L is a graph depicting KH105(1) vs. human insulin results.

FIG. 31M is a graph depicting KH105(2) vs. human insulin results.

FIG. 31N shows dose response graphs of glucose uptake for AFOD RAAS 107 and AFOD KH_N1.

FIG. 31O shows dose response graphs of glucose uptake for AFOD RAAS 107 and AFOD KH_N2.

FIG. 31P shows dose response graphs of glucose uptake for AFOD RAAS 107 and AFOD KH_N3.

FIG. 31Q shows dose response graphs of glucose uptake for AFOD 1 and AFOD RAAS 109_N1.

FIG. 31R shows dose response graphs of glucose uptake for AFOD 1 and AFOD RAAS 109_N2.

FIGS. 31S-31Z show additional response graphs of glucose uptake.

FIGS. 32-36 are pictures of the aorta of mice in the study of APOAI protein in preventing atherosclerosis and related cardiovascular diseases. FIG. 32 is the vehicle control group. FIG. 33 is the low dose group. FIG. 34 is the medium dose group. FIG. 35 is the high dose group. FIG. 36E is the positive control (Lipitor) group.

FIGS. 36A-36D are pictures of organs of mice in the study of APOAI protein in preventing atherosclerosis and related cardiovascular diseases. FIG. 36A shows liver fatty change of control and treated animals. FIG. 36B shows fat deposits on the heart of control and treated animals. FIG. 36C shows atherosclerosis on control and treated animals, first view. FIG. 36D shows atherosclerosis change on control and treated animals, second view.

FIGS. 37-38 are pictures of dissected mice in the study: pre-clinical animal test of Apo-AI—for the antiatherogenic and cholesterol-lowering properties. FIG. 37 shows the fatty streak lesions and liver change of a animals fed with a high fat diet and sacrificed on week 10. FIG. 38 shows the plaque area change with normal diet after 10 weeks with a high fat diet in the control group—animals which were given a normal diet after establishing fatty streak lesions in aortas.

FIG. 39 is a graph showing change of weight and plasma lipid concentrations compared between APOAI treated and control animals.

FIG. 40 is a picture of a normal rabbit aorta without fatty streak lesion.

FIG. 41 is a picture of the area of fatty streak lesion in the aorta from the APOAI group 1.

FIG. 42 is a picture of the area of fatty streak lesion in the aorta from the APOAI group 2.

FIG. 43 is a graph comparing number of days treated with AFCC vs. bodyweight.

FIG. 44 is a graph comparing days post infection vs. survival rate.

FIG. 45 is a graph comparing days post infection vs. bodyweight.

FIG. 46 is a graph comparing days post infection vs. bodyweight change.

FIG. 47 is a graph comparing days post infection vs. bodyweight change.

FIG. 48 is a graph comparing days post infection vs. survival distribution function expressed as a percentage.

FIG. 49 is a graph comparing days before and after infection vs. bodyweight change.

FIG. 50 is a graph comparing HBV copies/ul plasma vs. days after infection for various test groups.

FIG. 51 is a graph of the effect of prophylactic treatment or therapeutic treatment of RAAS 8 on ETV on the HBsAg in mouse blood.

FIG. 52 is a graph of the effect of prophylactic treatment or therapeutic treatment of RAAS 8 on ETV on the intermediate HBV replication in the mouse livers by qPCR.

FIG. 53 is a southern blot determination of intermediate HBV DNA in mouse livers.

FIG. 54 is a graph depicting the bodyweight of mice treated with vehicle or indicated compounds during the course of experiment.

FIG. 55 is a graph depicting the efficacy of therapeutic treatment of RAAS 105 on in vivo HBV replication in plasma in mouse HDI model.

FIG. 56 is a graph depicting the efficacy of prophylactic treatment of RAAS 105 on in vivo HBV replication in plasma in mouse HDI model.

FIG. 57 is a graph depicting the effect of therapeutic treatment of RAAS 105 on the HBsAg in mouse plasma.

FIG. 58 is a graph depicting the effect of prophylactic treatment of RAAS 105 on the HBsAg in mouse plasma.

FIG. 59 is a graph depicting the effect of therapeutic treatment of RAAS 105 on the intermediate HBV replication in the mouse livers by qPCR.

FIG. 60 is a graph depicting the effect of prophylactic treatment of RAAS 105 on the intermediate HBV replication in the mouse livers by qPCR.

FIG. 61 is a graph depicting the body weights of mice in all groups treated with vehicle or indicated compounds during the course of the experiment “Efficacy of A Human Plasma Derived Protein AFOD RAAS 105 in Inhibition of the HBV Replication in the Mouse Hydrodynamic Injection Model” (n=10).

FIG. 62 is a graph depicting percentages of T and B lymphocytes in peripheral blood, with and without therapeutic RAAS 105 treatment.

FIG. 63 is a graph depicting percentages of T and B lymphocytes in peripheral blood, with further analysis done on CD4 and CD8 T cell lineages, with and without therapeutic RAAS 105 treatment.

FIG. 64 is a graph depicting percentages of CD4 and CD8 T cells in peripheral blood, with and without therapeutic RAAS 105 treatment.

FIG. 65 is a graph depicting percentages of CD4 and CD8 T cells in peripheral blood, with further analysis done on the percentages of CD11c⁺ dendritic cells (DC) and Gr-1⁺ granulocytes.

FIG. 66 is graphs depicting percentages of dendritic cells and granulocytes in peripheral blood, with and without therapeutic RAAS 105 treatment.

FIG. 67 is graph showing another representation of Gr-1 vs. CD 11c cells, with and without therapeutic RAAS 105 treatment.

FIG. 68 is a graph depicting the percentage of monocytes in peripheral blood, with and without therapeutic RAAS 105 treatment.

FIG. 69 is a graph showing another representation of monocytes in peripheral blood, with and without therapeutic RAAS 105 treatment.

FIG. 70 is graphs depicting percentages of T and B lymphocytes in the spleen, with and without therapeutic RAAS 105 treatment.

FIG. 71 is a graph showing another representation of T and B lymphocytes in the spleen, with and without therapeutic RAAS 105 treatment.

FIG. 72 is graphs depicting percentages of CD4 and CD8 T cells in the spleen, with and without therapeutic RAAS 105 treatment.

FIG. 73 is a graph showing another representation of CD4 and CD8 T cells in the spleen, with CD3 T cells being gated, with and without therapeutic RAAS 105 treatment.

FIG. 74 is graphs depicting T cell subset percentages in the spleen, with and without therapeutic RAAS 105 treatment.

FIG. 75 is a graph of CD4 T cell subset percentages in the spleen, with and without therapeutic RAAS 105 treatment.

FIG. 76 is graphs depicting T cell subset percentages in the spleen, with and without therapeutic RAAS 105 treatment.

FIG. 77 is a graph of CD8 T cell subset percentages in the spleen, with and without therapeutic RAAS 105 treatment.

FIG. 78 is a graph depicting percentages of regulatory T cells in the spleen, with and without therapeutic RAAS 105 treatment.

FIG. 79 is another graphical representation of percentages of regulatory T cells in the spleen, with and without therapeutic RAAS 105 treatment.

FIG. 80 is graphs depicting percentages of mDc and pDcs in the spleen, with and without therapeutic RAAS 105 treatment.

FIG. 81 is another graphical representation of mDC and pDcs in the spleen, with and without therapeutic RAAS 105 treatment.

FIG. 82 is graphs depicting percentages of macrophages and granulocytes in the spleen, with and without therapeutic RAAS 105 treatment.

FIG. 83 is another graphical representation of percentages of macrophages and granulocytes in the spleen, with and without therapeutic RAAS 105 treatment.

FIG. 84 is a graph depicting percentages of T cells in the lymph nodes, with and without therapeutic RAAS 105 treatment.

FIG. 85 is graphs showing percentages of CD3 T cells in the lymph nodes, with and without therapeutic RAAS 105 treatment.

FIG. 86 is graphs depicting percentages of CD4 and CD8 T cells in the lymph nodes, with and without therapeutic RAAS 105 treatment.

FIG. 87 is another graphical representation of CD4 and CD8 T cells in the lymph nodes, with and without therapeutic RAAS 105 treatment.

FIG. 88 is graphs depicting CD4 T cell subset percentages in the lymph nodes, with and without therapeutic RAAS 105 treatment.

FIG. 89 is another graphical representation of CD4 T cell subset percentages in the lymph nodes, with and without therapeutic RAAS 105 treatment.

FIG. 90 is graphs depicting CD8 T cell subset percentages in the lymph nodes, with and without therapeutic RAAS 105 treatment.

FIG. 91 is another graphical representation of CD8 T cell subset percentages in the lymph nodes, with and without therapeutic RAAS 105 treatment.

FIG. 92 is a graph depicting percentages of Foxp3 regulatory T cells in the lymph nodes, with and without therapeutic RAAS 105 treatment.

FIG. 93 is another graphical representation of Foxp3 regulatory T cells in the lymph nodes, with and without therapeutic RAAS 105 treatment.

FIG. 94 is a graph depicting percentages of DCs in the lymph nodes, with and without therapeutic RAAS 105 treatment.

FIG. 95 is another graphical representation of percentages of DCs in the lymph nodes, with and without therapeutic RAAS 105 treatment.

FIG. 96 is graphs depicting percentages of macrophages and granulocytes in the lymph nodes, with and without therapeutic RAAS 105 treatment.

FIG. 97 is another graphical representation of percentages of macrophages and granulocytes in the lymph nodes, with and without therapeutic RAAS 105 treatment.

FIG. 98 is graphs depicting T and B lymphocytes in peripheral blood, with and without prophylactic RAAS 105 treatment.

FIG. 99 is another graphical representation of T and B cells in peripheral blood, with and without prophylactic RAAS 105 treatment.

FIG. 100 is graphs depicting percentages of CD4 and CD 8 T cells in peripheral blood, with and without prophylactic RAAS 105 treatment.

FIG. 101 is another graphical representation of CD4 and CD 8 T cells in peripheral blood, with and without prophylactic RAAS 105 treatment.

FIG. 102 is graphs depicting percentages of dendritic cells and granulocytes in peripheral blood, with and without prophylactic RAAS 105 treatment.

FIG. 103 is another graphical representation of dendritic cells and granulocytes in peripheral blood, with and without prophylactic RAAS 105 treatment.

FIG. 104 is a graph depicting percentages of monocytes in peripheral blood, with and without prophylactic RAAS 105 treatment.

FIG. 105 is another graphical representation of percentages of monocytes in peripheral blood, with and without prophylactic RAAS 105 treatment.

FIG. 106 is graphs depicting percentages of T and B lymphocytes in the spleen, with and without prophylactic RAAS 105 treatment.

FIG. 107 is another graphical representation of percentages of T and B lymphocytes in the spleen, with and without prophylactic RAAS 105 treatment.

FIG. 108 is graphs depicting percentages of CD4 and CD8 T cells in the spleen, with and without prophylactic RAAS 105 treatment.

FIG. 109 is another graphical representation of percentages of CD4 and CD8 T cells in the spleen, with and without prophylactic RAAS 105 treatment.

FIG. 110 is graphs depicting subset percentages of T cells in the spleen, with and without prophylactic RAAS 105 treatment.

FIG. 111 is another graphical representation of subset percentages of T cells in the spleen, with and without prophylactic RAAS 105 treatment.

FIG. 112 is graphs depicting subset percentages of T cells in the spleen, with and without prophylactic RAAS 105 treatment.

FIG. 113 is another graphical representation of subset percentages of T cells in the spleen, with and without prophylactic RAAS 105 treatment.

FIG. 114 is a graph depicting Foxp3 regulator T cells in the spleen, with and without prophylactic RAAS 105 treatment.

FIG. 115 is another graphical representation of Foxp3 regulator T cells in the spleen, with and without prophylactic RAAS 105 treatment.

FIG. 116 is graphs depicting percentages of pDCs and mDCs in the spleen, with and without prophylactic RAAS 105 treatment.

FIG. 117 is another graphical representation of percentages of pDCs and mDCs in the spleen, with and without prophylactic RAAS 105 treatment.

FIG. 118 is graphs depicting percentages of macrophages and granulocytes in the spleen, with and without prophylactic RAAS 105 treatment.

FIG. 119 is another graphical representation of percentages of macrophages and granulocytes in the spleen, with and without prophylactic RAAS 105 treatment.

FIG. 120 is a graph depicting percentages of T cells in the lymph nodes, with and without prophylactic RAAS 105 treatment.

FIG. 121 is another graphical representation of percentages of CD3 T cells in the lymph nodes, with and without prophylactic RAAS 105 treatment.

FIG. 122 is graphs depicting percentages of CD4 and CD8 T cells in the lymph nodes, with and without prophylactic RAAS 105 treatment.

FIG. 123 is another graphical representation of percentages of CD4 and CD8 T cells in the lymph nodes, with and without prophylactic RAAS 105 treatment.

FIG. 124 is graphs depicting T cell subset percentages in the lymph nodes, with and without prophylactic RAAS 105 treatment.

FIG. 125 is another graphical representation of T cell subset percentages in the lymph nodes, with and without prophylactic RAAS 105 treatment.

FIG. 126 is graphs depicting T cell subset percentages in the lymph nodes, with and without prophylactic RAAS 105 treatment.

FIG. 127 is another graphical representation of T cell subset percentages in the lymph nodes, with and without prophylactic RAAS 105 treatment.

FIG. 128 is a graph depicting percentages of Foxp3 regulatory T cells in the lymph nodes, with and without prophylactic RAAS 105 treatment.

FIG. 129 is another graphical representation of Foxp3 regulatory T cells in the lymph nodes, with and without prophylactic RAAS 105 treatment.

FIG. 130 is a graph depicting percentages of DCs in the lymph nodes, with and without prophylactic RAAS 105 treatment.

FIG. 131 is another graphical representation of percentages of DCs in the lymph nodes, with and without prophylactic RAAS 105 treatment.

FIG. 132 is graphs depicting percentages of macrophages and granulocytes in the lymph nodes, with and without prophylactic RAAS 105 treatment.

FIG. 133 is another graphical representation of percentages of macrophages and granulocytes in the lymph nodes, with and without prophylactic RAAS 105 treatment.

FIG. 134 is graphs depicting the effects of AFOD KH, AFOD 103, AFOD 107, AFOD 108, and AFOD 1 on bodyweight (A) and bodyweight change (B).

FIG. 135 is graphs depicting the effects of AFCC KH, AFOD 101 and AFOD 102 on bodyweight (A) and bodyweight change (B).

FIG. 136 is graphs depicting the effects of AFOD KH, AFOD 103, AFOD 107, AFOD 108, and AFOD 1 on delta paw volume (A) and AUC of paw swelling (B).

FIG. 137 is graphs depicting the effects of AFCC KH, AFOD 101, and AFOD 102 on delta paw volume (A) and AUC of paw swelling (B).

FIG. 138 is a graph depicting the effects of AFOD KH, AFOD 103, AFOD 107, AFOD 108, and AFOD 1 on arthritic score.

FIG. 139 is a graph depicting the effects of AFCC KH, AFOD 101 and AFOD 102 on arthritic score.

FIG. 140 is a graph depicting the effects of AFOD KH, AFOD 103, AFOD 107, AFOD 108, and AFOD 1 on the incidence rate of arthritis.

FIG. 141 is a graph depicting the effects of AFCC KH, AFOD 101, and AFOD 102 on the incidence rate of arthritis.

FIG. 142 is a graph depicting the effect of various doses of APOA 1 on bodyweight.

FIG. 143 is a graph depicting the effect of HFD on the lipid profile in ApoE mice.

FIG. 144 is a graph depicting the effect of various doses of APOA 1 on plasma TC.

FIG. 145 is a graph depicting the net change of plasma TC with various doses of APOA 1.

FIG. 146 is a graph depicting the effect of various doses of APOA 1 on plasma triglycerides.

FIG. 147 is a graph depicting the effect of various doses of APOA 1 on plasma HDL levels.

FIG. 148 is a graph depicting the effect of various doses of APOA 1 on the net change of plasma LDL levels.

FIG. 149 is a graph depicting the effect of various doses of APOA 1 on plasma LDL levels.

FIG. 150 is a graph depicting the effect of various doses of APOA 1 on the net change of plasma HDL levels.

FIG. 151 is pictures of the effect of APOA 1 on the atherosclerosis plaque lesion of a mouse.

FIG. 152 is pictures of the effect of APOA 1 on the atherosclerosis plaque lesion of a mouse.

FIG. 153 is a graph depicting the percentage of plaque area in the total inner vascular area of mice treated with various doses of APOA 1.

FIG. 154 is a picture of the arterial arch area of a mouse.

FIG. 155 is a graph depicting the percent of root plaque area in the arterial arch area of mice treated with various doses of APOA 1.

FIG. 156 is a picture of the area analyzed of the root to the right renal artery.

FIG. 157 is a graph depicting the percentage of plaque area from the root to the right renal artery of mice treated with various doses of APOA 1.

FIG. 158 is a graph of the effect of various doses of APOA 1 on liver weight.

FIG. 159 is a graph of the effect of various doses of APOA 1 on liver/body weight.

FIG. 160 is a graph comparing percentages of plaque area of mice fed high fat diets for 4, 19, and 27 weeks, treated with various doses of APOA 1.

FIG. 161 is a graph comparing TC levels of mice fed high fat dies for 4, 19, and 27 weeks, treated with various vehicles.

FIG. 162 is a graph comparing LDL levels of mice fed high fat diets for 4, 19, and 27 weeks, treated with various vehicles.

FIG. 163 is another graphical representation comparing LDL levels of mice fed high fat dies for 4, 19, and 27 weeks, treated with various vehicles.

FIG. 164 is pictures of aorta plaque lesions after 16 weeks of treatment with various doses of APOA 1.

FIG. 165 is a graph depicting the anti-colo-rectal tumor efficacy of high concentrated fibrinogen enriched a1at thrombin and AFOD at various doses in PDX model CO-04-0002.

FIG. 166 is a graph depicting the anti-colo-rectal tumor efficacy of high concentrated fibrinogen enriched a1at thrombin and AFOD at various doses in PDX model CO-04-0002 and CO-04-0001.

FIG. 167 is a picture of colo-rectal derived tumors dissected from the abdominal cavity of mice.

FIG. 168 is a graph depicting the relative change of bodyweight of mice treated with high concentrated fibrinogen enriched a1at thrombin and AFOD at various doses.

FIG. 169 is a graph depicting the anti-lung-derived tumor efficacy of high concentrated fibrinogen enriched a1at thrombin and AFOD at various doses in PDX model LU-01-0032.

FIG. 170 is a picture of lung-derived tumors dissected from the abdominal cavity of mice.

FIG. 171 is a graph depicting ratios of mice with palpable lung cancer derived tumors observed in mice treated with high concentrated fibrinogen enriched a1at thrombin and AFOD at various doses.

FIG. 172 is a graph depicting the relative change of bodyweight of mice treated with high concentrated fibrinogen enriched a1at thrombin and AFOD at various doses.

FIG. 173-182 are ovarian cancer survival curve graphs of mice treated with AFOD RAAS 1, AFOD RAAS 104, AFOD RAAS 108, AFOD RAAS 109/121, AFOD RAAS 110, AFOD 113, AFOD RAAS 114, AFOD RAAS 120 and AFFC RAAS 1, AFCC RAAS 2, and positive and vehicle, respectively.

FIG. 183 is pictures of dissected mice in the ovarian cancer vehicle group vs. AFOD KH1 treatment group.

FIG. 184 is pictures of dissected mice in the ovarian cancer AFOD KH1 group.

FIG. 185 is pictures of dissected mice in the ovarian cancer positive control group.

FIG. 186 is pictures of dissected mice in the ovarian cancer AFOD RAAS 1 group.

FIG. 187 is pictures of dissected mice in the ovarian cancer AFOD RAAS 104 group.

FIG. 188 is pictures of dissected mice in the ovarian cancer AFOD RAAS 108 group.

FIG. 189 is pictures of dissected mice in the ovarian cancer AFOD RAAS 109/121 group.

FIG. 190 is pictures of dissected mice in the ovarian cancer AFOD RAAS 110 group.

FIG. 191 is pictures of dissected mice in the ovarian cancer AFOD RAAS 113 group.

FIG. 192 is pictures of dissected mice in the ovarian cancer AFOD RAAS 114 group.

FIG. 193 is pictures of dissected mice in the ovarian cancer AFCC RAAS 1 group.

FIG. 194 is pictures of dissected mice in the ovarian cancer AFCC RAAS 2 group.

FIG. 195 is pictures of dissected mice in the ovarian cancer AFOD RAAS 120 group.

FIG. 196 is a picture of a mouse implanted with MDA-MB-231-Luc tumor cells.

FIG. 197 is a picture of one of the 10 nude mice with MDA-MB-231-Luc tumor cells used in the re-implantation study, with dissection over tumor area revealed.

FIG. 198 is a picture of a mouse implanted with MDA-MB-231-Luc tumor cells.

FIG. 199 is a graph depicting tumor volume vs. days post implantation for mice implanted with MDA-MB-231-Luc tumor cells.

FIG. 200 is pictures of a mouse implanted with MDA-MB-231-Luc tumor cells with tumor site dissected.

FIG. 201 is pictures of the re-implantation of tumor tissues in nude mice.

FIG. 202 is a raw data of tumor growth in mice treated with AFCC prior to re-implantation

FIG. 203 is a graph showing 5 groups of nude mice after tumor volume change after the second re-implantation with breast tumor cancer.

FIG. 204 is pictures of re-implantation of tumor-tissues in nude mice.

FIG. 205 is a picture of a mouse implanted with a re-implanted tumor.

FIG. 206 is a graph of tumor weight for a mice re-implanted with tumors.

FIG. 207 is a picture of a mouse showing no tumor at the dissected at the implant site.

FIG. 208 is a picture of a mouse with no tumor after implantation.

FIG. 209 is pictures of nude mice re-implanted with tumor tissues.

FIG. 210 is a picture of a nude naïve mouse at 8 weeks old used as a negative normal control.

FIG. 211 is a picture of a nude naïve mouse at 8 weeks old used as a negative normal control.

FIG. 212 is a picture of a mouse used in tumor studies.

FIG. 213 is a graph depicting the percentages of B cells in peripheral blood.

FIG. 214 is a graph depicting the percentages of activated B lymphocytes in peripheral blood.

FIG. 215 is a graph depicting the percentages of monocytes and macrophages in peripheral blood.

FIG. 216 is a graph depicting the percentages of mDC and pDC in peripheral blood.

FIG. 217 is a graph depicting the percentages of CD3+ T cells in the spleen.

FIG. 218 is a graph depicting the percentages of B cells in the spleen.

FIG. 219 is a graph depicting the percentages of mDc and pDc in the spleen.

FIG. 220 is a graph of the percentages of activated B lymphocytes in the spleen.

FIG. 221 is a graph of the percentages of monocytes and macrophages in the spleen.

FIG. 222 is a graph of the percentages of granulocytes in the spleen.

FIG. 223 is a graph of percentages of CD3+ T cells in the draining lymph nodes.

FIG. 224 is a graph of the percentages of B cells in the draining lymph nodes.

FIG. 225 is a graph of the percentages of mDC and pDC in the draining lymph nodes.

FIG. 226 is a graph of the percentages of granulocytes in the draining lymph nodes.

FIG. 227 is a graph of the percentages of monocytes and macrophages in the draining lymph nodes.

FIG. 228 is a graph of the percentages of activated B lymphocytes in the draining lymph nodes.

FIG. 229 is a graph of APOE KO mice by area of atherosclerosis of mice treated with various doses of ApoA1.

FIG. 230 is a table showing the inhibition of inflammation factors RNA transcription.

FIG. 231 is a picture of a western blot construct.

FIG. 232 is a graph showing optical density in the APOA1 mouse group.

FIG. 233 is a graph showing optical density in APOA1 vehicle mouse groups.

FIG. 234 is a graph of lipid change in LDLR knock out mice.

FIG. 235 is a graph of lipid change in LDLR knock out mice.

FIG. 236 is a graph depicting the percentage of lesion area for LDLR knock out mice.

FIG. 237-242 are PET/CT scans.

FIG. 243 is a flow chart depicting the process of purifying blood plasma Fraction IV and extracting APOA1 from Fraction IV.

FIG. 244 is a table showing changes in body weight in tumor treated groups with various purified blood plasma products.

### DETAIL DESCRIPTION OF THE INVENTION

55 proteins (defined in this application as KH1-55) have been isolated and identified in a variety of human plasmas. 538 functions, processes, and components for these proteins have been determined. In certain embodiments of the current invention the discovery of these proteins and their unique characteristics has led to the development purified plasma products containing KH proteins and methods of use for treating and preventing a wide range of diseases and infections.

KH1—newly discovered protein synthesized by good healthy KH cells is found in Cryoprecipitate which is used to make HemoRAAS® (Factor VIII) and FibroRAAS® (Fibrinogen) for preventing and/or treating a wide variety of diseases, cancers, infections and other physical conditions and disorders and for maintaining health.

KH2—newly discovered protein synthesized by good healthy KH cells is found in Cryoprecipitate which is used to make HemoRAAS® (Factor VIII) and FibroRAAS® (Fibrinogen) for preventing and/or treating a wide variety of diseases, cancers, infections and other physical conditions and disorders and for maintaining health.

KH3—newly discovered protein synthesized by good healthy KH cells is found in Fraction III which is used to make ProthoRAAS® (Prothrombin complex concentrate), all 16 AFCC RAAS 1® through AFCC RAAS 16® developed products and Immunoglobulin for preventing and/or treating a wide variety of diseases, cancers, infections and other physical conditions and disorders and for maintaining health.

KH4—newly discovered protein synthesized by good healthy KH cells is found in Fraction III which is used to make ProthoRAAS® (Prothrombin complex concentrate), all 16 AFCC RAAS 1® through AFCC RAAS 16® developed products and Immunoglobulin for preventing and/or treating a wide variety of diseases, cancers, infections and other physical conditions and disorders and for maintaining health.

KH5—newly discovered protein synthesized by good healthy KH cells is found in Fraction III which is used to make ProthoRAAS® (Prothrombin complex concentrate), all 16 AFCC RAAS 1® through AFCC RAAS 16® developed products and Immunoglobulin for preventing and/or treating a wide variety of diseases, cancers, infections and other physical conditions and disorders and for maintaining health.

KH6—newly discovered protein synthesized by good healthy KH cells is found in Fraction III which is used to make ProthoRAAS® (Prothrombin complex concentrate), all 16 AFCC RAAS 1® through AFCC RAAS 16® developed products and Immunoglobulin for preventing and/or treating a wide variety of diseases, cancers, infections and other physical conditions and disorders and for maintaining health.

KH7—newly discovered protein synthesized by good healthy KH cells is found in Fraction III which is used to make ProthoRAAS® (Prothrombin complex concentrate), all 16 AFCC RAAS 1® through AFCC RAAS 16® developed products and Immunoglobulin for preventing and/or treating a wide variety of diseases, cancers, infections and other physical conditions and disorders and for maintaining health.

KH8—newly discovered protein synthesized by good healthy KH cells is found in Fraction III which is used to make ProthoRAAS® (Prothrombin complex concentrate), all 16 AFCC RAAS 1® through AFCC RAAS 16® developed products and Immunoglobulin for preventing and/or treating a wide variety of diseases, cancers, infections and other physical conditions and disorders and for maintaining health.

KH9—newly discovered protein synthesized by good healthy KH cells is found in Fraction III which is used to make ProthoRAAS® (Prothrombin complex concentrate), all 16 AFCC RAAS 1® through AFCC RAAS 16® developed products and Immunoglobulin for preventing and/or treating a wide variety of diseases, cancers, infections and other physical conditions and disorders and for maintaining health.

KH10—newly discovered protein synthesized by good healthy KH cells is found in Fraction III which is used to make ProthoRAAS® (Prothrombin complex concentrate), all 16 AFCC RAAS 1® through AFCC RAAS 16® developed products and Immunoglobulin for preventing and/or treating a wide variety of diseases, cancers, infections and other physical conditions and disorders and for maintaining health.

KH11—newly discovered protein synthesized by good healthy KH cells is found in ProthroRAAS® (Prothrombin complex concentrate) for preventing and/or treating a wide variety of diseases, cancers, infections and other physical conditions and disorders and for maintaining health.

KH12—newly discovered protein synthesized by good healthy KH cells is found in ProthroRAAS® (Prothrombin complex concentrate) for preventing and/or treating a wide variety of diseases, cancers, infections and other physical conditions and disorders and for maintaining health.

KH13—newly discovered protein synthesized by good healthy KH cells is found in ProthroRAAS® (Prothrombin complex concentrate) for preventing and/or treating a wide variety of diseases, cancers, infections and other physical conditions and disorders and for maintaining health.

KH14—newly discovered protein synthesized by good healthy KH cells is found in ProthroRAAS® (Prothrombin complex concentrate) for preventing and/or treating a wide variety of diseases, cancers, infections and other physical conditions and disorders and for maintaining health.

KH15—newly discovered protein synthesized by good healthy KH cells is found in ProthroRAAS® (Prothrombin complex concentrate) for preventing and/or treating a wide variety of diseases, cancers, infections and other physical conditions and disorders and for maintaining health.

KH16—newly discovered protein synthesized by good healthy KH cells is found in ProthroRAAS® (Prothrombin complex concentrate) for preventing and/or treating a wide variety of diseases, cancers, infections and other physical conditions and disorders and for maintaining health.

KH17—newly discovered protein synthesized by good healthy KH cells is found in ProthroRAAS® (Prothrombin complex concentrate) for preventing and/or treating a wide variety of diseases, cancers, infections and other physical conditions and disorders and for maintaining health.

KH18—newly discovered protein synthesized by good healthy KH cells is found in ProthroRAAS® (Prothrombin complex concentrate) for preventing and/or treating a wide variety of diseases, cancers, infections and other physical conditions and disorders and for maintaining health.

KH19—newly discovered protein synthesized by good healthy KH cells is found in AFCC KH® for preventing and/or treating a wide variety of diseases, cancers, infections and other physical conditions and disorders and for maintaining health.

KH20—newly discovered protein synthesized by good healthy KH cells is found in AFCC KH® for preventing and/or treating a wide variety of diseases, cancers, infections and other physical conditions and disorders and for maintaining health.

KH21—newly discovered protein synthesized by good healthy KH cells is found in fraction IV which is used to manufacture human Albumin, APOA1, Transferrin, Alpha1 Antitripsin, Anti Thrombin III, CP98 and 16 AFOD RAAS 1® through AFOD RAAS 16® for preventing and/or treating a wide variety of diseases, cancers, infections and other physical conditions and disorders and for maintaining health.

KH22—newly discovered protein synthesized by good healthy KH cells is found in fraction IV which is used to manufacture human Albumin, APOA1, Transferrin, Alpha1 Antitripsin, Anti Thrombin III, CP98 and 16 AFOD RAAS 1® through AFOD RAAS 16® for preventing and/or treating a wide variety of diseases, cancers, infections and other physical conditions and disorders and for maintaining health.

KH23—newly discovered protein synthesized by good healthy KH cells is found in fraction IV which is used to manufacture human Albumin, APOA1, Transferrin, Alpha1 Antitripsin, Anti Thrombin III, CP98 and 16 AFOD RAAS 1® through AFOD RAAS 16® for preventing and/or treating a wide variety of diseases, cancers, infections and other physical conditions and disorders and for maintaining health.

KH24—newly discovered protein synthesized by good healthy KH cells is found in AFOD KH® for preventing and/or treating a wide variety of diseases, cancers, infections and other physical conditions and disorders and for maintaining health.

KH25—newly discovered protein synthesized by good healthy KH cells is found in AFOD KH® for preventing and/or treating a wide variety of diseases, cancers, infections and other physical conditions and disorders and for maintaining health.

KH26—newly discovered protein synthesized by good healthy KH cells is found in AFOD KH® for preventing and/or treating a wide variety of diseases, cancers, infections and other physical conditions and disorders and for maintaining health.

KH27—newly discovered protein synthesized by good healthy KH cells is found in AFOD KH® for preventing and/or treating a wide variety of diseases, cancers, infections and other physical conditions and disorders and for maintaining health.

KH28—newly discovered protein synthesized by good healthy KH cells is found in HemoRAAS® (Human Factor VIII) for preventing and/or treating a wide variety of diseases, cancers, infections and other physical conditions and disorders and for maintaining health.

KH29—newly discovered protein synthesized by good healthy KH cells is found in HemoRAAS® (Human Factor VIII) for preventing and/or treating a wide variety of diseases, cancers, infections and other physical conditions and disorders and for maintaining health.

KH30—newly discovered protein synthesized by good healthy KH cells is found in FibroRAAS® (Human Fibrinogen) for preventing and/or treating a wide variety of diseases, cancers, infections and other physical conditions and disorders and for maintaining health.

KH31—newly discovered protein synthesized by good healthy KH cells is found in FibroRAAS® (Human Fibrinogen) for preventing and/or treating a wide variety of diseases, cancers, infections and other physical conditions and disorders and for maintaining health.

KH32—newly discovered protein synthesized by good healthy KH cells is found in FibroRAAS® (Human Fibrinogen) for preventing and/or treating a wide variety of diseases, cancers, infections and other physical conditions and disorders and for maintaining health.

KH33—newly discovered protein synthesized by good healthy KH cells is found in GammaRAAS® (Human Immunoglobulin) for preventing and/or treating a wide variety of diseases, cancers, infections and other physical conditions and disorders and for maintaining health.

KH34—newly discovered protein synthesized by good healthy KH cells is found in GammaRAAS® (Human Immunoglobulin) for preventing and/or treating a wide variety of diseases, cancers, infections and other physical conditions and disorders and for maintaining health.

KH35—newly discovered protein synthesized by good healthy KH cells is found in GammaRAAS® (Human Immunoglobulin) preventing and/or for treating a wide variety of diseases, cancers, infections and other physical conditions and disorders and for maintaining health.

KH36—newly discovered protein synthesized by good healthy KH cells is found in GammaRAAS® (Human Immunoglobulin) for preventing and/or, treating a wide variety of diseases, cancers, infections and other physical conditions and disorders and for maintaining health.

KH37—newly discovered protein synthesized by good healthy KH cells is found in GammaRAAS® (Human Immunoglobulin) for preventing and/or treating a wide variety of diseases, cancers, infections and other physical conditions and disorders and for maintaining health.

KH38—newly discovered protein synthesized by good healthy KH cells is found in AFCC RAAS 3® through 16 for preventing and/or treating a wide variety of diseases, cancers, infections and other physical conditions and disorders and for maintaining health.

KH39—newly discovered protein synthesized by good healthy KH cells is found in AFCC RAAS 3® through 16 for preventing and/or treating a wide variety of diseases, cancers, infections and other physical conditions and disorders and for maintaining health.

KH40—newly discovered protein synthesized by good healthy KH cells is found in AFCC RAAS 3® through 16 for preventing and/or treating a wide variety of diseases, cancers, infections and other physical conditions and disorders and for maintaining health.

KH41—newly discovered protein synthesized by good healthy KH cells is found in Fraction III which is used to make ProthoRAAS® (Prothrombin complex concentrate), all 16 AFCC RAAS 10 through AFCC RAAS 16® developed products and Immunoglobulin for preventing and/or treating a wide variety of diseases, cancers, infections and other physical conditions and disorders and for maintaining health.

KH42—newly discovered protein synthesized by good healthy KH cells is found in Fraction III which is used to make ProthoRAAS® (Prothrombin complex concentrate), all 16 AFCC RAAS 10 through AFCC RAAS 16® developed products and Immunoglobulin for preventing and/or treating a wide variety of diseases, cancers, infections and other physical conditions and disorders and for maintaining health.

KH43—newly discovered protein synthesized by good healthy KH cells is found in Fraction III which is used to make ProthoRAAS® (Prothrombin complex concentrate), all 16 AFCC RAAS 10 through AFCC RAAS 16® developed products and Immunoglobulin for preventing and/or treating a wide variety of diseases, cancers, infections and other physical conditions and disorders and for maintaining health.

KH44—newly discovered protein synthesized by good healthy KH cells is found in ThrombiRAAS® (Human Thrombin) for preventing and/or treating a wide variety of diseases, cancers, infections and other physical conditions and disorders and for maintaining health.

KH45—newly discovered protein synthesized by good healthy KH cells is found in ThrombiRAAS® (Human Thrombin) for preventing and/or treating a wide variety of diseases, cancers, infections and other physical conditions and disorders and for maintaining health.

KH46—newly discovered protein synthesized by good healthy KH cells is found in ThrombiRAAS® (Human Thrombin) for preventing and/or treating a wide variety of diseases, cancers, infections and other physical conditions and disorders and for maintaining health.

KH47—newly discovered protein synthesized by good healthy KH cells is found in ThrombiRAAS® (Human Thrombin) for preventing and/or treating a wide variety of diseases, cancers, infections and other physical conditions and disorders and for maintaining health.

KH48—newly discovered protein synthesized by good healthy KH cells is found in AFOD RAAS 1® through AFOD RAAS 16® for preventing and/or treating a wide variety of diseases, cancers, infections and other physical conditions and disorders and for maintaining health.

KH49—newly discovered protein synthesized by good healthy KH cells is found in AFOD RAAS 10 through AFOD RAAS 16® for preventing and/or treating a wide variety of diseases, cancers infections and other physical conditions and disorders and for maintaining health.

KH50—newly discovered protein synthesized by good healthy KH cells is found in AFOD RAAS 1® through AFOD RAAS 16® for preventing and/or treating a wide variety of diseases, cancers, infections and other physical conditions and disorders and for maintaining health.

KH51—newly discovered protein synthesized by good healthy KH cells is found in AlbuRAAS® (Human Albumin) for preventing and/or treating a wide variety of diseases, cancers, infections and other physical conditions and disorders and for maintaining health.

KH52—newly discovered protein synthesized by good healthy KH cells is found in FibringluRAAS® (Human high concentrate Fibrinogen) for preventing and/or treating a wide variety of diseases, cancers, infections and other physical conditions and disorders and for maintaining health.

KH53—newly discovered protein synthesized by good healthy KH cells is found in AFCC RAAS 2® (Fraction IV) for preventing and/or treating a wide variety of diseases, cancers, infections and other physical conditions and disorders and for maintaining health.

KH54—newly discovered protein synthesized by good healthy KH cells is found in Transferrin for preventing and/or treating a wide variety of diseases, cancers, infections and other physical conditions and disorders and for maintaining health.

KH55—newly discovered protein synthesized by good healthy KH cells is found in Transferrin for preventing and/or treating a wide variety of diseases, cancers, infections and other physical conditions and disorders and for maintaining health.

Embodiments of the invention include KH proteins found in purified blood plasma products including:

AFOD comprising CP 98 kDa protein, CP reuloplasmin, KRT2 Keratin, type II cytoskeletal epidermal, KH22, KH23, KH24, KH25, APOA1 Apolipoprotein A1, human albumin, transferrin, vimentin, and haptoglobin;

AFCC comprising C3 complement C3, ENO1 Isoform ENO1, TUFM elongation factor, ASS1 argininosuccinate, ANXA2 isoform 2 of annexin A2, glyceraldehyde-3-phosphate dehydrogenase, KHT 86 keratin, type II cuticular HB6, KH20, LDHA isoform 1 of L-lactate dehydrogenase A chain, fibrin beta, KH21, growth inhibiting protein 25, fibrinogen gamma, chain L crystal structure of human fibrinogen, chain A of IgM, chain A crystal structure of the Fab fragment of a human monoclonal Igm cold agglutinin, immunoglobulin light chain, and chain C molecular basis for complement recognition;

AFOD KH comprising CP 98 kDa, CP ceruloplasmin, KRT2 keratin type II cytoskeletal 2 epidermal, KH proteins, APOA1, human albumin, transferrin, vimentin, and haptoglobin;

AFOD RAAS 8 (also known as AFOD RAAS 104) comprising TF serotransferrin derived from fraction III WIG;

AFOD RAAS 101 comprising ALB uncharacterized protein, HPR 31 kDa protein, albumin uncharacterized protein, AIBG isoform 1 of alpha-1B-glycoprotein, HPR haptoglobin, and KH51;

AFOD RAAS 102 (main component of immunoglobulin) comprising 120/E19 IGHV4-31, IGHG1 44 kDa, 191/H18 IGHV4 31, IGHG1 32 kDa, IGHG1 putative uncharacterized protein, DKFZp686G11190, and KH proteins 33-37;

AFOD RAAS 107 comprising protein 1CP 98 kDa including NUP98 and Nup 96, which play a role in bidirectional transport;

AFOD RAAS 109 comprising transferrin and KH proteins 21-27 and KH proteins 48-50; AFOD RAAS 110 comprising anti-thrombin III and KH proteins 22-27 and KH proteins 48-50; and

AFCC RAAS 1 (also known as AFCC RAAS 105) comprising factor II, factor VII, factor IX, factor X, and KH proteins 111-118.

In certain embodiments any two or many of these new found proteins KH1 through KH55 synthesized by good healthy KH cells are combined for preventing and/or treating a wide variety of diseases, cancers, infections and other physical conditions and disorders and for maintaining health.

Embodiments of the invention include any recombinant DNA or many of these new found proteins KH1 through KH55 synthesized by good healthy KH cells are combined for preventing and/or treating a wide variety of diseases, cancers, infections and other physical conditions and disorders and for maintaining health.

In other embodiments any monoclonal or many of these new found proteins KH1 through KH55 synthesized by good healthy KH cells are combined for preventing and/or treating a wide variety of diseases, cancers, infections and other physical conditions and disorders and for maintaining health.

Another embodiment of the invention include processes for the isolating, purifying and concentrating of any KH1 to KH55 protein, and/or the combination of more than of KH1 to KH 55 from all natural products, recombinant DNA, cDNA, or synthesized products for preventing and/or treating a wide variety of diseases, cancers, infections and other physical conditions and disorders and for maintaining health.

Certain embodiments also include the use of any of these KH proteins in combination, either singly or more than two with any natural products, products from recombinant DNA, engineered DNA, cDNA and for preventing and/or treating a wide variety of diseases, cancers, infections and other physical conditions and disorders and for maintaining health.

Other embodiments encompass the use of any of these KH proteins in combination, either singly or more than two KH proteins with any chemical products, medication, small molecules, any future medication for preventing and/or treating a wide variety of diseases, cancers, infections and other physical conditions and disorders and for maintaining health.

Another embodiment of the invention includes the process to isolating and producing and use a mixture of any of these KH proteins in combination, either singly or more than two KH proteins with any natural products or synthesized products as part of nutrition, food, and/or supplemental products in any capacity for preventing and/or treating a wide variety of diseases, cancers, infections and other physical conditions and disorders and for maintaining health.

538 functions have been identified for the 55 KH proteins, which provide them with unique characteristics for treating a wide range of disease, infection, and other cellular disturbances as expressed in some embodiments of the invention as described.

| | | Fraction - P | | |
|---|---|---|---|---|
| | | (process), C | | |
| | | (component), F | Sequence | |
| Number | GI code | (function) | name | Sequence desc. |
| KH 1 | 21749960 | cryopaste | gi\|21749960 | dock4_humandedicator of |
| | | | | cytokinesis protein 4 |
| | | | | os = homo sapiens gn = dock4 |
| | | | | pe = 1 sv = 3 |
| gi\|21749960 | dock4_humandedicator of | F | GO: 0005102 | receptor binding |
| | cytokinesis protein 4 | | | |
| | os = homo sapiens gn = dock4 | | | |
| | pe = 1 sv = 3 | | | |
| gi\|21749960 | dock4_humandedicator of | P | GO: 0043547 | positive regulation of |
| | cytokinesis protein 4 | | | GTPase activity |
| | os = homo sapiens gn = dock4 | | | |
| | pe = 1 sv = 3 | | | |
| gi\|21749960 | dock4_humandedicator of | P | GO: 0016477 | cell migration |
| | cytokinesis protein 4 | | | |
| | os = homo sapiens gn = dock4 | | | |
| | pe = 1 sv = 3 | | | |
| gi\|21749960 | dock4_humandedicator of | P | GO: 0007165 | signal transduction |
| | cytokinesis protein 4 | | | |
| | os = homo sapiens gn = dock4 | | | |
| | pe = 1 sv = 3 | | | |
| gi\|21749960 | dock4_humandedicator of | P | GO: 0006935 | chemotaxis |
| | cytokinesis protein 4 | | | |
| | os = homo sapiens gn = dock4 | | | |
| | pe = 1 sv = 3 | | | |
| gi\|21749960 | dock4_humandedicator of | C | GO: 0005737 | cytoplasm |
| | cytokinesis protein 4 | | | |
| | os = homo sapiens gn = dock4 | | | |
| | pe = 1 sv = 3 | | | |
| gi\|21749960 | dock4_humandedicator of | F | GO: 0005083 | small GTPase regulator |
| | cytokinesis protein 4 | | | activity |
| | os = homo sapiens gn = dock4 | | | |
| | pe = 1 sv = 3 | | | |
| gi\|21749960 | dock4_humandedicator of | F | GO: 0019904 | protein domain specific |
| | cytokinesis protein 4 | | | binding |
| | os = homo sapiens gn = dock4 | | | |
| | pe = 1 sv = 3 | | | |
| gi\|21749960 | dock4_humandedicator of | P | GO: 0048583 | regulation of response to |
| | cytokinesis protein 4 | | | stimulus |
| | os = homo sapiens gn = dock4 | | | |
| | pe = 1 sv = 3 | | | |
| gi\|21749960 | dock4_humandedicator of | F | GO: 0005096 | GTPase activator activity |
| | cytokinesis protein 4 | | | |
| | os = homo sapiens gn = dock4 | | | |
| | pe = 1 sv = 3 | | | |
| gi\|21749960 | dock4_humandedicator of | F | GO: 0051020 | GTPase binding |
| | cytokinesis protein 4 | | | |
| | os = homo sapiens gn = dock4 | | | |
| | pe = 1 sv = 3 | | | |
| gi\|21749960 | dock4_humandedicator of | C | GO: 0016020 | membrane |
| | cytokinesis protein 4 | | | |
| | os = homo sapiens gn = dock4 | | | |
| | pe = 1 sv = 3 | | | |
| KH 2 | 215415640 | cryopaste | gi\|215415640 | apoa1_humanapolipoprotein |
| | | | | a-i os = homo sapiens |
| | | | | gn = apoa1 pe = 1 sv = 1 |
| gi\|215415640 | apoa1_humanapolipoprotein | P | GO: 0070508 | cholesterol import |
| | a-i os = homo sapiens | | | |
| | gn = apoa1 pe = 1 sv = 1 | | | |
| gi\|215415640 | apoa1_humanapolipoprotein | C | GO: 0030139 | endocytic vesicle |
| | a-i os = homo sapiens | | | |
| | gn = apoa1 pe = 1 sv = 1 | | | |
| gi\|215415640 | apoa1_humanapolipoprotein | P | GO: 0050728 | negative regulation of |
| | a-i os = homo sapiens | | | inflammatory response |
| | gn = apoa1 pe = 1 sv = 1 | | | |
| gi\|215415640 | apoa1_humanapolipoprotein | P | GO: 0033344 | cholesterol efflux |
| | a-i os = homo sapiens | | | |
| | gn = apoa1 pe = 1 sv = 1 | | | |
| gi\|215415640 | apoa1_humanapolipoprotein | P | GO: 0034115 | negative regulation of |
| | a-i os = homo sapiens | | | heterotypic cell-cell |
| | gn = apoa1 pe = 1 sv = 1 | | | adhesion |
| gi\|215415640 | apoa1_humanapolipoprotein | P | GO: 0018206 | peptidyl-methionine |
| | a-i os = homo sapiens | | | modification |
| | gn = apoa1 pe = 1 sv = 1 | | | |
| gi\|215415640 | apoa1_humanapolipoprotein | P | GO: 0042157 | lipoprotein metabolic |
| | a-i os = homo sapiens | | | process |
| | gn = apoa1 pe = 1 sv = 1 | | | |
| gi\|215415640 | apoa1_humanapolipoprotein | P | GO: 0043691 | reverse cholesterol transport |
| | a-i os = homo sapiens | | | |
| | gn = apoa1 pe = 1 sv = 1 | | | |
| gi\|215415640 | apoa1_humanapolipoprotein | F | GO: 0005543 | phospholipid binding |
| | a-i os = homo sapiens | | | |
| | gn = apoa1 pe = 1 sv = 1 | | | |
| gi\|215415640 | apoa1_humanapolipoprotein | P | GO: 0002740 | negative regulation of |
| | a-i os = homo sapiens | | | cytokine secretion involved |
| | gn = apoa1 pe = 1 sv = 1 | | | in immune response |
| gi\|215415640 | apoa1_humanapolipoprotein | P | GO: 0008203 | cholesterol metabolic |
| | a-i os = homo sapiens | | | process |
| | gn = apoa1 pe = 1 sv = 1 | | | |
| gi\|215415640 | apoa1_humanapolipoprotein | P | GO: 0050713 | negative regulation of |
| | a-i os = homo sapiens | | | interleukin-1 beta secretion |
| | gn = apoa1 pe = 1 sv = 1 | | | |
| gi\|215415640 | apoa1_humanapolipoprotein | P | GO: 0018158 | protein oxidation |
| | a-i os = homo sapiens | | | |
| | gn = apoa1 pe = 1 sv = 1 | | | |
| gi\|215415640 | apoa1_humanapolipoprotein | P | GO: 0006656 | phosphatidylcholine |
| | a-i os = homo sapiens | | | biosynthetic process |
| | gn = apoa1 pe = 1 sv = 1 | | | |
| gi\|215415640 | apoa1_humanapolipoprotein | F | GO: 0001540 | beta-amyloid binding |
| | a-i os = homo sapiens | | | |
| | gn = apoa1 pe = 1 sv = 1 | | | |
| gi\|215415640 | apoa1_humanapolipoprotein | F | GO: 0060228 | phosphatidylcholine-sterol |
| | a-i os = homo sapiens | | | O-acyltransferase activator |
| | gn = apoa1 pe = 1 sv = 1 | | | activity |
| gi\|215415640 | apoa1_humanapolipoprotein | P | GO: 0042632 | cholesterol homeostasis |
| | a-i os = homo sapiens | | | |
| | gn = apoa1 pe = 1 sv = 1 | | | |
| gi\|215415640 | apoa1_humanapolipoprotein | F | GO: 0015485 | cholesterol binding |
| | a-i os = homo sapiens | | | |
| | gn = apoa1 pe = 1 sv = 1 | | | |
| gi\|215415640 | apoa1_humanapolipoprotein | P | GO: 0042060 | wound healing |
| | a-i os = homo sapiens | | | |
| | gn = apoa1 pe = 1 sv = 1 | | | |
| gi\|215415640 | apoa1_humanapolipoprotein | F | GO: 0034191 | apolipoprotein A-I receptor |
| | a-i os = homo sapiens | | | binding |
| | gn = apoa1 pe = 1 sv = 1 | | | |
| gi\|215415640 | apoa1_humanapolipoprotein | F | GO: 0042802 | identical protein binding |
| | a-i os = homo sapiens | | | |
| | gn = apoa1 pe = 1 sv = 1 | | | |
| gi\|215415640 | apoa1_humanapolipoprotein | P | GO: 0010903 | negative regulation of very- |
| | a-i os = homo sapiens | | | low-density lipoprotein |
| | gn = apoa1 pe = 1 sv = 1 | | | particle remodeling |
| gi\|215415640 | apoa1_humanapolipoprotein | P | GO: 0010804 | negative regulation of tumor |
| | a-i os = homo sapiens | | | necrosis factor-mediated |
| | gn = apoa1 pe = 1 sv = 1 | | | signaling pathway |
| gi\|215415640 | apoa1_humanapolipoprotein | P | GO: 0034380 | high-density lipoprotein |
| | a-i os = homo sapiens | | | particle assembly |
| | gn = apoa1 pe = 1 sv = 1 | | | |
| gi\|215415640 | apoa1_humanapolipoprotein | P | GO: 0007186 | G-protein coupled receptor |
| | a-i os = homo sapiens | | | signaling pathway |
| | gn = apoa1 pe = 1 sv = 1 | | | |
| gi\|215415640 | apoa1_humanapolipoprotein | P | GO: 0050821 | protein stabilization |
| | a-i os = homo sapiens | | | |
| | gn = apoa1 pe = 1 sv = 1 | | | |
| gi\|215415640 | apoa1_humanapolipoprotein | C | GO: 0034361 | very-low-density lipoprotein |
| | a-i os = homo sapiens | | | particle |
| | gn = apoa1 pe = 1 sv = 1 | | | |
| gi\|215415640 | apoa1_humanapolipoprotein | P | GO: 0032488 | Cdc42 protein signal |
| | a-i os = homo sapiens | | | transduction |
| | gn = apoa1 pe = 1 sv = 1 | | | |
| gi\|215415640 | apoa1_humanapolipoprotein | P | GO: 0060354 | negative regulation of cell |
| | a-i os = homo sapiens | | | adhesion molecule |
| | gn = apoa1 pe = 1 sv = 1 | | | production |
| gi\|215415640 | apoa1_humanapolipoprotein | P | GO: 0055091 | phospholipid homeostasis |
| | a-i os = homo sapiens | | | |
| | gn = apoa1 pe = 1 sv = 1 | | | |
| gi\|215415640 | apoa1_humanapolipoprotein | P | GO: 0010873 | positive regulation of |
| | a-i os = homo sapiens | | | cholesterol esterification |
| | gn = apoa1 pe = 1 sv = 1 | | | |
| gi\|215415640 | apoa1_humanapolipoprotein | F | GO: 0017127 | cholesterol transporter |
| | a-i os = homo sapiens | | | activity |
| | gn = apoa1 pe = 1 sv = 1 | | | |
| gi\|215415640 | apoa1_humanapolipoprotein | F | GO: 0019899 | enzyme binding |
| | a-i os = homo sapiens | | | |
| | gn = apoa1 pe = 1 sv = 1 | | | |
| gi\|215415640 | apoa1_humanapolipoprotein | F | GO: 0070653 | high-density lipoprotein |
| | a-i os = homo sapiens | | | particle receptor binding |
| | gn = apoa1 pe = 1 sv = 1 | | | |
| gi\|215415640 | apoa1_humanapolipoprotein | P | GO: 0070328 | triglyceride homeostasis |
| | a-i os = homo sapiens | | | |
| | gn = apoa1 pe = 1 sv = 1 | | | |
| gi\|215415640 | apoa1_humanapolipoprotein | C | GO: 0034366 | spherical high-density |
| | a-i os = homo sapiens | | | lipoprotein particle |
| | gn = apoa1 pe = 1 sv = 1 | | | |
| gi\|215415640 | apoa1_humanapolipoprotein | P | GO: 0033700 | phospholipid efflux |
| | a-i os = homo sapiens | | | |
| | gn = apoa1 pe = 1 sv = 1 | | | |
| gi\|215415640 | apoa1_humanapolipoprotein | P | GO: 0051345 | positive regulation of |
| | a-i os = homo sapiens | | | hydrolase activity |
| | gn = apoa1 pe = 1 sv = 1 | | | |
| KH 3 | 215415638 | Fr III | gi\|215415638 | apoa1_humanapolipoprotein |
| | | | | a-i os = homo sapiens |
| | | | | gn = apoa1 pe = 1 sv = 1 |
| gi\|215415638 | apoa1_humanapolipoprotein | P | GO: 0070508 | cholesterol import |
| | a-i os = homo sapiens | | | |
| | gn = apoa1 pe = 1 sv = 1 | | | |
| gi\|215415638 | apoa1_humanapolipoprotein | C | GO: 0030139 | endocytic vesicle |
| | a-i os = homo sapiens | | | |
| | gn = apoa1 pe = 1 sv = 1 | | | |
| gi\|215415638 | apoa1_humanapolipoprotein | P | GO: 0050728 | negative regulation of |
| | a-i os = homo sapiens | | | inflammatory response |
| | gn = apoa1 pe = 1 sv = 1 | | | |
| gi\|215415638 | apoa1_humanapolipoprotein | P | GO: 0033344 | cholesterol efflux |
| | a-i os = homo sapiens | | | |
| | gn = apoa1 pe = 1 sv = 1 | | | |
| gi\|215415638 | apoa1_humanapolipoprotein | P | GO: 0034115 | negative regulation of |
| | a-i os = homo sapiens | | | heterotypic cell-cell |
| | gn = apoa1 pe = 1 sv = 1 | | | adhesion |
| gi\|215415638 | apoa1_humanapolipoprotein | P | GO: 0018206 | peptidyl-methionine |
| | a-i os = homo sapiens | | | modification |
| | gn = apoa1 pe = 1 sv = 1 | | | |
| gi\|215415638 | apoa1_humanapolipoprotein | P | GO: 0042157 | lipoprotein metabolic |
| | a-i os = homo sapiens | | | process |
| | gn = apoa1 pe = 1 sv = 1 | | | |
| gi\|215415638 | apoa1_humanapolipoprotein | P | GO: 0043691 | reverse cholesterol transport |
| | a-i os = homo sapiens | | | |
| | gn = apoa1 pe = 1 sv = 1 | | | |
| gi\|215415638 | apoa1_humanapolipoprotein | F | GO: 0005543 | phospholipid binding |
| | a-i os = homo sapiens | | | |
| | gn = apoa1 pe = 1 sv = 1 | | | |
| gi\|215415638 | apoa1_humanapolipoprotein | P | GO: 0002740 | negative regulation of |
| | a-i os = homo sapiens | | | cytokine secretion involved |
| | gn = apoa1 pe = 1 sv = 1 | | | in immune response |
| gi\|215415638 | apoa1_humanapolipoprotein | P | GO: 0008203 | cholesterol metabolic |
| | a-i os = homo sapiens | | | process |
| | gn = apoa1 pe = 1 sv = 1 | | | |
| gi\|215415638 | apoa1_humanapolipoprotein | P | GO: 0050713 | negative regulation of |
| | a-i os = homo sapiens | | | interleukin-1 beta secretion |
| | gn = apoa1 pe = 1 sv = 1 | | | |
| gi\|215415638 | apoa1_humanapolipoprotein | P | GO: 0018158 | protein oxidation |
| | a-i os = homo sapiens | | | |
| | gn = apoa1 pe = 1 sv = 1 | | | |
| gi\|215415638 | apoa1_humanapolipoprotein | P | GO: 0006656 | phosphatidylcholine |
| | a-i os = homo sapiens | | | biosynthetic process |
| | gn = apoa1 pe = 1 sv = 1 | | | |
| gi\|215415638 | apoa1_humanapolipoprotein | F | GO: 0001540 | beta-amyloid binding |
| | a-i os = homo sapiens | | | |
| | gn = apoa1 pe = 1 sv = 1 | | | |
| gi\|215415638 | apoa1_humanapolipoprotein | F | GO: 0060228 | phosphatidylcholine-sterol |
| | a-i os = homo sapiens | | | O-acyltransferase activator |
| | gn = apoa1 pe = 1 sv = 1 | | | activity |
| gi\|215415638 | apoa1_humanapolipoprotein | P | GO: 0042632 | cholesterol homeostasis |
| | a-i os = homo sapiens | | | |
| | gn = apoa1 pe = 1 sv = 1 | | | |
| gi\|215415638 | apoa1_humanapolipoprotein | F | GO: 0015485 | cholesterol binding |
| | a-i os = homo sapiens | | | |
| | gn = apoa1 pe = 1 sv = 1 | | | |
| gi\|215415638 | apoa1_humanapolipoprotein | P | GO: 0042060 | wound healing |
| | a-i os = homo sapiens | | | |
| | gn = apoa1 pe = 1 sv = 1 | | | |
| gi\|215415638 | apoa1_humanapolipoprotein | F | GO: 0034191 | apolipoprotein A-I receptor |
| | a-i os = homo sapiens | | | binding |
| | gn = apoa1 pe = 1 sv = 1 | | | |
| gi\|215415638 | apoa1_humanapolipoprotein | F | GO: 0042802 | identical protein binding |
| | a-i os = homo sapiens | | | |
| | gn = apoa1 pe = 1 sv = 1 | | | |
| gi\|215415638 | apoa1_humanapolipoprotein | P | GO: 0010903 | negative regulation of very- |
| | a-i os = homo sapiens | | | low-density lipoprotein |
| | gn = apoa1 pe = 1 sv = 1 | | | particle remodeling |
| gi\|215415638 | apoa1_humanapolipoprotein | P | GO: 0010804 | negative regulation of tumor |
| | a-i os = homo sapiens | | | necrosis factor-mediated |
| | gn = apoa1 pe = 1 sv = 1 | | | signaling pathway |
| gi\|215415638 | apoa1_humanapolipoprotein | P | GO: 0034380 | high-density lipoprotein |
| | a-i os = homo sapiens | | | particle assembly |
| | gn = apoa1 pe = 1 sv = 1 | | | |
| gi\|215415638 | apoa1_humanapolipoprotein | P | GO: 0007186 | G-protein coupled receptor |
| | a-i os = homo sapiens | | | signaling pathway |
| | gn = apoa1 pe = 1 sv = 1 | | | |
| gi\|215415638 | apoa1_humanapolipoprotein | P | GO: 0050821 | protein stabilization |
| | a-i os = homo sapiens | | | |
| | gn = apoa1 pe = 1 sv = 1 | | | |
| gi\|215415638 | apoa1_humanapolipoprotein | C | GO: 0034361 | very-low-density lipoprotein |
| | a-i os = homo sapiens | | | particle |
| | gn = apoa1 pe = 1 sv = 1 | | | |
| gi\|215415638 | apoa1_humanapolipoprotein | P | GO: 0032488 | Cdc42 protein signal |
| | a-i os = homo sapiens | | | transduction |
| | gn = apoa1 pe = 1 sv = 1 | | | |
| gi\|215415638 | apoa1_humanapolipoprotein | P | GO: 0060354 | negative regulation of cell |
| | a-i os = homo sapiens | | | adhesion molecule |
| | gn = apoa1 pe = 1 sv = 1 | | | production |
| gi\|215415638 | apoa1_humanapolipoprotein | P | GO: 0055091 | phospholipid homeostasis |
| | a-i os = homo sapiens | | | |
| | gn = apoa1 pe = 1 sv = 1 | | | |
| gi\|215415638 | apoa1_humanapolipoprotein | P | GO: 0010873 | positive regulation of |
| | a-i os = homo sapiens | | | cholesterol esterification |
| | gn = apoa1 pe = 1 sv = 1 | | | |
| gi\|215415638 | apoa1_humanapolipoprotein | F | GO: 0017127 | cholesterol transporter |
| | a-i os = homo sapiens | | | activity |
| | gn = apoa1 pe = 1 sv = 1 | | | |
| gi\|215415638 | apoa1_humanapolipoprotein | F | GO: 0019899 | enzyme binding |
| | a-i os = homo sapiens | | | |
| | gn = apoa1 pe = 1 sv = 1 | | | |
| gi\|215415638 | apoa1_humanapolipoprotein | F | GO: 0070653 | high-density lipoprotein |
| | a-i os = homo sapiens | | | particle receptor binding |
| | gn = apoa1 pe = 1 sv = 1 | | | |
| gi\|215415638 | apoa1_humanapolipoprotein | P | GO: 0070328 | triglyceride homeostasis |
| | a-i os = homo sapiens | | | |
| | gn = apoa1 pe = 1 sv = 1 | | | |
| gi\|215415638 | apoa1_humanapolipoprotein | C | GO: 0034366 | spherical high-density |
| | a-i os = homo sapiens | | | lipoprotein particle |
| | gn = apoa1 pe = 1 sv = 1 | | | |
| gi\|215415638 | apoa1_humanapolipoprotein | P | GO: 0033700 | phospholipid efflux |
| | a-i os = homo sapiens | | | |
| | gn = apoa1 pe = 1 sv = 1 | | | |
| gi\|215415638 | apoa1_humanapolipoprotein | P | GO: 0051345 | positive regulation of |
| | a-i os = homo sapiens | | | hydrolase activity |
| | gn = apoa1 pe = 1 sv = 1 | | | |
| KH 4 | 40044478 | Fr III | | |
| KH 5 | 194383496 | Fr III | gi\|194383496 | thrb_humanprothrombin |
| | | | | os = homo sapiens gn = f2 |
| | | | | pe = 1 sv = 2 |
| gi\|194383496 | thrb_humanprothrombin | P | GO: 0032879 | regulation of localization |
| | os = homo sapiens gn = f2 | | | |
| | pe = 1 sv = 2 | | | |
| gi\|194383496 | thrb_humanprothrombin | P | GO: 0048468 | cell development |
| | os = homo sapiens gn = f2 | | | |
| | pe = 1 sv = 2 | | | |
| gi\|194383496 | thrb_humanprothrombin | P | GO: 2000026 | regulation of multicellular |
| | os = homo sapiens gn = f2 | | | organismal development |
| | pe = 1 sv = 2 | | | |
| gi\|194383496 | thrb_humanprothrombin | P | GO: 0010557 | positive regulation of |
| | os = homo sapiens gn = f2 | | | macromolecule biosynthetic |
| | pe = 1 sv = 2 | | | process |
| gi\|194383496 | thrb_humanprothrombin | P | GO: 0030194 | positive regulation of blood |
| | os = homo sapiens gn = f2 | | | coagulation |
| | pe = 1 sv = 2 | | | |
| gi\|194383496 | thrb_humanprothrombin | F | GO: 0005102 | receptor binding |
| | os = homo sapiens gn = f2 | | | |
| | pe = 1 sv = 2 | | | |
| gi\|194383496 | thrb_humanprothrombin | P | GO: 0009967 | positive regulation of signal |
| | os = homo sapiens gn = f2 | | | transduction |
| | pe = 1 sv = 2 | | | |
| gi\|194383496 | thrb_humanprothrombin | C | GO: 0005615 | extracellular space |
| | os = homo sapiens gn = f2 | | | |
| | pe = 1 sv = 2 | | | |
| gi\|194383496 | thrb_humanprothrombin | P | GO: 0030168 | platelet activation |
| | os = homo sapiens gn = f2 | | | |
| | pe = 1 sv = 2 | | | |
| gi\|194383496 | thrb_humanprothrombin | F | GO: 0008236 | serine-type peptidase activity |
| | os = homo sapiens gn = f2 | | | |
| | pe = 1 sv = 2 | | | |
| gi\|194383496 | thrb_humanprothrombin | P | GO: 0016477 | cell migration |
| | os = homo sapiens gn = f2 | | | |
| | pe = 1 sv = 2 | | | |
| gi\|194383496 | thrb_humanprothrombin | P | GO: 0006508 | proteolysis |
| | os = homo sapiens gn = f2 | | | |
| | pe = 1 sv = 2 | | | |
| gi\|194383496 | thrb_humanprothrombin | P | GO: 0001934 | positive regulation of protein |
| | os = homo sapiens gn = f2 | | | phosphorylation |
| | pe = 1 sv = 2 | | | |
| gi\|194383496 | thrb_humanprothrombin | P | GO: 0007166 | cell surface receptor |
| | os = homo sapiens gn = f2 | | | signaling pathway |
| | pe = 1 sv = 2 | | | |
| gi\|194383496 | thrb_humanprothrombin | P | GO: 0048523 | negative regulation of |
| | os = homo sapiens gn = f2 | | | cellular process |
| | pe = 1 sv = 2 | | | |
| gi\|194383496 | thrb_humanprothrombin | P | GO: 0006810 | transport |
| | os = homo sapiens gn = f2 | | | |
| | pe = 1 sv = 2 | | | |
| gi\|194383496 | thrb_humanprothrombin | P | GO: 0042730 | fibrinolysis |
| | os = homo sapiens gn = f2 | | | |
| | pe = 1 sv = 2 | | | |
| gi\|194383496 | thrb_humanprothrombin | C | GO: 0005622 | intracellular |
| | os = homo sapiens gn = f2 | | | |
| | pe = 1 sv = 2 | | | |
| gi\|194383496 | thrb_humanprothrombin | P | GO: 0048731 | system development |
| | os = homo sapiens gn = f2 | | | |
| | pe = 1 sv = 2 | | | |
| gi\|194383496 | thrb_humanprothrombin | C | GO: 0016020 | membrane |
| | os = homo sapiens gn = f2 | | | |
| | pe = 1 sv = 2 | | | |
| gi\|194383496 | thrb_humanprothrombin | P | GO: 0051480 | cytosolic calcium ion |
| | os = homo sapiens gn = f2 | | | homeostasis |
| | pe = 1 sv = 2 | | | |
| KH 6 | 28071026 | Fr III | gi\|28071026 | ighm_humanig mu chain c |
| | | | | region os = homo sapiens |
| | | | | gn = ighm pe = 1 sv = 3 |
| gi\|28071026 | ighm_humanig mu chain c | F | GO: 0005488 | binding |
| | region os = homo sapiens | | | |
| | gn = ighm pe = 1 sv = 3 | | | |
| gi\|28071026 | ighm_humanig mu chain c | C | GO: 0044464 | cell part |
| | region os = homo sapiens | | | |
| | gn = ighm pe = 1 sv = 3 | | | |
| gi\|28071026 | ighm_humanig mu chain c | C | GO: 0016020 | membrane |
| | region os = homo sapiens | | | |
| | gn = ighm pe = 1 sv = 3 | | | |
| gi\|28071026 | ighm_humanig mu chain c | P | GO: 0006955 | immune response |
| | region os = homo sapiens | | | |
| | gn = ighm pe = 1 sv = 3 | | | |
| KH 7 | 300621695 | Fr III | gi\|300621695 | ighm_humanig mu chain c |
| | | | | region os = homo sapiens |
| | | | | gn = ighm pe = 1 sv = 3 |
| gi\|300621695 | ighm_humanig mu chain c | P | GO: 0006955 | immune response |
| | region os = homo sapiens | | | |
| | gn = ighm pe = 1 sv = 3 | | | |
| KH 8 | 1335098 | Fr III | gi\|1335098 | hemo_humanhemopexin |
| | | | | os = homo sapiens gn = hpx |
| | | | | pe = 1 sv = 2 |
| gi\|1335098 | hemo_humanhemopexin | P | GO: 0008152 | metabolic process |
| | os = homo sapiens gn = hpx | | | |
| | pe = 1 sv = 2 | | | |
| gi\|1335098 | hemo_humanhemopexin | P | GO: 0051179 | localization |
| | os = homo sapiens gn = hpx | | | |
| | pe = 1 sv = 2 | | | |
| gi\|1335098 | hemo_humanhemopexin | C | GO: 0005615 | extracellular space |
| | os = homo sapiens gn = hpx | | | |
| | pe = 1 sv = 2 | | | |
| gi\|1335098 | hemo_humanhemopexin | F | GO: 0005515 | protein binding |
| | os = homo sapiens gn = hpx | | | |
| | pe = 1 sv = 2 | | | |
| gi\|1335098 | hemo_humanhemopexin | P | GO: 0048522 | positive regulation of |
| | os = homo sapiens gn = hpx | | | cellular process |
| | pe = 1 sv = 2 | | | |
| gi\|1335098 | hemo_humanhemopexin | P | GO: 0050896 | response to stimulus |
| | os = homo sapiens gn = hpx | | | |
| | pe = 1 sv = 2 | | | |
| KH 9 | 10434804 | Fr III | gi\|10434804 | mthsd_humanmethenyltetrahydrofolate |
| | | | | synthase domain- |
| | | | | containing protein os = homo |
| | | | | sapiens gn = mthfsd pe = 1 |
| | | | | sv = 2 |
| gi\|10434804 | mthsd_humanmethenyltetrahydrofolate | F | GO: 0005524 | ATP binding |
| | synthase domain- | | | |
| | containing protein os = homo | | | |
| | sapiens gn = mthfsd pe = 1 | | | |
| | sv = 2 | | | |
| gi\|10434804 | mthsd_humanmethenyltetrahydrofolate | P | GO: 0009396 | folic acid-containing |
| | synthase domain- | | | compound biosynthetic |
| | containing protein os = homo | | | process |
| | sapiens gn = mthfsd pe = 1 | | | |
| | sv = 2 | | | |
| gi\|10434804 | mthsd_humanmethenyltetrahydrofolate | F | GO: 0030272 | 5-formyltetrahydrofolate |
| | synthase domain- | | | cyclo-ligase activity |
| | containing protein os = homo | | | |
| | sapiens gn = mthfsd pe = 1 | | | |
| | sv = 2 | | | |
| KH 10 | 221044726 | Fr III | gi\|221044726 | hemo_humanhemopexin |
| | | | | os = homo sapiens gn = hpx |
| | | | | pe = 1 sv = 2 |
| gi\|221044726 | hemo_humanhemopexin | F | GO: 0005515 | protein binding |
| | os = homo sapiens gn = hpx | | | |
| | pe = 1 sv = 2 | | | |
| gi\|221044726 | hemo_humanhemopexin | C | GO: 0005615 | extracellular space |
| | os = homo sapiens gn = hpx | | | |
| | pe = 1 sv = 2 | | | |
| gi\|221044726 | hemo_humanhemopexin | P | GO: 0009987 | cellular process |
| | os = homo sapiens gn = hpx | | | |
| | pe = 1 sv = 2 | | | |
| gi\|221044726 | hemo_humanhemopexin | P | GO: 0065007 | biological regulation |
| | os = homo sapiens gn = hpx | | | |
| | pe = 1 sv = 2 | | | |
| KH 11 | 215415638 | PCC | same as | |
| | | | KH 3 | |
| KH 12 | 189066554 | PCC | gi\|189066554 | thrb_humanprothrombin |
| | | | | os = homo sapiens gn = f2 |
| | | | | pe = 1 sv = 2 |
| gi\|189066554 | thrb_humanprothrombin | C | GO: 0044446 | intracellular organelle part |
| | os = homo sapiens gn = f2 | | | |
| | pe = 1 sv = 2 | | | |
| gi\|189066554 | thrb_humanprothrombin | P | GO: 0048712 | negative regulation of |
| | os = homo sapiens gn = f2 | | | astrocyte differentiation |
| | pe = 1 sv = 2 | | | |
| gi\|189066554 | thrb_humanprothrombin | C | GO: 0043233 | organelle lumen |
| | os = homo sapiens gn = f2 | | | |
| | pe = 1 sv = 2 | | | |
| gi\|189066554 | thrb_humanprothrombin | P | GO: 0030194 | positive regulation of blood |
| | os = homo sapiens gn = f2 | | | coagulation |
| | pe = 1 sv = 2 | | | |
| gi\|189066554 | thrb_humanprothrombin | F | GO: 0005102 | receptor binding |
| | os = homo sapiens gn = f2 | | | |
| | pe = 1 sv = 2 | | | |
| gi\|189066554 | thrb_humanprothrombin | P | GO: 2000379 | positive regulation of |
| | os = homo sapiens gn = f2 | | | reactive oxygen species |
| | pe = 1 sv = 2 | | | metabolic process |
| gi\|189066554 | thrb_humanprothrombin | P | GO: 0045861 | negative regulation of |
| | os = homo sapiens gn = f2 | | | proteolysis |
| | pe = 1 sv = 2 | | | |
| gi\|189066554 | thrb_humanprothrombin | C | GO: 0005615 | extracellular space |
| | os = homo sapiens gn = f2 | | | |
| | pe = 1 sv = 2 | | | |
| gi\|189066554 | thrb_humanprothrombin | P | GO: 0030168 | platelet activation |
| | os = homo sapiens gn = f2 | | | |
| | pe = 1 sv = 2 | | | |
| gi\|189066554 | thrb_humanprothrombin | P | GO: 1900738 | positive regulation of |
| | os = homo sapiens gn = f2 | | | phospholipase C-activating |
| | pe = 1 sv = 2 | | | G-protein coupled receptor |
| | | | | signaling pathway |
| gi\|189066554 | thrb_humanprothrombin | P | GO: 0016477 | cell migration |
| | os = homo sapiens gn = f2 | | | |
| | pe = 1 sv = 2 | | | |
| gi\|189066554 | thrb_humanprothrombin | C | GO: 0043231 | intracellular membrane- |
| | os = homo sapiens gn = f2 | | | bounded organelle |
| | pe = 1 sv = 2 | | | |
| gi\|189066554 | thrb_humanprothrombin | P | GO: 0001934 | positive regulation of protein |
| | os = homo sapiens gn = f2 | | | phosphorylation |
| | pe = 1 sv = 2 | | | |
| gi\|189066554 | thrb_humanprothrombin | C | GO: 0005886 | plasma membrane |
| | os = homo sapiens gn = f2 | | | |
| | pe = 1 sv = 2 | | | |
| gi\|189066554 | thrb_humanprothrombin | F | GO: 0070053 | thrombospondin receptor |
| | os = homo sapiens gn = f2 | | | activity |
| | pe = 1 sv = 2 | | | |
| gi\|189066554 | thrb_humanprothrombin | P | GO: 0051281 | positive regulation of release |
| | os = homo sapiens gn = f2 | | | of sequestered calcium ion |
| | pe = 1 sv = 2 | | | into cytosol |
| gi\|189066554 | thrb_humanprothrombin | F | GO: 0004252 | serine-type endopeptidase |
| | os = homo sapiens gn = f2 | | | activity |
| | pe = 1 sv = 2 | | | |
| gi\|189066554 | thrb_humanprothrombin | P | GO: 0042730 | fibrinolysis |
| | os = homo sapiens gn = f2 | | | |
| | pe = 1 sv = 2 | | | |
| gi\|189066554 | thrb_humanprothrombin | C | GO: 0044444 | cytoplasmic part |
| | os = homo sapiens gn = f2 | | | |
| | pe = 1 sv = 2 | | | |
| gi\|189066554 | thrb_humanprothrombin | P | GO: 0032967 | positive regulation of |
| | os = homo sapiens gn = f2 | | | collagen biosynthetic |
| | pe = 1 sv = 2 | | | process |
| KH 13 | 194391084 | PCC | gi\|194391084 | kng1_humankininogen-1 |
| | | | | os = homo sapiens gn = kng1 |
| | | | | pe = 1 sv = 2 |
| gi\|194391084 | kng1_humankininogen-1 | F | GO: 0005515 | protein binding |
| | os = homo sapiens gn = kng1 | | | |
| | pe = 1 sv = 2 | | | |
| gi\|194391084 | kng1_humankininogen-1 | P | GO: 0055065 | metal ion homeostasis |
| | os = homo sapiens gn = kng1 | | | |
| | pe = 1 sv = 2 | | | |
| gi\|194391084 | kng1_humankininogen-1 | P | GO: 0051241 | negative regulation of |
| | os = homo sapiens gn = kng1 | | | multicellular organismal |
| | pe = 1 sv = 2 | | | process |
| gi\|194391084 | kng1_humankininogen-1 | P | GO: 0007596 | blood coagulation |
| | os = homo sapiens gn = kng1 | | | |
| | pe = 1 sv = 2 | | | |
| gi\|194391084 | kng1_humankininogen-1 | C | GO: 0043229 | intracellular organelle |
| | os = homo sapiens gn = kng1 | | | |
| | pe = 1 sv = 2 | | | |
| gi\|194391084 | kng1_humankininogen-1 | P | GO: 0048523 | negative regulation of |
| | os = homo sapiens gn = kng1 | | | cellular process |
| | pe = 1 sv = 2 | | | |
| gi\|194391084 | kng1_humankininogen-1 | P | GO: 0008152 | metabolic process |
| | os = homo sapiens gn = kng1 | | | |
| | pe = 1 sv = 2 | | | |
| gi\|194391084 | kng1_humankininogen-1 | P | GO: 0003008 | system process |
| | os = homo sapiens gn = kng1 | | | |
| | pe = 1 sv = 2 | | | |
| KH 14 | 158255114 | PCC | gi\|158255114 | kng1_humankininogen-1 |
| | | | | os = homo sapiens gn = kng1 |
| | | | | pe = 1 sv = 2 |
| gi\|158255114 | kng1_humankininogen-1 | F | GO: 0005515 | protein binding |
| | os = homo sapiens gn = kng1 | | | |
| | pe = 1 sv = 2 | | | |
| gi\|158255114 | kng1_humankininogen-1 | P | GO: 0055065 | metal ion homeostasis |
| | os = homo sapiens gn = kng1 | | | |
| | pe = 1 sv = 2 | | | |
| gi\|158255114 | kng1_humankininogen-1 | P | GO: 0051241 | negative regulation of |
| | os = homo sapiens gn = kng1 | | | multicellular organismal |
| | pe = 1 sv = 2 | | | process |
| gi\|158255114 | kng1_humankininogen-1 | P | GO: 0007596 | blood coagulation |
| | os = homo sapiens gn = kng1 | | | |
| | pe = 1 sv = 2 | | | |
| gi\|158255114 | kng1_humankininogen-1 | C | GO: 0043229 | intracellular organelle |
| | os = homo sapiens gn = kng1 | | | |
| | pe = 1 sv = 2 | | | |
| gi\|158255114 | kng1_humankininogen-1 | P | GO: 0048523 | negative regulation of |
| | os = homo sapiens gn = kng1 | | | cellular process |
| | pe = 1 sv = 2 | | | |
| gi\|158255114 | kng1_humankininogen-1 | P | GO: 0008152 | metabolic process |
| | os = homo sapiens gn = kng1 | | | |
| | pe = 1 sv = 2 | | | |
| gi\|158255114 | kng1_humankininogen-1 | P | GO: 0003008 | system process |
| | os = homo sapiens gn = kng1 | | | |
| | pe = 1 sv = 2 | | | |
| KH 15 | 213506121 | PCC | gi\|213506121 | kng1_humankininogen-1 |
| | | | | os = homo sapiens gn = kng1 |
| | | | | pe = 1 sv = 2 |
| gi\|213506121 | kng1_humankininogen-1 | F | GO: 0005515 | protein binding |
| | os = homo sapiens gn = kng1 | | | |
| | pe = 1 sv = 2 | | | |
| gi\|213506121 | kng1_humankininogen-1 | P | GO: 0055065 | metal ion homeostasis |
| | os = homo sapiens gn = kng1 | | | |
| | pe = 1 sv = 2 | | | |
| gi\|213506121 | kng1_humankininogen-1 | P | GO: 0051241 | negative regulation of |
| | os = homo sapiens gn = kng1 | | | multicellular organismal |
| | pe = 1 sv = 2 | | | process |
| gi\|213506121 | kng1_humankininogen-1 | P | GO: 0007596 | blood coagulation |
| | os = homo sapiens gn = kng1 | | | |
| | pe = 1 sv = 2 | | | |
| gi\|213506121 | kng1_humankininogen-1 | C | GO: 0043229 | intracellular organelle |
| | os = homo sapiens gn = kng1 | | | |
| | pe = 1 sv = 2 | | | |
| gi\|213506121 | kng1_humankininogen-1 | P | GO: 0048523 | negative regulation of |
| | os = homo sapiens gn = kng1 | | | cellular process |
| | pe = 1 sv = 2 | | | |
| gi\|213506121 | kng1_humankininogen-1 | P | GO: 0008152 | metabolic process |
| | os = homo sapiens gn = kng1 | | | |
| | pe = 1 sv = 2 | | | |
| gi\|213506121 | kng1_humankininogen-1 | P | GO: 0003008 | system process |
| | os = homo sapiens gn = kng1 | | | |
| | pe = 1 sv = 2 | | | |
| KH 16 | 213506103 | PCC | gi\|213506103 | kng1_humankininogen-1 |
| | | | | os = homo sapiens gn = kng1 |
| | | | | pe = 1 sv = 2 |
| gi\|213506103 | kng1_humankininogen-1 | F | GO: 0005515 | protein binding |
| | os = homo sapiens gn = kng1 | | | |
| | pe = 1 sv = 2 | | | |
| gi\|213506103 | kng1_humankininogen-1 | P | GO: 0055065 | metal ion homeostasis |
| | os = homo sapiens gn = kng1 | | | |
| | pe = 1 sv = 2 | | | |
| gi\|213506103 | kng1_humankininogen-1 | P | GO: 0051241 | negative regulation of |
| | os = homo sapiens gn = kng1 | | | multicellular organismal |
| | pe = 1 sv = 2 | | | process |
| gi\|213506103 | kng1_humankininogen-1 | P | GO: 0007596 | blood coagulation |
| | os = homo sapiens gn = kng1 | | | |
| | pe = 1 sv = 2 | | | |
| gi\|213506103 | kng1_humankininogen-1 | C | GO: 0043229 | intracellular organelle |
| | os = homo sapiens gn = kng1 | | | |
| | pe = 1 sv = 2 | | | |
| gi\|213506103 | kng1_humankininogen-1 | P | GO: 0048523 | negative regulation of |
| | os = homo sapiens gn = kng1 | | | cellular process |
| | pe = 1 sv = 2 | | | |
| gi\|213506103 | kng1_humankininogen-1 | P | GO: 0008152 | metabolic process |
| | os = homo sapiens gn = kng1 | | | |
| | pe = 1 sv = 2 | | | |
| gi\|213506103 | kng1_humankininogen-1 | P | GO: 0003008 | system process |
| | os = homo sapiens gn = kng1 | | | |
| | pe = 1 sv = 2 | | | |
| KH 17 | 194376310 | PCC | gi\|194376310 | cytoplasmic 1 os = homo |
| | | | | sapiens gn = actb pe = 1 sv = 1 |
| gi\|194376310 | cytoplasmic 1 os = homo | P | GO: 0009888 | tissue development |
| | sapiens gn = actb pe = 1 sv = 1 | | | |
| gi\|194376310 | cytoplasmic 1 os = homo | P | GO: 0030048 | actin filament-based |
| | sapiens gn = actb pe = 1 sv = 1 | | | movement |
| gi\|194376310 | cytoplasmic 1 os = homo | P | GO: 0003012 | muscle system process |
| | sapiens gn = actb pe = 1 sv = 1 | | | |
| gi\|194376310 | cytoplasmic 1 os = homo | C | GO: 0030017 | sarcomere |
| | sapiens gn = actb pe = 1 sv = 1 | | | |
| gi\|194376310 | cytoplasmic 1 os = homo | P | GO: 0030239 | myofibril assembly |
| | sapiens gn = actb pe = 1 sv = 1 | | | |
| gi\|194376310 | cytoplasmic 1 os = homo | P | GO: 0044238 | primary metabolic process |
| | sapiens gn = actb pe = 1 sv = 1 | | | |
| gi\|194376310 | cytoplasmic 1 os = homo | C | GO: 0005884 | actin filament |
| | sapiens gn = actb pe = 1 sv = 1 | | | |
| gi\|194376310 | cytoplasmic 1 os = homo | P | GO: 0072358 | cardiovascular system |
| | sapiens gn = actb pe = 1 sv = 1 | | | development |
| gi\|194376310 | cytoplasmic 1 os = homo | P | GO: 0044237 | cellular metabolic process |
| | sapiens gn = actb pe = 1 sv = 1 | | | |
| gi\|194376310 | cytoplasmic 1 os = homo | P | GO: 0048513 | organ development |
| | sapiens gn = actb pe = 1 sv = 1 | | | |
| gi\|194376310 | cytoplasmic 1 os = homo | F | GO: 0005515 | protein binding |
| | sapiens gn = actb pe = 1 sv = 1 | | | |
| gi\|194376310 | cytoplasmic 1 os = homo | P | GO: 0042221 | response to chemical |
| | sapiens gn = actb pe = 1 sv = 1 | | | stimulus |
| gi\|194376310 | cytoplasmic 1 os = homo | P | GO: 0008015 | blood circulation |
| | sapiens gn = actb pe = 1 sv = 1 | | | |
| KH 18 | 194388064 | PCC | gi\|194388064 | cytoplasmic 2 os = homo |
| | | | | sapiens gn = actg1 pe = 1 sv = 1 |
| gi\|194388064 | cytoplasmic 2 os = homo | P | GO: 0009888 | tissue development |
| | sapiens gn = actg1 pe = 1 sv = 1 | | | |
| gi\|194388064 | cytoplasmic 2 os = homo | P | GO: 0030048 | actin filament-based |
| | sapiens gn = actg1 pe = 1 sv = 1 | | | movement |
| gi\|194388064 | cytoplasmic 2 os = homo | P | GO: 0003012 | muscle system process |
| | sapiens gn = actg1 pe = 1 sv = 1 | | | |
| gi\|194388064 | cytoplasmic 2 os = homo | C | GO: 0030017 | sarcomere |
| | sapiens gn = actg1 pe = 1 sv = 1 | | | |
| gi\|194388064 | cytoplasmic 2 os = homo | P | GO: 0030239 | myofibril assembly |
| | sapiens gn = actg1 pe = 1 sv = 1 | | | |
| gi\|194388064 | cytoplasmic 2 os = homo | P | GO: 0044238 | primary metabolic process |
| | sapiens gn = actg1 pe = 1 sv = 1 | | | |
| gi\|194388064 | cytoplasmic 2 os = homo | C | GO: 0005884 | actin filament |
| | sapiens gn = actg1 pe = 1 sv = 1 | | | |
| gi\|194388064 | cytoplasmic 2 os = homo | P | GO: 0072358 | cardiovascular system |
| | sapiens gn = actg1 pe = 1 sv = 1 | | | development |
| gi\|194388064 | cytoplasmic 2 os = homo | P | GO: 0044237 | cellular metabolic process |
| | sapiens gn = actg1 pe = 1 sv = 1 | | | |
| gi\|194388064 | cytoplasmic 2 os = homo | P | GO: 0048513 | organ development |
| | sapiens gn = actg1 pe = 1 sv = 1 | | | |
| gi\|194388064 | cytoplasmic 2 os = homo | P | GO: 0042221 | response to chemical |
| | sapiens gn = actg1 pe = 1 sv = 1 | | | stimulus |
| gi\|194388064 | cytoplasmic 2 os = homo | F | GO: 0008092 | cytoskeletal protein binding |
| | sapiens gn = actg1 pe = 1 sv = 1 | | | |
| gi\|194388064 | cytoplasmic 2 os = homo | P | GO: 0065008 | regulation of biological |
| | sapiens gn = actg1 pe = 1 sv = 1 | | | quality |
| gi\|194388064 | cytoplasmic 2 os = homo | C | GO: 0044451 | nucleoplasm part |
| | sapiens gn = actg1 pe = 1 sv = 1 | | | |
| gi\|194388064 | cytoplasmic 2 os = homo | P | GO: 0008015 | blood circulation |
| | sapiens gn = actg1 pe = 1 sv = 1 | | | |
| gi\|194388064 | cytoplasmic 2 os = homo | F | GO: 0019899 | enzyme binding |
| | sapiens gn = actg1 pe = 1 sv = 1 | | | |
| gi\|194391084 | kng1_humankininogen-1 | F | GO: 0005515 | protein binding |
| | os = homo sapiens gn = kng1 | | | |
| | pe = 1 sv = 2 | | | |
| gi\|194391084 | kng1_humankininogen-1 | P | GO: 0055065 | metal ion homeostasis |
| | os = homo sapiens gn = kng1 | | | |
| | pe = 1 sv = 2 | | | |
| gi\|194391084 | kng1_humankininogen-1 | P | GO: 0051241 | negative regulation of |
| | os = homo sapiens gn = kng1 | | | multicellular organismal |
| | pe = 1 sv = 2 | | | process |
| gi\|194391084 | kng1_humankininogen-1 | P | GO: 0007596 | blood coagulation |
| | os = homo sapiens gn = kng1 | | | |
| | pe = 1 sv = 2 | | | |
| gi\|194391084 | kng1_humankininogen-1 | C | GO: 0043229 | intracellular organelle |
| | os = homo sapiens gn = kng1 | | | |
| | pe = 1 sv = 2 | | | |
| gi\|194391084 | kng1_humankininogen-1 | P | GO: 0048523 | negative regulation of |
| | os = homo sapiens gn = kng1 | | | cellular process |
| | pe = 1 sv = 2 | | | |
| gi\|194391084 | kng1_humankininogen-1 | P | GO: 0008152 | metabolic process |
| | os = homo sapiens gn = kng1 | | | |
| | pe = 1 sv = 2 | | | |
| gi\|194391084 | kng1_humankininogen-1 | P | GO: 0003008 | system process |
| | os = homo sapiens gn = kng1 | | | |
| | pe = 1 sv = 2 | | | |
| KH 19 | IPI00964149 | AFCC | IPI00964149 | pacrl_humanpacrg-like |
| | | | | protein os = homo sapiens |
| | | | | gn = pacrgl pe = 1 sv = 2 |
| KH 20 | IPI00966721 | AFCC | IPI00966721 | ce028_humantransmembrane |
| | | | | protein c5orf28 os = homo |
| | | | | sapiens gn = c5orf28 pe = 2 |
| | | | | sv = 1 |
| IPI00966721 | ce028_humantransmembrane | C | GO: 0016021 | integral to membrane |
| | protein c5orf28 os = homo | | | |
| | sapiens gn = c5orf28 pe = 2 | | | |
| | sv = 1 | | | |
| KH 21 | IPI00966826 | FrIV | IPI00966826 | pds5a_humansister |
| | | | | chromatid cohesion protein |
| | | | | pds5 homolog a os = homo |
| | | | | sapiens gn = pds5a pe = 1 sv = 1 |
| IPI00966826 | pds5a_humansister | P | GO: 0008156 | negative regulation of DNA |
| | chromatid cohesion protein | | | replication |
| | pds5 homolog a os = homo | | | |
| | sapiens gn = pds5a pe = 1 sv = 1 | | | |
| IPI00966826 | pds5a_humansister | C | GO: 0005730 | nucleolus |
| | chromatid cohesion protein | | | |
| | pds5 homolog a os = homo | | | |
| | sapiens gn = pds5a pe = 1 sv = 1 | | | |
| IPI00966826 | pds5a_humansister | C | GO: 0000785 | chromatin |
| | chromatid cohesion protein | | | |
| | pds5 homolog a os = homo | | | |
| | sapiens gn = pds5a pe = 1 sv = 1 | | | |
| IPI00966826 | pds5a_humansister | F | GO: 0005515 | protein binding |
| | chromatid cohesion protein | | | |
| | pds5 homolog a os = homo | | | |
| | sapiens gn = pds5a pe = 1 sv = 1 | | | |
| IPI00966826 | pds5a_humansister | P | GO: 0008283 | cell proliferation |
| | chromatid cohesion protein | | | |
| | pds5 homolog a os = homo | | | |
| | sapiens gn = pds5a pe = 1 sv = 1 | | | |
| IPI00966826 | pds5a_humansister | C | GO: 0005886 | plasma membrane |
| | chromatid cohesion protein | | | |
| | pds5 homolog a os = homo | | | |
| | sapiens gn = pds5a pe = 1 sv = 1 | | | |
| KH 22 | IPI00760788 | FrIV | IPI00760788 | klh22_humankelch-like |
| | | | | protein 22 os = homo sapiens |
| | | | | gn = klhl22 pe = 1 sv = 2 |
| IPI00760788 | klh22_humankelch-like | P | GO: 0051301 | cell division |
| | protein 22 os = homo sapiens | | | |
| | gn = klhl22 pe = 1 sv = 2 | | | |
| IPI00760788 | klh22_humankelch-like | C | GO: 0031463 | Cul3-RING ubiquitin ligase |
| | protein 22 os = homo sapiens | | | complex |
| | gn = klhl22 pe = 1 sv = 2 | | | |
| KH 23 | IPI00917278 | FrIV | | |
| KH 24 | IPI00966721 | AFOD | same as | |
| | | | KH 20 | |
| KH 25 | IPI01012037 | AFOD | IPI01012037 | mcm8_humandna helicase |
| | | | | mcm8 os = homo sapiens |
| | | | | gn = mcm8 pe = 1 sv = 2 |
| IPI01012037 | mcm8_humandna helicase | P | GO: 0051329 | interphase of mitotic cell |
| | mcm8 os = homo sapiens | | | cycle |
| | gn = mcm8 pe = 1 sv = 2 | | | |
| IPI01012037 | mcm8_humandna helicase | P | GO: 0034645 | cellular macromolecule |
| | mcm8 os = homo sapiens | | | biosynthetic process |
| | gn = mcm8 pe = 1 sv = 2 | | | |
| IPI01012037 | mcm8_humandna helicase | P | GO: 0090304 | nucleic acid metabolic |
| | mcm8 os = homo sapiens | | | process |
| | gn = mcm8 pe = 1 sv = 2 | | | |
| KH 26 | IPI00940730 | AFOD | IPI00940730 | enoph_humanenolase- |
| | | | | phosphatase e1 os = homo |
| | | | | sapiens gn = enoph1 pe = 1 |
| | | | | sv = 1 |
| IPI00940730 | enoph_humanenolase- | P | GO: 0019509 | L-methionine salvage from |
| | phosphatase e1 os = homo | | | methylthioadenosine |
| | sapiens gn = enoph1 pe = 1 | | | |
| | sv = 1 | | | |
| IPI00940730 | enoph_humanenolase- | F | GO: 0043874 | acireductone synthase |
| | phosphatase e1 os = homo | | | activity |
| | sapiens gn = enoph1 pe = 1 | | | |
| | sv = 1 | | | |
| KH 27 | IPI00977191 | AFOD | | |
| KH 28 | IPI00022434 | HemoRAAS | IPI00022434 | albu_humanserum albumin |
| | | | | os = homo sapiens gn = alb |
| | | | | pe = 1 sv = 2 |
| IPI00022434 | albu_humanserum albumin | P | GO: 0008202 | steroid metabolic process |
| | os = homo sapiens gn = alb | | | |
| | pe = 1 sv = 2 | | | |
| IPI00022434 | albu_humanserum albumin | P | GO: 0051704 | multi-organism process |
| | os = homo sapiens gn = alb | | | |
| | pe = 1 sv = 2 | | | |
| IPI00022434 | albu_humanserum albumin | C | GO: 0044446 | intracellular organelle part |
| | os = homo sapiens gn = alb | | | |
| | pe = 1 sv = 2 | | | |
| IPI00022434 | albu_humanserum albumin | P | GO: 0051641 | cellular localization |
| | os = homo sapiens gn = alb | | | |
| | pe = 1 sv = 2 | | | |
| IPI00022434 | albu_humanserum albumin | P | GO: 0051716 | cellular response to stimulus |
| | os = homo sapiens gn = alb | | | |
| | pe = 1 sv = 2 | | | |
| IPI00022434 | albu_humanserum albumin | F | GO: 0008289 | lipid binding |
| | os = homo sapiens gn = alb | | | |
| | pe = 1 sv = 2 | | | |
| IPI00022434 | albu_humanserum albumin | P | GO: 0043069 | negative regulation of |
| | os = homo sapiens gn = alb | | | programmed cell death |
| | pe = 1 sv = 2 | | | |
| IPI00022434 | albu_humanserum albumin | P | GO: 0044260 | cellular macromolecule |
| | os = homo sapiens gn = alb | | | metabolic process |
| | pe = 1 sv = 2 | | | |
| IPI00022434 | albu_humanserum albumin | P | GO: 0031667 | response to nutrient levels |
| | os = homo sapiens gn = alb | | | |
| | pe = 1 sv = 2 | | | |
| IPI00022434 | albu_humanserum albumin | C | GO: 0043231 | intracellular membrane- |
| | os = homo sapiens gn = alb | | | bounded organelle |
| | pe = 1 sv = 2 | | | |
| IPI00022434 | albu_humanserum albumin | P | GO: 0044281 | small molecule metabolic |
| | os = homo sapiens gn = alb | | | process |
| | pe = 1 sv = 2 | | | |
| IPI00022434 | albu_humanserum albumin | F | GO: 0005515 | protein binding |
| | os = homo sapiens gn = alb | | | |
| | pe = 1 sv = 2 | | | |
| IPI00022434 | albu_humanserum albumin | P | GO: 0006810 | transport |
| | os = homo sapiens gn = alb | | | |
| | pe = 1 sv = 2 | | | |
| IPI00022434 | albu_humanserum albumin | P | GO: 0065008 | regulation of biological |
| | os = homo sapiens gn = alb | | | quality |
| | pe = 1 sv = 2 | | | |
| IPI00022434 | albu_humanserum albumin | P | GO: 0007154 | cell communication |
| | os = homo sapiens gn = alb | | | |
| | pe = 1 sv = 2 | | | |
| IPI00022434 | albu_humanserum albumin | F | GO: 0019842 | vitamin binding |
| | os = homo sapiens gn = alb | | | |
| | pe = 1 sv = 2 | | | |
| IPI00022434 | albu_humanserum albumin | P | GO: 0006950 | response to stress |
| | os = homo sapiens gn = alb | | | |
| | pe = 1 sv = 2 | | | |
| IPI00022434 | albu_humanserum albumin | C | GO: 0044444 | cytoplasmic part |
| | os = homo sapiens gn = alb | | | |
| | pe = 1 sv = 2 | | | |
| IPI00022434 | albu_humanserum albumin | P | GO: 0032501 | multicellular organismal |
| | os = homo sapiens gn = alb | | | process |
| | pe = 1 sv = 2 | | | |
| IPI00022434 | albu_humanserum albumin | C | GO: 0044421 | extracellular region part |
| | os = homo sapiens gn = alb | | | |
| | pe = 1 sv = 2 | | | |
| KH 29 | IPI00022434 | HemoRAAS | same as | |
| | | | KH 28 | |
| KH 30 | IPI00219713 | FibroRAAS | IPI00219713 | fibg_humanfibrinogen |
| | | | | gamma chain os = homo |
| | | | | sapiens gn = fgg pe = 1 sv = 3 |
| IPI00219713 | fibg_humanfibrinogen | P | GO: 0009987 | cellular process |
| | gamma chain os = homo | | | |
| | sapiens gn = fgg pe = 1 sv = 3 | | | |
| IPI00219713 | fibg_humanfibrinogen | C | GO: 0009897 | external side of plasma |
| | gamma chain os = homo | | | membrane |
| | sapiens gn = fgg pe = 1 sv = 3 | | | |
| IPI00219713 | fibg_humanfibrinogen | F | GO: 0043499 | eukaryotic cell surface |
| | gamma chain os = homo | | | binding |
| | sapiens gn = fgg pe = 1 sv = 3 | | | |
| IPI00219713 | fibg_humanfibrinogen | C | GO: 0005615 | extracellular space |
| | gamma chain os = homo | | | |
| | sapiens gn = fgg pe = 1 sv = 3 | | | |
| IPI00219713 | fibg_humanfibrinogen | C | GO: 0031091 | platelet alpha granule |
| | gamma chain os = homo | | | |
| | sapiens gn = fgg pe = 1 sv = 3 | | | |
| IPI00219713 | fibg_humanfibrinogen | P | GO: 0032501 | multicellular organismal |
| | gamma chain os = homo | | | process |
| | sapiens gn = fgg pe = 1 sv = 3 | | | |
| IPI00219713 | fibg_humanfibrinogen | P | GO: 0065007 | biological regulation |
| | gamma chain os = homo | | | |
| | sapiens gn = fgg pe = 1 sv = 3 | | | |
| IPI00219713 | fibg_humanfibrinogen | P | GO: 0051592 | response to calcium ion |
| | gamma chain os = homo | | | |
| | sapiens gn = fgg pe = 1 sv = 3 | | | |
| KH 31 | IPI00219713 | FibroRAAS | same as | |
| | | | KH 30 | |
| KH 32 | IPI00220327 | FibroRAAS | IPI00220327 | type ii cytoskeletal 1 |
| | | | | os = homo sapiens gn = krt1 |
| | | | | pe = 1 sv = 6 |
| IPI00220327 | type ii cytoskeletal 1 | P | GO: 0009987 | cellular process |
| | os = homo sapiens gn = krt1 | | | |
| | pe = 1 sv = 6 | | | |
| IPI00220327 | type ii cytoskeletal 1 | P | GO: 0048731 | system development |
| | os = homo sapiens gn = krt1 | | | |
| | pe = 1 sv = 6 | | | |
| IPI00220327 | type ii cytoskeletal 1 | P | GO: 0009888 | tissue development |
| | os = homo sapiens gn = krt1 | | | |
| | pe = 1 sv = 6 | | | |
| IPI00220327 | type ii cytoskeletal 1 | C | GO: 0005856 | cytoskeleton |
| | os = homo sapiens gn = krt1 | | | |
| | pe = 1 sv = 6 | | | |
| IPI00220327 | type ii cytoskeletal 1 | F | GO: 0005515 | protein binding |
| | os = homo sapiens gn = krt1 | | | |
| | pe = 1 sv = 6 | | | |
| IPI00220327 | type ii cytoskeletal 1 | P | GO: 0001867 | complement activation, |
| | os = homo sapiens gn = krt1 | | | lectin pathway |
| | pe = 1 sv = 6 | | | |
| IPI00220327 | type ii cytoskeletal 1 | F | GO: 0030246 | carbohydrate binding |
| | os = homo sapiens gn = krt1 | | | |
| | pe = 1 sv = 6 | | | |
| IPI00220327 | type ii cytoskeletal 1 | C | GO: 0016020 | membrane |
| | os = homo sapiens gn = krt1 | | | |
| | pe = 1 sv = 6 | | | |
| KH 33 | IPI00029739 | GammaRAAS | IPI00029739 | cfah_humancomplement |
| | | | | factor h os = homo sapiens |
| | | | | gn = cfh pe = 1 sv = 4 |
| IPI00029739 | cfah_humancomplement | P | GO: 0030449 | regulation of complement |
| | factor h os = homo sapiens | | | activation |
| | gn = cfh pe = 1 sv = 4 | | | |
| IPI00029739 | cfah_humancomplement | P | GO: 0045087 | innate immune response |
| | factor h os = homo sapiens | | | |
| | gn = cfh pe = 1 sv = 4 | | | |
| KH 34 | IPI00384853 | GammaRAAS | | |
| KH 35 | IPI00479708 | GammaRAAS | IPI00479708 | ighm_humanig mu chain c |
| | | | | region os = homo sapiens |
| | | | | gn = ighm pe = 1 sv = 3 |
| IPI00479708 | ighm_humanig mu chain c | F | GO: 0005488 | binding |
| | region os = homo sapiens | | | |
| | gn = ighm pe = 1 sv = 3 | | | |
| IPI00479708 | ighm_humanig mu chain c | C | GO: 0044464 | cell part |
| | region os = homo sapiens | | | |
| | gn = ighm pe = 1 sv = 3 | | | |
| IPI00479708 | ighm_humanig mu chain c | C | GO: 0016020 | membrane |
| | region os = homo sapiens | | | |
| | gn = ighm pe = 1 sv = 3 | | | |
| IPI00479708 | ighm_humanig mu chain c | P | GO: 0006955 | immune response |
| | region os = homo sapiens | | | |
| | gn = ighm pe = 1 sv = 3 | | | |
| KH 36 | IPI00298497 | GammaRAAS | IPI00298497 | fibb_humanfibrinogen beta |
| | | | | chain os = homo sapiens |
| | | | | gn = fgb pe = 1 sv = 2 |
| IPI00298497 | fibb_humanfibrinogen beta | F | GO: 0051087 | chaperone binding |
| | chain os = homo sapiens | | | |
| | gn = fgb pe = 1 sv = 2 | | | |
| IPI00298497 | fibb_humanfibrinogen beta | P | GO: 0051592 | response to calcium ion |
| | chain os = homo sapiens | | | |
| | gn = fgb pe = 1 sv = 2 | | | |
| IPI00298497 | fibb_humanfibrinogen beta | C | GO: 0005615 | extracellular space |
| | chain os = homo sapiens | | | |
| | gn = fgb pe = 1 sv = 2 | | | |
| IPI00298497 | fibb_humanfibrinogen beta | P | GO: 0051179 | localization |
| | chain os = homo sapiens | | | |
| | gn = fgb pe = 1 sv = 2 | | | |
| IPI00298497 | fibb_humanfibrinogen beta | C | GO: 0031091 | platelet alpha granule |
| | chain os = homo sapiens | | | |
| | gn = fgb pe = 1 sv = 2 | | | |
| IPI00298497 | fibb_humanfibrinogen beta | C | GO: 0009897 | external side of plasma |
| | chain os = homo sapiens | | | membrane |
| | gn = fgb pe = 1 sv = 2 | | | |
| IPI00298497 | fibb_humanfibrinogen beta | P | GO: 0050794 | regulation of cellular process |
| | chain os = homo sapiens | | | |
| | gn = fgb pe = 1 sv = 2 | | | |
| IPI00298497 | fibb_humanfibrinogen beta | P | GO: 0006950 | response to stress |
| | chain os = homo sapiens | | | |
| | gn = fgb pe = 1 sv = 2 | | | |
| IPI00298497 | fibb_humanfibrinogen beta | F | GO: 0043499 | eukaryotic cell surface |
| | chain os = homo sapiens | | | binding |
| | gn = fgb pe = 1 sv = 2 | | | |
| IPI00298497 | fibb_humanfibrinogen beta | P | GO: 0032501 | multicellular organismal |
| | chain os = homo sapiens | | | process |
| | gn = fgb pe = 1 sv = 2 | | | |
| KH 37 | IPI00021841 | GammaRAAS | IPI00021841 | apoa1_humanapolipoprotein |
| | | | | a-i os = homo sapiens |
| | | | | gn = apoa1 pe = 1 sv = 1 |
| IPI00021841 | apoa1_humanapolipoprotein | P | GO: 0070508 | cholesterol import |
| | a-i os = homo sapiens | | | |
| | gn = apoa1 pe = 1 sv = 1 | | | |
| IPI00021841 | apoa1_humanapolipoprotein | C | GO: 0030139 | endocytic vesicle |
| | a-i os = homo sapiens | | | |
| | gn = apoa1 pe = 1 sv = 1 | | | |
| IPI00021841 | apoa1_humanapolipoprotein | P | GO: 0050728 | negative regulation of |
| | a-i os = homo sapiens | | | inflammatory response |
| | gn = apoa1 pe = 1 sv = 1 | | | |
| IPI00021841 | apoa1_humanapolipoprotein | P | GO: 0033344 | cholesterol efflux |
| | a-i os = homo sapiens | | | |
| | gn = apoa1 pe = 1 sv = 1 | | | |
| IPI00021841 | apoa1_humanapolipoprotein | P | GO: 0034115 | negative regulation of |
| | a-i os = homo sapiens | | | heterotypic cell-cell |
| | gn = apoa1 pe = 1 sv = 1 | | | adhesion |
| IPI00021841 | apoa1_humanapolipoprotein | P | GO: 0018206 | peptidyl-methionine |
| | a-i os = homo sapiens | | | modification |
| | gn = apoa1 pe = 1 sv = 1 | | | |
| IPI00021841 | apoa1_humanapolipoprotein | P | GO: 0042157 | lipoprotein metabolic |
| | a-i os = homo sapiens | | | process |
| | gn = apoa1 pe = 1 sv = 1 | | | |
| IPI00021841 | apoa1_humanapolipoprotein | P | GO: 0043691 | reverse cholesterol transport |
| | a-i os = homo sapiens | | | |
| | gn = apoa1 pe = 1 sv = 1 | | | |
| IPI00021841 | apoa1_humanapolipoprotein | F | GO: 0005543 | phospholipid binding |
| | a-i os = homo sapiens | | | |
| | gn = apoa1 pe = 1 sv = 1 | | | |
| IPI00021841 | apoa1_humanapolipoprotein | P | GO: 0002740 | negative regulation of |
| | a-i os = homo sapiens | | | cytokine secretion involved |
| | gn = apoa1 pe = 1 sv = 1 | | | in immune response |
| IPI00021841 | apoa1_humanapolipoprotein | P | GO: 0008203 | cholesterol metabolic |
| | a-i os = homo sapiens | | | process |
| | gn = apoa1 pe = 1 sv = 1 | | | |
| IPI00021841 | apoa1_humanapolipoprotein | P | GO: 0050713 | negative regulation of |
| | a-i os = homo sapiens | | | interleukin-1 beta secretion |
| | gn = apoa1 pe = 1 sv = 1 | | | |
| IPI00021841 | apoa1_humanapolipoprotein | P | GO: 0018158 | protein oxidation |
| | a-i os = homo sapiens | | | |
| | gn = apoa1 pe = 1 sv = 1 | | | |
| IPI00021841 | apoa1_humanapolipoprotein | P | GO: 0006656 | phosphatidylcholine |
| | a-i os = homo sapiens | | | biosynthetic process |
| | gn = apoa1 pe = 1 sv = 1 | | | |
| IPI00021841 | apoa1_humanapolipoprotein | F | GO: 0001540 | beta-amyloid binding |
| | a-i os = homo sapiens | | | |
| | gn = apoa1 pe = 1 sv = 1 | | | |
| IPI00021841 | apoa1_humanapolipoprotein | F | GO: 0060228 | phosphatidylcholine-sterol |
| | a-i os = homo sapiens | | | O-acyltransferase activator |
| | gn = apoa1 pe = 1 sv = 1 | | | activity |
| IPI00021841 | apoa1_humanapolipoprotein | P | GO: 0042632 | cholesterol homeostasis |
| | a-i os = homo sapiens | | | |
| | gn = apoa1 pe = 1 sv = 1 | | | |
| IPI00021841 | apoa1_humanapolipoprotein | F | GO: 0015485 | cholesterol binding |
| | a-i os = homo sapiens | | | |
| | gn = apoa1 pe = 1 sv = 1 | | | |
| IPI00021841 | apoa1_humanapolipoprotein | P | GO: 0042060 | wound healing |
| | a-i os = homo sapiens | | | |
| | gn = apoa1 pe = 1 sv = 1 | | | |
| IPI00021841 | apoa1_humanapolipoprotein | F | GO: 0034191 | apolipoprotein A-I receptor |
| | a-i os = homo sapiens | | | binding |
| | gn = apoa1 pe = 1 sv = 1 | | | |
| IPI00021841 | apoa1_humanapolipoprotein | F | GO: 0042802 | identical protein binding |
| | a-i os = homo sapiens | | | |
| | gn = apoa1 pe = 1 sv = 1 | | | |
| IPI00021841 | apoa1_humanapolipoprotein | P | GO: 0010903 | negative regulation of very- |
| | a-i os = homo sapiens | | | low-density lipoprotein |
| | gn = apoa1 pe = 1 sv = 1 | | | particle remodeling |
| IPI00021841 | apoa1_humanapolipoprotein | P | GO: 0010804 | negative regulation of tumor |
| | a-i os = homo sapiens | | | necrosis factor-mediated |
| | gn = apoa1 pe = 1 sv = 1 | | | signaling pathway |
| IPI00021841 | apoa1_humanapolipoprotein | P | GO: 0034380 | high-density lipoprotein |
| | a-i os = homo sapiens | | | particle assembly |
| | gn = apoa1 pe = 1 sv = 1 | | | |
| IPI00021841 | apoa1_humanapolipoprotein | P | GO: 0007186 | G-protein coupled receptor |
| | a-i os = homo sapiens | | | signaling pathway |
| | gn = apoa1 pe = 1 sv = 1 | | | |
| IPI00021841 | apoa1_humanapolipoprotein | P | GO: 0050821 | protein stabilization |
| | a-i os = homo sapiens | | | |
| | gn = apoa1 pe = 1 sv = 1 | | | |
| IPI00021841 | apoa1_humanapolipoprotein | C | GO: 0034361 | very-low-density lipoprotein |
| | a-i os = homo sapiens | | | particle |
| | gn = apoa1 pe = 1 sv = 1 | | | |
| IPI00021841 | apoa1_humanapolipoprotein | P | GO: 0032488 | Cdc42 protein signal |
| | a-i os = homo sapiens | | | transduction |
| | gn = apoa1 pe = 1 sv = 1 | | | |
| IPI00021841 | apoa1_humanapolipoprotein | P | GO: 0060354 | negative regulation of cell |
| | a-i os = homo sapiens | | | adhesion molecule |
| | gn = apoa1 pe = 1 sv = 1 | | | production |
| IPI00021841 | apoa1_humanapolipoprotein | P | GO: 0055091 | phospholipid homeostasis |
| | a-i os = homo sapiens | | | |
| | gn = apoa1 pe = 1 sv = 1 | | | |
| IPI00021841 | apoa1_humanapolipoprotein | P | GO: 0010873 | positive regulation of |
| | a-i os = homo sapiens | | | cholesterol esterification |
| | gn = apoa1 pe = 1 sv = 1 | | | |
| IPI00021841 | apoa1_humanapolipoprotein | F | GO: 0017127 | cholesterol transporter |
| | a-i os = homo sapiens | | | activity |
| | gn = apoa1 pe = 1 sv = 1 | | | |
| IPI00021841 | apoa1_humanapolipoprotein | F | GO: 0019899 | enzyme binding |
| | a-i os = homo sapiens | | | |
| | gn = apoa1 pe = 1 sv = 1 | | | |
| IPI00021841 | apoa1_humanapolipoprotein | F | GO: 0070653 | high-density lipoprotein |
| | a-i os = homo sapiens | | | particle receptor binding |
| | gn = apoa1 pe = 1 sv = 1 | | | |
| IPI00021841 | apoa1_humanapolipoprotein | P | GO: 0070328 | triglyceride homeostasis |
| | a-i os = homo sapiens | | | |
| | gn = apoa1 pe = 1 sv = 1 | | | |
| IPI00021841 | apoa1_humanapolipoprotein | C | GO: 0034366 | spherical high-density |
| | a-i os = homo sapiens | | | lipoprotein particle |
| | gn = apoa1 pe = 1 sv = 1 | | | |
| IPI00021841 | apoa1_humanapolipoprotein | P | GO: 0033700 | phospholipid efflux |
| | a-i os = homo sapiens | | | |
| | gn = apoa1 pe = 1 sv = 1 | | | |
| IPI00021841 | apoa1_humanapolipoprotein | P | GO: 0051345 | positive regulation of |
| | a-i os = homo sapiens | | | hydrolase activity |
| | gn = apoa1 pe = 1 sv = 1 | | | |
| KH 38 | IPI00783987 | AFCC | IPI00783987 | co3_humancomplement c3 |
| | | | | os = homo sapiens gn = c3 |
| | | | | pe = 1 sv = 2 |
| IPI00783987 | co3_humancomplement c3 | C | GO: 0044464 | cell part |
| | os = homo sapiens gn = c3 | | | |
| | pe = 1 sv = 2 | | | |
| IPI00783987 | co3_humancomplement c3 | P | GO: 0010575 | positive regulation vascular |
| | os = homo sapiens gn = c3 | | | endothelial growth factor |
| | pe = 1 sv = 2 | | | production |
| IPI00783987 | co3_humancomplement c3 | P | GO: 0030449 | regulation of complement |
| | os = homo sapiens gn = c3 | | | activation |
| | pe = 1 sv = 2 | | | |
| IPI00783987 | co3_humancomplement c3 | P | GO: 0007165 | signal transduction |
| | os = homo sapiens gn = c3 | | | |
| | pe = 1 sv = 2 | | | |
| IPI00783987 | co3_humancomplement c3 | P | GO: 0045087 | innate immune response |
| | os = homo sapiens gn = c3 | | | |
| | pe = 1 sv = 2 | | | |
| IPI00783987 | co3_humancomplement c3 | F | GO: 0005515 | protein binding |
| | os = homo sapiens gn = c3 | | | |
| | pe = 1 sv = 2 | | | |
| IPI00783987 | co3_humancomplement c3 | C | GO: 0016020 | membrane |
| | os = homo sapiens gn = c3 | | | |
| | pe = 1 sv = 2 | | | |
| KH 39 | IPI00878282 | AFCC | IPI00878282 | albu_humanserum albumin |
| | | | | os = homo sapiens gn = alb |
| | | | | pe = 1 sv = 2 |
| IPI00878282 | albu_humanserum albumin | P | GO: 0008202 | steroid metabolic process |
| | os = homo sapiens gn = alb | | | |
| | pe = 1 sv = 2 | | | |
| IPI00878282 | albu_humanserum albumin | F | GO: 0051087 | chaperone binding |
| | os = homo sapiens gn = alb | | | |
| | pe = 1 sv = 2 | | | |
| IPI00878282 | albu_humanserum albumin | C | GO: 0044446 | intracellular organelle part |
| | os = homo sapiens gn = alb | | | |
| | pe = 1 sv = 2 | | | |
| IPI00878282 | albu_humanserum albumin | F | GO: 0015643 | toxin binding |
| | os = homo sapiens gn = alb | | | |
| | pe = 1 sv = 2 | | | |
| IPI00878282 | albu_humanserum albumin | P | GO: 0044260 | cellular macromolecule |
| | os = homo sapiens gn = alb | | | metabolic process |
| | pe = 1 sv = 2 | | | |
| IPI00878282 | albu_humanserum albumin | C | GO: 0005615 | extracellular space |
| | os = homo sapiens gn = alb | | | |
| | pe = 1 sv = 2 | | | |
| IPI00878282 | albu_humanserum albumin | P | GO: 0051659 | maintenance of |
| | os = homo sapiens gn = alb | | | mitochondrion location |
| | pe = 1 sv = 2 | | | |
| IPI00878282 | albu_humanserum albumin | F | GO: 0008144 | drug binding |
| | os = homo sapiens gn = alb | | | |
| | pe = 1 sv = 2 | | | |
| IPI00878282 | albu_humanserum albumin | C | GO: 0043231 | intracellular membrane- |
| | os = homo sapiens gn = alb | | | bounded organelle |
| | pe = 1 sv = 2 | | | |
| IPI00878282 | albu_humanserum albumin | P | GO: 0044281 | small molecule metabolic |
| | os = homo sapiens gn = alb | | | process |
| | pe = 1 sv = 2 | | | |
| IPI00878282 | albu_humanserum albumin | F | GO: 0005504 | fatty acid binding |
| | os = homo sapiens gn = alb | | | |
| | pe = 1 sv = 2 | | | |
| IPI00878282 | albu_humanserum albumin | P | GO: 0042221 | response to chemical |
| | os = homo sapiens gn = alb | | | stimulus |
| | pe = 1 sv = 2 | | | |
| IPI00878282 | albu_humanserum albumin | F | GO: 0003677 | DNA binding |
| | os = homo sapiens gn = alb | | | |
| | pe = 1 sv = 2 | | | |
| IPI00878282 | albu_humanserum albumin | P | GO: 0009267 | cellular response to |
| | os = homo sapiens gn = alb | | | starvation |
| | pe = 1 sv = 2 | | | |
| IPI00878282 | albu_humanserum albumin | F | GO: 0030170 | pyridoxal phosphate binding |
| | os = homo sapiens gn = alb | | | |
| | pe = 1 sv = 2 | | | |
| IPI00878282 | albu_humanserum albumin | P | GO: 0006810 | transport |
| | os = homo sapiens gn = alb | | | |
| | pe = 1 sv = 2 | | | |
| IPI00878282 | albu_humanserum albumin | F | GO: 0019825 | oxygen binding |
| | os = homo sapiens gn = alb | | | |
| | pe = 1 sv = 2 | | | |
| IPI00878282 | albu_humanserum albumin | P | GO: 0050878 | regulation of body fluid |
| | os = homo sapiens gn = alb | | | levels |
| | pe = 1 sv = 2 | | | |
| IPI00878282 | albu_humanserum albumin | P | GO: 0043066 | negative regulation of |
| | os = homo sapiens gn = alb | | | apoptotic process |
| | pe = 1 sv = 2 | | | |
| IPI00878282 | albu_humanserum albumin | C | GO: 0044444 | cytoplasmic part |
| | os = homo sapiens gn = alb | | | |
| | pe = 1 sv = 2 | | | |
| IPI00878282 | albu_humanserum albumin | P | GO: 0009611 | response to wounding |
| | os = homo sapiens gn = alb | | | |
| | pe = 1 sv = 2 | | | |
| IPI00878282 | albu_humanserum albumin | P | GO: 0019836 | hemolysis by symbiont of |
| | os = homo sapiens gn = alb | | | host erythrocytes |
| | pe = 1 sv = 2 | | | |
| IPI00878282 | albu_humanserum albumin | P | GO: 0006955 | immune response |
| | os = homo sapiens gn = alb | | | |
| | pe = 1 sv = 2 | | | |
| IPI00878282 | albu_humanserum albumin | C | GO: 0019814 | immunoglobulin complex |
| | os = homo sapiens gn = alb | | | |
| | pe = 1 sv = 2 | | | |
| IPI00784842 | ighg1_humanig gamma-1 | P | GO: 0050776 | regulation of immune |
| | chain c region os = homo | | | response |
| | sapiens gn = ighg1 pe = 1 sv = 1 | | | |
| IPI00784842 | ighg1_humanig gamma-1 | F | GO: 0005515 | protein binding |
| | chain c region os = homo | | | |
| | sapiens gn = ighg1 pe = 1 sv = 1 | | | |
| KH 40 | IPI00784842 | AFCC | IPI00784842 | ighg1_humanig gamma-1 |
| | | | | chain c region os = homo |
| | | | | sapiens gn = ighg1 pe = 1 sv = 1 |
| IPI00784842 | ighg1_humanig gamma-1 | P | GO: 0050776 | regulation of immune |
| | chain c region os = homo | | | response |
| | sapiens gn = ighg1 pe = 1 sv = 1 | | | |
| IPI00784842 | ighg1_humanig gamma-1 | F | GO: 0005515 | protein binding |
| | chain c region os = homo | | | |
| | sapiens gn = ighg1 pe = 1 sv = 1 | | | |
| KH 41 | IPI00022434 | Fraction III- | same as | |
| | | II | KH 28 | |
| KH 42 | IPI00298497 | Fraction III | same as | |
| | | | KH 36 | |
| KH 43 | IPI00965713 | Fraction III | IPI00965713 | fibb_humanfibrinogen beta |
| | | | | chain os = homo sapiens |
| | | | | gn = fgb pe = 1 sv = 2 |
| IPI00965713 | fibb_humanfibrinogen beta | P | GO: 0042221 | response to chemical |
| | chain os = homo sapiens | | | stimulus |
| | gn = fgb pe = 1 sv = 2 | | | |
| IPI00965713 | fibb_humanfibrinogen beta | F | GO: 0005515 | protein binding |
| | chain os = homo sapiens | | | |
| | gn = fgb pe = 1 sv = 2 | | | |
| IPI00965713 | fibb_humanfibrinogen beta | C | GO: 0005615 | extracellular space |
| | chain os = homo sapiens | | | |
| | gn = fgb pe = 1 sv = 2 | | | |
| IPI00965713 | fibb_humanfibrinogen beta | P | GO: 0051179 | localization |
| | chain os = homo sapiens | | | |
| | gn = fgb pe = 1 sv = 2 | | | |
| IPI00965713 | fibb_humanfibrinogen beta | C | GO: 0031091 | platelet alpha granule |
| | chain os = homo sapiens | | | |
| | gn = fgb pe = 1 sv = 2 | | | |
| IPI00965713 | fibb_humanfibrinogen beta | C | GO: 0044425 | membrane part |
| | chain os = homo sapiens | | | |
| | gn = fgb pe = 1 sv = 2 | | | |
| IPI00965713 | fibb_humanfibrinogen beta | P | GO: 0050794 | regulation of cellular process |
| | chain os = homo sapiens | | | |
| | gn = fgb pe = 1 sv = 2 | | | |
| IPI00965713 | fibb_humanfibrinogen beta | P | GO: 0006950 | response to stress |
| | chain os = homo sapiens | | | |
| | gn = fgb pe = 1 sv = 2 | | | |
| IPI00965713 | fibb_humanfibrinogen beta | C | GO: 0005886 | plasma membrane |
| | chain os = homo sapiens | | | |
| | gn = fgb pe = 1 sv = 2 | | | |
| IPI00965713 | fibb_humanfibrinogen beta | P | GO: 0032501 | multicellular organismal |
| | chain os = homo sapiens | | | process |
| | gn = fgb pe = 1 sv = 2 | | | |
| KH 44 | IPI00645363 | FibringluRAAS ® | IPI00645363 | ighg1_humanig gamma-1 |
| | | Human | | chain c region os = homo |
| | | Thrombin | | sapiens gn = ighg1 pe = 1 sv = 1 |
| IPI00645363 | ighg1_humanig gamma-1 | P | GO: 0050776 | regulation of immune |
| | chain c region os = homo | | | response |
| | sapiens gn = ighg1 pe = 1 sv = 1 | | | |
| IPI00645363 | ighg1_humanig gamma-1 | F | GO: 0005515 | protein binding |
| | chain c region os = homo | | | |
| | sapiens gn = ighg1 pe = 1 sv = 1 | | | |
| KH 45 | IPI00219713 | FibringluRAAS ® | same as | |
| | | Human | KH 30 | |
| | | Thrombin | | |
| IPI00219713 | fibg_humanfibrinogen | P | GO: 0009987 | cellular process |
| | gamma chain os = homo | | | |
| | sapiens gn = fgg pe = 1 sv = 3 | | | |
| IPI00219713 | fibg_humanfibrinogen | C | GO: 0009897 | external side of plasma |
| | gamma chain os = homo | | | membrane |
| | sapiens gn = fgg pe = 1 sv = 3 | | | |
| IPI00219713 | fibg_humanfibrinogen | F | GO: 0043499 | eukaryotic cell surface |
| | gamma chain os = homo | | | binding |
| | sapiens gn = fgg pe = 1 sv = 3 | | | |
| IPI00219713 | fibg_humanfibrinogen | C | GO: 0005615 | extracellular space |
| | gamma chain os = homo | | | |
| | sapiens gn = fgg pe = 1 sv = 3 | | | |
| IPI00219713 | fibg_humanfibrinogen | C | GO: 0031091 | platelet alpha granule |
| | gamma chain os = homo | | | |
| | sapiens gn = fgg pe = 1 sv = 3 | | | |
| IPI00219713 | fibg_humanfibrinogen | P | GO: 0032501 | multicellular organismal |
| | gamma chain os = homo | | | process |
| | sapiens gn = fgg pe = 1 sv = 3 | | | |
| IPI00219713 | fibg_humanfibrinogen | P | GO: 0065007 | biological regulation |
| | gamma chain os = homo | | | |
| | sapiens gn = fgg pe = 1 sv = 3 | | | |
| IPI00219713 | fibg_humanfibrinogen | P | GO: 0051592 | response to calcium ion |
| | gamma chain os = homo | | | |
| | sapiens gn = fgg pe = 1 sv = 3 | | | |
| KH 46 | IPI00022371 | FibringluRAAS ® | IPI00022371 | hrg_humanhistidine-rich |
| | | Human | | glycoprotein os = homo |
| | | Thrombin | | sapiens gn = hrg pe = 1 sv = 1 |
| IPI00022371 | hrg_humanhistidine-rich | P | GO: 0043065 | positive regulation of |
| | glycoprotein os = homo | | | apoptotic process |
| | sapiens gn = hrg pe = 1 sv = 1 | | | |
| IPI00022371 | hrg_humanhistidine-rich | P | GO: 0010468 | regulation of gene |
| | glycoprotein os = homo | | | expression |
| | sapiens gn = hrg pe = 1 sv = 1 | | | |
| IPI00022371 | hrg_humanhistidine-rich | P | GO: 0032956 | regulation of actin |
| | glycoprotein os = homo | | | cytoskeleton organization |
| | sapiens gn = hrg pe = 1 sv = 1 | | | |
| IPI00022371 | hrg_humanhistidine-rich | P | GO: 0016525 | negative regulation of |
| | glycoprotein os = homo | | | angiogenesis |
| | sapiens gn = hrg pe = 1 sv = 1 | | | |
| IPI00022371 | hrg_humanhistidine-rich | P | GO: 2000504 | positive regulation of blood |
| | glycoprotein os = homo | | | vessel remodeling |
| | sapiens gn = hrg pe = 1 sv = 1 | | | |
| IPI00022371 | hrg_humanhistidine-rich | P | GO: 0043254 | regulation of protein |
| | glycoprotein os = homo | | | complex assembly |
| | sapiens gn = hrg pe = 1 sv = 1 | | | |
| IPI00022371 | hrg_humanhistidine-rich | P | GO: 0002839 | positive regulation of |
| | glycoprotein os = homo | | | immune response to tumor |
| | sapiens gn = hrg pe = 1 sv = 1 | | | cell |
| IPI00022371 | hrg_humanhistidine-rich | F | GO: 0008201 | heparin binding |
| | glycoprotein os = homo | | | |
| | sapiens gn = hrg pe = 1 sv = 1 | | | |
| IPI00022371 | hrg_humanhistidine-rich | P | GO: 0010593 | negative regulation of |
| | glycoprotein os = homo | | | lamellipodium assembly |
| | sapiens gn = hrg pe = 1 sv = 1 | | | |
| IPI00022371 | hrg_humanhistidine-rich | P | GO: 0050832 | defense response to fungus |
| | glycoprotein os = homo | | | |
| | sapiens gn = hrg pe = 1 sv = 1 | | | |
| IPI00022371 | hrg_humanhistidine-rich | F | GO: 0020037 | heme binding |
| | glycoprotein os = homo | | | |
| | sapiens gn = hrg pe = 1 sv = 1 | | | |
| IPI00022371 | hrg_humanhistidine-rich | F | GO: 0019865 | immunoglobulin binding |
| | glycoprotein os = homo | | | |
| | sapiens gn = hrg pe = 1 sv = 1 | | | |
| IPI00022371 | hrg_humanhistidine-rich | P | GO: 0030168 | platelet activation |
| | glycoprotein os = homo | | | |
| | sapiens gn = hrg pe = 1 sv = 1 | | | |
| IPI00022371 | hrg_humanhistidine-rich | F | GO: 0043395 | heparan sulfate proteoglycan |
| | glycoprotein os = homo | | | binding |
| | sapiens gn = hrg pe = 1 sv = 1 | | | |
| IPI00022371 | hrg_humanhistidine-rich | P | GO: 1900747 | negative regulation of |
| | glycoprotein os = homo | | | vascular endothelial growth |
| | sapiens gn = hrg pe = 1 sv = 1 | | | factor signaling pathway |
| IPI00022371 | hrg_humanhistidine-rich | F | GO: 0008270 | zinc ion binding |
| | glycoprotein os = homo | | | |
| | sapiens gn = hrg pe = 1 sv = 1 | | | |
| IPI00022371 | hrg_humanhistidine-rich | F | GO: 0043498 | cell surface binding |
| | glycoprotein os = homo | | | |
| | sapiens gn = hrg pe = 1 sv = 1 | | | |
| IPI00022371 | hrg_humanhistidine-rich | P | GO: 2001027 | negative regulation of |
| | glycoprotein os = homo | | | endothelial cell chemotaxis |
| | sapiens gn = hrg pe = 1 sv = 1 | | | |
| IPI00022371 | hrg_humanhistidine-rich | P | GO: 0008285 | negative regulation of cell |
| | glycoprotein os = homo | | | proliferation |
| | sapiens gn = hrg pe = 1 sv = 1 | | | |
| IPI00022371 | hrg_humanhistidine-rich | P | GO: 0051894 | positive regulation of focal |
| | glycoprotein os = homo | | | adhesion assembly |
| | sapiens gn = hrg pe = 1 sv = 1 | | | |
| IPI00022371 | hrg_humanhistidine-rich | P | GO: 0030193 | regulation of blood |
| | glycoprotein os = homo | | | coagulation |
| | sapiens gn = hrg pe = 1 sv = 1 | | | |
| IPI00022371 | hrg_humanhistidine-rich | P | GO: 0050730 | regulation of peptidyl- |
| | glycoprotein os = homo | | | tyrosine phosphorylation |
| | sapiens gn = hrg pe = 1 sv = 1 | | | |
| IPI00022371 | hrg_humanhistidine-rich | P | GO: 0033629 | negative regulation of cell |
| | glycoprotein os = homo | | | adhesion mediated by |
| | sapiens gn = hrg pe = 1 sv = 1 | | | integrin |
| IPI00022371 | hrg_humanhistidine-rich | P | GO: 0030308 | negative regulation of cell |
| | glycoprotein os = homo | | | growth |
| | sapiens gn = hrg pe = 1 sv = 1 | | | |
| IPI00022371 | hrg_humanhistidine-rich | C | GO: 0005576 | extracellular region |
| | glycoprotein os = homo | | | |
| | sapiens gn = hrg pe = 1 sv = 1 | | | |
| IPI00022371 | hrg_humanhistidine-rich | C | GO: 0019814 | immunoglobulin complex |
| | glycoprotein os = homo | | | |
| | sapiens gn = hrg pe = 1 sv = 1 | | | |
| KH 47 | IPI00022371 | FibringluRAAS ® | same as | |
| | | Human | KH 46 | |
| | | Thrombin | | |
| KH 48 | IPI00022463 | AFOD | IPI00022463 | trfe_humanserotransferrin |
| | | | | os = homo sapiens gn = tf pe = 1 |
| | | | | sv = 3 |
| IPI00022463 | trfe_humanserotransferrin | P | GO: 0009987 | cellular process |
| | os = homo sapiens gn = tf pe = 1 | | | |
| | sv = 3 | | | |
| IPI00022463 | trfe_humanserotransferrin | P | GO: 0065008 | regulation of biological |
| | os = homo sapiens gn = tf pe = 1 | | | quality |
| | sv = 3 | | | |
| IPI00022463 | trfe_humanserotransferrin | P | GO: 0006810 | transport |
| | os = homo sapiens gn = tf pe = 1 | | | |
| | sv = 3 | | | |
| IPI00022463 | trfe_humanserotransferrin | C | GO: 0009925 | basal plasma membrane |
| | os = homo sapiens gn = tf pe = 1 | | | |
| | sv = 3 | | | |
| IPI00022463 | trfe_humanserotransferrin | C | GO: 0005739 | mitochondrion |
| | os = homo sapiens gn = tf pe = 1 | | | |
| | sv = 3 | | | |
| IPI00022463 | trfe_humanserotransferrin | C | GO: 0030139 | endocytic vesicle |
| | os = homo sapiens gn = tf pe = 1 | | | |
| | sv = 3 | | | |
| IPI00022463 | trfe_humanserotransferrin | C | GO: 0005905 | coated pit |
| | os = homo sapiens gn = tf pe = 1 | | | |
| | sv = 3 | | | |
| IPI00022463 | trfe_humanserotransferrin | C | GO: 0005770 | late endosome |
| | os = homo sapiens gn = tf pe = 1 | | | |
| | sv = 3 | | | |
| IPI00022463 | trfe_humanserotransferrin | C | GO: 0005769 | early endosome |
| | os = homo sapiens gn = tf pe = 1 | | | |
| | sv = 3 | | | |
| IPI00022463 | trfe_humanserotransferrin | C | GO: 0055037 | recycling endosome |
| | os = homo sapiens gn = tf pe = 1 | | | |
| | sv = 3 | | | |
| IPI00022463 | trfe_humanserotransferrin | F | GO: 0005515 | protein binding |
| | os = homo sapiens gn = tf pe = 1 | | | |
| | sv = 3 | | | |
| IPI00022463 | trfe_humanserotransferrin | C | GO: 0048471 | perinuclear region of |
| | os = homo sapiens gn = tf pe = 1 | | | cytoplasm |
| | sv = 3 | | | |
| IPI00022463 | trfe_humanserotransferrin | C | GO: 0016324 | apical plasma membrane |
| | os = homo sapiens gn = tf pe = 1 | | | |
| | sv = 3 | | | |
| IPI00022463 | trfe_humanserotransferrin | P | GO: 0006950 | response to stress |
| | os = homo sapiens gn = tf pe = 1 | | | |
| | sv = 3 | | | |
| KH 49 | IPI00023006 | AFOD | IPI00023006 | alpha cardiac muscle 1 |
| | | | | os = homo sapiens gn = actc1 |
| | | | | pe = 1 sv = 1 |
| IPI00023006 | alpha cardiac muscle 1 | C | GO: 0005865 | striated muscle thin filament |
| | os = homo sapiens gn = actc1 | | | |
| | pe = 1 sv = 1 | | | |
| IPI00023006 | alpha cardiac muscle 1 | F | GO: 0017022 | myosin binding |
| | os = homo sapiens gn = actc1 | | | |
| | pe = 1 sv = 1 | | | |
| IPI00023006 | alpha cardiac muscle 1 | P | GO: 0030240 | skeletal muscle thin filament |
| | os = homo sapiens gn = actc1 | | | assembly |
| | pe = 1 sv = 1 | | | |
| IPI00023006 | alpha cardiac muscle 1 | P | GO: 0006200 | ATP catabolic process |
| | os = homo sapiens gn = actc1 | | | |
| | pe = 1 sv = 1 | | | |
| IPI00023006 | alpha cardiac muscle 1 | P | GO: 0072144 | glomerular mesangial cell |
| | os = homo sapiens gn = actc1 | | | development |
| | pe = 1 sv = 1 | | | |
| IPI00023006 | alpha cardiac muscle 1 | P | GO: 0006936 | muscle contraction |
| | os = homo sapiens gn = actc1 | | | |
| | pe = 1 sv = 1 | | | |
| IPI00023006 | alpha cardiac muscle 1 | P | GO: 0033275 | actin-myosin filament |
| | os = homo sapiens gn = actc1 | | | sliding |
| | pe = 1 sv = 1 | | | |
| IPI00023006 | alpha cardiac muscle 1 | C | GO: 0042643 | actomyosin, actin part |
| | os = homo sapiens gn = actc1 | | | |
| | pe = 1 sv = 1 | | | |
| IPI00023006 | alpha cardiac muscle 1 | P | GO: 0042221 | response to chemical |
| | os = homo sapiens gn = actc1 | | | stimulus |
| | pe = 1 sv = 1 | | | |
| IPI00023006 | alpha cardiac muscle 1 | F | GO: 0005524 | ATP binding |
| | os = homo sapiens gn = actc1 | | | |
| | pe = 1 sv = 1 | | | |
| IPI00023006 | alpha cardiac muscle 1 | C | GO: 0001725 | stress fiber |
| | os = homo sapiens gn = actc1 | | | |
| | pe = 1 sv = 1 | | | |
| IPI00023006 | alpha cardiac muscle 1 | F | GO: 0016887 | ATPase activity |
| | os = homo sapiens gn = actc1 | | | |
| | pe = 1 sv = 1 | | | |
| IPI00023006 | alpha cardiac muscle 1 | P | GO: 0065008 | regulation of biological |
| | os = homo sapiens gn = actc1 | | | quality |
| | pe = 1 sv = 1 | | | |
| IPI00023006 | alpha cardiac muscle 1 | C | GO: 0044451 | nucleoplasm part |
| | os = homo sapiens gn = actc1 | | | |
| | pe = 1 sv = 1 | | | |
| IPI00023006 | alpha cardiac muscle 1 | P | GO: 0009615 | response to virus |
| | os = homo sapiens gn = actc1 | | | |
| | pe = 1 sv = 1 | | | |
| IPI00023006 | alpha cardiac muscle 1 | P | GO: 0060047 | heart contraction |
| | os = homo sapiens gn = actc1 | | | |
| | pe = 1 sv = 1 | | | |
| IPI00023006 | alpha cardiac muscle 1 | F | GO: 0019899 | enzyme binding |
| | os = homo sapiens gn = actc1 | | | |
| | pe = 1 sv = 1 | | | |
| IPI00023006 | alpha cardiac muscle 1 | C | GO: 0016459 | myosin complex |
| | os = homo sapiens gn = actc1 | | | |
| | pe = 1 sv = 1 | | | |
| KH 50 | IPI00021841 | AFOD | same as | |
| | | | KH 37 | |
| KH 51 | IPI00023006 | AlbuRAAS | same as | |
| | | | KH 49 | |
| KH 52 | IPI00930226 | FibringluRAAS ® | IPI00930226 | cytoplasmic 2 os = homo |
| | | High | | sapiens gn = actg1 pe = 1 sv = 1 |
| | | Concentrate | | |
| | | Human | | |
| | | Fibrinogen | | |
| IPI00930226 | cytoplasmic 2 os = homo | P | GO: 0009888 | tissue development |
| | sapiens gn = actg1 pe = 1 sv = 1 | | | |
| IPI00930226 | cytoplasmic 2 os = homo | P | GO: 0030048 | actin filament-based |
| | sapiens gn = actg1 pe = 1 sv = 1 | | | movement |
| IPI00930226 | cytoplasmic 2 os = homo | P | GO: 0003012 | muscle system process |
| | sapiens gn = actg1 pe = 1 sv = 1 | | | |
| IPI00930226 | cytoplasmic 2 os = homo | C | GO: 0030017 | sarcomere |
| | sapiens gn = actg1 pe = 1 sv = 1 | | | |
| IPI00930226 | cytoplasmic 2 os = homo | P | GO: 0030239 | myofibril assembly |
| | sapiens gn = actg1 pe = 1 sv = 1 | | | |
| IPI00930226 | cytoplasmic 2 os = homo | P | GO: 0044238 | primary metabolic process |
| | sapiens gn = actg1 pe = 1 sv = 1 | | | |
| IPI00930226 | cytoplasmic 2 os = homo | C | GO: 0005884 | actin filament |
| | sapiens gn = actg1 pe = 1 sv = 1 | | | |
| IPI00930226 | cytoplasmic 2 os = homo | P | GO: 0072358 | cardiovascular system |
| | sapiens gn = actg1 pe = 1 sv = 1 | | | development |
| IPI00930226 | cytoplasmic 2 os = homo | P | GO: 0044237 | cellular metabolic process |
| | sapiens gn = actg1 pe = 1 sv = 1 | | | |
| IPI00930226 | cytoplasmic 2 os = homo | P | GO: 0048513 | organ development |
| | sapiens gn = actg1 pe = 1 sv = 1 | | | |
| IPI00930226 | cytoplasmic 2 os = homo | P | GO: 0042221 | response to chemical |
| | sapiens gn = actg1 pe = 1 sv = 1 | | | stimulus |
| IPI00930226 | cytoplasmic 2 os = homo | F | GO: 0008092 | cytoskeletal protein binding |
| | sapiens gn = actg1 pe = 1 sv = 1 | | | |
| IPI00930226 | cytoplasmic 2 os = homo | P | GO: 0065008 | regulation of biological |
| | sapiens gn = actg1 pe = 1 sv = 1 | | | quality |
| IPI00930226 | cytoplasmic 2 os = homo | C | GO: 0044451 | nucleoplasm part |
| | sapiens gn = actg1 pe = 1 sv = 1 | | | |
| IPI00930226 | cytoplasmic 2 os = homo | P | GO: 0008015 | blood circulation |
| | sapiens gn = actg1 pe = 1 sv = 1 | | | |
| IPI00930226 | cytoplasmic 2 os = homo | F | GO: 0019899 | enzyme binding |
| | sapiens gn = actg1 pe = 1 sv = 1 | | | |
| KH 53 | 194373497 | AFCC | gi\|194373497 | thrb_humanprothrombin |
| | | (Fraction IV) | | os = homo sapiens gn = f2 |
| | | | | pe = 1 sv = 2 |
| gi\|194373497 | thrb_humanprothrombin | C | GO: 0044446 | intracellular organelle part |
| | os = homo sapiens gn = f2 | | | |
| | pe = 1 sv = 2 | | | |
| gi\|194373497 | thrb_humanprothrombin | P | GO: 0048712 | negative regulation of |
| | os = homo sapiens gn = f2 | | | astrocyte differentiation |
| | pe = 1 sv = 2 | | | |
| gi\|194373497 | thrb_humanprothrombin | C | GO: 0043233 | organelle lumen |
| | os = homo sapiens gn = f2 | | | |
| | pe = 1 sv = 2 | | | |
| gi\|194373497 | thrb_humanprothrombin | P | GO: 0030194 | positive regulation of blood |
| | os = homo sapiens gn = f2 | | | coagulation |
| | pe = 1 sv = 2 | | | |
| gi\|194373497 | thrb_humanprothrombin | F | GO: 0005102 | receptor binding |
| | os = homo sapiens gn = f2 | | | |
| | pe = 1 sv = 2 | | | |
| gi\|194373497 | thrb_humanprothrombin | P | GO: 2000379 | positive regulation of |
| | os = homo sapiens gn = f2 | | | reactive oxygen species |
| | pe = 1 sv = 2 | | | metabolic process |
| gi\|194373497 | thrb_humanprothrombin | P | GO: 0045861 | negative regulation of |
| | os = homo sapiens gn = f2 | | | proteolysis |
| | pe = 1 sv = 2 | | | |
| gi\|194373497 | thrb_humanprothrombin | C | GO: 0005615 | extracellular space |
| | os = homo sapiens gn = f2 | | | |
| | pe = 1 sv = 2 | | | |
| gi\|194373497 | thrb_humanprothrombin | P | GO: 0030168 | platelet activation |
| | os = homo sapiens gn = f2 | | | |
| | pe = 1 sv = 2 | | | |
| gi\|194373497 | thrb_humanprothrombin | P | GO: 1900738 | positive regulation of |
| | os = homo sapiens gn = f2 | | | phospholipase C-activating |
| | pe = 1 sv = 2 | | | G-protein coupled receptor |
| | | | | signaling pathway |
| gi\|194373497 | thrb_humanprothrombin | P | GO: 0016477 | cell migration |
| | os = homo sapiens gn = f2 | | | |
| | pe = 1 sv = 2 | | | |
| gi\|194373497 | thrb_humanprothrombin | C | GO: 0043231 | intracellular membrane- |
| | os = homo sapiens gn = f2 | | | bounded organelle |
| | pe = 1 sv = 2 | | | |
| gi\|194373497 | thrb_humanprothrombin | P | GO: 0001934 | positive regulation of protein |
| | os = homo sapiens gn = f2 | | | phosphorylation |
| | pe = 1 sv = 2 | | | |
| gi\|194373497 | thrb_humanprothrombin | C | GO: 0005886 | plasma membrane |
| | os = homo sapiens gn = f2 | | | |
| | pe = 1 sv = 2 | | | |
| gi\|194373497 | thrb_humanprothrombin | F | GO: 0070053 | thrombospondin receptor |
| | os = homo sapiens gn = f2 | | | activity |
| | pe = 1 sv = 2 | | | |
| gi\|194373497 | thrb_humanprothrombin | P | GO: 0051281 | positive regulation of release |
| | os = homo sapiens gn = f2 | | | of sequestered calcium ion |
| | pe = 1 sv = 2 | | | into cytosol |
| gi\|194373497 | thrb_humanprothrombin | F | GO: 0004252 | serine-type endopeptidase |
| | os = homo sapiens gn = f2 | | | activity |
| | pe = 1 sv = 2 | | | |
| gi\|194373497 | thrb_humanprothrombin | P | GO: 0042730 | fibrinolysis |
| | os = homo sapiens gn = f2 | | | |
| | pe = 1 sv = 2 | | | |
| gi\|194373497 | thrb_humanprothrombin | C | GO: 0044444 | cytoplasmic part |
| | os = homo sapiens gn = f2 | | | |
| | pe = 1 sv = 2 | | | |
| gi\|194373497 | thrb_humanprothrombin | P | GO: 0032967 | positive regulation of |
| | os = homo sapiens gn = f2 | | | collagen biosynthetic |
| | pe = 1 sv = 2 | | | process |
| KH 54 | 194380034 | Transferrin | gi\|194380034 | trfe_humanserotransferrin |
| | | | | os = homo sapiens gn = tf pe = 1 |
| | | | | sv = 3 |
| gi\|194380034 | trfe_humanserotransferrin | P | GO: 0009987 | cellular process |
| | os = homo sapiens gn = tf pe = 1 | | | |
| | sv = 3 | | | |
| gi\|194380034 | trfe_humanserotransferrin | P | GO: 0065008 | regulation of biological |
| | os = homo sapiens gn = tf pe = 1 | | | quality |
| | sv = 3 | | | |
| gi\|194380034 | trfe_humanserotransferrin | P | GO: 0006810 | transport |
| | os = homo sapiens gn = tf pe = 1 | | | |
| | sv = 3 | | | |
| gi\|194380034 | trfe_humanserotransferrin | C | GO: 0009925 | basal plasma membrane |
| | os = homo sapiens gn = tf pe = 1 | | | |
| | sv = 3 | | | |
| gi\|194380034 | trfe_humanserotransferrin | C | GO: 0005739 | mitochondrion |
| | os = homo sapiens gn = tf pe = 1 | | | |
| | sv = 3 | | | |
| gi\|194380034 | trfe_humanserotransferrin | C | GO: 0030139 | endocytic vesicle |
| | os = homo sapiens gn = tf pe = 1 | | | |
| | sv = 3 | | | |
| gi\|194380034 | trfe_humanserotransferrin | C | GO: 0005905 | coated pit |
| | os = homo sapiens gn = tf pe = 1 | | | |
| | sv = 3 | | | |
| gi\|194380034 | trfe_humanserotransferrin | C | GO: 0005770 | late endosome |
| | os = homo sapiens gn = tf pe = 1 | | | |
| | sv = 3 | | | |
| gi\|194380034 | trfe_humanserotransferrin | C | GO: 0005769 | early endosome |
| | os = homo sapiens gn = tf pe = 1 | | | |
| | sv = 3 | | | |
| gi\|194380034 | trfe_humanserotransferrin | C | GO: 0055037 | recycling endosome |
| | os = homo sapiens gn = tf pe = 1 | | | |
| | sv = 3 | | | |
| gi\|194380034 | trfe_humanserotransferrin | F | GO: 0005515 | protein binding |
| | os = homo sapiens gn = tf pe = 1 | | | |
| | sv = 3 | | | |
| gi\|194380034 | trfe_humanserotransferrin | C | GO: 0048471 | perinuclear region of |
| | os = homo sapiens gn = tf pe = 1 | | | cytoplasm |
| | sv = 3 | | | |
| gi\|194380034 | trfe_humanserotransferrin | C | GO: 0016324 | apical plasma membrane |
| | os = homo sapiens gn = tf pe = 1 | | | |
| | sv = 3 | | | |
| gi\|194380034 | trfe_humanserotransferrin | P | GO: 0006950 | response to stress |
| | os = homo sapiens gn = tf pe = 1 | | | |
| | sv = 3 | | | |
| KH 55 | 194380034 | Transferrin | same as | |
| | | | KH 54 | |

Protein sequence data as well as sequence identifiers and accession numbers for KH proteins 1-55 are found in the table below.

| KH | | | |
|---|---|---|---|
| Protein/SEQ | Sequence | | |
| ID NO | Identifier(s) | Protein Sequence | |
| 1 | gi: 21749960 | MDTYIESHFA GALAYRDLIK VLKWYVDRIT EAERQEHIQE VLKAQEYIFK YIVQSRRLFS | 60 |
| | BAC03696.1 | LATGGQNEEE FRCCIQELLM SVRFFLSQES KGSGALSQSQ AVFLSSFPAV YSELLKLFDV | 120 |
| | | REVANLVQDT LGSLPTILHV DDSLQAIKLQ CIGKTVESQL YTNPDSRYIL LPVVLHHLHI | 180 |
| | | HLQEQKDLIM CARILSNVFC LIKKNSSEKS VLEEIDVIVA SLLDILLRTI LEITSRPQPS | 240 |
| | | SSAMRFQFQD VTGEFVACLL SLLRQMTDRH YQQLLDSFNT KEELRDFLLQ IFTVFRILIR | 300 |
| | | PEMFPKDWTV MRLVANNVII TTVLYLSDAL RKNFLNENFD YKIWDSYFYL AVIFINQLCL | 360 |
| | | QLEMFTPSKK KKVLEKYGDM RVTMGCEIFS MWQNLGEHKL HFIPALIGPF LEVTLIPQPD | 420 |
| | | LRNVMIPIFH DMMDWEQRRS GNFKQVEAKL IDKLDSLMSE GEGDETYREL FNSIIPLFGP | 480 |
| | | YPSLLKKIER ETWRESGVSL IATVTRLMER LLDYRDCMKM GEVDGKKIGC TVSLLNFYKT | 540 |
| | | ELNKEEMYIR YIHKLYDLHL KAQNFTEAAY TLLLYDELLE WSDRPLREFL TYPMQTEWQR | 600 |
| | | KEHLHLTIIQ NFDRGKCWEN GIILCRKIAE QYESYYDYRN LSKMRMMEAS LYDKIMDQQR | 660 |
| | | LEPEFFRVGF YGKKFPFFLR NKEFVCRGHD YERLEAFQQR MLNEFPHAIA MQHANQPDET | 720 |
| | | IFQAEAQYLQ IYAVTPIPES QEVLQREGVP DNIKSFYKVN HIWKFRYDRP FHKGT | 775 |
| 2 | gi: 215415640 | DEPPQSPWDR VKDLATVYVD VLKDSGRDYV SQFEGSALGK QLNLKLLDNW DSVTSTFSKL | 60 |
| | CAT02162.1 | REQLGPVTQE FWDNLEKETE GLRQEMSKDL EEVKAKVQPY LDDFQKKWQE EMELYRQKVE | 120 |
| | | PLRAELQEGA RQKLHELQEK LSPLGEEMRD CARAHVDALR THLAPYSDEL RQRLAARLEA | 180 |
| | | LKENGGARLA EYHAKATEHL STLSEKAKPA LEDLRQGLLP VLESFKVSFL SALEEYTKKL | 240 |
| | | N | 241 |
| 3 | gi: 215415638 | DEPPQSPWDR VKDLATVYVD VLKDSGRDYV SQFEGSALGK QLNLKLLDNW DSVTSTFSKL | 60 |
| | CAT02161.1 | REQLGPVTQE FWDNLEKETE GLCQEMSKDL EEVKAKVQPY LDDFQKKWQE EMELYRQKVE | 120 |
| | | PLRAELQEGA RQKLHELQEK LSPLGEEMRD RARAHVDALR THLAPYSDEL RQRLAARLEA | 180 |
| | | LKENGGARLA EYHAKATEHL STLSEKAKPA LEDLRQGLLP VLESFKVSFL SALEEYTKKL | 240 |
| | | NTQ | 243 |
| 4 | gi: 40044478 | MGCKRASEVC GXAVEGLRDP LKPSEPSQGA AGKRKGTEYL MKQKLEFGGR GEELLLGVHL | 60 |
| | CAF01015.1 | RGAQKTGGGW RR | 72 |
| 5 | gi: 194383496 | TATDVFWAKY TACETARTPR DKLAACLEGN CAEGLGTNYR GHVNITRSGI ECQLWRSRYP | 120 |
| | BAG64719.1 | HKPEINSTTH PGADLQENFC RNPDSSTTGP WCYTTDPTVR RQECSIPVCG QDQVTVAMTP | 180 |
| | | GQQYQGRLAV TTHGLPCLAW ASAQAKALSK HQDFNSAVQL | 240 |
| | | VENFCRNPDG DEEGVWCYVA GKPGDFGYCD LNYCEEAVEE ETGDGLDEDS DRAIEGRTAT | 300 |
| | | SEYQTFFNPR TFGSGEADCG LRPLFEKKSL EDKTERELLE SYIDGRIVEG SDAEIGMSPW | 360 |
| | | QVMLFRKSPQ ELLCGASLIS DRWVLTAAHC LLYPPWDKNF TENDLLVRIG KHSRTRYERN | 420 |
| | | IEKISMLEKI YIHPRYNWRE NLDRDIALMK LKKPVAFSDY IHPVCLPDRE TAASLLQAGY | 480 |
| | | KGRVTGWGNL KETWTANVGK GQPSVLQVVN LPIVERPVCK DSTRIRITDN MFCAGYKPDE | 540 |
| | | GKRGDACEGD SGGPFVMKSP FNNRWYQMGI VSWGEGCDRD GKYGFYTHVF RLKKWIQKVI | 600 |
| | | DQFGE | 605 |
| 6 | gi: 28071026 | MQGTDEHVVC KVQHPNGNKE KNVPLPVIAE LPPKVSVFVP PRDGFFGNPR KSKLICQATG | 60 |
| | CAD61894.1 | FSPRQIQVSW LREGKQVGSG VTTDQVQAEA KESGPTTYKV TSTLTIKESD WLSQSMFTCR | 120 |
| | | VDHRGLTFQQ NASSMCGPDQ DTAIRVFAIP PSFASIFLTK STKLTCLVTD LTTYDSVTIS | 180 |
| | | WTRQNGEAVK THTNISESHP NATFSAVGEA SICEDDWNSG ERFTCTVTHT DLPSPLKQTI | 240 |
| | | SRPKGVALHR PDVYLLPPAR EQLNLRESAT ITCLVTGFSP ADVFVQWMQR GQPLSPEKYV | 300 |
| | | TSAPMPEPQA PGRYFAHSIL TVSEEEWNTG ETYTCVVAHE ALPNRVTERT VDKSTGKPTL | 360 |
| | | YNVSLVMSDT AGTCY | 375 |
| 7 | gi: 300621695 | MEFGLSWLFL VAILKGVQCE VQLLESGGGL VQPGGSLRLS CAASGFTFSS YAMSWVRQAP | 60 |
| | CBU30464.1 | GKGLEWVSAI SGSGYTTYYA DSVKGRFTIS RDNSKNTLYL QMNSLRAEDT AVYYCAKKPG | 120 |
| | | DYGSGSYYLD YWGQGTLVTV SSGSASAPTL FPLVSCENSP SDTSSVAVGC LAQDFLPDSI | 180 |
| | | TFSWKYKNNS DISSTRGFPS VLRGGKYAAT SQVLLPSKDV MQGTDEHVVC KVQHPNGNKE | 240 |
| | | KNVPLPVIAE LPPKVSVFVP PRDGFFGNPR KSKLICQATG FSPRQIQVSW LREGKQVGSG | 300 |
| | | VTTDQVQAEA KESGPTTYKV TSTLTIKESD WLSQSMFTCR VDHRGLTFQQ NASSMCVPDQ | 360 |
| | | DTAIRVFAIP PSFASIFLTK STKLTCLVTD LTTYDSVTIS WTRQNGEAVK THTNISESHP | 420 |
| | | NATFSAVGEA SICEDDWNSG ERFTCTVTHT DLPSPLKQTI SRPKGVALHR PDVYLLPPAR | 480 |
| | | EQLNLRESAT ITCLVTGFSP ADVFVQWMQR GQPLSPEKYV TSAPMPEPQA PGRYFAHSIL | 540 |
| | | TVSEEEWNTG ETYTCVVAHE ALPNRVTERT VDKSTGKPTL YNVSLVMSDT AGTCY | 595 |
| 8 | gi: 1335098 | TPLPPTSAHG NVAEGETKPD PDVTERCSDG WSFDATTLDD NGTMLFFKGE FVWKSHKWDR | 60 |
| | CAA26382.1 | ELISERWKNF PSPVDAAFRQ GHNSVFLIKG DKVWVYPPEK KEKGYPKLLQ DEFPGIPSPL | 120 |
| | | DAAVECHRGE CQAEGVLFFQ GDREWFWDLA TGTMKERSWP AVGNCSSALR WLGRYYCFQG | 180 |
| | | NQFLRFDPVR GEVPPRYPRD VRDYFMPCPG RGHGHRNGTG HGNSTHHGPE YMRCSPHLVL | 240 |
| | | SALTSDNHGA TYAFSGTHYW RLDTSRDGWH SWPIAHQWPQ GPSAVDAAFS WEEKLYLVQG | 300 |
| | | TQVYVFLTKG GYTLVSGYPK RLEKEVGTPH GIILDSVDAA FICPGSSRLH IMAGRRLWWL | 360 |
| | | DLKSGAQATW TELPWPHEKV DGALCMEKSL GPNSCSANGP GLYLIHGPNL YCYSDVEKLN | 420 |
| | | AAKALPQPQN VTSLLGCTH | 439 |
| 9 | gi: 10434804 | MEPRAVGVSK QDIREQIWGY MESQNLADFP RPVHHRIPNF KGSYLACQNI KDLDVFARAQ | 60 |
| | BAB14383.1 | EVKVDPDKPL EGVRLLVLQS KKTLLVPTPR LRTGLFNKIT PPPGATKDIL RKCATSQGVR | 120 |
| | | NYSVPIGLDS RVLVDLVVVG SVAASEKGWR IGKGEGYADL EYAMMVSMGA VSKETPVVTI | 180 |
| | | VHDCQVVDIP EELVEEHDIT VDYILTPTRV IATGCKRPKP MGITWFKISL EMMEKIPILR | 240 |
| | | SLRAREQQAG KDVTLQGEHQ HLPEPGCQQT VPLSVGRRPP DTPGPETNSM EAAPGSPPGE | 300 |
| | | GAPLAADVYV GNLPRDARVS DLKRALRELG SVPLRLTWQG PRRRAFLHYP DSAAASRPSP | 360 |
| | | ACRACAWAPT P | 371 |
| 10 | gi: 221044726 | MARVLGAPVA LGLWSLCWSL AIATPLPPTS AHGNVAEGET KPDPDVTERC SDGWSFDATT | 60 |
| | BAH14040.1 | LDDNGTMLFF KGEFVWKSHK WDRELISERL KNFPSPVDAA FRQGHNSVFL IKVLLGQNQG | 120 |
| | | QAGKGWNRHW GPFPQMALAW SP | 142 |
| 11 | gi: 215415638 | Same as KH3 | |
| | CAT02161.1 | | |
| 12 | gi: 189066554 | MAHVRGLQLP GCLALAALCS LVHSQHVFLA PQQARSLLQR VRRANTFLEE VRKGNLEREC | 60 |
| | BAG35804.1 | VEETCSYEEA FEALESSTAT DVFWAKYTAC ETARTPRDKL AACLEGNCAE GLGTNYRGHV | 120 |
| | | NITRSGIECQ LWRSRYPHKP EINSTTHPGA DLQENFCRNP DSSTMGPWCY TTDPTVRRQE | 180 |
| | | CSIPVCGQDQ VTVAMTPRSE GSSVNLSPPL EQCVPDRGQQ YQGRLAVTTH GLPCLAWASA | 240 |
| | | QAKALSKHQD FNSAVQLVEN FCRNPDGDEE GVWCYVAGKP GDFGYCDLNY CEEAVEEETG | 300 |
| | | DGLDEDSDRA IEGRTATSEY QTFFNPRTFG SGEADCGLRP LFEKKSLEDK TERELLESYI | 360 |
| | | DGRIVEGSDA EIGMSPWQVM LFRKSPQELL CGASLISDRW VLTAAHCLLY PPWDKNFTEN | 420 |
| | | DLLVRIGKHS RTRYERNIEK ISMLEKIYIH PRYNWRENLD RDIALMKLKK PVAFSDYIHP | 480 |
| | | VCLPDRETAA SLLQAGYKGR VTGWGNLKET WTANVGKGQP SVLQVVNLPI VERPVCKDST | 540 |
| | | RIRITDNMFC AGYKPDEGKR GDACEGDSGG PFVMKSPFNN RWYQMGIVSW GEGCDRDGKY | 600 |
| | | GFYTHVFRLK KWIQKVIDQF GE | 622 |
| 13 | gi: 194391084 | MKLSLTQESQ SEEIDCNDKD LFKAVDAALK KYNSQNQSNN QFVLYRITEA TKTVGSDTFY | 60 |
| | BAG60660.1 | SFKYEIKEGD CPVQSGKTWQ DCEYKDAAKA ATGECTATVG KRSSTKFSVA TQTCQITPAE | 120 |
| | | GPVVTAQYDC LGCVHPISTQ SPDLEPILRH GIQYFNNNTQ HSSLFMLNEV KRAQRQVVAG | 180 |
| | | LNFRITYSIV QTNCSKENFL FLTPDCKSLW NGDTGECTDN AYIDIQLRIA SFSQNCDIYP | 240 |
| | | GKDFVQPPTK ICVGCPRDIP TNSPELEETL THTITKLNAE NNATFYFKID NVKKARVQVV | 300 |
| | | AGKKYFIDFV ARETTCSKES NEELTESCET KKLGQSLDCN AEVYVVPWEK KIYPTVNCQP | 360 |
| | | LGMISLMKRP PGFSPFRSSR IGEIKEETTS HLRSCEYKGR PPKAGAEPAS EREVS | 415 |
| 14 | gi: 158255114 | MKLITILFLC SRLLLSLTQE SQSEEIDCND KDFFKAVDAA LKKYNSQNQS NNQFVLYRIT | 60 |
| | BAF83528.1 | EATKTVGSDT FYSFKYEIKE GDCPVQSGKT WQDCEYKDAA KAATGECTAT VGKRSSTKFS | 120 |
| | | VATQTCQITP AEGPVVTAQY DCLGCVHPIS TQSPDLEPIL RHGIQYFNNN TQHSSLFMLN | 180 |
| | | EVKRAQRQVV AGLNFRITYS IVQTNCSKEN FLFLTPDCKS LWNGDTGECT DNAYIDIQLR | 240 |
| | | IASFSQNCDI YPGKDFVQPP TKICVGCPRD IPTNSPELEE TLTHTITKLN AENNATFYFK | 300 |
| | | IDNVKKARVQ AVAGKKYFID FVARETTCSK ESNEELTESC ETKKLGQSLD CNAEVYVVPW | 360 |
| | | EKKIYPTVNC QPLGMISLMK RPPGFSPFRS SRIGEIKEET TSHLRSCEYK GRPPKAGAEP | 420 |
| | | ASEREVS | 427 |
| 15 | gi: 213506121 | MKLITILFLC SRLLLSLTQE SQSEEIDCND KDLFKAVDAA LKKYNSQNQS NNQFVLYRIT | 60 |
| | CAS91511.1 | EATKTVGSDT FYSFKYEIKE GDCPVQSGKT WQDCEYKDAA KAATGECTAT VGKRSSTKFS | 120 |
| | | VATQTCQITP AEGPVVTAQY DCLGCVHPIS TQSPDLEPIL RHGIQYFNNN TQHSSLFMLN | 180 |
| | | EVKRAQRQVV AGLNFRMTYS IVQTNCSKEN FLFLTPDCKS LWNGDTGECT DNAYIDIQLR | 240 |
| | | IASFSQNCDI YPGKDFVQPP TKICVGCPRD IPTNSPELEE TLTHTITKLN AENNATFYFK | 300 |
| | | IDNVKKARVQ VVAGKKYFID FVARETTCSK ESNEELTESC ETKKLGQSLD CNAEVYVVPW | 360 |
| | | EKKIYPTVNC QPLGMISLMK RPPGFSPFRS SRIGEIKEET TSHLRSCEYK GRPPKAGAEP | 420 |
| | | ASEREVS | 427 |
| 16 | gi: 213506103 | MKLITILFLC SRLLLSLTQE SQSEEIDCND KDLFKAVDAA LKKYNSQNQS NNQFVLYRIT | 60 |
| | CAS91502.1 | EATKTVGSDT FYSFKYEIKE GDCPVQSGKT WQDCEYKDAA KAATGECTAT VGKRSSTKFS | 120 |
| | | VATQTCQITP AEGPVVTAQY DCLGCVHPIS TQSPDLEPIL RHGIQYFNNN TQHSSLFMLN | 180 |
| | | EVKRAQRQVV AGLNFRMTYS IVQTNCSKEN FLFLTPDCKS LWNGDTGECT DNAYIDIQLR | 240 |
| | | IASFSQNCDI YPGKDFVQPP TKICVGCPRD IPTNSPELEE TLTHTITKLN AENNATFYFK | 300 |
| | | IDNVKKARVQ VVAGKKYFID FVARETTCSK ESNEELTESC ETKKLGQSLD CNAEVYVVPW | 360 |
| | | EKKIYPTVNC QPLGMISLMK RPPGFSPFRS SRIGEIKEET TSHLRSCEYK GRPPKAGAEP | 420 |
| | | ASEREVS | 427 |
| 17 | gi: 194376310 | MDDDIAALVV DNGSGMCKAG FAGDDAPRAV FPSIVGRPRH QGVMVGIVTN WDDMEKIWHH | 60 |
| | BAG62914.1 | TFYNELRVAP EEHPVLLTEA PLNPKANREK MTQIMFETFN TPAMYVAIQA VLSLYASGRT | 120 |
| | | TGIVMDSGDG VTHTVPIYEG YALPHAILRL DLAGRDLTDY LMKILTERGY SFTTTAEREI | 180 |
| | | VRDIKEKLCY VALDFEQEMA TAASSSSLEK SYELPDGQVI TIGNERFRCP EALFQPSFLG | 240 |
| | | MESCGIHETT FNSIMKCDVD IRKDLYANTV LSGGTTMYPG IADRMQKEIT ALAPSTMKIK | 300 |
| | | IIAPPERKYS VWIGGSILAS LSTFQQMWIS KQEYDESGPS IVHRKCF | 347 |
| 18 | gi: 194388064 | MEEEIAALVI DNGSGMCKAG FAGDDAPRAV FPSIVGRPRH QGVMVGMGQK DSYVGDEAQS | 60 |
| | BAG65416.1 | KRGILTLKYP IEHGIVTNWD DMEKIWHHTF YNELRVAPEE HPVLLTEAPL NPKANREKMT | 120 |
| | | QIMFETFNTT GIVMDSGDGV THTVPIYEGY ALPHAILRLD LAGRDLTDYL MKILTERGYS | 180 |
| | | FTTTAEREIV RDIKEKLCYV ALDFEQEMAT AASSSSLEKS YELPDGQVIT IGNERFRCPE | 240 |
| | | ALFQPSFLGM ESCGIHETTF NSIMKCDVDI RKDLYANTVL SGGTTMYPGI ADRMQKEITA | 300 |
| | | LAPSTMKIKI IAPPERKYSV WIGGSILASL STFQQMWISK QEYDESGPSI VHRKCF | 356 |
| 19 | IPI00964149 | MQKSEGSGGT QLKNRATGNY DQRTSSSTQL KHRNAVQGSK SSLSTSSPES ARKLHPRPSD | 60 |
| | gi: 126215685 | KLNPKTINPF GEQSRVPSAF AAIYSKGGIP CRLVHGSVKH RLQWECPPES LSFDPLLITL | 120 |
| | Q8N7B6.2 | AEGLRETKHP YTFVSKEGFR ELLLVKGAPE KAIPLLPRLI PVLKAALVHS DDEVFERGLN | 180 |
| | | ALVQLSVVVG PSLNDHLKHL LTSLSKRLMD KKFKEPITSA LQKLEQHGGS GSLSIIKSKI | 240 |
| | | PTYCSICC | 248 |
| 20 | IPI00966721 | MASETEKTHA LLQTCSTESL ISSLGLGAFC LVADRLLQFS TIQQNDWLRA LSDNAVHCVI | 60 |
| | gi: 121940485 | GMWSWAVVTG IKKKTDFGEI ILAGFLASVI DVDHFFLAGS MSLKAALTLP RRPFLHCSTV | 120 |
| | Q0VDI3.1 | IPVVVLTLKF TMHLFKLKDS WCFLPWMLFI SWTSHHIRDG IRHGLWICPF GKTSPLPFWL | 180 |
| | | YVIITSSLPH ICSFVMYLTG TRQMMSSKHG VRIDV | 215 |
| 21 | IPI00966826 | MDFTAQPKPA TALCGVVSAD GKIAYPPGVK EITDKITTDE MIKRLKMVVK TFMDMDQDSE | 60 |
| | gi: 121947590 | DEKQQYLPLA LHLASEFFLR NPNKDVRLLV ACCLADIFRI YAPEAPYTSH DKLKDIFLFI | 120 |
| | Q29RF7.1 | TRQLKGLEDT KSPQFNRYFY LLENLAWVKS YNICFELEDC NEIFIQLFRT LFSVINNSHN | 180 |
| | | KKVQMHMLDL MSSIIMEGDG VTQELLDSIL INLIPAHKNL NKQSFDLAKV LLKRTVQTIE | 240 |
| | | ACIANFFNQV LVLGRSSVSD LSEHVFDLIQ ELFAIDPHLL LSVMPQLEFK LKSNDGEERL | 300 |
| | | AVVRLLAKLF GSKDSDLATQ NRPLWQCFLG RFNDIHVPVR LESVKFASHC LMNHPDLAKD | 360 |
| | | LTEYLKVRSH DPEEAIRHDV IVTIITAAKR DLALVNDQLL GFVRERTLDK RWRVRKEAMM | 420 |
| | | GLAQLYKKYC LHGEAGKEAA EKVSWIKDKL LHIYYQNSID DKLLVEKIFA QYLVPHNLET | 480 |
| | | EERMKCLYYL YASLDPNAVK ALNEMWKCQN MLRSHVRELL DLHKQPTSEA NCSAMFGKLM | 540 |
| | | TIAKNLPDPG KAQDFVKKFN QVLGDDEKLR SQLELLISPT CSCKQADICV REIARKLANP | 600 |
| | | KQPTNPFLEM VKFLLERIAP VHIDSEAISA LVKLMNKSIE GTADDEEEGV SPDTAIRSGL | 660 |
| | | ELLKVLSFTH PTSFHSAETY ESLLQCLRME DDKVAEAAIQ IFRNTGHKIE TDLPQIRSTL | 720 |
| | | IPILHQKAKR GTPHQAKQAV HCIHAIFTNK EVQLAQIFEP LSRSLNADVP EQLITPLVSL | 780 |
| | | GHISMLAPDQ FASPMKSVVA NFIVKDLLMN DRSTGEKNGK LWSPDEEVSP EVLAKVQAIK | 840 |
| | | LLVRWLLGMK NNQSKSANST LRLLSAMLVS EGDLTEQKRI SKSDMSRLRL AAGSAIMKLA | 900 |
| | | QEPCYHEIIT PEQFQLCALV INDECYQVRQ IFAQKLHKAL VKLLLPLEYM AIFALCAKDP | 960 |
| | | VKERRAHARQ CLLKNISIRR EYIKQNPMAT EKLLSLLPEY VVPYMIHLLA HDPDFTRSQD | 1020 |
| | | VDQLRDIKEC LWFMLEVLMT KNENNSHAFM KKMAENIKLT RDAQSPDESK TNEKLYTVCD | 1080 |
| | | VALCVINSKS ALCNADSPKD PVLPMKFFTQ PEKDFCNDKS YISEETRVLL LTGKPKPAGV | 1140 |
| | | LGAVNKPLSA TGRKPYVRST GTETGSNINV NSELNPSTGN RSREQSSEAA ETGVSENEEN | 1200 |
| | | PVRIISVTPV KNIDPVKNKE INSDQATQGN ISSDRGKKRT VTAAGAENIQ QKTDEKVDES | 1260 |
| | | GPPAPSKPRR GRRPKSESQG NATKNDDLNK PINKGRKRAA VGQESPGGLE AGNAKAPKLQ | 1320 |
| | | DLAKKAAPAE RQIDLQR | 1337 |
| 22 | IPI00760788 | MAEEQEFTQL CKLPAQPSHP HCVNNTYRSA QHSQALLRGL LALRDSGILF DVVLVVEGRH | 60 |
| | gi: 109892504 | IEAHRILLAA SCDYFRGMFA GGLKEMEQEE VLIHGVSYNA MCQILHFIYT SELELSLSNV | 120 |
| | Q53GT1.2 | QETLVAACQL QIPEIIHFCC DFLMSWVDEE NILDVYRLAE LFDLSRLTEQ LDTYILKNFV | 180 |
| | | AFSRTDKYRQ LPLEKVYSLL SSNRLEVSCE TEVYEGALLY HYSLEQVQAD QISLHEPPKL | 240 |
| | | LETVRFPLME AEVLQRLHDK LDPSPLRDTV ASALMYHRNE SLQPSLQSPQ TELRSDFQCV | 300 |
| | | VGFGGIHSTP STVLSDQAKY LNPLLGEWKH FTASLAPRMS NQGIAVLNNF VYLIGGDNNV | 360 |
| | | QGFRAESRCW RYDPRHNRWF QIQSLQQEHA DLSVCVVGRY IYAVAGRDYH NDLNAVERYD | 420 |
| | | PATNSWAYVA PLKREVYAHA GATLEGKMYI TCGRRGEDYL KETHCYDPGS NTWHTLADGP | 480 |
| | | VRRAWHGMAT LLNKLYVIGG SNNDAGYRRD VHQVACYSCT SGQWSSVCPL PAGHGEPGIA | 540 |
| | | VLDNRIYVLG GRSHNRGSRT GYVHIYDVEK DCWEEGPQLD NSISGLAACV LTLPRSLLLE | 600 |
| | | PPRGTPDRSQ ADPDFASEVM SVSDWEEFDN SSED | 634 |
| 23 | IPI00917278 | MKQLQPQPPP KMGDFYDPEH PTPEEEENEA KIENVQKTGF IKGPMFKGVA SSRFLPKGTK | 60 |
| | gi: | TKVNLEEQGR QKVSFSFSLT KKTLQNRFLT ALGNEKQSDT PNPPAVPLQV DSTPKMKMEI | 120 |
| | | GDTLSTAEES SPPKSRVELG KIHFKKHLLH VTSRPLLATT TAVASPPTHA APLPAVIAES | 180 |
| | | TTVDSPPSSP PPPPPPAQAT TLSSPAPVTE PVALPHTPIT VLMAAPVPLP VDVAVRSLKE | 240 |
| | | PPIIIVPESL EADTKQDTIS NSLEEHVTQI LNEQADISSK KEDSHIGKDE EIPDSSKISL | 300 |
| | | SCKKTGSKKK SSQSEGIFLG SESDEDSVRT SSSQRSHDLK FSASIEKERD FKKSSAPLKS | 360 |
| | | EDLGKPSRSK TDRDDKYFSY SKLERDTRYV SSRCRSERER RRSRSHSRSE RGSRTNLSYS | 420 |
| | | RSERSHYYDS DRRYHRSSPY RERTRYSRPY TDNRARESSD SEEEYKKTYS RRTSSHSSSY | 480 |
| | | RDLRTSSYSK SDRDCKTETS YLEMERRGKY SSKLERESKR TSENEAIKRC CSPPNELGFR | 540 |
| | | RGSSYSKHDS SASRYKSTLS KPIPKSDKFK NSFCCTELNE EIKQSHSFSL QTPCSKGSEL | 600 |
| | | RMINKNPERE KAGSPAPSNR LNDSPTLKKL DELPIFKSEF ITHDSHDSIK ELDSLSKVKN | 660 |
| | | DQLRSFCPIE LNINGSPGAE SDLATFCTSK TDAVLMTSDD SVTGSELSPL VKACMLSSNG | 720 |
| | | FQNISRCKEK DLDDTCMLHK KSESPFRETE PLVSPHQDKL MSMPVMTVDY SKTVVKEPVD | 780 |
| | | TRVSCCKTKD SDIYCTLNDS NPSLCNSEAE NIEPSVMKIS SNSFMNVHLE SKPVICDSRN | 840 |
| | | LTDHSKFACE EYKQSIGSTS SASVNHFDDL YQPIGSSGIA SSLQSLPPGI KVDSLTLLKC | 900 |
| | | GENTSPVLDA VLKSKKSSEF LKHAGKETIV EVGSDLPDSG KGFASRENRR NNGLSGKCLQ | 960 |
| | | EAQEEGNSIL PERRGRPEIS LDERGEGGHV HTSDDSEVVF SSCDLNLTME DSDGVTYALK | 1020 |
| | | CDSSGHAPEI VSTVHEDYSG SSESSNDESD SEDTDSDDSS IPRNRLQSVV VVPKNSTLPM | 1080 |
| | | EETSPCSSRS SQSYRHYSDH WEDERLESRR HLYEEKFESI ASKACPQTDK FFLHKGTEKN | 1140 |
| | | PEISFTQSSR KQIDNRLPEL SHPQSDGVDS TSHTDVKSDP LGHPNSEETV KAKIPSRQQE | 1200 |
| | | ELPIYSSDFE DVPNKSWQQT TFQNRPDSRL GKTELSFSSS CEIPHVDGLH SSEELRNLGW | 1260 |
| | | DFSQEKPSTT YQQPDSSYGA CGGHKYQQNA EQYGGTRDYW QGNGYWDPRS GRPPGTGVVY | 1320 |
| | | DRTQGQVPDS LTDDREEEEN WDQQDGSHFS DQSDKFLLSL QKDKGSVQAP EISSNSIKDT | 1380 |
| | | LAVNEKKDFS KNLEKNDIKD RGPLKKRRQE IESDSESDGE LQDRKKVRVE VEQGETSVPP | 1440 |
| | | GSALVGPSCV MDDFRDPQRW KECAKQGKMP CYFDLIEENV YLTERKKNKS HRDIKRMQCE | 1500 |
| | | CTPLSKDERA QGEIACGEDC LNRLLMIECS SRCPNGDYCS NRRFQRKQHA DVEVILTEKK | 1560 |
| | | GWGLRAAKDL PSNTFVLEYC GEVLDHKEFK ARVKEYARNK NIHYYFMALK NDEIIDATQK | 1620 |
| | | GNCSRFMNHS CEPNCETQKW TVNGQLRVGF FTTKLVPSGS ELTFDYQFQR YGKEAQKCFC | 1680 |
| | | GSANCRGYLG GENRVSIRAA GGKMKKERSR KKDSVDGELE ALMENGEGLS DKNQVLSLSR | 1740 |
| | | LMVRIETLEQ KLTCLELIQN THSQSCLKSF LERHGLSLLW IWMAELGDGR ESNQKLQEEI | 1800 |
| | | IKTLEHLPIP TKNMLEESKV LPIIQRWSQT KTAVPPLSEG DGYSSENTSR AHTPLNTPDP | 1860 |
| | | STKLSTEADT DTPKKLMFRR LKIISENSMD SAISDATSEL EGKDGKEDLD QLENVPVEEE | 1920 |
| | | EELQSQQLLP QQLPECKVDS ETNIEASKLP TSEPEADAEI EPKESNGTKL EEPINEETPS | 1980 |
| | | QDEEEGVSDV ESERSQEQPD KTVDISDLAT KLLDSWKDLK EVYRIPKKSQ TEKENTTTER | 2040 |
| | | GRDAVGFRDQ TPAPKTPNRS RERDPDKQTQ NKEKRKRRSS LSPPSSAYER GTKRPDDRYD | 2100 |
| | | TPTSKKKVRI KDRNKLSTEE RRKLFEQEVA QREAQKQQQQ MQNLGMTSPL PYDSLGYNAP | 2160 |
| | | HHPFAGYPPG YPMQAYVDPS NPNAGKVLLP TPSMDPVCSP APYDHAQPLV GHSTEPLSAP | 2220 |
| | | PPVPVVPHVA APVEVSSSQY VAQSDGVVHQ DSSVAVLPVP APGPVQGQ | 2268 |
| 24 | IPI00966721 | Same as KH 20 | |
| | gi: 121940485 | | |
| | Q0VDI3.1 | | |
| 25 | IPI01012037 | MNGEYRGRGF GRGRFQSWKR GRGGGNFSGK WREREHRPDL SKTTGKRTSE QTPQFLLSTK | 60 |
| | gi: 74735024 | TPQSMQSTLD RFIPYKGWKL YFSEVYSDSS PLIEKIQAFE KFFTRHIDLY DKDEIERKGS | 120 |
| | Q9UHY7.1 | ILVDFKELTE GGEVTNLIPD IATELRDAPE KTLACMGLAI HQVLTKDLER HAAELQAQEG | 180 |
| | | LSNDGETMVN VPHIHARVYN YEPLTQLKNV RANYYGKYIA LRGTVVRVSN IKPLCTKMAF | 240 |
| | | LCAACGEIQS FPLPDGKYSL PTKCPVPVCR GRSFTALRSS PLTVTMDWQS IKIQELMSDD | 300 |
| | | QREAGRIPRT IECELVHDLV DSCVPGDTVT ITGIVKVSNA EEGSRNKNDK CMFLLYIEAN | 360 |
| | | SISNSKGQKT KSSEDGCKHG MLMEFSLKDL YAIQEIQAEE NLFKLIVNSL CPVIFGHELV | 420 |
| | | KAGLALALFG GSQKYADDKN RIPIRGDPHI LVVGDPGLGK SQMLQAACNV APRGVYVCGN | 480 |
| | | TTTTSGLTVT LSKDSSSGDF ALEAGALVLG DQGICGIDEF DKMGNQHQAL LEAMEQQSIS | 540 |
| | | LAKAGVVCSL PARTSIIAAA NPVGGHYNKA KTVSENLKMG SALLSRFDLV FILLDTPNEH | 600 |
| | | HDHLLSEHVI AIRAGKQRTI SSATVARMNS QDSNTSVLEV VSEKPLSERL KVVPGETIDP | 660 |
| | | IPHQLLRKYI GYARQYVYPR LSTEAARVLQ DFYLELRKQS QRLNSSPITT RQLESLIRLT | 720 |
| | | EARARLELRE EATKEDAEDI VEIMKYSMLG TYSDEFGNLD FERSQHGSGM SNRSTAKRFI | 780 |
| | | SALNNVAERT YNNIFQFHQL RQIAKELNIQ VADFENFIGS LNDQGYLLKK GPKVYQLQTM | 840 |
| 26 | IPI00940730 | MVVLSVPAEV TVILLDIEGT TTPIAFVKDI LFPYIEENVK EYLQTHWEEE ECQQDVSLLR | 60 |
| | gi: | KQAEEDAHLD GAVPIPAASG NGVDDLQQMI QAVVDNVCWQ MSLDRKTTAL KQLQGHMWRA | 120 |
| | | AFTAGRMKAE FFADVVPAVR KWREAGMKVY IYSSGSVEAQ KLLFGHSTEG DILELVDGHF | 180 |
| | | DTKIGHKVES ESYRKIADSI GCSTNNILFL TDVTREASAA EEADVHVAVV VRPGNAGLTD | 240 |
| | | DEKTYYSLIT SFSELYLPSS T | 261 |
| 27 | IPI00977191 | MAMESTATAA VAAELVSADK IEDVPAPSTS ADKVESLDVD SEAKKLLGLG QKHLVMGDIP | 60 |
| | gi: 23503077 | AAVNAFQEAA SLLGKKYGET ANECGEAFFF YGKSLLELAR MENGVLGNAL EGVHVEEEEG | 120 |
| | P49321.2 | EKTEDESLVE NNDNIDEEAR EELREQVYDA MGEKEEAKKT EDKSLAKPET DKEQDSEMEK | 180 |
| | | GGREDMDISK SAEEPQEKVD LTLDWLTETS EEAKGGAAPE GPNEAEVTSG KPEQEVPDAE | 240 |
| | | EEKSVSGTDV QEECREKGGQ EKQGEVIVSI EEKPKEVSEE QPVVTLEKQG TAVEVEAESL | 300 |
| | | DPTVKPVDVG GDEPEEKVVT SENEAGKAVL EQLVGQEVPP AEESPEVTTE AAEASAVEAG | 360 |
| | | SEVSEKPGQE APVLPKDGAV NGPSVVGDQT PIEPQTSIER LTETKDGSGL EEKVRAKLVP | 420 |
| | | SQEETKLSVE ESEAAGDGVD TKVAQGATEK SPEDKVQIAA NEETQEREEQ MKEGEETEGS | 480 |
| | | EEDDKENDKT EEMPNDSVLE NKSLQENEEE EIGNLELAWD MLDLAKIIFK RQETKEAQLY | 540 |
| | | AAQAHLKLGE VSVESENYVQ AVEEFQSCLN LQEQYLEAHD RLLAETHYQL GLAYGYNSQY | 600 |
| | | DEAVAQFSKS IEVIENRMAV LNEQVKEAEG SSAEYKKEIE ELKELLPEIR EKIEDAKESQ | 660 |
| | | RSGNVAELAL KATLVESSTS GFTPGGGGSS VSMIASRKPT DGASSSNCVT DISHLVRKKR | 720 |
| | | KPEEESPRKD DAKKAKQEPE VNGGSGDAVP SGNEVSENME EEAENQAESR AAVEGTVEAG | 780 |
| | | ATVESTAC | 788 |
| 28 | IPI00022434 | MKWVTFISLL FLFSSAYSRG VFRRDAHKSE VAHRFKDLGE ENFKALVLIA FAQYLQQCPF | 60 |
| | gi: 113576 | EDHVKLVNEV TEFAKTCVAD ESAENCDKSL HTLFGDKLCT VATLRETYGE MADCCAKQEP | 120 |
| | P02768.2 | ERNECFLQHK DDNPNLPRLV RPEVDVMCTA FHDNEETFLK KYLYEIARRH PYFYAPELLF | 180 |
| | | FAKRYKAAFT ECCQAADKAA CLLPKLDELR DEGKASSAKQ RLKCASLQKF GERAFKAWAV | 240 |
| | | ARLSQRFPKA EFAEVSKLVT DLTKVHTECC HGDLLECADD RADLAKYQKF NQDSISSKLK | 300 |
| | | ECCEKPLLEK SHCIAEVEND EMPADLPSLA ADFVESKDVC KNYAEAKDVF LGMFLYEYAR | 360 |
| | | RHPDYSVVLL LRLAKTYETT LEKCCAAADP HECYAKVFDE FKPLVEEPQN LIKQNCELFE | 420 |
| | | QLGEYKFQNA LLVRYTKKVP QVSTPTLVEV SRNLGKVGSK CCKHPEAKRM PCAEDYLSVV | 480 |
| | | LNQLCVLHEK TPVSDRVTKC CTESLVNRRP CFSALEVDET YVPKEFNAET FTFHADICTL | 540 |
| | | SEKERQIKKQ TALVELVKHK PKATKEQLKA VMDDFAAFVE KCCKADDKET CFAEEGKKLV | 600 |
| | | AASQAALGL | 609 |
| 29 | IPI00022434 | Same as KH 28 | |
| | gi: 113576 | | |
| | P02768.2 | | |
| 30 | IPI00219713 | MSWSLHPRNL ILYFYALLFL SSTCVAYVAT RDNCCILDER FGSYCPTTCG IADFLSTYQT | 60 |
| | gi: 20178280 | KVDKDLQSLE DILHQVENKT SEVKQLIKAI QLTYNPDESS KPNMIDAATL KSRKMLEEIM | 120 |
| | P02679 | KYEASILTHD SSIRYLQEIY NSNNQKIVNL KEKVAQLEAQ CQEPCKDTVQ IHDITGKDCQ | 180 |
| | | DIANKGAKQS GLYFIKPLKA NQQFLVYCEI DGSGNGWTVF QKRLDGSVDF KKNWIQYKEG | 240 |
| | | FGHLSPTGTT EFWLGNEKIH LISTQSAIPY ALRVELEDWN GRTSTADYAM FKVGPEADKY | 300 |
| | | RLTYAYFAGG DAGDAFDGFD FGDDPSDKFF TSHNGMQFST WDNDNDKFEG NCAEQDGSGW | 360 |
| | | WMNKCHAGHL NGVYYQGGTY SKASTPNGYD NGIIWATWKT RWYSMKKTTM KIIPFNRLTI | 420 |
| | | GEGQQHHLGG AKQVRPEHPA ETEYDSLYPE DDL | 453 |
| 31 | IPI00219713 | Same as KH 30 | |
| | gi: 20178280 | | |
| | P02679 | | |
| 32 | IPI00220327 | MSRQFSSRSG YRSGGGFSSG SAGIINYQRR TTSSSTRRSG GGGGRFSSCG GGGGSFGAGG | 60 |
| | gi: 238054406 | GFGSRSLVNL GGSKSISISV ARGGGRGSGF GGGYGGGGFG GGGFGGGGFG GGGIGGGGFG | 120 |
| | P04264.6 | GFGSGGGGFG GGGFGGGGYG GGYGPVCPPG GIQEVTINQS LLQPLNVEID PEIQKVKSRE | 180 |
| | | REQIKSLNNQ FASFIDKVRF LEQQNQVLQT KWELLQQVDT STRTHNLEPY FESFINNLRR | 240 |
| | | RVDQLKSDQS RLDSELKNMQ DMVEDYRNKY EDEINKRTNA ENEFVTIKKD VDGAYMTKVD | 300 |
| | | LQAKLDNLQQ EIDFLTALYQ AELSQMQTQI SETNVILSMD NNRSLDLDSI IAEVKAQYED | 360 |
| | | IAQKSKAEAE SLYQSKYEEL QITAGRHGDS VRNSKIEISE LNRVIQRLRS EIDNVKKQIS | 420 |
| | | NLQQSISDAE QRGENALKDA KNKLNDLEDA LQQAKEDLAR LLRDYQELMN TKLALDLEIA | 480 |
| | | TYRTLLEGEE SRMSGECAPN VSVSVSTSHT TISGGGSRGG GGGGYGSGGS SYGSGGGSYG | 540 |
| | | SGGGGGGGRG SYGSGGSSYG SGGGSYGSGG GGGGHGSYGS GSSSGGYRGG SGGGGGGSSG | 600 |
| | | GRGSGGGSSG GSIGGRGSSS GGVKSSGGSS SVKFVSTTYS GVTR | 644 |
| 33 | IPI00029739 | MRLLAKIICL MLWAICVAED CNELPPRRNT EILTGSWSDQ TYPEGTQAIY KCRPGYRSLG | 60 |
| | gi: 158517847 | NVIMVCRKGE WVALNPLRKC QKRPCGHPGD TPFGTFTLTG GNVFEYGVKA VYTCNEGYQL | 120 |
| | P08603.4 | LGEINYRECD TDGWTNDIPI CEVVKCLPVT APENGKIVSS AMEPDREYHF GQAVRFVCNS | 180 |
| | | GYKIEGDEEM HCSDDGFWSK EKPKCVEISC KSPDVINGSP ISQKIIYKEN ERFQYKCNMG | 240 |
| | | YEYSERGDAV CTESGWRPLP SCEEKSCDNP YIPNGDYSPL RIKHRTGDEI TYQCRNGFYP | 300 |
| | | ATRGNTAKCT STGWIPAPRC TLKPCDYPDI KHGGLYHENM RRPYFPVAVG KYYSYYCDEH | 360 |
| | | FETPSGSYWD HIHCTQDGWS PAVPCLRKCY FPYLENGYNQ NYGRKFVQGK SIDVACHPGY | 420 |
| | | ALPKAQTTVT CMENGWSPTP RCIRVKTCSK SSIDIENGFI SESQYTYALK EKAKYQCKLG | 480 |
| | | YVTADGETSG SITCGKDGWS AQPTCIKSCD IPVFMNARTK NDFTWFKLND TLDYECHDGY | 540 |
| | | ESNTGSTTGS IVCGYNGWSD LPICYERECE LPKIDVHLVP DRKKDQYKVG EVLKFSCKPG | 600 |
| | | FTIVGPNSVQ CYHFGLSPDL PICKEQVQSC GPPPELLNGN VKEKTKEEYG HSEVVEYYCN | 660 |
| | | PRFLMKGPNK IQCVDGEWTT LPVCIVEEST CGDIPELEHG WAQLSSPPYY YGDSVEFNCS | 720 |
| | | ESFTMIGHRS ITCIHGVWTQ LPQCVAIDKL KKCKSSNLII LEEHLKNKKE FDHNSNIRYR | 780 |
| | | CRGKEGWIHT VCINGRWDPE VNCSMAQIQL CPPPPQIPNS HNMTTTLNYR DGEKVSVLCQ | 840 |
| | | ENYLIQEGEE ITCKDGRWQS IPLCVEKIPC SQPPQIEHGT INSSRSSQES YAHGTKLSYT | 900 |
| | | CEGGFRISEE NETTCYMGKW SSPPQCEGLP CKSPPEISHG VVAHMSDSYQ YGEEVTYKCF | 960 |
| | | EGFGIDGPAI AKCLGEKWSH PPSCIKTDCL SLPSFENAIP MGEKKDVYKA GEQVTYTCAT | 1020 |
| | | YYKMDGASNV TCINSRWTGR PTCRDTSCVN PPTVQNAYIV SRQMSKYPSG ERVRYQCRSP | 1080 |
| | | YEMFGDEEVM CLNGNWTEPP QCKDSTGKCG PPPPIDNGDI TSFPLSVYAP ASSVEYQCQN | 1140 |
| | | LYQLEGNKRI TCRNGQWSEP PKCLHPCVIS REIMENYNIA LRWTAKQKLY SRTGESVEFV | 1200 |
| | | CKRGYRLSSR SHTLRTTCWD GKLEYPTCAK R | 1231 |
| 34 | IPI00384853 | QAHGRCSAGAQFVFCRRSAGAACTQQALSR (Sequence 59-88) | |
| | gi: | CLVGAQCVLSR (Sequence 100-110) | |
| | | CTVCTQQALSR (Sequence 125-135) | |
| 35 | IPI00479708 | GSASAPTLFP LVSCENSPSD TSSVAVGCLA QDFLPDSITL SWKYKNNSDI SSTRGFPSVL | 60 |
| | gi: 193806374 | RGGKYAATSQ VLLPSKDVMQ GTDEHVVCKV QHPNGNKEKN VPLPVIAELP PKVSVFVPPR | 120 |
| | P01871.3 | DGFFGNPRKS KLICQATGFS PRQIQVSWLR EGKQVGSGVT TDQVQAEAKE SGPTTYKVTS | 180 |
| | | TLTIKESDWL GQSMFTCRVD HRGLTFQQNA SSMCVPDQDT AIRVFAIPPS FASIFLTKST | 240 |
| | | KLTCLVTDLT TYDSVTISWT RQNGEAVKTH TNISESHPNA TFSAVGEASI CEDDWNSGER | 300 |
| | | FTCTVTHTDL PSPLKQTISR PKGVALHRPD VYLLPPAREQ LNLRESATIT CLVTGFSPAD | 360 |
| | | VFVQWMQRGQ PLSPEKYVTS APMPEPQAPG RYFAHSILTV SEEEWNTGET YTCVAHEALP | 420 |
| | | NRVTERTVDK STGKPTLYNV SLVMSDTAGT CY | 452 |
| 36 | IPI00298497 | MKRMVSWSFH KLKTMKHLLL LLLCVFLVKS QGVNDNEEGF FSARGHRPLD KKREEAPSLR | 60 |
| | gi: 399492 | PAPPPISGGG YRARPAKAAA TQKKVERKAP DAGGCLHADP DLGVLCPTGC QLQEALLQQE | 120 |
| | P02675.2 | RPIRNSVDEL NNNVEAVSQT SSSSFQYMYL LKDLWQKRQK QVKDNENVVN EYSSELEKHQ | 180 |
| | | LYIDETVNSN IPTNLRVLRS ILENLRSKIQ KLESDVSAQM EYCRTPCTVS CNIPVVSGKE | 240 |
| | | CEEIIRKGGE TSEMYLIQPD SSVKPYRVYC DMNTENGGWT VIQNRQDGSV DFGRKWDPYK | 300 |
| | | QGFGNVATNT DGKNYCGLPG EYWLGNDKIS QLTRMGPTEL LIEMEDWKGD KVKAHYGGFT | 360 |
| | | VQNEANKYQI SVNKYRGTAG NALMDGASQL MGENRTMTIH NGMFFSTYDR DNDGWLTSDP | 420 |
| | | RKQCSKEDGG GWWYNRCHAA NPNGRYYWGG QYTWDMAKHG TDDGVVWMNW KGSWYSMRKM | 480 |
| | | SMKIRPFFPQ Q | 491 |
| 37 | IPI00021841 | MKAAVLTLAV LFLTGSQARH FWQQDEPPQS PWDRVKDLAT VYVDVLKDSG RDYVSQFEGS | 60 |
| | gi: 113992 | ALGKQLNLKL LDNWDSVTST FSKLREQLGP VTQEFWDNLE KETEGLRQEM SKDLEEVKAK | 120 |
| | P02647.1 | VQPYLDDFQK KWQEEMELYR QKVEPLRAEL QEGARQKLHE LQEKLSPLGE EMRDRARAHV | 180 |
| | | DALRTHLAPY SDELRQRLAA RLEALKENGG ARLAEYHAKA TEHLSTLSEK AKPALEDLRQ | 240 |
| | | GLLPVLESFK VSFLSALEEY TKKLNTQ | 267 |
| 38 | IPI00783987 | MGPTSGPSLL LLLLTHLPLA LGSPMYSIIT PNILRLESEE TMVLEAHDAQ GDVPVTVTVH | 60 |
| | gi: 119370332 | DFPGKKLVLS SEKTVLTPAT NHMGNVTFTI PANREFKSEK GRNKFVTVQA TFGTQVVEKV | 120 |
| | P01024.2 | VLVSLQSGYL FIQTDKTIYT PGSTVLYRIF TVNHKLLPVG RTVMVNIENP EGIPVKQDSL | 180 |
| | | SSQNQLGVLP LSWDIPELVN MGQWKIRAYY ENSPQQVFST EFEVKEYVLP SFEVIVEPTE | 240 |
| | | KFYYIYNEKG LEVTITARFL YGKKVEGTAF VIFGIQDGEQ RISLPESLKR IPIEDGSGEV | 300 |
| | | VLSRKVLLDG VQNPRAEDLV GKSLYVSATV ILHSGSDMVQ AERSGIPIVT SPYQIHFTKT | 360 |
| | | PKYFKPGMPF DLMVFVTNPD GSPAYRVPVA VQGEDTVQSL TQGDGVAKLS INTHPSQKPL | 420 |
| | | SITVRTKKQE LSEAEQATRT MQALPYSTVG NSNNYLHLSV LRTELRPGET LNVNFLLRMD | 480 |
| | | RAHEAKIRYY TYLIMNKGRL LKAGRQVREP GQDLVVLPLS ITTDFIPSFR LVAYYTLIGA | 540 |
| | | SGQREVVADS VWVDVKDSCV GSLVVKSGQS EDRQPVPGQQ MTLKIEGDHG ARVVLVAVDK | 600 |
| | | GVFVLNKKNK LTQSKIWDVV EKADIGCTPG SGKDYAGVFS DAGLTFTSSS GQQTAQRAEL | 660 |
| | | QCPQPAARRR RSVQLTEKRM DKVGKYPKEL RKCCEDGMRE NPMRFSCQRR TRFISLGEAC | 720 |
| | | KKVFLDCCNY ITELRRQHAR ASHLGLARSN LDEDIIAEEN IVSRSEFPES WLWNVEDLKE | 780 |
| | | PPKNGISTKL MNIFLKDSIT TWEILAVSMS DKKGICVADP FEVTVMQDFF IDLRLPYSVV | 840 |
| | | RNEQVEIRAV LYNYRQNQEL KVRVELLHNP AFCSLATTKR RHQQTVTIPP KSSLSVPYVI | 900 |
| | | VPLKTGLQEV EVKAAVYHHF ISDGVRKSLK VVPEGIRMNK TVAVRTLDPE RLGREGVQKE | 960 |
| | | DIPPADLSDQ VPDTESETRI LLQGTPVAQM TEDAVDAERL KHLIVTPSGC GEQNMIGMTP | 1020 |
| | | TVIAVHYLDE TEQWEKFGLE KRQGALELIK KGYTQQLAFR QPSSAFAAFV KRAPSTWLTA | 1080 |
| | | YVVKVFSLAV NLIAIDSQVL CGAVKWLILE KQKPDGVFQE DAPVIHQEMI GGLRNNNEKD | 1140 |
| | | MALTAFVLIS LQEAKDICEE QVNSLPGSIT KAGDFLEANY MNLQRSYTVA IAGYALAQMG | 1200 |
| | | RLKGPLLNKF LTTAKDKNRW EDPGKQLYNV EATSYALLAL LQLKDFDFVP PVVRWLNEQR | 1260 |
| | | YYGGGYGSTQ ATFMVFQALA QYQKDAPDHQ ELNLDVSLQL PSRSSKITHR IHWESASLLR | 1320 |
| | | SEETKENEGF TVTAEGKGQG TLSVVTMYHA KAKDQLTCNK FDLKVTIKPA PETEKRPQDA | 1380 |
| | | KNTMILEICT RYRGDQDATM SILDISMMTG FAPDTDDLKQ LANGVDRYIS KYELDKAFSD | 1440 |
| | | RNTLIIYLDK VSHSEDDCLA FKVHQYFNVE LIQPGAVKVY AYYNLEESCT RFYHPEKEDG | 1500 |
| | | KLNKLCRDEL CRCAEENCFI QKSDDKVTLE ERLDKACEPG VDYVYKTRLV KVQLSNDFDE | 1560 |
| | | YIMAIEQTIK SGSDEVQVGQ QRTFISPIKC REALKLEEKK HYLMWGLSSD FWGEKPNLSY | 1620 |
| | | IIGKDTWVEH WPEEDECQDE ENQKQCQDLG AFTESMVVFG CPN | 1663 |
| 39 | IPI00878282 | MKWVTFISLL FLFSSAYSRG VFRRDAHKSE VAHRFKDLGE ENFKALVLIA FAQYLQQCPF | 60 |
| | gi: 113576 | EDHVKLVNEV TEFAKTCVAD ESAENCDKSL HTLFGDKLCT VATLRETYGE MADCCAKQEP | 120 |
| | P02768.2 | ERNECFLQHK DDNPNLPRLV RPEVDVMCTA FHDNEETFLK KYLYEIARRH PYFYAPELLF | 180 |
| | | FAKRYKAAFT ECCQAADKAA CLLPKLDELR DEGKASSAKQ RLKCASLQKF GERAFKAWAV | 240 |
| | | ARLSQRFPKA EFAEVSKLVT DLTKVHTECC HGDLLECADD RADLAKYICE NQDSISSKLK | 300 |
| | | ECCEKPLLEK SHCIAEVEND EMPADLPSLA ADFVESKDVC KNYAEAKDVF LGMFLYEYAR | 360 |
| | | RHPDYSVVLL LRLAKTYETT LEKCCAAADP HECYAKVFDE FKPLVEEPQN LIKQNCELFE | 420 |
| | | QLGEYKFQNA LLVRYTKKVP QVSTPTLVEV SRNLGKVGSK CCKHPEAKRM PCAEDYLSVV | 480 |
| | | LNQLCVLHEK TPVSDRVTKC CTESLVNRRP CFSALEVDET YVPKEFNAET FTFHADICTL | 540 |
| | | SEKERQIKKQ TALVELVKHK PKATKEQLKA VMDDFAAFVE KCCKADDKET CFAEEGKKLV | 600 |
| | | AASQAALGL | 609 |
| 40 | IPI00784842 | GRFTISGDISTNTLYLQMHSLR (Sequence 85-106) | |
| | gi: | TPEVTCVVVDVSHEDPEVKFNWYVDGVEVHNAK (Sequence 284-316) | |
| | | ALPAPIEK (Sequence 355-362) | |
| | | GQPREPQVYTLPPSRDELTKGFYPSDIAVEWESNGQPENNYK (Sequence 369-420) | |
| 41 | IPI00022434 | Same as KH28 | |
| | | gi: 113576 | |
| | | P02768.2 | |
| 42 | IPI00298497 | Same as KH36 | |
| | | gi: 399492 | |
| | | P02675.2 | |
| 43 | IPI00965713 | MKRMVSWSFH KLKTMKHLLL LLLCVFLVKS QGVNDNEEGF FSARGHRPLD KKREEAPSLR | 60 |
| | gi: 399492 | PAPPPISGGG YRARPAKAAA TQKKVERKAP DAGGCLHADP DLGVLCPTGC QLQEALLQQE | 120 |
| | P02675.2 | RPIRNSVDEL NNNVEAVSQT SSSSFQYMYL LKDLWQKRQK QVKDNENVVN EYSSELEKHQ | 180 |
| | | LYIDETVNSN IPTNLRVLRS ILENLRSKIQ KLESDVSAQM EYCRTPCTVS CNIPVVSGKE | 240 |
| | | CEEIIRKGGE TSEMYLIQPD SSVKPYRVYC DMNTENGGWT VIQNRQDGSV DFGRKWDPYK | 300 |
| | | QGFGNVATNT DGKNYCGLPG EYWLGNDKIS QLTRMGPTEL LIEMEDWKGD KVKAHYGGFT | 360 |
| | | VQNEANKYQI SVNKYRGTAG NALMDGASQL MGENRTMTIH NGMFFSTYDR DNDGWLTSDP | 420 |
| | | RKQCSKEDGG GWWYNRCHAA NPNGRYYWGG QYTWDMAKHG TDDGVVWMNW KGSWYSMRKM | 480 |
| | | SMKIRPFFPQ Q | 491 |
| 44 | IPI00645363 | NSLYLQMNSLRAEDTALYYCAK (Sequence 96-117) | |
| | gi: | GPSVFPLAPSSK (Sequence 147-158) | |
| | | TPEVTCVVVDVSHEDPEVK (Sequence 281-299) | |
| | | FNWYVDGVEVHNAK (Sequence 300-313) | |
| | | ALPAPIEK (Sequence 352-359) | |
| | | GQPREPQVYTLPPSRDELTKNQVSLTCLVKGFYPSDIAVEWESNGQPENNYK (Sequence 366-417) | |
| 45 | IPI00219713 | Same as KH30 | |
| | gi: 20178280 | | |
| | P02679 | | |
| 46 | IPI00022371 | MKALIAALLL ITLQYSCAVS PTDCSAVEPE AEKALDLINK RRRDGYLFQL LRIADAHLDR | 60 |
| | gi: 123523 | VENTTVYYLV LDVQESDCSV LSRKYWNDCE PPDSRRPSEI VIGQCKVIAT RHSHESQDLR | 120 |
| | P04196.1 | VIDFNCTTSS VSSALANTKD SPVLIDFFED TERYRKQANK ALEKYKEEND DFASFRVDRI | 180 |
| | | ERVARVRGGE GTGYFVDFSV RNCPRHHFPR HPNVFGFCRA DLFYDVEALD LESPKNLVIN | 240 |
| | | CEVFDPQEHE NINGVPPHLG HPFHWGGHER SSTTKPPFKP HGSRDHHHPH KPHEHGPPPP | 300 |
| | | PDERDHSHGP PLPQGPPPLL PMSCSSCQHA TFGTNGAQRH SHNNNSSDLH PHKHHSHEQH | 360 |
| | | PHGHHPHAHH PHEHDTHRQH PHGHHPHGHH PHGHHPHGHH PHGHHPHCHD FQDYGPCDPP | 420 |
| | | PHNQGHCCHG HGPPPGHLRR RGPGKGPRPF HCRQIGSVYR LPPLRKGEVL PLPEANFPSF | 480 |
| | | PLPHHKHPLK PDNQPFPQSV SESCPGKFKS GFPQVSMFFT HTFPK | 525 |
| 47 | IPI00022371 | Same as KH46 | |
| | | gi: 123523 | |
| | | P04196.1 | |
| 48 | IPI00022463 | MRLAVGALLV CAVLGLCLAV PDKTVRWCAV SEHEATKCQS FRDHMKSVIP SDGPSVACVK | 60 |
| | gi: 313104271 | KASYLDCIRA IAANEADAVT LDAGLVYDAY LAPNNLKPVV AEFYGSKEDP QTFYYAVAVV | 120 |
| | P02787.3 | KKDSGFQMNQ LRGKKSCHTG LGRSAGWNIP IGLLYCDLPE PRKPLEKAVA NFFSGSCAPC | 180 |
| | | ADGTDFPQLC QLCPGCGCST LNQYFGYSGA FKCLKDGAGD VAFVKHSTIF ENLANKADRD | 240 |
| | | QYELLCLDNT RKPVDEYKDC HLAQVPSHTV VARSMGGKED LIWELLNQAQ EHFGKDKSKE | 300 |
| | | FQLFSSPHGK DLLFKDSAHG FLKVPPRMDA KMYLGYEYVT AIRNLREGTC PEAPTDECKP | 360 |
| | | VKWCALSHHE RLKCDEWSVN SVGKIECVSA ETTEDCIAKI MNGEADAMSL DGGFVYIAGK | 420 |
| | | CGLVPVLAEN YNKSDNCEDT PEAGYFAIAV VKKSASDLTW DNLKGKKSCH TAVGRTAGWN | 480 |
| | | IPMGLLYNKI NHCRFDEFFS EGCAPGSKKD SSLCKLCMGS GLNLCEPNNK EGYYGYTGAF | 540 |
| | | RCLVEKGDVA FVKHQTVPQN TGGKNPDPWA KNLNEKDYEL LCLDGTRKPV EEYANCHLAR | 600 |
| | | APNHAVVTRK DKEACVHKIL RQQQHLFGSN VTDCSGNFCL FRSETKDLLF RDDTVCLAKL | 660 |
| | | HDRNTYEKYL GEEYVKAVGN LRKCSTSSLL EACTFRRP | 698 |
| 49 | IPI00023006 | MCDDEETTAL VCDNGSGLVK AGFAGDDAPR AVFPSIVGRP RHQGVMVGMG QKDSYVGDEA | 60 |
| | gi: 54036697 | QSKRGILTLK YPIEHGIITN WDDMEKIWHH TFYNELRVAP EEHPTLLTEA PLNPKANREK | 120 |
| | P68032.1 | MTQIMFETFN VPAMYVAIQA VLSLYASGRT TGIVLDSGDG VTHNVPIYEG YALPHAIMRL | 180 |
| | | DLAGRDLTDY LMKILTERGY SFVTTAEREI VRDIKEKLCY VALDFENEMA TAASSSSLEK | 240 |
| | | SYELPDGQVI TIGNERFRCP ETLFQPSFIG MESAGIHETT YNSIMKCDID IRKDLYANNV | 300 |
| | | LSGGTTMYPG IADRMQKEIT ALAPSTMKIK IIAPPERKYS VWIGGSILAS LSTFQQMWIS | 360 |
| | | KQEYDEAGPS IVHRKCF | 377 |
| 50 | IPI00021841 | Same as KH 37 | |
| | gi: 113992 | | |
| | P02647.1 | | |
| 51 | IPI00023006 | Same as KH49 | |
| | gi: 54036697 | | |
| | P68032.1 | | |
| 52 | IPI00930226 | MEEEIAALVI DNGSGMCKAG FAGDDAPRAV FPSIVGRPRH QGVMVGMGQK DSYVGDEAQS | 60 |
| | gi: 54036678 | KRGILTLKYP IEHGIVTNWD DMEKIWHHTF YNELRVAPEE HPVLLTEAPL NPKANREKMT | 120 |
| | P63261.1 | QIMFETFNTP AMYVAIQAVL SLYASGRTTG IVMDSGDGVT HTVPIYEGYA LPHAILRLDL | 180 |
| | | AGRDLTDYLM KILTERGYSF TTTAEREIVR DIKEKLCYVA LDFEQEMATA ASSSSLEKSY | 240 |
| | | ELPDGQVITI GNERFRCPEA LFQPSFLGME SCGIHETTFN SIMKCDVDIR KDLYANTVLS | 300 |
| | | GGTTMYPGIA DRMQKEITAL APSTMKIKII APPERKYSVW IGGSILASLS TFQQMWISKQ | 360 |
| | | EYDESGPSIV HRKCF | 375 |
| 53 | gi: 194373497 | MEESLPTNPD SSTMGPWCYT TDPTVRRQEC SIPVCGQDQV TVAMTPRSEG SSVNLSPPLE | 60 |
| | BAG56844.1 | QCVPDRGQQY QGRLAVTTHG LPCLAWASAQ AKALSKHQDF NSAVQLVENF CRNPDGDEEG | 120 |
| | | VWCYVAGKPG DFGYCDLNYC EEAVEEETGD GLDEDSDRAI EGRTATSEYQ TFFNPRTFGS | 180 |
| | | GEADCGLRPL FEKKSLEDKT ERELLESYID GRIVEGSDAE IGMSPWQVML FRKSPQELLC | 240 |
| | | GASLISDRWV LTAAHCLLYP PWDKNFTEND LLVRIGKHSR TRYERNIEKI SMLEKIYIHP | 300 |
| | | RYNWRENLDR DIALMKLKKP VAFSDYIHPV CLPDRETAAS LLQAGYKGRV TGWGNLKETW | 360 |
| | | TANVGKGQPS VLQVVNLPIV ERPVCKDSTR IRITDNMFCA GYKPDEGKRG DACEGDSGGP | 420 |
| | | FVMKSPFNNR WYQMGIVSWG EGCDRDGKYG FYTHVFRLKK WIQKVIDQFG E | 471 |
| 54 | gi: 194380034 | MNQLRGKKSC HTGLGRSAGW NIPIGLLYCD LPEPRKPLEK AVANFFSGSC APCADGTDFP | 60 |
| | BAG58369.1 | QLCQLCPGCG CSTLNQYFGY SGAFKCLKDG AGDVAFVKHS TIFENLANKA DRDQYELLCL | 120 |
| | | DNTRKPVDEY KDCHLAQVPS HTVVARSMGS KEDLIWELLN QAQEHFGKDK SKEFQLFSSP | 180 |
| | | HGKDLLFKDS AHGFLKVPPR MDAKMYLGYE YVTAIRNLRE GTCPEAPTDE CKPVKWCALS | 240 |
| | | HHERLKCDEW SVNSVGKIEC VSAETTEDCI AKIMNGEADA MSLDGGFVYI AGKCGLVPVL | 300 |
| | | AENYNKSDNC EDTPEAGYFA VAVVKKSASD LTWDNLKGKK SCHTAVGRTA GWNIPMGLLY | 360 |
| | | NKINHCRFDE FFSEGCAPGS KKDSSLCKLC MGSGLNLCEP NNKEGYYGYT GAFRCLVEKG | 420 |
| | | DVAFVKHQTV PQNTGGKNPD PWAKNLNEKD YELLCLDGTR KPVEEYANCH LARAPNHAVV | 480 |
| | | TRKDKEACVH KILRQQQHLF GSNVTDCSGN FCLFRSETKD LLFRDDTVCL AKLHDRNTYE | 540 |
| | | KYLGEEYVKA VGNLRKCSTS SLLEACTFRR P | 571 |
| 55 | gi: 194380034 | Same as 54 | |
| | BAG58369.1 | | |

By the present invention it has been shown that purified plasmas containing varying combinations and concentrations of KH proteins have vast implications for the treatment of a large host of diseases, viral infections, and other disorders. As described in more detail below, embodiments of the current invention involve purified plasma constructs, their newly discovered proteins, and their use in the treatment of: HIV 1 and 2; hepatitis B; hepatitis C; influenza; glucose uptake related disorders, e.g. diabetes; atherosclerosis and related cardiovascular diseases; high cholesterol levels; H1N1; arthritis; tumor progression; and parkinson's disease.

### Embodiments of the Invention

Study Title: In Vitro Anti-HIV Activity of Human Plasma Derived Proteins on HIV-RT Enzyme

I. Study Objective:

To Analyze Human Plasma Derived Proteins for Anti-HIV Activity on HIV-RT Enzyme

II. Study Protocols:

1. Materials:

1.1 Samples Information:

RAAS provided the test articles in the form of dry powder or liquid (Table 1). Wuxi provided reference compound in DMSO solution.

TABLE 1
Sample information

| Name | Protein conc. | Formulation | Diluents |
|---|---|---|---|
| AFOD KH | 10% | Liquid | |
| AFCC KH | 3.50% | Liquid | |
| AFCC RAAS 1 | 4% | Lyophilized | AFOD KH 10 mL |
| AFCC RAAS 4 | 0.0020% | Lyophilized | AFOD KH 10 mL |
| AFCC RDNA | 0.00001% | Lyophilized | AFOD KH 10 mL |

1.2 Reagents:

TABLE 2
List of reagents

| Reagents/Plates | Vendor | Cat.# |
|---|---|---|
| HIV-1 Reverse | Merck | 382129-500U |
| Transcriptase wild | | |
| type enzyme | | |
| Avidin standard plates | MSD | MSD-L15AA-6 |
| RNA template t500 | IBA GMBH | Cat. #89142N/S |
| synthetic piece of RNA | | |
| CHAPS | Pierce | Pierce-28300 |
| EGTA | Sigma | Sigma-E3889-10G |
| DTT | Sigma | Sigma-43815-5G |
| d-ATP | Sigma | Sigma-D6500-10MG |
| d-GTP | Sigma | D4010-10MG |
| d-CTP-Na 2 | Sigma | D4635-10MG |
| Water (DEPC treated) | Invitrogen | Invitrogen-750023 |
| dry biopD500 primer | Shanghai | |
| | Shenggong | |
| BSA | Sigma | Sigma-A3294 |
| 4x Read buffer T | MSD | MSD-R92TD-1 |
| Ru-d-UTP | MSD | Lot: DG2005245071 |
| 96-well round bottom | Costar | Costar-3365 |
| polypropylene plates | | |
| PCR tubes | AXYGEN | AXYGEN-PCR-0208-C |
| PCR tube covers | AXYGEN | AXYGEN-PCR-2CP-RT-C |

1.3 Instrument
Sector Imager S6000 (MesoScale Discovery MSD)
Epmotoin (Eppendorf)
Janus (perkinelmer)
Orbital shaker

2. Methods

2.1 IC50 Measurement

2.2.1 Drug Treatment:

Human plasma derived protein dilutions are made by using EpMotion with 2-fold serial dilutions for 10 concentrations, each in duplicate.
a) Add 30 μL of enzyme solution per well of the Costar 96 well plates.
b) Add 5 μL of test article or PBS or DMSO.
c) Seal plate and shake for 2 minutes on an orbital shaker
d) Incubate for 30 minutes on an orbital shaker at room temperature.
e) Add 15 μL of the Master Mix to initiate the reaction.
f) Seal plate and shake for 5-10 minutes.
g) Incubate at 37 degree for 90 minutes.
h) While this is incubating, add 100 pt of 5% BSA in PBS to the wells of the avidin plates.
i) Seal the avidin plates and incubate for 1 hour at room temperature.
j) After the 90 minute incubation, add 60 μL of quenching buffer to the reaction wells.
k) Seal the plates and incubate for 5 minutes on the plate shaker.
l) Transfer 50 μL of the well contents to MSD blocked plates (the blocking buffer is simply dumped off. No wash is needed).
m) Incubate MSD plates at RT for 60 minutes.
n) Freshly dilute the 4× read buffer T to 1× using distilled water (not DEPC-treated)
o) Wash MSD plates 3 times with 150 μL of PBS per well per wash.
p) Add 150 pt of 1× read buffer T to the wells.
q) Read on the Sector Imager Instrument.

2.2.2 Sample or Compound Addition

Test samples were diluted in PBS as 3.5×10⁴ μg/ml stocks. Sample dilutions are made by using Epmotion with 2-fold serial dilutions for 10 concentrations plus PBS (see below for final compound concentrations in the HIV-RT enzyme assay). Reference compound were dissolved in DMSO as 10 mM stocks and dilutions are made by using Epmotion with 3-fold serial dilutions for 10 concentrations plus DMSO (see below for final compound concentrations).

TABLE 3
Sample or compound concentrations for IC50 measurement

Name

| | Concentration (ug/ml) | | | | | | | | | |
|---|---|---|---|---|---|---|---|---|---|---|
| AFOD KH | 400 | 200 | 100 | 50 | 25 | 12.5 | 6.25 | 3.1 | 1.6 | 0.8 |
| AFCC KH | 400 | 200 | 100 | 50 | 25 | 12.5 | 6.25 | 3.1 | 1.6 | 0.8 |
| AFCC RAAS 1 | 400 | 200 | 100 | 50 | 25 | 12.5 | 6.25 | 3.1 | 1.6 | 0.8 |
| AFCC RAAS 4 | 400 | 200 | 100 | 50 | 25 | 12.5 | 6.25 | 3.1 | 1.6 | 0.8 |
| AFCC RDNA | 400 | 200 | 100 | 50 | 25 | 12.5 | 6.25 | 3.1 | 1.6 | 0.8 |
| | Concentration (nM) | | | | | | | | | |
| Reference | 100 | 33.3 | 11.1 | 3.7 | 1.2 | 0.4 | 0.1 | 0.05 | 0.02 | 0.01 |
| Compound | | | | | | | | | | |

2.2.3 Data Analysis:

Percent of HIV-RT inhibition by protein or compound is calculated using the following equation:

% Inh.=[1−(Signal of sample−Signal of control)/(Signal of DMSO or PBS control−Signal of control)]*100.

Dose-response curves are plotted using Prism

III. Assay Results:

3.1 Raw Data from the HIV-RT Enzyme Assay.

3.1.1 HIV-RT Enzyme Assay Plate Map*:

Plate 1

| column | column | column | column | column | column | column | column | column | column | column | column |
|---|---|---|---|---|---|---|---|---|---|---|---|
| 1 | 2 | 3 | 4 | 5 | 6 | 7 | 8 | 9 | 10 | 11 | 12 |

| | | | | |
|---|---|---|---|---|
| raw | A | PBS | AFOD KH | BG |
| raw | B | | | |
| raw | C | | AFCC KH | |
| raw | D | | | |
| raw | E | BG | AFCC RAAS 1 | PBS |
| raw | F | | | |
| raw | G | | Reference Compound | |
| raw | H | | | |

* BG: background

Plate 2

| column | column | column | column | column | column | column | column | column | column | column | column |
|---|---|---|---|---|---|---|---|---|---|---|---|
| 1 | 2 | 3 | 4 | 5 | 6 | 7 | 8 | 9 | 10 | 11 | 12 |

| | | | | |
|---|---|---|---|---|
| raw | A | PBS | AFCC RAAS 4 | BG |
| raw | B | | | |
| raw | C | | AFCC RDNA | |
| raw | D | | | |
| raw | E | BG | Reference Compound | PBS |
| raw | F | | | |
| raw | G | | DMSO | |
| raw | H | | | |

* BG: background

3.1.2 Raw Data

Plate 1:

| | | column | column | column | column | column | column | column | column | column | column | column | column |
|---|---|---|---|---|---|---|---|---|---|---|---|---|---|
| | | 1 | 2 | 3 | 4 | 5 | 6 | 7 | 8 | 9 | 10 | 11 | 12 |
| raw | A | 2439 | 1596 | 2113 | 2160 | 2304 | 2448 | 2214 | 2152 | 2307 | 2360 | 2357 | 60 |
| raw | B | 2569 | 1866 | 2154 | 2343 | 2351 | 2371 | 2397 | 2317 | 2310 | 2454 | 2245 | 64 |
| raw | C | 2571 | 281 | 329 | 393 | 563 | 805 | 1157 | 1683 | 2011 | 2304 | 2384 | 60 |
| raw | D | 2361 | 267 | 306 | 376 | 518 | 762 | 1156 | 1600 | 1912 | 2158 | 2185 | 58 |
| raw | E | 59 | 1238 | 1782 | 2097 | 2230 | 2299 | 2326 | 2374 | 2368 | 2329 | 2449 | 2267 |
| raw | F | 52 | 1248 | 1812 | 2166 | 2300 | 2406 | 2462 | 2398 | 2369 | 2346 | 2353 | 2366 |
| raw | G | 54 | 87 | 142 | 246 | 469 | 850 | 1241 | 1629 | 1791 | 1873 | 1851 | 2263 |
| raw | H | 53 | 85 | 132 | 241 | 474 | 833 | 1349 | 1651 | 1813 | 1924 | 1907 | 2438 |

Plate 2:

| | | column | column | column | column | column | column | column | column | column | column | column | column |
|---|---|---|---|---|---|---|---|---|---|---|---|---|---|
| | | 1 | 2 | 3 | 4 | 5 | 6 | 7 | 8 | 9 | 10 | 11 | 12 |
| raw | A | 2491 | 1713 | 1940 | 2168 | 2411 | 2358 | 2378 | 2459 | 2289 | 2262 | 2038 | 43 |
| raw | B | 2596 | 1674 | 2220 | 2344 | 2547 | 2491 | 2418 | 2541 | 2443 | 2476 | 2104 | 45 |
| raw | C | 2539 | 1747 | 2176 | 2381 | 2522 | 2388 | 2433 | 2314 | 2459 | 2358 | 2369 | 44 |
| raw | D | 2544 | 1689 | 2123 | 2305 | 2453 | 2385 | 2400 | 2426 | 2204 | 2049 | 2168 | 39 |
| raw | E | 44 | 91 | 146 | 270 | 514 | 957 | 1429 | 1801 | 1807 | 1895 | 1880 | 2142 |
| raw | F | 38 | 85 | 139 | 263 | 472 | 946 | 1377 | 1614 | 1708 | 1850 | 1853 | 2292 |
| raw | G | 45 | 2119 | 2160 | 2084 | 2046 | 2069 | 1963 | 1975 | 2002 | 1961 | 1912 | 2343 |
| raw | H | 43 | 2052 | 2038 | 2039 | 1975 | 1954 | 1860 | 1968 | 1972 | 1875 | 2042 | 2405 |

3.2 Activity of the Samples or Compounds.

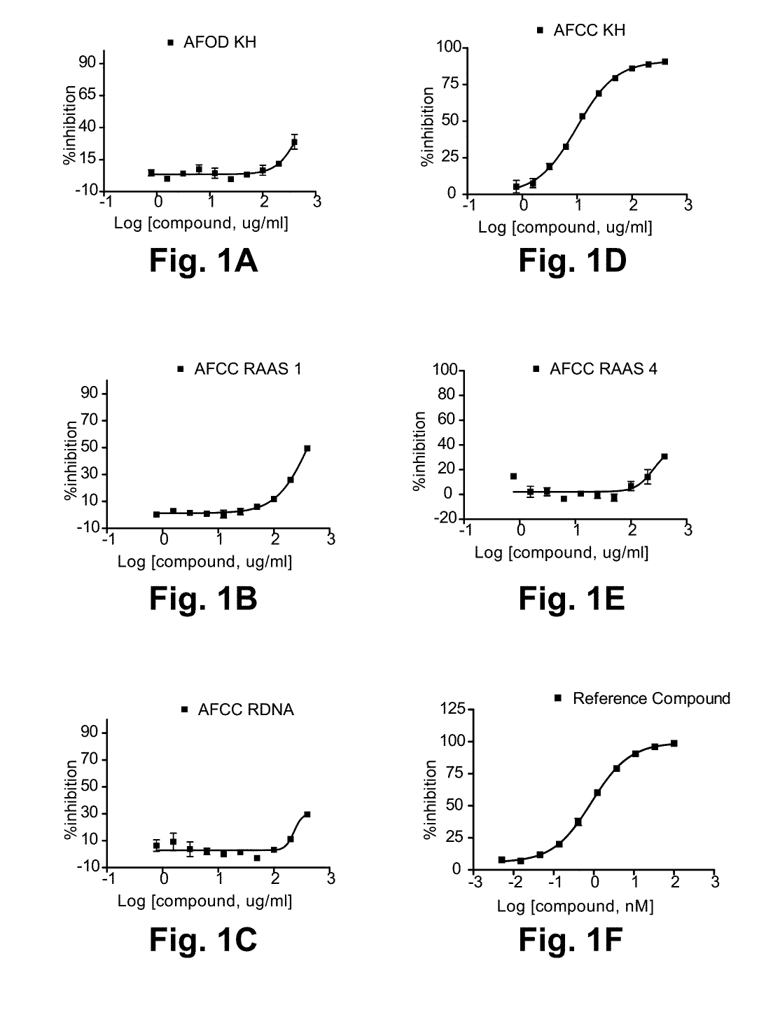

IC50 values are summarized in Table 4. GraphPad Prism files containing dose-dependent curves are presented in this report, as shown in FIG. 1.

4. Conclusions

The Z factors of the two plate were 0.84 (plate 1), 0.80 (plate 2), which were much better than QC standard of 0.5. Therefore, the assay data met our QC qualification.
The IC50s of positive control in this study were 0.9 nM (plate 1), 1.2 nM (plate 2) and these results are consistent with our previous data.

The Results of Neutralization of HIV-1 Env-Pseudotyped Virus

Samples and Control
1. Test samples: 3 in total, AFCC KH, AFCC RAAS and AFOD RAAS110 respectively
2. Positive control compound: 3 in total, AMD 3100 (inhibitor of CXCR4), nifeviroc (inhibitor of CCR5) and Ibalizumab (anti-CD4 monoclonal antibody). All three drugs can inhibit the entry of virus into cells.
   AMD 3100 was initially developed at the Johnson Matthey Technology Centre for potential use in the treatment of HIV because of its role in the blocking of CXCR4, a chemokine receptor which acts as a co-receptor for certain strains of HIV (along with the virus's main cellular receptor, CD4).
   NIFEVIROC, a small molecular compound, is a proprietary drug candidate which is developed by TARGETDRUG and holds great promise in inhibiting HIV-1 replication in infected patients. Preclinical studies suggested that NIFEVIROC is a specific CCR5 antagonist determined by multiple receptor functional assays.
   Ibalizumab (TMB-355 previously known as TNX-355) is a non-immunosuppressive monoclonal antibody that binds CD4, the primary receptor for HIV, and inhibits the viral entry process.
3. Tested virus: 10 strains, they are
   (1) B′ subtype virus: CNE6 and CNE11;
      BC recombinant subtype virus: CNE15 and CNE30;
      CRF01_AE recombinant subtype virus: CNE5 and CNE55;
      The standard HIV-1 strain virus: sf162, HXB2 and JRFL,
      All above HIV-1 virus are CCR5 receptor affinity except HXB2 is CXCR4 receptor affinity.
   (2) Control virus: AMLV.

Test Method
1. Test samples were diluted at 1:20 as start and then 1:60, 1:180, 1:540, 1:1620, 1:4860, 1:14580, 1:43740. It was 3-fold dilution and 8 dilutions in total.
2. Positive control drug was started to dilute from:
   AMD3100: 10 uM
   Nifeviroc: 0.05 uM
   Ibalizumab: 10 ug/ml
   It was 3-fold dilution and 8 dilutions in total.

Results
1. AFCC KH: no activity in preventing the entry of virus into cells. But it enhances virus invade into cells.
2. AFOD RAAS 110 (AT III): no activity in preventing the entry of virus into cells. But it strongly helps virus invade into cells.
3. AFCC RAAS (15% PCC): it showed 50% inhibition of virus in five strains of virus, which are CNE15, CNE30, CNE55, HXB2 and JRFL, with dose dependent response. It showed weak inhibition in two strains (CNE5 and AMLV) and no effects on the rest 3 strains (CNE6, CNE 11 and sf162)

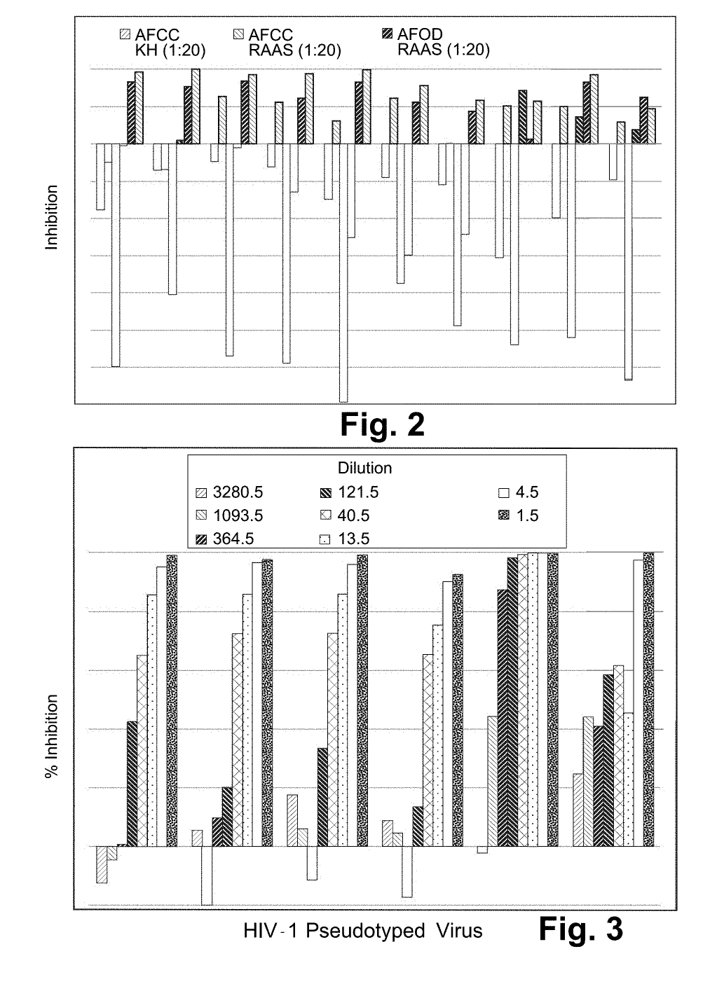

FIG. 2

The Supplementary Results of Neutralization of HIV-1 Env-Pseudotyped Virus

Samples and Control
4. Test samples: AFCC RAAS
5. Tested virus: 5 strains, they are
   (3) BC recombinant subtype virus: CNE15 and CNE30;
      CRF01_AE recombinant subtype virus: CNE55;
      The standard HIV-1 strain virus: HXB2 and JRFL,
      All above HIV-1 virus are CCR5 receptor affinity except HXB2 is CXCR4 receptor affinity.
   (4) Control virus: AMLV.

Test Method
3. Test samples were diluted at 1:1.5 as start and then 1:4.5, 1:13.5, 1:40.5, 1:121.5, 1:364.5, 1:1093.5, and 1:3280.5. It was 3-fold dilution and 8 dilutions in total.

Results
4. It has been shown in FIG. 1 that the inhibition rate of AFCC RAAS in 5 HIV-1 strains and control virus AMLV. The conclusion is that the inhibition rate is about 60% when the dilution was less than 1:40 and the inhibition also was observed in control virus AMLV. Cell toxicity was found in high concentrations via observing cell morphology 48 hours after treatment. Thus cell toxicity test was then conducted.

FIG. 3
4. Cell toxicity test: in this study, we tested the toxicity of AFCCKH, AFOD RAAS 101 and AFCC RAAS. Test samples were diluted at 1:1.5 as start and then 1:4.5, 1:13.5, 1:40.5, 1:121.5, 1:364.5, 1:1093.5, and 1:3280.5. It was 3-fold dilution and 8 dilutions in total. The test kit is cell counting kit 8 (CCK-8). The procedure is according to manufacturer's manual. It has been shown that there is some cell toxicity of RAAS. The inhibition of HIV virus probably is caused by cell toxicity.

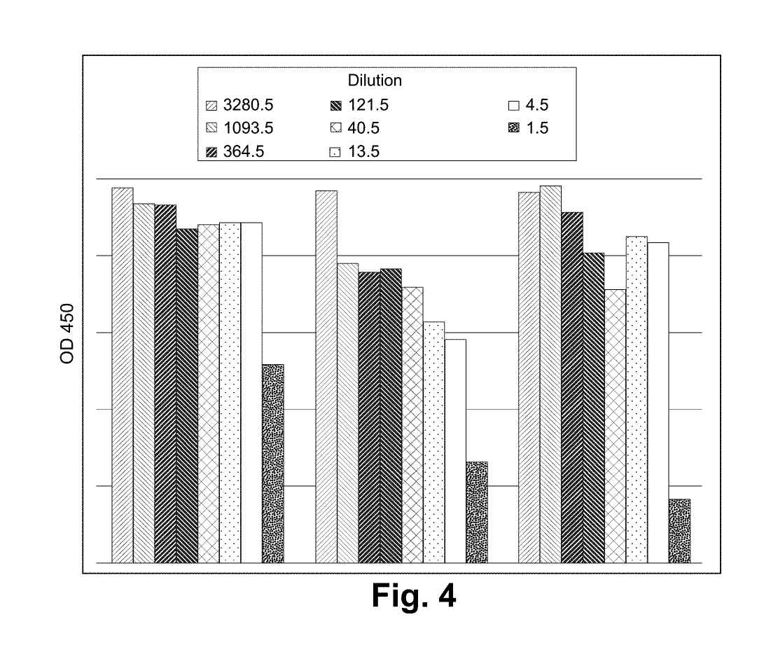

FIG. 4

Suggestion for Further Study

To decrease the toxicyte to cell, and ensure the high inhibition of virus at high protein concentration.
1. further increase the protein concentration.
2. Use cell culture medium (DMEM+10% FBS) as the diluent of products when preparing the samples.

Study Title: Test Human Plasma Derived Proteins Against HCV Genotype 1a, 1b and 2a Replicons for Antiviral Activity (EC₅₀)

I. Study Objective

To Analyze Human Plasma Derived Proteins for Anti-HCV Activity (EC₅₀) and Cytotoxicity (CC₅₀) Using HCV 1a, 1b and 2a Replicon Culture Systems

II. Study Protocols

3. Materials:

1.1 Cell Line:

Replicon cell lines 1a and 2a were established following published methods (1,2) using Huh7 by G418 selection. The replicons were assembled using synthetic gene fragments. The GT 1a line is derived from H77 and contains PVIRES-Luciferase-Ubi-Neo, and two adaptive mutations: P1496L, S22041. The 2a line contains no adaptive mutations and encodes a Luciferase reporter. The 1b replicon plasmid is also assembled using synthetic gene fragments. The replicon genome contains PVIRES-Luciferase Ubi-Neo gene segments and harbors 1 adaptive mutation (S22041), and the backbone is Con1.

1.2 Compounds:

The test articles are supplied in the form of dry powder or 10 mM solution, and Ribavirin as control, in duplicate.

1.3 Reagents:

TABLE 1
List of reagents

| Reagent | Vendor | Catalog Number |
|---|---|---|
| Dimethyl sulfoxide (DMSO) | Sigma | Cat#34869 |
| DMEM | Invitrogen | Cat#11960-044 |
| Fetal Bovine Serum (FBS) | Gibco | Cat#16140 |
| Penicillin-Streptomycin | Invitrogen | Cat#15070063 |
| MEM non-essential amino acids | Invitrogen | cat#11140-050 |
| L-Glutamine | Invitrogen | Cat#25030-081 |
| Trypsin/EDTA | Invitrogen | Cat#25200-072 |
| DPBS/Modified | Hyclone | SH30028.01B |
| 96 well cell plate | Greiner | Cat#655090 |
| CellTiter fluor | Promega | Cat#G6082 |
| Bright-Glo | Promega | Cat#E2650 |

1.4 Instrument
Envision (Perkinelmer)
Multidrop (Thermo)
Janus (Perkinelmer)

4. Methods

2.1 Cell Addition

T150 flask containing 1a, 1b and 2a replicons cell monolayer is rinsed with 10 ml pre-warmed PBS. Add 3 ml of pre-warmed Trypsin 0.25% and incubate at 5% CO₂, 37□ for 3 minutes. Nine milliliters of DMEM complete media are added, and the cells are blown for 30s by pipetting. The cells are counted using hemocytometer.

1a, 1b and 2a replicons cells are resuspended in medium containing 10% FBS to reach a cell density of 64,000 cells/ml (to obtain a final cell plating density of 8000 cells/125 ul/well). Plate cells in Greiner 96 black plate using Multidrop. Incubate plate at 5% CO₂, 37□ for 4 hours.

2.2 Compound Addition

RAAS provided the test articles in the form of dry powder or liquid (Table 2). Test samples were diluted in PBS as 3.5×10⁴ μg/ml stocks. Sample dilutions are made by Janus with 2-fold serial dilutions for 10 concentrations plus PBS. Ribavirin is also diluted by Janus with 2-fold for 10 concentrations. The final sample concentrations of the HCV replicon assay are described in Table 3.

TABLE 2
Sample information

| Name | Protein conc. | Formulation | Diluents |
|---|---|---|---|
| AFOD KH | 10% | Liquid | |
| AFCC KH | 3.50% | Liquid | |
| AFCC RAAS 1 | 4% | Lyophilized | AFOD KH 10 mL |
| AFCC RAAS 4 | 0.0020% | Lyophilized | AFOD KH 10 mL |
| AFCC RDNA | 0.00001% | Lyophilized | AFOD KH 10 mL |

TABLE 3
Sample or compound concentrations for EC₅₀ and CC₅₀ measurement

| Name | HCV Genotype | | | | | | | | | | |
|---|---|---|---|---|---|---|---|---|---|---|---|
| | | Concentration (μg/ml) | | | | | | | | | |
| AFOD KH | 1a/1b/2a | 400 | 200 | 100 | 50 | 25 | 12.5 | 6.3 | 3.1 | 1.6 | 0.8 |
| AFCC KH | | 400 | 200 | 100 | 50 | 25 | 12.5 | 6.3 | 3.1 | 1.6 | 0.8 |
| AFCC RAAS 1 | | 400 | 200 | 100 | 50 | 25 | 12.5 | 6.3 | 3.1 | 1.6 | 0.8 |
| AFCC RAAS 4 | | 400 | 200 | 100 | 50 | 25 | 12.5 | 6.3 | 3.1 | 1.6 | 0.8 |
| AFCC RDNA | | 400 | 200 | 100 | 50 | 25 | 12.5 | 6.3 | 3.1 | 1.6 | 0.8 |
| | | Concentration (μM) | | | | | | | | | |
| Ribavirin | | 320 | 160 | 80 | 40 | 20 | 10 | 5 | 2.5 | 1.3 | 0.6 |

2.3 Detection (after 72 Hours of Incubation)

Bright-Glo Luiferase and CellTiter-Fluor™ are prepared and stored in dark while allowing to equilibrate to room temperature. Plates are removed from incubator to allow equilibration to room temperature. Multidrop is used to add 40 ul CellTiter-Fluor™ to each well of compound-treated cells. The plates are incubated for 0.5 hour, and then read on an Envision reader for cytotoxicity calculation. The cytotoxicity is calculates using the equation below.

$$\% Cytotoxicity = ( 1 - \frac{Cmpd - Background}{DMSO - Background} ) \times 100$$

100 ul of Bright-Glo are added to each well, incubated for 2 minutes at room temperature, and chemi-luminescence (an indicator of HCV replication) is measured for EC₅₀ calculation.

The anti-replicon activity (% inhibition) is calculated using the equation below

$$\% Inhibition = ( 1 - \frac{Cmpd - background}{DMSO - background} ) \times 100$$

Dose-response curves are plotted using Prism.

III. Assay Results

1 Assay Plate Map

2 Raw Data

2.1 Raw Data of Cytotoxicity Assay

2.2 Raw Data of Anti-Replicon Activity Assay

3 Cytotoxicity and Anti-Replicon Activity of the Human Plasma Derived Proteins.

CC₅₀ and EC50 values are summarized in Table 4. GraphPad Prism files containing dose-dependent curves are presented in this report. CC₅₀ and EC₅₀ values are shown in FIG. 1 and FIG. 2 respectively.

IV. Conclusions
The Z factors of the cytotoxicity assay plates are 0.83 (1a-plate1), 0.79 (1a-plate2), 0.71 (1b-plate1), 0.68 (1b-plate2), 0.65 (2a-plate1) and 0.83 (2a-plate2), which are better than our QC standard.
The Z factors of the anti-replicon assay plates are 0.75 (1a-plate1), 0.70 (1a-plate2), 0.87 (1b-plate1), 0.75 (1b-plate2), 0.58 (2a-plate1) and 0.75 (2a-plate2), which are better than our QC standard.
EC₅₀ of the positive control Ribavirin in this study are 57.58 uM (1a), 39.04 uM (1b), and 37.44 (2a), which are consistent with our previous data.

V. References
1. Mutations in Hepatitis C Virus RNAs Conferring Cell Culture Adaptation V. Lohmann et al., 2001 J. Virol.
2. Development of a replicon-based phenotypic assay for assessing the drug susceptibilities of HCV NS3 protease genes from clinical isolates. Qi X et al., Antiviral Res. 2009 February; 81(2:)166-73

In Vitro Anti-HBV Efficacy Test

Method and Materials

1) Cell model: HepG2 cell infected with HBV virus, which is HepG2 2.2.15 cell

2) Cell viability is analyzed by MTT method

3) EIA test to detect the inhibition of HBsAg and HBeAg

4) Positive control drug: Lamivudine

5) RT-PCR detection of HBV-DNA

Procedure

1) Toxicity of Drug to Cell

HepG2 2.2.15 cells are seeded in 96-well plate. Fresh medium with various concentration of drug is added 48 hour later. Cell viability is analyzed 9 days later by MTT method.

2) The Inhibition of HBV Virus

HepG2 2.2.15 cells are seeded in 96-well plate. Fresh medium with various concentration of drug is added 48 hour later. The HBsAg and HBeAg are detected 5 days, 7 days, and 10 days later. RT-PCR detection of HBV-DNA

Results

| AFOD | HBsAg | | HBeAg | |
|---|---|---|---|---|
| (μg/mL) | OD | Inhibition rate % | OD | Inhibition rate % |
| 10 | 0.611 | 47.6 | 1.020 | 17.6 |
| 5 | 0.695 | 40.4 | 1.059 | 14.5 |
| 2.5 | 0.775 | 33.5 | 1.115 | 10.0 |
| 1.25 | 0.897 | 23.1 | 1.165 | 5.9 |
| Negative control | 1.166 | / | 1.238 | / |

Study Title: In Vitro Test of Human Plasma Derived Proteins Against Influenza for Antiviral Activity (EC50)

Influenza Study

I. Study Objective

To Test 2 Compounds from RAAS for Anti-Influenza Activity Against Strains A/Weiss/43 H1N1 in Cell Culture

II. Study Protocols:

3. Materials:

Cell Line:

MDCK cells

1.2 Compounds:

The test articles are supplied in the form of dry powder or 10 mM solution, and Oseltamivir as control, in duplicate.

1.3 Reagents:

The following table designations, such as Table 5.1, refer to tables of a first group of tables in the present application. Other groups of tables in the present application, which will be referred to later in the application, will contain some tables that have the same designations as tables of the first group.

TABLE 5.1
List of reagents and consumable

| Reagent | Vendor | Catalog Number |
|---|---|---|
| Dimethyl sulfoxide (DMSO) | Sigma | Cat#D8418 |
| SFM | Invitrogen | Cat# 12309-019 |
| Fetal Bovine Serum (FBS) | Gibco | Cat#16140 |
| Penicillin-Streptomycin | Invitrogen | Cat# 15140-122 |
| MEM non-essential amino | Invitrogen | cat# 11140-076 |
| acids | | |
| GlutaMAX-I Supplement | Invitrogen | Cat# 35050-061 |
| Trypsin/EDTA | Invitrogen | Cat# 25300-062 |
| PBS | Invitrogen | Cat#10010-049 |
| DPBS/Modified | Hyclone | SH30028.01B |
| 96 well cell plate | Corning | Cat#3599 |
| MTT | sigma | Cat# M2128 |

1.4 Instrument
speterphotemeter (Molecular Devices)
Multidrop (Thermo)
Janus (perkinelmer)

4. Methods

2.1 Cell Addition

T150 flask containing MDCK cell monolayer is rinsed with 10 ml pre-warmed PBS. Add 3 ml of pre-warmed Trypsin 0.25% and incubate at 5% CO₂, 37□ for 3 minutes. Nine milliliters of DMEM complete media are added, and the cells are blown for 30s by pipetting. The cells are counted using hemocytometer. MDCK cells are resuspended in SFM medium to reach a cell density of 50,000 cells/ml (to obtain a final cell plating density of 5000 cells/100 ul/well). Plate cells in 96 well plate using Multidrop. Incubate plate at 5% CO₂, 37□ for overnight.

2.2 Compound Addition

RAAS provided the test articles in the form of dry powder or liquid (Table 5.2). Test samples were diluted in PBS as 3.5×10⁴ μg/ml stocks. Sample dilutions are made by Janus with 2-fold serial dilutions for 8 concentrations plus PBS. Osletamivir is diluted with 3-fold for 8 concentrations. The final sample concentrations of the anti-influenza assay are described in Table 5.3.

TABLE 5.2
Sample information

| Name | Protein conc. | Formulation | Diluents |
|---|---|---|---|
| AFOD KH | 10% | Liquid | |
| AFCC KH | 3.50% | Liquid | |
| AFCC RAAS 1 | 4% | Lyophilized | AFOD KH 10 mL |
| AFCC RAAS 4 | 0.0020% | Lyophilized | AFOD KH 10 mL |
| AFCC RDNA | 0.00001% | Lyophilized | AFOD KH 10 mL |

TABLE 5.3
Sample or compound concentrations for EC₅₀ and CC₅₀ measurement

| Name | Concentration (μg/ml) | | | | | | | |
|---|---|---|---|---|---|---|---|---|
| AFOD KH | 400 | 200 | 100 | 50 | 25 | 12.5 | 6.3 | 3.1 |
| AFCC KH | 400 | 200 | 100 | 50 | 25 | 12.5 | 6.3 | 3.1 |
| AFCC RAAS 1 | 400 | 200 | 100 | 50 | 25 | 12.5 | 6.3 | 3.1 |
| AFCC RAAS 4 | 400 | 200 | 100 | 50 | 25 | 12.5 | 6.3 | 3.1 |
| AFCC RDNA | 400 | 200 | 100 | 50 | 25 | 12.5 | 6.3 | 3.1 |
| Osletamivir | Concentration (μM) | | | | | | | |
| | 100.00 | 33.33 | 11.11 | 3.70 | 1.23 | 0.41 | 0.14 | 0.05 |

2.3 Detection (after 72 Hours of Incubation)

MTT solution is prepared freshly. Plates are removed from incubator to allow equilibration to room temperature. Multidrop is used to add 20 ul MTT to each well of compound-treated cells. The plates are incubated for 4 hour, and then read on a speterphotemeter for EC50 and cytotoxicity calculation.

The anti-influenza activity (% inhibition) is calculated using the equation below

$$\% Inhibition = ( 1 - \frac{Cmpd - background}{DMSO - background} ) \times 100$$

The cytotoxicity is calculates using the equation below:

% livability=(Cmpd/PBS control)*100

Dose-response curves are plotted using Prism.

III. Assay Results:

1 Assay Plate Map

2 Raw data

2.1 Raw Data of Anti-Influenza Assay

| | 1 | 2 | 3 | 4 | 5 | 6 | 7 | 8 | 9 | 10 | 11 | 12 |
|---|---|---|---|---|---|---|---|---|---|---|---|---|
| plate 1 | | | | | | | | | | | | |
| A | | | | | | | | | | | | |
| B | | 0.93 | 1.47 | 1.43 | 0.24 | 0.22 | 0.21 | 0.18 | 0.19 | 0.136 | 1.504 | |
| C | | 1.032 | 1.345 | 1.276 | 0.455 | 0.241 | 0.226 | 0.203 | 0.188 | 0.216 | 1.439 | |
| D | | 1.348 | 1.308 | 1.375 | 1.485 | 0.221 | 0.171 | 0.197 | 0.158 | 0.159 | 1.506 | |
| E | | 1.362 | 1.429 | 1.466 | 1.386 | 0.234 | 0.159 | 0.173 | 0.208 | 0.167 | 1.565 | |
| F | | 1.486 | 1.318 | 0.963 | 0.264 | 0.173 | 0.173 | 0.185 | 0.181 | 0.163 | 1.477 | |
| G | | 1.584 | 1.432 | 0.948 | 0.322 | 0.224 | 0.217 | 0.205 | 0.149 | 0.131 | 1.468 | |
| H | | | | | | | | | | | | |
| plate 2 | | | | | | | | | | | | |
| A | | | | | | | | | | | | |
| B | | 1.48 | 1.39 | 0.81 | 0.27 | 0.22 | 0.18 | 0.14 | 0.17 | 0.180 | 1.279 | |
| C | | 1.464 | 1.294 | 0.668 | 0.236 | 0.174 | 0.224 | 0.176 | 0.179 | 0.189 | 1.261 | |
| D | | 1.411 | 1.238 | 0.279 | 0.183 | 0.207 | 0.237 | 0.175 | 0.177 | 0.150 | 1.262 | |
| E | | 1.418 | 1.128 | 0.306 | 0.211 | 0.180 | 0.178 | 0.231 | 0.176 | 0.172 | 1.238 | |
| F | | 1.290 | 1.382 | 1.296 | 1.266 | 0.969 | 0.563 | 0.544 | 0.386 | 0.353 | 1.319 | |
| G | | 1.292 | 1.218 | 1.210 | 1.295 | 0.962 | 0.627 | 0.431 | 0.388 | 0.394 | 1.397 | |
| H | | | | | | | | | | | | |

Raw Data of Cytotoxicity Assay

| | 1 | 2 | 3 | 4 | 5 | 6 | 7 | 8 | 9 | 10 | 11 | 12 |
|---|---|---|---|---|---|---|---|---|---|---|---|---|
| plate 1 | | | | | | | | | | | | |
| A | | | | | | | | | | | | |
| B | | 1.49 | 1.61 | 1.58 | 1.42 | 1.03 | 1.18 | 1.13 | 1.10 | 1.161 | 1.209 | |
| C | | 1.593 | 1.550 | 1.482 | 1.440 | 0.995 | 1.173 | 1.337 | 1.043 | 1.122 | 1.261 | |
| D | | 1.366 | 1.332 | 1.230 | 1.301 | 1.321 | 1.279 | 1.227 | 1.322 | 1.238 | 1.306 | |
| E | | 1.308 | 1.323 | 1.225 | 1.273 | 1.268 | 1.247 | 1.274 | 1.357 | 1.318 | 1.326 | |
| F | | 1.788 | 1.718 | 1.471 | 1.418 | 1.406 | 1.373 | 1.295 | 1.340 | 1.257 | 1.270 | |
| G | | 1.798 | 1.741 | 1.455 | 1.543 | 1.471 | 1.320 | 1.352 | 1.367 | 1.275 | 1.216 | |
| H | | | | | | | | | | | | |
| plate 2 | | | | | | | | | | | | |
| A | | | | | | | | | | | | |
| B | | 1.793 | 1.799 | 1.852 | 1.776 | 1.796 | 1.639 | 1.626 | 1.650 | 1.626 | 1.524 | |
| C | | 1.842 | 1.870 | 1.818 | 1.939 | 1.773 | 1.690 | 1.631 | 1.649 | 1.675 | 1.564 | |
| D | | 1.822 | 1.897 | 1.849 | 1.891 | 1.688 | 1.689 | 1.641 | 1.637 | 1.713 | 1.617 | |
| E | | 1.830 | 1.944 | 1.913 | 1.874 | 1.812 | 1.606 | 1.630 | 1.652 | 1.605 | 1.570 | |
| H | | | | | | | | | | | | |

3 Cytotoxicity and Anti-Influenza Activity of the Human Plasma Derived Proteins.

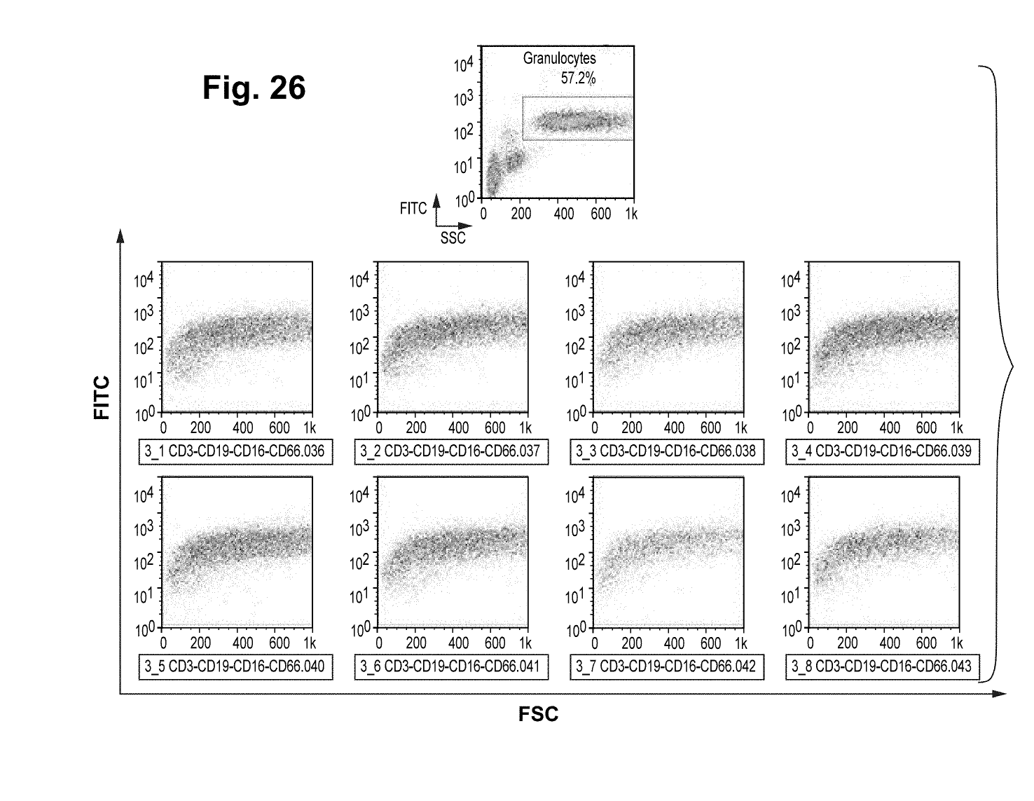

CC₅₀ and EC₅₀ values are summarized in Table 5.4. GraphPad Prism files containing dose-dependent curves are presented in this report. CC₅₀ and EC₅₀ values are shown in FIG. 26.17 and FIG. 26.21 respectively.

TABLE 5.4
CC₅₀ and EC₅₀ Summary of the human plasma derived proteins

| cpds | anti H1N1 EC50s (ug/ml) | CC50s (ug/ml) |
|---|---|---|
| AFOD KH | 69.06 | >400 |
| AFCC KH | 35.37 | >400 |
| AFCC RAAS 1 | 89.63 | >400 |
| AFCC RAAS 4 | 108.40 | >400 |
| AFCC RDNA | 154.90 | >400 |
| cpds | anti H1N1 EC50s (uM) | |
| Oseltamivir | 0.89 | |

IV. Conclusions
The EC₅₀ of the positive control Osletamivir in this study is 0.89 uM, which is consistent with our previous data.
The human plasma derived proteins showed anti-influenza activity in this study.

Characterization of Cultured Cells for RAAS

Executive Summary

This study is to analyze the cells in culture by flow cytometric analysis. The samples were provided by the client. First, all the samples were counted individually with Vi-CELL Cell Viability Analyzer (Beckman Coulter) for cell number and viability. Then the samples were stained with cellular markers for different lineages including T cells, B cells, granulocytes, natural killer (NK) cells. Normal human peripheral blood sample was used as controls for the staining

Among 59 samples, 30 samples contained cells. Only 10 samples had total cell number above 1×10⁵ and only 5 samples reached viability above 90%. In comparison with forward scatter (FSC)/side scatter (SSC) of distinct subpopulations of human peripheral blood cells, such as lymphocytes, granulocytes, monocytes and macrophages, unknown samples didn't obtain the same distribution shown by FACS Staining and distribution pattern of unknown samples also demonstrated they were not granulocytes, lymphocytes, or NK cells.

List of Abbreviations

| | |
|---|---|
| FACS | Flow Cytometry |
| BSA | Bovine serum albumin |
| FSC | Forward scatter |
| SSC | side scatter |
| NK cells | Natural killer cells |

Materials and Methods

Materials

Reagents

FITC, Anti-Human CD66, BD, Cat: 551479

FITC, Anti-Human CD34, BD, Cat: 560942

PE, Anti-Human CD3, BD, Cat: 561803

PE, Anti-Human CD146, BD, Cat: 561013

PE, Anti-Human CD56, BD, Cat: 561903

PE, Anti-Human CD14, BD, Cat: 561707

PE, Anti-Human CD11c, BD, Cat: 560999

PerCP-Cy5.5, Anti-Human CD16, BD, Cat: 560717

APC, Anti-Human CD19, BD, Cat: 561742

PE, Anti-Human CD41a, BD, Cat: 560979

ACK Lysis buffer, Invitrogen, Cat: A10492-01

PBS, Dycent Biotech (Shanghai) CO., Ltd. Cat: BJ141. FBS, Invitrogen Gibco, Cat: 10099141

BSA, Beyotime, ST023

Materials

Cell strainer (70 μm), BD, Cat: 352350

BD Falcon tubes (12×75 mm, 5 ml), BD, Cat: 352054

Equipment

Vi-CELL Cell Viability Analyzer, Beckman Coulter, Cat: 731050

FACSCalibur flow cytometer, BD, Cat: TY1218

Methods

Staining
Cells were placed into the 96-well (6×10⁵ cells/well) plate and blocked with 0.08% NaN3/PBS containing 1% FBS, 1% mouse serum and 2% BSA for 15 min at 4° C.
Cells were washed once with 1×PBS and resuspended with staining buffer (0.08% NaN3/PBS+1% FBS) with indicated antibodies for 30 min@ 4° C.
Cells were washed twice with 0.08% NaN3/PBS (200 μl per well) and resuspended with 400 μl 0.08% NaN3/PBS.
Excessive chunk from cell suspension were removed by filtrating through cell strainer. Cells were collected in BD Falcon tubes (12×75 mm, 5 ml) and analyzed by FACSCalibur.

Data Analysis

FACS data were analyzed by flowjo software.

Study Summary

Study Initiation Date and Completion Date

Cell samples were received on Apr. 26, 2012 and analyzed on Apr. 27.

Study Purpose

The purpose of this study was to characterize the unknown cells.

Study Results

Cell Count

59 cell samples were counted individually using Vi-CELL Cell Viability Analyzer (Beckman Coulter). The detailed information was listed in Table 1.

TABLE 1
Cell counting

| | | | | | | | Viability |
|---|---|---|---|---|---|---|---|
| Sample | Denisity ×10⁶/ml | Total cells | Viability | Sample | Denisity ×10⁶/ml | Total cells | (%) |
| ID | 0.00E+00 | 0.00E+00 | (%) | ID | 3.60E+04 | 3.60E+04 | 50 |
| | | | | 3_7 | 2.40E+04 | 2.40E+04 | 20 |
| 1_2 | 0.00E+00 | | 0.00E+00 | 3_8 | 2.40E+04 | 2.40E+04 | 40 |
| 1_3 | 0.00E+00 | | 0.00E+00 | 3_9 | 3.60E+04 | 3.60E+04 | 100 |
| 1_4 | 0.00E+00 | | 0.00E+00 | 3_10 | 3.60E+04 | 3.60E+04 | 60 |
| 1_5 | 0.00E+00 | | 0.00E+00 | 3_11 | 9.50E+04 | 9.50E+04 | 57.1 |
| 1_6 | 0.00E+00 | | 0.00E+00 | 3_12 | 2.40E+04 | 2.40E+04 | 40 |
| 1_7 | 0.00E+00 | | 0.00E+00 | 4_1 | 9.50E+04 | 9.50E+04 | 32 |
| 1_8 | 0.00E+00 | | 0.00E+00 | 4_2 | 3.80E+05 | 3.80E+05 | 69.6 |
| 1_9 | 0.00E+00 | | 0.00E+00 | 4_3 | 3.30E+05 | 3.30E+05 | 93.3 |
| 1_10 | 0.00E+00 | | 0.00E+00 | 4_4 | 1.20E+05 | 1.20E+05 | 35.7 |
| 1_11 | 0.00E+00 | | 0.00E+00 | 4_5 | 3.70E+05 | 3.70E+05 | 72.1 |
| 1_12 | 0.00E+00 | | 0.00E+00 | 4_6 | 2.50E+05 | 2.50E+05 | 87.5 |
| 2_1 | 4.80E+04 | | 4.80E+04 | | | | |
| 66.7 | | | | 4_7 | 1.80E+05 | 1.80E+05 | 37.5 |
| 2_2 | 0.00E+00 | | 0.00E+00 | 4_8 | 2.40E+05 | 2.40E+05 | 44.4 |
| 2_3 | 0.00E+00 | | 0.00E+00 | 4_9 | 3.30E+05 | 3.30E+05 | 96.6 |
| 2_4 | 0.00E+00 | | 0.00E+00 | 5_1 | 1.80E+05 | 1.80E+05 | 48.4 |
| 2_5 | 0.00E+00 | | 0.00E+00 | 5_2 | 2.40E+05 | 2.40E+05 | 55.6 |
| 2_6 | 0.00E+00 | | 0.00E+00 | 5_3 | 3.00E+05 | 3.00E+05 | 92.6 |
| 2_7 | 0.00E+00 | | 0.00E+00 | 5_4 | 2.70E+05 | 2.70E+05 | 79.3 |
| 2_8 | 0.00E+00 | | 0.00E+00 | 5_5 | 2.10E+05 | 2.10E+05 | 51.4 |
| 2_9 | 0.00E+00 | | 0.00E+00 | 5_6 | 2.40E+04 | 2.40E+04 | 66.7 |
| 2_10 | 0.00E+00 | | 0.00E+00 | 6_1 | 1.20E+04 | 1.20E+04 | 50 |
| | | | | 6_2 | 1.20E+04 | 1.20E+04 | 50 |
| 2_11 | 0.00E+00 | | 0.00E+00 | 6_3 | 1.20E+04 | 1.20E+04 | |
| 2_12 | 0.00E+00 | | 0.00E+00 | 6_4 | 0.00E+00 | 0.00E+00 | |
| 3_1 | 4.80E+04 | | 4.80E+04 | 6_5 | 0.00E+00 | 0.00E+00 | 100 |
| 57.1 | | | | 6_6 | 0.00E+00 | 0.00E+00 | |
| 3_2 | 2.40E+04 | 2.40E+04 | | 6_7 | 0.00E+00 | 0.00E+00 | |
| 3_5 | 2.40E+04 | 2.40E+04 | 28.6 | 6_8 | | | |
| | 0.00E+00 | | 0.00E+00 | | | | |

Among 59 samples, 30 samples had countable cells. 10 samples had total cell number above 1×10⁵. Only 5 samples reached viability above 90%.

FSC/SSC Analysis by FACS

Among 59 samples, all the samples showed lots of cell debris by FSC/SSC. None of the samples were found to have the same distribution pattern as granulocytes, lymphocytes, monocytes and macrophages, suggesting that there were no visible granulocytes, lymphocytes, monocytes or macrophages in the tested samples (FIG. 1 to FIG. 9).

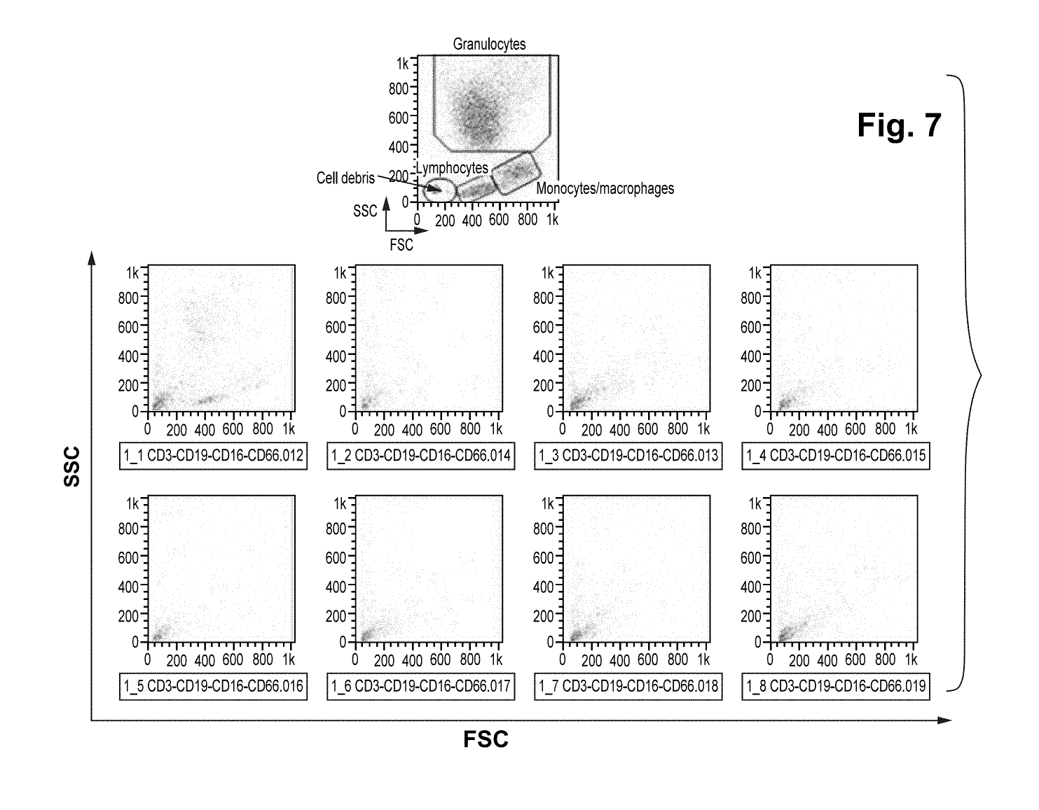

FIG. 7. FSC/SSC on FACS

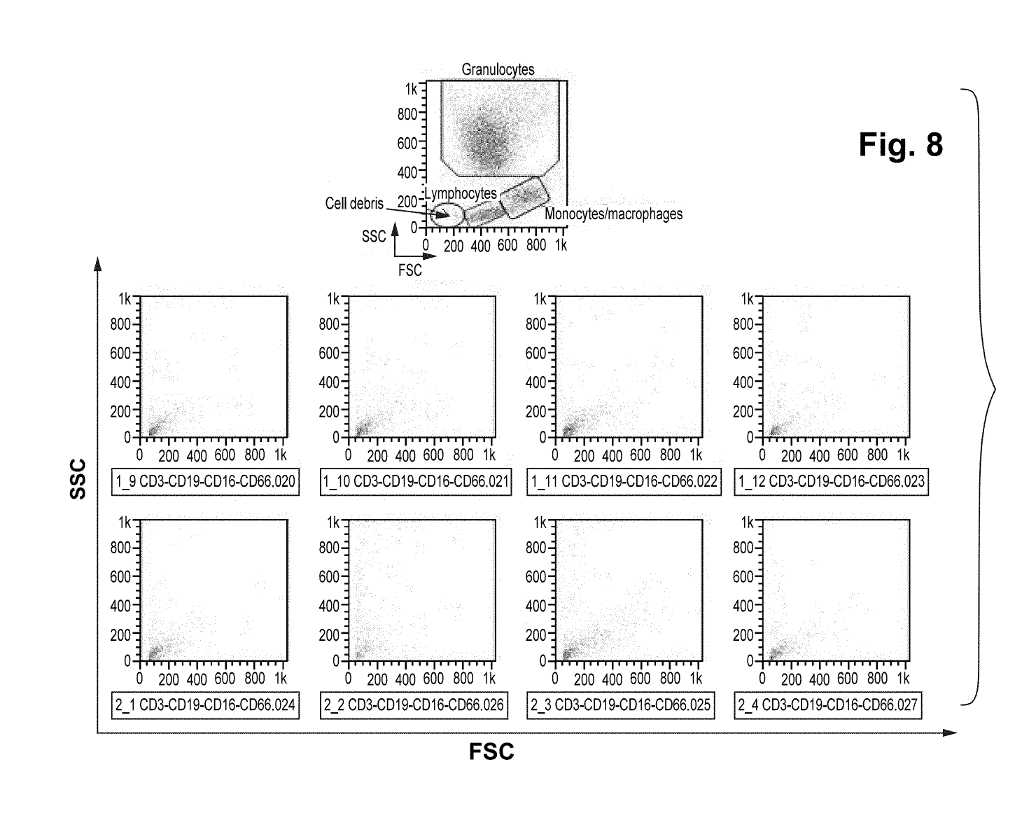

FIG. 8. FSC/SSC on FACS

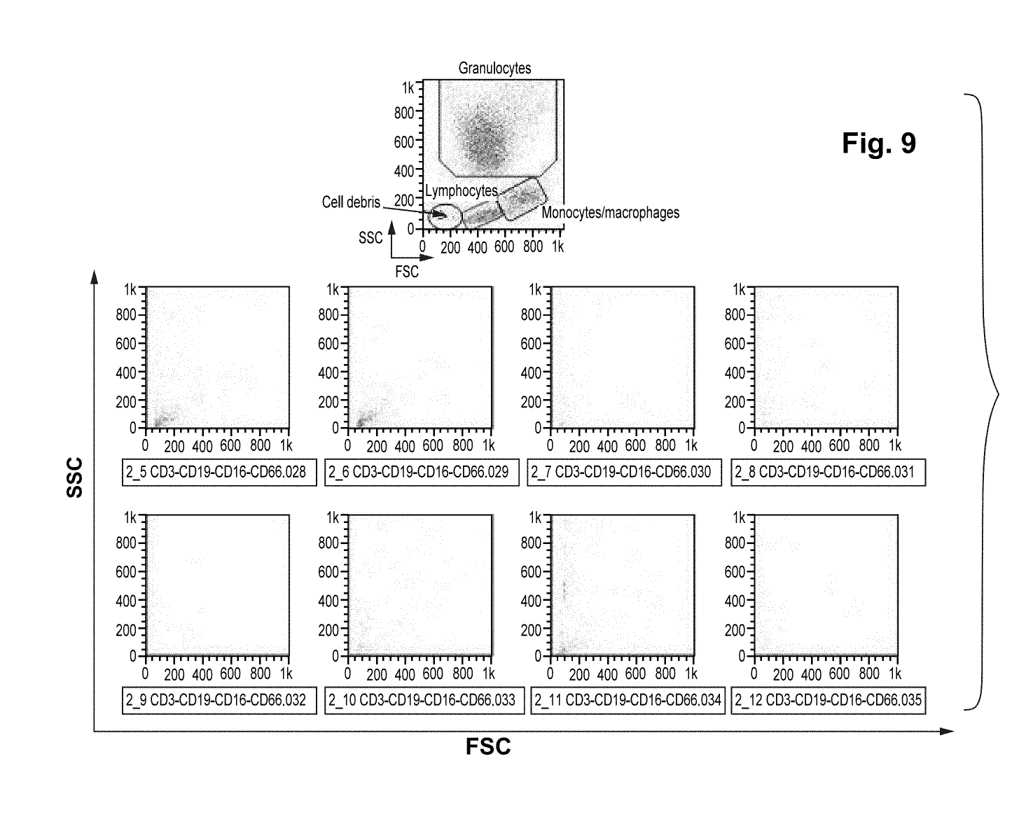

FIG. 9. FSC/SSC on FACS

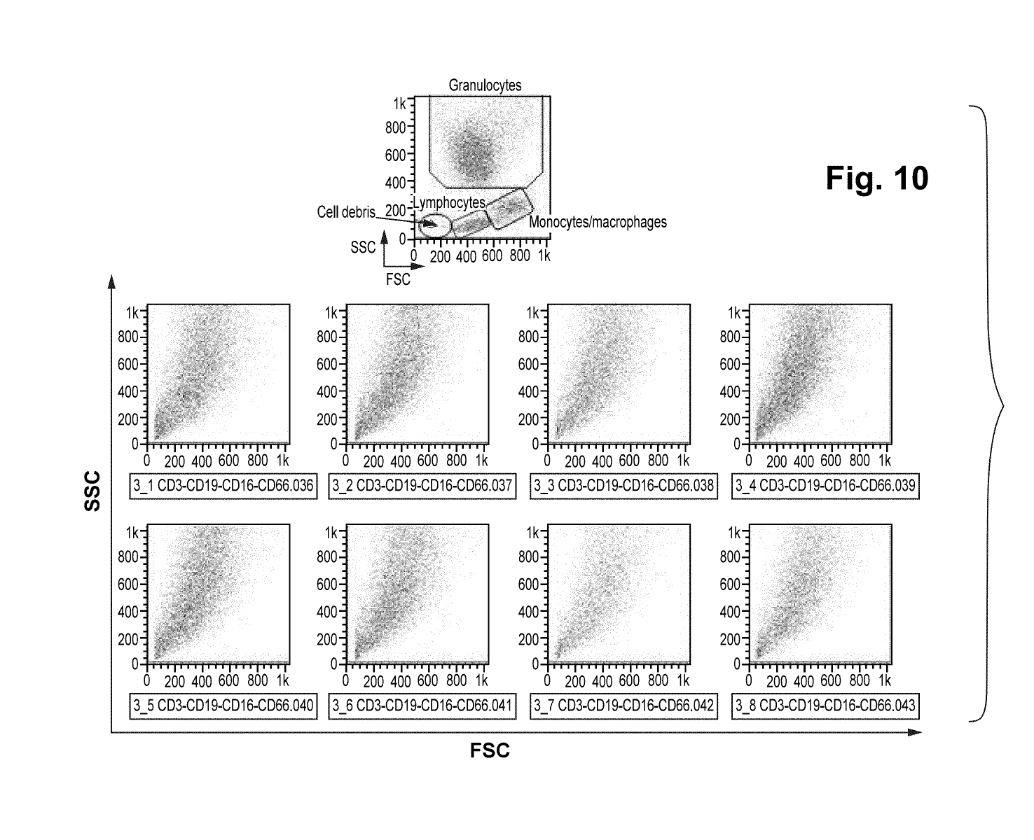

FIG. 10. FSC/SSC on FACS

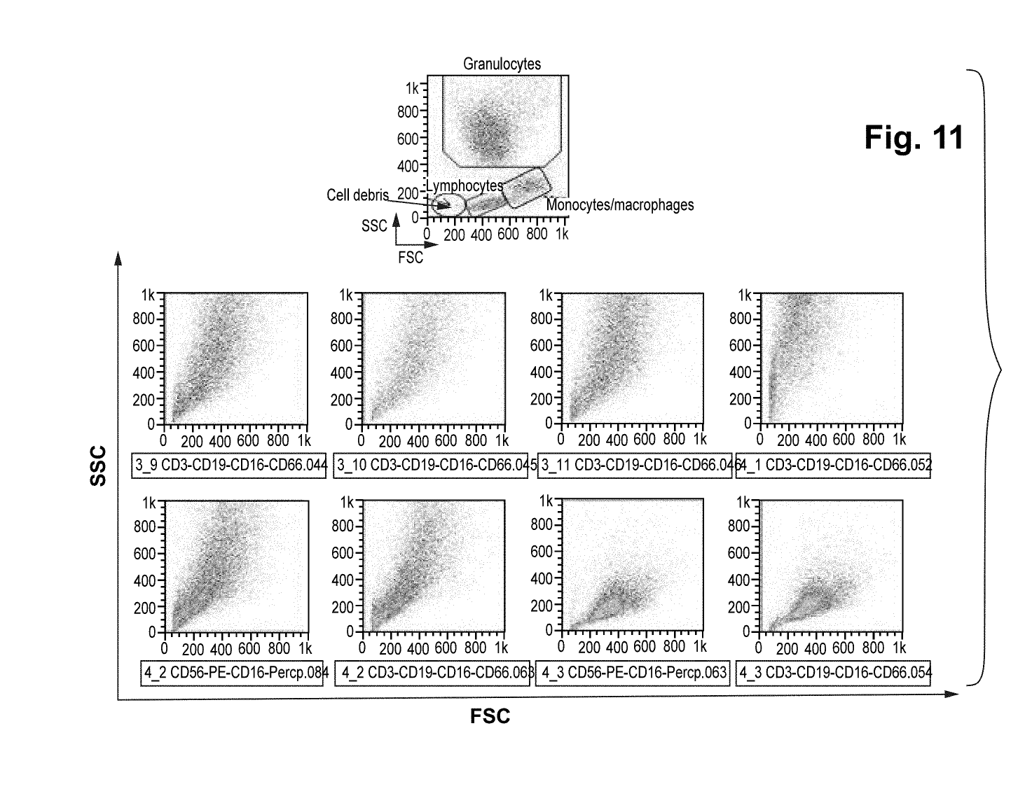

FIG. 11. FSC/SSC on FACS

FIG. 1Z FSC/SSC on FACS

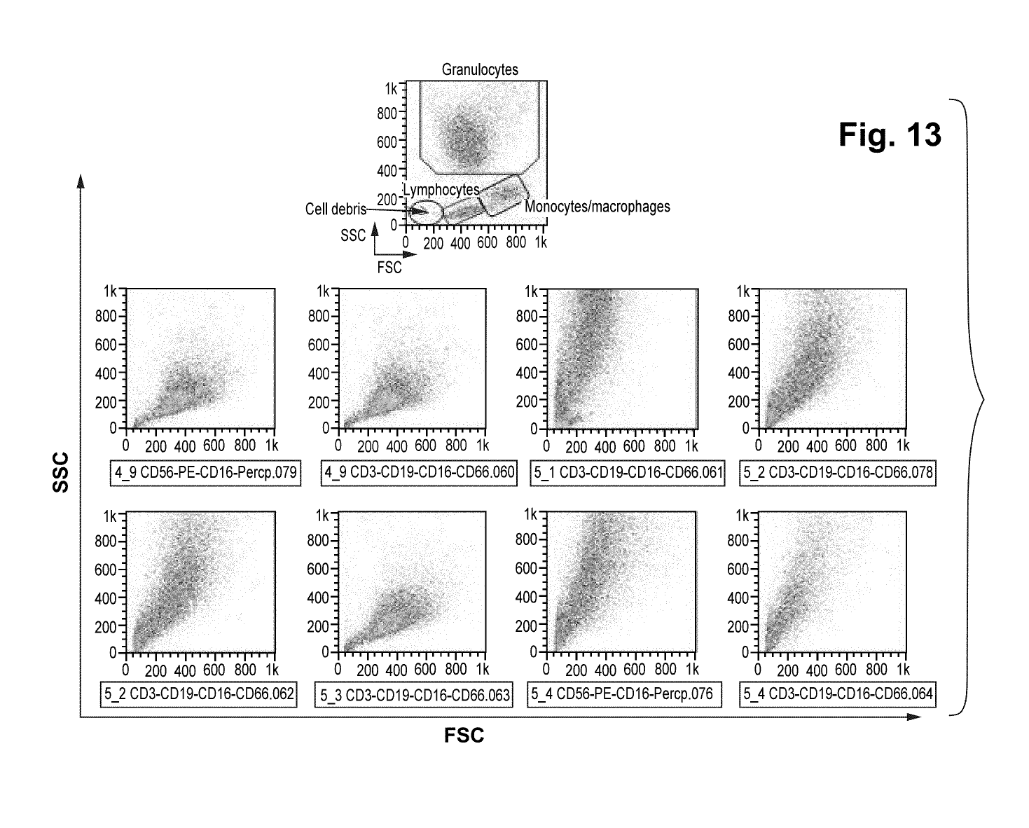

FIG. 13. FSC/SSC on FACS

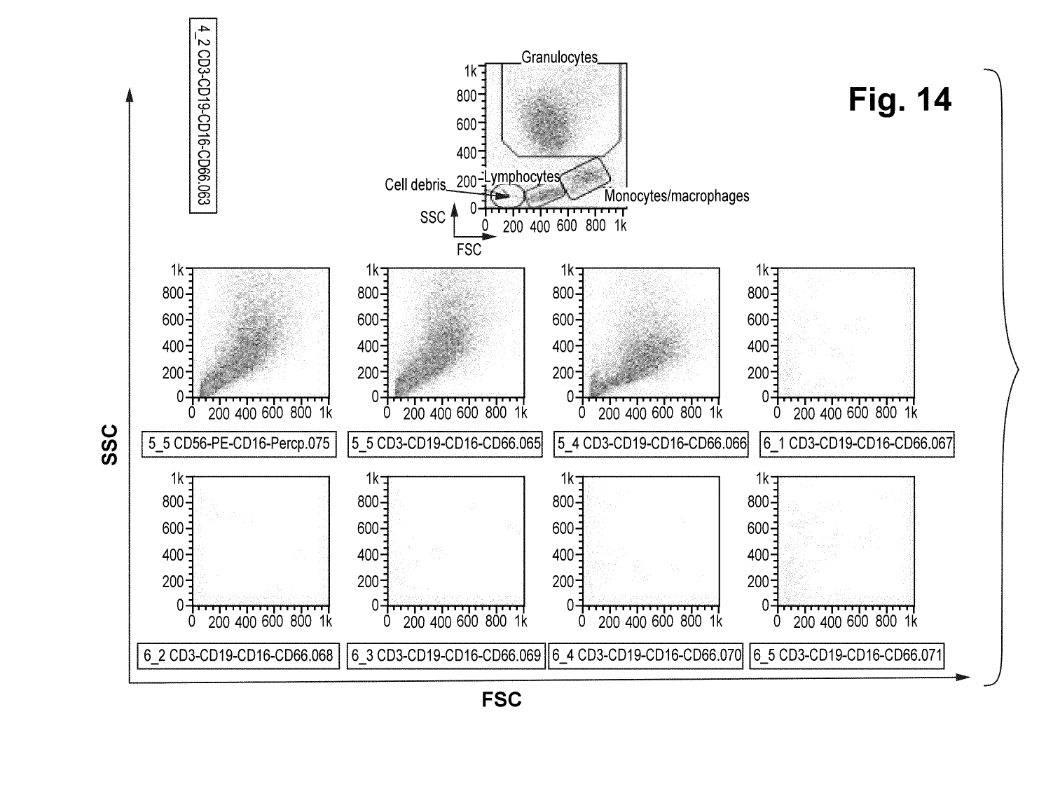

FIG. 14. FSC/SSC on FACS

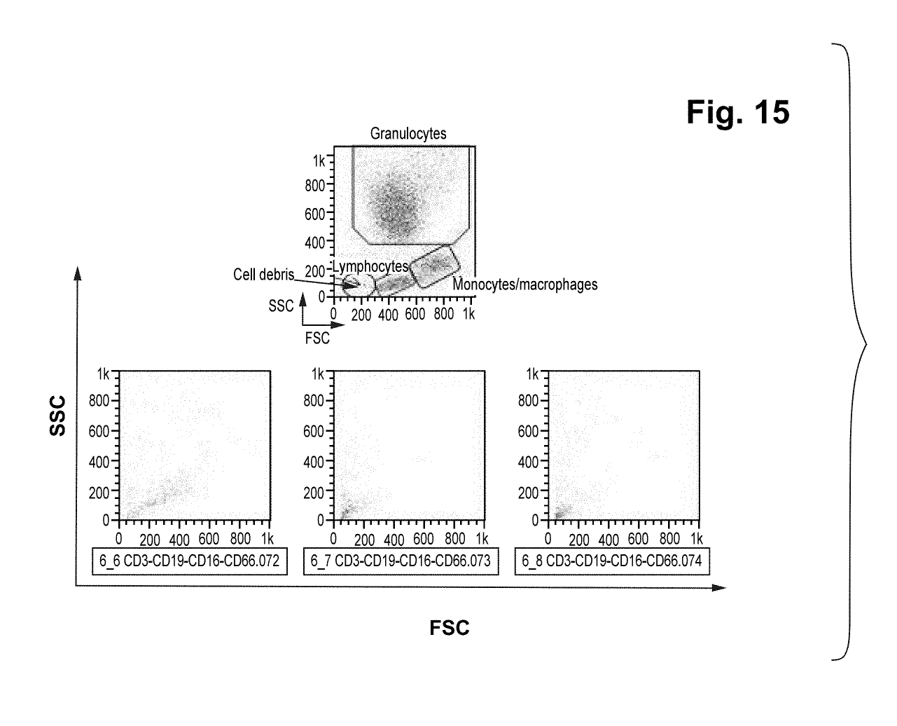

FIG. 15. FSC/SSC on FACS

Comparison with human T/B cells by FACS

Human peripheral blood and test samples were stained side by side with the same antibodies. B and T cell populations were identified by FACS (FIG. 10 to FIG. 16). The data did not show a convincing population of T or B cells.

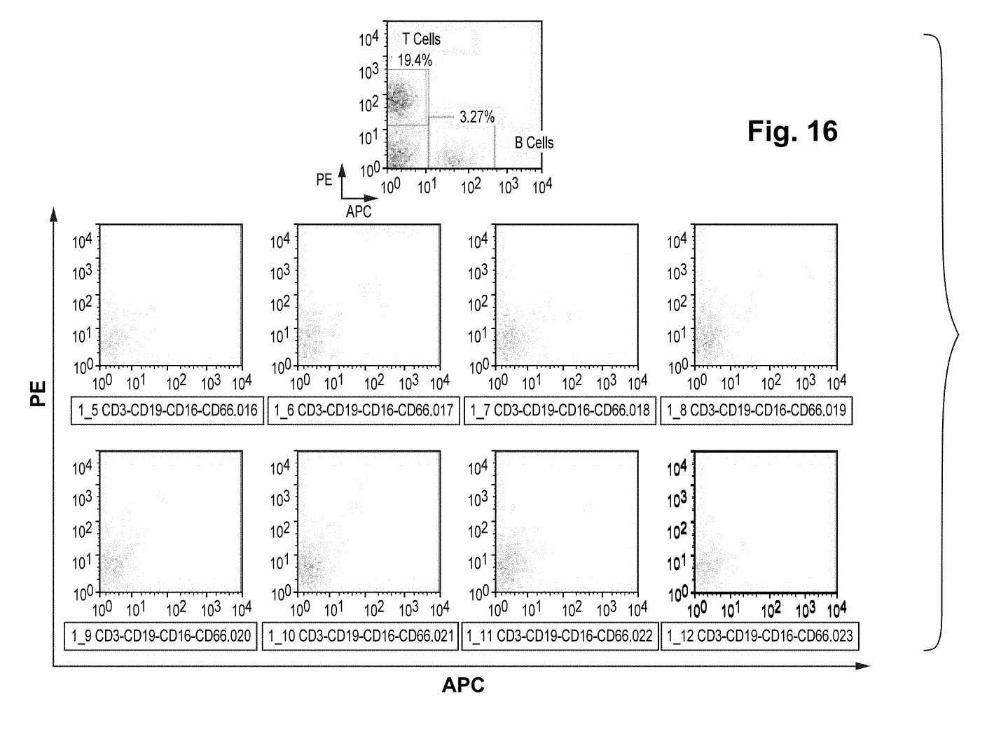

FIG. 16. Comparison with human T/B cells on FACS

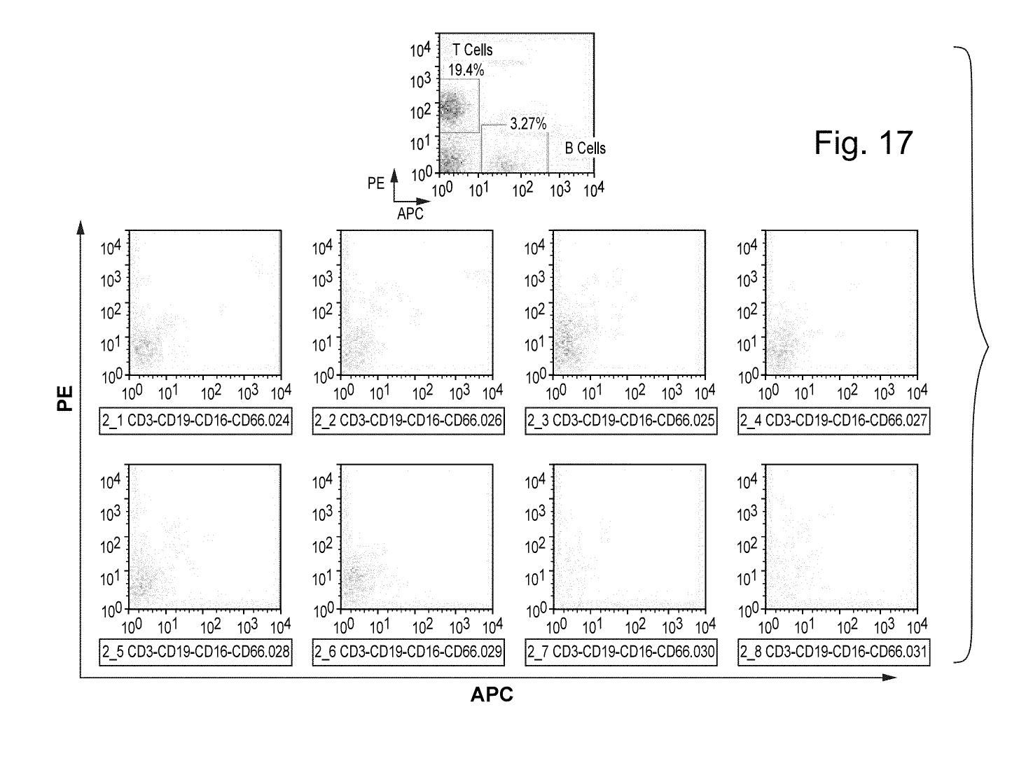

FIG. 17. Comparison with human T/B cells on FACS

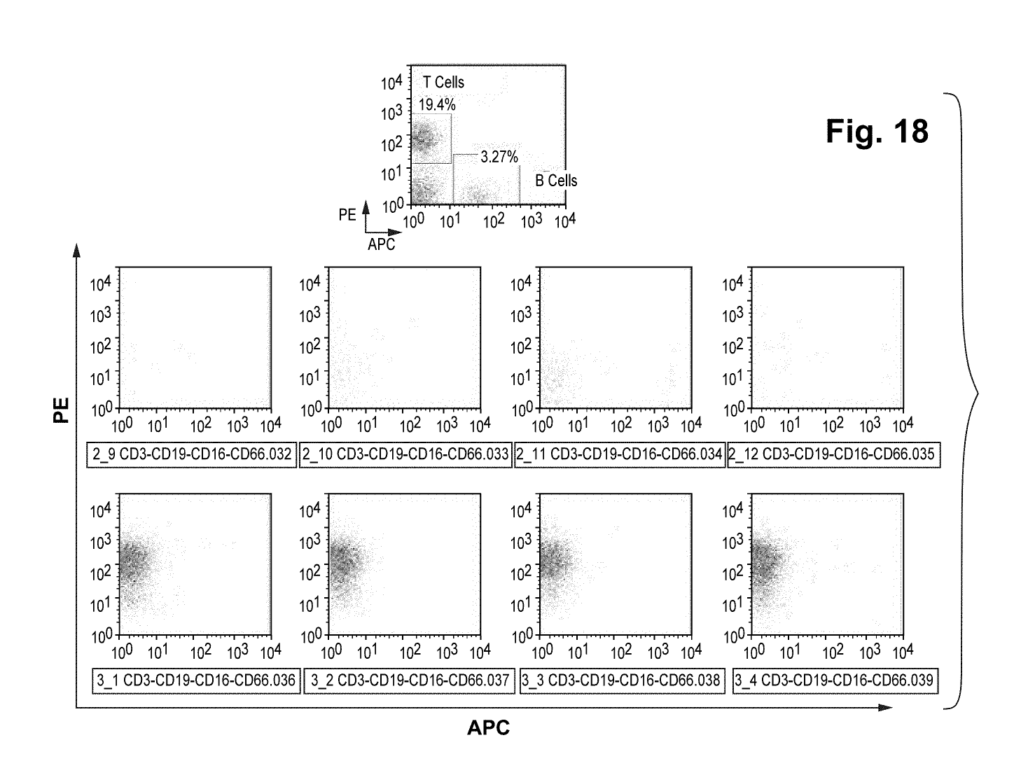

FIG. 18. Comparison with human T/B cells on FACS

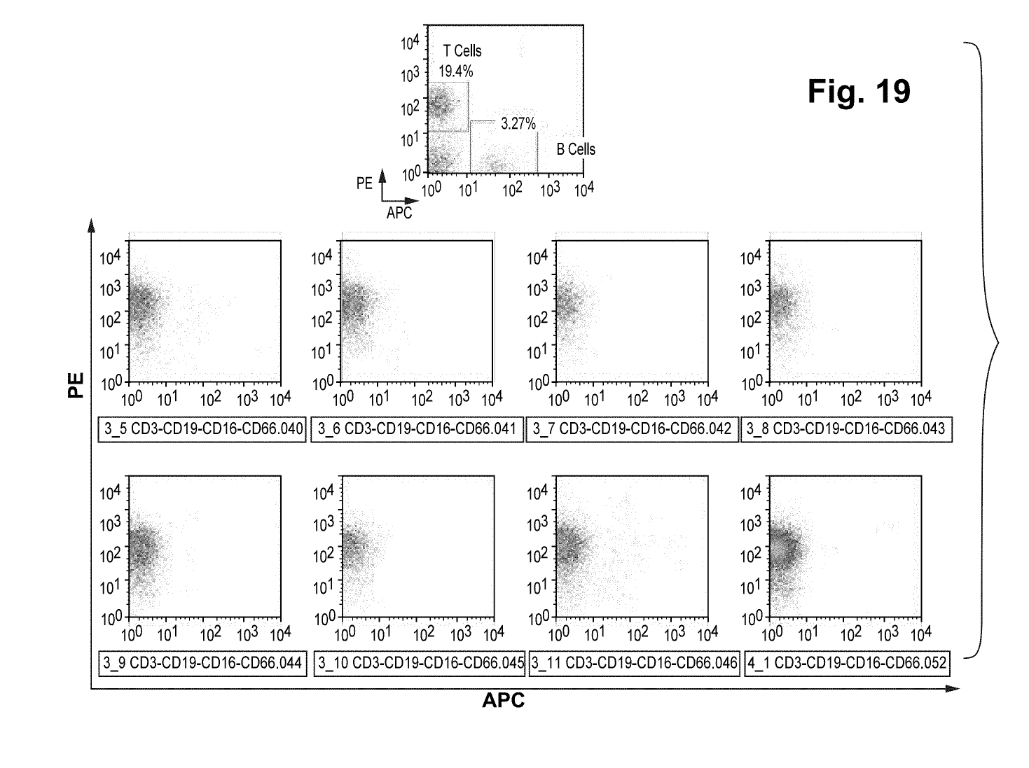

FIG. 19. Comparison with human T/B cells on FACS

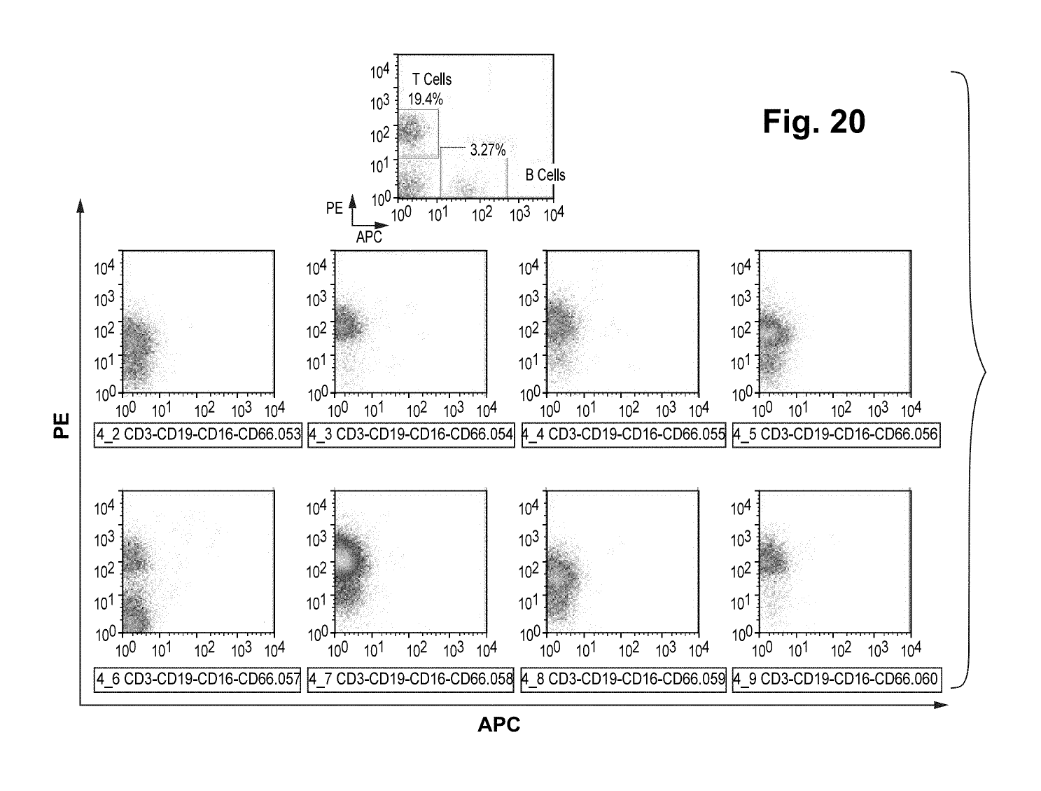

FIG. 20. Comparison with human T/B cells on FACS

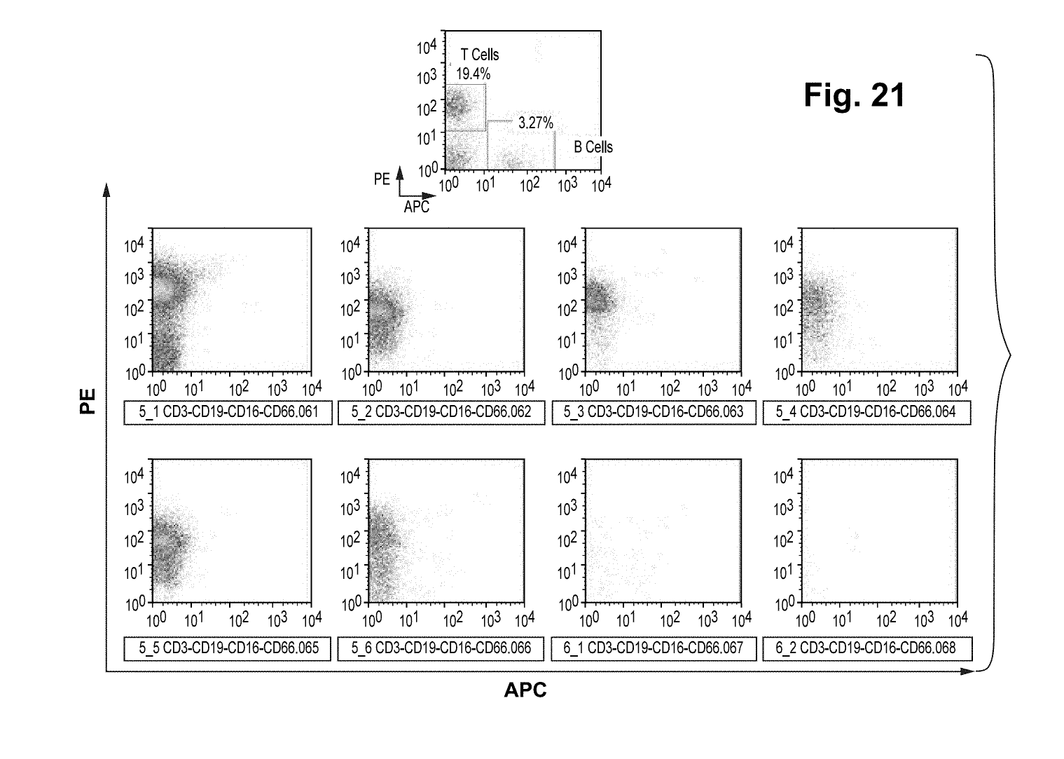

FIG. 21. Comparison with human T/B cells on FACS

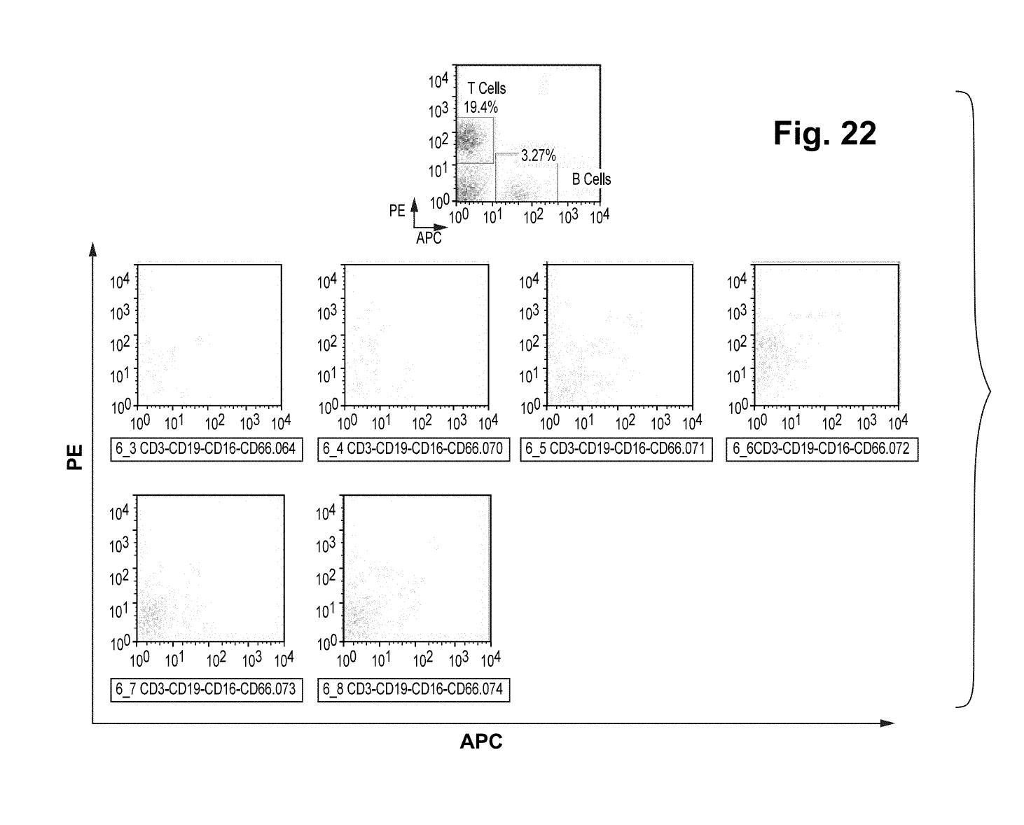

FIG. 22. Comparison with human T/B cells on FACS

Comparison unknown samples with granulocytes by FACS

In addition to staining of T and B lymphocytes, human peripheral blood and test samples were stained simultaneously with the same antibodies and granulocytes were further identified by FACS. No granulocytes were found in all the test samples (FIG. 17 to FIG. 24).

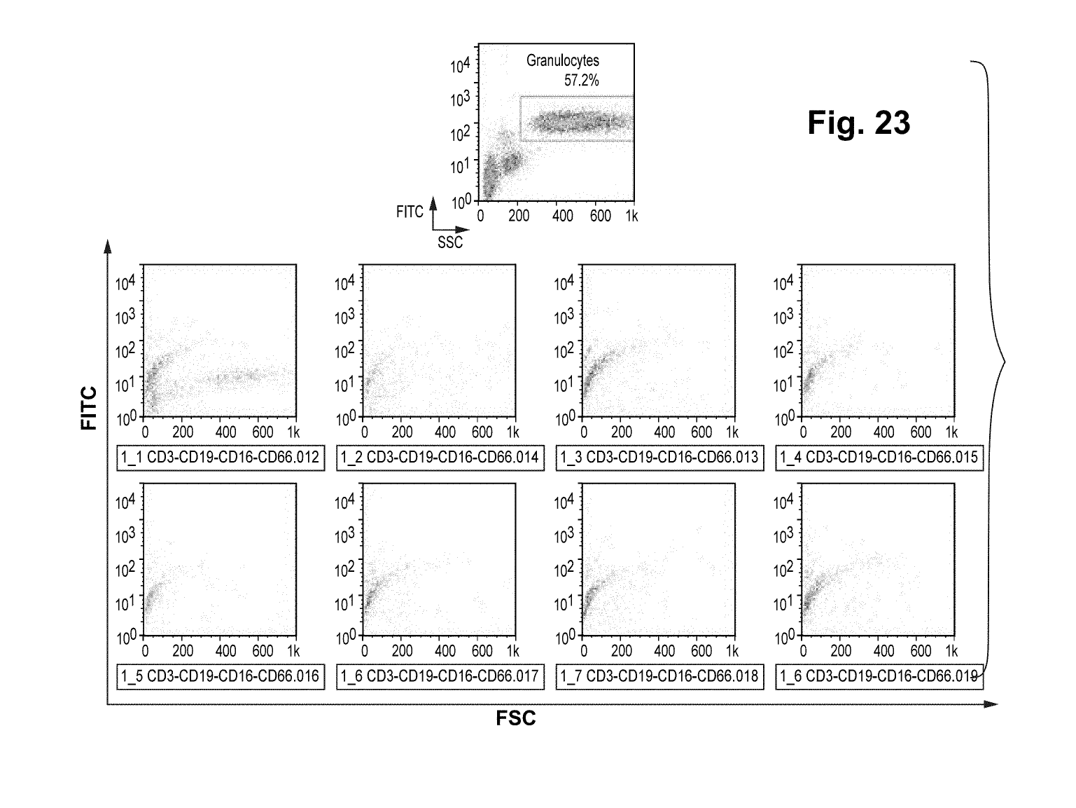

FIG. 23. Comparison with human granulocytes on FACS

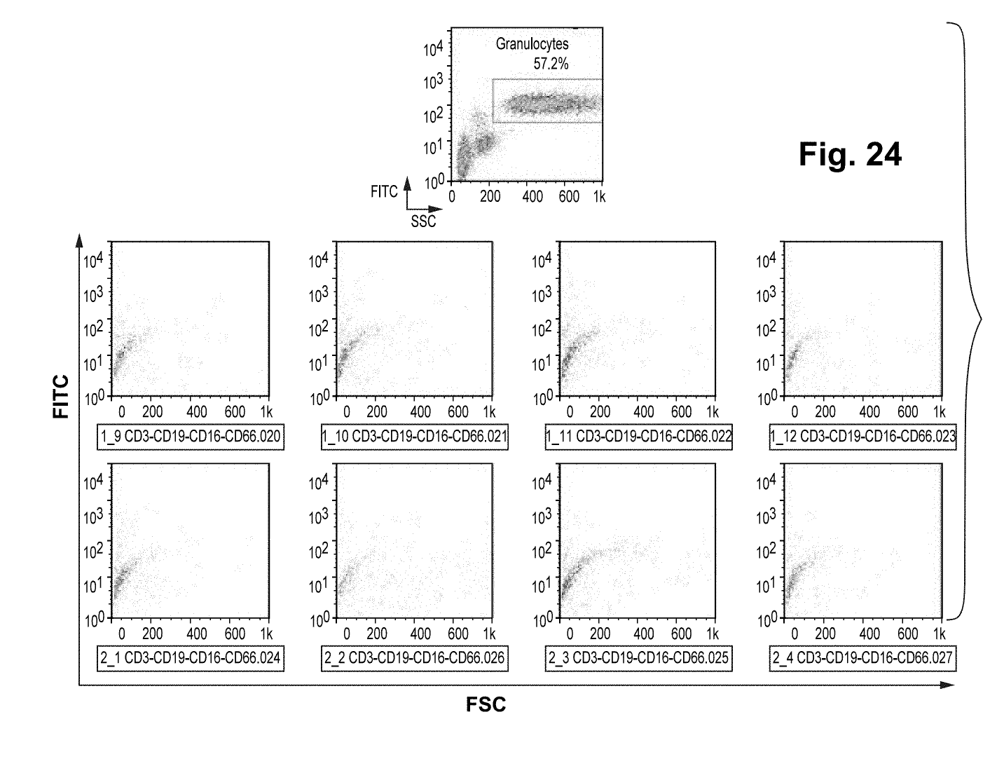

FIG. 24. Comparison with human granulocytes on FACS

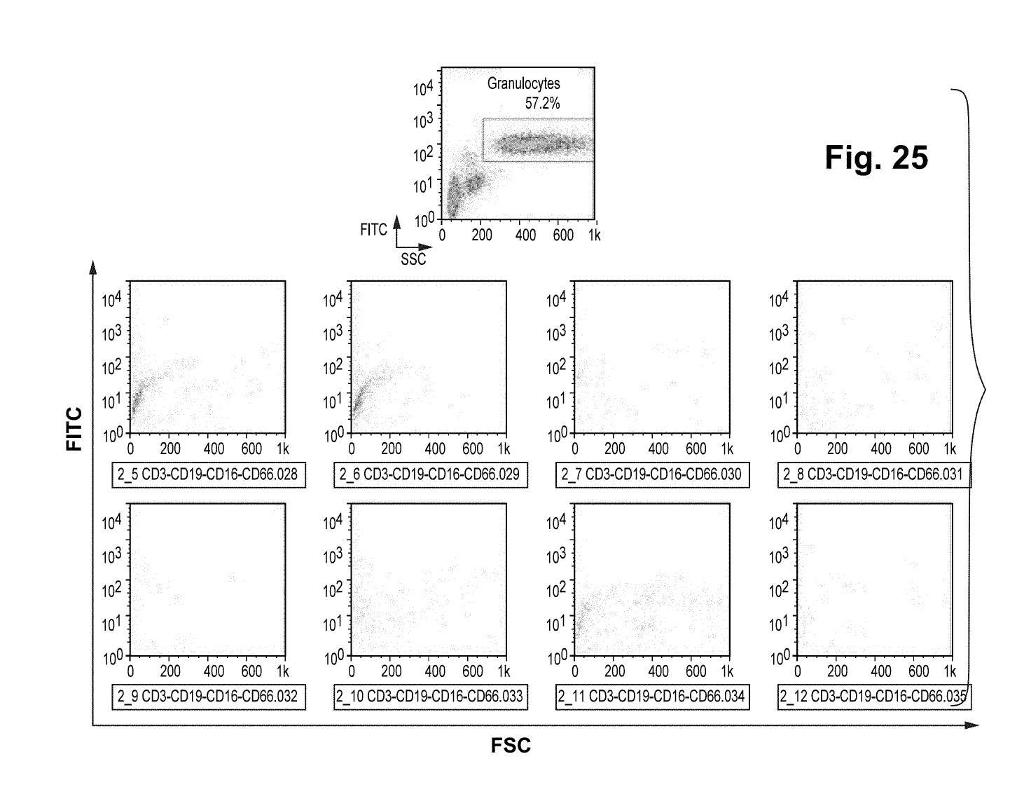

FIG. 25. Comparison with human granulocytes on FACS

FIG. 26. Comparison with human granulocytes on FACS

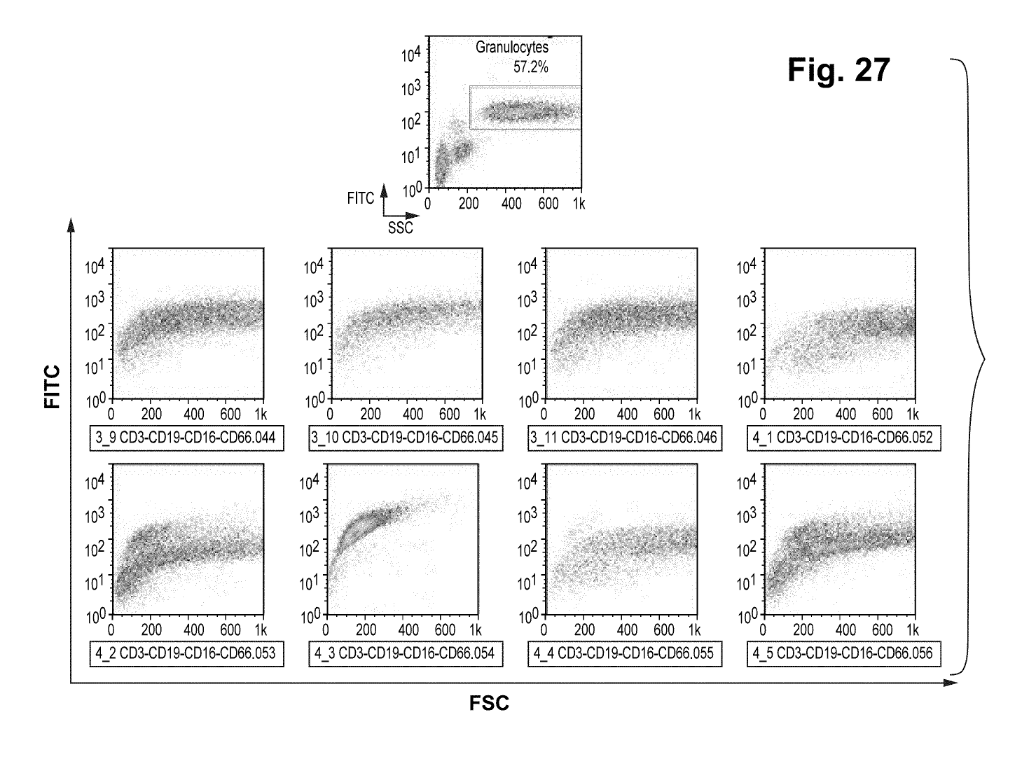

FIG. 27. Comparison with human granulocytes on FACS

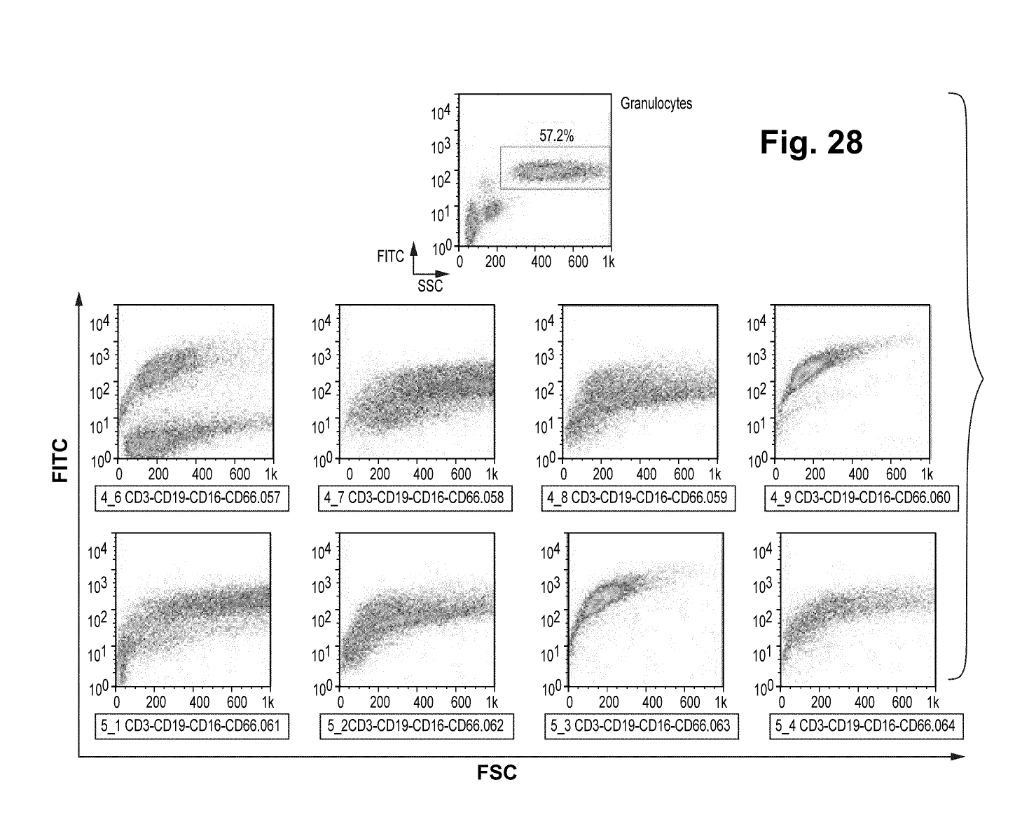

FIG. 28. Comparison with human granulocytes on FACS

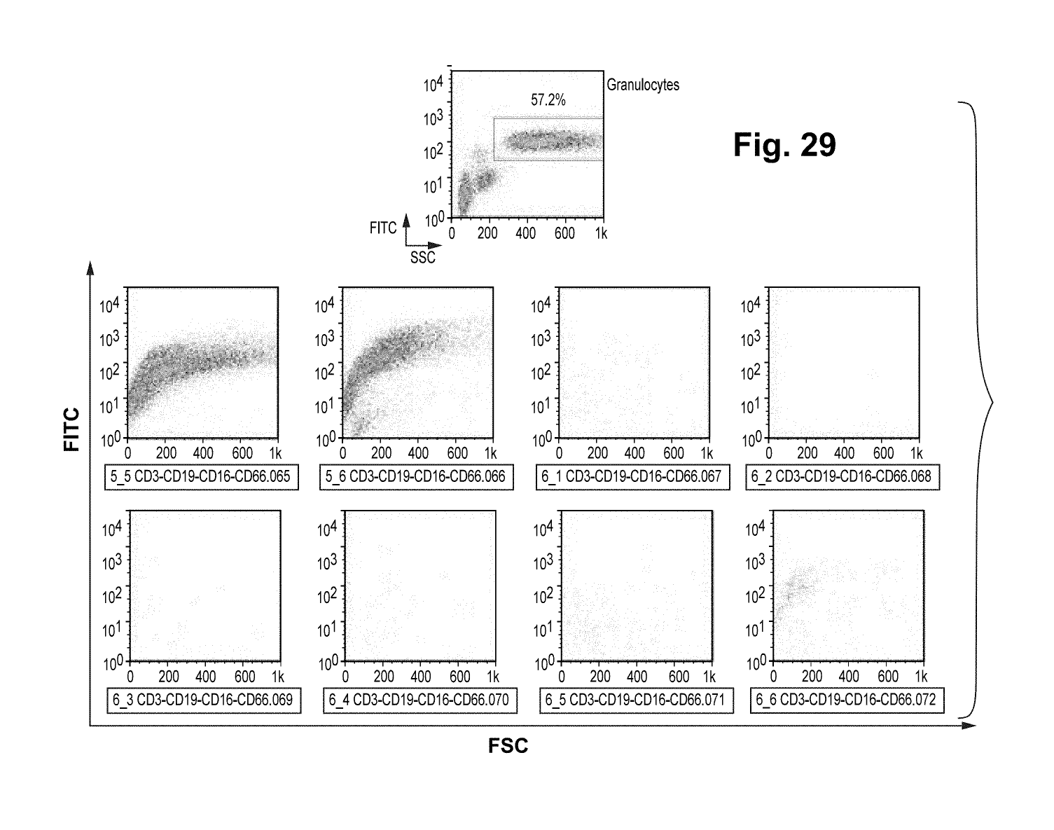

FIG. 29. Comparison with human granulocytes on FACS

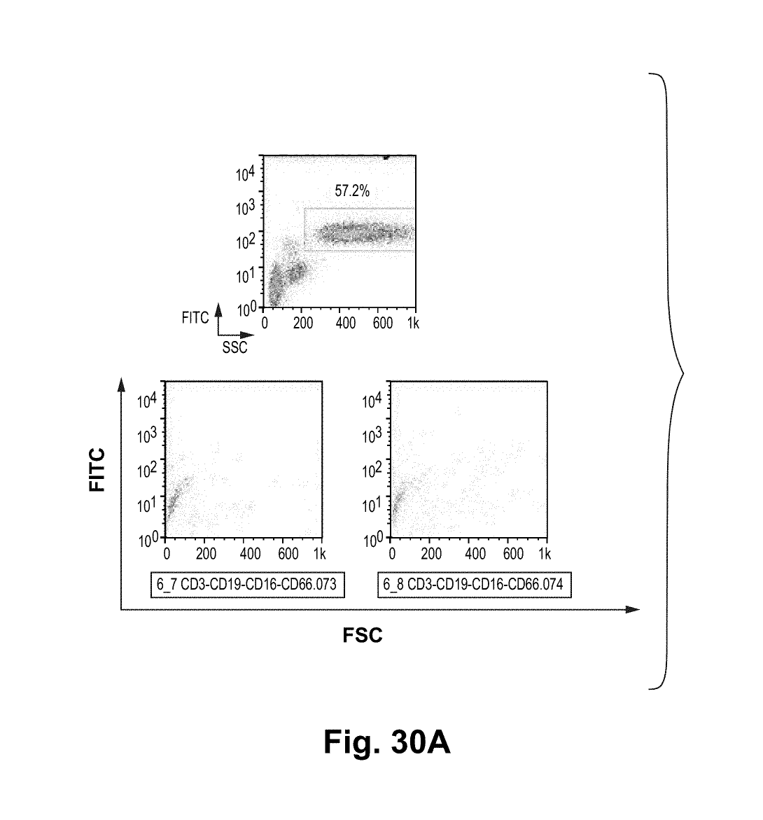

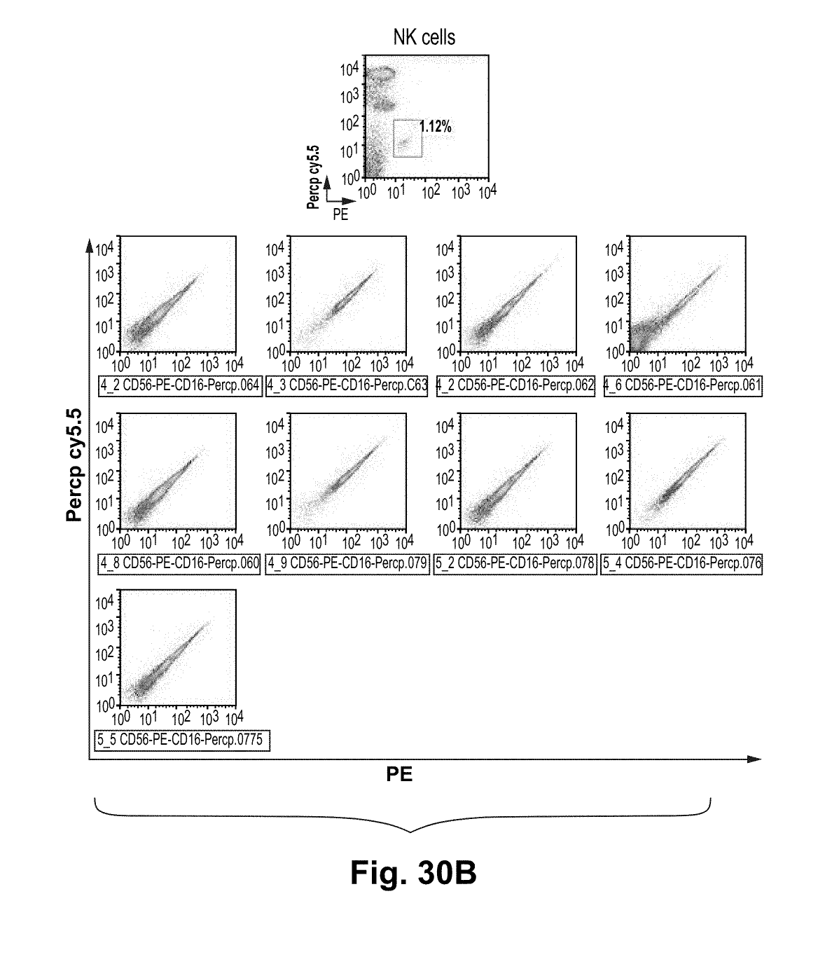

FIG. 30A. Comparison with human granulocytes on FACS

Comparison unknown samples with NK cells by FACS

None of the samples were found to contain NK cells (FIG. 25).

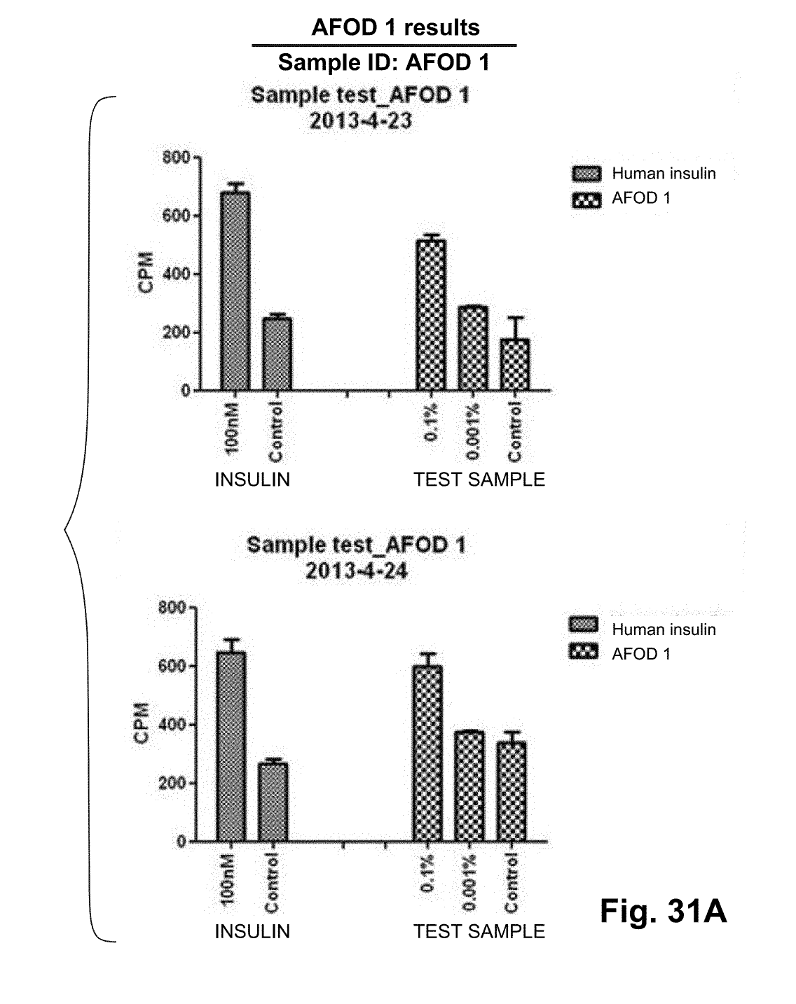

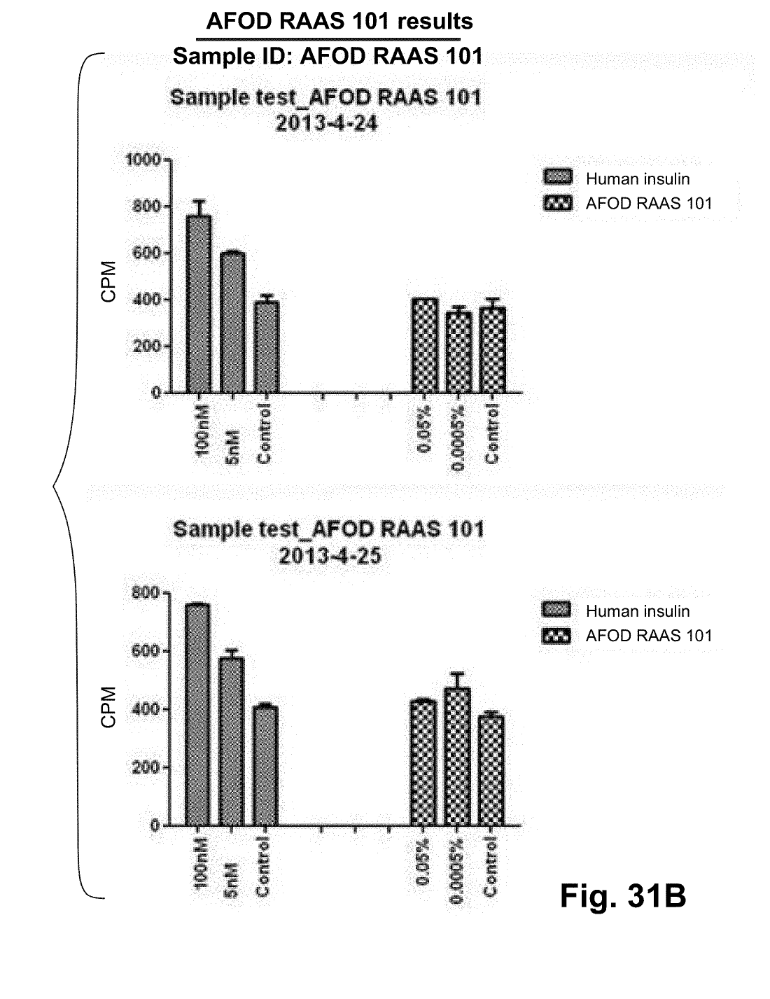

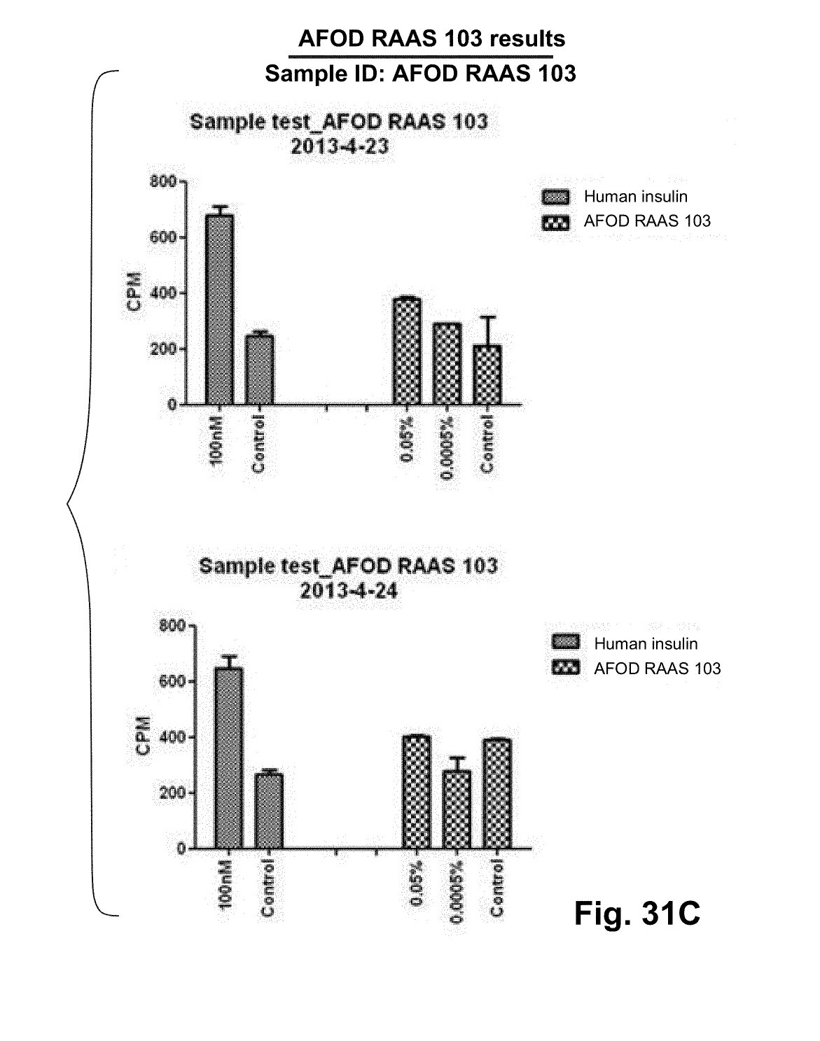

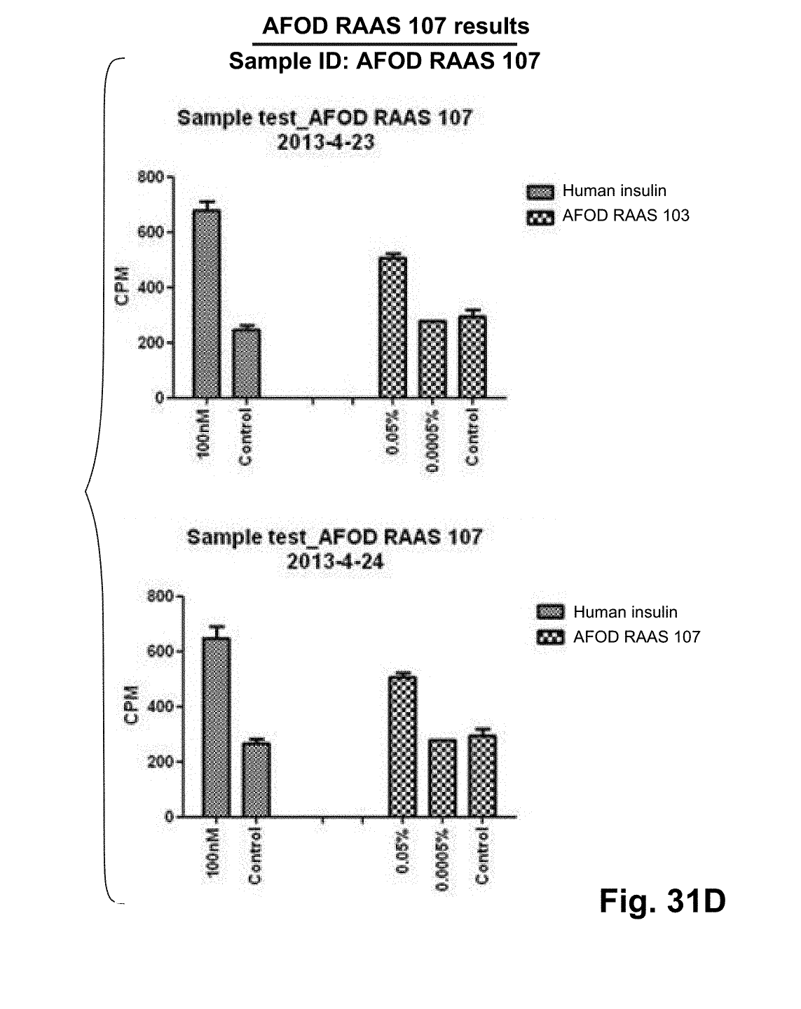

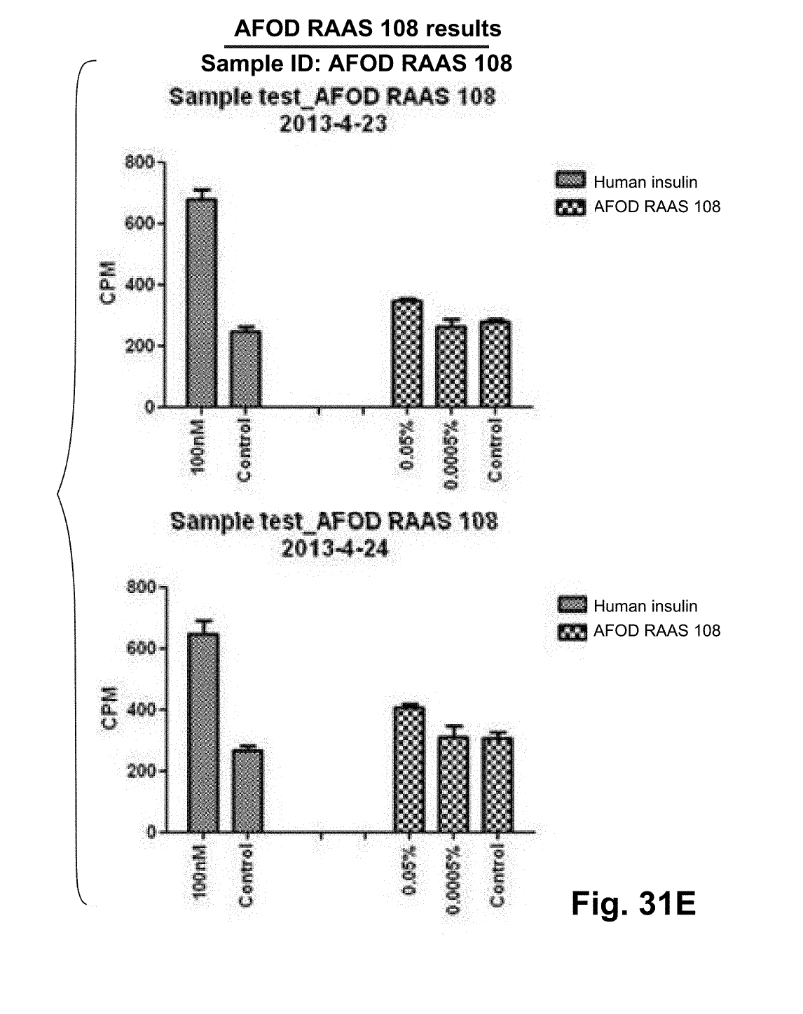

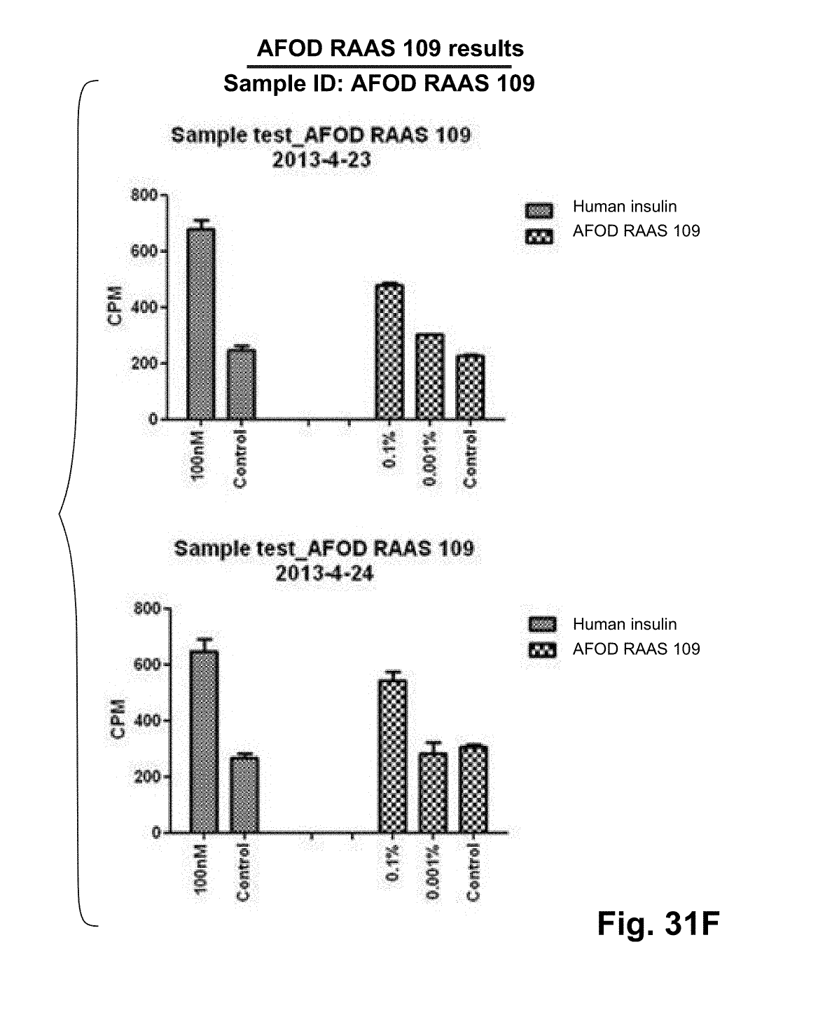

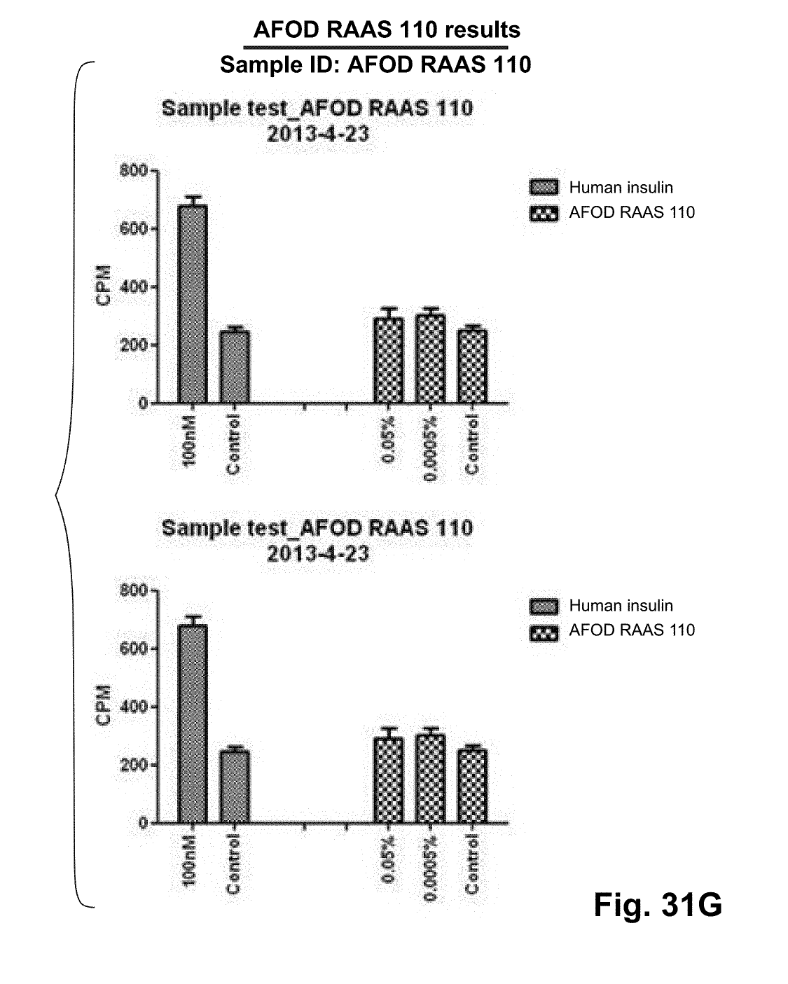

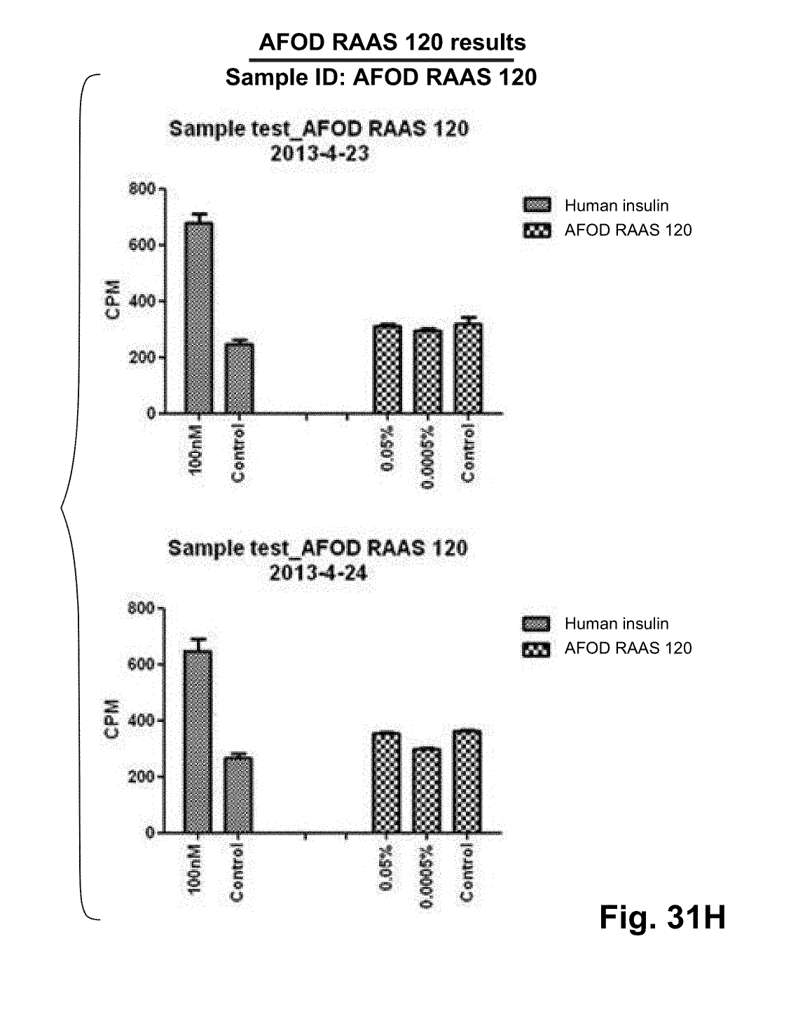

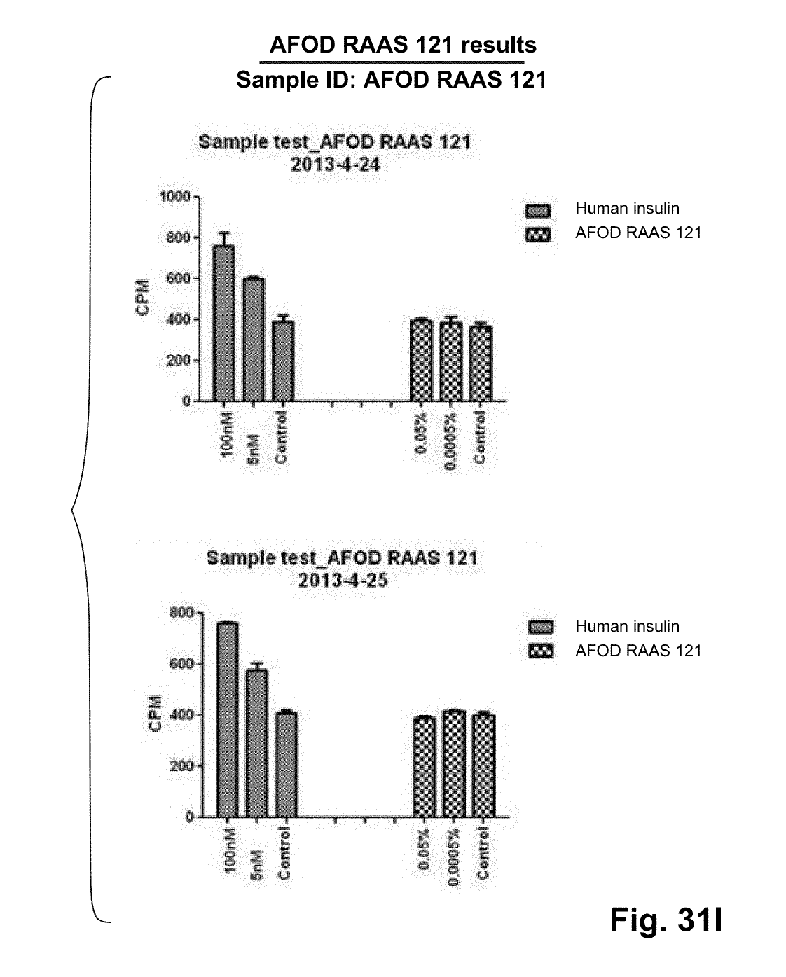

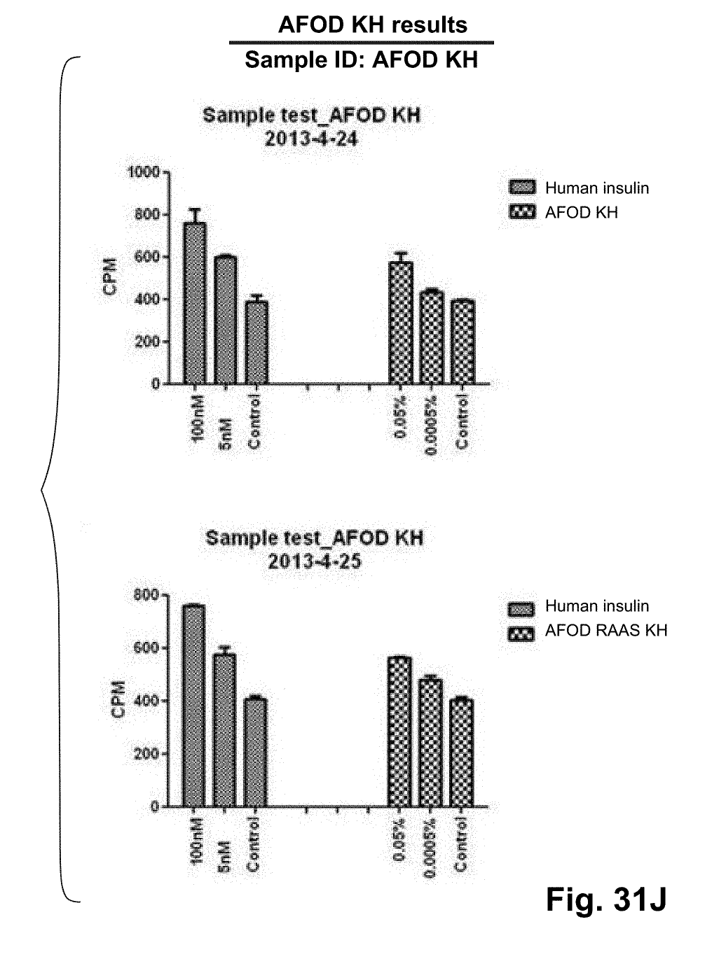

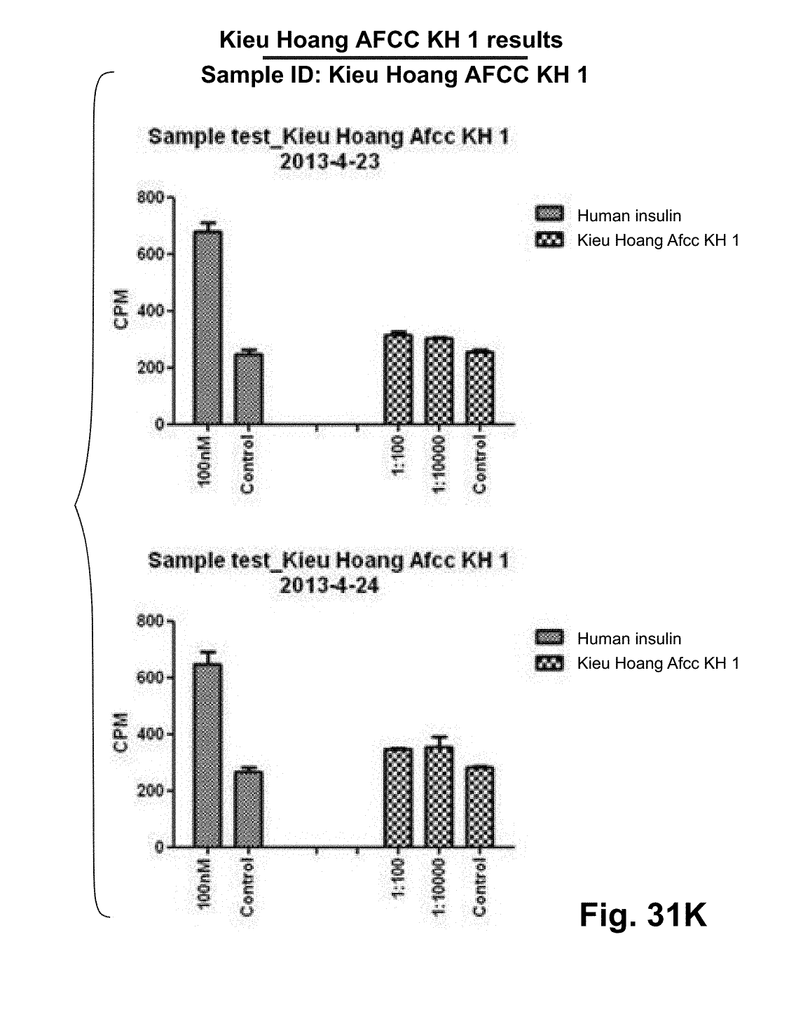

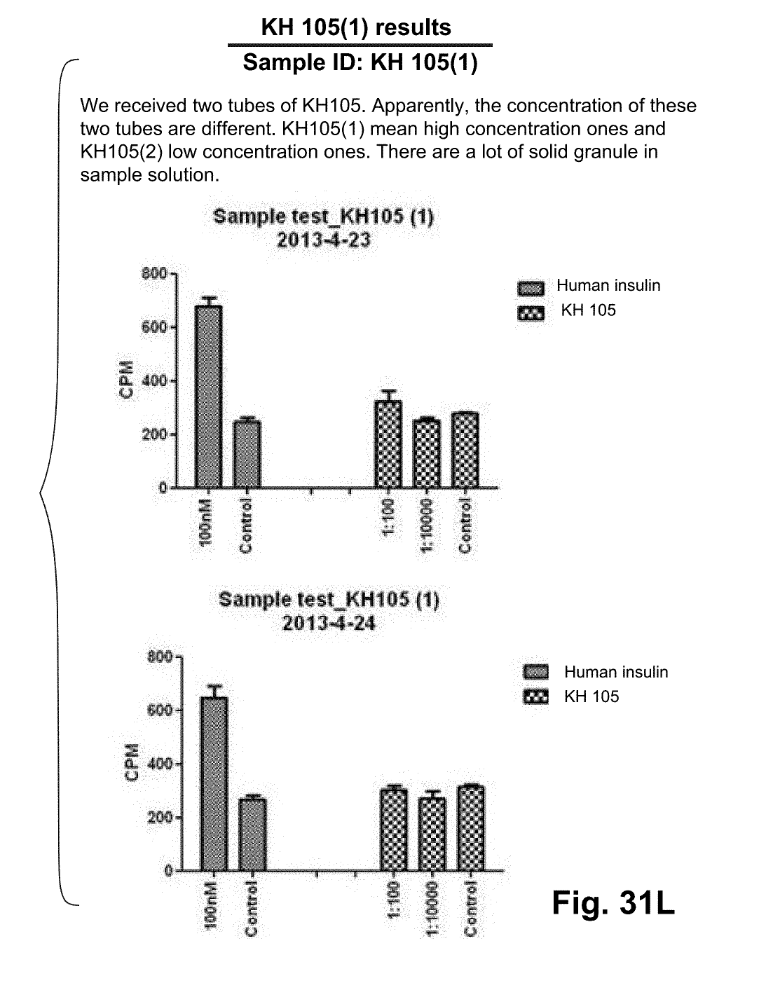

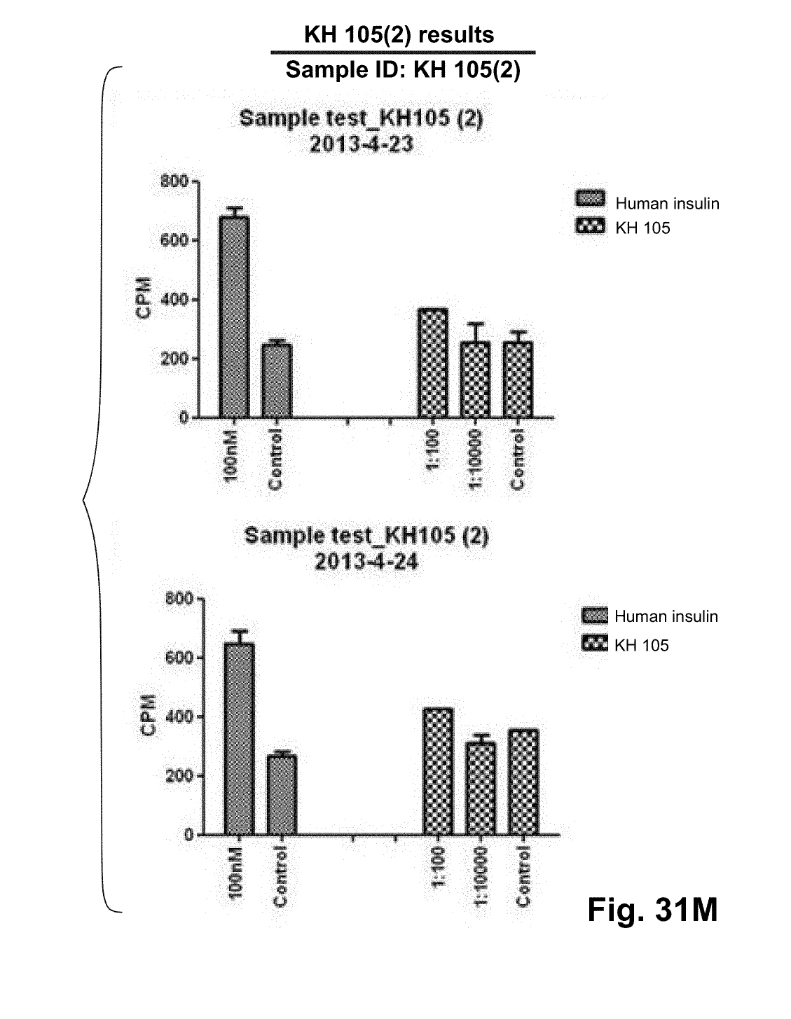

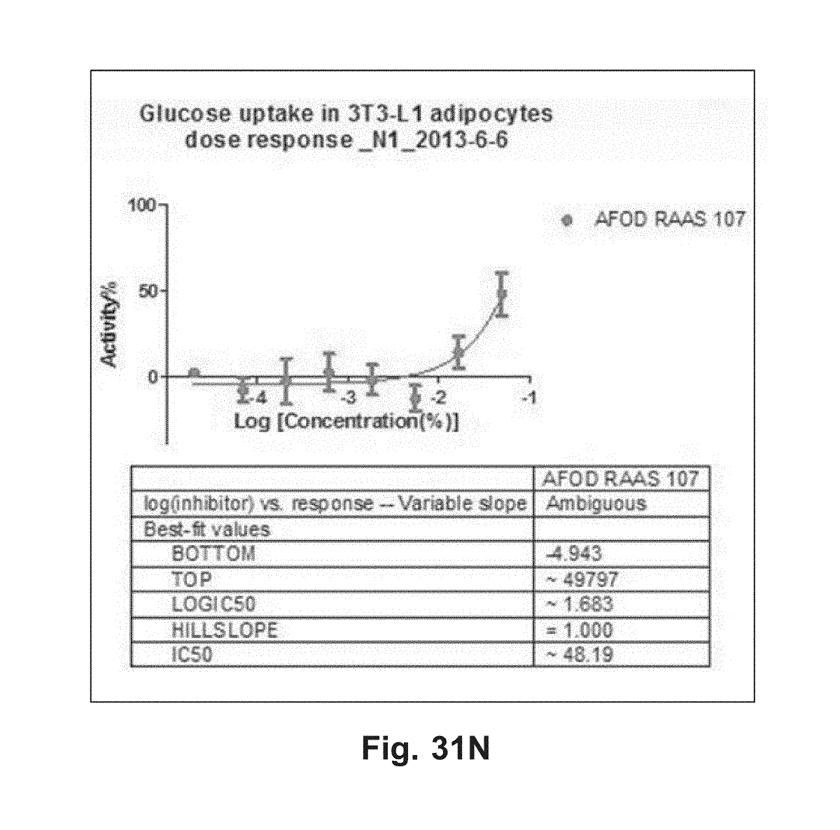

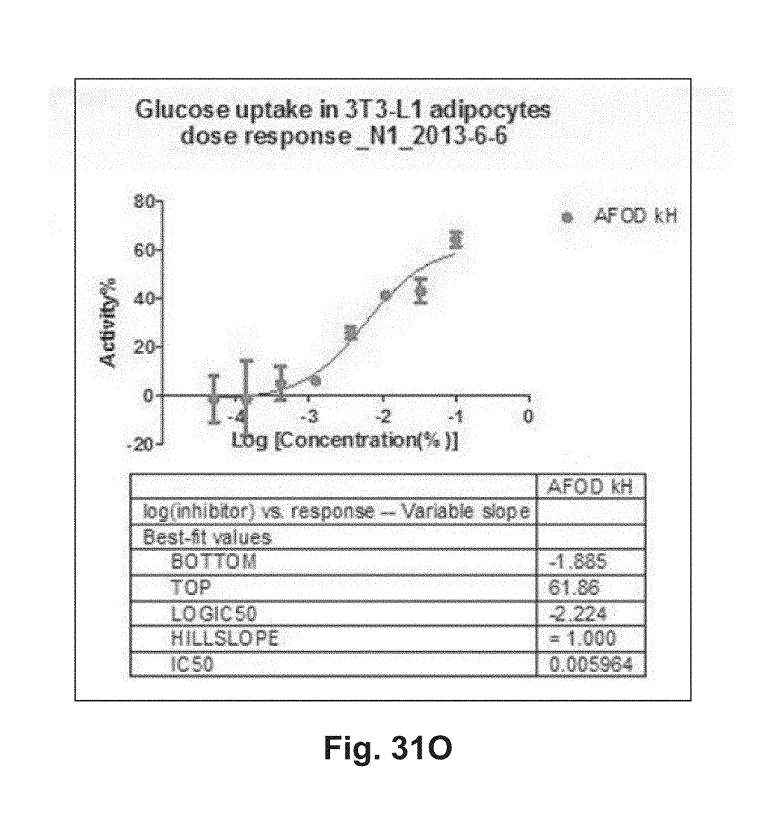

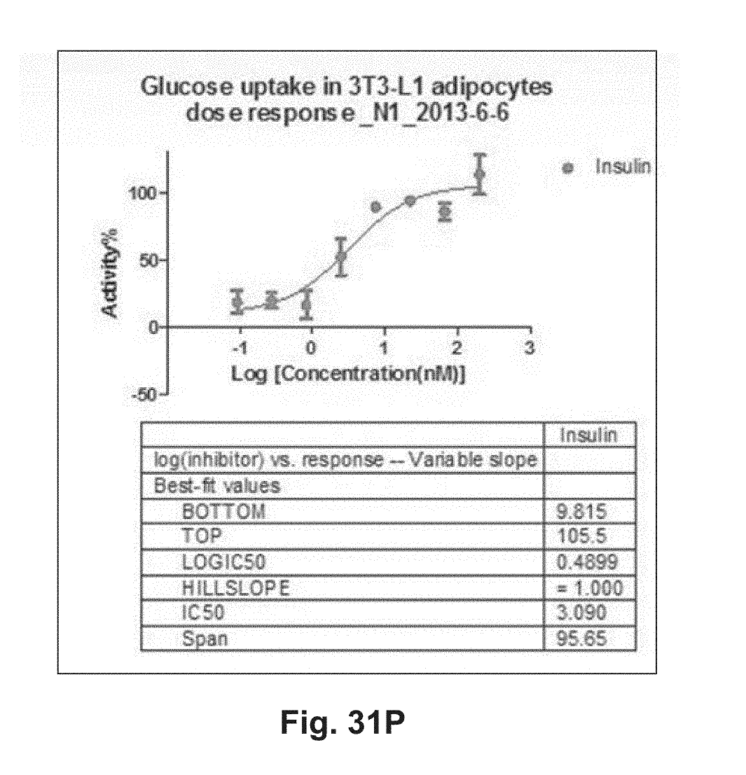

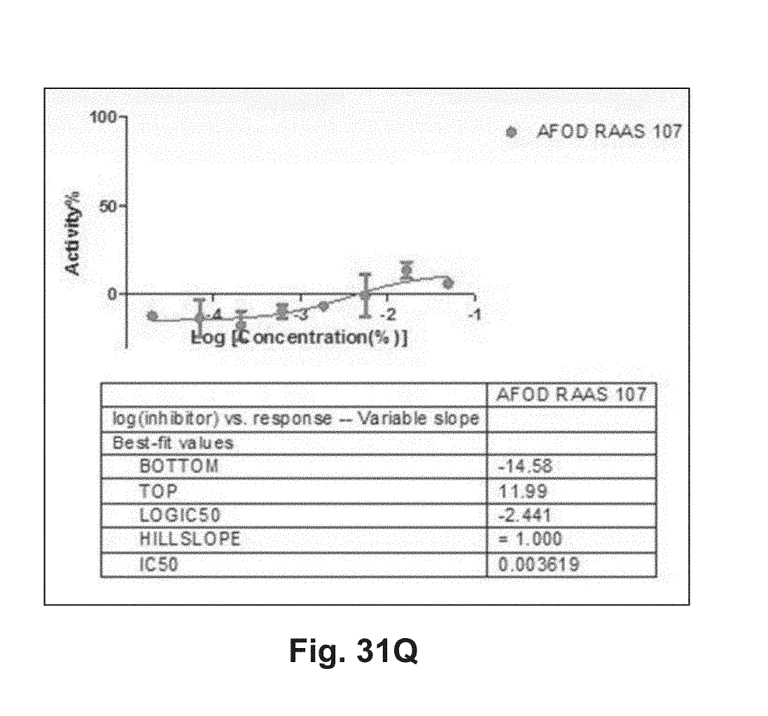

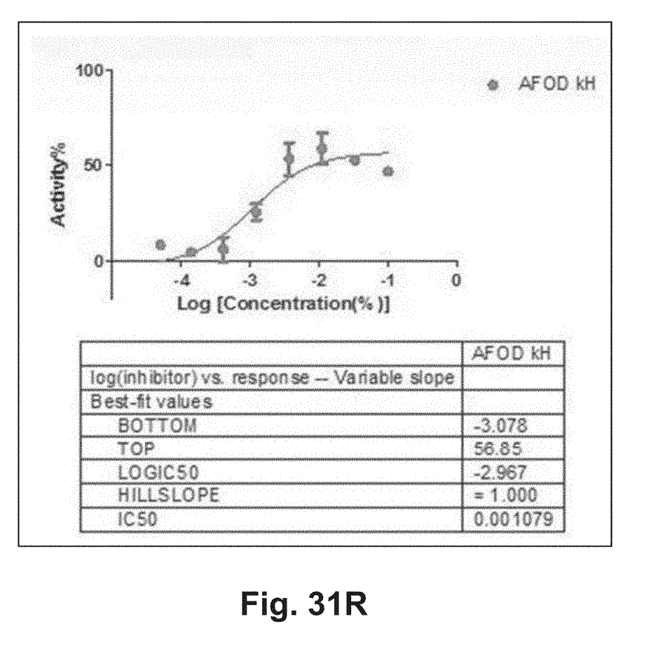

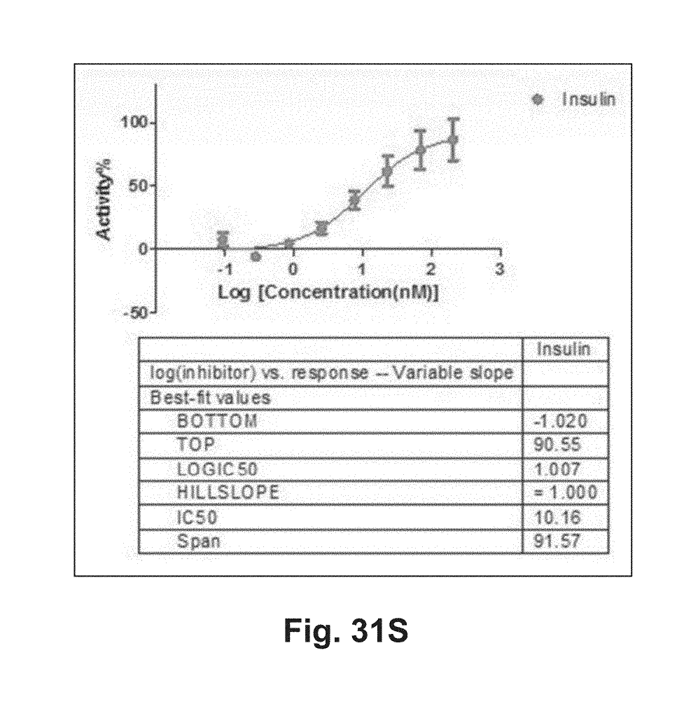

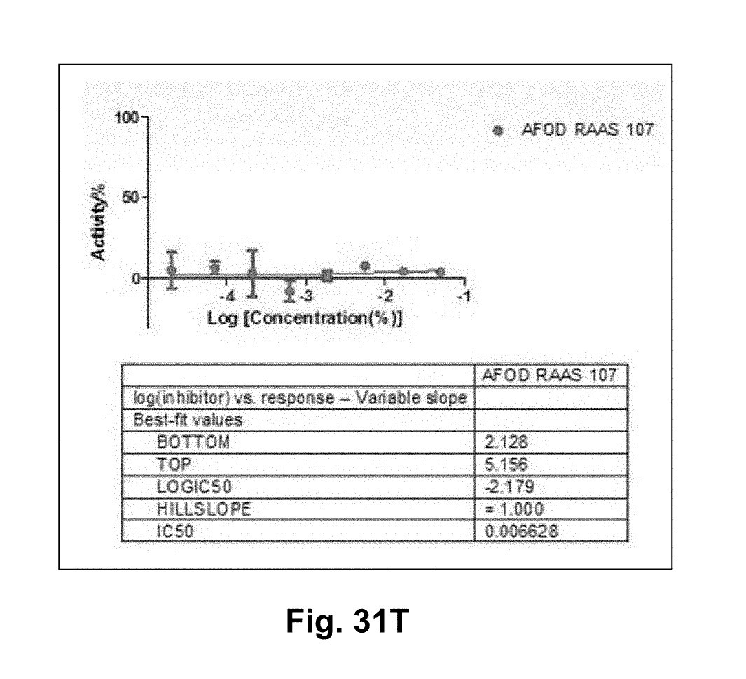

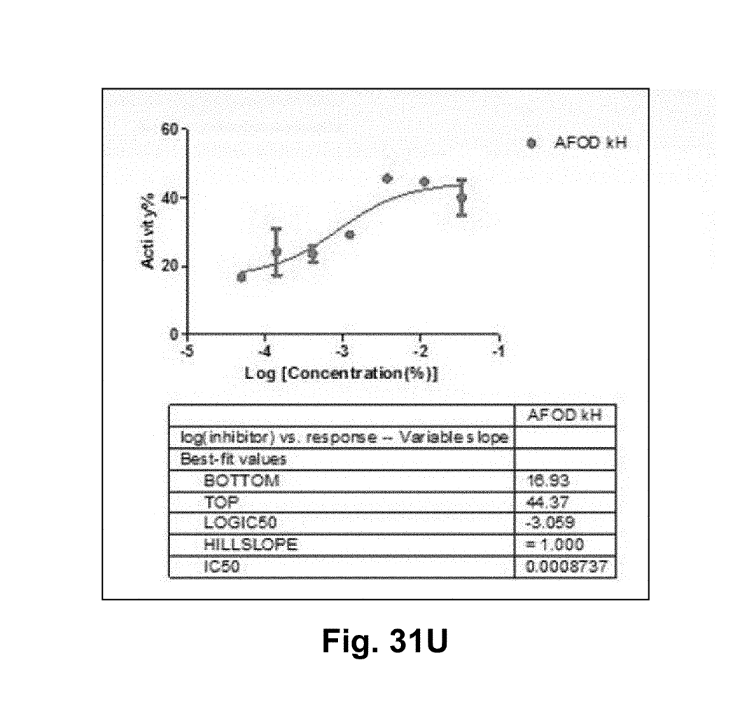

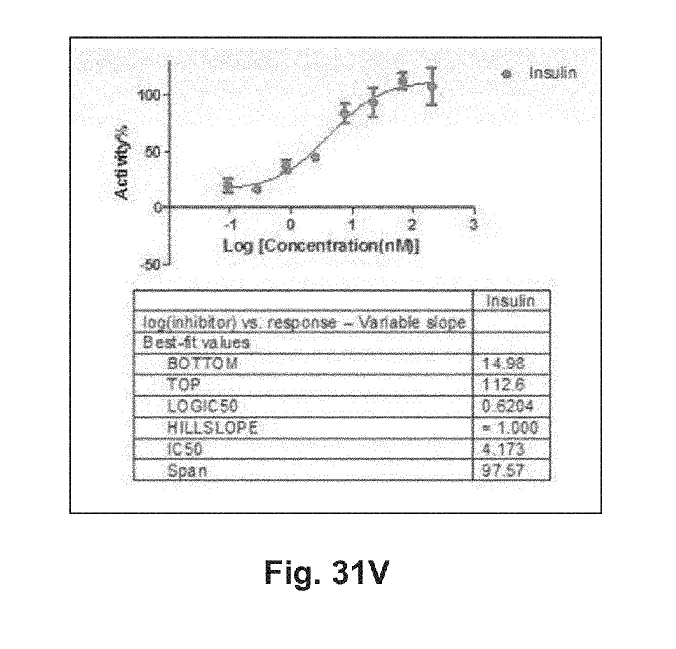

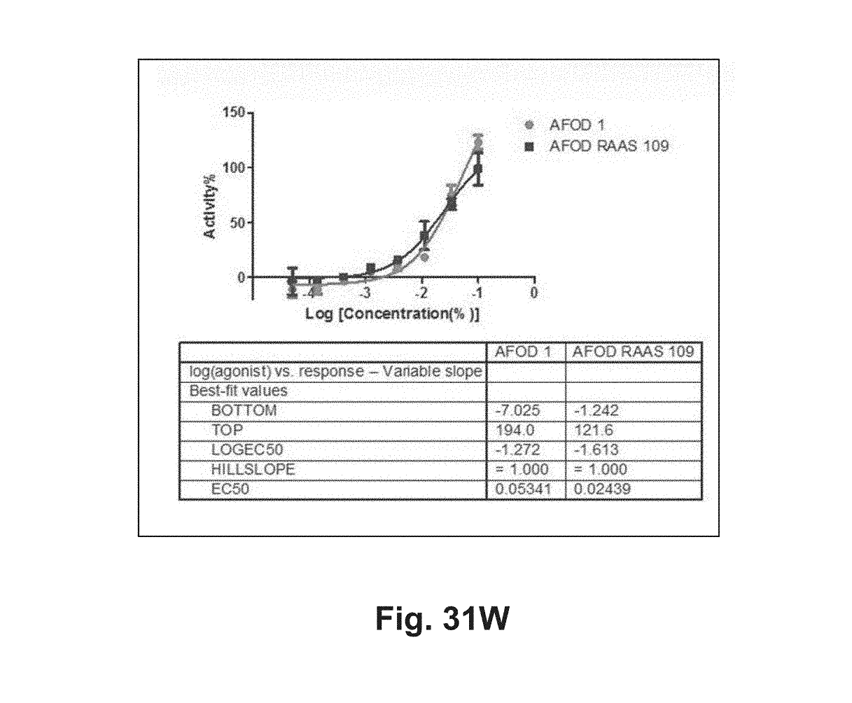

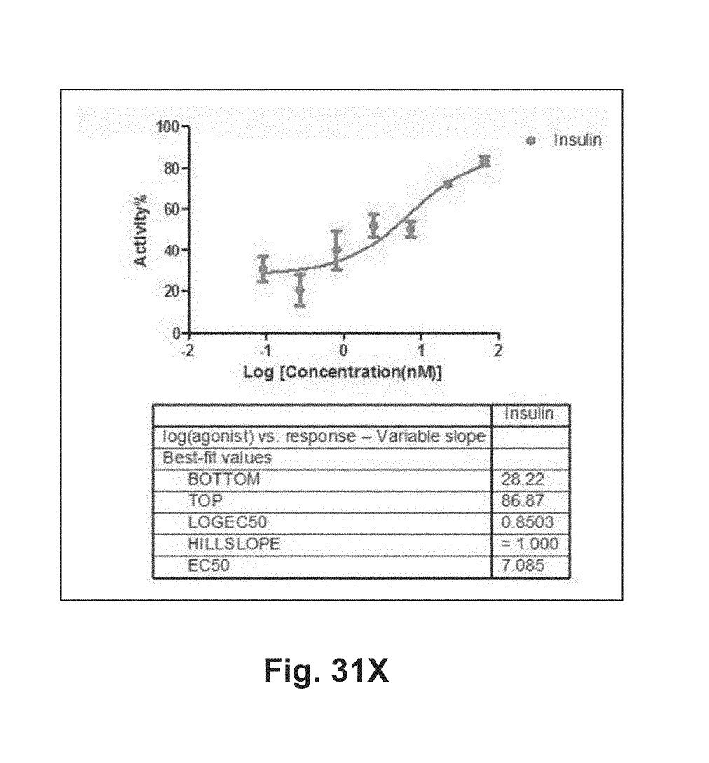

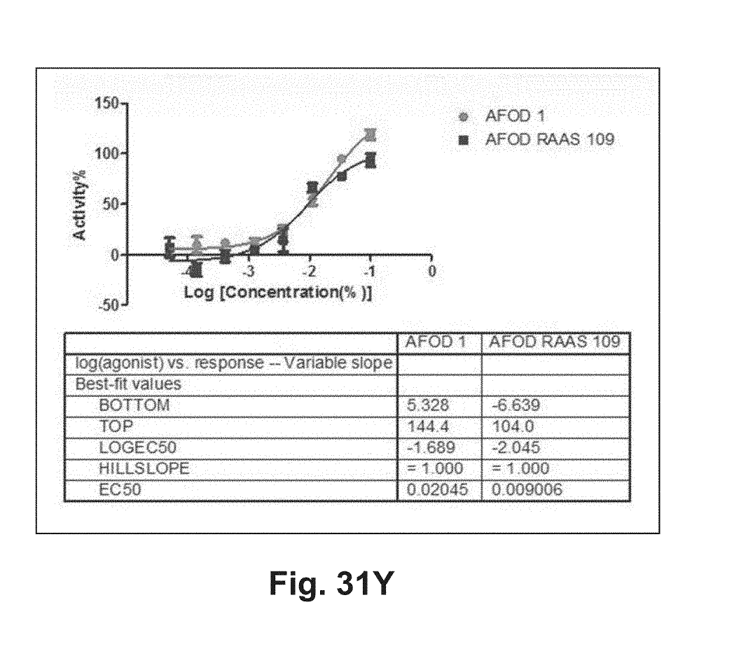

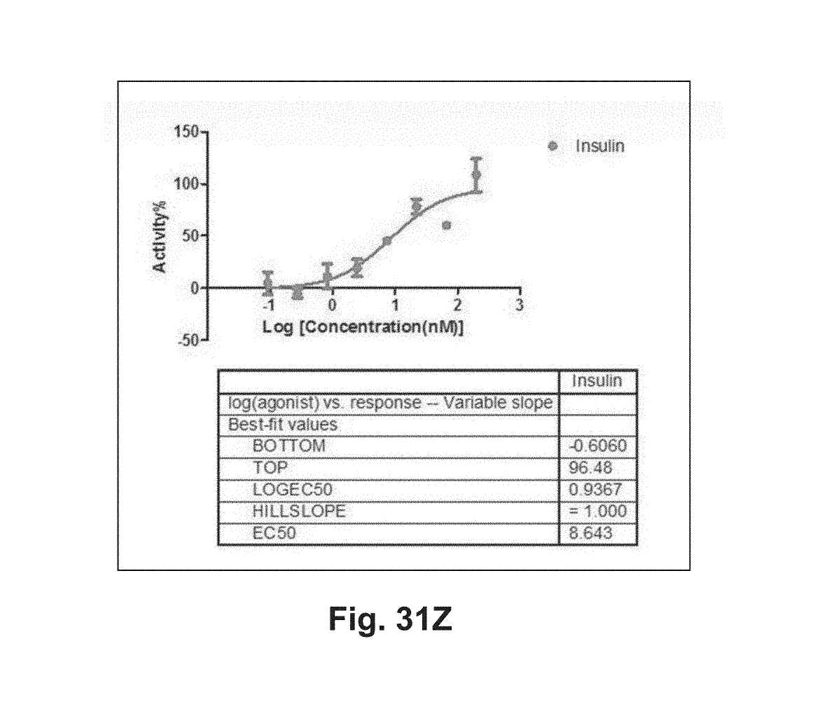

FIG. 31. Comparison with human NK cells on FACS

Conclusion

The characterization of unknown samples was carried out by staining with different cell surface markers for distinct cell lineages. Normal human peripheral blood cells were used as controls.

Vi-CELL cell viability analysis showed that 30 samples out of 59 samples had cells. Among these, only 10 samples had total cell number above 1×10⁵ and only 5 samples reached viability above 90% (Table 1).

FACS analysis indicated that the test samples may not contain any of the typical cells present in human peripheral blood.

Bioactivity Determination of Protein Samples in Glucose Uptake Assay

Outline
1. Study protocol
2. Data summary
3. Results
4. Conclusions

Study Protocol
1. 3T3-L1 fibroblasts were cultured in DMEM containing 25 mM glucose and 10% bovine calf serum at 37□ with 5% CO2.
2. 3T3-L1 fibroblasts were differentiated into adipocytes 2 days post confluent with the same DMEM medium containing 1 ug/ml insulin (Sigma-Aldrich), 1 uM dexamethasone (Sigma-Aldrich), and 0.5 mM isobutyl-1-methylxanthine (Sigma-Aldrich).
3. Media were replaced with DMEM containing 10% FBS and 1 ug/ml insulin and culture 2 days.
4. Seed 1×105 3T3-L1 adipocytes to 96 wells cell culture plate (DMEM, 10% FBS, 1% PS, 1 ug/ml insulin)
5. Before experiments, adipocytes were incubated in serum free medium for starvation overnight.
6. Dilute each sample 100 folds (2 ul primal solution to 198 ul buffer) in corresponding buffer. Add 2 ul of sample primal solution, 100 folds diluted solution and buffer to 198 ul KRPH buffer.
7. 3T3-L1 adipocytes were washed with KRPH buffer (5 mM Na2HPO4, 20 mM HEPES, pH 7.4, 1 mM MgSO4.1 mM CaCl2, 136 mM NaCl, 47 mM KCl, and 1% BSA) three times, Add 90 ul/well KRPH containing human insulin and samples to assay plate, incubate for 30 min at 37□ and 5% CO2.
8. Add 10 ul KRPH containing 0.25 uCi 1-[3H]-2-deoxyglucose/well and 50 umol/l 2-deoxyglucose and Incubate 10 min in 95% air/5% CO2 at 37□
9. The transport was stopped by rinsing the cells with cold PBS containing 10 mM glucose for three times.
10. The adipocytes were lysed in 50 ul 10% KOH for 5 min
11. Then the aliquots were subjected to scintillation counting using TriCap.

Data Summary

| | % Activation | | | | % Activation | | |
|---|---|---|---|---|---|---|---|
| Sample ID | First | Second | Average | Sample ID | First | Second | Average |
| AFOD 1 (0.1%) | 62.1 | 87.3 | 74.7 | KH 1 (1:100) | 15.9 | 20.89 | 18.4 |
| AFOD 1 (0.001%) | 9.6 | 28.9 | 19.2 | KH 1 (1:10000) | 13.02 | 23.11 | 18.1 |
| AFOD RAAS | 30.4 | 36.03 | 33.2 | KH 105 (1) | 18.2 | 9.66 | 13.9 |
| 103 (0.05%) | | | | (1:100) | | | |
| AFOD RAAS | 10.37 | 3.66 | 7.0 | KH 105 (1) | 1.73 | 1.7 | 1.7 |
| 103 (0.0005%) | | | | (1:10000) | | | |
| AFOD RAAS | 42.4 | 62.66 | 52.5 | KH 105 (2) | 27.76 | 41.78 | 34.8 |
| 107 (0.05%) | | | | (1:100) | | | |
| AFOD RAAS | 11.87 | 3.26 | 7.6 | KH 105 (2) | 2.07 | 11.88 | 7.0 |
| 107 (0.0005%) | | | | (1:10000) | | | |
| AFOD RAAS | 23.16 | 37.37 | 30.3 | AFOD RAAS | 3.54 | 2.97 | 3.3 |
| 108 (0.05%) | | | | 101 (0.1%) | | | |
| AFOD RAAS | 4.15 | 11.49 | 7.8 | AFOD RAAS | 17.71 | −13.19 | 2.3 |
| 108 (0.0005%) | | | | 101 (0.001%) | | | |
| AFOD RAAS | 53.8 | 72.19 | 63.0 | AFOD RAAS | −6.23 | 0.27 | -3.0 |
| 109 (0.1%) | | | | 121 (0.1%) | | | |
| AFOD RAAS | 13.02 | 4.44 | 8.7 | AFOD RAAS | 1.98 | −1.35 | 0.3 |
| 109 (0.001%) | | | | 121 (0.001%) | | | |
| AFOD RAAS | 10.71 | 15.54 | 13.1 | AFOD KH | 44.33 | 50.07 | 47.2 |
| 110 (0.05%) | | | | (0.1%) | | | |
| AFOD RAAS | 12.9 | 4.44 | 8.7 | AFOD KH | 20.68 | 11.07 | 15.9 |
| 110 (0.0005%) | | | | (0.001%) | | | |
| AFOD RAAS | 15.21 | 22.98 | 19.1 | | | | |
| 120 (0.05%) | | | | | | | |
| AFOD RAAS | 11.41 | 8.75 | 10.1 | | | | |
| 120 (0.0005%) | | | | | | | |

The results we obtained in two separated experiments are consistent. Sample AFOD 1, AFOD RAAS 107, AFOD RAAS 109 and AFOD KH show some potency in glucose uptake assay.

FIG. 31A—AFOD 1 results

FIG. 31B—AFOD RAAS 101 results

FIG. 31C—AFOD RAAS 103 results

FIG. 31D—AFOD RAAS 107 results

FIG. 31E—AFOD RAAS 108 results

FIG. 31F—AFOD RAAS 109 results

FIG. 31G—AFOD RAAS 110 results

FIG. 31H—AFOD RAAS 120 results

FIG. 31I—AFOD RAAS 121 results

FIG. 31J—AFOD KH results

FIG. 31K—Kieu Hoang AFCC KH1 results

FIG. 31L—KH 105 (1) results

FIG. 31K—KH 105 (2) results

Conclusions
1. The results we obtained in two times are consistent.
2. Sample AFOD 1, AFOD RAAS 107, AFOD RAAS 109 and AFOD KH show some potency in glucose uptake assay.

Bioactivity Determination of Protein Samples in Glucose Uptake Assay

Outline
1. Study protocol
2. Data summary for dose response assay
3. Results for dose response assay

Study Protocol
1. 3T3-L1 fibroblasts were cultured in DMEM containing 25 mM glucose and 10% bovine calf serum at 37□ with 5% CO2.
2. 3T3-L1 fibroblasts were differentiated into adipocytes 2 days post confluent with the same DMEM medium containing 1 ug/ml insulin (Sigma-Aldrich), 1 uM dexamethasone (Sigma-Aldrich), and 0.5 mM isobutyl-1-methylxanthine (Sigma-Aldrich).
3. Media were replaced with DMEM containing 10% FBS and 1 ug/ml insulin and culture 2 days.
4. Seed 1×105 3T3-L1 adipocytes to 96 wells cell culture plate (DMEM, 10% FBS, 1% PS, 1 ug/ml insulin)
5. Before experiments, adipocytes were incubated in serum free medium for starvation overnight.
6. Dilute each sample 100 folds (2 ul primal solution to 198 ul buffer) in corresponding buffer. Add 2 ul of sample primal solution, 100 folds diluted solution and buffer to 198 ul KRPH buffer.
7. 3T3-L1 adipocytes were washed with KRPH buffer (5 mM Na2HPO4, 20 mM HEPES, pH 7.4, 1 mM MgSO4.1 mM CaCl2, 136 mM NaCl, 47 mM KCl, and 1% BSA) three times, Add 90 ul/well KRPH containing human insulin and samples to assay plate, incubate for 30 min at 37□ and 5% CO2.
8. Add 10 ul KRPH containing 0.25 uCi 1-[3H]-2-deoxyglucose/well and 50 umol/l 2-deoxyglucose and Incubate 10 min in 95% air/5% CO2 at 37□
9. The transport was stopped by rinsing the cells with cold PBS containing 10 mM glucose for three times.
10. The adipocytes were lysed in 50 ul 10% KOH for 5 min
11. Then the aliquots were subjected to scintillation counting using TriCap.

Data Summary for Dose Response Assay

| | EC50 | | |
|---|---|---|---|
| | AFOD RAAS 107 | AFOD KH | insulin (nM) |
| N1 | No fit | 0.00596% | 3.09 |
| N2 | No fit | 0.00108% | 10.16 |
| N3 | No fit | 0.00087% | 4.173 |

The difference of EC50 is out of 3 folds between N1 and N2, so we run the N3. The results N2 and N3 are consistent.

For the sample AFOD RAAS 107 we found floccules in the solution

FIG. 31N—Dose response of AFOD RAAS 107 and AFOD KH_N1

FIG. 31O—Dose response of AFOD RAAS 107 and AFOD KH_N2

FIG. 31P—Dose response of AFOD RAAS 107 and AFOD KH_N3

Bioactivity Determination of Protein Samples in Glucose Uptake Assay

Outline
1. Study protocol
2. Data summary for dose response assay
3. Results for dose response assay
4. Conclusions and plan for dose response assay

Study Protocol
1. 3T3-L1 fibroblasts were cultured in DMEM containing 25 mM glucose and 10% bovine calf serum at 37□ with 5% CO2.
2. 3T3-L1 fibroblasts were differentiated into adipocytes 2 days post confluent with the same DMEM medium containing 1 ug/ml insulin (Sigma-Aldrich), 1 uM dexamethasone (Sigma-Aldrich), and 0.5 mM isobutyl-1-methylxanthine (Sigma-Aldrich).
3. Media were replaced with DMEM containing 10% FBS and 1 ug/ml insulin and culture 2 days.
4. Seed 1×105 3T3-L1 adipocytes to 96 wells cell culture plate (DMEM, 10% FBS, 1% PS, 1 ug/ml insulin)
5. Before experiments, adipocytes were incubated in serum free medium for starvation overnight.
6. Dilute each sample 100 folds (2 ul primal solution to 198 ul buffer) in corresponding buffer. Add 2 ul of sample primal solution, 100 folds diluted solution and buffer to 198 ul KRPH buffer.
7. 3T3-L1 adipocytes were washed with KRPH buffer (5 mM Na2HPO4, 20 mM HEPES, pH 7.4, 1 mM MgSO4.1 mM CaCl2, 136 mM NaCl, 47 mM KCl, and 1% BSA) three times, Add 90 ul/well KRPH containing human insulin and samples to assay plate, incubate for 30 min at 37□ and 5% CO2.
8. Add 10 ul KRPH containing 0.25 uCi 1-[3H]-2-deoxyglucose/well and 50 umol/l 2-deoxyglucose and Incubate 10 min in 95% air/5% CO2 at 37□
9. The transport was stopped by rinsing the cells with cold PBS containing 10 mM glucose for three times.
10. The adipocytes were lysed in 50 ul 10% KOH for 5 min
11. Then the aliquots were subjected to scintillation counting using TriCap.

Data Summary for Dose Response Assay

| | EC50 | | |
|---|---|---|---|
| | AFOD 1 | AFOD RAAS 109 | insulin (nM) |
| N1 | 0.0534% | 0.0244% | 7.085 |
| N2 | 0.0205% | 0.0090% | 8.643 |

FIG. 31Q—Dose response of AFOD 1 and AFOD RAAS 109_N1

FIG. 31R—Dose response of AFOD 1 and AFOD RAAS 109_N1

Conclusions
1. We have tested the sample AFOD 1 and AFOD RAAS 109 in dose response assay. The results we obtained in two times are consistent.

In Vivo Studies

The Study of APOAI Protein in Preventing Atherosclerosis and Related Cardiovascular Diseases

The current study was designed to investigate the human serum APOAI protein in preventing the atherosclerosis. New Zealand rabbits were adopted in this animal study and divided into 5 groups. They were high dose, medium dose and low dose of treatment, positive and vehicle control. The treatment groups were given APOAI via auricular vein once a week. Vehicle controls received normal saline via auricular vein once a week. Positive controls were given Liptor daily by p.o. with a dose of 0.45 mg/kg body weight. The body weight of animal was determined every week and whole blood was drawn every three weeks. The study duration was 19 weeks. At the end of study, all animals were sacrificed. The important organs like liver, heart, kidney, aorta, and arteria carotis were observed in gross and pathological sections. Lipid content was examined in liver and aorta. And liver index was also determined. Results showed that there was no significant change in body weight. The HDL-C was significantly high in all treatment groups when compared with vehicle control. Although the liver index was lower in treatment group, but there's no statistical difference found. The area of atherosclerosis was significant less in medium group when compared with vehicle control. The pathological examination showed that there was no calcification found in either vehicle control or treatment group. However there was one animal with calcification in positive control group. The pathological change of aorta was better in medium group when considering endothelium swelling, smooth muscle migrating and foam cell formation compared with vehicle control. But there is no significant improvement in low dose group. The cellular swelling and fat degeneration was better in the liver of medium than that of vehicle control. Although the cellular swelling was same in low dose group and vehicle control, but the fat degeneration was better in liver of low dose group than that of vehicle control. The lipid content in aorta was lower in treatment groups than that in vehicle control but there was no statistical significance. The lipid content in liver showed that TG in low and high dose group was significantly lower than that in vehicle control. The TC, TG and LDL-C in medium group were significantly lower than those in vehicle control.

Purpose of the Experiments:

To investigate the human serum APOAI in preventing atherosclerosis and related cardiovascular diseases and provide experimental basis for clinical application.

Methods and Materials

1, Tested Reagent
Product name: human Apolipoprotein AI, injection
Produced By: Shanghai RAAS Blood Products Co. Ltd.
Lot number:
Size: 50 mg/mL
Appearance: colorless liquid
Positive control: Liptor

2. Animal

Strain: New Zealand white rabbit

Vendor: Shanghai JieSiJie Laboratory Animal Co., Ltd

Qualification number:

Sex: male

Body weight: 1.8-2.0 kg

3 High Fat Diet Recipe

1% cholesterol+99% normal diet, provide by Shanghai SiLaiKe Laboratory Animal Center

4 Experimental Design

4.1 Model

Male New Zealand white rabbits were used in this study. The body weight was between 1.8-2.0 kg. The animals were quarantined for 5-10 days with normal diet before study. Blood samples were taken 12 hour after fasting before study to determine the blood lipid parameters.

4.2 Group

Animals were randomly divided into 5 groups including vehicle control, high dose, medium dose, low dose and positive control group. Ten to 14 rabbits were in one group. Each rabbit was fed with 30 gram of high fat diet followed by 120 gram of normal diet with free access to water.

Housing condition: Ordinary Animal Lab with temperature of 24±2□ and humidity of 55%±10%.

4.3 Administration

First dose was given 1 week before high fat diet. The frequency of dosing was once a week. Dose was 80, 40, 20 mg/kg body weight respectively. Drug was given by intravenous injection via auricular vein with the volume of 5 mL.

Liptor was given by intragastric administration

5 Parameters Tested:

5.1 body weight: body weight of each rabbit was determined once a week.

5.2 blood lipid parameters: whole blood was drawn every three weeks Animals were subject to 12 hour fast before taking blood. Resulted blood samples were kept still for 2 hours and then spin with 4,000 rpm for 10 min. The upper layer of serum was then separated and examined for total cholesterol (TC), total triglyceride (TG), low density lipoprotein cholesterin (LDL-C), and high density lipoprotein cholesterin (HDL-C). Test reagents were purchased from Shanghai Rong Sheng Bio-pharmaceutical Co. Ltd.

5.3 Pathological examination

A: The atherosclerosis of aorta (plaque area %)

B: Liver index

C: Aorta, liver, heart, arteria carotis, kidney

Results

1 the Establishment of Animal Model

Animals were fed with high fed diet and treatment as described above. All blood lipid parameters significantly increased. There was no significant difference between vehicle control and treatment groups (data shown below). After 12 weeks of high fat diet, 1 animal in vehicle control or treatment group was sacrificed respectively. The liver of animal in vehicle control showed cream white in color and there was no atherosclerosis observed in aorta. There was no abnormal change in the liver and aorta of animal in treatment group. After 16 weeks of high fat diet, 1 animal of vehicle control was sacrificed and found about 20% of plaque on the inner surface of aortic arch Animal continued to be fed with high fat diet and treatment for 3 more weeks. After 19 weeks of high fat diet, all animals were sacrificed.

2 Animal Procedures and Tissue Sampling

All animals were anesthetized by 20% of ethyl carbamate and then sacrificed with air injection. Abdomen cavity was opened. Whole blood was taken from heart. Heart was harvested along with 7 cm of aorta. Then other organs like liver, kidney and arteria carotis were harvested.

Connective tissue was stripped from resulted organs or tissues followed by washing in normal saline for 3 times. Pictures were taken then.

Aorta was cut from aortic arch, opened longitudinally and taken picture. The aorta was dissected for 0.5 cm from aortic arch, split longitudinally and then kept in cryo-preservation tube for later lipid analysis. One piece of this sample was fixed in formalin for further pathological analysis.

The weight of liver was determined immediately. Two pieces of specimen were cut from hepatic lobe. One was kept in cryo-preservation tube for lipid analysis and another one was fixed in formalin for further pathological analysis.

One piece of kidney sample was taken from renal pelvis and fixed in formalin for further pathological analysis.

Arteria carotis was dissected, cleaned and fixed in Formalin for further pathological examination.

The Formalin solution was replaced by fresh one about 4 hours and sent to pathological department for pathological section.

3 Results

3.1 Change of Body Weight

The body weight of each animal was determined before high fat diet and once a week thereafter. The change of body weight in each group was shown in table 1.

TABLE 1
The change of body weight in different groups

| Group | Wk0 | Wk 19 | Increase | Increase |
|---|---|---|---|---|
| (animal number) | (kg) | (kg) | (kg) | (%) |
| Vehicle (n = 9) | 1.94 ± 0.231 | 3.23 ± 0.284 | 1.29 ± 0.361 | 66.5% |
| High dose (n = 8) | 1.68 ± 0.078 | 3.49 ± 0.221 | 1.81 ± 0.209 | 107.1% |
| Medium dose | 1.8 ± 0.22 | 2.99 ± 0.52 | 1.18 ± 0.286 | 65.5% |
| (n = 9) | | | | |
| Low dose (n = 12) | 2.1 ± 0.174 | 3.19 ± 0.278 | 1.09 ± 0.529 | 51.9% |

3.2 Plasma Lipid Parameters

Animals were fast for 12 hours before taking blood samples via auricular vein. Resulted blood samples were kept still for 2 hours. The upper layer of serum was then separated and examined for total cholesterol (TC), total triglyceride (TG), low density lipoprotein cholesterin (LDL-C), and high density lipoprotein cholesterin (HDL-C). Test reagents were purchased from Shanghai Rong Sheng Bio-pharmaceutical Co. Ltd.

TABLE 2
Change of total triglyceride (TG)

| Group | | | | |
|---|---|---|---|---|
| (animal | Wk 0 | Wk 19 | Increase | Increase |
| number) | (mmol/L) | (mmol/L) | (mmol/L) | (%) |
| Vehicle | 0.823 ± 0.294 | 1.864 ± 0.871 | 1.041 ± 0.933 | 126.5% |
| (n = 9) | | | | |
| Medium | 0.656 ± 0.191 | 2.144 ± 1.043 | 1.488 ± 0.988 | 226.8% |
| dose | | | | |
| (n = 9) | | | | |
| Low dose | 0.786 ± 0.229 | 1.267 ± 0.772 | 0.482 ± 0.839 | 61.3% |
| (n = 12) | | | | |

TABLE 3
Change of total cholesterol (TC)

| Group | Wk0 | Wk 19 | Increase | Increase |
|---|---|---|---|---|
| (animal number) | (mmol/L) | (mmol/L) | (mmol/L) | (%) |
| Control(n = 9) | 1.15 ± 0.23 | 8.049 ± 2.99 | 6.896 ± 3.03 | 598.3% |
| High dose (n = 8) | 1.59 ± 0.48 | 12.49 ± 2.81 | 10.90 ± 2.66 | 685.5% |
| Medium dose | 1.77 ± 0.783 | 10.28 ± 5.82 | 8.505 ± 5.37 | 453.0% |
| (n = 9) | | | | |
| Low dose (n = 12) | 1.06 ± 0.27 | 9.07 ± 4.92 | 8.01 ± 4.87 | 755.6% |

TABLE 4
Change of high density lipoprotein cholesterin (HDL-C)

| Group | Wk 0 | Wk 19 | Increase | Increase | |
|---|---|---|---|---|---|
| (animal number) | (mmol/L) | (mmol/L) | (mmol/L) | (%) | Sig |
| Control(n = 9) | 0.94 ± 0.262 | 3.527 ± 2.007 | 2.588 ± 1.918 | 275.3% | |
| High dose (n = 8) | 1.183 ± 0.149 | 4.993 ± 2.018 | 3.81 ± 2.025 | 322.1% | 0.035* |
| Mediumdose(n = 9) | 0.67 ± 0.207 | 4.343 ± 2.439 | 3.674 ± 2.413 | 548.4% | 0.02* |
| Low dose (n = 12) | 0.705 ± 0.246 | 3.744 ± 2.14 | 3.04 ± 2.019 | 431.2% | 0.028* |

P < 0.05

TABLE 5
Change of ligh density lipoprotein cholesterin (LDL-C)

| Group | | | | |
|---|---|---|---|---|
| (animal | Wk0 | Wk 19 | Increase | Increase |
| number) | (mmol/L) | (mmol/L) | (mmol/L) | (%) |
| Control | 0.872 ± 0.386 | 5.826 ± 2.909 | 4.954 ± 2.953 | 568.1% |
| (n = 9) | | | | |
| High dose | 0.92 ± 0.324 | 14.1 ± 4.188 | 13.18 ± 4.053 | 1432.6% |
| (n = 8) | | | | |
| Medium | 1.06 ± 0.298 | 6.357 ± 4.475 | 5.297 ± 4.373 | 499.7% |
| dose | | | | |
| (n = 9) | | | | |
| Low dose | 0.826 ± 0.279 | 7.298 ± 4.60 | 6.472 ± 4.468 | 783.5% |
| (n = 12) | | | | |

TABLE 6
Liver index

| Group | | | | |
|---|---|---|---|---|
| (animal | Body weight | Liver weight | Liver index | |
| number) | (kg) | (g) | (%) | Sig |
| Control | 3.083 ± 0.279 | 123.08 ± 22.31 | 3.984 ± 0.579 | |
| (n = 9) | | | | |
| High dose | 3.565 ± 0.205 | 151.69 ± 18.49 | 4.257 ± 0.482 | 0.26 |
| (n = 8) | | | | |
| Medium | 3.009 ± 0.554 | 112.006 ± 25.79 | 3.708 ± 0.391 | 0.267 |
| dose (n = 9) | | | | |
| Low dose | 3.3 ± 0.329 | 128.096 ± 20.43 | 3.886 ± 0.489 | 0.571 |
| (n = 12) | | | | |

3.3 Plaque Area of Aorta

The aorta was dissected and opened for 7.5 cm from aortic arch longitudinally. Pictures were taken and atherosclerosis changing was analyzed. The area of atherosclerosis was graded by clinical standard according to its area to whole area of dissected aorta, by which grade I was less than 25%, grade II was between 25% to 50%, grade III was between 50% to 75% and Grade IV was greater than 75%.

TABLE 7
atherosclerosis change in vehicle control group

| Animal number | Plaque area/aorta area | Grade |
|---|---|---|
| 5 | 8.62 | I |
| 6 | 16.67 | I |
| 7 | 37.5 | II |
| 9 | 39.47 | II |
| 11 | 1.67 | I |
| 12 | 10 | I |
| 17 | 92.86 | IV |
| 18 | 70.91 | II |
| 19 | 25.17 | II |

Grade I: 4 animals;
Grade II: 4 animals;
Grade III: 0 animal;
Grade IV: 1 animal

TABLE 8
atherosclerosis change in low dose group

| Animal number | Plaque area/aorta area | Grade |
|---|---|---|
| 31 | 10 | I |
| 32 | 26 | II |
| 36 | 1.92 | I |
| 37 | 76.79 | III |
| 38 | 11.11 | I |
| 39 | 2.88 | I |
| 40 | 6.67 | I |
| 41 | 2 | I |
| 42 | 92 | IV |
| 43 | 6.67 | I |
| 44 | 0.18 | I |
| 48 | 23.36 | I |

Grade I: 9 animals;
Grade II: 1 animal;
Grade III: 0 animal;
Grade IV: 2 animals.

Statistical Analysis of Low Dose Group: Mann-Whitney Test

| Grade 0 | | I | | | | | | I | | I | |
|---|---|---|---|---|---|---|---|---|---|---|---|
| Level 1 | I | | I | I | I | I | I | | I | | I |
| Theoretic | 1 | 2 | 3 | 4 | 5 | 6 | 7 | 8 | 9 | 10 | 11 |
| level | | | | | | | | | | | |
| Level | 7 | 7 | 7 | 7 | 7 | 7 | 7 | 7 | 7 | 7 | 7 |
| Level 0 | | 7 | | | | | | 7 | | 7 | |
| Level 1 | 7 | | 7 | 7 | 7 | 7 | 7 | | 7 | | 7 |
| Grade 0 | I | | II | | II | II | III | | | IV | |
| Level 1 | | I | | II | | | | III | IV | | |
| Theoretic | 12 | 13 | 14 | 15 | 16 | 17 | 18 | 19 | 20 | 21 | |
| level | | | | | | | | | | | |
| Level | 7 | 7 | 15.5 | 15.5 | 15.5 | 15.5 | 18.5 | 18.5 | 19.5 | 19.5 | |
| Level 0 | 7 | | 15.5 | | 15.5 | 15.5 | 18.5 | | | 19.5 | |
| Level 1 | | 7 | | 15.5 | | | | 18.5 | 19.5 | | |

Level sum in Vehicle control: 112.8
Level sum in low dose group: 116.5
T_{0.05} = 71 T > T_{0.05} no statistical difference

TABLE 9
atherosclerosis change in medium dose group

| Animal number | Plaque area/aorta area | Grade |
|---|---|---|
| 21 | 36.53 | II |
| 22 | 1.69 | I |
| 23 | 18.75 | I |
| 25 | 19.17 | I |
| 27 | 11.67 | I |
| 28 | 1.82 | I |
| 29 | 61.67 | II |
| 30 | 1.6 | I |

Grade I: 6 animals;
Grade II: 2 animals;
Grade III: 0 animal;
Grade IV: 0 animal.

Statistical Analysis of Low Dose Group: Mann-Whitney Test

| Grade 0 | | I | | | I | I | | I | |
|---|---|---|---|---|---|---|---|---|---|
| Level 2 | I | | I | I | | | I | | I |
| Theoretic | 1 | 2 | 3 | 4 | 5 | 6 | 7 | 8 | 9 |
| level | | | | | | | | | |
| Level | 5.5 | 5.5 | 5.5 | 5.5 | 5.5 | 5.5 | 5.5 | 5.5 | 5.5 |
| Level 0 | | 5.5 | | | 5.5 | 5.5 | | 5.5 | |
| Level 2 | 5.5 | | 5.5 | 5.5 | | | 5.5 | | 5.5 |
| Grade 0 | | II | | II | II | II | III | IV | |
| Level 2 | I | | II | | | | | | |
| Theoretic | 10 | 11 | 12 | 13 | 14 | 15 | 16 | 17 | |
| level | | | | | | | | | |
| Level | 5.5 | 13 | 13 | 13 | 13 | 13 | 16 | 17 | |
| Level 0 | | 13 | | 13 | 13 | 13 | 16 | 17 | |
| Level 2 | 5.5 | | 13 | | | | | | |

Level sum in Vehicle control: 112.8
Level sum in low dose group: 46
T_{0.05} = 51 T < T_{0.05} statistical difference

TABLE 10
atherosclerosis change in high dose group

| Animal number | Plaque area/aorta area | Grade |
|---|---|---|
| 50 | 62.5 | II |
| 51 | 100 | IV |
| 52 | 56.88 | II |
| 53 | 40.13 | II |
| 54 | 100 | IV |
| 55 | 27.19 | II |
| 60 | 68.03 | II |
| 62 | 95.00 | IV |

Grade I: 0 animal;
Grade II: 5 animals;
Grade III: 0 animal;
Grade IV: 3 animals.

3.4 Pathological Examination

3.4.1 Aorta

| | | | | | | |
|---|---|---|---|---|---|---|
| | | | | Endo- | Smooth | |
| Animal | Plaque | Plaque | | thelium | muscle | Foam |
| number | (gross) | (section) | calcification | swelling | migrating | cell |

Vehicle control

| | | | | | | |
|---|---|---|---|---|---|---|
| 5 | + | + | − | − | + | + |
| 6 | + | − | − | − | − | − |
| 7 | ++ | + | − | + | − | + |
| 9 | ++ | − | − | + | − | + |
| 11 | − | − | − | − | − | − |
| 12 | +− | − | − | − | − | − |
| 13 | − | − | − | − | − | − |
| 17 | +++ | ++ | − | ++ | + | ++ |
| 18 | +++ | + | − | ++ | − | + |
| 19 | ++ | + | − | + | − | − |

Medium dose group

| | | | | | | |
|---|---|---|---|---|---|---|
| 21 | ++ | + | − | + | − | + |
| 22 | − | − | − | − | − | − |
| 23 | + | − | − | − | − | − |
| 25 | + | − | | − | | − |
| 27 | − | − | − | − | − | − |
| 28 | − | − | − | − | − | − |
| 29 | +++ | − | − | + | − | − |
| 30 | − | − | − | − | − | − |

Low dose group

| | | | | | | |
|---|---|---|---|---|---|---|
| 31 | − | − | − | − | | − |
| 32 | ++ | +++ | − | +++ | + | +++ |
| 37 | ++ | + | − | ++ | − | + |
| 38 | − | − | − | − | − | − |
| 39 | − | − | − | − | − | − |
| 40 | − | − | − | − | − | − |
| 41 | − | − | − | − | − | − |
| 42 | +++ | ++ | − | +++ | ++ | ++ |
| 43 | + | + | − | − | + | + |
| 44 | − | − | − | − | − | − |
| 48 | − | − | − | − | − | − |

High dose group

| | | | | | | |
|---|---|---|---|---|---|---|
| 50 | ++ | − | − | | | + |
| 51 | ++ | | | | | ++ |
| 52 | ++ | ++ | | | ++ | ++ |
| 53 | + | − | − | − | − | − |
| 54 | ++ | − | − | | | + |
| 55 | + | | − | − | − | − |
| 60 | +++ | ++ | | | ++ | +++ |
| 62 | +++ | − | − | − | − | − |

Positive control

| | | | | | | |
|---|---|---|---|---|---|---|
| 65 | − | − | − | − | − | − |
| 66 | + | ++ | − | − | + | + |
| 68 | + | − | + | − | − | + |
| +2 | ++ | | | | | |
| +3 | + | | | | | |

The pathological change was better in medium group when considering endothelium swelling, smooth muscle migrating and foam cell formation compared with vehicle control. But there is no significant improvement in low dose group

3.4.2 Liver Gross and Pathological Examination

| | | | |
|---|---|---|---|
| Anima # | Observation (color, texture and size) | Swelling | Fatty change |

Vehicle control

| | | | |
|---|---|---|---|
| 5 | dark red, white in some area, soft, left > right | ++ | + |
| 6 | dark red, smooth, soft, left > right | + | + |
| 7 | Pink, soft, left > right | + | + |
| 9 | pink,, less soft, | + | − |
| 11 | Pink, smooth, soft | ++ | + |
| 12 | pink, rough | +++ | + |
| 13 | dark red, some area showed pink, smooth, | + | − |
| | soft | | |
| 17 | Pink, partial rough, less soft | + | − |
| 18 | Partial pink, smooth, soft | − | − |
| 19 | Partial pink, smooth, soft | − | − |

Medium dose group

| | | | |
|---|---|---|---|
| 21 | dark red, partial pink, soft, less smooth | + | + |
| 22 | | ++ | − |
| 23 | dark red smooth, soft, left > right | − | − |
| 25 | dark red, partial pink, soft, smooth | − | − |
| 27 | dark red, partial pink, soft smooth | + | − |
| 28 | | ++ | − |
| 29 | dark red, soft, smooth | − | − |
| 30 | dark red, soft, smooth | − | − |

Low dose group

| | | | |
|---|---|---|---|
| 31 | Partial pink, soft, less smooth | ++ | − |
| 32 | Pink, soft, less smooth | + | + |
| 36 | Partial yellow, rough, less soft | +++ | + |
| 37 | Partial white, less soft, smooth | − | − |
| 38 | | − | − |
| 39 | Pink-white color, rough, less smooth | ++ | − |
| 40 | Pink at Hepatic portal, soft, less smooth | + | − |
| 41 | dark red, soft, smooth | − | − |
| 42 | Partial pink, soft, smooth | + | − |
| 43 | dark red, soft, smooth | − | − |
| 44 | dark red, soft, smooth | + | − |
| 48 | dark red, soft, smooth | − | − |

High dose group

| | | | |
|---|---|---|---|
| 50 | Partial yellow, rough surface, less soft | ++ | ++ |
| 51 | Yellow, rough surface, less soft | ++ | ++ |
| 52 | dark red, partial pink, rough surface, soft | − | − |
| 53 | Pink, rough surface, less soft | +++ | − |
| 54 | Pink, rough surface, soft | ++ | − |
| 55 | dark red, partial pink, rough surface, soft | +++ | − |
| 60 | Partial yellow, rough surface, less soft | + | − |
| 62 | dark red, partial pink, rough surface, soft | ++ | − |

Positive control group

| | | | |
|---|---|---|---|
| 65 | Yellow, rough surface, less soft | ++ | − |
| 66 | Yellow-white color, rough surface, less soft | +++ | − |
| 68 | Pink-white color at hepatic portal, dark red at | − | − |
| | outskirt, rough texture, les soft | | |
| +2 | Yellow color at hepatic portal, white at outskirt, | +++ | |
| | rough texture, less soft | | |
| +3 | Yellow, rough texture, less soft | +++ | |

The cellular swelling and fat degeneration was better in the liver of medium than that of vehicle control. Although the cellular swelling was same in low dose group and vehicle control, but the fat degeneration was better in liver of low dose group than that of vehicle control.

3.4.3 Heart, Arteria Carotis and Kidney

| | Heart/Coronary | | Arteria carotis | | kidney | |
|---|---|---|---|---|---|---|
| | Lipid | | Lipid | | Perirenal | Pathological |
| Animal number | infiltration | plaque | infiltration | plaque | adipose capsule | change |
| 5 | — | — | — | — | Full, thick | — |
| 6 | — | — | — | — | Full, thin | — |
| 7 | — | — | — | — | Full, relatively | — |
| | | | | | thick | |
| 9 | — | — | — | — | Full, relatively | — |
| | | | | | thick | |
| 11 | — | — | — | — | Full, thin | — |
| 12 | — | — | — | — | Full, relatively | — |
| | | | | | thick | |
| 13 | — | — | — | — | Full, a little thick | — |
| 17 | — | — | — | — | Full, a little thick | — |
| 18 | — | — | — | — | Full, a little thick | — |
| 19 | — | — | — | — | Full, relatively | — |
| | | | | | thick | |

Medium dose group

| | | | | | | |
|---|---|---|---|---|---|---|
| 21 | — | — | — | — | Full, thin | — |
| 22 | — | — | — | — | | |
| 23 | — | — | — | — | Spots, thin | — |
| 25 | — | — | — | — | Full, very thin | — |
| 27 | — | — | — | — | Full, very thin | — |
| 29 | — | — | — | — | Full, very thin | — |
| 30 | — | — | — | — | Full, very thin | — |

Low dose group

| | | | | | | |
|---|---|---|---|---|---|---|
| 31 | — | — | — | — | Full, very thin | — |
| 32 | — | — | — | — | Full, very thin | — |
| 36 | — | — | — | — | Full, very thin | — |
| 37 | — | — | — | — | Full, thin | — |
| 38 | — | — | — | — | Full a little thick | — |
| 39 | — | — | — | — | Full a little thick | — |
| 40 | — | — | — | — | Full, relatively | — |
| | | | | | thick | |
| 41 | — | — | — | — | Full, a little thick | — |
| 42 | — | — | — | — | Full, relatively | — |
| | | | | | thick | |
| 43 | | | | | | |
| 44 | — | — | — | — | Full, very thin | — |

High dose group

| | | | | | | |
|---|---|---|---|---|---|---|
| 50 | — | — | — | — | Full relatively | — |
| | | | | | thick | |
| 51 | — | — | — | — | Full relatively | — |
| | | | | | thick | |
| 52 | — | — | — | — | Full relatively | — |
| | | | | | thin | |
| 53 | — | — | — | — | Full, relatively | — |
| | | | | | thin | |
| 54 | — | — | — | — | Full, relatively | — |
| | | | | | thick | |
| 55 | — | — | — | — | Full, relatively | — |
| | | | | | thin | |
| 60 | — | — | — | — | Full, relatively | — |
| | | | | | thin | |
| 62 | — | — | — | — | Full, relatively | — |
| | | | | | thin | |

Positive control group

| | |
|---|---|
| 65 | Less full, thin |
| 66 | Full, thin |
| 68 | Full, thin |
| +2 | Full, thin |
| +3 | Less full, thin |

There was no pathological change found in heart and kidney either in vehicle control or treatment groups. There was no atherosclerosis change found in Arteria carotis.

3.4.3 Lipid Content in Tissues

1) Lipid Content in Liver

| | Control | Low dose | Middle | High |
|---|---|---|---|---|
| TC | 3.056 ± 0.775 | 2.95 ± 0.809 | 2.214 ± 0.515 | 2.841 ± 0.298 |
| TG | 1.817 ± 0.446 | 1.369 ± 0.251 | 1.081 ± 0.31 | 1.3 ± 0.171 |
| HDL- | 0.712 ± 0.244 | 0.803 ± 0.236 | 0.815 ± 0.249 | 0.825 ± 0.129 |
| C | | | | |
| LDL- | 2.035 ± 0.328 | 1.857 ± 0.559 | 1.407 ± 0.418 | 2.302 ± 0.054 |
| C | | | | |

Statistics Analysis of Lipid Content in Liver

| | Low dose | Medium | High |
|---|---|---|---|
| TC | 0.775 | 0.022 | 0.564 |
| TG | 0.022 | 0.011 | 0.009 |
| HDL-C | 0.81 | 0.74 | 0.684 |
| LDL-C | 0.436 | 0.011 | 0.989 |

The lipid content in liver showed that TG in low and high dose group was significantly lower than that in vehicle control. The TC, TG and LDL-C in medium group were significantly lower than those in vehicle control.

2) Lipid Content in Aorta

| | Control | Low dose | Middle | High |
|---|---|---|---|---|
| TC | 0.331 ± 0.097 | 0.28 ± 0.047 | 0.332 ± 0.135 | 0.29 ± 0.098 |
| TG | 0.406 ± 0.178 | 0.337 ± 0.055 | 0.388 ± 0.124 | 0.402 ± 0.101 |
| HDL- | 0.065 ± 0.032 | 0.092 ± 0.066 | 0.128 ± 0.064 | 0.111 ± 0.057 |
| C | | | | |
| LDL- | 0.323 ± 0.116 | 0.254 ± 0.078 | 0.307 ± 0.043 | 0.318 ± 0.05 |
| C | | | | |

Statistics Analysis of Lipid Content in Aorta

| | Low dose | Medium | High |
|---|---|---|---|
| TC | 0.387 | 0.879 | 0.483 |
| TG | 0.341 | 0.80 | 0.952 |
| HDL-C | 0.416 | 0.065 | 0.171 |
| LDL-C | 0.138 | 0.73 | 0.912 |

The lipid content in aorta was lower in treatment groups than that in vehicle control but there was no statistical significance.

Summary:

This study was designed to investigate the prevention efficacy of APOAI in atherosclerosis. The test article was given along with high fat diet which caused no significant decrease in blood lipid parameters. However the treatment significantly increased the HDL-C level in all treated groups. There was no dose escalation effect found in three treatment groups upon anatomic, pathological and biochemistry examination. It has been showed that the atherosclerosis in medium dose group was significantly less than that in vehicle control. The pathological change was better in medium group when considering endothelium swelling, smooth muscle migrating and foam cell formation in aorta compared with vehicle control. But there is no significant improvement in low dose group. The cellular swelling and fat degeneration was better in the liver of medium than that of vehicle control. Although the cellular swelling was same in low dose group and vehicle control, but the fat degeneration was better in liver of low dose group than that of vehicle control. The lipid content in aorta was lower in treatment groups than that in vehicle control but there was no statistical significance. The lipid content in liver showed that TG in low and high dose group was significantly lower than that in vehicle control. The TC, TG and LDL-C in medium group were significantly lower than those in vehicle control.

Appendix 1: Pictures of Aorta

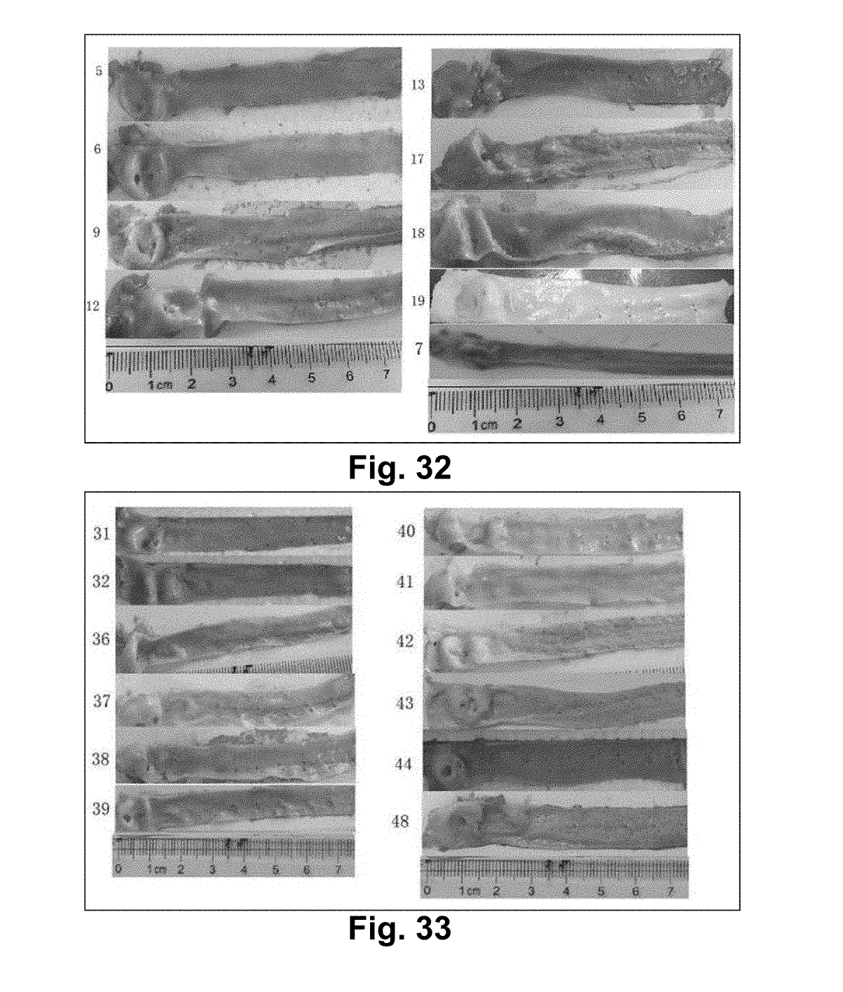

FIG. 32—Vehicle control

FIG. 33—Low dose group

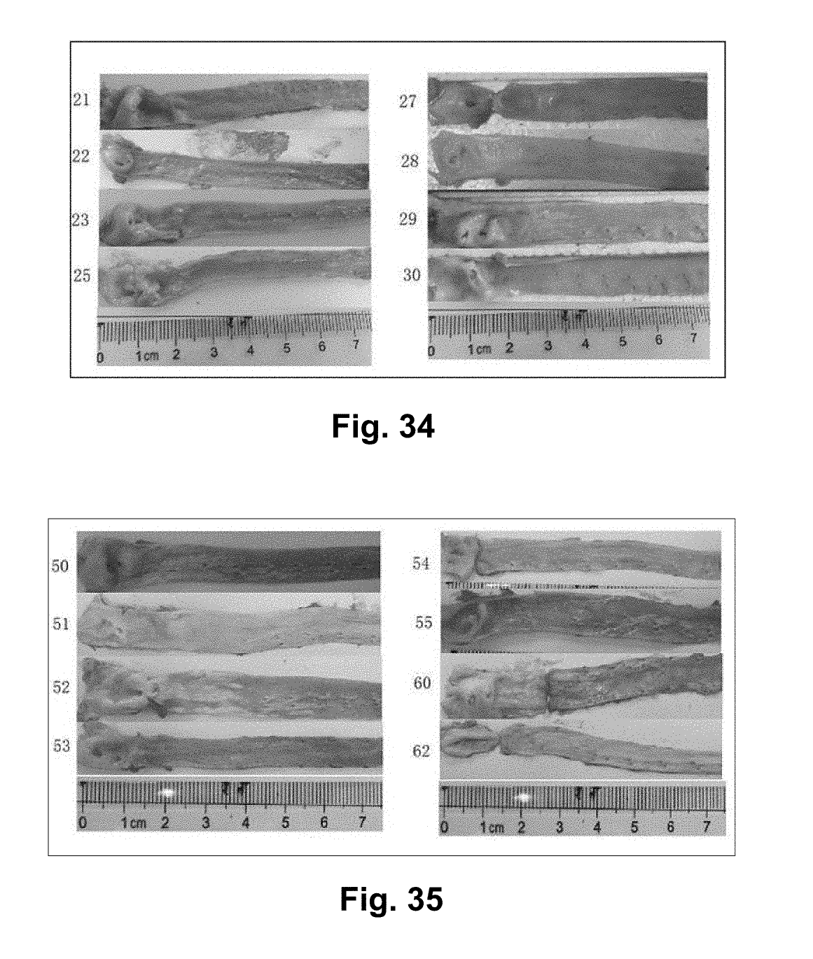

FIG. 34—Medium dose group

FIG. 35—High dose group

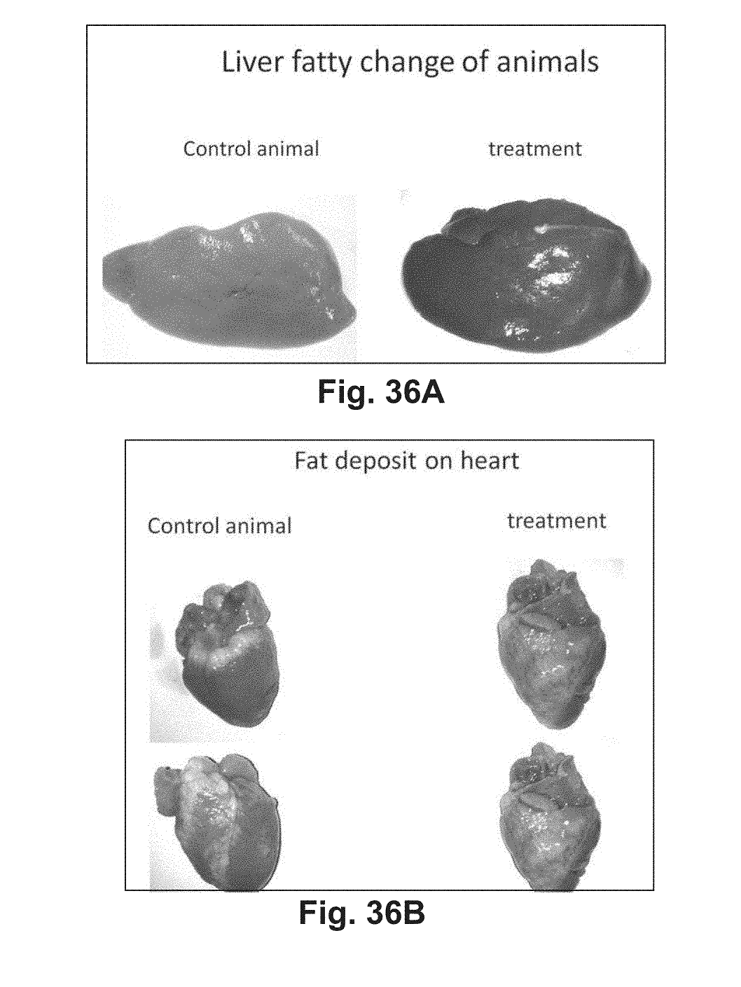

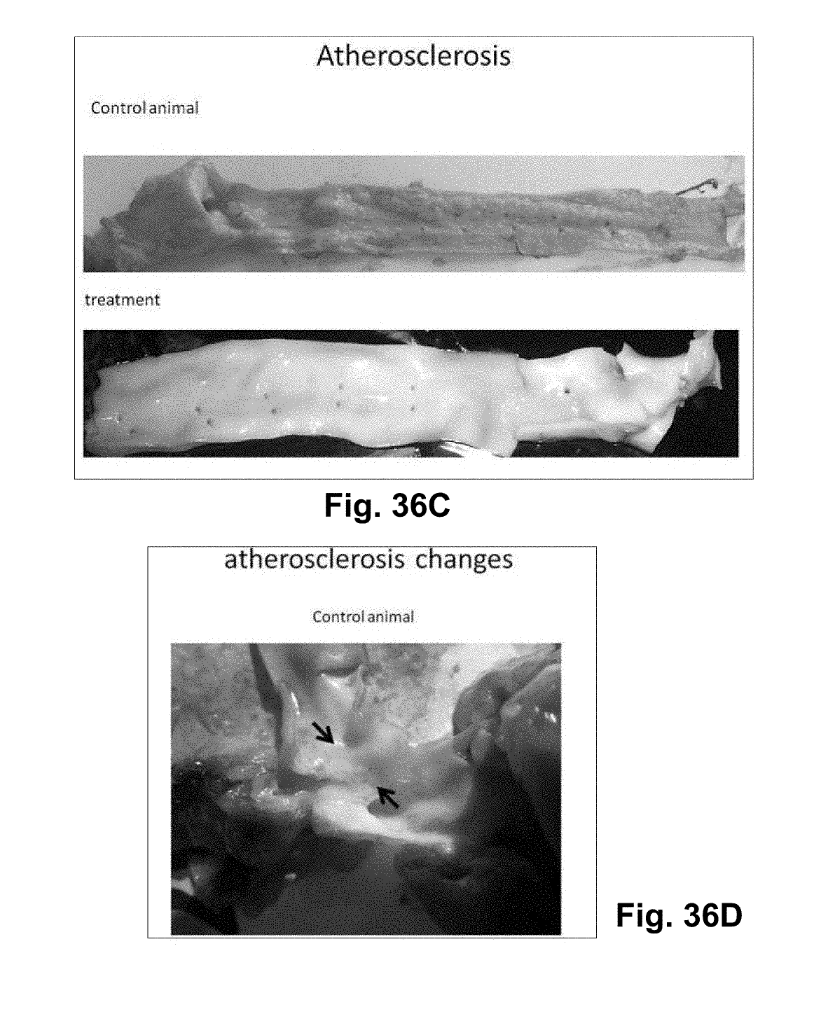

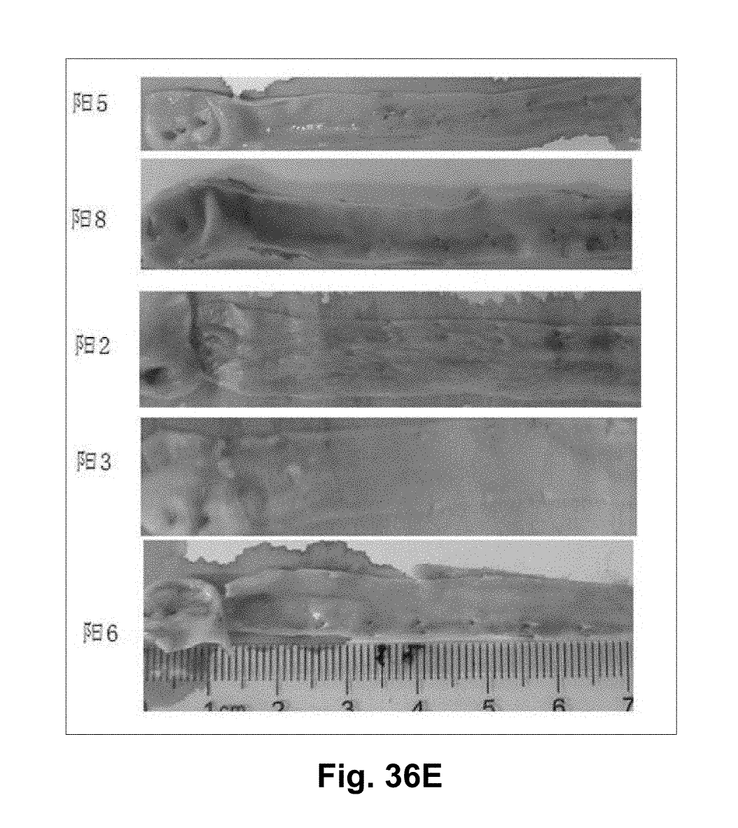

FIG. 36E—Positive control (Liptor)

FIG. 36A—Liver fatty change of control and treated animals

FIG. 36B—Fat deposit on heart of control and treated animals

FIG. 36C—Atherosclerosis on control and treated animals

FIG. 36D—Atherosclerosis change on control and treated animals.

Experimental Design and Results of Pilot Scale

Pre-Clinical Animal Test of Apo-AI

For the Antiatherogenic and Cholesterol-Lowing Properties

Apolipoprotein A-I (APOAI) is the major protein component of high density lipoprotein (HDL) in human plasma. The protein promotes cholesterol efflux from tissues to the liver for excretion and also helps to clear cholesterol from arteries Human APOAI protein was purified from pooled normal human plasma via chromatography with 98% of purity. Rabbit model with atherosclerosis was established in order to examine the efficacy of the resulted APOAI protein. APOAI was given intravenously to rabbits with dose escalation. Plasma lipid concentration was determined at indicated time point and the change of fatty streak lesions and liver tissue were also examined Our results showed that there was a decrease in all plasma lipid concentrations like total cholesterol (TC), tri-gliceride (TG), low density lipoprotein-cholesterol (LDL-C), very low density lipoprotein-cholesterol (VLDL-C), high density lipoprotein-cholesterol (HDL-C) in all animals received APOAI when compared to animal without APOAI treatment. There was also a significant change in fatty liver appearance. Although APOAI didn't stop the progress of fatty streak lesions, but it inhibited the growth of fatty streak lesions by 38% and 29% in two cohorts of animals respectively. Meanwhile the lipid content in aorta decreased in APOAI treated animals as well. Our results show that administration of APOAI can decrease the plasma lipid concentrations and inhibit the progress of fatty streak lesions in rabbits.

1. Purpose of the Experiments:

1.1 To establish an animal model of atherosclerosis

1.2 To investigate the efficacy of Apo-AI for the suppression of fatty streak lesions.

1.3 To investigate a dose escalation of APOAI in treating fatty streak lesions

2. Methods and Materials

2.1 Animal Procedures

Male New Zealand white-ear or other strain healthy rabbits (2.0 kg body weight, 4 in each group) were adopted. The rabbits were fed with normal diet under regular lab conditions for 5-10 days. The rabbits were fasted for 12 hrs before the beginning of the experiments. Blood parameters were then tested as the normal level of plasma indicators.

A total of 52 rabbits were purchased at different time, four of them were used as normal control and fed with normal diet the whole time during the experiments. There rest of the animals was switched to high fat diet for 10-11 weeks. When animal developed obvious fatty streak lesions in blood vessels, animals were randomly divided into 4 groups as following (FIG. 1).
1. Control group (without APOAI and Atorvastatin): n=4, animals were given normal diet after wk 10.
2. APOAI group 1: n=7, Apo-AI was administered once a week and 100 mg/each from wk 11 to wk 14; 50 mg/each were administered twice a week from wk 14 to wk 18.
3. APOAI group 2: n=7, Apo-AI was administered once a week and 100 mg/each from wk 11 to wk 14; 50 mg/each were administered twice a week from wk 14 to wk 18; 100 mg/each were administered once a week from wk 18 to wk 21.
4. Atorvastatin group: n=4, Atorvastatin was given to animal once a week from wk 10 to wk 14.

FIG. 1. Schematic Display of Animal Procedure.

2.2 Determination of Plasma Lipid Concentrations

Plasma concentrations of total cholesterol (TC), tri-gliceride (TG), low density lipoprotein-cholesterol (LDL-C), very low density lipoprotein-cholesterol (VLDL-C), high density lipoprotein-cholesterol (HDL-C) were quantified by enzymatic assays. TC/HDL-C or (LDL-C+VLDL-C)/HDL-C ratios were then determined.

2.3 Morphometric Evaluation of Atherosclerotic Lesions

Animals were sacrificed under deep anesthesia with sodium pentobarbital 25 mg/kg IV. The entire aorta from the aortic valve to the iliac bifurcation was removed from each rabbit and opened longitudinally. The vessel was fixed with 10% buffered formaldehyde (pH 7.4). Atheromatous lesions were measured without staining Atheromatous lesions were manually traced in the photographs, and the percent area of the atheromatous lesions was calculated.

2.4 Determination of Lipid Content in Dissected Aorta

Sample of aorta from animals were homogenated. And the content of lipid was determined.

3. Results

3.1 Establishment of Animal Model

At week 4, one of the lab animals were sacrificed and showed limited amount of fatty streak lesions. At week 10 and week 11, five lab animals were sacrifices and aortas were dissected. Obvious fatty streak lesions can be observed on the inner surface of the aorta. Fat deposit can also be observed on the liver tissues. During the animal model construction, 7 animals died during the first 4 weeks of high-fat diet due to stomach symptoms. Between week 7-10, 6 more lab animals died because of high-fat. The mortality rate is 16.7%. These animals were also dissected and 90% of them the aorta tissue showed fatty streak lesions occupied 20% of the total area (see FIG. 2).

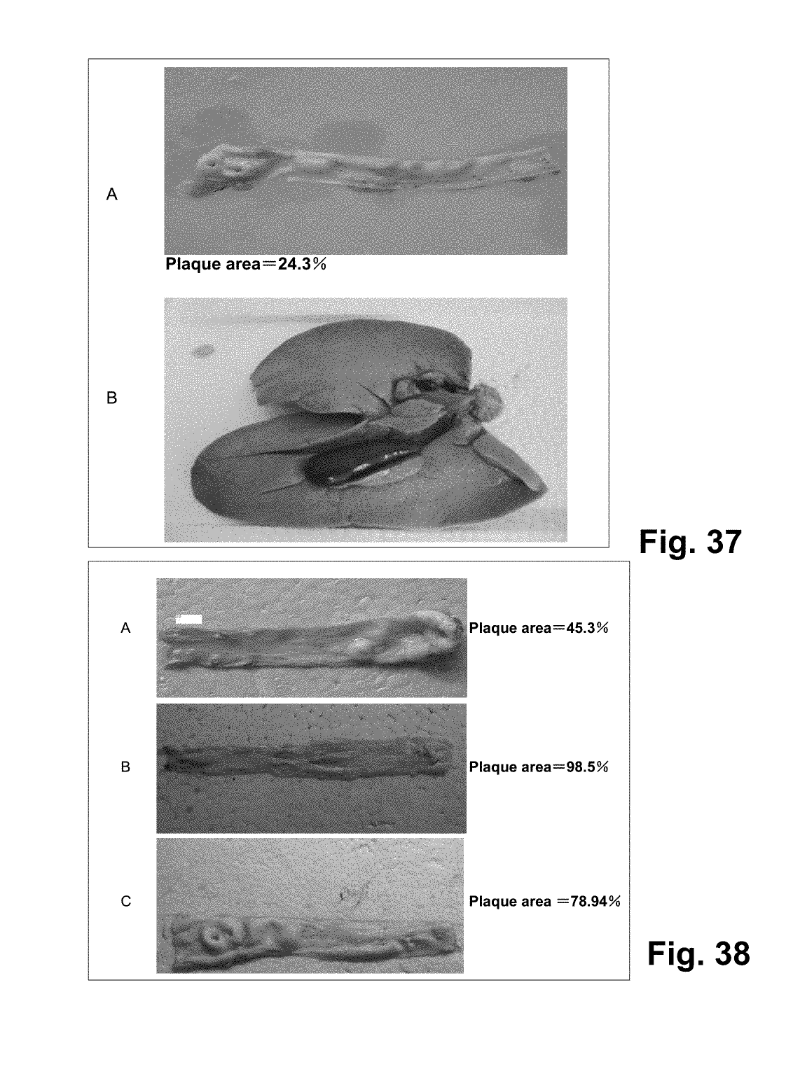

FIG. 37. The fatty streak lesions and liver change of animal fed with high fat diet and sacrificed on week 10. A) the lesions felt tender and hard as touched y hand. The fatty streak lesions of aorta were about 24.3% of the whole aorta. B) the surface of animal liver showed abnormal white colored spots which indicated a fat liver appearance

3.2 Successful Rate for Model Construction

During the animal model construction, 7 animals died during the first 4 weeks of high-fat diet due to stomach symptoms. Between week 7-10, 6 more lab animals died because of high-fat. The mortality rate is 16.7%. These lab animals were also dissected and 90% of them the aorta tissue showed fatty streak lesions occupied 20% of the total area (shown in FIG. 3).

FIG. 38. The plaque area change with normal diet after 10 weeks' high fat diet in control group. Control group were given normal diet after establishing fatty streak lesions in aortas (refer to methods and materials, animal procedure). A) the fatty streak lesions of aorta were about 45.3% of the whole aorta at Wk 14. B) and C) the fatty streak lesions of aorta were about 98.5% and 78.9.5% of the whole aorta in two animals at Wk 18.

3.3 Plasma Lipid Concentrations

1) APOA1 group1: The weight and plasma lipid concentrations of animals in APOA1 group 1 were determined at wk 0 (start of high fat diet), wk 10 (before APOA1 administration) and wk 18 (end of APOA1 administration).

| | | | | | | | TC/ |
|---|---|---|---|---|---|---|---|
| | Weight | TG | TCH | VLDL-C | HDL-C | LDL-C | HDL-C |
| Wk 0 | 2.164 | 0.967 | 1.152 | 0.870 | 0.748 | 0.282 | 1.938 |
| Wk 10 | 2.7 | 5.191 | 36.153 | 14.996 | 8.261 | 21.157 | 6.560 |
| Wk 18 | 2.79 | 1.17 | 3.69 | 1.09 | 1.46 | 2.60 | 3.000 |

2) APOA1 group 2: The weight and plasma lipid concentrations of animals in APOA1 group 2 were determined at wk 0 (start of high fat diet), wk 10 (before APOA1 administration) and wk 21 (end of APOA1 administration).

| | | | | | | | TC/ |
|---|---|---|---|---|---|---|---|
| | Weight | TG | TCH | VLDL-C | HDL-C | LDL-C | HDL-C |
| Wk 0 | 2.2 | 0.93 | 1.430 | 0.958 | 0.432 | 0.472 | 4.185 |
| Wk 10 | 2.45 | 4.507 | 34.683 | 15.443 | 10.168 | 19.24 | 3.667 |
| Wk 21 | 2.65 | 1.94 | 3.322 | 1.14 | 1.17 | 2.19 | 3.844 |

3) Atorvastatin group: The weight and plasma lipid concentrations of animals in Atorvastatin group were determined at wk 0 (start of high fat diet), wk 10 (before Atorvastatin administration) and wk 18 (end of Atorvastatin administration).

| | | | | | | | TC/ |
|---|---|---|---|---|---|---|---|
| | Weight | TG | TCH | VLDL-C | HDL-C | LDL-C | HDL-C |
| Wk 0 | 2.25 | 0.450 | 0.946 | 0.509 | 0.539 | 0.437 | 1.844 |
| Wk 10 | 2.85 | 9.122 | 20.339 | 9.710 | 8.404 | 10.911 | 4.511 |
| Wk 18 | 3.1 | 0.474 | 8.535 | 3.675 | 1.25 | 4.86 | 6.811 |

4) control group: The weight and plasma lipid concentrations of animals in control group were determined at wk 0 (start of high fat diet), wk 10 (before normal) and wk 18 (before sacrifice).

| | | | | | | | TC/ |
|---|---|---|---|---|---|---|---|
| | Weight | TG | TCH | VLDL-C | HDL-C | LDL-C | HDL-C |
| Wk 0 | 2.113 | 0.843 | 1.444 | 0.885 | 0.684 | 0.559 | 2.108 |
| Wk 10 | 2.742 | 2.666 | 32.42 | 7.467 | 5.657 | 24.953 | 9.459 |
| Wk 18 | 3.1 | 1.207 | 5.277 | 1.961 | 0.759 | 3.316 | 6.458 |

5) changes of plasma lipid concentrations in APOAI treated animals compared to control group animals (FIG. 4).

In APOAI group 1 and APOAI group 2, the main concentrations of plasma lipid decreased after 8 or 11 weeks of Apo-AI treatment meanwhile the decrease in control group was also observed. There is a significant decrease in VLDL-C and TC/HDL-C of APOAI group 1 when compared to control group (P<0.05). There is no significant change in the rest of values.

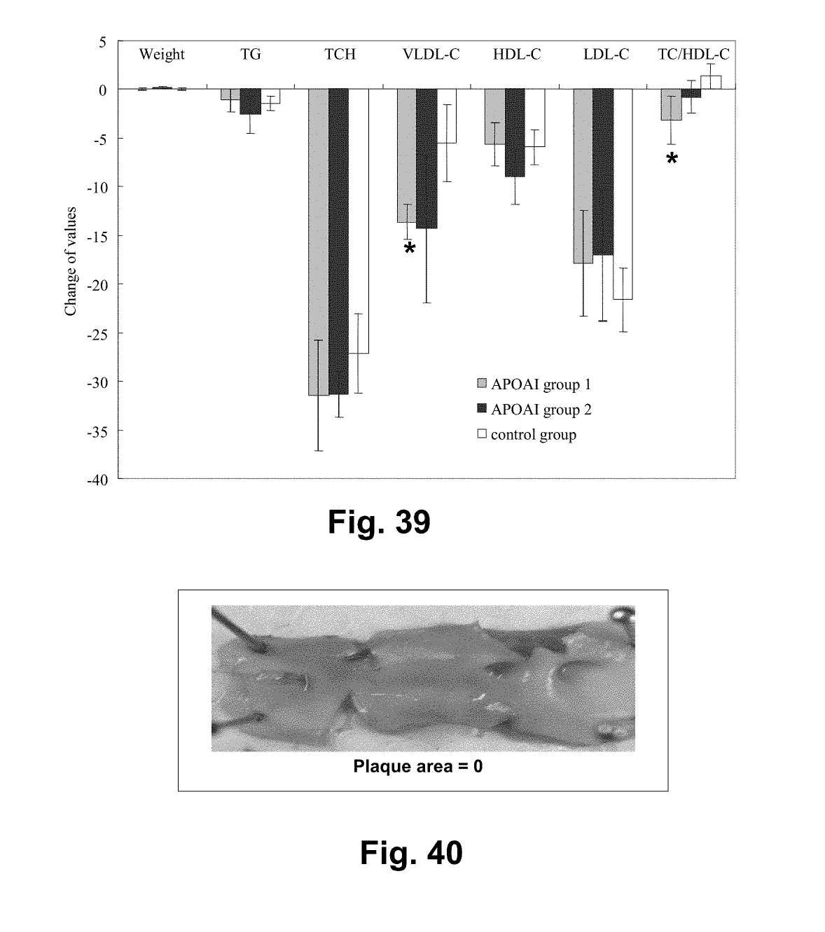

FIG. 39. The change of weight and plasma lipid concentrations were compared between APOAI treated and control animals. Data were obtained on wk 18, wk 21 and wk 18 from APOAI group 1 (gray solid column), APOAI group 2 (dark solid column) and control group (white column) respectively. X axis stands for the change of each value at the end of experiment compared to wk 10. Y axis stands for the parameters examined. * represents P<0.05

6) Changes of HDL-C in Animals

The change of HDL-C was determined between wk 0 and at the end of experiment (that is wk 18 in APOAI group 1 and control group and wk 21 in APOAI group 2) in APOAI treated and control animals Since HDL-C represents the lipid concentration carried by HDL which is formed by APOAI and phospholipids, so this result indicated that administration of Apo-AI could lower blood cholesterol through the formation of HDL.

| | | Wk 18 or wk | Concentration | |
|---|---|---|---|---|
| | Wk 0 | 21 | increased | Fold increased |
| APOAI Group 1 | 0.748 | 1.464 | 0.716 | 1.436 |
| APOAI Group 2 | 0.432 | 1.423 | 0.992 | 3.078 |
| Control group | 0.684 | 0.759 | 0.074 | 0.102 |

3.4 Change of Liver Tissue

1) Pathological Examination of Liver Tissue

The gross change of liver surface is white colored spots observed at wk 10 of establishing the animal model. The surface of the liver feels harder than normal tissue. Histological analysis showed fatty liver change. The liver samples taken from the APOAI treated group showed less fatty change in gross specimen and the surface is not as hard as that at wk 10. The control group also showed relief in its gross chance. The probable reason is that the high cholesterol and atherosclerosis model is established in a relative short period of time, switch to normal diet also helped to alleviate the symptoms.

2) Liver Index

There is no difference in liver index between APOAI treated and control group.

| | Weight (g) | Liver index |
|---|---|---|
| APOAI Group 1 | 0.09 | 0.033 |
| APOAI Group 2 | 0.117 | 0.044 |
| Control group | 0.111 | 0.036 |

3.5 Fatty Streak Lesions

The fatty streak lesions were examined in all animals at the end of the experiment. The area of the atherosclerosis was determined and then compared to wk 10 and wk 18 of control group respectively.

| | | Fatty | | | | |
|---|---|---|---|---|---|---|
| | | streak | Compare | | Compare | |
| | | lesions | to wk 10 | | to wk 18 | |
| | Time | area | of control | Increase | of control | Decrease |
| | point | (%) | group | % | group | % |
| APOAI | Wk 18 | 43.84 | 19.03 | 77 | −27.36 | 38.43 |
| Group 1 | | | | | | |
| APOAI | Wk 21 | 50.51 | 25.71 | 104 | −20.69 | 29.05 |
| Group 2 | | | | | | |
| Control | Wk 18 | 71.20 | 46.39 | 187 | | |
| group | | | | | | |
| Control | Wk 10 | 24.81 | | | | |
| group | | | | | | |

1) Gross examination of Fatty streak lesion: there was accumulation and swelling on aortic wall. The tissue was tender and hard when touched with hand. Dissection of the blood vessels showed fat deposit in the cross-section of the tissue. The fatty streak lesion decreases as the aorta desends. Compared with the control group, there were no bumps on the inner surface of aorta in APOAI treated group. The tissue feels soft.

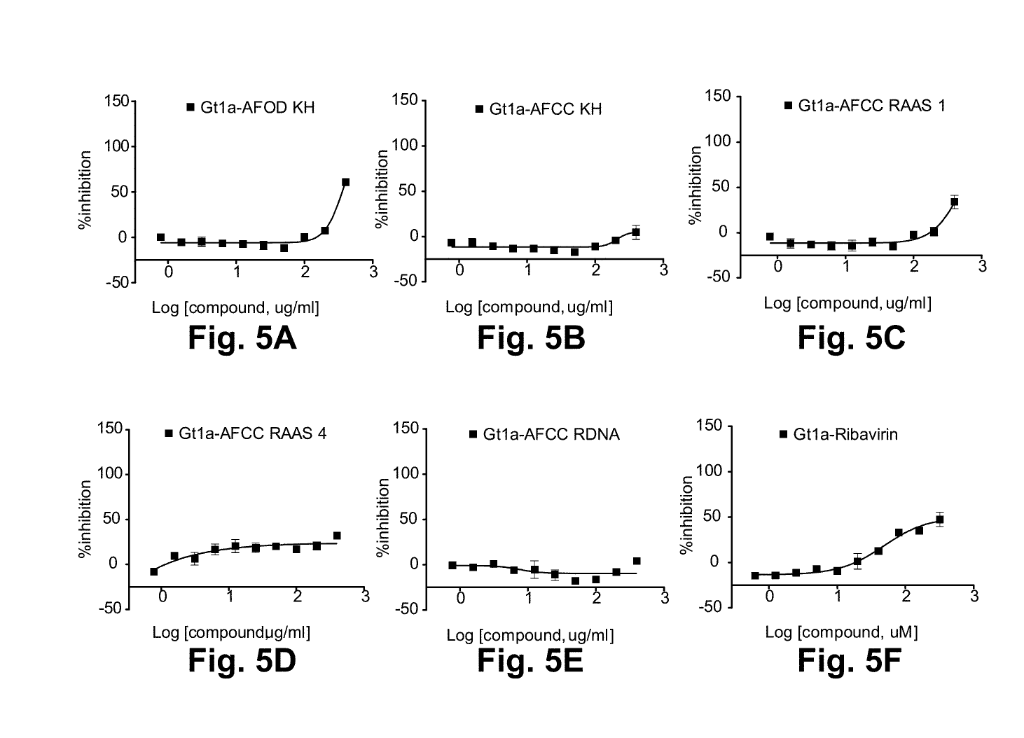

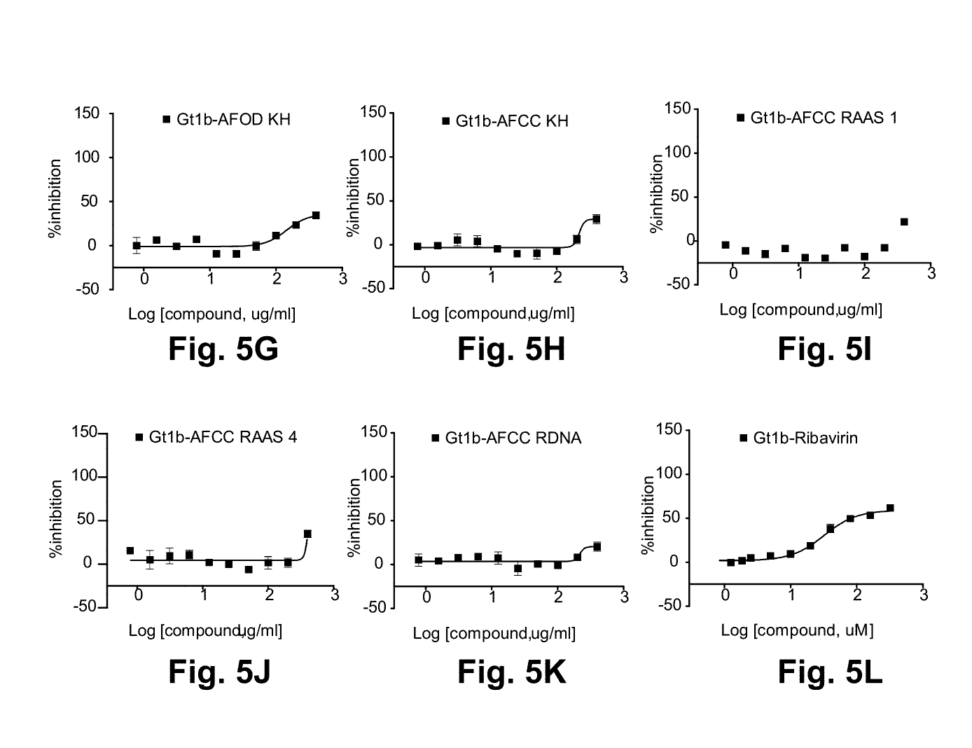

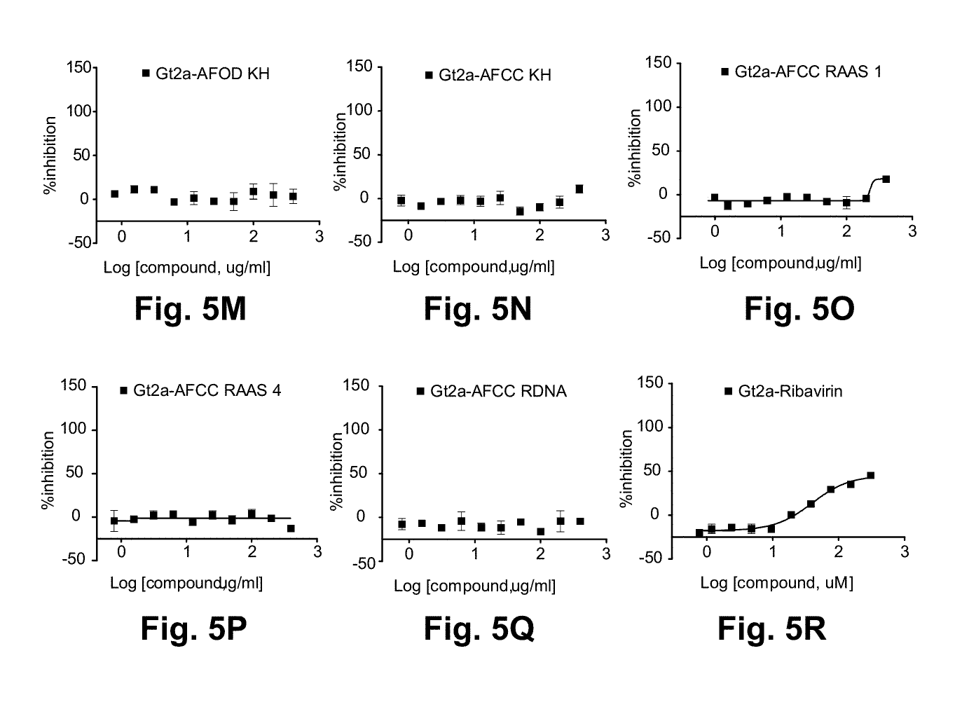

2) Area measurement of the fatty streak lesion: the area of the fatty streak lesion increased 77% or 104% in APOAI treated animals and 187% in control group. Compared to control group at wk 10, the area of fatty streak lesion decreased by 38.43% and 29.05% in APOAI group 1 and APOAI group 2 respectively (shown in FIGS. 5, 6, and 7).

FIG. 40. Normal rabbit aorta without fatty streak lesion.

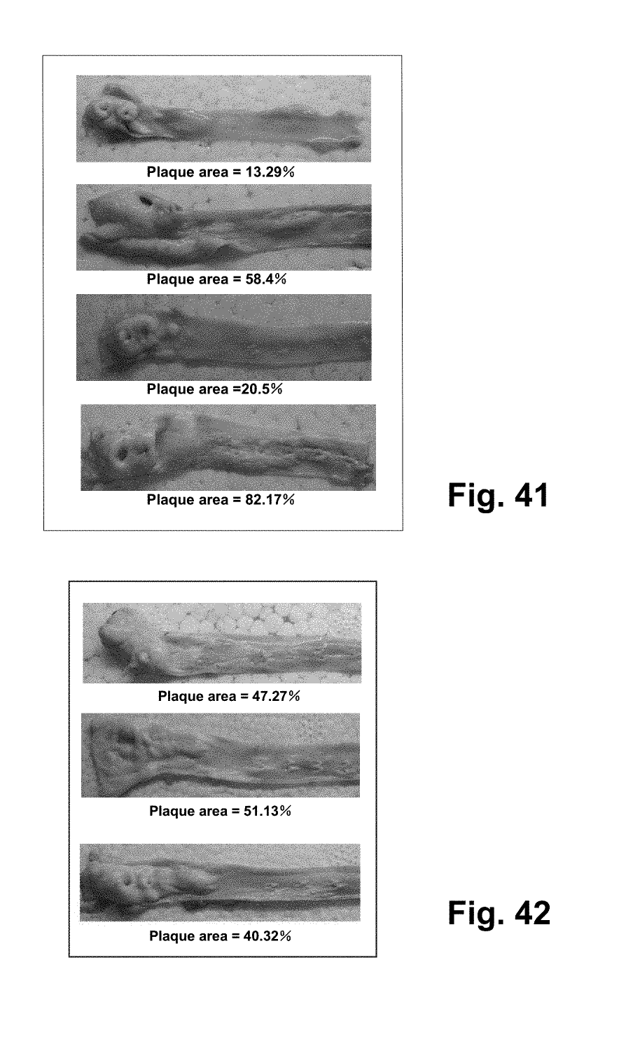

FIG. 41. the area of fatty streak lesion in aorta from APOAI group 1.

FIG. 42. the area of fatty streak lesion in aorta from APOAI group 2

3) Analysis of Lipid Content at Dissected Aorta

The lipid content in aorta was determined in all groups. The triglyceride content at dissected aorta of the APOAI group is significantly lower to that in control group (P<0.05).

| | | P value (compared to |
|---|---|---|
| | lipid con. (umol/mg) | control group) |
| APOAI Group 1 (n = 7) | 0.025 ± 0.0095 | 0.006 |
| APOAI Group 2 (n = 4) | 0.0267 ± 0.0054 | 0.015 |
| Atorvastatin group (n = 4) | 0.0274 ± 0.006 | 0.046 |
| Control group (n = 4) | 0.0736 ± 0.014 | |

4 Summary

The purpose of this preclinical animal is to test the dose and efficacy of APOAI in inhibiting the development of fatty streak lesion in rabbits.

Based on data from the experiments, it takes 4-5 weeks to establish a high cholesterol rabbit model need and 10 weeks to form atherosclerosis fatty streak lesion with high-fat diet (the average surface area of fatty streak lesion is 24% at week 10). 60% of animals will develop fatty streak lesion in aorta.

After intravenous infusion of human APOAI at various doses, the hypercholesterolemia and liver lesion improved dramatically, but APOAI inhibits rather than stop the progress of fatty streak lesion in aorta.

The experiment shows that the administration of APOAI to hypercholesterolemia lab animals reduces the surface area of fatty streak lesion in aorta and decreases the triglyceride content in the lesion tissue, thus, APOAI is a candidate of anti-atherogenic and anti-cholesterol medicine.

Report Title: Antiviral Efficacy of AFCC in an Influenza H1N1-Infected Mouse Model

Part 1 Pilot Study

Content

Objective

Infection with human influenza virus (IFV) causes respiratory tract illness in human and animals including mice. Mouse model infected Intranasally with IFV H1N1 is well recognized for anti-IFV compound screening. This study is designed to evaluate in vivo anti-IFV activity of a blood-derived product AFCC from RAAS in the mouse model and to identify appropriate dosages for the in vivo efficacy study.

Study Method

Study RAAS-20120216B was executed in the following steps:

1) Treat mice with RAAS blood product AFCC-KH.

1) Infect mice with IFV by intranasal inoculation.

2) Observe mice for 26 days.

3) Sacrifice mice in the end of the study.

Result Summary

In the pilot study all mice that were injected q.o.d. iv/ip in turn with AFCC survived through whole period of observation time (14 days) and their body weights varied in the normal range without significant loss. This indicates that the dosage and regimen of AFCC administration were well tolerated by the treated mice. Thereafter the 14-day-treated mice, together with an additional group of untreated mice as vehicle were challenged intranasally with IFN WSN. The AFCC treatment for 2 weeks before IFN WSN infection significantly decreased the mouse mortality and prolonged mouse survival time.

Report for RAAS-20120216B

I. Method

Animals:

Female BALB/c mice (6-8 weeks, 17-22 g) were divided into defined study groups after a visual examination and a 3 to 5-day acclimation upon arrival.

Solution Preparation:

1. Sodium Pentobarbital: Freshly dissolved in saline for injection at 8 mg/ml prior to using.

2. Test article: human plasma derived protein AFCC in sterile solutions for vein injection provided by the client.

Experimental Procedure:

IFV Infection and Test Article Administration:

1. From day 1 to day 14, AFCC KH 1 is intravenously and/or intraperitoneally administrated for 14 days.

2. On day 15, mice are anesthetized by intraperitoneal injection of sodium pentobarbital (80 mg/kg). Mice are inoculated with 5×10̂3 pfu of Influenza H1N1 A/WSN/33 via the intranasal route in SFM medium.

3. From day 1 through day 40 mice are observed two times a day. Mortality and body weight are recorded daily.

4. On day 40, the experiment is terminated by sacrificing survived mice.

II. Groups and Schedules:

TABLE 1
Action summary of Study WX-IFV02162012

| | | | IFV | AFCC, | |
|---|---|---|---|---|---|
| Study Day | Date | Weighing | challenge | iv/ip | mouse sacrifice |
| Day 1 | 02162012 | ✓ | | ✓ | |
| Day 2 | 02172012 | ✓ | | | |
| Day 3 | 02182012 | ✓ | | ✓ | |
| Day 4 | 02192012 | ✓ | | | |
| Day 5 | 02202012 | ✓ | | ✓ | |
| Day 6 | 02212012 | ✓ | | | |
| Day 7 | 02222012 | ✓ | | ✓ | |
| Day 8 | 02232012 | ✓ | | | |
| Day 9 | 02242012 | ✓ | | ✓ | |
| Day 10 | 02252012 | ✓ | | | |
| Day 11 | 02262012 | ✓ | | ✓ | |
| Day 12 | 02272012 | ✓ | | | |
| Day 13 | 02282012 | ✓ | | ✓ | |
| Day 14 | 02292012 | ✓ | | | |
| Day 15 | 03012012 | ✓ | ✓ | ✓ | |
| Day 16 | 03022012 | ✓ | | | |
| Day 17 | 03032012 | ✓ | | | |
| Day 18 | 03042012 | ✓ | | | |
| Day 19 | 03052012 | ✓ | | | |
| Day 20 | 03062012 | ✓ | | | |
| Day 21 | 03072012 | ✓ | | | |
| Day 22 | 03082012 | ✓ | | | |
| Day 23 | 03092012 | ✓ | | | |
| Day 24 | 03102012 | ✓ | | | |
| Day 25 | 03112012 | ✓ | | | |
| Day 26 | 03122012 | ✓ | | | |
| Day 27 | 03132012 | ✓ | | | |
| Day 28 | 03142012 | ✓ | | | |
| Day 29 | 03152012 | ✓ | | | |
| Day 30 | 03162012 | ✓ | | | |
| Day 31 | 03172012 | ✓ | | | |
| Day 32 | 03182012 | ✓ | | | |
| Day 33 | 03192012 | ✓ | | | |
| Day 34 | 03202012 | ✓ | | | |
| Day 35 | 03212012 | ✓ | | | |
| Day 36 | 03222012 | ✓ | | | |
| Day 37 | 03232012 | ✓ | | | |
| Day 38 | 03242012 | ✓ | | | |
| Day 39 | 03252012 | ✓ | | | |
| Day 40 | 03262012 | ✓ | | | ✓ |

✓ indicates that the action was taken.

TABLE 2
Experimental Design for the pilot experiment

| | | | |
|---|---|---|---|
| | AFCC-KH | | |
| Day | (ml/mouse) | animal number | H1N1 WSN |

| | | | | |
|---|---|---|---|---|
| 1 | iv, 0.2 | 5 | | |
| 3 | ip, 0.6 | 5 | | |
| 5 | iv, 0.2 | 5 | | |
| 7 | ip, 0.6 | 5 | | |
| 9 | iv, 0.2 | 5 | | |
| 11 | ip, 0.6 | 5 | | |
| 13 | iv, 0.2 | 5 | | |
| 15 | ip, 0.6* | 5 | 5 | in, 5 × 10\hat{ }3 |
| | | | | pfu/mouse |
| 17 | | 5 | 4 | |
| 19 | | 5 | 4 | |
| 21 | | 5 | 4 | |
| 23 | | 5 | 4 | |
| 25 | | 5 | 4 | |
| 27 | | 5 | 4 | |
| 29 | | 5 | 4 | |
| 31 | | 5 | 4 | |
| 33 | | 5 | 4 | |
| 35 | | 5 | 4 | |
| 37 | | 5 | 4 | |
| 39 | | 5 | 4 | |
| 40 | | 5 | 4 | |

*ip mice 4 h post H1N1 challenge.

III Adverse Events and Tolerability of Compounds:
1. In the AFCC treatment group, one mouse died of severe face and neck damages on Mar. 3, 2012 (experimental day 17) due to serious fight among mice. This mouse was eliminated for final data analysis.

Results and Discussion

To serve the purpose to identify appropriate dosages for the efficacy study a small scale pilot experiment was carried out. In the pilot study all mice that were injected q.o.d. iv/ip in turn with AFCC survived through whole period of observation time (14 days) and their body weights varied in the normal range without significant loss. This indicates that the dosage and regimen of AFCC administration were well tolerated by the treated mice.

Thereafter the 14-day-treated mice, together with an additional group of untreated mice as vehicle were challenged intranasally with IFN WSN. The result indicated that all 5 mice in the H1N1-challenged vehicle control group died (FIG. 2). In comparison to the vehicle group, 50% mice pre-treated with AFCC survived to the end of experiment (FIG. 2) and their body weights started to recover within 9-24 days post-IFV WSN challenge after severe body weight loss of first a few days post WSN challenge (FIG. 3, FIG. 4). Treatment with AFCC significantly prolonged the survival time of the other 50% pre-treated mice by 18 days, compared with the vehicle group although the AFCC treatment didn't prevent their body weight loss (FIG. 4). In conclusion the AFCC treatment for 2 weeks before IFN WSN infection significantly decreased the mouse mortality and prolonged mouse survival. time.

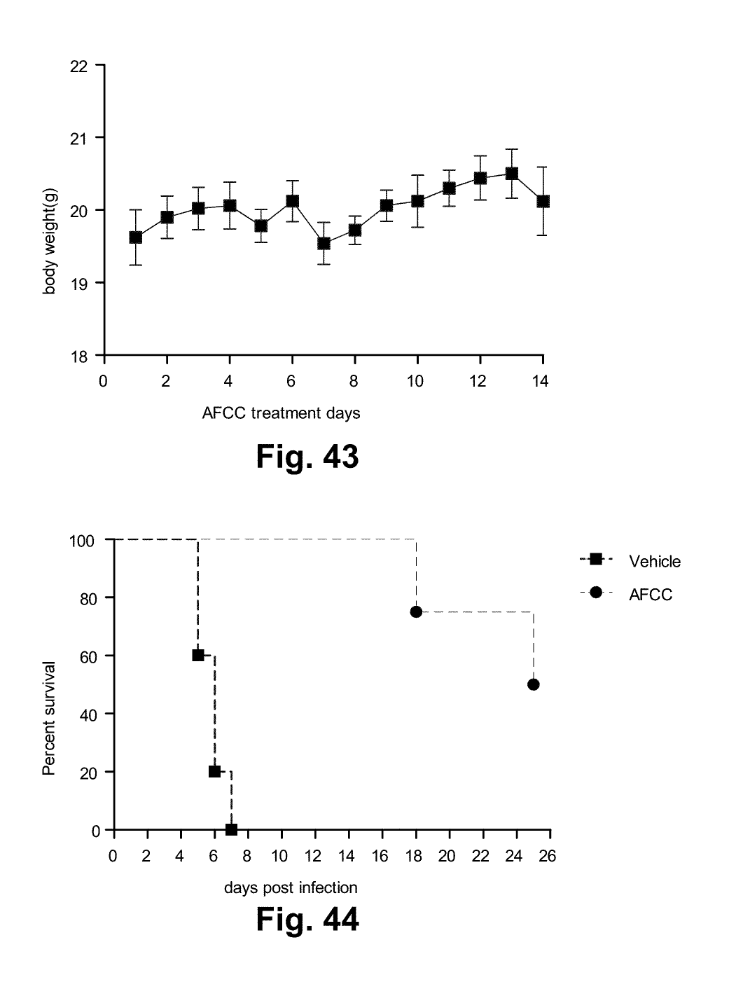

FIG. 43. Body weight changes caused with AFCC treatment in mice

TABLE 3
Effect of AFCC on mean day to death of mice infected with H1N1
A/WSN/33

| | | Mean day to death ± |
|---|---|---|
| Treatment | Survivor/total | S.D. |
| H1N1 + AFCC | 2/4 | 23.8 ± 3.0** |
| H1N1 + Vehicle | 0/5 | 5.8 ± 0.8 |

**P < 0.01 compared to the H1N1 + vehicle control

FIG. 44. Efficacy of AFCC on H1N1 WSN-caused mouse death

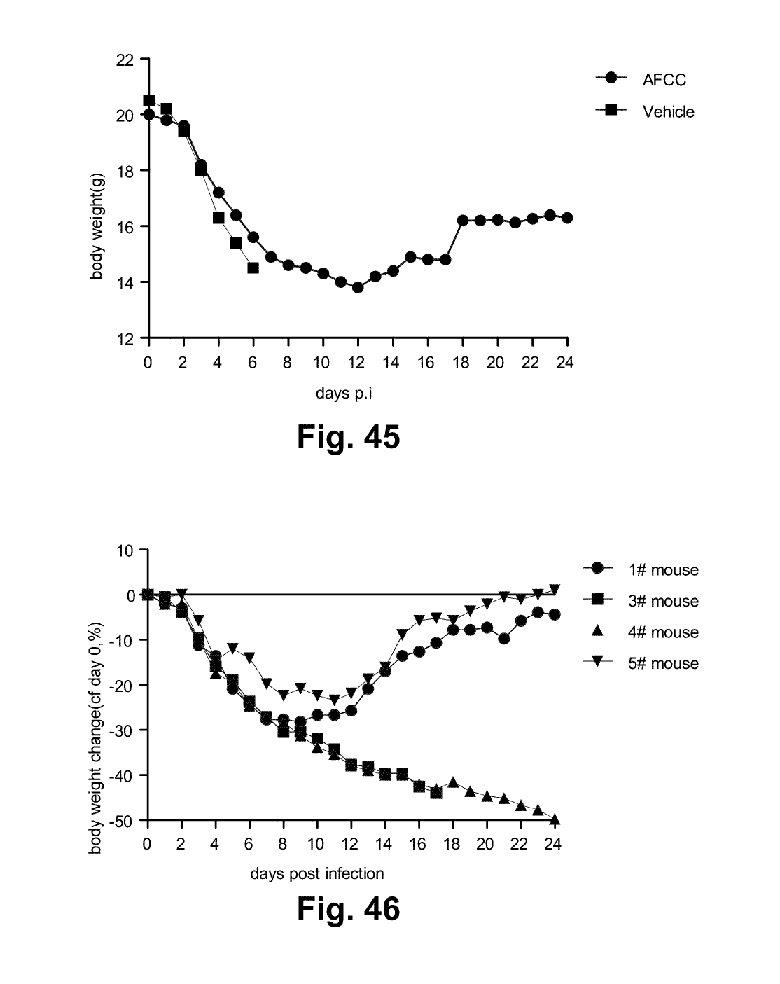

FIG. 45. Body weight changes caused by AFCC in mice infected with H1N1 (WSN) influenza

FIG. 46. Body weight change caused with AFCC treatment in mice infected with H1N1 (WSN) influenza

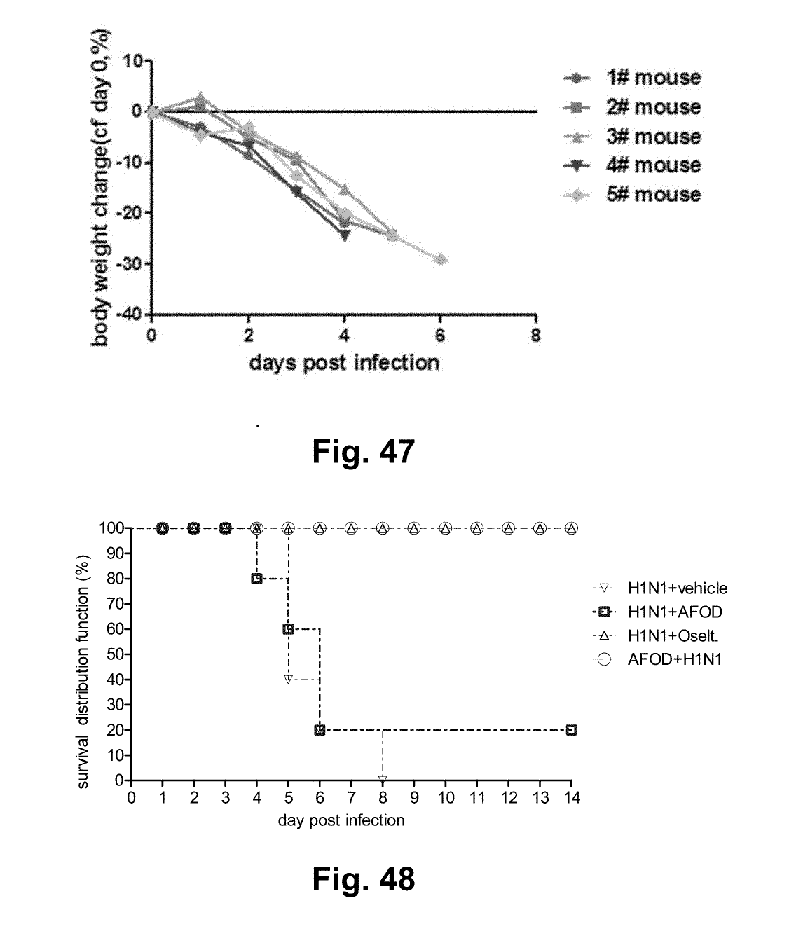

FIG. 47. Body weight change caused with Vehicle treatment in mice infected with H1N1 (WSN) influenza

Report: Antiviral Efficacy of AFOD RAAS-2 in an Influenza H1N1-Infected Mouse Model

Summary of the Report

Objective

Infection with human influenza virus (IFV) causes respiratory tract illness in human and animals including mice. Mouse model intranasally infected with IFV H1N1 is well recognized for antiviral compound screening against IFV infection. This study is designed to evaluate the compound AFOD RAAS2 from RAAS for its in vivo anti-IFV efficacy.

Study Method

This study was performed in the following steps:
1) Infect mice with IFV by intranasal inoculation.
2) Treat the mice pre or post INF infection by iv/ip dosing of the AFOD RAAS2. 3) Daily record body weight of the mice.
4) Sacrifice survived mice and inspect their major organs in the end of the study.

Result Summary

One-week preventive treatment with RAAS-2 fully protected H1N1-challenged mice from death and body weight loss although one-week therapeutic treatment with RAAS-2 led to one mouse, out of 5 mice survived in this group to the end of the experiment. In the H1N1-challenged vehicle control group all mice died and their body weights dramatically dropped by 20% to 30% within 4-7 days post-IFV H1N1 challenge. In contrast with the vehicle group, all mice treated therapeutically with oseltamivir survived although their body weights dropped and recovered to some extent. This indicated that the mouse model worked successfully in current study.

For Study Protocol: RAAS-20120428.v.2

I. Method

Animals:

Female BALB/c mice (6-8 weeks, 17-22 g) were divided into defined study groups after a visual examination and a 3 to 5-day acclimation upon arrival.

Solution Preparation:

1. Sodium Pentobarbital: Freshly dissolved in saline for injection at 7.5 mg/ml prior to using.

2. Test article: human plasma derived protein 29% AFOD RAAS2 in sterile solutions for vein injection provided by the client.

3. Vehicle: PBS

4. Oseltamivir phosphate (prodrug): aqueous solution in PBS, 0.1 mg/ml

Experimental Procedure:

IFV Infection and Test Article Administration:

1. From day −7 through day −1, 5 mice from group 4 are intravenously or intraperitoneally (iv/ip) administrated daily for 7 days.

2. On the day of Influenza administration, mice are anesthetized by intraperitoneal injection of sodium pentobarbital (80 mg/kg).

3. Anesthetized mice are inoculated with 5×10″3 pfu/mouse of Influenza H1N1 A/WSN/33 via the intranasal route in SFM medium.

4. Test article or vehicle is intravenously or intraperitoneally (iv/ip) administrated daily for 7 days. Oseltamivir (1 mg/kg) is orally given twice daily for 8 days. First dosing for oseltamivir or test article is executed 4 h pre H1N1 inoculation.

5. From day 1 through day 14 the infected mice are observed two times a day. Mortality and body weight are recorded daily.

6. On day 14, all living mice are sacrificed and dissected for the inspection of organ appearances.

II. Groups and Schedules:

TABLE 1
Action summary of the Study

| | | | IFV | AFOD, | Oseltamivir, po | | |
|---|---|---|---|---|---|---|---|
| Study | | | challenge, | iv/ip, | 10:00- | 19:50- | mouse |
| Day | Date | Weighing | 14:00-16:00 | 10:00-12:00 | 10:20 | 20:10 | sacrifice |
| Day −7 | May 22, 2012 | √ | | √ | | | |
| Day −6 | May 23, 2012 | √ | | √ | | | |
| Day −5 | May 24, 2012 | √ | | √ | | | |
| Day −4 | May 25, 2012 | √ | | √ | | | |
| Day −3 | May 26, 2012 | √ | | √ | | | |
| Day −2 | May 27, 2012 | √ | | √ | | | |
| Day −1 | May 28, 2012 | √ | | √ | | | |
| Day 0 | May 29, 2012 | √ | √ | √ | √ | √ | |
| Day 1 | May 30, 2012 | √ | | √ | √ | √ | |
| Day 2 | May 31, 2012 | √ | | √ | √ | √ | |
| Day 3 | Jun. 1, 2012 | √ | | √ | √ | √ | |
| Day 4 | Jun. 2, 2012 | √ | | √ | √ | √ | |
| Day 5 | Jun. 3, 2012 | √ | | √ | √ | √ | |
| Day 6 | Jun. 4, 2012 | √ | | | √ | √ | |
| Day 7 | Jun. 5, 2012 | √ | | | | | |
| Day 8 | Jun. 6, 2012 | √ | | | | | |
| Day 9 | Jun. 7, 2012 | √ | | | | | |
| Day 10 | Jun. 8, 2012 | √ | | | | | |
| Day 11 | Jun. 9, 2012 | √ | | | | | |
| Day 12 | Jun. 10, 2012 | √ | | | | | |
| Day 12 | Jun. 11, 2012 | √ | | | | | |
| Day 13 | Jun. 12, 2012 | √ | | | | | |
| Day 14 | Jun. 13, 2012 | √ | | | | | √ |

√ indicates that the action was taken.

TABLE 2
Experimental Design for the efficacy study

| | | | | | | | H1N1 |
|---|---|---|---|---|---|---|---|
| | | | | Vol | Treatment | 1^{st} treatment | (PFU/mo |
| Group | Mice | Compound | Dose | (ml/kg) | Schedule | time | use) |
| 1 | 5 | Vehicle^{#} | 0.2/0.4 | — | Iv/ip, QD* | 4 hrs | 5 × 10\hat{ }3 |
| | | | ml/mouse | | | pre-infection | |
| 2 | 5 | AFOD RAAS | 0.2/0.4 | — | Iv/ip, QD* | 4 hrs | 5 × 10\hat{ }3 |
| | | 2 | ml/mouse | | | pre-infection | |
| 3 | 5 | Oseltamivir | 1 mg/kg | 10 | po, BID** | 4 hrs | 5 × 10\hat{ }3 |
| | | phosphate | | | | pre-infection | |
| 4 | 5 | AFOD RAAS | 0.2/0.4 | — | Iv/ip, QD* | 7 days | 5 × 10\hat{ }3 |
| | | 2 | ml/mouse | | | pre-infection | |

Iv/ip, QD*: Iv/ip means that iv injection is carried out with the volume indicated in “dose” column on day 0, 1, 2, 4 and ip injection is carried out on day 3; QD: daily (QD) for 4 days after H1N1 inoculation;
**BID, twice daily.
Vehicle^{#}: PBS

III Adverse Events and Tolerability of Compounds:
1. On day 5 post H1N1 infection, hematuria occurred in group 2 of AFOD RAAS2 treatment. We stopped AFOD RAAS2 medication on the sixth day post H1N1 infection.
2. One mouse in the oseltamivir group died day 3 post H1N1 challenge. Its body dissection indicated that its esophagus was damaged probably due to harsh oral gavage. Therefore this mouse was ruled out from the experiment.

Result and Discussion

In the H1N1-challenged vehicle control group all 5 mice died and their body weights dramatically dropped by 20% to 30% within 4-8 days post-IFV H1N1 challenge (FIG. 1, FIG. 2, and Table 3). In contrast with the vehicle group, 4 out of 5 mice in the oseltamivir group survived to the end of experiment (FIG. 1, FIG. 2, and Table 3) although one mouse died accidentally of harsh oral gavage, which should be ruled out from the experiment as suggested early (see Part III, 2 in this report). The body weights in this group dropped by <15% days 5 to 8 post H1N1 challenge and recovered thereafter to some extent (FIG. 2). This indicated that the mouse model worked successfully in current study.

Impressively one-week preventive treatment with 0.2 ml/0.4 ml/mouse iv/ip QD of RAAS-2 totally protected H1N1-challenged mice from death and body weight loss till the end of this study (FIG. 1, FIG. 2 and Table 3). The protection of body weight loss by the preventive treatment of RAAS-2 is even better than that by oseltamivir treatment (FIG. 2). However the therapeutic treatment with 0.2 ml/0.4 ml iv/ip QD of RAAS-2 only protected one mouse out of 5 mice in the group from death and partial body weight loss of all 5 mice days 2 to 5 post H1N1 infection. Other 4 mice in this group died days 4 to 6 post H1N1 infection. In addition, some of the mice in the RAAS-2 therapeutic group (G2) had hematuria day 5 post H1N1 challenge and afterward, indicating that the dose used in this group was beyond mouse tolerance in H1N1 challenge status.

We don't understand why the RAAS-2 displayed such significant preventive efficacy on mouse death and body weight loss caused by H1N1 challenge. We have a number of suggestions to fully establish and understand this efficacy. First, we need to expand the efficacy experiment using a few more mice each group to confirm the data due to the small experiment scale (5 mice each group only) in the current study. In addition, a longer term study should be designed to fully know how long the preventive efficacy of the blood-derived product RAAS-2 could last. For example the mice should be challenged with H1N1 two weeks, three weeks, four weeks and even longer, respectively, post one week of preventive treatment of the RAAS-2. Some well designed mechanism studies should be carried out, such as in vivo H1N1 replication in infected mouse lungs in the preventive treatment and control groups, detection of immunological markers to reflect immune system activation and other biomarker assays post preventive treatment and H1N1 challenge. Finally a dose-dependent observation should be carried out for the RAAS-2 preventive treatment.

FIG. 48. Effect of AFOD RAAS2 on H1N1-caused mouse mortality

TABLE 3
Effect of AFOD RAAS2 or Oseltamivir on mean day to death (MDD) of
mice infected with H1N1 A/WSN/33

| | | | | Mean |
|---|---|---|---|---|
| | | | | day to death ± |
| Treatment | Dose | | Survivor/total | S.D. |
| H1N1 + Vehicle | 0.2/0.4 | ml/mouse | 0/5 | 4.8 ± 1.3 |
| H1N1 + AFOD | 1 | mg/kg | 1/5 | 6.2 ± 4.4 |
| RAAS2 | | | | |
| H1N1 + | 0.2/0.4 | ml/mouse | 4/4 | >14 ± 0.0*** |
| Oseltamivir | | | | |
| AFOD RAAS2 + | 0.2/0.4 | ml/mouse | 5/5 | >14 ± 0.0*** |
| H1N1 | | | | |

***P < 0.001 compared to the H1N1 + vehicle control

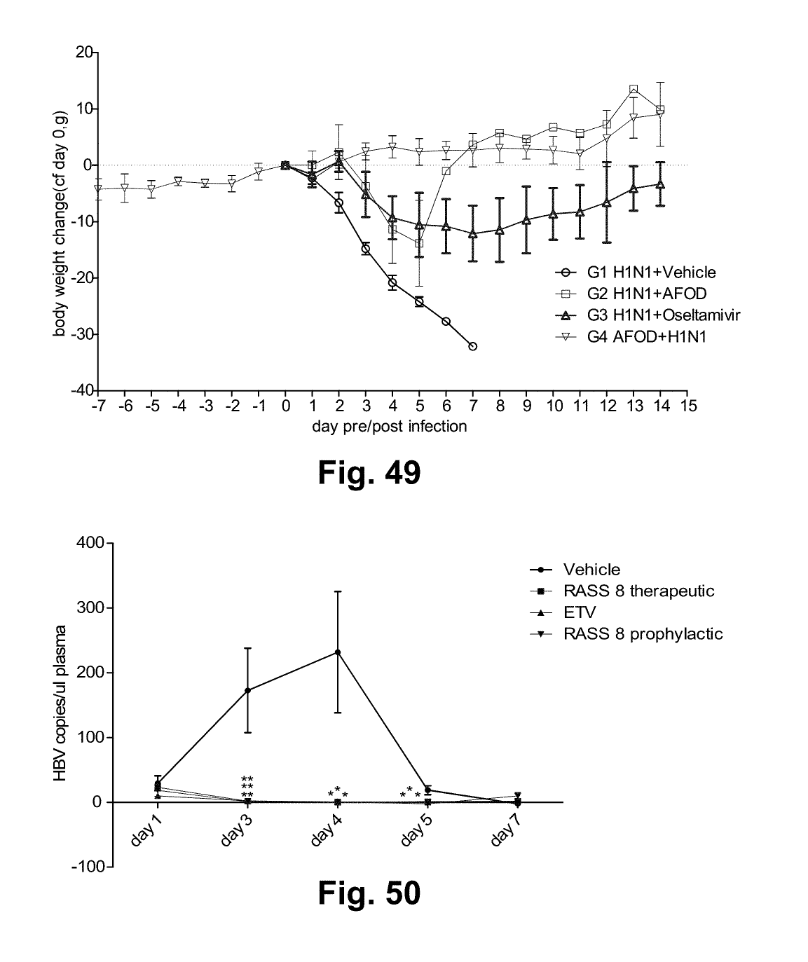

FIG. 49. The average body weight change in mice infected with H1N1 influenza

Study Report

Efficacy of RAAS-8 in the HBV Mouse Hydrodynamic Injection Model

1 Introduction

Hydrodynamic injection (HDI) is an in vivo gene delivery technology. It refers to transiently transfect the mouse liver cells with a foreign gene via tail vein injection of a large volume saline containing plasmid within a few seconds. Taking the advantage of the liver-targeting manner of hydrodynamic injection, a single hydrodynamic injection of a replication-competent HBV DNA, could result in HBV replication in mouse liver shortly. This HBV hydrodynamic injection model on immunocompetent mice is a convenient and reproducible animal model for anti-HBV compound screening in vivo, which has been successfully established in WuXi ID department.

The purpose of this study is to evaluate in vivo anti-HBV efficacy of RASS 8 using the mouse hydrodynamic injection model.

2 Materials and Reagents

2.1. Animal:

Female BALB/c mice, age 6-8 weeks, between 18˜22 g.

2.2. Test article:

Vehicle: normal saline.

Entecavir (ETV): supplied as powder by [symbol] [symbol] dissolved in normal saline prior to dosing.

AFOD-RAAS 8 (RAAS 8): provided by RAAS, 25% (blood-derived proteins) solution.

2.3. Reagent:

HBV plasmid DNA:

pcDNA3.1/HBV, prepared with Qiagen EndoFree Plasmid Giga Kit; QIAamp 96 DNA Kit, Qiagen 51162; Universal PCR Master Mix, ABI 4324020; HBV DIG DNA probe, prepared by PCR DIG Probe Synthesis Kit, Roche 11636090910; DIG Wash and Block Buffer Set, Roche 11585762001; HBsAg ELISA kit, Kehua.

3 Experimental Procedure

3.1 Hydrodynamic Injection and Compound Administration
3.1.1. From day −7 to day 0, all 5 mice in group 4 were administrated i.p./i.v. with test article daily for 8
   days according to Table 2.
3.1.2. On day 0, all groups of mice were hydrodynamicly injected via tail vein with pcDNA3.1/HBV plasmid DNA in a volume of normal saline equal to 8% of a mouse body weight. The plasmid DNA solution for injections was prepared one day before injection and then stored in 4E until injection.
3.1.3. From day 0 to day 5, mice in groups 1-3 were weighed and treated with compounds or vehicle according to the regimen in Table 2. For groups 1 and 3, the first dosing was executed 4 hours pre HDI. For groups 2, the first dosing was executed 4 hours post HDI. For group 4, the last dosing was carried out 4 hours post HDI.
3.1.4. All mice were submandibularly bled for plasma preparation according to the design in Table 1.
3.1.5. All mice were sacrificed and dissected to obtain livers (two pieces of left lobe, one piece of middle lobe and one piece of right lobe) according to the regimen in table 1. Isolated livers were snap frozen in liquid nitrogen immediately upon collected.

TABLE 1
Experimental Design for the pilot experiment

| | | | | | | | plasmid DNA injection | | | |
|---|---|---|---|---|---|---|---|---|---|---|
| | | | | Vol | Treatment | | μg/ | treatment | | |
| Group | Mice | CPD | Dose | (ml/kg) | Schedule | 1^{st} treatment time | mouse | schedule | bleeding | liver dissection |
| 1 | 5 | Vehicle# | See Tab 2 | — | See Table 2 | 4 hrs pre-injection | 20 | tail vein HDI | days | day 7 |
| 2 | 5 | RAAS 8 | See Tab 2 | — | See Table 2 | 4 hrs | | of pcDNA | 1, 3, 4, | day 7 |
| | | | | | | post-injection | | 3.1 HBV, | 5, 7 | |
| 3 | 5 | ETV | 0.1 mg/kg | 10 | PO, QD*, | 4 hrs | | | day 0, q.d. | day 5 |
| | | | | | days 0-4 | pre-injection | | | | |
| 4 | 5 | RAAS 8 | See Tab 2 | — | See Table 2 | last dosing, | | | | day 7 |
| | | | | | | 4 hrs | | | | |
| | | | | | | post-injection | | | | |

QD*: once a day;
Vehicle#: normal saline

TABLE 2
Schedule for Compound administration

| group | Day | −7 | −6 | −5 | −4 | −3 | −2 | −1 | 0 | 1 | 2 | 3 | 4 | 5 | 6 | 7 |
|---|---|---|---|---|---|---|---|---|---|---|---|---|---|---|---|---|
| 4 | am | 0.2 | 0.4 | 0.2 | 0.4 | 0.2 | 0.4 | 0.4 | HDI*, | No | No | No | No | No | No | No |
| | | ml, | ml | ml, | ml | ml | ml | ml | IV | | | | | | | |
| | | IV | IP | IV | IP | IV | IP | IP | | | | | | | | |
| | pm | No | No | No | No | No | No | No | 0.5 ml | No | No | No | No | No | No | No |
| | | | | | | | | | IP | | | | | | | |
| 2 | am | No | No | No | No | No | No | No | HDI, | 0.2 | 0.5 | 0.2 | 0.5 | No | No | No |
| | | | | | | | | | IV | ml | ml | ml | ml | | | |
| | | | | | | | | | | IV | IP | IV | IP | | | |
| | pm | No | No | No | No | No | No | No | 0.5 ml | 0.3 | No | 0.3 | No | No | No | No |
| | | | | | | | | | IP | ml | | ml | | | | |
| | | | | | | | | | | IP | | IP | | | | |
| 1 | am | No | No | No | No | No | No | No | 0.5 ml | 0.2 | 0.5 | 0.2 | 0.5 | No | No | No |
| | | | | | | | | | IP | ml | ml | ml | ml | | | |
| | | | | | | | | | | IV | IP | IV | IP | | | |
| | pm | No | No | No | No | No | No | No | HDI, | 0.3 | No | 0.3 | No | No | No | No |
| | | | | | | | | | IV | ml | | ml | | | | |
| | | | | | | | | | | IP | | IP | | | | |

HDI*: hydrodynamic injection

3.2 Sample Analysis

3.2.1 Detect HBV DNA Replication Level in Plasma
3.2.1.1 Isolate DNA from 50 μl plasma using QIAamp 96 DNA Blood Kit. DNA was eluted with 120 μl ddH₂O.
3.2.1.2. Run qPCR for HBV DNA quantification.
   a) Dilute HBV plasmid standard by 10-fold from 10⁷ copies/μl to 10 copies/μl.
   b) Prepare qPCR mix as shown below.

| PCR reagents | Volume | Volume for 100 Reactions |
|---|---|---|
| DEPC Water | 1.1 μl | 110 μl |
| Taqman Universal Master Mix | 12.5 μl | 1250 μl |
| (2X) | | |
| HBV Primer Forward (50 μM) | 0.2 μl | 20 μl |
| HBV Primer Reverse (50 μM) | 0.2 μl | 20 μl |
| HBV Probe (5 μM) | 1 μl | 100 μl |
| Total | 15 μl | 1500 μl |

c) Add 15 μl/well PCR mix to 96-well optical reaction plates.
d) Add 10 μl of the diluted plasmid standard.
e) Transfer 10 μl of the extracted DNA to the other wells. Seal the plates with optical adhesive film. Mix and centrifuge.
f) Place the plates into qPCR machine and run the program according to the table blow.

| | | |
|---|---|---|
| 50° C. | 2 min | 1 cycle |
| 95° C. | 10 min | 1 cycle |
| 95° C. | 15 s | 40 cycle |
| 60° C. | 60 s | |

To eliminate the influence of input HBV plasmid, primers and probe targeting HBV sequence which detect newly replicated HBV DNA and input HBV plasmid DNA and targeting pcDNA3.1 plasmid backbone sequence which only detect the input plasmid DNA were used to do real-time PCR, respectively.

HBV DNA quantity=DNA determined by HBV primer-DNA determined by plasmid primer.

3.2.2 Detect HBsAg Level in Plasma

Dilute the plasma 500 fold;

Detect HBsAg level in 50 μl diluted plasma by using HBsAg ELISA kit.

3.2.3 Detect HBV Intermediate DNA Level in Livers

3.2.3.1 Liver DNA Isolation
a) Homogenize the liver tissue with Qiagen Tissue Lyser in 10 mM Tris.HCl, 10 mM EDTA, pH7.5.
b) Spin samples. Transfer the supernatant to a new tube containing equal volume of 2× proteinase K digestion buffer. Incubate at 50□ for 3 hours.
c) Extract with phenol: chloroform: Isoamyl alcohol.
d) Transfer the upper phase to new tubes, add RNase A and incubate at 37° C. for 30 min.
e) Extract with phenol: chloroform: Isoamyl alcohol.
f) Transfer the upper phase to new microfuge tubes, add 0.7-1 volume of isopropanol, add GlycoBlue Coprecipitant to 50 μg/mL, incubate at −20□ for 30 min.
g) Centrifuge (12000 g, 10 min) to precipitate DNA.
h) Wash the precipitate with 70% ethanol. Dissolve it in 25 μl ddH₂O. Store DNA at −20□ until use.

3.2.3.2 qPCR for HBV DNA Quantification with Total Liver DNA.

The total liver DNA was diluted to 10 ng/μl. Use 10 μl diluted sample to run real-time PCR.

HBV DNA quantity=DNA determined by HBV primer-DNA determined by plasmid primer.

3.2.3.3 Southern Blot to Detect HBV Intermediate DNA Level in Livers.
a) Load 50 μg DNA for each sample. Run 1.2% agarose gel in 1×TAE.
b) After denaturing the gel with 0.25 M HCl at RT, neutralize the gel with neutralizing buffer.
c) Transfer the DNA form the gel to a pre-wet positively charged nylon membrane by upward capillary transfer overnight.
d) Remove the nylon membrane from the gel transfer assembly, UV cross-link the membrane (700 Microjoules/cm²), then wash it in 2×SSC for 5 min. Place the membrane at RT until dry.
e) Prehybridize membrane for 1 hour with hybridization buffer.
f) Pour off hybridization solution, and add the hybridization/pre-heated probe mixture, overnight
g) After hybridization and stringency washes, rinse membrane briefly in washing buffer.
h) Incubate the membrane in blocking solution, then in Antibody solution.
i) After wash in washing buffer, equilibrate in Detection buffer.
j) Place membrane with DNA side facing up on a development folder (or hybridization bag) and apply CDP-Star, until the membrane is evenly soaked. Immediately cover the membrane with the second sheet of the folder to spread the substrate evenly and without air bubbles over the membrane.
k) Squeeze out excess liquid and seal the edges of the development folder. Expose to X-ray film.
l) Expose to X-ray film at 15-25° C.

4 Results and Discussion

To investigate the effect of tested compounds on HBV replication in hydrodynamic model, the level of HBV DNA in plasma was analyzed by real-time PCR method (FIG. 1). Because the injected HBV plasmid DNA can also be detected by the primers targeting to HBV sequence, the primers and probe targeting the backbone sequence of pcDNA3.1 vector were designed and used for real-time PCR to eliminate the influence of residual plasmid in blood. The HBV quantity was calculated by the quantity determined by primers targeting HBV sequence subtracted by quantity determined by primers targeting the plasmid backbone sequence.

The results indicated that RASS 8 significantly inhibited the HBV replication by therapeutic or prophylactic treatment in a time-dependent manner post HDI. On day 1, RASS 8 therapeutic treatment showed ˜23% inhibition and RASS 8 prophylactic treatment showed ˜37% inhibition to HBV replication. On day 3 and day 4, the inhibition percentage to HBV replication by RASS 8 therapeutic, or prophylactic treatment was >99%, which is statistically significant. On day 5, RASS 8 therapeutic treatment caused ˜93% inhibition while its prophylactic treatment made almost 100% inhibition. The HBV level in both RAAS 8 prophylactic and therapeutic groups recovered a little on day 7 compared to the data on day 5. As a reference compound for the HBV HDI model, entecavir had significant inhibition to the HBV replication in the therapeutically-treated mice from day 3 post HDI to the end of experiment.

FIG. 50. Efficacy of therapeutic treatment or prophylactic treatment of RAAS 8 or ETV on in vivo HBV replication in HBV mouse HDI model. The total DNA was isolated from plasma by QIAamp 96 DNA Blood Kit. The HBV viral load in plasma during the course of the experiment was quantified by real-time PCR. Data is expressed as mean±SE. * P<0.05, ** P<0.01 by Student's t-test.

Secreted HBV surface proteins are also important index for HBV replication. HBsAg level in plasma was

detected by ELISA method (FIG. 2). Both RASS 8 therapeutic and prophylactic treatment had a significant inhibitory effect on HBsAg level in plasma within 5 days post HBV HDI while ETV didn't have significant inhibition to the HBsAg generation, suggesting that the in vivo effect of RAAS 8 on the in vivo HBV replication may be through a different mechanism from the entecavir.

FIG. 51. Effect of prophylactic treatment or therapeutic treatment of RAAS 8 or ETV on the HBsAg in mouse blood. The HBsAg level in plasma during the course of the experiment was determined by HBsAg ELISA kit. Data is expressed as mean±SE. * P<0.05, ** P<0.01 by Student's t-test.

Hepatitis B virus is a member of the hepadnavirus family, which replicates in livers and depends on liver specific factors. Thus, the existence of intermediate DNA in livers is a direct evidence for HBV replication in livers. To quantify the intermediate HBV DNA in livers, the total DNA was isolated from liver and HBV DNA level was determined by real-time PCR (FIG. 3). ETV, as a positive control, significantly decreased the HBV intermediate DNA in liver on day 5 Similar to ETV, RASS 8 prophylactic treatment had a significant inhibition on the replication of HBV intermediate DNA in livers on day 7. In comparison to the prophylactic treatment of RAAS 8, its therapeutic treatment caused significant but to less extent inhibition to the liver HBV replication by real time PCR (FIG. 3).

The HBV quantity determined by real-time PCR is total copy number of rcDNA, dsDNA and ssDNA. To separate and visualize rcDNA, dsDNA and ssDNA, southern blot was performed (FIG. 4). The major form of HBV replication intermediate DNA was ssDNA, which was consistent with report in literatures. Due to the limitation of DIG DNA probe sensitivity, we were not able to detect rcDNA or dsDNA. ssDNA decreased dramatically after RASS 8 prophylactic treatment or ETV treatment (FIG. 4), which confirms the result by real-time PCR (FIG. 3).

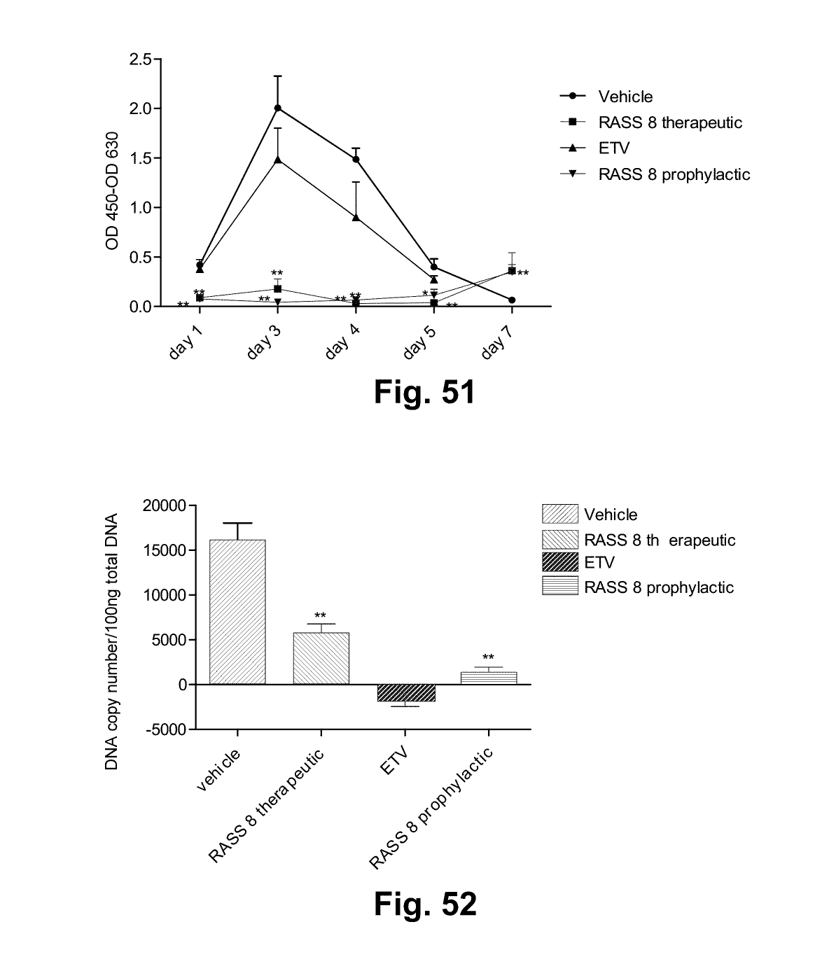

FIG. 52. Effect of prophylactic treatment or therapeutic treatment of RAAS 8 or ETV on the intermediate HBV replication in the mouse livers by qPCR. Mice in ETV group were sacrificed on day 5 and mice in the other three groups were sacrificed on day 7 post HDI. Liver DNA was isolated and subjected to real-time PCR to quantify the level of HBV replication intermediate DNA. Data is expressed as mean±SE. **P<0.01 by Student's t-test.

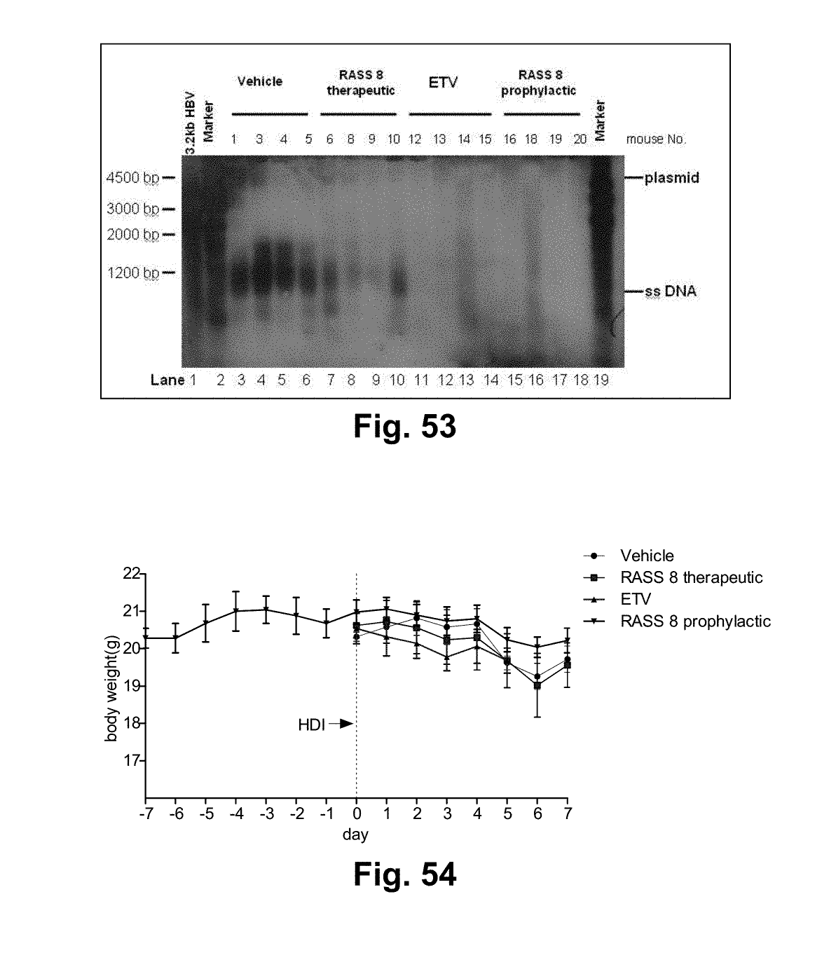

FIG. 53. Southern blot determination of intermediate HBV DNA in mouse livers. 50 μg total DNA each was subjected to southern blot. Lane 1 is 3.2 kb fragment of HBV plasmid (100 pg). Lane 2 and lane 19 are DNA makers. Lanes 3 to 18 are samples.

FIG. 54. The body weights of mice treated with vehicle or indicated compounds during the course of experiment

In summary, the RAAS 8 significantly inhibited HBV DNA replication by prophylactic or therapeutic treatment in the current study with the mouse HDI model. Impressively the prophylactic treatment with RAAS 8 displayed stronger inhibition to the HBV replication than its therapeutic treatment although we need more experiment to understand this phenomenon. In this study only 5 mice were used in each group. Thus the result may need to be confirmed by using more animals. In addition a well-designed mechanism study may be required to clarify how the RAAS 8 protein functions against HBV infection.

Efficacy of a Human Plasma Derived Protein AFOD RAAS 105 in Inhibition of the HBV Replication in the Mouse Hydrodynamic Injection Model

1.0 Introduction

Hydrodynamic injection (HDI) is an in vivo gene delivery technology. It refers to transiently transfect the mouse liver cells with a foreign gene via tail vein injection of a large volume saline containing plasmid within a few seconds. Taking the advantage of the liver-targeting manner of HDI, a single HDI of a replication-competent HBV DNA, could result in HBV replication in mouse liver. This HBV HDI model on immunocompetent mice is a convenient and reproducible animal model for anti-HBV compound screening in vivo. The purpose of this study is to evaluate in vivo anti-HBV efficacy of RAAS 105 using the mouse HDI model.

Materials and Reagents

Animal:

Female BALB/c mice (6-8 weeks old), between 18˜22 g, specific pathogen free, are purchased from SLAC (Shanghai Laboratory Animal Center of Chinese Academy of Sciences) and housed in an animal care facility in individually ventilated cages. Guidelines are followed for the care and use of animals as indicated by WuXi IACUC (Institutional Animal Care and Use Committee). The study was approved by WuXi IACUC (IACUC protocol 20120104-mouse). Mice are allowed to acclimate to the new environment for 3-5 days and are grouped according to the experimental set up.

Test Article:

Vehicle: normal saline.

Entecavir (ETV): supplied as powder by Rongda Pharm & Chem Co. Ltd., dissolved in normal saline prior to dosing.

AFOD RAAS 105: 25% protein concentrations (human plasma derived protein), provided by RAAS.

Reagents:

HBV Plasmid DNA:

pcDNA3.1/HBV, prepared with Qiagen EndoFree Plasmid Giga Kit, supplied as 850 ng/μl solution, diluted in normal saline.

QIAamp 96 DNA Kit, Qiagen 51162; Universal PCR Master Mix, ABI 4324020; HBV DIG DNA probe, prepared by PCR DIG Probe Synthesis Kit, Roche 11636090910; DIG Wash and Block Buffer Set, Roche 11585762001; HBsAg ELISA kit, Kehua.

Experimental Procedure

HDI and Compound Administration

From day −7 to day 0, mice in groups 6-7 are administrated IP/IV by turn with test article daily for 8 days according to Table 3.

On day 0, mice from group 1 to group 7 are hydrodynamically injected through tail vein with pcDNA3.1/HBV plasmid DNA in a volume of normal saline equal to 8% of a mouse body weight within 5 seconds. The plasmid DNA solution for injections is prepared before injection.

From day 0 to day 4, mice in groups 3-5 are administrated IP/IV by turn with test article daily for 5 days according to Table 2. From day 0 to day 4, mice in groups 1 and 2 are administrated PO with vehicle or ETV daily for 5 days according to Table 1. For groups 1-5, the first dosing is executed 4 hours post hydrodynamic injection. For groups 6-7, the last dosing is executed 4 hours pre hydrodynamic injection.

Mice from group 1 to group 7 are submandibularly bled for plasma preparation according to the design in Table 1. The blood samples will be collected in a tube containing heparin sodium and centrifuged at 7000×g and 4° C. for 10 min to obtain plasma samples.

On day 5, all mice in groups 1-7 are sacrificed and dissected to obtain livers (two pieces of left lobe, one piece of middle lobe and one piece of right lobe) according to the regimen in table 1. Livers are snap frozen in liquid nitrogen immediately upon collected.

TABLE 1
Grouping and Treatment

| | | Test article | | | | | Plasmid DNA | | | |
|---|---|---|---|---|---|---|---|---|---|---|
| | | | | | | 1^{st} | injection | | | |
| | | | | | | or last | Dose | | | |
| | Mice/ | Group | | In vol | | dosing | μg/ | Treatment | | sacrifice |
| Group | group | ID | Dose | (ml/kg) | schedule | time | mouse | schedule | Bleeding | time |
| 1 | 10 | vehicle | — | 10 | PO, QD, | 1^{st}, 4 hrs | ~20 | HDI | 0.2 | Day 5 |
| | | | | | days 0-4 | post-HDI | | pcDNA | ml/mouse, | |
| 2 | | ETV | 0.1 | 10 | PO, QD, | 1^{st}, 4 hrs | | 3.1/HB | days | |
| | | | mg/kg | | days 0-4 | post-HDI | | V, | 1, 3, 4, 5 | |
| 3 | | Therapeutic | 0.15 | — | See | 1^{st}, 4 hrs | | day 0, | | |
| | | RAAS | ml/ | | Table 2 | post-HDI | | QD | | |
| | | 105 | mouse | | | | | | | |
| 4 | | | 0.25 | — | See | 1^{st}, 4 hrs | | | | |
| | | | ml/ | | Table 2 | post-HDI | | | | |
| | | | mouse | | | | | | | |
| 5 | | | 0.35 | — | See | 1^{st}, 4 hrs | | | | |
| | | | ml/ | | Table 2 | post-HDI | | | | |
| | | | mouse | | | | | | | |
| 6 | | Prophylactic | 0.35 | — | See | last, 4 hrs | | | | |
| | | vehicle | ml/ | | Table 3 | pre-HDI | | | | |
| | | | mouse | | | | | | | |
| 7 | | Prophylactic | 0.35 | — | See | last, 4 hrs | | | | |
| | | RAAS | ml/ | | Table 3 | pre-HDI | | | | |
| | | 105 | mouse | | | | | | | |

TABLE 2
Dosing schedule for therapeutic groups

| group | compound | day | 0 | 1 | 2 | 3 | 4 |
|---|---|---|---|---|---|---|---|
| 3 | Therapeutic | am | HDI IV | 0.15 ml | 0.15 ml | 0.15 ml | 0.15 ml |
| | RAAS 105 | | | IP | IV | IP | IV |
| | | pm | 0.15 ml | — | — | — | — |
| | | | IP | | | | |
| 4 | | am | HDI IV | 0.25 ml | 0.25 ml | 0.25 ml | 0.25 ml |
| | | | | IP | IV | IP | IV |
| | | pm | 0.25 ml | — | — | — | — |
| | | | IP | | | | |
| 5 | | am | HDI IV | 0.35 ml | 0.35 ml | 0.35ml | 0.35 ml |
| | | | | IP | IV | IP | IV |
| | | pm | 0.35 ml | — | — | — | — |
| | | | IP | | | | |

TABLE 3
Dosing schedule for prophylactic groups

| Group | Compound | Day | −7 | −6 | −5 | −4 | −3 | −2 | −1 | 0 |
|---|---|---|---|---|---|---|---|---|---|---|
| 6 | Prophylactic | am | 0.35 | 0.35 ml | 0.35 | 0.35 ml | 0.35 | 0.35 ml | 0.35 | 0.35 |
| | vehicle | | ml IP | IV | ml IP | IV | ml IP | IV | ml IP | ml IP |
| | | pm | — | — | — | — | — | — | — | HDI |
| | | | | | | | | | | IV |
| 7 | Prophylactic | am | 0.35 | 0.35 ml | 0.35 | 0.35 ml | 0.35 | 0.35 ml | 0.35 | 0.35 |
| | RAAS | | ml IP | IV | ml IP | IV | ml IP | IV | ml IP | ml IP |
| | 105 | pm | — | — | — | — | — | — | — | HDI |
| | | | | | | | | | | IV |

Sample Analysis

Detect HBV DNA Replication Level in Plasma

Isolate DNA from 50 μl plasma using QIAamp 96 DNA Blood Kit. DNA was eluted with 120 μl ddH₂O.

Run qPCR for HBV DNA quantification.
a. Dilute HBV plasmid standard by 10-fold from 10′ copies/μl to 10 copies/μl.
b. Prepare qPCR mix as shown below.

| | | Volume for |
|---|---|---|
| PCR reagents | Volume | 100 Reactions |
| DEPC Water | 1.1 μl | 110 μl |
| Taqman Universal Master | 12.5 μl | 1250 μl |
| Mix (2X) | | |
| HBV Primer Forward (50 μM) | 0.2 μl | 20 μl |
| HBV Primer Reverse (50 μM) | 0.2 μl | 20 μl |
| HBV Probe (5 μM) | 1 μl | 100 μl |
| Total | 15 μl | 1500 μl |

c. Add 15 μl/well PCR mix to 96-well optical reaction plates.
d. Add 10 μl of the diluted plasmid standard.
e. Transfer 10 μl of the extracted DNA to the other wells. Seal the plates with optical adhesive film. Mix and centrifuge.
f. Place the plates into qPCR machine and run the program according to the table blow.

| | | |
|---|---|---|
| 50° C. | 2 min | 1 cycle |
| 95° C. | 10 min | 1 cycle |
| 95° C. | 15 s | 40 cycle |
| 60° C. | 60 s | |

To eliminate the influence of input HBV plasmid, primers and probe targeting HBV sequence which detect newly replicated HBV DNA and input HBV plasmid DNA and targeting pcDNA3.1 plasmid backbone sequence which only detect the input plasmid DNA were used to do real-time PCR, respectively.

HBV DNA quantity=DNA determined by HBV primer-DNA determined by plasmid primer.

Detect HBsAg Level in Plasma
Dilute the plasma 200 fold;
Detect HBsAg level in 50 μl diluted plasma by using HBsAg ELISA kit.

Detect HBV Intermediate DNA Level in Livers

Liver DNA Isolation
a. Homogenize the liver tissue with Qiagen Tissue Lyser in 10 mM Tris.HCl, 10 mM EDTA, pH7.5.
b. Spin samples. Transfer the supernatant to a new tube containing equal volume of 2× proteinase K digestion buffer. Incubate at 50□ for 3 hours.
c. Extract with phenol: chloroform: Isoamyl alcohol.
d. Transfer the upper phase to new tubes, add RNase A and incubate at 37° C. for 30 min.
e. Extract with phenol: chloroform: Isoamyl alcohol.
f. Transfer the upper phase to new microfuge tubes, add 0.7-1 volume of isopropanol, add GlycoBlue Coprecipitant to 50 μg/mL, incubate at −20□ for 30 min.
g. Centrifuge (12000 g, 10 min) to precipitate DNA.
h. Wash the precipitate with 70% ethanol. Dissolve it in 25 μl ddH₂O. Store DNA at −20□ until use.

qPCR for HBV DNA quantification with total liver DNA.

The total liver DNA was diluted to 10 ng/μl. Use 10 μl diluted sample to run real-time PCR.

HBV DNA quantity=DNA determined by HBV primer-DNA determined by plasmid primer.

Results and Discussion

To investigate the effect of the test compound on HBV replication in the mouse hydrodynamic injection model, the level of HBV DNA in plasma was analyzed by real-time PCR method (FIG. 1, FIG. 2). Because the injected HBV plasmid DNA can also be detected by the primers targeting to HBV sequence, the primers and probe targeting the backbone sequence of pcDNA3.1 vector were designed and used for real-time PCR to eliminate the influence of residual plasmid in blood. Thus the in vivo replicated HBV quantity was determined by subtracting DNA quantity amplified by primers targeting the plasmid backbone sequence from the DNA quantity amplified by primers targeting HBV sequence.

The results in FIG. 1 indicated that on day 3 and day 4 post HDI, the therapeutic treatment with 0.15, 0.25 or 0.35 ml RAAS 105/mouse inhibited the HBV DNA replication in plasma. The inhibition on day 4 was statistically significant compared to the vehicle group although the data on day 3 was not statistically significant due to the variation of HBV DNA replication. On day 5, after stopped dosing with the RAAS 105 the HBV DNA replication inhibition in plasma rebounded a little to a small extent.

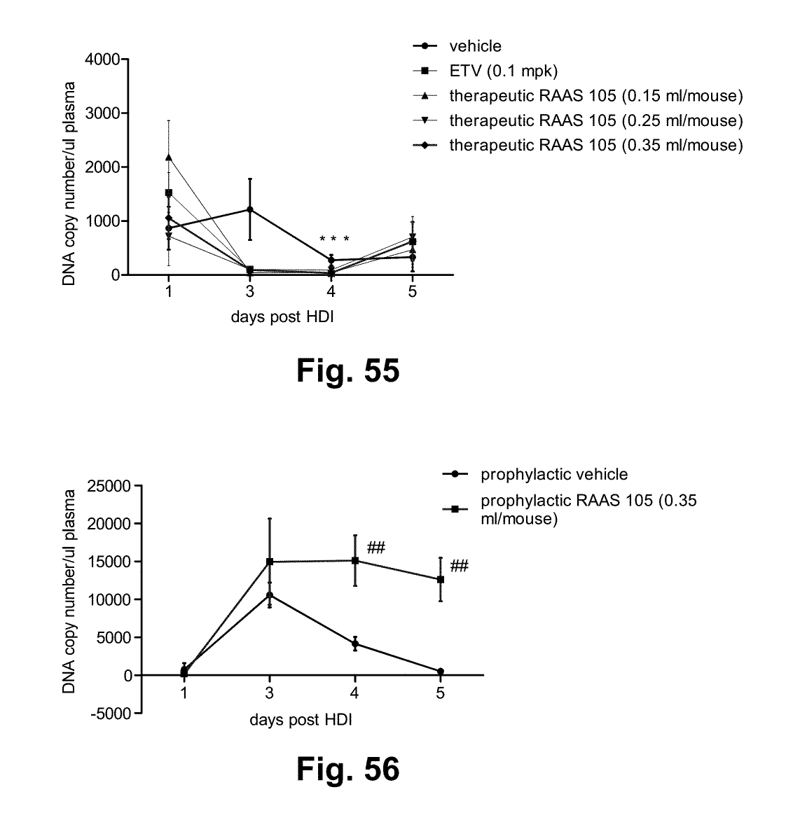

FIG. 55 Efficacy of therapeutic treatment of RAAS 105 on in vivo HBV replication in plasma in mouse HDI model. The total DNA was isolated from mouse plasma obtained at time points as indicated in the figure by QIAamp 96 DNA Blood Kit. The HBV viral load in plasma during the course of the experiment was quantified by real-time PCR. Data is expressed as mean±SE. * P<0.05 by Student's t-test.

The results in FIG. 2 indicated that the prophylactic treatment with high dose (0.35 mL) of RAAS 105 didn't inhibit the HBV replication in plasma. In the other way around, the prophylactic treatment with 0.35 ml RAAS 105/mouse for a week pre HDI resulted in a significant enhancement of HBV level in mouse plasma on days 4 and 5 post HDI. Currently the mechanism of this effect remains to be figured out.

FIG. 56 Efficacy of prophylactic treatment of RAAS 105 on in vivo HBV replication in plasma in mouse HDI model. The total DNA was isolated from mouse plasma obtained at time points as indicated in the figure by QIAamp 96 DNA Blood Kit. The HBV viral load in plasma during the course of the experiment was quantified by real-time PCR. Data is expressed as mean±SE. ^{##}P<0.01 by Student's t-test.

Secreted HBV surface proteins are also important index for HBV replication. HBsAg level in plasma was

detected by ELISA method (FIG. 3, FIG. 4).

The therapeutic treatment with 0.15, 0.25 or 0.35 ml RAAS 105/mouse had a very significant inhibitory effect on HBsAg level in plasma on days 1, 3, and 4. In contrast, oral 0.1 mg/kg ETV displayed similar pattern for the HBsAg generation to the vehicle group and ETV even caused significant increase on day 3, perhaps due to the data variation, suggesting that the in vivo effect of RAAS 105 on the in vivo HBV replication may be through a different mechanism from the entecavir.

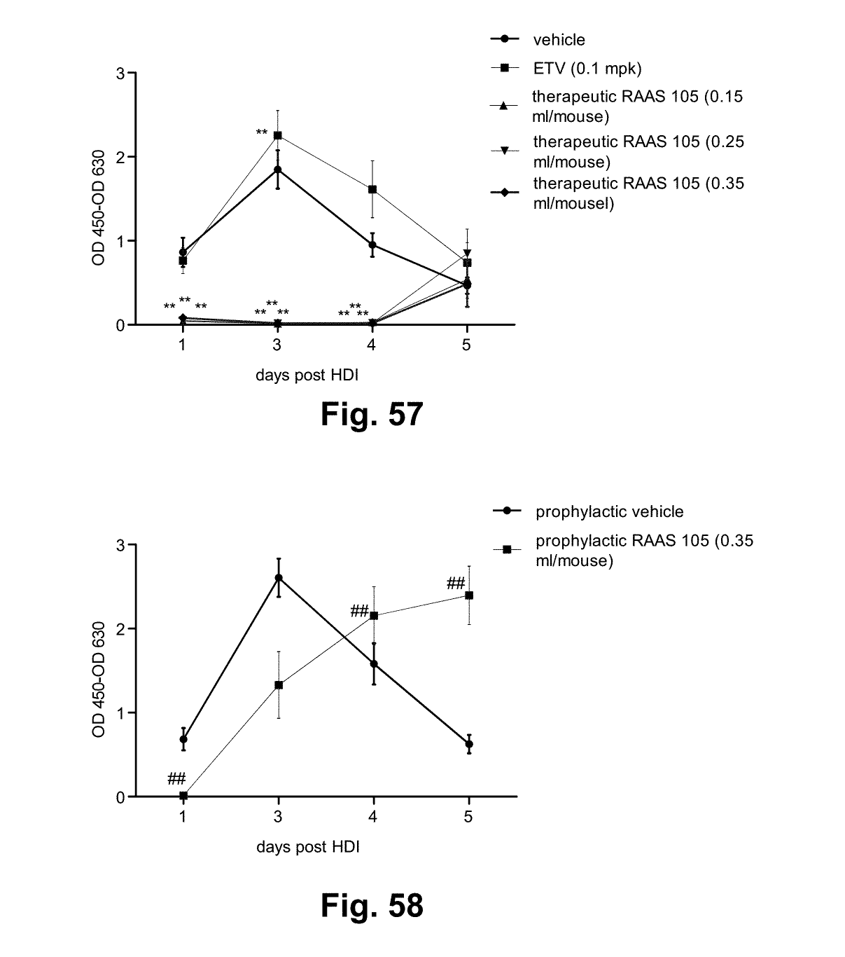

FIG. 57. Effect of therapeutic treatment of RAAS 105 on the HBsAg in mouse plasma. The HBsAg level in plasma during the course of the experiment was determined by HBsAg ELISA kit. Data is expressed as mean±SE. ** P<0.01 by Student's t-test.

The prophylactic treatment with 0.35 ml RAAS 105/mouse caused decrease of HBsAg level in plasma on day 1 and day 3 although the inhibitory effect on day 3 was not statistically significant, perhaps due to the data variation (FIG. 4). On days 4 and 5, however, the prophylactic treatment with high dose of RAAS 105 increased the HBsAg level in plasma compared with the vehicle (FIG. 4), which is somewhat consistent with the in vivo effect of RAAS 105 on the HBV load in mouse plasma (FIG. 2).

FIG. 58. Effect of prophylactic treatment of RAAS 105 on the HBsAg in mouse plasma. The HBsAg level in plasma during the course of the experiment was determined by HBsAg ELISA kit. Data is expressed as mean±SE. ^{##}P<0.01 by Student's t-test.

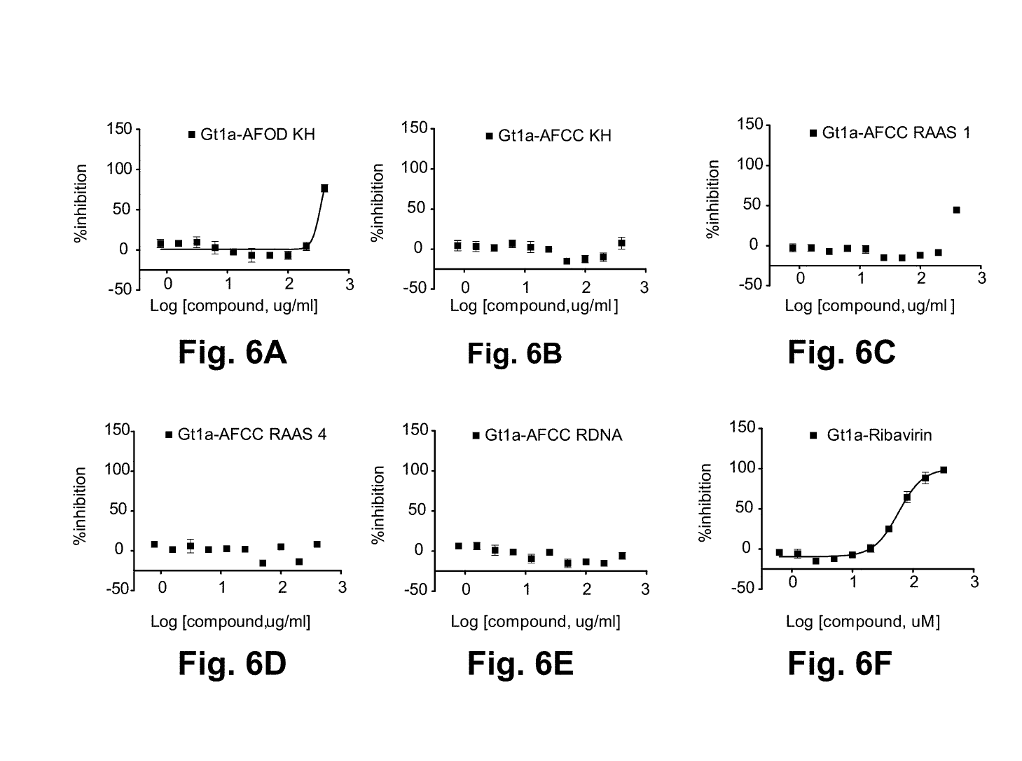

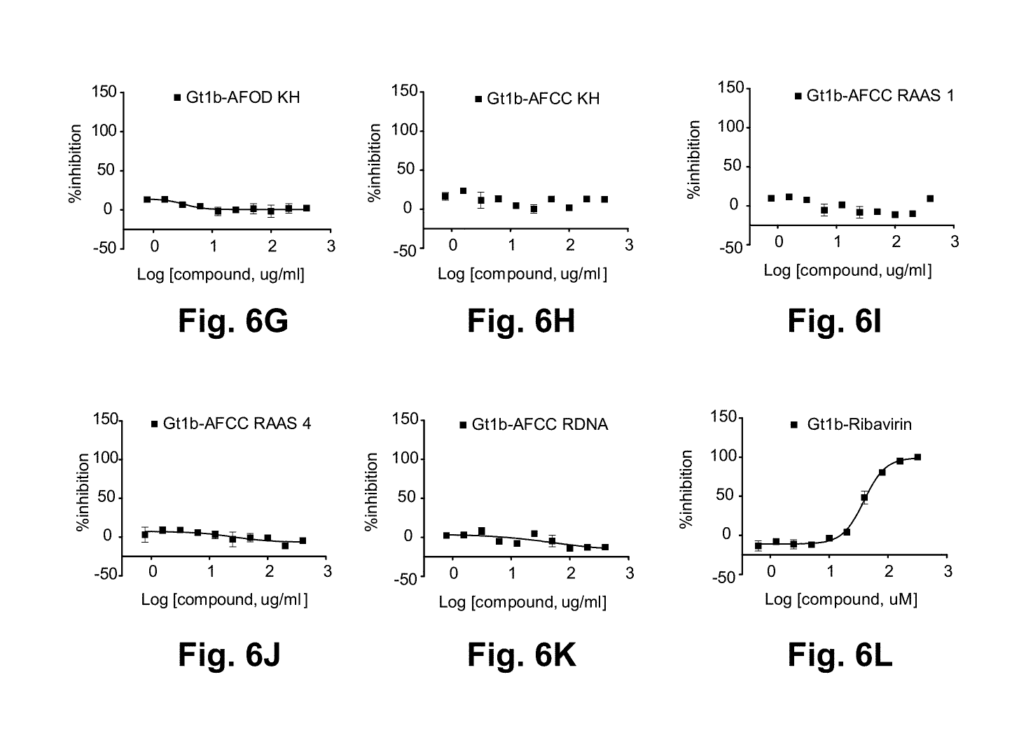

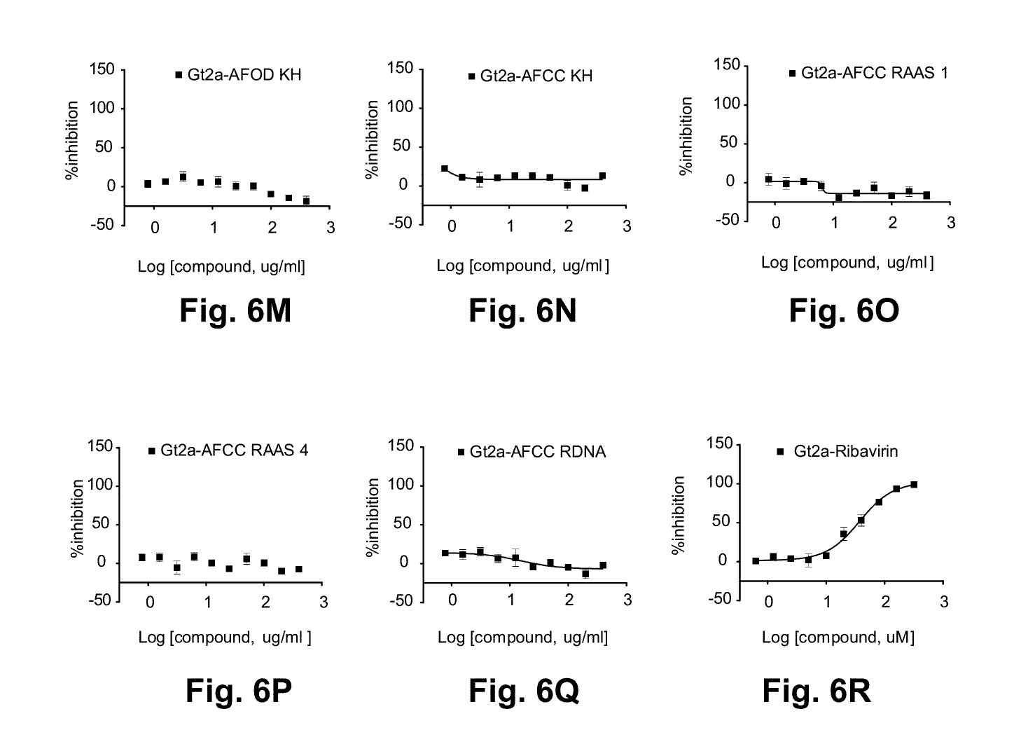

Hepatitis B virus is a member of the hepadnavirus family, which replicates in livers and depends on liver specific factors. Thus, the existence of intermediate DNA in livers is a direct evidence for HBV replication in livers. To quantify the intermediate HBV DNA in livers, the total DNA was isolated from liver and HBV DNA level was determined by real-time PCR (FIG. 5, FIG. 6).

As a reference compound for the HBV HDI model, entecavir, after 4-day oral treatment, significantly inhibited HBV replication in livers compared to the vehicle-treated group. However, RAAS 105 neither therapeutic nor prophylactic treatment showed statistically significant effect on HBV replication in livers (FIG. 5, FIG. 6), which is consistent with the in vivo effect of RAAS 105 on the HBV load in mouse plasma on day 5 (FIG. 1, FIG. 2).

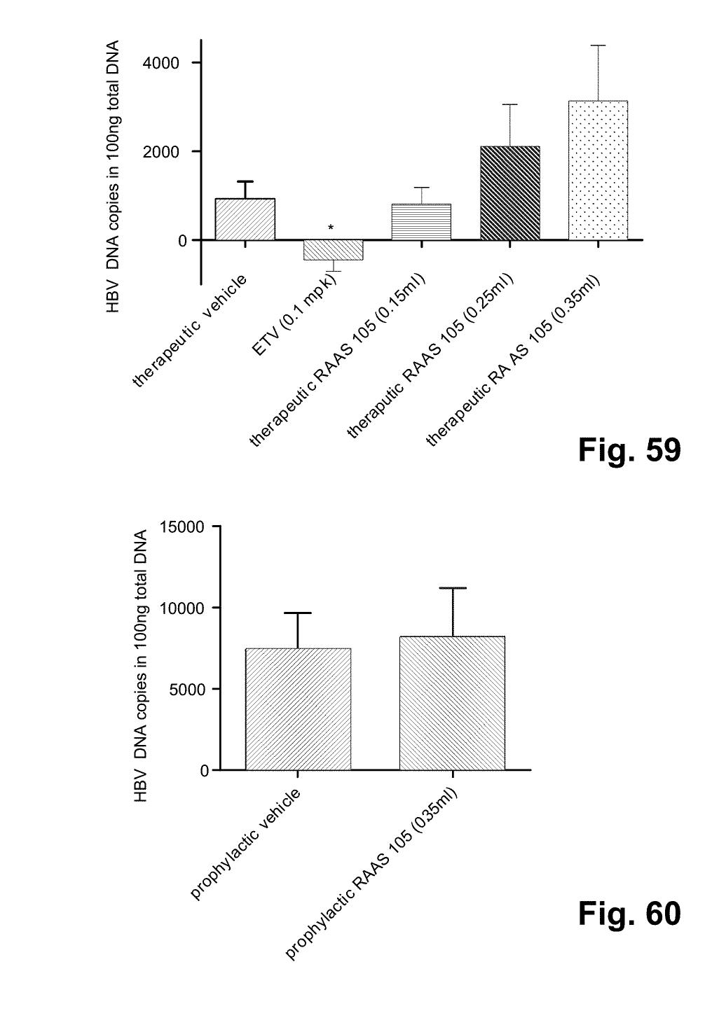

FIG. 59. Effect of therapeutic treatment of RAAS 105 on the intermediate HBV replication in the mouse livers by qPCR. Liver DNA was isolated and subjected to real-time PCR to quantify the level of HBV replication intermediate DNA. Data is expressed as mean±SE. * P<0.05 by Student's t-test.

FIG. 60. Effect of prophylactic treatment of RAAS 105 on the intermediate HBV replication in the mouse livers by qPCR. Mouse livers were dissected in the end of experiment (day 5), liver DNA was isolated and subjected to real-time PCR to quantify the level of HBV intermediate DNA. Data is expressed as mean±SE.

Mouse body weight in all groups was monitored daily over the study period. During the time course, mice in endpoint assessment control groups and experimental groups did not differ significantly in terms of changes in body weight from baseline values (FIG. 7), indicating that no toxicity presented in mice treated with vehicle or test compound doses as indicated in the FIG.

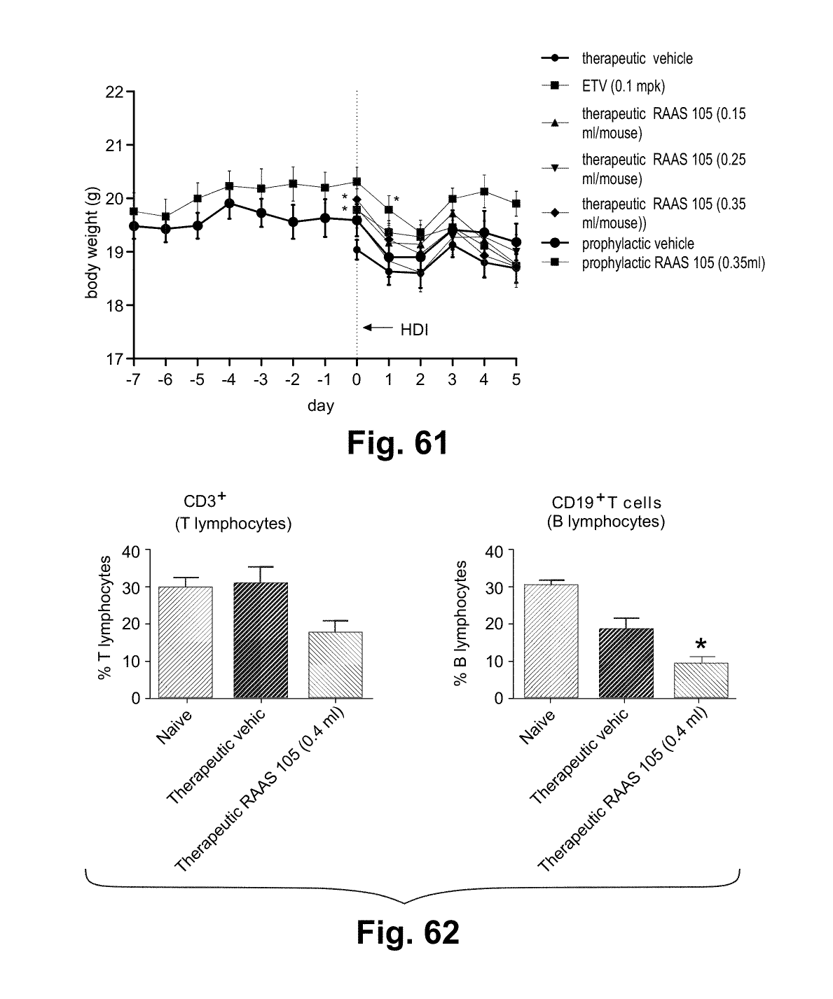

FIG. 61. The body weights of mice in all groups treated with vehicle or indicated compounds during the course of experiment (n=10). * P<0.05 by Student's t-test.

Conclusion

The therapeutic treatment with RAAS 105 displayed significant inhibition on HBV DNA replication in plasma and a very significant inhibitory effect on HBsAg level in plasma. The prophylactic treatment with 0.35 ml/mouse RAAS 105 didn't have any inhibition on HBV DNA replication in plasma on days 1 and 3 post HDI, and instead increased HBV DNA level in mouse plasma on days 4 and 5 post HDI. In addition, the prophylactic treatment showed some inhibition on HBsAg level in plasma in early time points, such as day 1 post HDI.

Neither RAAS 105 therapeutic treatment nor high dose of prophylactic treatment showed any inhibition on HBV DNA replication in livers although therapeutic treatment with oral 0.1 mg/kg ETV significantly reduced the HBV DNA replication in livers on day 5 post HDI.

After the AFOD RAAS 8 and AFOD RAAS 105 have successfully inhibited the replication of Hepatitis B in mice immediately from day 1 to day 5 and the most impressive result is that AFOD RAAS 8 as well as AFOD RAAS 105 have completely eliminated the presence of Hepatitis B surface antigen in these mice. While the positive control drug ETV can only stop the replication of Hepatitis B virus. However ETV cannot kill the virus like AFOD RAAS 8 and AFOD RAAS 105. In order to know the population of cells to help these infected mice to recover a further study on the immune cell test was conducted on these three group of mice. Amazingly we discovered a new found cell like non B and non T cell.

Final Report

Characterization of Lymphoid Tissues and Peripheral Blood in HBV Infected BALB/c Mice Treated with RAAS 105

1 Executive Summary

This study was to investigate the effects of RAAS 105 on multiple cell lineages in lymphoid tissues and peripheral blood in HBV infected BALB/c mice. HBV infection and RAAS 105 treatment were performed by ID unit at Wuxi. At the termination, blood samples and lymphoid tissues were provided to us for analysis of various cell lineages by FACS.

Two independent experiments were performed. One experiment was to test therapeutic effects of RAAS 105 and the other experiment was to test prophylactic effects of RAAS 105.

Compared with the vehicle group, the differences observed in the animals treated with RAAS 105 therapeutically include: 1) percentages of T cells and B cells in peripheral blood, spleen and lymph nodes were decreased significantly; 2) CD62L was greatly downregulated on both CD4⁺ and CD8⁺ T cells in the spleen and lymph nodes; 3) granulocytes and monocytes/macrophages in peripheral blood and lymph nodes increased significantly; 4) the percentages of regulatory T cells (CD4⁺CD25⁺Foxp3⁺) in the spleen and lymph nodes were increased significantly.

However, prophylactic treatment with RAAS 105 led to somewhat different results. In the group treated with RAAS 105, T- and B-lymphocytes were also decreased. The percentages of monocytes and macrophages were increased albeit to a less degree.

These results suggested that administration of RAAS 105 had significant effects on the frequencies of immune cell lineages. However, it is not clear whether the effects were contributed by the high concentration of proteins in RAAS 105.

2 List of Abbreviations

| | |
|---|---|
| FACS | Flow Cytometry |
| mDC | Myeloid dendritic cell |
| pDC | Plasmacytoid dendritic cell |

3 Materials and Methods

3.1 Materials

3.1.1 Reagents

FITC, Rat Anti-Mouse CD4, BD, Cat: 557307

PerCP-Cy5.5, Rat Anti-Mouse CD4, BD, Cat: 550954

FITC, Rat Anti-MouseCD3 molecular complex, BD, Cat: 561798

PerCP-Cy5.5, Rat Anti-Mouse CD3, BD, Cat: 560527

PerCP-Cy5.5, Rat Anti-Mouse CD8a, BD, Cat: 551162

PE, Rat Anti-MouseCD8a, BD, Cat: 553032

PE, Rat Anti-Mouse B220/CD45R, BD, Cat: 553089

APC, Rat Anti-Mouse CD11b, BD, Cat: 553312

APC, Ar Ham Anti-Mouse CD11c, BD, Cat: 550261

PE, Rat Anti-Mouse CD62L, BD, Cat: 553151

APC, Rat Anti-Mouse CD44, BD, Cat: 559250

PE, Rat Anti-Mouse Gr-1(Ly-6G and Ly-6C), BD, Cat: 553128

Alexa Fluor® 647, Rat Anti-Mouse Foxp3, BD, Cat: 560401

PerCP-Cy5.5, Rat Anti-Mouse CD19, BD, Cat: 551001

PE, Rat Anti-Mouse CD25, BD, Cat: 553075

ACK Lysing buffer, Invitrogen, Cat: A10492-01

RPMI 1640 medium, Invitrogen Gibco, Cat: 22400105

Dulbecco's Phosphate Buffered Saline, Thermo. Cat: SH30028.01B.

Fetal bovine serum, Invitrogen Gibco, Cat: 10099141

3.1.2 Materials

Cell strainer (70 μm), BD, Cat: 352350

BD Falcon tubes (12×75 mm, 5 ml), BD, Cat: 352054

3.1.3 Equipments

Vi-CELL Cell Viability Analyzer, Beckman Coulter, Cat: 731050

FACS Caliburflow cytometer, BD, Cat: 342975

3.2 Methods

Peripheral blood was collected through cardiac puncture. After removing red blood cells with lysis buffer followed by two rounds of washing using 1×PBS, mononuclear cells (monocytes, macrophages, dendritic cells, and lymphocytes) and granulocytes were obtained. Spleen and lymph nodes cell suspension were obtained after filtering through 70 μm cell strainer. Cell viability and number were analyzed by Vi-CELL Cell Viability Analyzer followed by cell surface staining Cells were centrifuged and resuspended in staining buffer (0.08% NaN₃/PBS+1% FBS) containing appropriate fluorescent-conjugated antibodies. After 30 min incubation at 4° C. in the dark, cells were washed twice with 0.08% NaN₃/PBS (200 μl per sample), and resuspended with 400 μl 0.08% NaN₃/PBS in BD Falcon tubes (12×75 mm, 5 ml) followed by FACS analysis.

4 Data Analysis

FACS data were analyzed by flowjo software.

5 Study Summary

5.1 Study Initiation Date and Completion Date

To investigate the therapeutic and prophylactic effect of RAAS 105 on the immune system in mice infected with HBV, the study had divided into two parts. The individual part was performed on Sep. 11 and 19, 2012, respectively.

5.2 Study Purpose

The purpose of this study was to investigate the effect of RAAS 105 on cellular composition in lymphoid tissues and peripheral blood of HBV infected mice treated with RAAS 105.

5.3 Study Results

5.3.1 Effect of Therapeutic Treatment with RAAS 105

1) Mice Information

Total 10 female BALB/c mice including 2 naïve mice at the same age were transferred from Infectious Disease (ID) Group of WuxiApptec. The group and the regimen information were shown by Table 1.

TABLE 1
The experimental group and dosing regimen of the 1^{st} part of the study

| | | | | 1^{st} or last | |
|---|---|---|---|---|---|
| Groups | N | Group ID | Dose | dosing | Analysis |
| 1 | 4 | Therapeutic | — | 1^{st}, 4 hrs | Day 5 |
| | | vehicle | | post-HDI | |
| 3 | 4 | Therapeutic | 0.4 ml/mouse | 1^{st}, 4 hrs | Day 5 |
| | | RAAS 105 | | post-HDI | |
| 11 | 2 | Naive | — | — | — |

2) Cell Populations in Peripheral Blood

After removing red blood cells, T cell lineages, B cells, DCs, granulocytes, and monocytes/macrophages in peripheral blood were analyzed by FACS analysis.

Total T cells and B cells were characterized by CD3 and CD19, respectively. HBV infection did not change the percents of CD3⁺ T cells compared with naïve mice. Therapeutic treatment of RAAS 105 reduced the percents of both CD3⁺ T cells and CD19⁺B cells significantly (FIG. 1). The representative FACS profiles from each group were illustrated in FIG. 2.

FIG. 62. Percents of T and B lymphocytes in peripheral blood. Total lymphocytes were gated. After therapeutic treated by RAAS 105, percents of T/B cells significantly decreased in peripheral blood. (by t test)

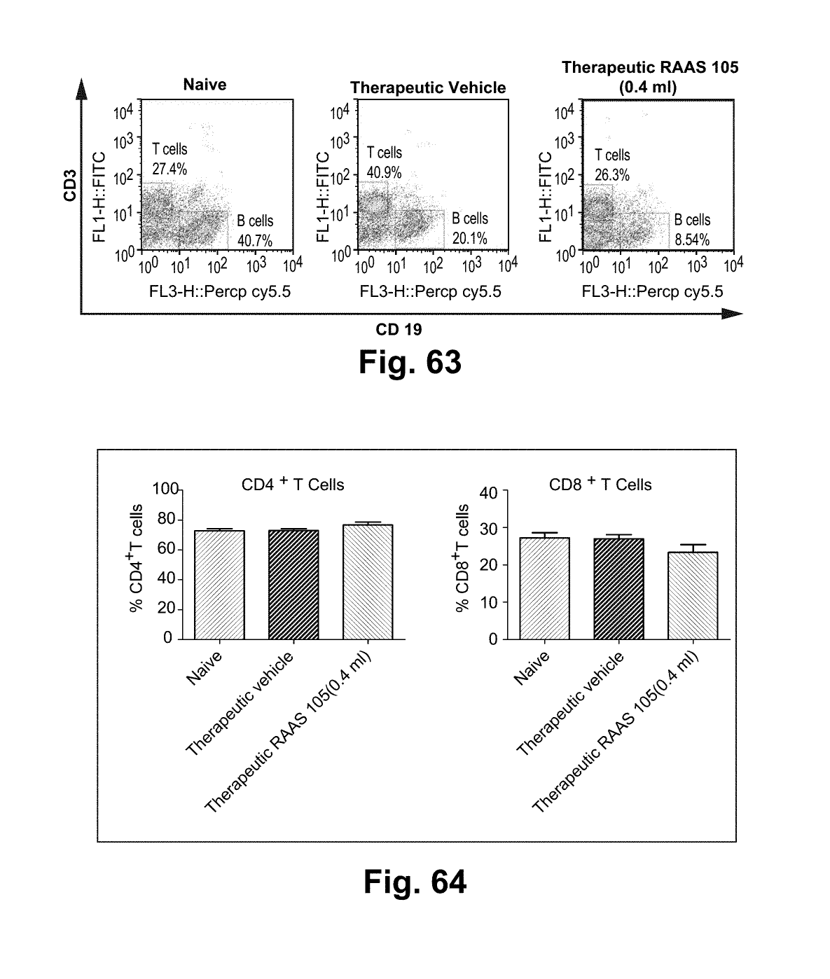

FIG. 63. Percent of T cells and B cells in peripheral blood. Total lymphocytes were gated.

Further analysis of the percents of CD4⁺ and CD8⁺ (non-CD4⁺) T cell lineages were performed gating on total CD3⁺ T cells. The results showed there were no differences in the percents of CD4⁺ and CD8⁺T cells among all the groups (FIG. 3). The representative FACS profiles from each group were illustrated in FIG. 4.

FIG. 64. Percents of CD4 and CD8 T cells in peripheral blood. Total CD3 T cells were gated and further analyzed for CD4/CD8 percentages.

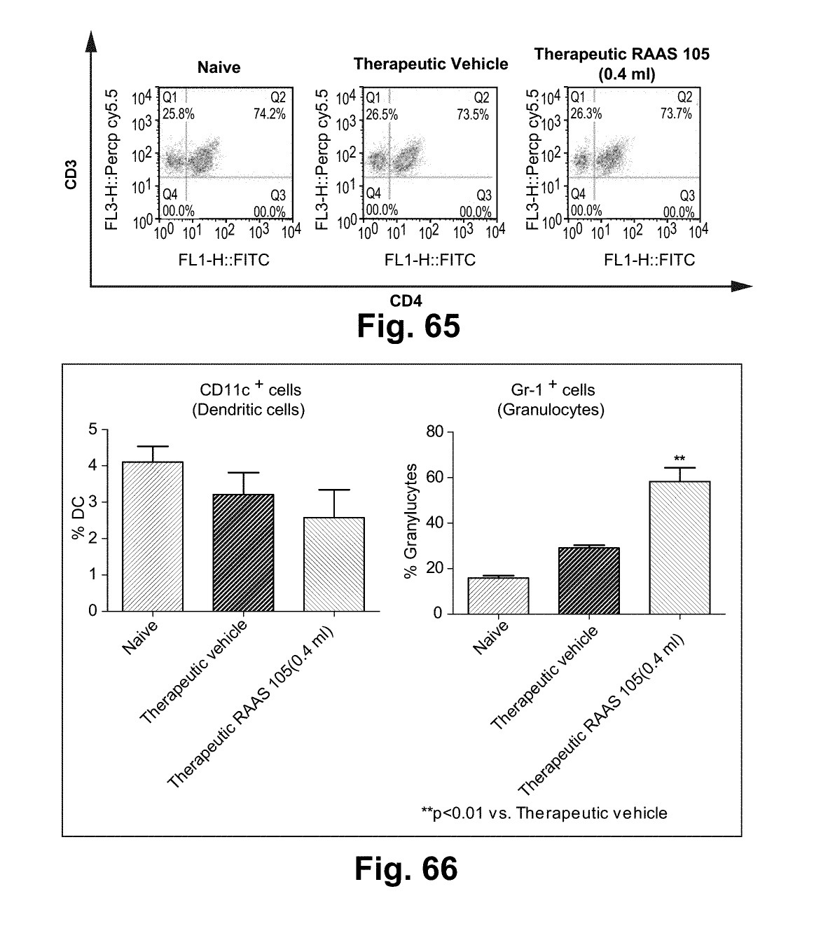

FIG. 65. Percents of CD4 and CD8 T cells in peripheral blood. Total CD3 T cells were gated.

Percents of total CD11c⁺ dendritic cells (DC) and Gr-1⁺ granulocytes in peripheral blood were investigated. HBV infection reduced the percents of CD11c⁺DCs, a phenomenon which also be observed in human patients, whereas the percents of Gr-1⁺ granulocytes were not affected. Therapeutic treatment of RAAS 105 did not show any effect on CD11c⁺DCs, but increased the percents of Gr-1⁺ granulocytes significantly (FIG. 5). The representative FACS profiles from each group were illustrated in FIG. 6.

FIG. 66. Percents of Dendritic cells and Granulocytes in peripheral blood. Total live cells were gated. After therapeutic treatment, percents of granulocytes increased in peripheral blood (by T test)

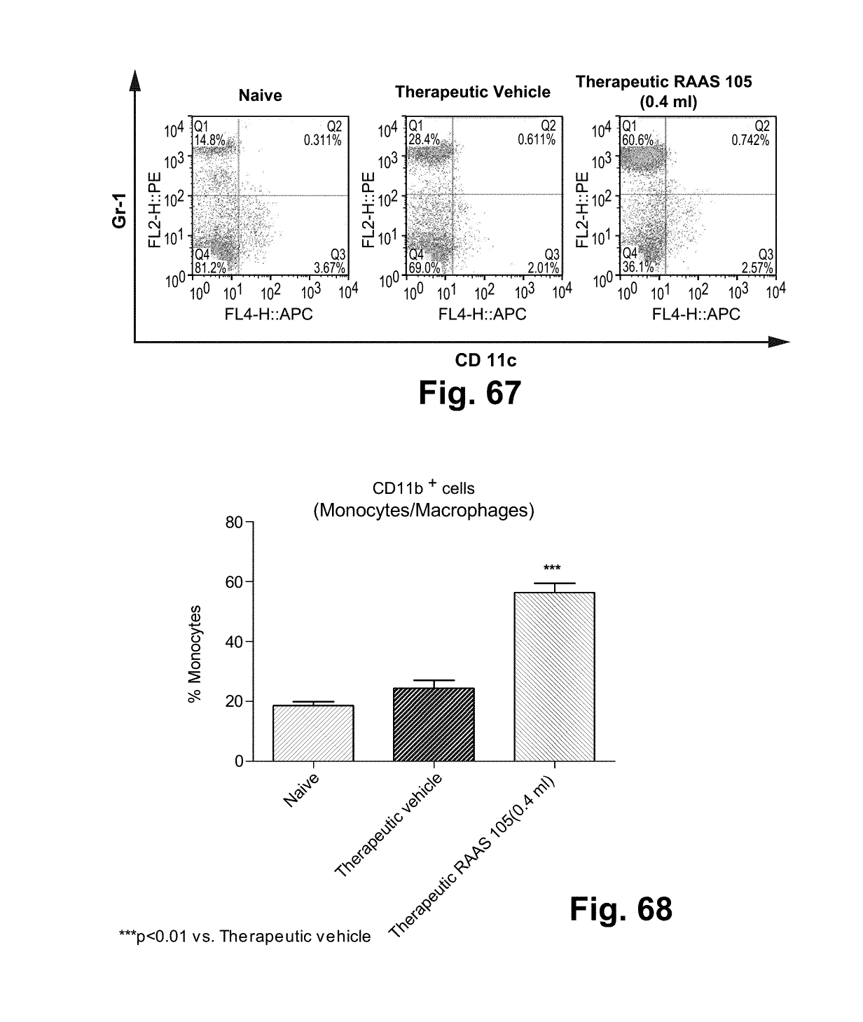

FIG. 67. Percents of Granulocytes/Dendritic cells in peripheral blood. Total live cells were gated.

Percents of Monocytes were examined using surface marker CD11b. It increased significantly as same as Gr1+ granulocytes compared with the vehicle group (FIG. 7). The representative FACS profiles from each group were illustrated in FIG. 8.

FIG. 68. Percents of Monocytes in peripheral blood. Total live cells were gated. After treatment, percents of monocytes in peripheral blood significantly increased (t test)

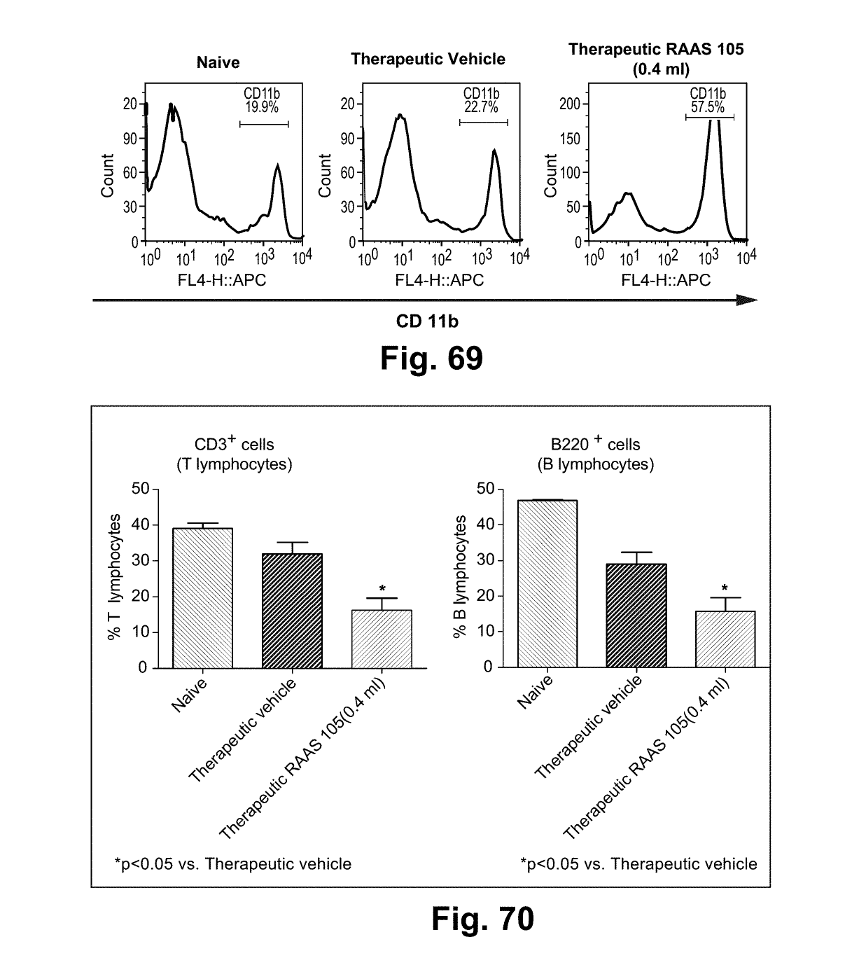

FIG. 69. Percents of monocytes in peripheral blood. Total live cells were gated.

3) Cell Populations in Spleen

Cell lineages in spleen including T cell lineages (CD4⁺/CD8⁺ T cells, naïve T cells, memory T cells and regulatory T cells), B cells, mDCs, pDCs, granulocytes and macrophages were characterized by cell surface and intracellular markers.

Percents of total T cells and B cells in spleen were investigated. Therapeutic treatment of RAAS 105 reduced the percents of both CD3⁺ T cells and CD19⁺B cells significantly (FIG. 9). The representative FACS profiles from each group were illustrated in FIG. 10.

FIG. 70. Percents of T and B lymphocytes in spleen. Total lymphocytes were gated. After therapeutic treatment by RAAS 105, percents of T cells and B cells significantly decreased in spleen.

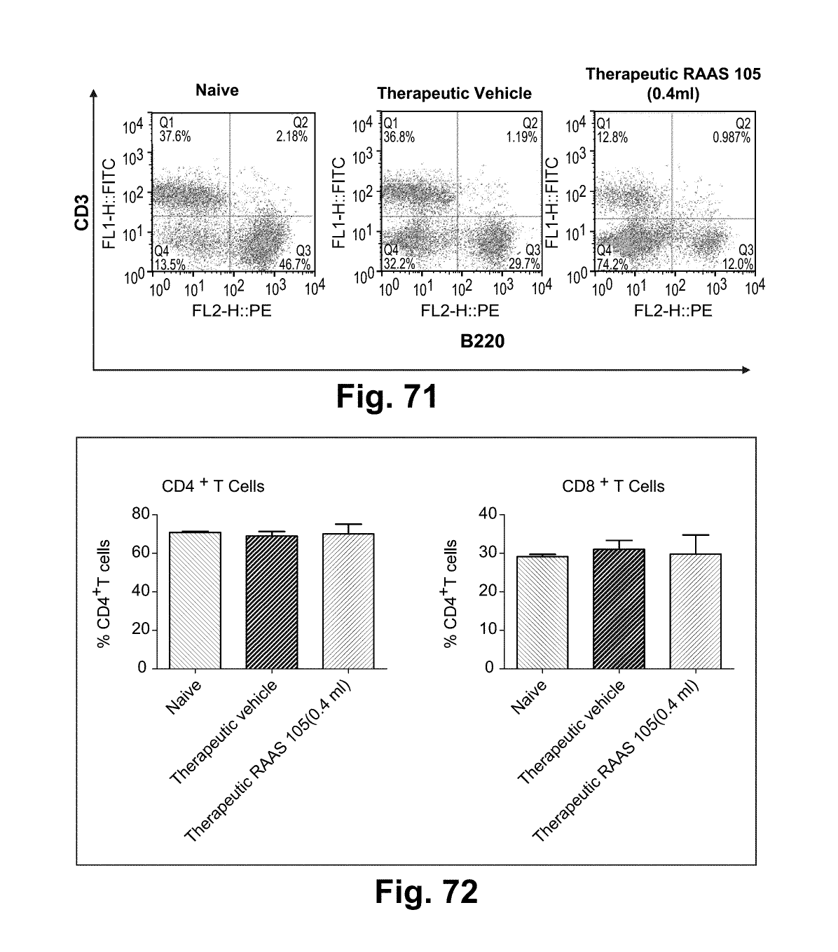

FIG. 71. Percents of T cells and B cells in spleen. Total lymphocytes were gated.

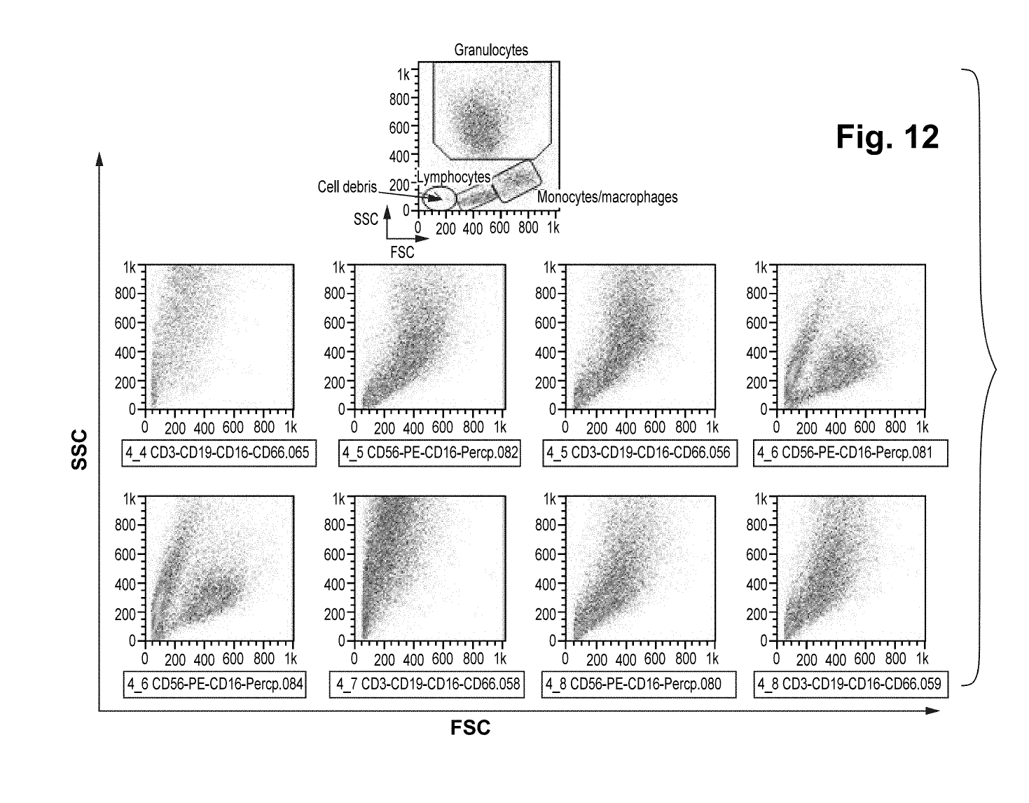

Further analysis of the percents of CD4⁺ (non-CD8⁺) and CD8⁺T cell lineages were performed gating on total CD3⁺ T cells. There were no differences in the percents of CD4⁺ and CD8⁺ T cells among all the groups (FIG. 11). The representative FACS profiles from each group were illustrated in FIG. 12.

FIG. 72. Percents of CD4 and CD8 T cells in spleen. Total CD3 T cells were gated and further analyzed for CD4/CD8 percentages.

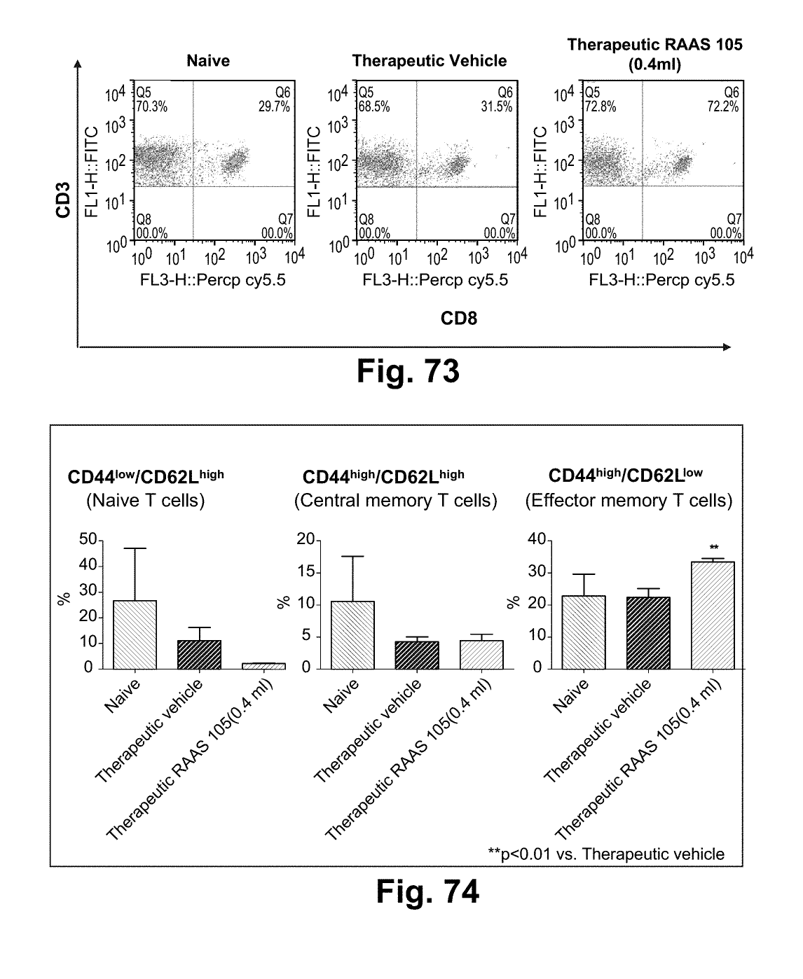

FIG. 73. Percents of CD4 and CD8 T cells in spleen. Total CD3 T cells were gated.

Three T cell lineages, naïve T cells (CD44^{low}CD62L^{high}), central memory T cells (T_{CM}s, CD44^{high}CD62L^{high}) and Effector memory T cells (T_{EM}s, CD44^{high}CD62L^{low}), were characterized by surface markers CD44 and CD62L. Percents of these T cell lineages in CD4⁺ or CD8⁺ T cells were analyzed respectively. Both in CD4⁺ and CD8⁺ T cells, percents of naïve T cells and T_{CM}s decreased and T_{EM}s increased after the therapeutic treatment of RAAS 105, suggesting the compound may have effect to promote the transformation of T cells from naïve T cells to memory T cells in spleen (FIGS. 13 and 15). The representative FACS profiles from each group were illustrated in FIGS. 14 and 16.

FIG. 74. T cell subsets percentages in spleen. Total CD4 T cells were gated and T cell subsets were determined.

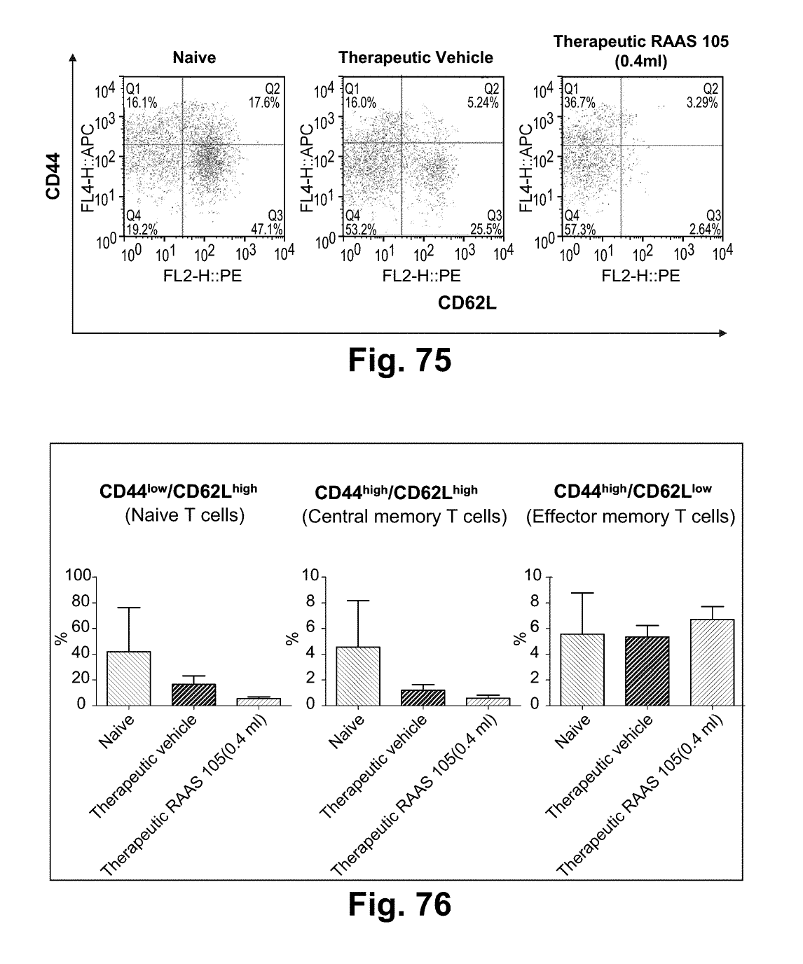

FIG. 75. CD4 T cell subsets percentages in spleen. Total CD4 T cells were gated and T cell subsets were determined.

FIG. 76. T cell subsets percentages in spleen. Total CD8 T cells were gated and T cell subsets were determined.

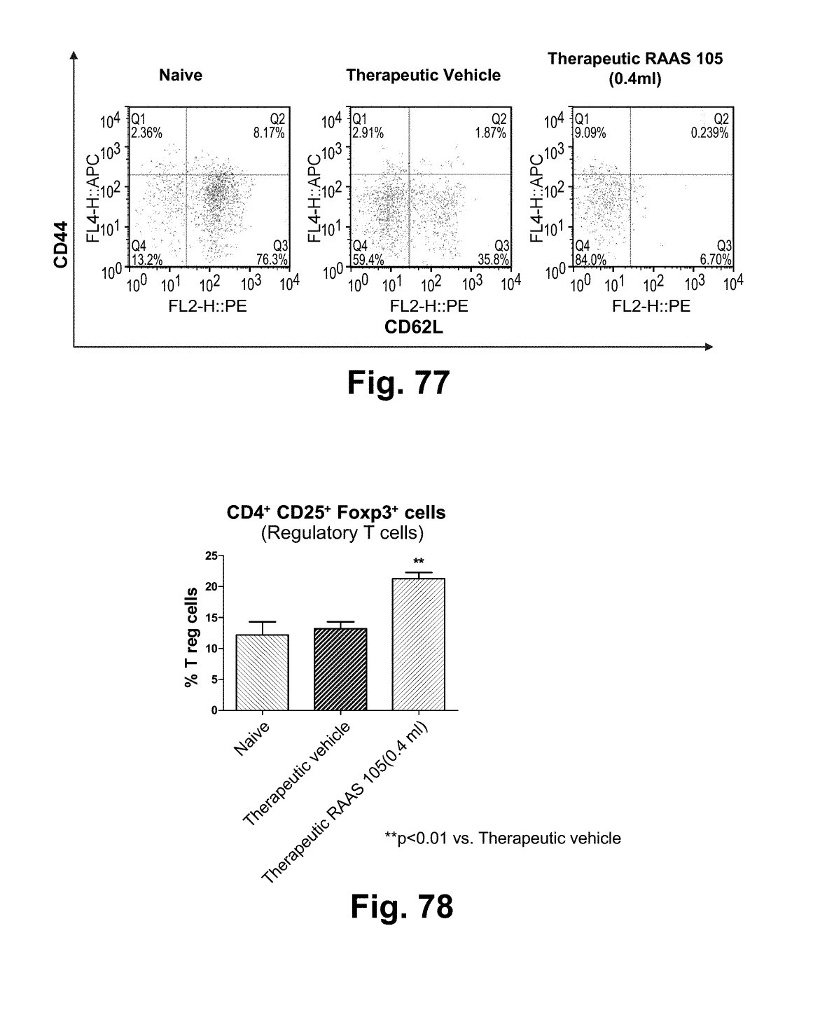

FIG. 77. CD8 T cell subsets percentages in spleen. Total CD8 T cells were gated and T cell subsets were determined.

Regulatory T cells (Tregs) were analyzed by cell surface staining of anti-CD4 and anti-CD25 antibodies followed by intracellular staining of anti-Foxp3 antibody. Percents of Tregs in spleen increased compared with the vehicle group (FIG. 17). The representative FACS profiles from each group were illustrated in FIG. 18.

FIG. 78. Percents of Foxp3 regulatory T cells in spleen. Foxp3 regulatory T cells were analyzed by intracellular staining After treatment, the percentage of T regulate cells is increased.

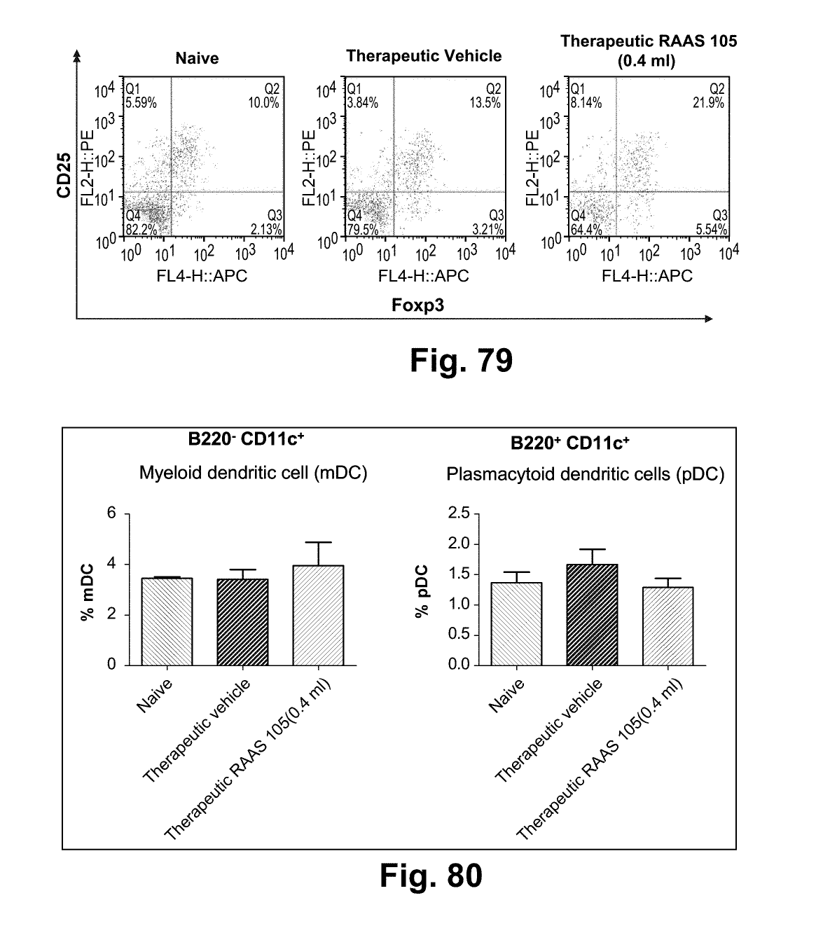

FIG. 79. Percents of regulatory T cells in spleen. Total CD4 T cells were gated.

Dendritic cells, including myeloid dendritic cells (mDC, B220⁻CD11c⁺) and plasmacytoid dendritic cells (pDC, B220⁺CD11c⁺) in spleen were analyzed. No significant differences of mDCs and pDCs were observed among all groups (FIG. 19). The representative FACS profiles from each group were illustrated in FIG. 20.

FIG. 80. Percents of pDcs and mDcs in spleen. Total live cells were gated. There were no significant differences after compound treatment. (by t test)

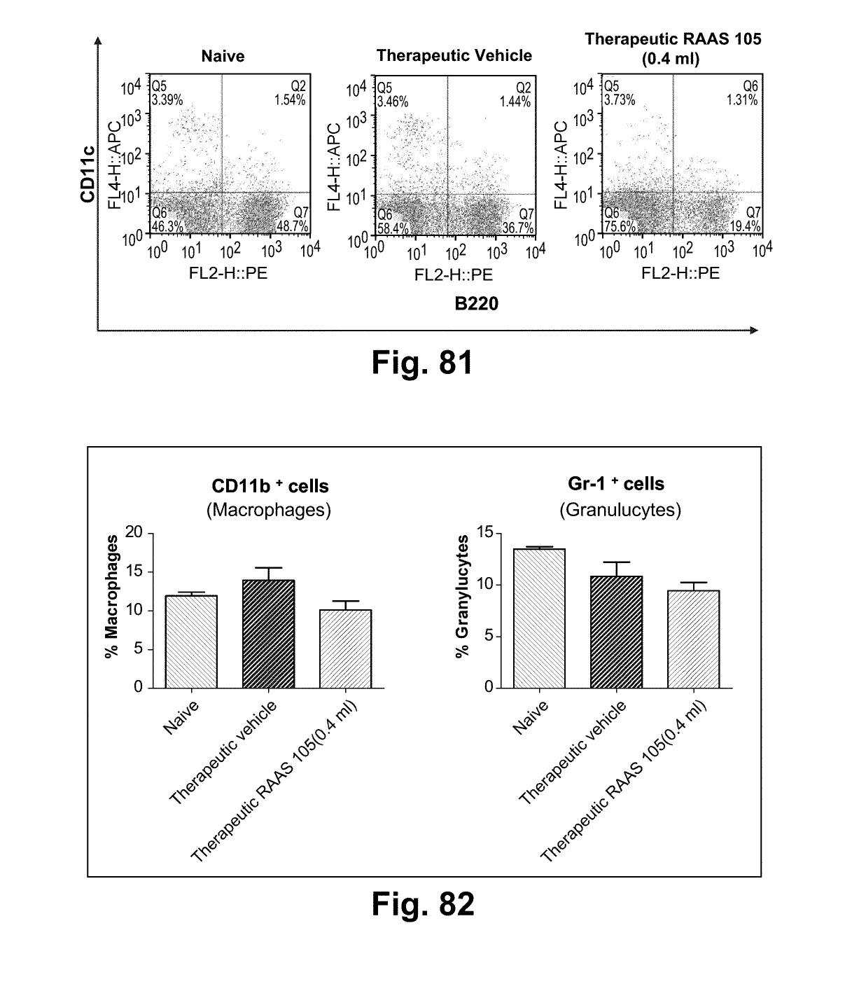

FIG. 81. Percents of mDc and pDcs in spleen. Total live cells were gated.

CD11b⁺ macrophages and Gr-1⁺ granulocytes in spleen were analyzed. There were no significant alterations among all groups in the percents of these cell lineages in spleen, as shown in FIG. 21. The representative FACS profiles from each group were illustrated in FIG. 22.

FIG. 82. Percents of Macrophages and Granulocytes in Spleen. Total live cells were gated. There were no significant differences after compound treatment. (by t test)

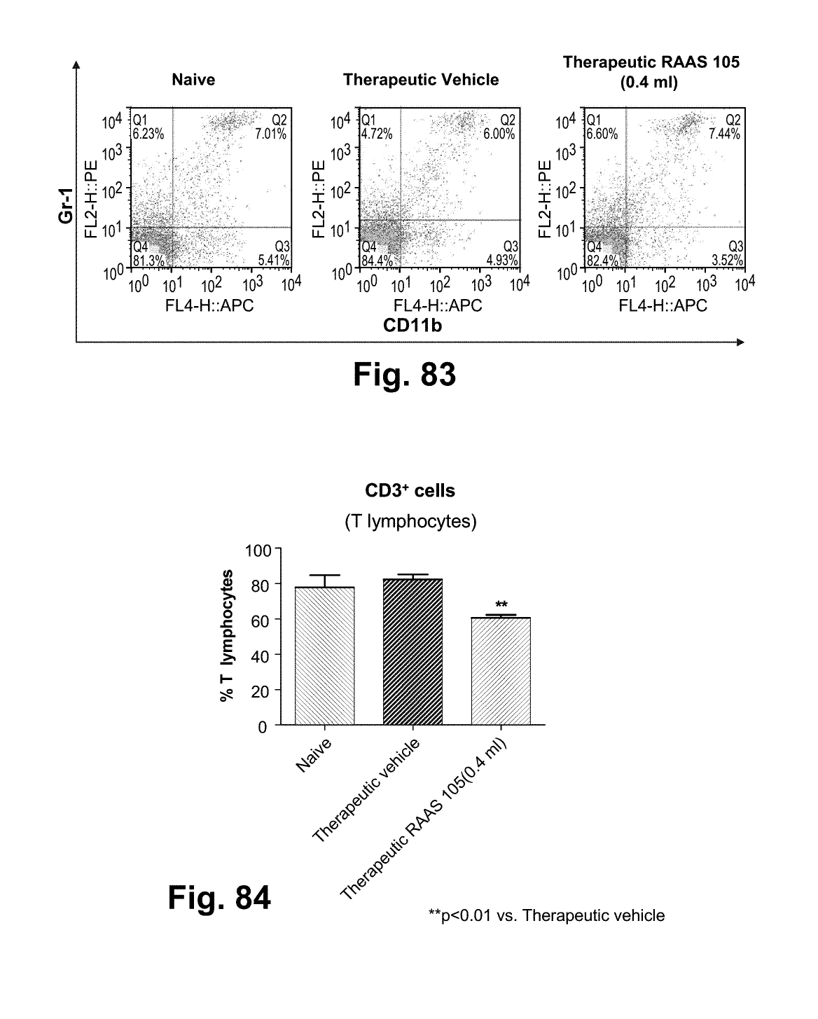

FIG. 83. Percents of macrophages/Granulocytes in spleen. Total live cells were gated.

4) Cell Populations in Draining Lymph Nodes

Cell lineages in draining lymph nodes including T cell lineages (CD4⁺/CD8⁺ T cells, naïve T cells, memory T cells and regulatory T cells), DCs, granulocytes and macrophages were characterized by cell surface and intracellular markers.

Percents of total T cells in lymph nodes were analyzed. HBV infection did not affect the percents of CD3⁺ T cells but therapeutic treatment of RAAS 105 reduced it significantly compared with vehicle group (FIG. 23). The representative FACS profiles from each group were illustrated in FIG. 24.

FIG. 84. Percents of T cells in lymph nodes. Total lymphocytes were gated. After the treatment, the percentage of T cells in the lymph nodes were significantly decreased (t test)

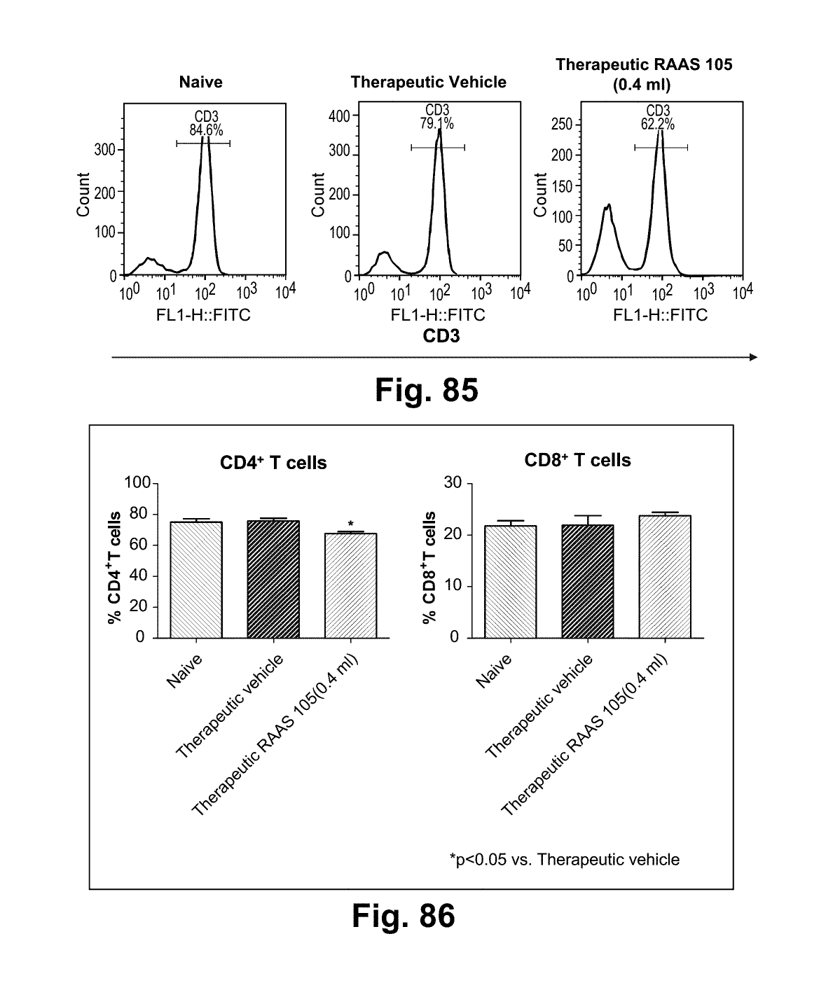

FIG. 85. Percents of CD3 T cells in lymph nodes. Total lymphocytes were gated.

Further analysis of the percents of CD4⁺ and CD8⁺T cell lineages were performed gating on total CD3⁺ T cells. Percents of CD4⁺ T cells tended to decrease while CD8⁺ T cells tended to increase, suggesting that therapeutic treatment of RAAS 105 may have effect on the ratio of CD4⁺/CD8⁺ T cells in lymph nodes (FIG. 25). The representative FACS profiles from each group were illustrated in FIG. 26.

FIG. 86. Percents of CD4 and CD8 T cells in lymph nodes. Total CD3 T cells were gated and further analyzed for CD4/CD8 percentages. After therapeutic treatment, the percentage of CD4 T cells decreased. (by t test)

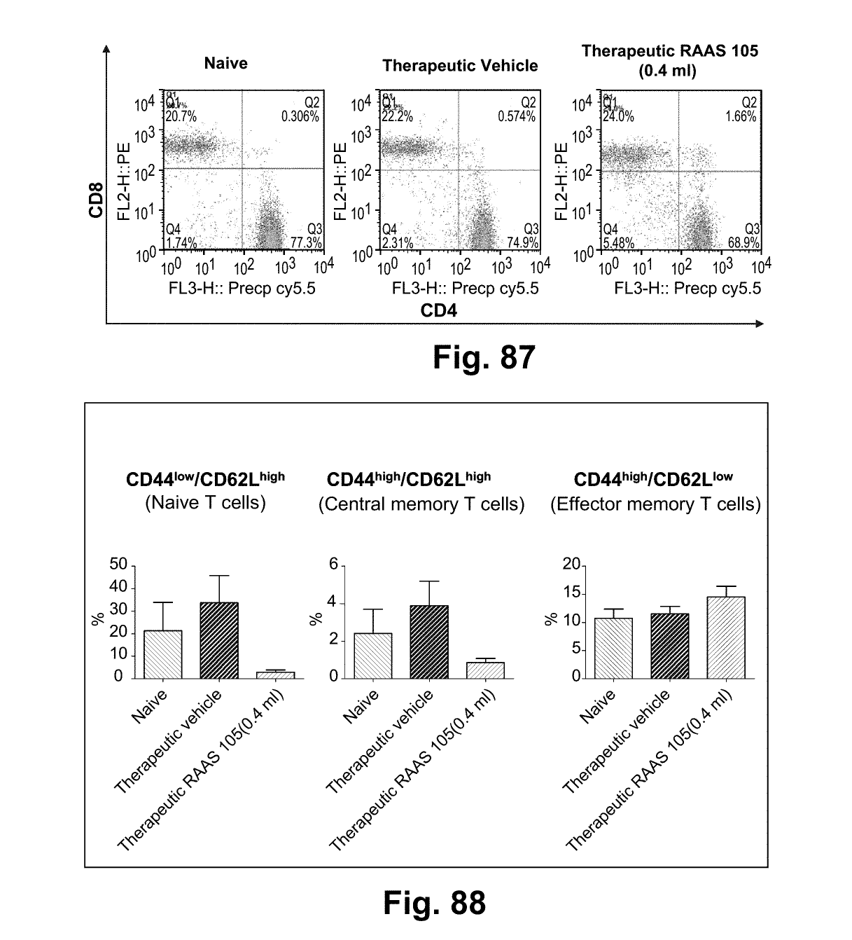

FIG. 87. Percents of CD4 and CD8 T cells in lymph nodes. Total CD3 T cells were gated and further analyzed for CD4/CD8 percentages.

Three T cell lineages, naïve T cells, T_{CM}s and T_{EM}s were characterized by surface markers CD44 and CD62L. Percents of these T cell lineages in CD4⁺ or CD8⁺ T cells were analyzed respectively. The results in lymph nodes were comparable to those in spleen. Both in CD4⁺ and CD8⁺ T cells, percents of naïve T cells and T_{CM}s decreased and T_{EM}s increased after the therapeutic treatment of RAAS 105, suggesting the compound also have effect to promote the transformation of T cells from naïve T cells to memory T cells in lymph nodes (FIGS. 27 and 29). The representative FACS profiles from each group were illustrated in FIGS. 28 and 30.

FIG. 88. CD4 T cell subsets percentages in lymph nodes. Total CD4 T cells were gated and T cell subsets were determined. No significant differences were found in all the groups compared to vehicle group.

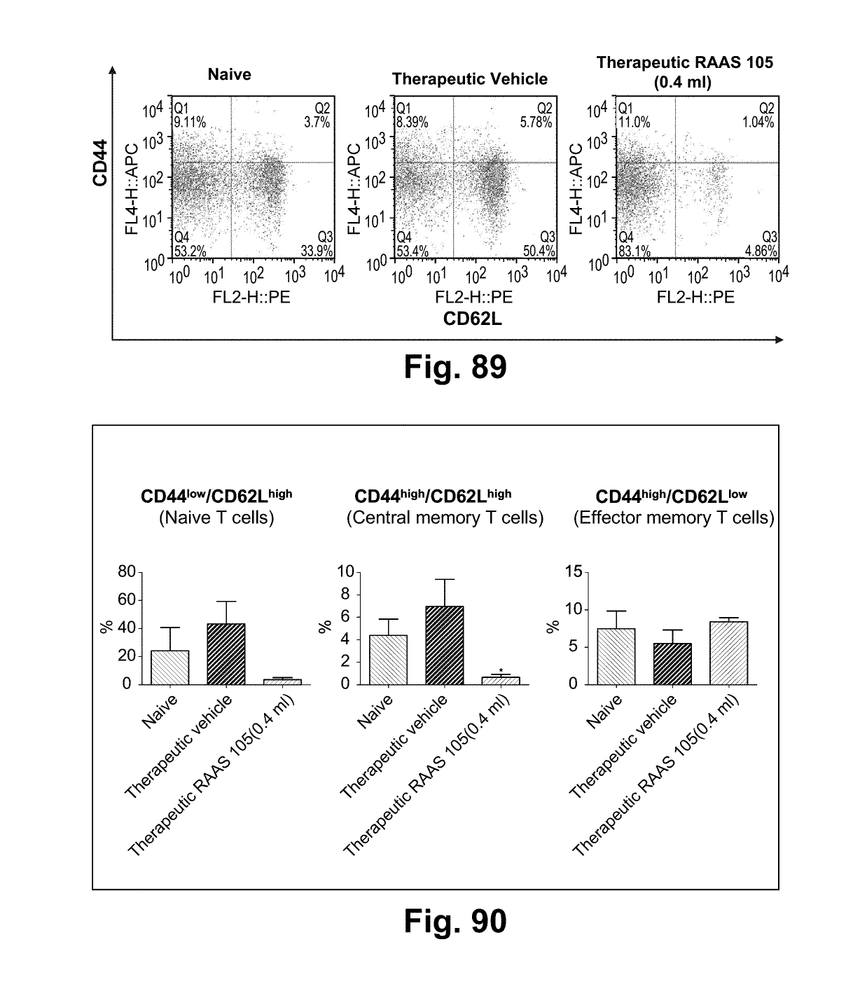

FIG. 89. CD4 T cell subset percents in lymph nodes. Total CD4 T cells were gated and T cell subsets were determined.

FIG. 90. CD8 T cell subsets percents in lymph nodes. Total CD8 T cells were gated and T cell subsets were determined.

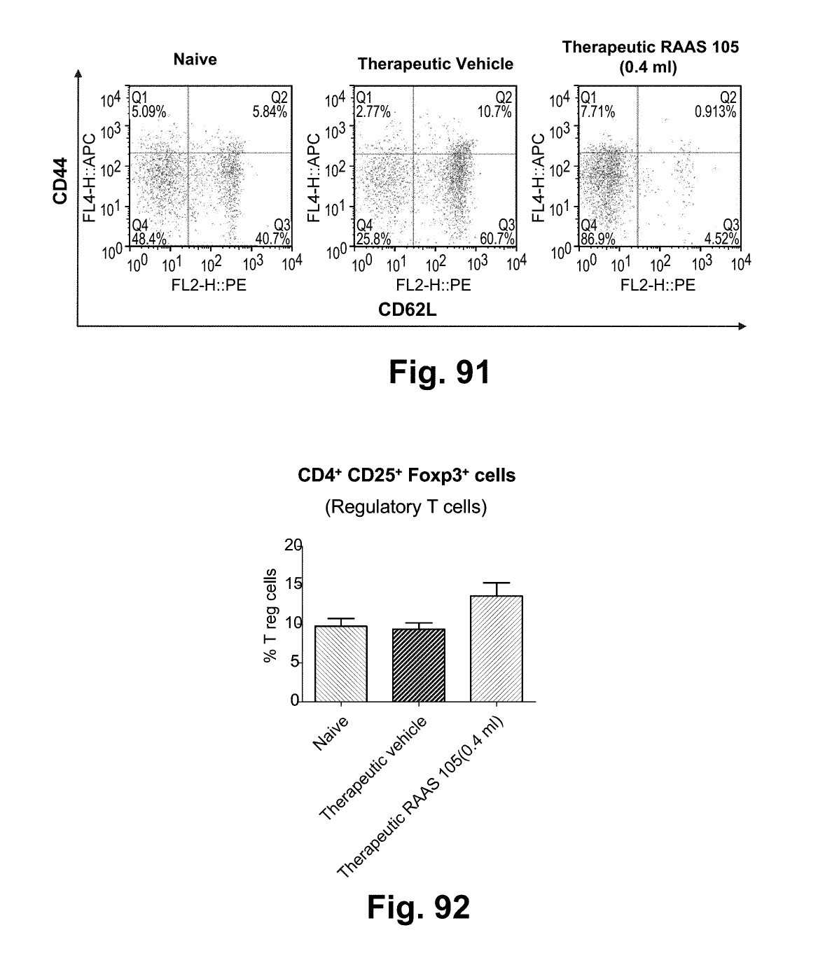

FIG. 91. CD8 T cell subsets percents in lymph nodes. Total CD8 T cells were gated and T cell subsets were determined.

Regulatory T cells (Tregs) were analyzed. Percents of Tregs in lymph node slightly increased without significant differences (FIG. 30B). The representative FACS profiles from each group were illustrated in FIG. 32.

FIG. 92. Percents of Foxp3 regulatory T cells in lymph nodes. There were no significant alterations after compound treatment

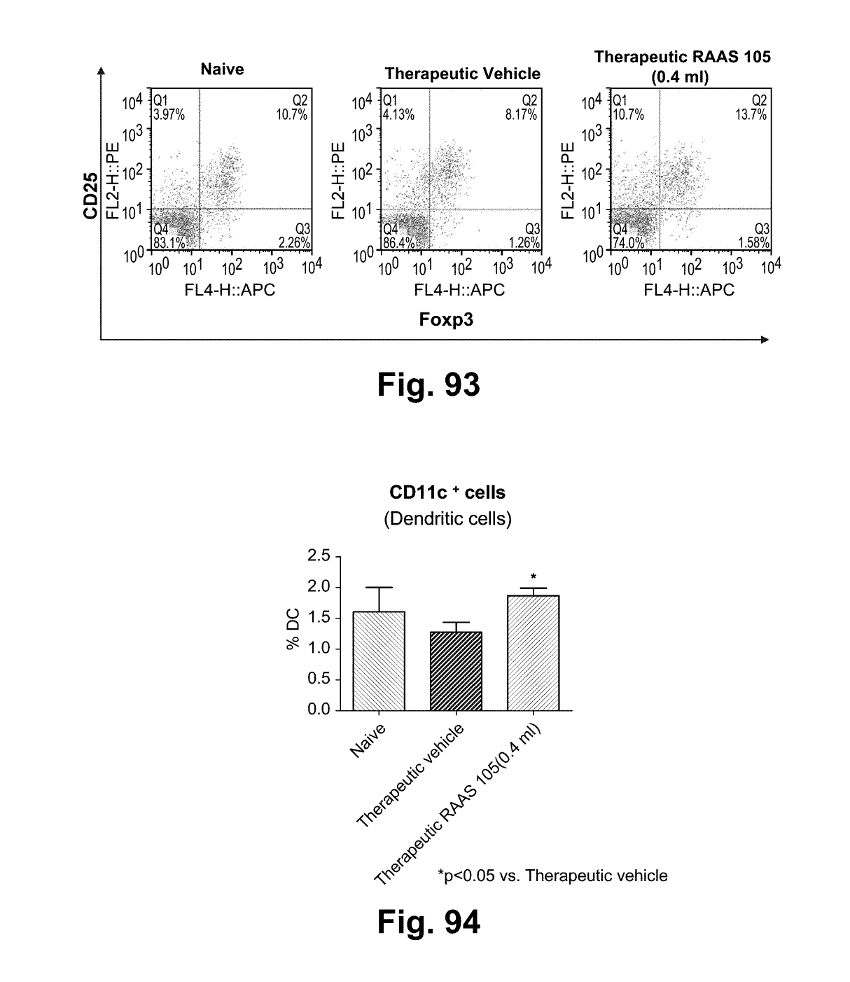

FIG. 93. Percents of regulatory T cells in lymph nodes. Total CD4 T cells were gated. One representative profile from each group is shown.

Total dendritic cells in lymph nodes were analyzed. Therapeutic treatment of RAAS 105 may reverse the reduction of DCs induced by HBV infection (FIG. 33). The representative FACS profiles from each group were illustrated in FIG. 34.

FIG. 94. Percents of DCs in lymph nodes. Total live cells were gated. After treatment, percents of DCs increased significantly (by t test)

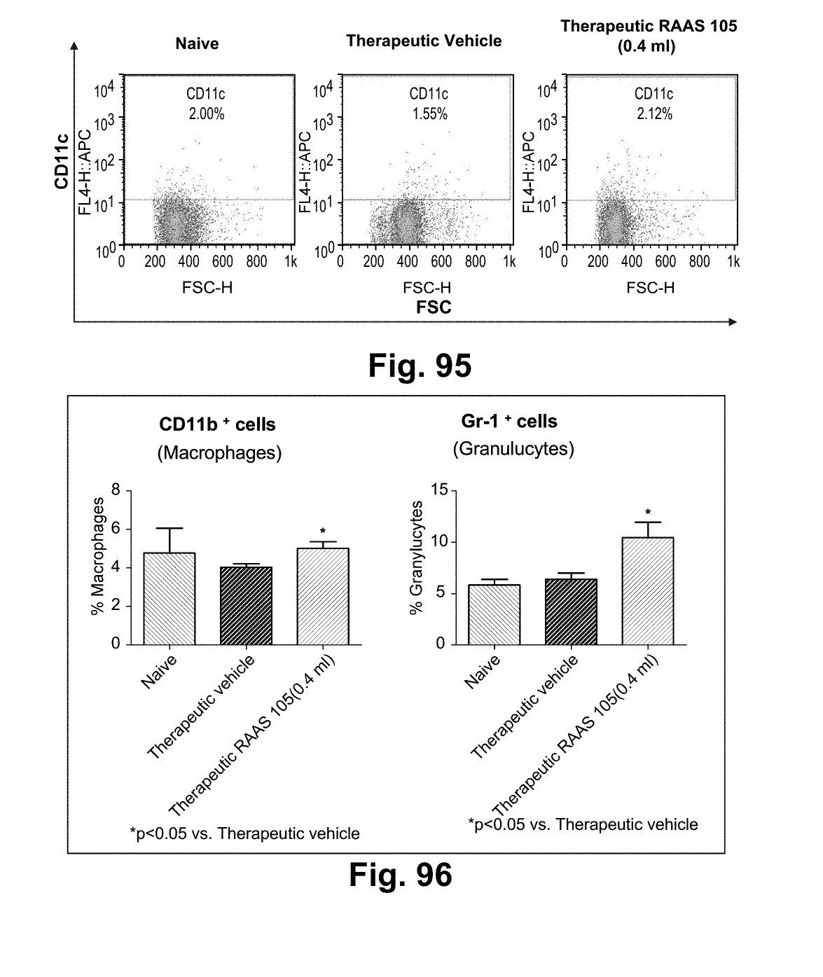

FIG. 95. Percents of DCs in lymph nodes. Total live cells were gated.

CD11b⁺ macrophages and Gr-1⁺ granulocytes in lymph nodes were analyzed. Both percents of CD11b⁺ macrophages and Gr-1⁺ granulocytes increased significantly (FIG. 35). The representative FACS profiles from each group were illustrated in FIG. 36E.

FIG. 96. Percents of Macrophages and Granulocytes in lymph nodes. Total live cells were gated. Percents of macrophages and granulocytes significantly increased in lymph node. (by t test)

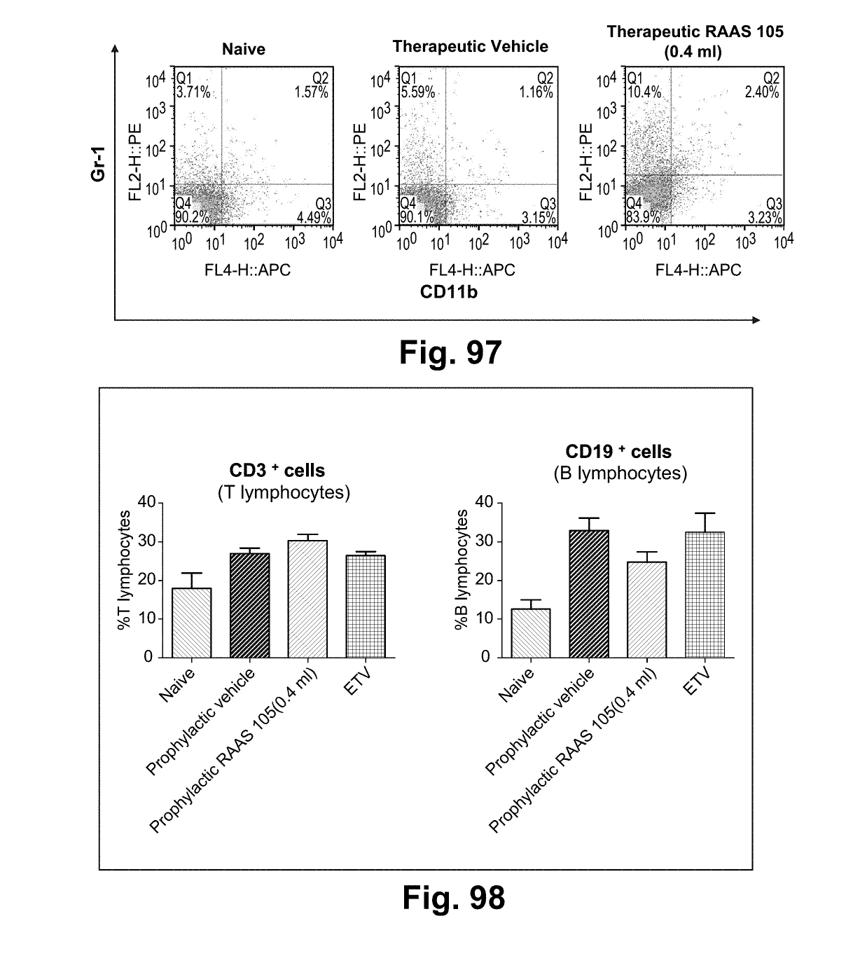

FIG. 97. Percents of Macrophages/Granulocytes in lymph nides. Total live cells were gated.

5.3.2 Effect of Prophylactic Treatment with RAAS 105

1) Mice Information

Total 14 female BALB/c mice including 2 naïve mice at the same age were transferred from Infectious Disease (ID) Group of Wuxi Apptec. The group and the regimen information were shown by Table 2.

TABLE 2
The experimental group and closing regimen of the 2^{nd} part of the study

| | | | | 1^{st} or last | |
|---|---|---|---|---|---|
| Groups | N | Group ID | Dose | dosing | Analysis |
| 5 | 4 | Prophylactic | — | last, 4 hrs | Day 5 |
| | | vehicle^{#} | | pre-HDI | |
| 7 | 4 | Prophylactic | 0.4 ml/mouse | last, 4 hrs | Day 5 |
| | | RAAS 105 | | pre-HDI | |
| 10 | 4 | ETV | 0.1 mg/kg | 1^{st}, 4 hrs | Day 5 |
| | | | | pre-HDI | |
| 11 | 2 | Naive | — | — | — |

2) Cell Populations in Peripheral Blood

After removing red blood cells, T cell lineages, B cells, DCs, granulocytes, and monocytes/macrophages in peripheral blood were analyzed by FACS analysis.

Total T cells and B cells were characterized. Unlike therapeutic treatment, prophylactic treatment of RAAS 105 had no effect on percents of CD3⁺ T cells but reduced the percents of CD19⁺B cells although the statistical significance was not found (FIG. 37). The representative FACS profiles from each group were illustrated in FIG. 38.

FIG. 98. Percents of T and B lymphocytes in peripheral blood. Total lymphocytes were gated.

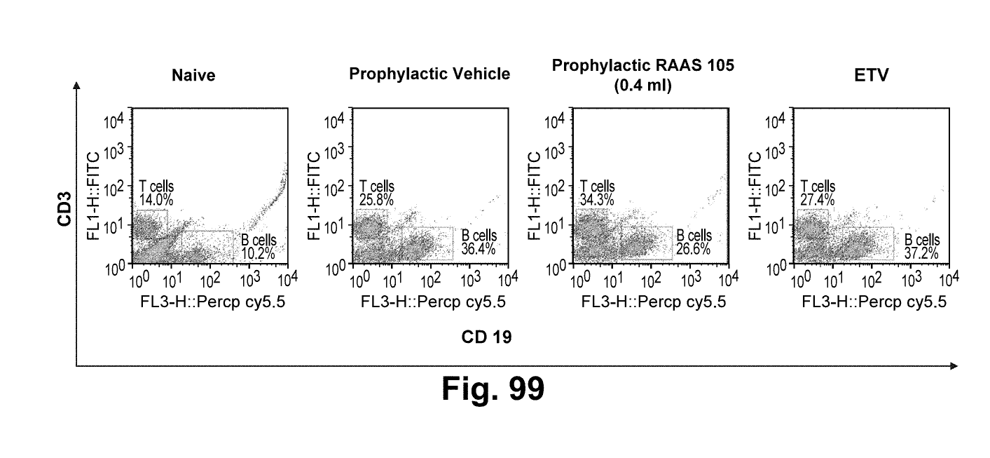

FIG. 99. Percents of T cells and B cells in peripheral blood. Total lymphocytes were gated.

Further analysis of the percents of CD4⁺ and CD8⁺ (non-CD4⁺) T cell lineages were performed gating on total CD3⁺ T cells. Unlike therapeutic treatment, prophylactic treatment reduced percents of CD4⁺ T cells and increased percents of CD8⁺ T cells, suggesting the potential effect of RAAS 105 to reduce the ratio of CD4⁺/CD8⁺ T cells in peripheral blood (FIG. 39). The representative FACS profiles from each group were illustrated in FIG. 40.

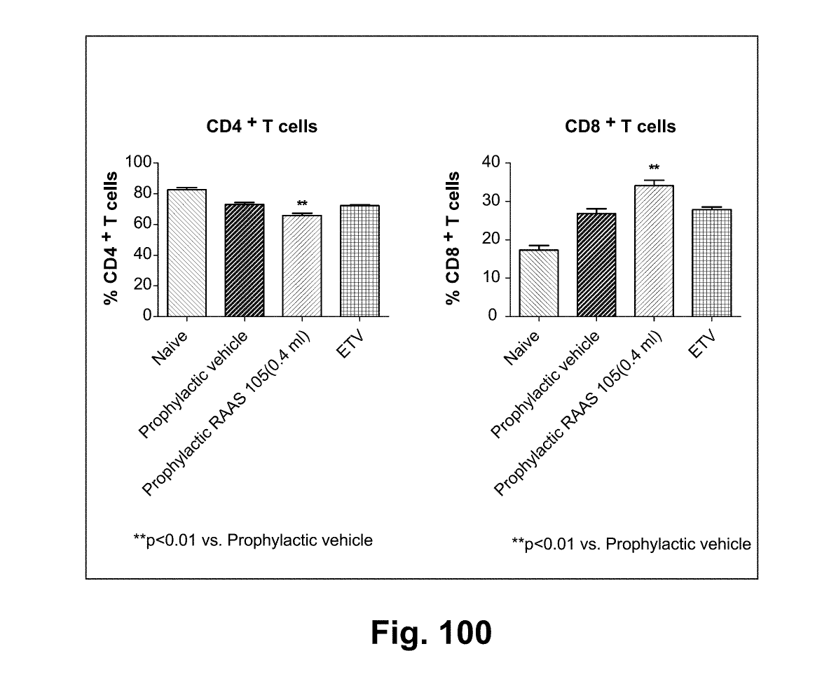

FIG. 100. Percents of CD4 and CD8 T cells in peripheral blood. Total CD3 T cells were gated and further analyzed for CD4/CD8 percentages. After prophylactic treated by RAAS 105, percents of CD4 T cells decreased while CD8 T cells increased (by t test)

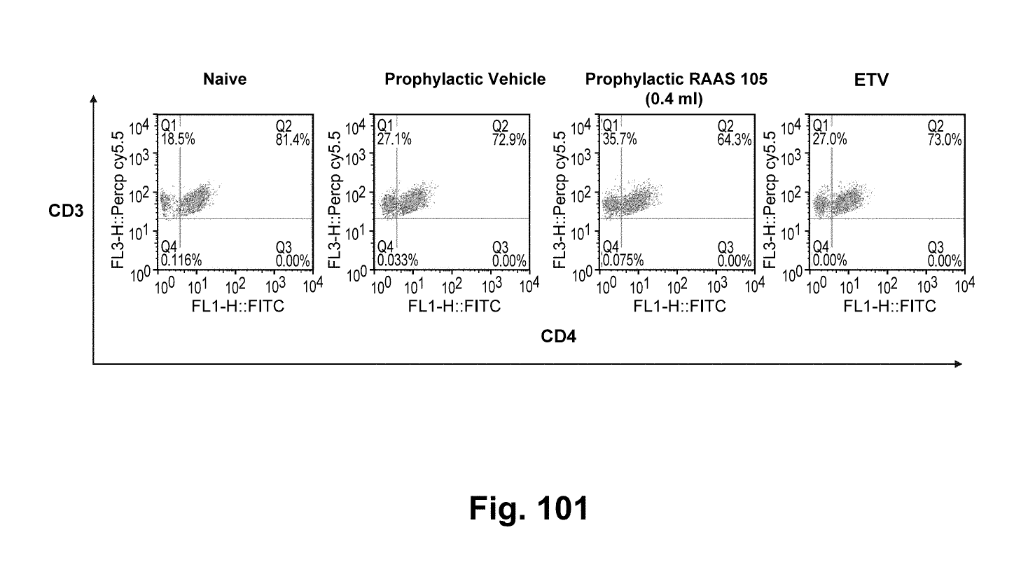

FIG. 101. Percents of CD4 and CD8 T cells in peripheral blood. Total CD3 T cells were gated.

Results of total CD11c⁺ dendritic cells (DC) and Gr-1⁺ granulocytes in peripheral blood were also different from those in therapeutic treatment. Prophylactic treatment of RAAS 105 reversed the reduction of DCs induced by HBV infection, but had no significant effect on granulocytes in peripheral blood (FIG. 41). The representative FACS profiles from each group were illustrated in FIG. 42.

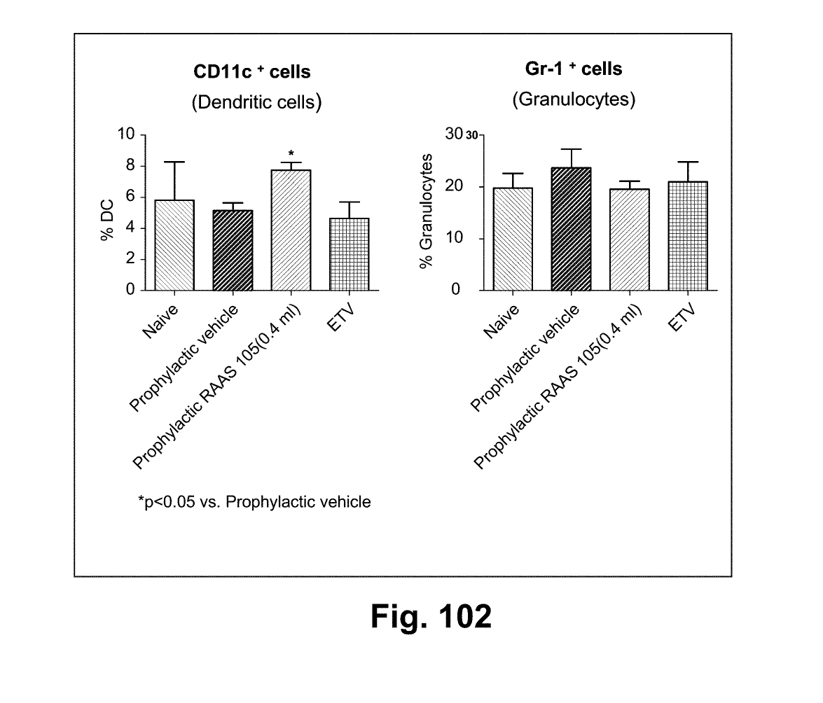

FIG. 102. Percents of Dendritic cells and Granulocytes in peripheral blood. Total live cells were gated. After prophylactic treated, percents of dendritic cells increased in peripheral blood.

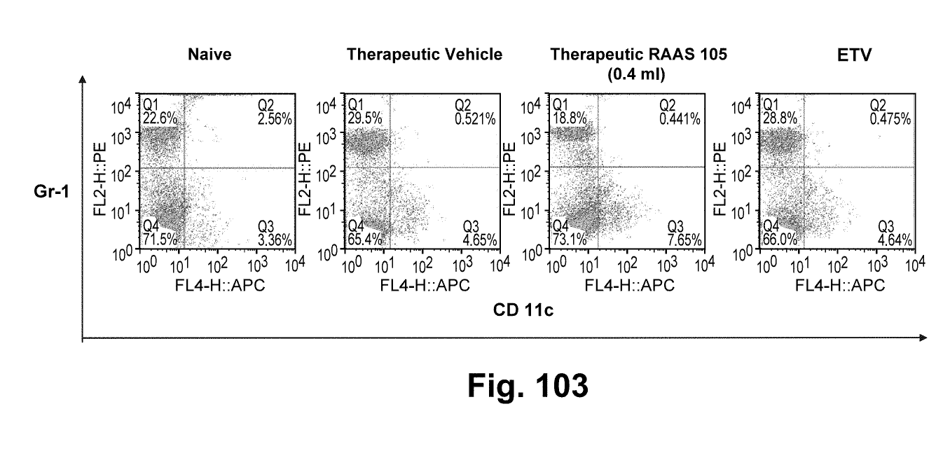

FIG. 103. Percents of Granulocytes/Dendritic cells in peripheral blood. Total live cells were gated.

Percents of Monocytes were examined. There were no significant differences among all groups (FIG. 43). The representative FACS profiles from each group were illustrated in FIG. 44.

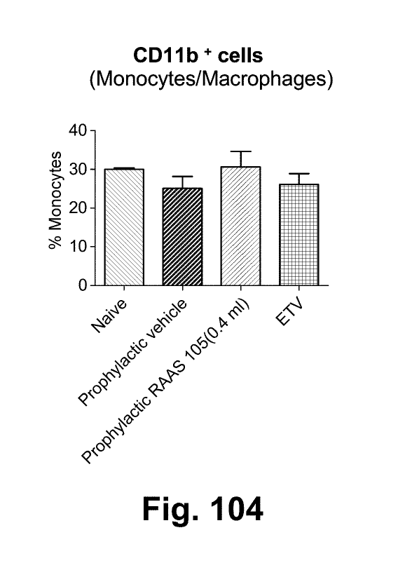

FIG. 104. Percents of Monocytes in peripheral blood. Total live cells were gated.

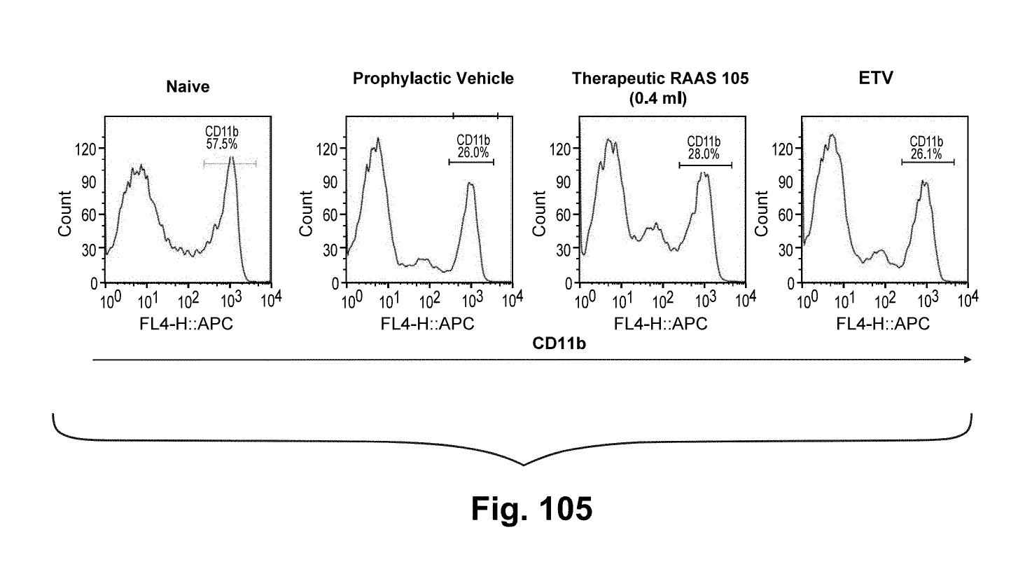

FIG. 105. Percents of monocytes in peripheral blood. Total live cells were gated.

3) Cell Populations in Spleen

Cell lineages in spleen including T cell lineages (CD4⁺/CD8⁺ T cells, naïve T cells, memory T cells and regulatory T cells), B cells, mDCs, pDCs, granulocytes and macrophages were characterized by cell surface and intracellular markers.

Percents of total T cells and B cells in spleen were investigated. Unlike therapeutic treatment, prophylactic treatment did not show effects on percents of CD3⁺ T cells and CD19⁺B cells (FIG. 45). The representative FACS profiles from each group were illustrated in FIG. 46.

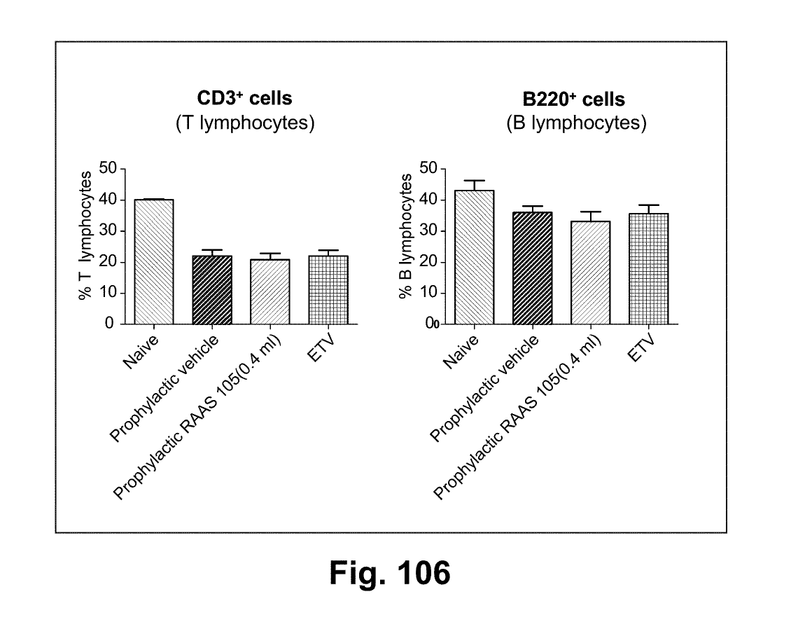

FIG. 106. Percents of T and B lymphocytes in spleen. Total lymphocytes were gated.

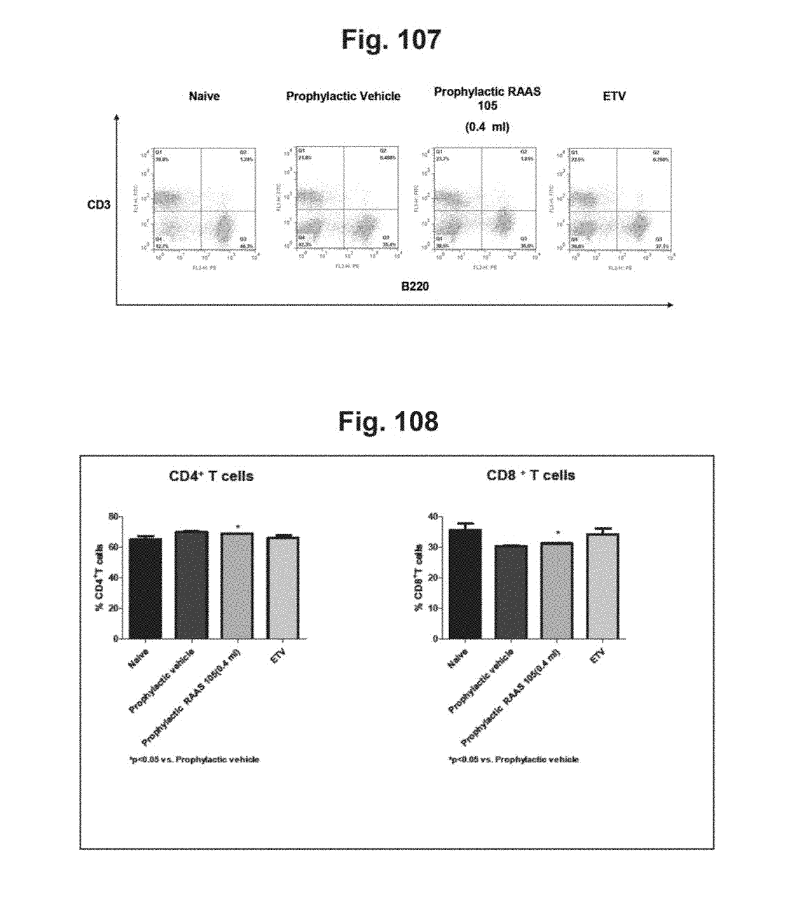

FIG. 107. Percents of T and B cells in spleen. Total lymphocytes were gated.

Further analysis of the percents of CD4⁺ (non-CD8⁺) and CD8⁺T cell lineages were performed gating on total CD3⁺ T cells. Percents of CD4⁺ T cells slightly decreased and CD8⁺T cells slightly increased in spleen (FIG. 47). The representative FACS profiles from each group were illustrated in FIG. 48.

FIG. 108. Percents of CD4 and CD8 T cells in spleen. Total CD3 T cells were gated and further analyzed for CD4/CD8 percentages. After prophylactic treated by RAAS 105, the percentage of CD4 T cells slightly decreased while CD8 T cells slightly increased (by t test)

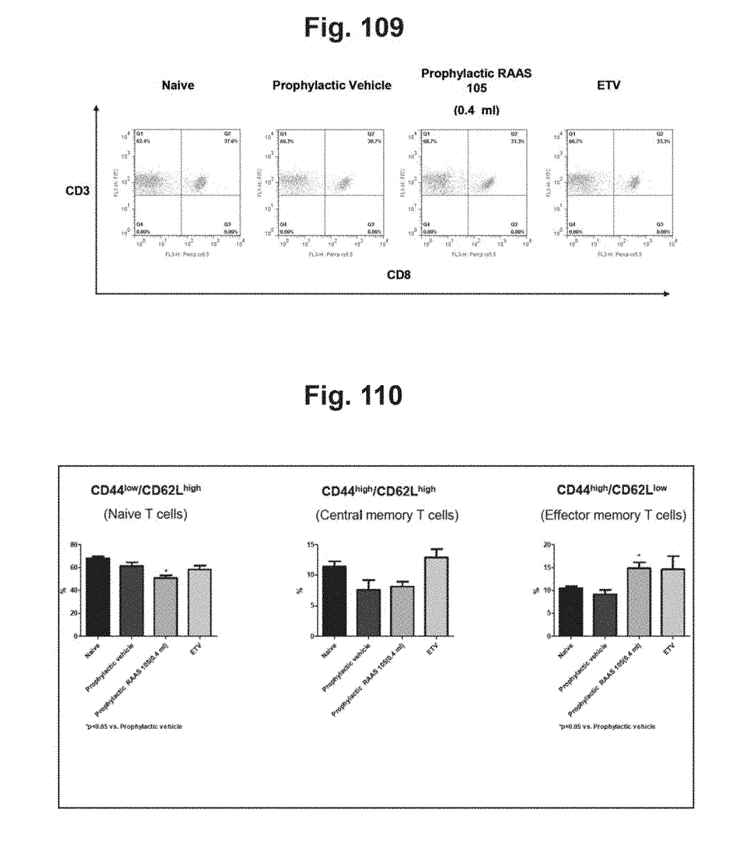

FIG. 109. Percents of CD4 and CD8 T cells in spleen. Total CD3 T cells were gated and further analyzed for CD4/CD8 percentages.

Naïve T cells, central memory T cells and Effector memory T cells were investigated. Percents of these T cell lineages in CD4⁺ or CD8⁺ T cells in spleen were analyzed respectively. Both in CD4⁺ and CD8⁺ T cells, percents of naïve T cells decreased and T_{EM}s increased significantly after the prophylactic treatment of RAAS 105 (FIGS. 49 and 51). The representative FACS profiles from each group were illustrated in FIGS. 50 and 52.

FIG. 110. T cell subset percents in spleen. Total CD4 T cells were gated and T cell subsets were determined.

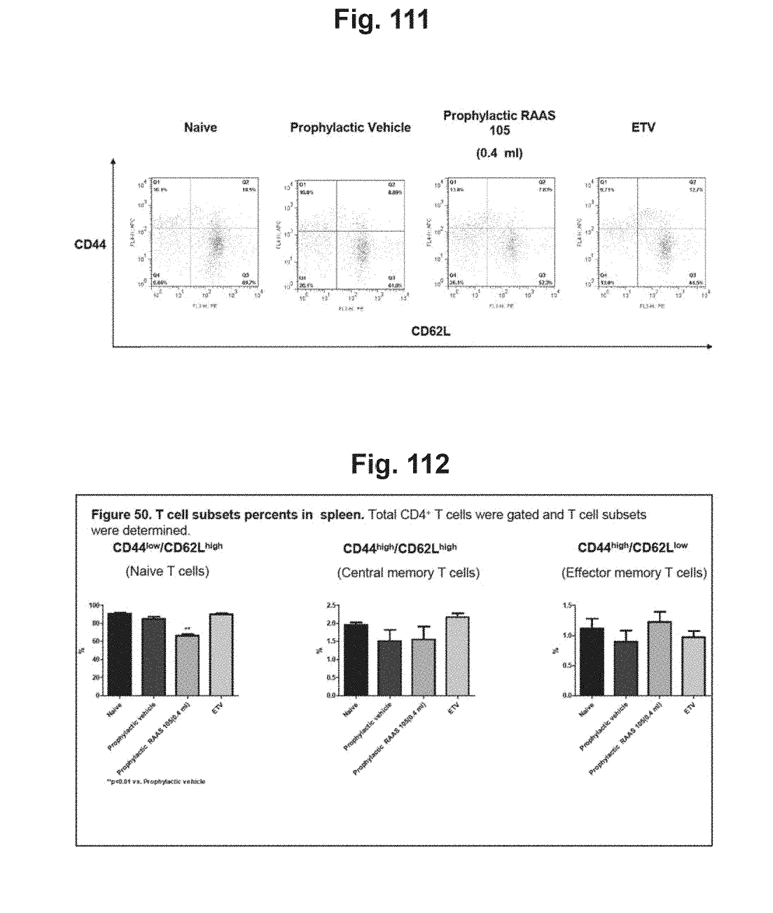

FIG. 111. T Cell subsets percents in spleen. Total CD4 T cells were gated and T cell subsets were determined.

FIG. 112. T cell subsets percents in spleen. Total CD8 T cells were gated and T cell subsets were determined.

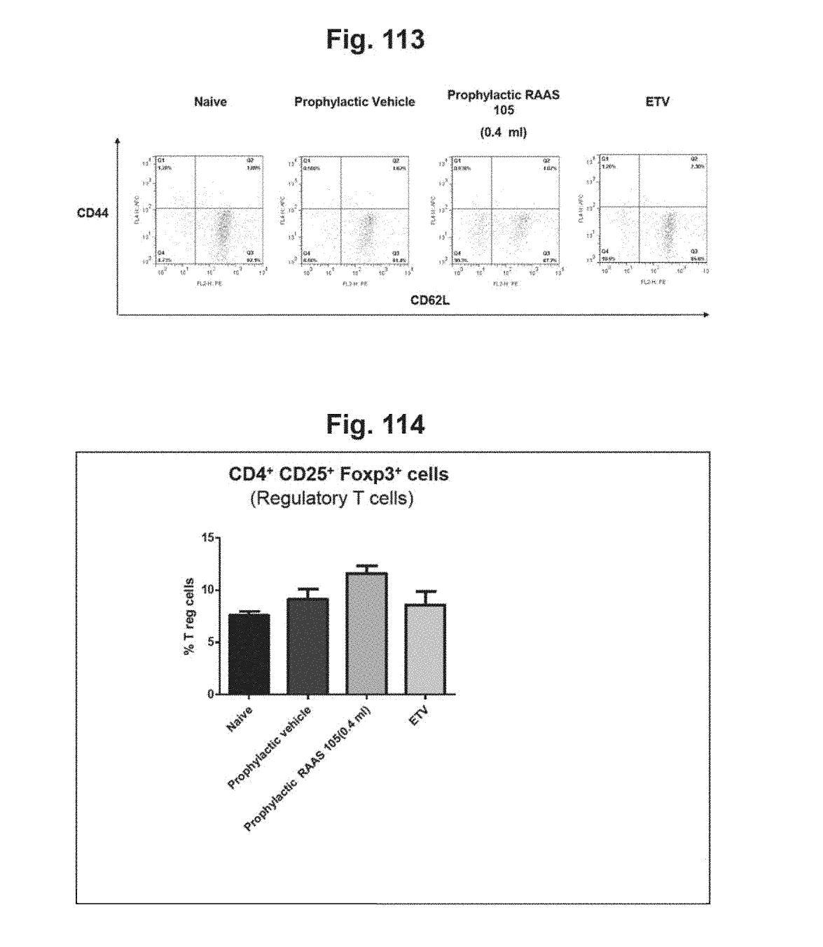

FIG. 113. T cell subsets percents in spleen. Total CD8 T cells were gated and T cell subsets were determined.

Results of regulatory T cells (Tregs) were comparable with those in therapeutic treatment. Percents of Tregs in spleen increased compared with the vehicle group by prophylactic treatment of RAAS 105 (FIG. 53). The representative FACS profiles from each group were illustrated in FIG. 54.

FIG. 114. Percents of Foxp3 regulatory T cells in spleen. Foxp3 regulatory T cells were analyzed by intracellular staining

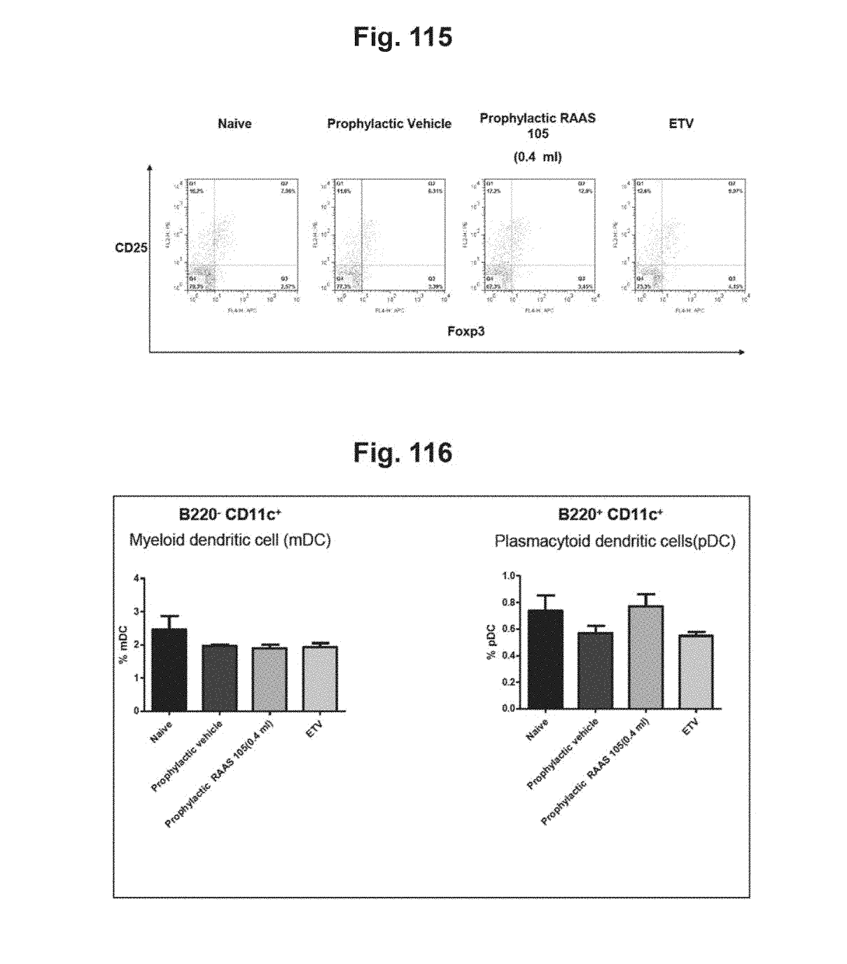

FIG. 115. Percents of regulatory T cells in spleen. Total CD4 T cells were gated.

Dendritic cells, including mDCs and pDCs in spleen were analyzed. No significant differences of mDCs and pDCs were observed among all groups after prophylactic treatment (FIG. 55). The representative FACS profiles from each group were illustrated in FIG. 56.

FIG. 116. Percents of pDCs and mDC in spleen. Total live cells were gated. There were no significant differences after compound treatment (by t test)

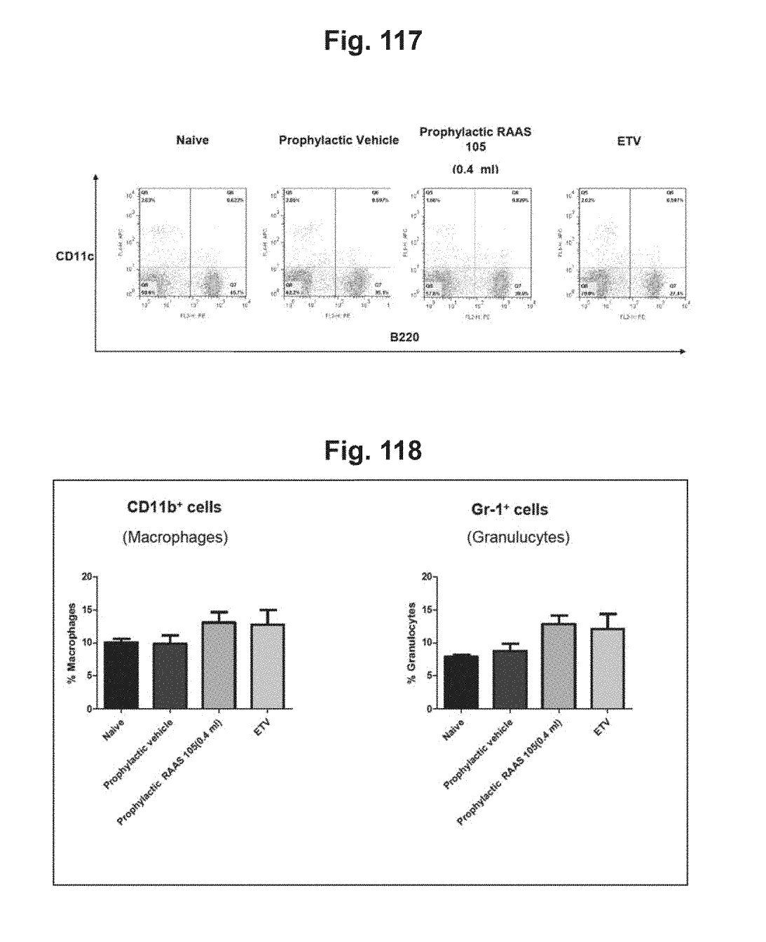

FIG. 117. Percents of mDCs and pDCs in spleen. Total live cells were gated.

CD11b⁺ macrophages and Gr-1⁺ granulocytes in spleen were analyzed. Percents of macrophages and granulocytes increased, but no statistical differences were observed, as shown in FIG. 57. The representative FACS profiles from each group were illustrated in FIG. 58.

FIG. 118. Percents of Macrophages and Granulocytes in spleen. Total live cells were gated. There were no significant differences after compound treatment. (by t test)

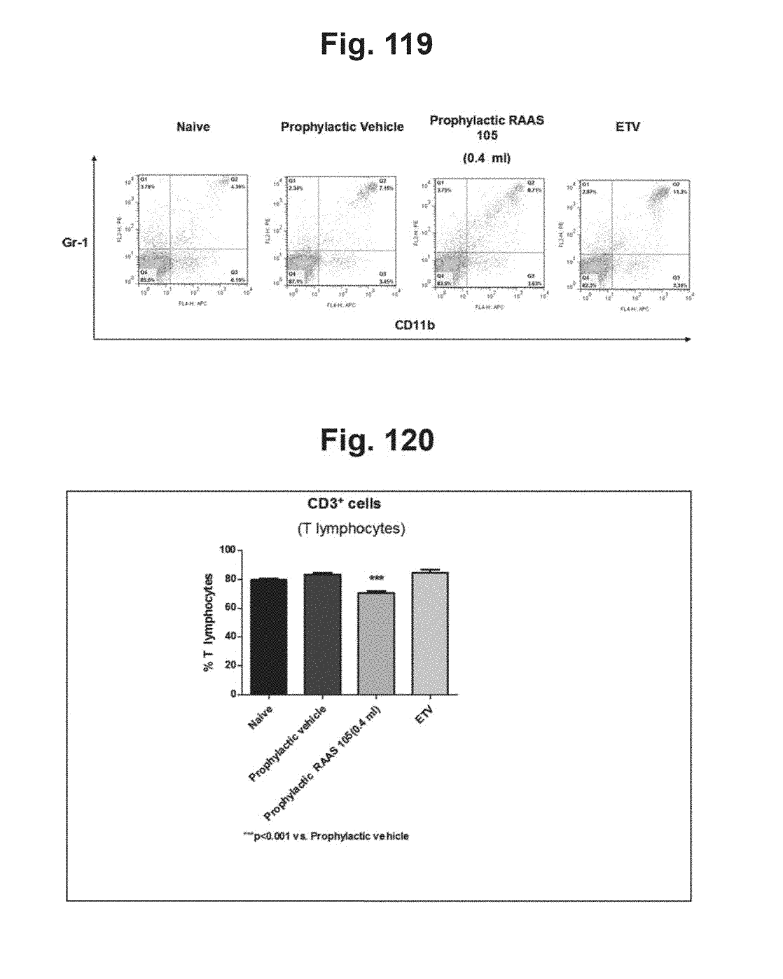

FIG. 119. Percents of macrophages/granulocytes in spleen. Total live cells were gated.

4) Cell Populations in Draining Lymph Nodes

Cell lineages in draining lymph nodes including T cell lineages (CD4⁺/CD8⁺ T cells, naïve T cells, memory T cells and regulatory T cells), DCs, granulocytes and macrophages were characterized by cell surface and intracellular markers.

Percents of total T cells in lymph nodes were analyzed. Similar with therapeutic treatment, HBV infection did not affect the percents of CD3⁺ T cells but prophylactic treatment of RAAS 105 reduced it significantly compared with vehicle group (FIG. 59). The representative FACS profiles from each group were illustrated in FIG. 60.

FIG. 120. Percents of T cells in lymph nodes. Total lymphocytes were gated. After the treatment, percents of T cells in the lymph nodes were significantly decreased. (t test)

FIG. 121. Percents of CD3 T cells in lymph nodes. Total lymphocytes were gated.

Further analysis of the percents of CD4⁺ and CD8⁺T cell lineages were performed gating on total CD3⁺ T cells. Percents of CD4⁺ T cells tended to decrease while CD8⁺ T cells tended to increase after prophylactic treatment, as was seen in therapeutic treatment (FIG. 61). The representative FACS profiles from each group were illustrated in FIG. 62.

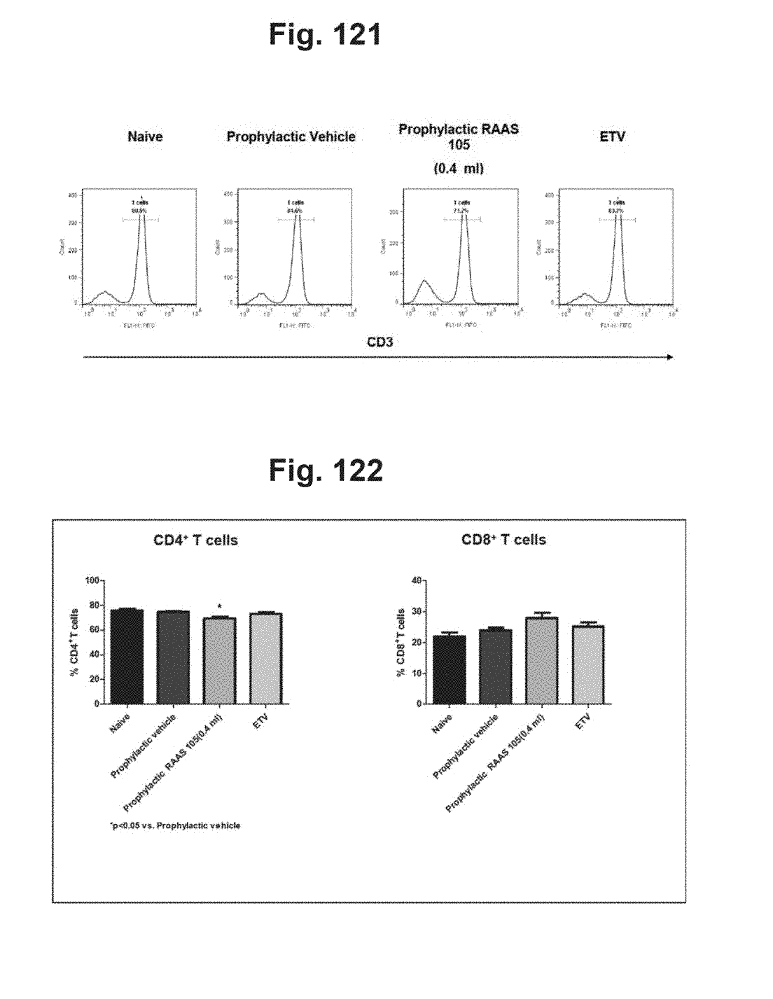

FIG. 122. Percents of CD4 and CD8 T cells in lymph nodes. Total CD3 T cells were gated and further analyzed for CD4/CD8 percentages. After prophylactic treatment, percents of CD4 T cells decreased (by t test)

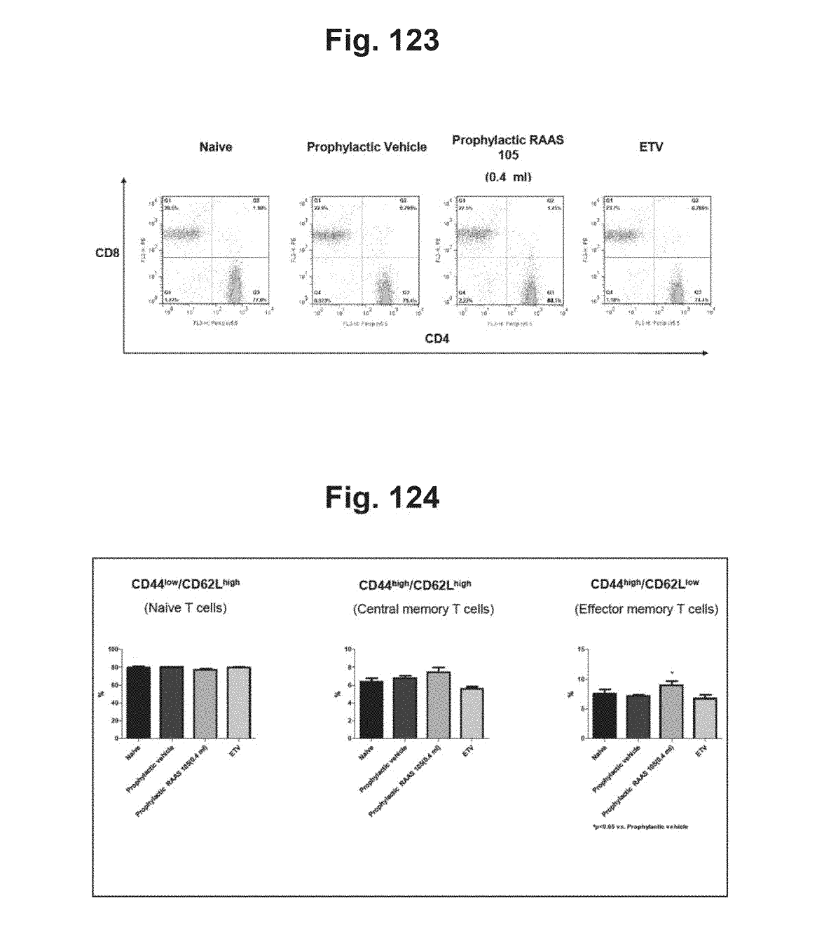

FIG. 123. Percents of CD4 and CD8 T cells in lymph nodes. Total CD3 T cells were gated and further analyzed for CD4/CD8 percentages.

Results of naïve T cells, central memory T cells and Effector memory T cells were totally difference with those in therapeutic treatment. Prophylactic treatment did not show significant effects on naïve T cells and T_{CM}s, but increased percents of T_{EM}s (FIGS. 63 and 65). The representative FACS profiles from each group were illustrated in FIGS. 64 and 66.

FIG. 124. T cell subsets percents in lymph nodes. Total CD4 cells were gated and T cell subsets were determined. No significant differences were found except effector memory T cells compared to vehicle group.

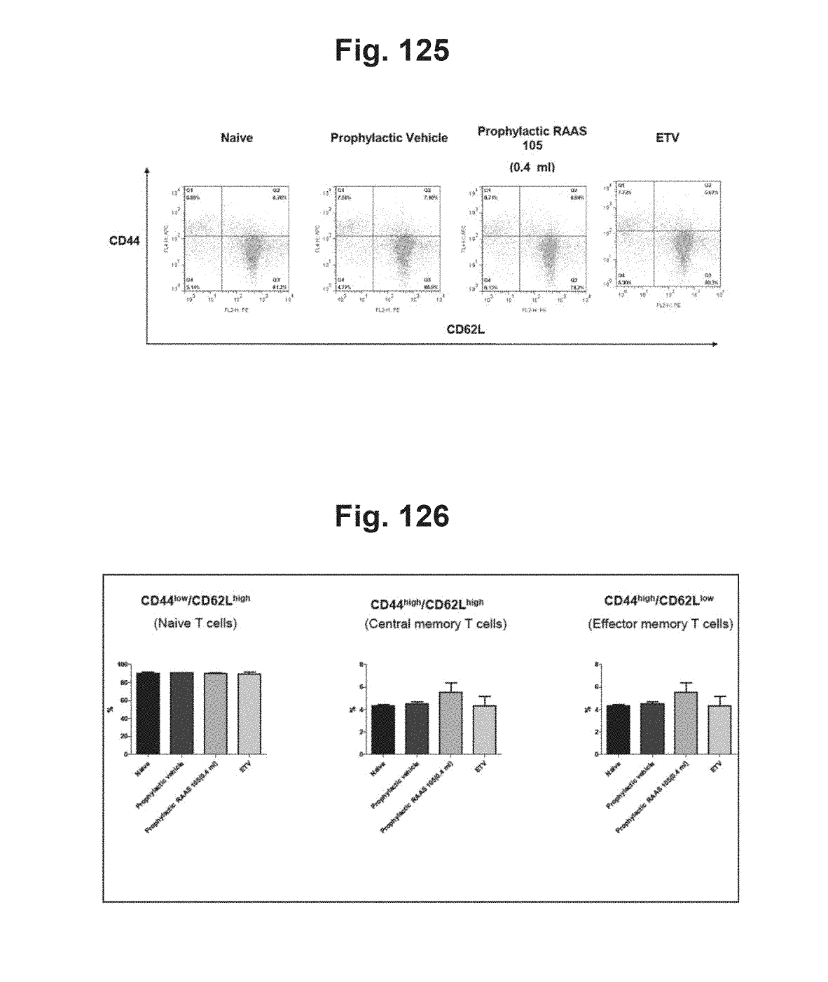

FIG. 125. T cell subsets percents in lymph nodes. Total CD4 T cells were gated and T cell subsets were determined.

FIG. 126. T cell subsets percents in lymph nodes. Total CD8 T cells were gated and T cell subsets were determined. No significant differences were found in all the groups compared to vehicle group.

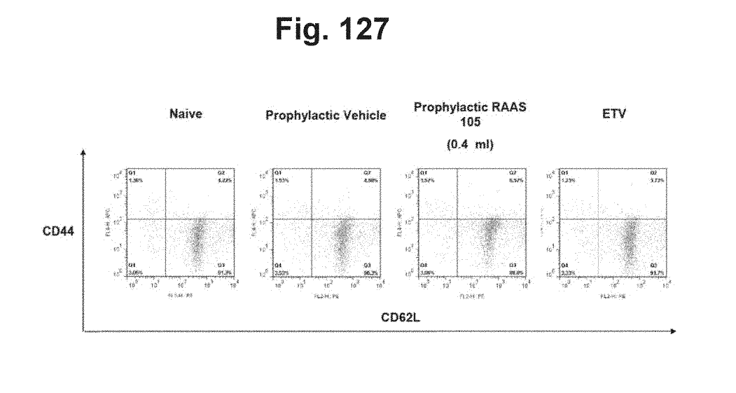

FIG. 127. T cell subsets percents in lymph nodes. Total CD8 T cells were gated and T cell subsets were determined.

Regulatory T cells were analyzed. There were no significant differences among all groups (FIG. 67). The representative FACS profiles from each group were illustrated in FIG. 68.

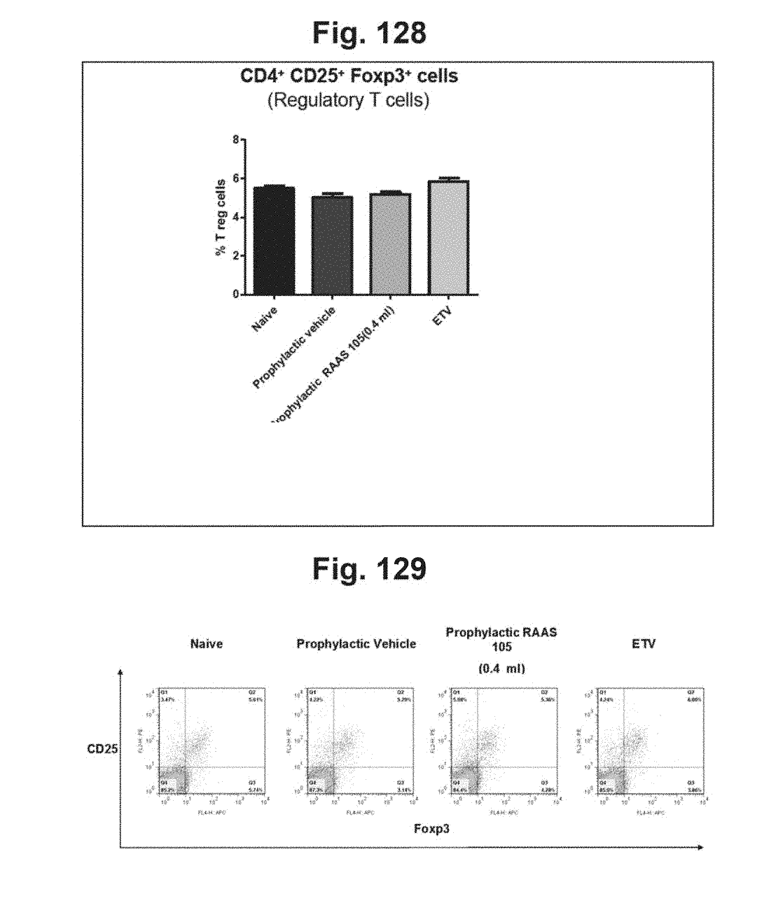

FIG. 128. Percents of Foxp3 regulatory T cells in lymph nodes. Foxp3 regulatory T cells were analyzed by intracellular staining There were no significant alterations after compound treatment. (by t test)

FIG. 129. Percents of regulatory T cells in lymph nodes. Total CD4 T cells were gated.

Results of total dendritic cells in lymph nodes were similar with those in therapeutic treatment. Prophylactic treatment of RAAS 105 also increased the percents of DCs significantly compared with vehicle group (FIG. 69). The representative FACS profiles from each group were illustrated in FIG. 70.

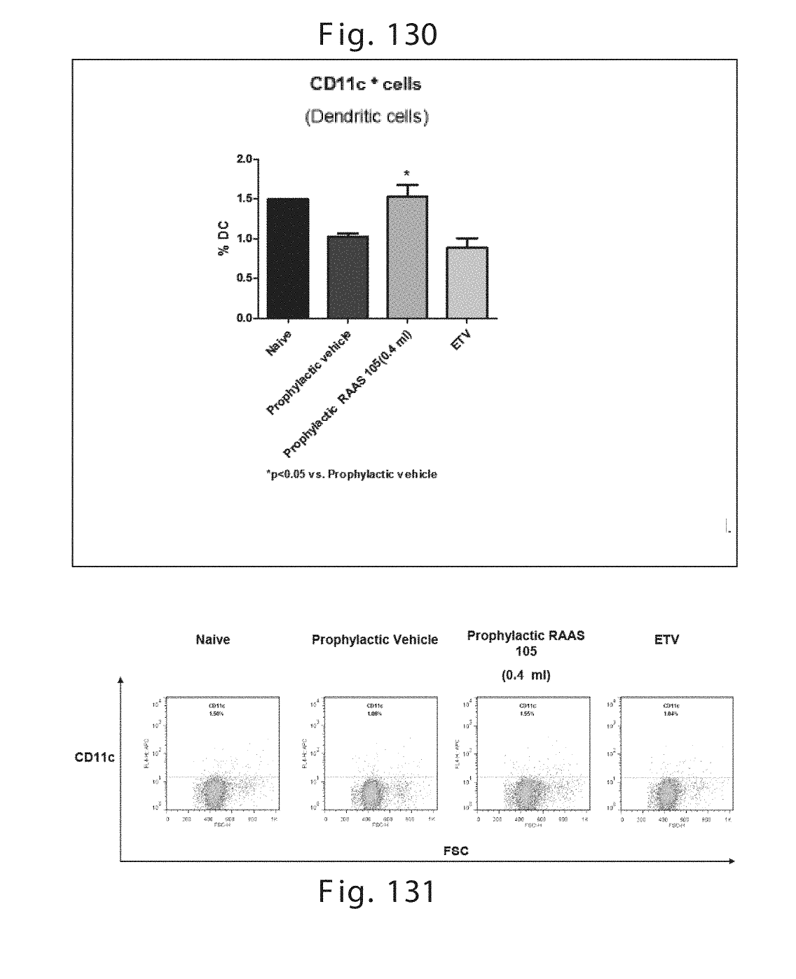

FIG. 130. Percents of DCs in lymph nodes. Total live cells were gated. After the treatment, percents of the DCs increased significantly (by t test)

FIG. 131. Percents of DCs in lymph nodes. Total live cells were gated.

CD11b⁺ macrophages and Gr-1⁺ granulocytes in lymph nodes were analyzed. Both macrophages and granulocytes increased significantly (FIG. 71). The representative FACS profiles from each group were illustrated in FIG. 72.

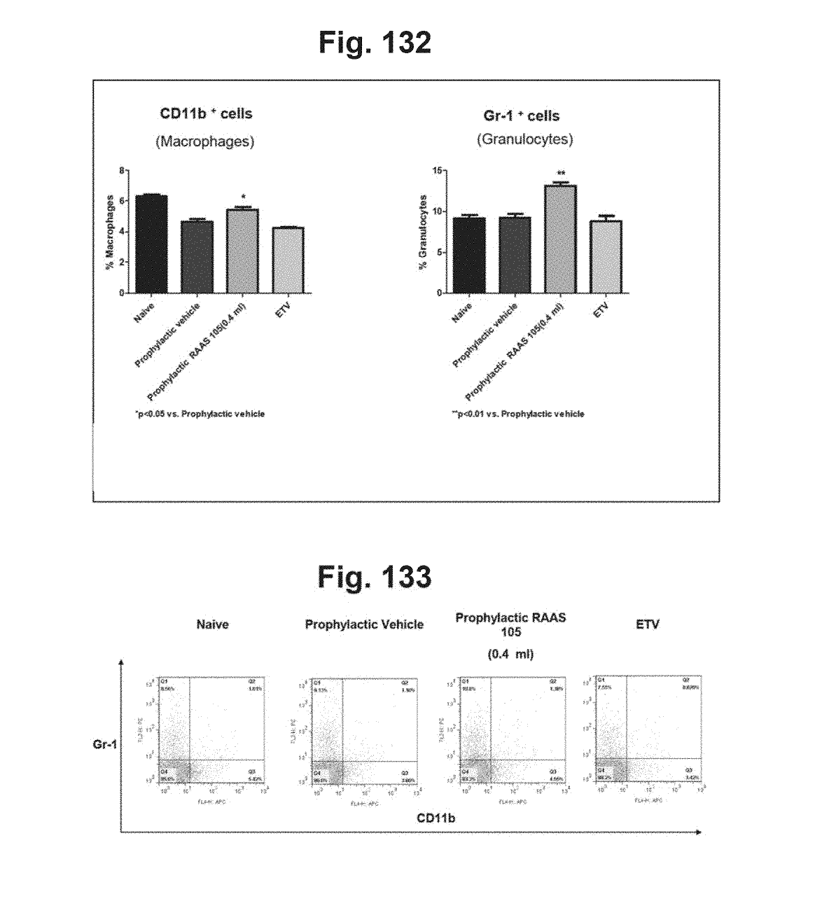

FIG. 132. Percents of Macrophages and Granulocytes in lymph nodes. Total live cells were gated. After therapeutic treated by RAAS 105, percents of macrophages and granulocytes significantly increased. (by t test)

FIG. 133. Percents of Macrophages/Granulocytes in lymph nodes. Total live cells were gated.

7 Conclusions

The effects of RAAS 105 on different cell lineages in lymphoid tissues and peripheral blood in HBV infected mice were investigated by FACS analysis. T cell lineages (including CD4⁺/CD8⁺ T cells, naïve T cells, memory T cells and regulatory T cells), B cells, dendritic cells (including mDCs, pDCs), granulocytes and monocytes/macrophages were analyzed. RAAS 105 was administered in two different time schedules for therapeutic and prophylactic treatment.

Therapeutic treatment revealed some interesting findings. The animals treated with RAAS 105 exhibited alterations in multiple immune cells and various lineages compared with that in the vehicle group, including reduction of lymphocytes and increase of granulocytes and monocytes. Prophylactic treatment led to less dramatic alterations in the immune cells.

Final Report

Efficacy of Eight RAAS Test Articles on Adjuvant-Induced Arthritis (AIA) in Lewis Rats Executive Summary

| | |
|---|---|
| AIA | Adjuvant-induced arthritis |
| Dex | Dexamethasone |
| i.p. | intraperitoneal |
| HPMC | (Hydroxypropyl) methyl cellulose |
| p.o. | Per oral |
| b.i.d. | Twice a day |
| q.d. | Once a day |
| N/A | Not available |

This study has evaluated the efficacy of eight RAAS test articles in the treatment of Adjuvant-Induced Arthritis (AIA) in Lewis rats. Male Lewis rats were immunized with Mycobacterium tuberculosis H37Ra to elicit AIA. On day 11 after immunization, when all the animals developed arthritis, the rats were administered with saline, Dexamethasone (Dex, positive control), and eight RAAS test articles for various durations, according to the sponsor's requests. The detailed treatment regimen is described below.

The data from this study showed that after the onset of the disease, the treatment with all eight RAAS products did not significantly affect the disease progression. After treatments, all the groups maintain 100% incidence rate. However, the group of animals treated with Dex had very mild disease, demonstrating dramatic inhibitory effects on the arthritic response. On the contrary, all the groups of rats treated with different RAAS products showed severe arthritis. The arthritic scores are similar among all the groups treated with RAAS products compared to that of vehicle group. Nevertheless, the measurement of paw swelling indicated that the paw volumes of the animals treated with AFCC KH and AFOD 101 decreased but the differences were not significant statistically at the most of the times compared to the vehicle group.

A. List of Abbreviations

B. Materials and Methods

a. Experimental Groups

The original study was planned to do the treatment for 10 days after disease onset. Table 1 was the group setting and dosing regimen.

TABLE 1
Grouping and Dosing Regimen for Day 11 to 20.

| | | | | Conc. | Dose vol. | |
|---|---|---|---|---|---|---|
| Group | Test Article | N | Route | mg/ml | ml/rat | Frequency |
| 1 | Normal | 5 | N/A | N/A | N/A | N/A |
| 2 | Vehicle (Saline) | 8 | i.p. | N/A | 3 | q.d. |
| 3 | Dex ^{a} | 8 | p.o. | 0.02 | 5 ml/kg | q.d. |
| 4 | AFCC KH | 8 | i.p. | 18% | 3 | q.d. |
| 5 | AFOD KH | 8 | i.p. | 20% | 3 | q.d. |
| 6 | AFOD 101 | 8 | i.p. | 20% | 3 | q.d. |
| 7 | AFOD 102 | 8 | i.p. | 5% | 3 | q.d. |
| 8 | AFOD 103 | 8 | i.p. | 5% | 3 | q.d. |
| 9 | AFOD 107 | 8 | i.p. | 1% | 3 | q.d. |
| 10 | AFOD 108 | 8 | i.p. | 2.5% | 3 | q.d. |
| 11 | AFOD 1 | 8 | i.p. | 5% | 3 | q.d. |

^{a} 0.5% HPMC/0.02% Tween 80 made with MilliQ water as vehicle

After the completion of 10-day treatment, the sponsor requested to continue the treatment for 15 more days and to increase dosing volumes (from 3 ml/rat/day q.d., to 2.5 ml/rat/day b.i.d.) as indicated in Table 2.

TABLE 2
Grouping and Dosing Regimen for Day 21 to 35

| | | | | Conc. | Dose vol. | |
|---|---|---|---|---|---|---|
| Group | Test Article | N | Route | mg/ml | ml/rat | Frequency |
| 1 | Normal | 5 | N/A | N/A | N/A | N/A |
| 2 | Vehicle (Saline) | 8 | i.p. | N/A | 2.5 | b.i.d. |
| 3 | Dex ^{a} | 8 | p.o. | 0.02 | 5 ml/kg | q.d. |
| 4 | AFCC KH | 8 | i.p. | 18% | 2.5 | b.i.d. |
| 5 | AFOD KH | 8 | i.p. | 20% | 2.5 | b.i.d. |
| 6 | AFOD 101 | 8 | i.p. | 20% | 2.5 | b.i.d. |
| 7 | AFOD 102 | 8 | i.p. | 5% | 2.5 | b.i.d. |
| 8 | AFOD 103 | 8 | i.p. | 5% | 2.5 | b.i.d. |
| 9 | AFOD 107 | 8 | i.p. | 1-2% | 2.5 | b.i.d. |
| 10 | AFOD 108 | 8 | i.p. | 2.5% | 2.5 | b.i.d. |
| 11 | AFOD 1 | 8 | i.p. | 5% | 2.5 | b.i.d. |

^{a} 0.5% HPMC/0.02% Tween 80 made with MilliQ water as vehicle

After the completion of 25-day treatment, the sponsor requested additional 7 days treatment for five groups—Saline, Dex, AFCC KH, AFOD 101 and AFOD 102, as listed in Table 3. Please note that there was a two-day gap (Day 36 and 37) without treatment, before starting this 7-day period of treatment.

TABLE 3
Grouping and Dosing Regimen for Day 38 to Day 45:

| | | | | Conc. | Dose vol. | |
|---|---|---|---|---|---|---|
| Group | Test Article | N | Route | mg/ml | ml/rat | Frequency |
| 1 | Normal | 5 | N/A | N/A | N/A | N/A |
| 2 | Vehicle (Saline) | 8 | i.p. | N/A | 2.5 | b.i.d. |
| 3 | Dex ^{a} | 8 | p.o. | 0.02 | 5 ml/kg | q.d. |
| 4 | AFCC KH | 8 | i.p. | 18% | 2.5 | b.i.d. |
| 6 | AFOD 101 | 8 | i.p. | 20% | 2.5 | b.i.d. |
| 7 | AFOD 102 | 8 | i.p. | 28% | 2.5 | b.i.d. |

^{a} 0.5% HPMC/0.02% Tween 80 made with MilliQ water as vehicle

b. Material

i. Reagents

Mycobacterium tuberculosis H37Ra: Difico (Detroit, Mich., USA), Cat: 231141

Paraffin oil: China National Medicine Corporation Ltd, Cat: 30139828

Hydroxypropyl Methyl Cellulose: Sigma, Cat: C5135

Tween 80: Sigma, Sigma-Aldrich. (St. Louis, Mo., USA), Cat: P-4780

Saline: Jiangsu Kang Bao Pharmaceutical Co., Ltd. Cat: H32026295

Dexamethasone (Dex): Xinyi Pharmaceutical Co., Ltd, H31020793

ii. Dose Formulation and Storage

All test articles were provided by the sponsor and storage at 4° C. before use.

iii. Equipment

Plethysmometer, Italy UGO BASJLE, Biological Research Apparatus 21025

iv. Animals and Testing Facility
Species: Rat
Strain: Lewis
Vendor: Beijing Vital Rivers Laboratories
Sex: Male
Body Weight when study started 180-200 g
Test Facility: WuXi AppTec Vivarium
Food: Free access to food (irradiated, Shanghai SLAC Laboratory Animal Co. Ltd., China)
Water: Free access to water (municipal tap water filtered by Mol Ultrapure Water System)
Total number of animals 85
Animal housing: 4 Rats/cage by treatment group
Identification Each rat was identified by ear tag and cage card
Adaptation: At least 7 days
Room: SPF Room
Room temperature: 20-26° C.
Room relative humidity: 40-70%
Light cycle: Fluorescent light for 12-hour light (6:00-18:00) and 12-hour dark (18:00-6:00)
Allocation to treatment groups: Randomization into 11 groups to achieve similar mean body weight, minimizing bias (See Table 1).

NOTE: All of the experimental procedures carried out within this study were approved by IACUC at WuXi AppTec.

v. Test Article Preparation

Dex: Dex was dissolved with 0.5% HPMC/0.02% Tween 80 into a final concentration of 0.02 mg/ml. The dosing volume is 5 ml/kg. Sonicate the suspension in an ice water bath for 10 minutes. Four 12 ml aliquots were stored in 4° C. refrigerator before use.

RAAS test article: Right before each dosing, a 50 ml of aliquot of each test article was prepared and warmed to room temperature.

vi. Immunization

Adjuvant Preparation
Weigh 100 mg of heat-killed Mycobacterium tuberculosis, ground suspended in Paraffin oil to final concentration of 10 mg/ml.
Sonicate the suspension in an ice water bath for 15 minutes.

Immunization Procedure
Shake the suspension of heat-killed/VI tuberculosis in Paraffin oil (to ensure even distribution of bacterial particles), then draw suspension into a 1 ml glass syringe attached to a 20-G needle. Replace the needle on the glass syringe with a 25-G needle. Re-suspend material in glass syringe by rolling between hands.
Anesthetize the rats with isoflurane, then inject 0.1 ml M. tuberculosis suspension subcutaneously in the left hind foot pad.
For the normal group (n=5), mineral oil was injected subcutaneously in the left hind foot pad.
80 rats were randomly allocated to 10 groups (Table 1). The day of the injection was considered as day 0.

vii. Treatment
The treatment started at Day 11 as instructed by the sponsor. The incidence rates were 100%. The original planned treatment was 10 days (Day 11 to 20), with the dosage and dosing routes indicated in Table 1.
Per sponsor's request, all eight test articles were continued treated for additional 15 days (Day 21 to 35), with increased dosage. The detailed dosage and regimen was listed in Table 2.
The sponsor requested another additional 7 days (Day 38 to 45) of treatment for Saline, Dex, AFCC KH, AFOD 101 and AFOD 102 groups (Table 3). There was a two days gap (Day 36 and 37) before this segment.

viii. Endpoints
Body weight: Body weight of each animal was recorded every two days.
Paw swelling: The volume of right hind paw was pre-measured before immunization, and the right hind paw was measured once every two days, from Day 7 with plethysmometer.
Arthritic score: Start from Day 7 to 45, evaluate disease development by macroscopic inspection every two days. Assess walking ability, and screen for skin redness and swelling at the site of ankle and wrist joints and small interphalangeal joints. The left hind foot (the injected paw) will be excluded, the highest score is 12. See the criteria in table 4.

TABLE 4
Scoring system for evaluate arthritis severity

| Score | Clinical signs |
|---|---|
| 0 | No erythema or swelling |
| 1 | Slight erythema and swelling in one of the toes or fingers |
| 2 | Erythema and swelling in more than one toe or finger or mild |
| | swelling extending from the ankle to the mid-foot |
| 3 | Eryghema and severe swelling in the ankle or wrist |
| 4 | Complete erythema and swelling in toe or fingers and ankle or |
| | wrist, and inability to bend the ankle or wrist |

C. Data Analysis

Data were presented as mean±SEM. The body weight and paw volume were analyzed with two-way repeated ANOVA and the arthritis scores with Kruskal-Wallis test, by Graph Pad Prism 5. The statistical significance was noted when p<0.05.

D. Study Summary

a. Study Initiation Date and Completion Date

The study was initiated on Aug. 10, 2012, and ended on Sep. 24, 2012

b. Study Purpose

The goal of this project is to examine eight RAAS products in an autoimmune arthritis model, adjuvant induced arthritis (AIA) in rats. The study is to determine whether the products have therapeutic effects on AIA.

c. Study Results

The results of eight test articles are presented in two sections, according to their treatment durations: 1) 35 days treatment for AFOD KH, AFOD 103, AFOD 107, AFOD 108 and AFOD 1; 2) 45 days treatment for AFCC KH, AFOD 101 and AFOD 102.

i. Body Weight

Except Dex group, there was no significant difference for the body weight of all the treatment groups, when compared with saline group, in both 35 days and 45 days treatment sections (FIGS. 1 and 2). The reduction of body weight in Dex group was due to the side effect of Dex treatment.

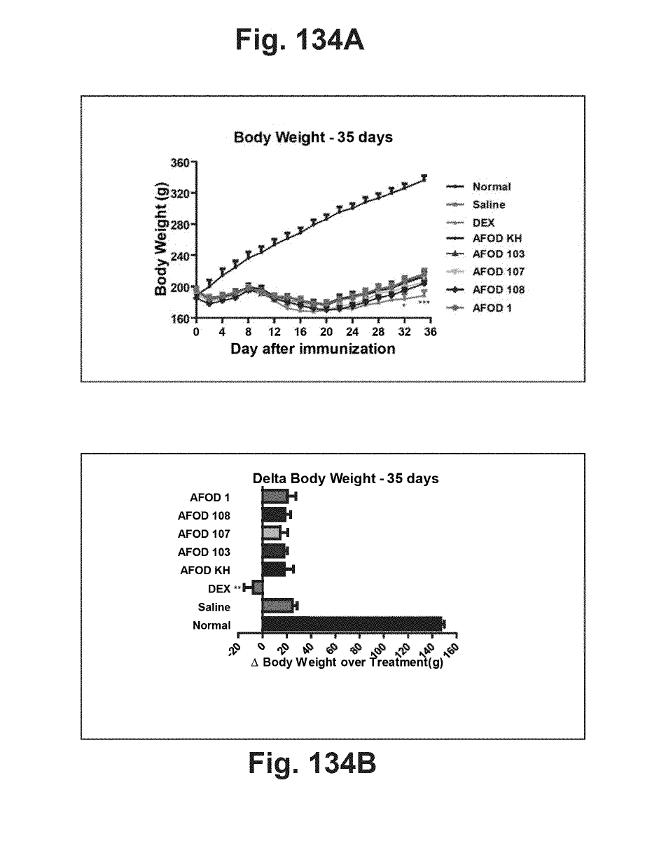

FIG. 134. Effects of AFOD KH, AFOD 103, AFOD 107, AFOD 108 and AFOD 1 on body weight (A) and body weight change (B) in AIA model till Day 35 (*p<0.05, **p<0.01, ***p<0.001, treatment groups v.s. saline group, two-way repeated or one-way ANOVA).

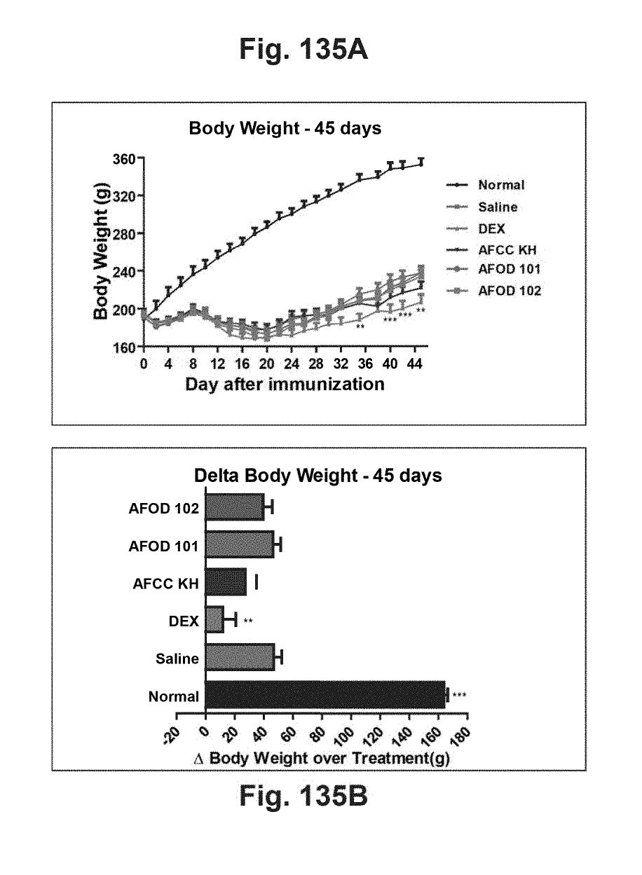

FIG. 135. Effects of AFCC KH, AFOD 101 and AFOD 102 on body weight (A) and body weight change (B) in AIA model till Day 45 (**p<0.01, ***p<0.001, treatment groups v.s. saline group, two-way repeated or one-way ANOVA).

ii. Paw Volume

The measurement of the paw volume indicated that the paw swelling was slightly reduced in the groups of animal treated with AFCC KH and AFOD 101. Statistical analysis showed that at the most of the times, the reduction was not significant statistically. However, the animals treated with AFCC KH showed significantly reduced paw volume on Day 22 and 35, compared to that of saline group (FIG. 4A). The animals treated with AFOD 101 showed significantly reduced paw swelling on day 22 (FIG. 4A). All other groups treated with the other six RAAS products didn't show any significant reduction in the paw swelling (FIGS. 3B & 4B).

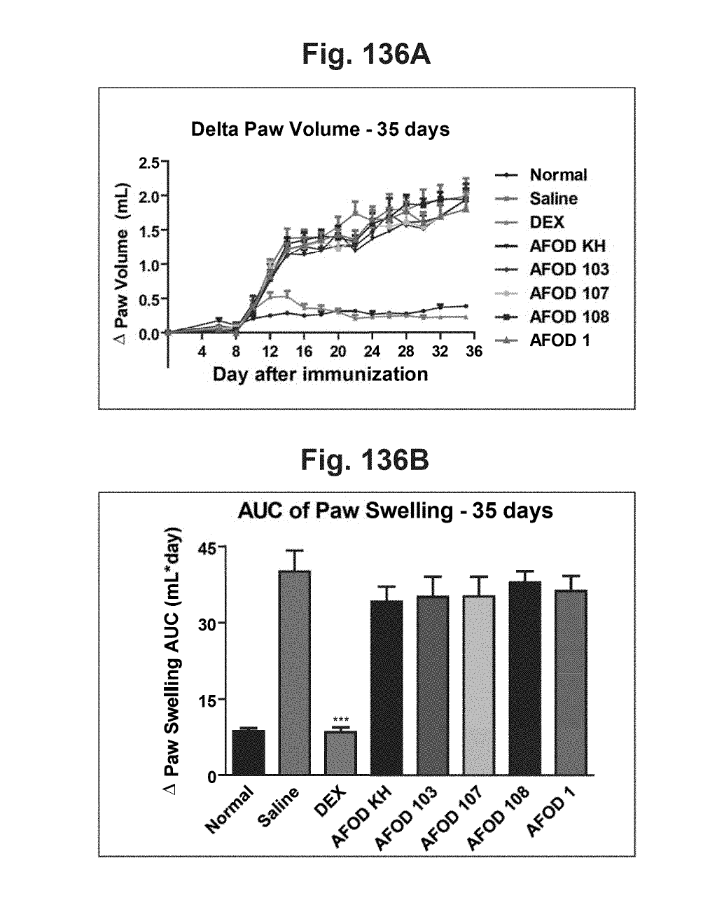

FIG. 136. Effects of AFOD KH, AFOD 103, AFOD 107, AFOD 108 and AFOD 1 on delta paw (right hind paw) volume (A) in AIA model till Day 35. AUC of delta paw volume curves were also presented (B). The delta paw volume of Dex group was significantly lower than saline group, from day 14 (***p<0.001, v.s. saline group, two-way repeated or one-way ANOVA).

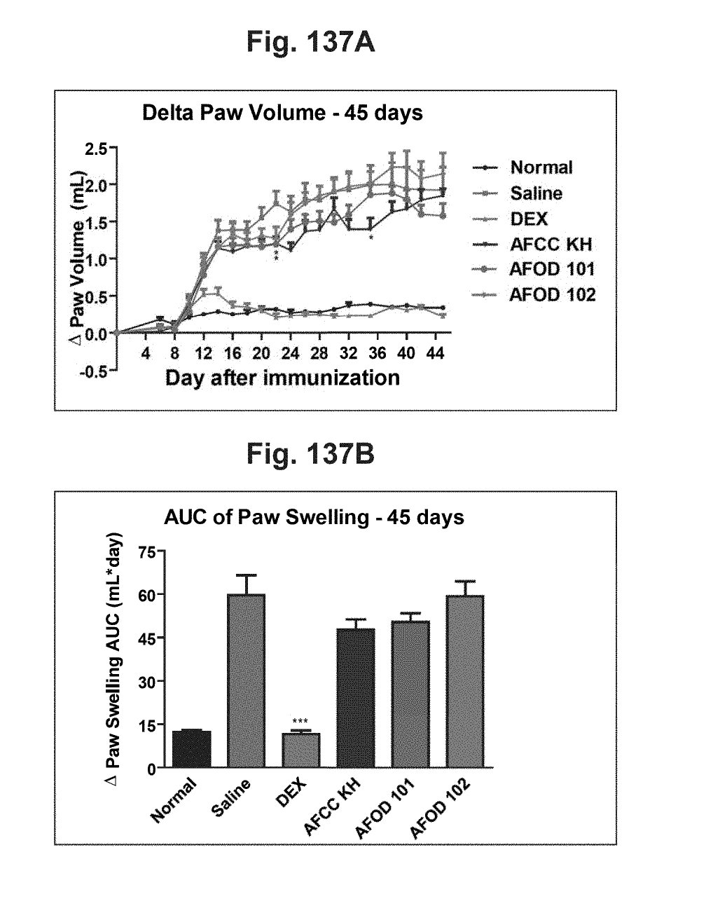

FIG. 137. Effects of AFCC KH, AFOD 101 and AFOD 102 on delta paw (right hind paw) volume (A) in AIA model till Day 45. AUC of delta paw volume curves were also presented (B). The delta paw volume of Dex group was significantly lower than saline group, from day 14 (***p<0.001, v.s. saline group, two-way repeated or one-way ANOVA).

iii. Arthritic Score

The arthritic scores in all the groups treated with the eight test articles were similar to that of vehicle group (FIGS. 5 & 6). Dex treatment significantly inhibited the disease development (FIGS. 5 & 6).

FIG. 138. Effects of AFOD KH, AFOD 103, AFOD 107, AFOD 108 and AFOD 1 on arthritic score in AIA model till day 35. The arthritic score of Dex group was significantly lower than saline group, from day 14 (p<0.01 for day 14, p<0.001 for day 16 to 35, Kruskal-Wallis test).

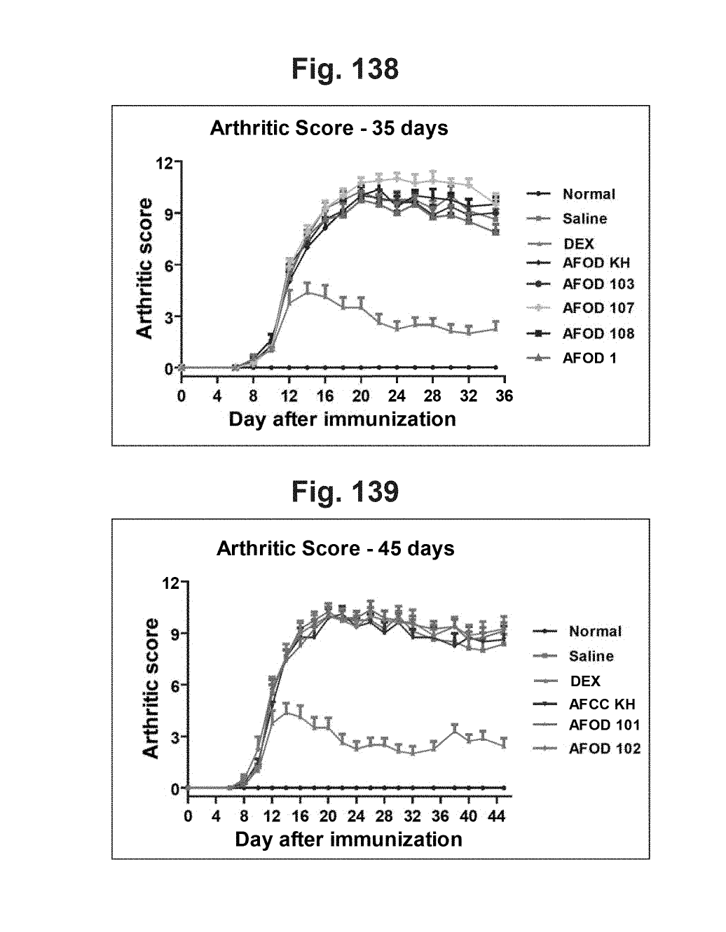

FIG. 139. Effects of AFCC KH, AFOD 101 and AFOD 102 on arthritic score in AIA model till Day 45. The arthritic score of Dex group was significantly lower than saline group, from day 14 (p<0.01 for day 14, p<0.001 for day 16 to 45, Kruskal-Wallis test).

iv. Incidence Rate

All the animals immunized with adjuvant developed arthritis at day 11 after immunization, when the treatment started, per sponsor's request. The incidence rates of all the groups remained 100% throughout the study period (FIGS. 7 & 8).

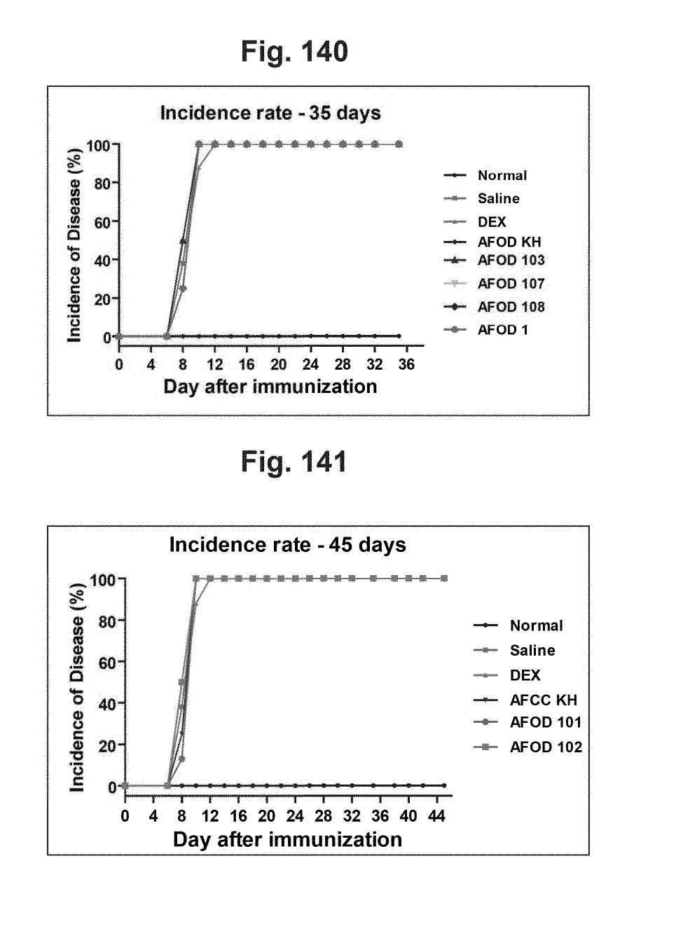

FIG. 140. Effects of AFOD KH, AFOD 103, AFOD 107, AFOD 108 and AFOD 1 on incidence rate in AIA model till day 35. The incidence rate reached 100%, 11 days after immunization. There was no chance of incidence rate afterward, for all the treatment

FIG. 141. Effects of AFCC KH, AFOD 101 and AFOD 102 on incidence rate in AIA model till day 45. The incidence rate reached 100%, 11 days after immunization. There was no change of incidence rate afterward. for all the treatment groups.

E. Conclusion
The treatment of eight test articles did not significantly affect the body weight changes compared to the saline group. The body weight of Dex group was lower than the other groups after treatment from Day 11.
Overall, the treatment of eight test articles did not inhibit paw swelling significantly after 25-day or 32-day treatments. However, the group of animals treated with AFCC KH and AFOD 101 showed reduced paw swelling. Statistical analysis showed significant difference for AFCC KH and AFOD 101, but only on Day 22, 35 and Day 22 respectively, by comparing to vehicle group.
Based on the arthritic scores, all the treatments did not show significant impacts on the disease progression. Dex treatment significantly inhibited the disease development.
The incidence rate reached 100% after day 11, before the treatment started, demonstrating successful setup of the model. During the treatment from day 11 to day 45, the incidence rates in all the groups remained 100%.

F. Reference

Debra M Meyer, Michael I Jesson, Xiong Li. Anti-inflammatory activity and neutrophil reductions mediated by the JAK1/JAK3 inhibitor CP-690,550, in rat adjuvant-induced arthritis 2010.7.1

Study Title: Efficacy Study of AFOD RAAS 1 (APOA1) on Atherosclerosis Model in ApoE Mice

1. Abbreviations and Definitions

kg kilogram

g gram

Mg milligram

ng Nano gram

ml Milliliter

microliter

h hours

min minutes

Cpd Compound

BW Body Weight

BG Blood Glucose

FBG Fasting Blood Glucose

DOB Date of Birth

TC Total Cholesterol

TG Triglyceride

LDL Low Density Lipoprotein

HDL High Density Lipoprotein

FBW Fasting Blood Glucose

SD Standard Deviation

SE Standard error

i.p Intraperitoneal injection

PFA paraformaldehyde

2. Introduction

The study described in this report evaluated in vivo efficacy of RAAS antibody APOA I on atherosclerotic model in ApoE knockout mice.

3. Purpose

To evaluate the efficacy effect of RAAS antibody APO AI on plasma lipid profile, plaque lesion of inner aorta and related parameters in atherosclerotic model.

4. Materials
4.1. Test article: RAAS Apo A I; Atorvastatin (reference compound)
4.2. Animal: ApoE knock out (ko) mouse
   Sex: male
   Strain: C57BL/6
   Vender: Beijing Vitol River
   Age: 8 weeks (arrived on 23 Dec. 2011)
   Number: 60
4.3. Lipid profile test: Shanghai DaAn Medical Laboratory, Roche Modular automatic biochemistry analyzer
4.4. Heparin Sodium Salt: TCI, H0393
4.5. Capillary: 80 mm, 0.9-1.1 mm
4.6. Ophthalmic Tweezers and scissors: 66 vision-Tech Co., LTD, Suzhou, China. Cat#53324A, 54264TM
4.7. High Fat diet: TestDiet, Cat#58v8(35% kcal fat 1% chol)
4.8. Glycerol Jelly Mounting Medium: Beyotime, Cat# C0187.
4.9. Glucose test strips: ACCU-CHEK Performa: ROCHE (Lot#470396)
4.10. Image analyse: Aperio ScanScope system; Image-Proplus 6.0 software; Aperio image scope version 11.0.2.725 software.
4.11. Aorta staining: Oil Red O (Alfa Aesar) Isopropanol (Lab partner)

5. Experiment Method

5.1. Grouping Mice:

10 ApoE ko mice were fed with regular chow diet and used as negative control group. 50 ApoE ko mice were fed with high fat diet (35% kcal fat, 1% cholesterol) for 8 weeks, and then the plasma samples were collected for lipid profile measurement before the treatment. 50 ApoE ko mice were assigned into 5 groups based on the fasting overnight plasma TC and HDL level. The group information is shown in the table below.

TABLE 1
Information of groups

| | ApoE ko | | | Conc. Of | |
|---|---|---|---|---|---|
| Group | mice | Diet | Solution | CPD | Formulation |
| Negative Control | n = 10 | Normal diet | | | |
| Vehicles (saline) | n = 10 | High fat diet | 0.9% | | |
| | | | NaCL | | |
| ApoA1 High Dose: 0.1 ml i.p q.o d | n = 10 | High fat diet | | 5% Protein | |
| ApoA1 Mid Dose: 0.075 ml i.p q.o d | n = 10 | High fat diet | | 5% Protein | |
| ApoA1 Low Dose: 0.0.05 m i.p q.o | n = 10 | High fat diet | | 5% Protein | |
| Positive Control (Atorvastatin) | n = 10 | High fat diet | 0.5% CMC | 2 mg/mL | 20 mg + 10 ml |
| 20 mg/kg (increased to 40 mg/kg) | | | | | 0.5% CMC |

5.2. Study Timeline:
23 Dec. 2011: 60 ApoE mice arrived at chempartner and were housed in the animal facility in the building #3 for the acclimation.
6 Jan. 2012: Measured the body weight for each mouse. 50 mice were fed with high fat diet and 10 mice were fed with normal chow diet.
2 Mar. 2012: All mice were fasted over night and plasma samples (about 300 ul whole blood) were collected for lipid profile measurement before treatment with RAAS antibody.
19 Mar. 2012 to 6 Apr. 2012: Group the mice based on the TC and HDL level and start the treatment with 3 doses of antibody APOA1 by i.p daily on the weekday (The first dose was administered by iv injection via the tail vein. The reference compound atorvastatin was administered by oral dosing every day.
7 Apr. 2012 to 12 Apr. 2012: Stop dosing for 5 days. After 15 doses treatment with the antibody, several mice died in the treatment groups. The client asked for stopping treatment for a while.
13 Apr. 2012-6 Jul. 2012: The treatment with antibody APOA1 was changed to i.p injection every two days (Monday, Wednesday, and Friday) per client's instruction.
14 May 2012: All mice were fasted over night and plasma sample for each mouse (about 300 ul whole blood) was collected for lipid profile measurement after 8 weeks treatment.
9 Jul. 2012: All mice were fasted over night and plasma sample for each mouse (about 300 ul whole blood) was collected for lipid profile measurement after 16 weeks treatment. Blood glucose was also measured for each mouse.
9 Jul. 2012: The study was terminated after 16 weeks treatment. Measure BW, sacrificed each mouse, dissected the aorta, heart, liver and kidney and fixed them in 4% PFA.

5.3. Route of Compound Administration:

Antibody products were administrated by intraperitoneal injection every two days (Monday, Wednesday, and Friday). and the positive compound was administered by p.o every day.

5.4. Body Weight and Blood Glucose Measurement:

The body weight was weighed weekly during the period of treatment. The fasting overnight blood glucose was measured at the end of study by Roche glucometer.

5.5 24 h Food Intake Measurement:

24 hours food intake for each cage was measured weekly

5.6. Plasma Lipid Profile Measurement:

About 300 ul of blood sample was collected from the orbital vein for each mouse and centrifuged at 7000 rpm for 5 min at 4E and the plasma lipid profile was measured by Roche Modular automatic biochemistry analyzer in DaAn Medical Laboratory

5.7. Study Taken Down:

After RAAS antibody products treatment for 16 weeks, all mice were sacrificed. Measured body weight and collected blood sample for each mouse. Weighed liver weight and saved a tiny piece of liver into 4% paraformaldehyde (PFA) fixation solution for further analysis. At same time, take the photos with heart, lung, aortas and two kidneys.

5.8. Oil Red Staining Procedure:
1. Sacrificed the mice and dissected the heart, aorta, and arteries under dissecting microscope.
2. Briefly wash with PBS and fixed in 4% paraformaldehyde (PFA) overnight at 4□.
3. Rinse with 60% isopropanol
4. Stain with freshly prepared Oil Red O working solution 10 min.
   1). Oil red O stock stain: 0.5% powder in isopropanol
   2). Working solution: dilute with distilled water (3:2) and filter with membrane (0.22 um)
5. Rinse with 60% isopropanol 10 second.
6. Dispel the adherent bit fat outside of the aorta under the dissecting microscope.
7. Cut the vascular wall gently and keep the integrated arteries using the micro scissors.
8. Unfold the vascular inner wall with the cover slides and fix it by water sealing tablet.

5.9. Image Scanning and Analysis:

Scanning the glasses slides with the Aperio ScanScope system and analyze with the image proplus software to measure the area of atherosclerotic plaque lesion. The results were expressed as the percentage of the total aortic surface area covered by lesions. The operation procedure of software was briefly described as follow: Converted the sys version photos into JPG version, then calibrated it and subsequently selected the red regions and then calculate the total area automatically by image proplus software.

5.10. Clinic Observation:

The information of dead animals was shown in the table as below.

6. Data Analysis

The results were expressed as the Mean±SEM and statistically evaluated by student's t-test. Differences were considered statistically significant if the P value was <0.05 or <0.01.

7. Results

7.1. Effect of APOA 1 on Body Weight

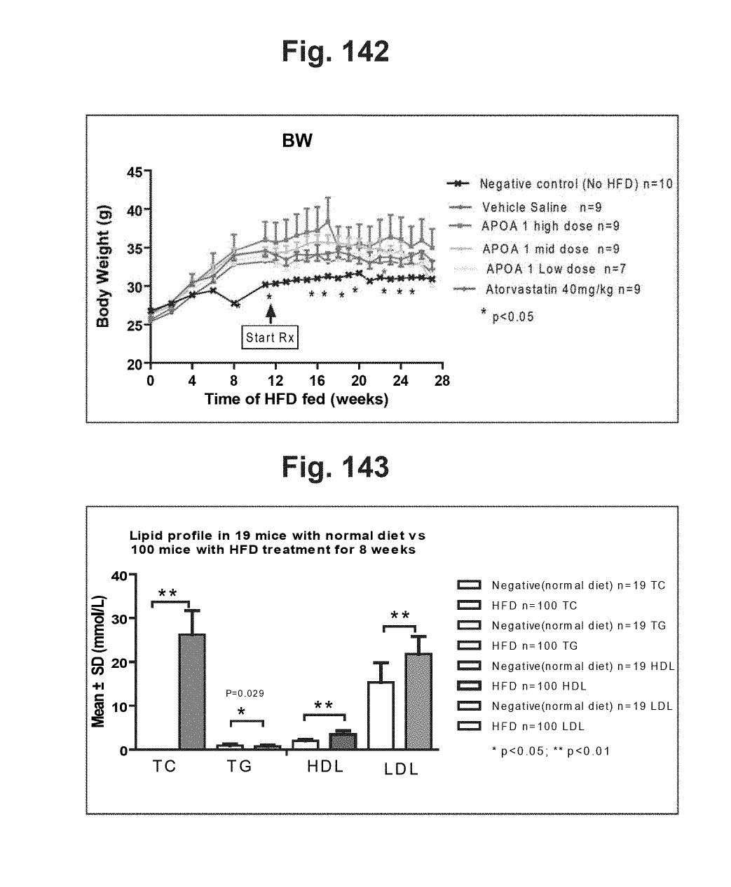

FIG. 142. Body weight

The body weight in Apo E knockout mice fed with HFD significantly increased after 6 weeks treatment compared with the mice in negative control group that were fed with normal diet. There is no significant difference between the treatment groups and vehicle group.

7.2. Effect of HFD on Lipid Profile in ApoE Ko Mice

FIG. 143. Plasma lipid profile of ApoE mice fed with normal diet and high fat diet.

The lipid profile was measured in Apo E ko mice fed with high fat diet for 8 weeks. As shown above, plasma TC, TG, LDL as well as HDL in Apo E ko mice fed with high fat/high cholesterol for 8 weeks were significantly increased compared to Apo E KO mice fed with normal chow diet.

7.3. Effect of RAAS Antibody on Plasma Total Cholesterol (TC)

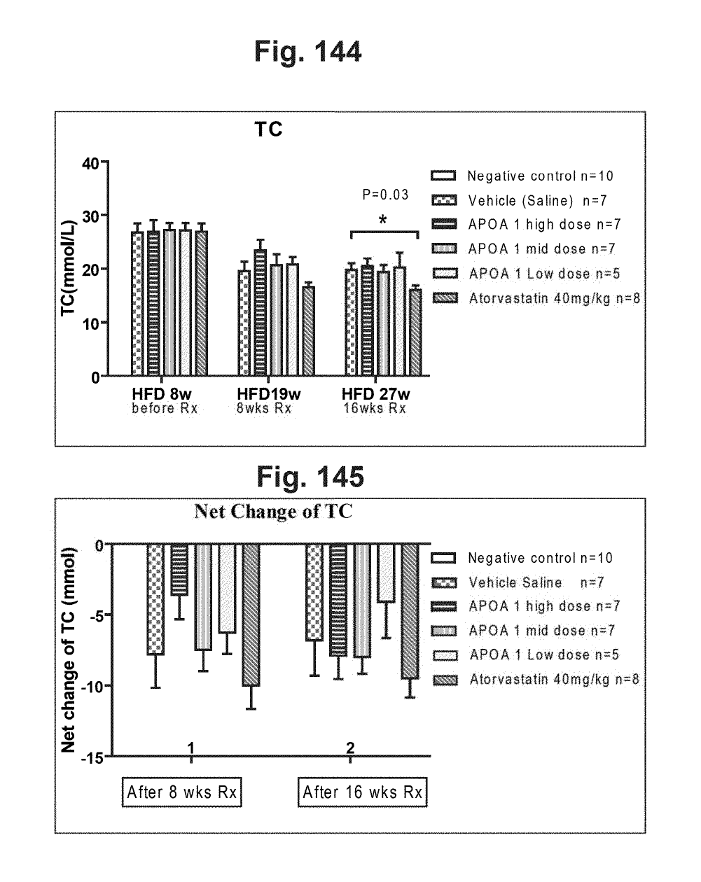

FIG. 144, Plasma TC

FIG. 145. Net change of plasma TC

As shown in the figure above, positive control atorvastatin can significantly lower total cholesterol level after 16 week treatment in ApoE ko mice but not reduce the TC net change.

7.4. The Effect of RAAS Antibody on Plasma Triglyceride (TG)

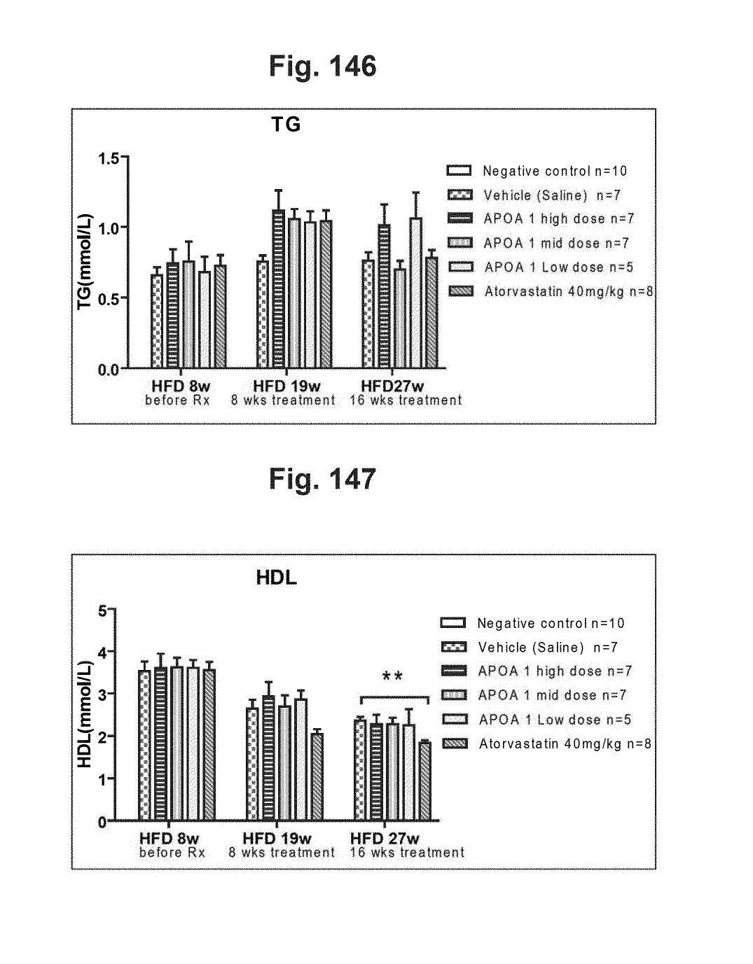

FIG. 146. Plasma TG

As shown in figure above, positive control atorvastatin and RAAS antibody had no effect on plasma TG level in Apo E ko mice fed with HFD after 16 weeks treatment.

7.5. The Effect of RAAS Antibody on High Density Lipoprotein (HDL)

FIG. 147. Plasma HDL

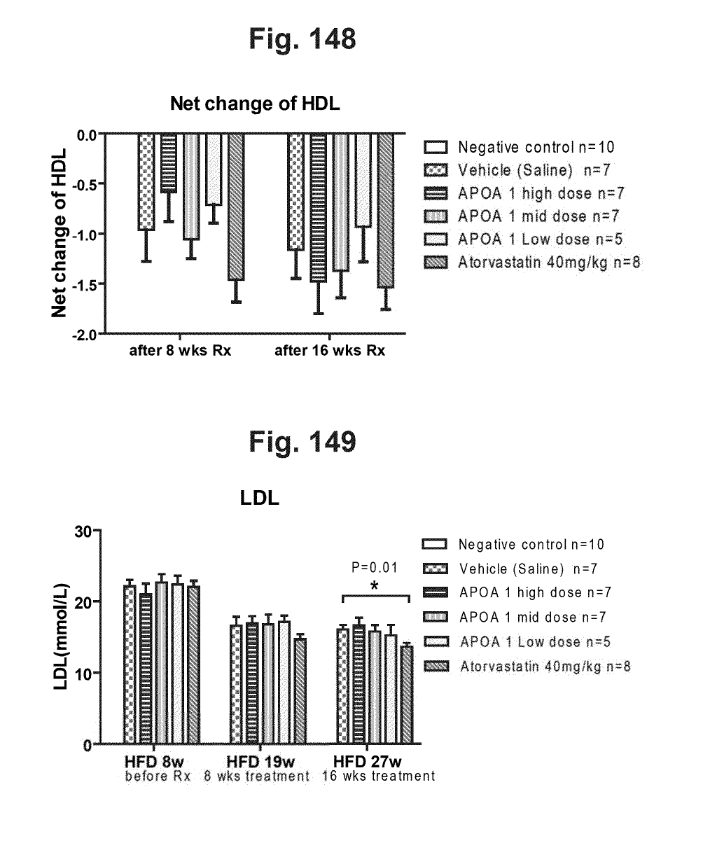

FIG. 148. Plasma HDL net change

As shown in figure above, positive control atorvastatin can significantly lower high density lipoprotein in Apo E ko mice fed with HFD after 16 week treatment and RAAS antibody had a mild trend to decrease the HDL level in ApoE ko mice after 16 weeks treatment.

7.6. The Effect of RAAS Antibody on Low Density Lipoprotein (LDL)

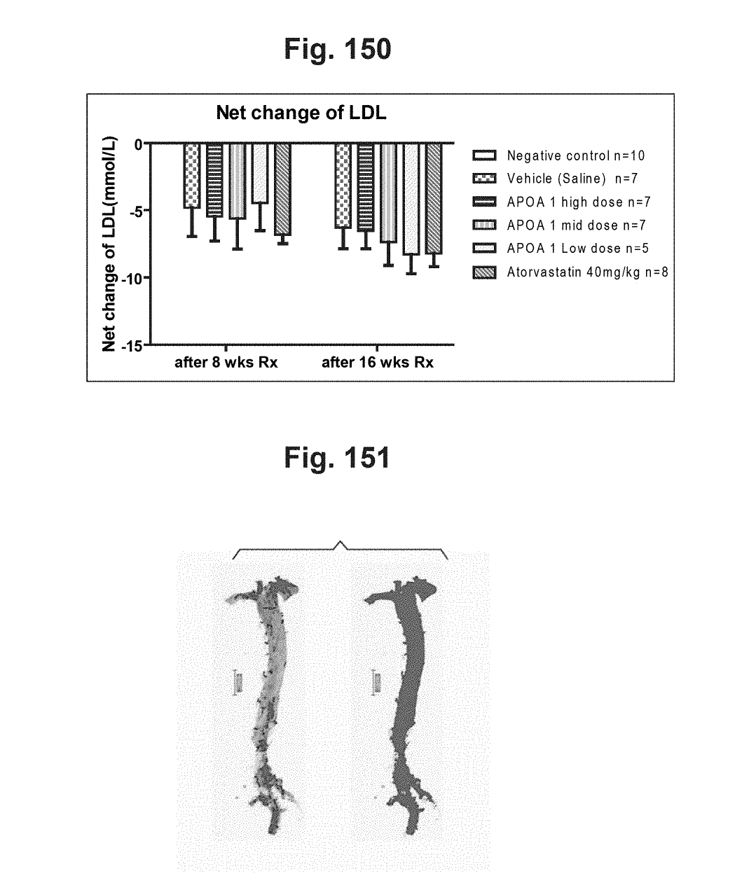

FIG. 149. Plasma LDL level

FIG. 150. Plasma HDL net change

As shown in figure above, positive control atorvastatin can significantly decrease low density lipoprotein in Apo E ko mice fed with HFD after 16 week treatment and there is no significant difference in net change of LDL.

7.7. The Effect of RAAS Antibody on Atherosclerosis Plaque Lesion

FIG. 151. Illustrated by negative control group animal #10

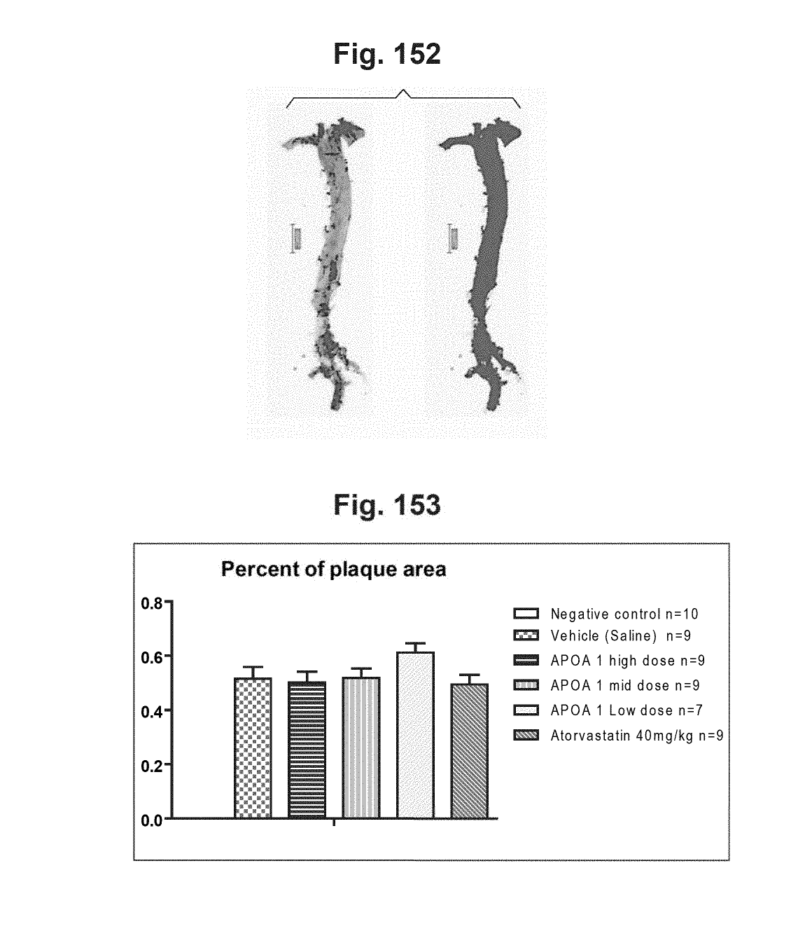

FIG. 152. Illustrated by negative control group animal #10 As shown in the above diagram, we calculated all the plaque area stained by oil red and divided by total inner vascular area

Area percent (%)=Sum area of atherosclerotic plaque (mm²)/whole area of vascular inner wall (mm²)

FIG. 153. Percent of plaque area in total inner vascular area

No significant difference between the vehicle and treatment groups in plaque area and percentage of plaque area although Atorvastatin showed a mild trend to decrease percentage of plaque area after 16 weeks oral administration.

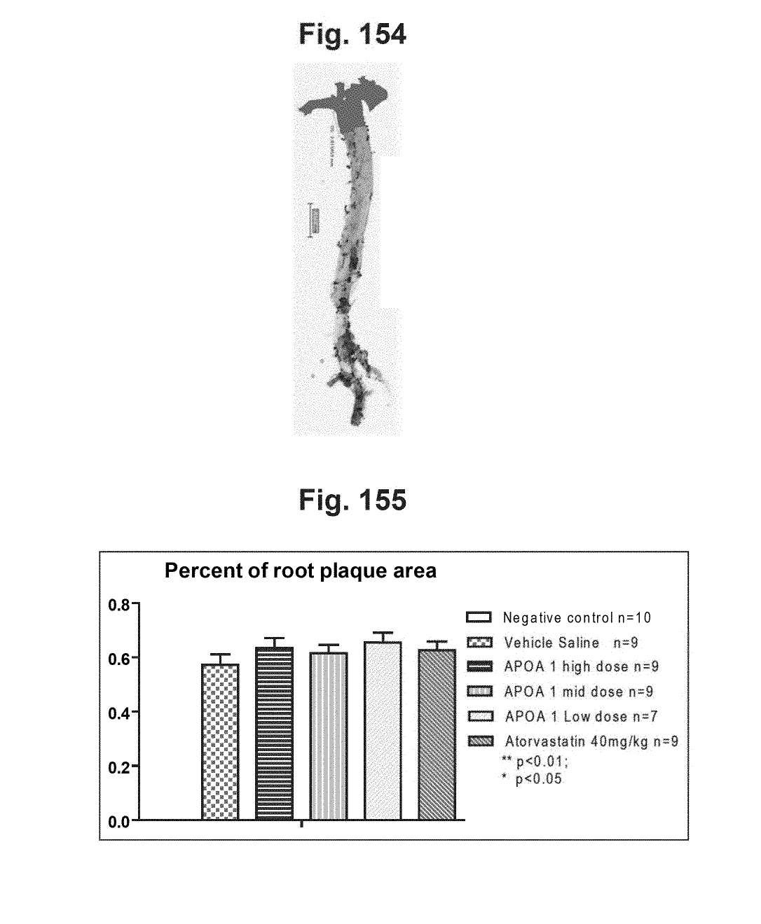

FIG. 154. Illustrated Analysis of arterial arch area

The total area of aorta from the aortic root to the thoracic aorta was measured (bracketed area).

As shown in the left panel, because the total lumen area in arterial arch is very difficult to identify in en face vessel, we measured the total area at the length of about 2 mm from aortic root down to the thoracic artery (bracketed area).

FIG. 155. Percent of plaque area in arterial arch area

The plaque lesion was more severe in mice fed with HFD than mice in the normal diet (negative) group. No significant difference between the vehicle and treatment groups in plaque area and percentage of plaque area.

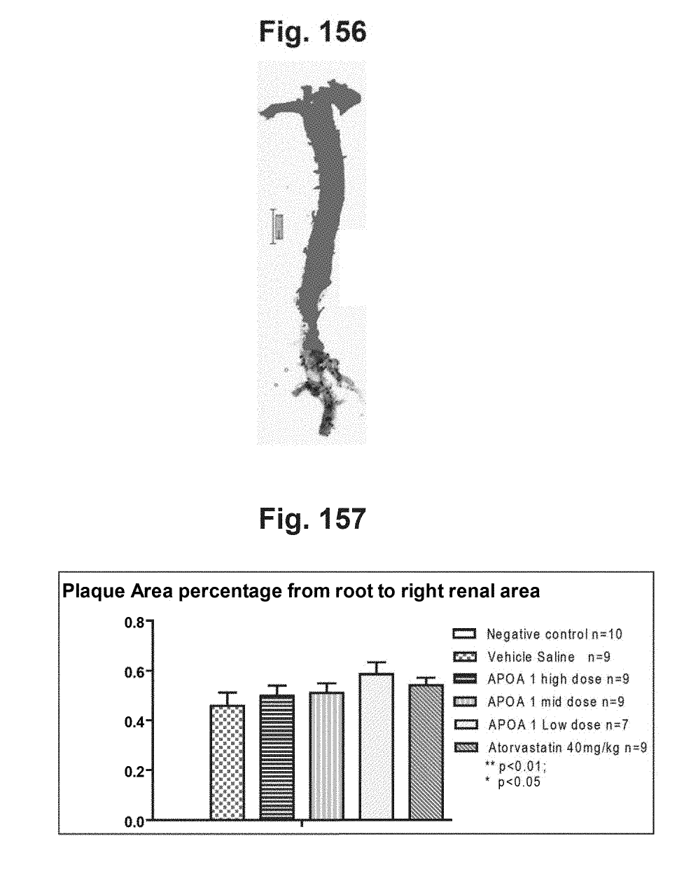

FIG. 156. Illustrated Analysis of from root to right renal artery

As shown in the left panel, the total area from the aortic root to the right renal artery were measured (bracketed area)

FIG. 157. Percent of plaque area from root to right renal artery

There is no significant difference between vehicle and treatment groups in plaque area and percentage of plaque area.

7.8. The Effect of RAAS Antibody on Liver Weight

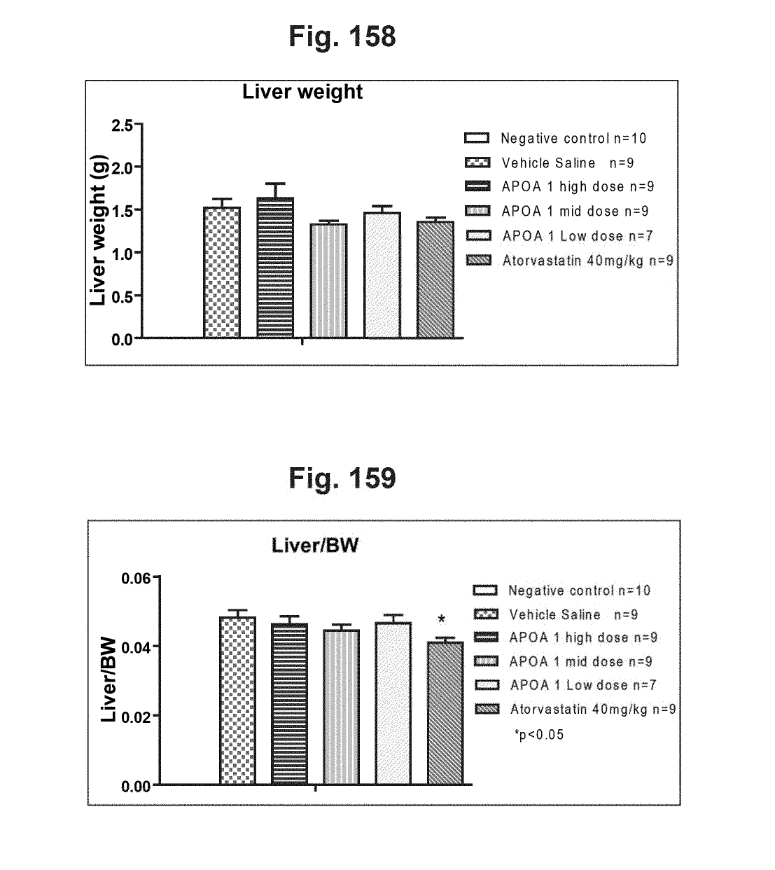

FIG. 158. Diagram of liver weight

FIG. 159. Diagram of liver index

Atorvastatin at 20 mg/kg reduced the ratio of liver/body weight significantly after 16 weeks treatment, which is consistent with the 8 weeks treatment result in study 2.

7.9 Comparison of Percentage of Plaque Area in Study 1, 2, 3

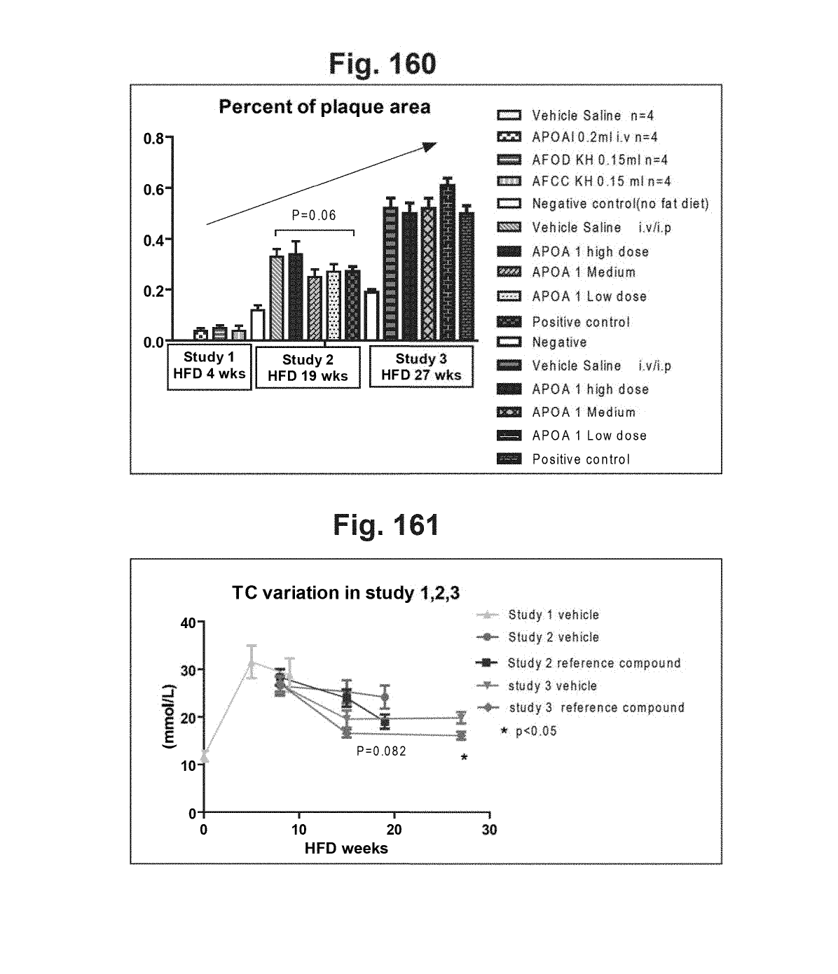

FIG. 160. Comparison of percentage of plaque area in study 1, 2, 3

We also compared percent of plaque area in the study 1, 2 and 3. In study 1, all ApoE ko mice were fed with HFD for 4 weeks and mice were sacrificed at 14 weeks of age. In study 2, all ApoE ko mice were fed with HFD for 19 weeks except the mice in negative control group and all mice were sacrificed at 29 weeks of age. In study 3, the ApoE ko mice were fed with HFD for 27 weeks and sacrificed at 37 weeks. It is apparent that:

1. The plaque area increased steadily with HFD feeding time or aging.

2. The aorta atherosclerosis model in ApoE ko mouse was established successfully.

3. HFD feeding for 10 weeks plus 8 weeks Rx gave best result.

7.10 Comparison of TC Level in Study 1, 2, 3

FIG. 161. Comparison of TC level in study 1, 2, 3

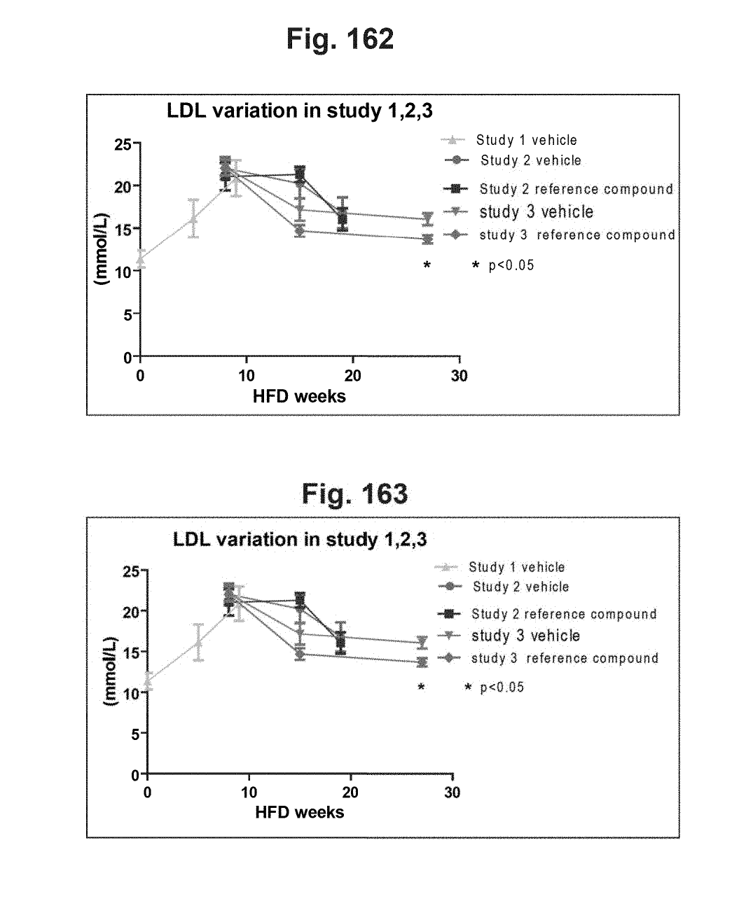

FIG. 162. Comparison of percentage of plaque area in study 1, 2, 3

The TC and LDL values from study 1, 2 and 3 in vehicle and reference groups peaked at week 10, and deceased subsequently during 27 weeks high fat diet feeding. This phenomenon was also observed in relevant literature reports (details can be seen in the report on ppt. version).

7.11. Image of Aorta with Red Oil Staining

One image of aorta stained by oil red from each group was selected and showed below. The branches of artery and the lipid plaques could be observed clearly and the plaques mainly distribute in the aortic root and principal branches of the abdominal aorta. It is consistent with the reference literatures.

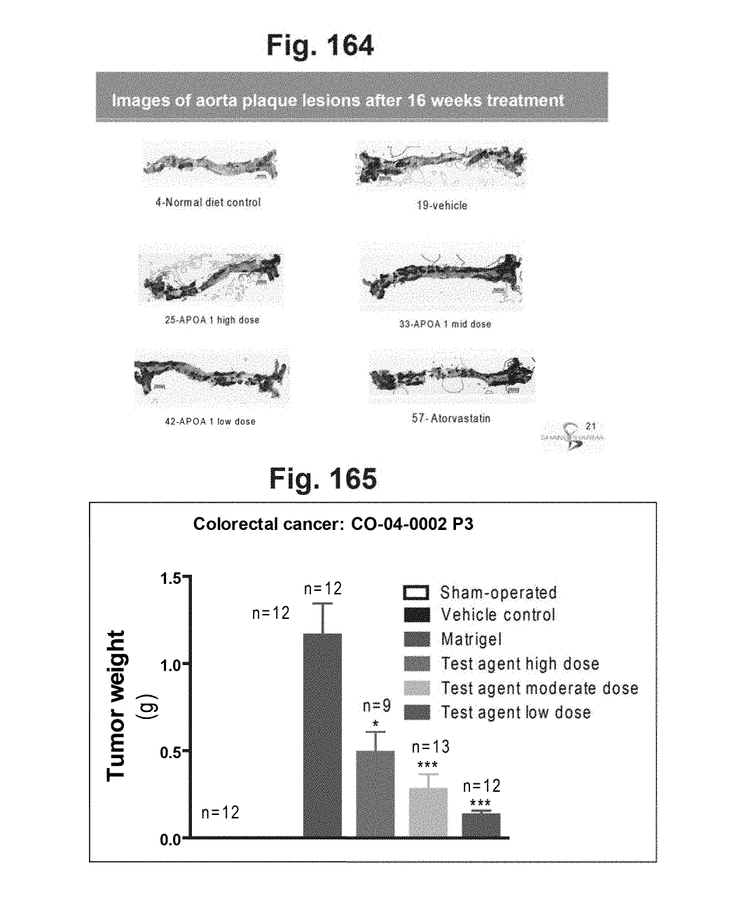

FIG. 164, Images of aorta plaque lesions after 16 weeks treatment

8. Summary and Interpretation
1). Atorvastatin at 40 mg/kg significantly reduced liver/BW ratio, plasma TC, HDL and LDL, but did not affect the plaque lesion area of aorta in ApoE KO mice after 16 weeks treatment.
2). RAAS APOA1 did not affect the lipid profile in ApoE KO mice after 16 weeks treatment.
3). RAAS APOA1 did not reduce the plaque lesion area of aorta in ApoE KO mice after 16 weeks treatment.

Interpretation:
1). The % athero-plaque lesion area reached 50% at the end of 16 week treatment. The 26 week HFD feeding might have made the mice too sick for the test drugs to reverse.
2). Seems 8 weeks treatment gave optimal athero-plaque reduction, as shown by RAAS Study 2 as well as by literature reports.
3). If repeat, suggest to reduce the HFD feeding duration before drug treatment to <6 weeks, and keep the treatment duration to 8 weeks.

9. Conclusion:
1). Atorvastatin at 40 mg/kg significantly reduced plasma TC, HDL and LDL level, liver weight and the ratio of liver/BW, but did not affect the plaque lesion area of aorta in ApoE KO mice after 16 weeks treatment.
2). RAAS antibody APOA1 didn't affect the lipid profile and reduce the plaque lesion of aorta in ApoE KO mice after 16 weeks treatment.
Title: Anti-tumor efficacy of high concentrated fibrinogen enriched a1at thrombin and AFOD in patient-derived tumor xenograft (PDX) models in nude mice.
Description: Patient-derived colorectal tumor xenograft (PDX) model was used to evaluate the anti-cancer efficacy of the high concentrated fibrinogen enriched a1at thrombin and AFOD at different 3 doses. The results showed that high concentrated fibrinogen enriched a1at thrombin and AFOD at all doses significantly inhibited the growth of PDX tumors implanted at 4 different locations of the peritoneum while having minor effects on mice body weights, which indicated high concentrated fibrinogen enriched a1at thrombin and AFOD is a potent anti-cancer agent on colorectal cancer with a limited side effect.
Subject: high concentrated fibrinogen enriched a1at thrombin and AFOD, fibrinogen, thrombin, patient-derived tumor xenograft model, colorectal cancer

Summary

Patient-derived colorectal tumor xenograft (PDX) models (CO-04-0001 or CO-04-0002) were used to evaluate the anti-tumor efficacy of high concentrated fibrinogen enriched a1at thrombin and Afod at 3 doses. PDX tumors (CO-04-0001 or CO-04-0002) were implanted at 4 different locations in peritoneal cavity, and high concentrated fibrinogen enriched a1at thrombin and Afod, or a control agent was applied to peritoneum before and after tumor implantation. 30 days after implantation, the mice were sacrificed and tumors were dissected and weighed. The final tumor weights for all groups were statistically analyzed by one-way ANOVA with the significance level set at 0.05.

The data show that high concentrated fibrinogen enriched a1at thrombin and Afod at all 3 doses exhibits significant inhibitory effects on tumor growth in PDX colorectal cancer model while no significant toxicity was observed, which indicates high concentrated fibrinogen enriched a1at thrombin and Afod is a potential anti-tumor agent in colorectal cancer, warranting further development of the agent for clinical application.

Introduction

The aim of the study was to test anti-tumor efficacy of high concentrated fibrinogen enriched a1at thrombin and Afod in patient-derived colorectal tumor xenograft (PDX) model in nude mice.

The model used in the study was derived from surgically resected, fresh patient tumor tissues. The first generation of the xenograft tumors in mice was termed passage 0 (P0), and so on during continual implantation in mice. The passage of xenograft tumors at P2 (CO-04-0002) or P3 (CO-04-0001) were used in this study.

All the experiments were conducted in the AAALAC-accrediated animal facility in compliance with the protocol approved by the Institutional Animal Care and Use Committee (IACUC).

Methods

Experimental Preparations

Animal Preparation

Female Balb/c nude mice, with a body weight of approximately 20 grams, were obtained from an approved vendor (Sino-British SIPPR/BK Lab. Animal Co. Ltd., Shanghai, China).

Acclimation/Quarantine:

Upon arrival, animals were assessed as to their general health by a member of a veterinary staff or authorized personnel. Animals were acclimated for at least 3 days (upon arrival at the experiment room) before being used for the study.

Animal Husbandry:

Animals were housed in groups during acclimation and individually housed during in-life. The animal room environment was adjusted to the following target conditions: temperature 20 to 25° C., relative humidity 40 to 70%, 12 hours artificial light and 12 hours dark. Temperature and relative humidity was monitored daily.

All animals had access to Certified Rodent Diet (Sino-British SIPPR/BK Lab. Animal Co. Ltd., Shanghai, China) ad libitum. Animals were not fasted prior to the study. Water was autoclaved before provided to the animals ad libitum. Periodic analyses of the water were performed and the results were archived at WuXi AppTec. There were no known contaminants in the diet or water which, at the levels detected expected to interfere with the purpose, conduct or outcome of the study.

Tumor Tissue Preparation

The colorectal xenograft tumor models were established from surgically resected clinical tumor samples. The first generation of the xenograft tumors in mice is termed passage 0 (P0), and so on during continual implantation in mice. The tumor tissues at passage 2 (CO-04-0002) or P3 (CO-04-0001) were used in this study.

Formulation

Test agent: high concentrated fibrinogen enriched a1at thrombin and Afod were provided by RAAS and prepared by RAAS scientist during experiment before use.

Control agent: Matrigel (BD Biosciences; cat. #356234).

Experimental Protocol

Establishment of Xenograft Model and Treatment

Grouping and Treatment

Nude mice were assigned to 6 different groups with 12-17 mice/group and each group received different treatment as shown in Table 9.1.

8 out 17 (9 left) mice in high dose high concentrated fibrinogen enriched a1at thrombin and Afod group died during the first experiment using PDX model CO-04-0002. To make up for the loss of mice in high dose group, 6 additional mice were implanted with tumor fragments collected from model CO-04-0001 and treated with high dose high concentrated fibrinogen enriched a1at thrombin and Afod. So the total mice number in high dose group was 15.

TABLE 9.1
Grouping and the treatment.

| Group | Treatment | N | Remarks |
|---|---|---|---|
| 1 | Sham-operation | 12 | Open up the abdominal cavity and |
| | | | close it with sutures. (No implants) |
| 2 | Vehicle control | 12 | Implant tumor fragments of 20 mm³ in |
| | | | size into 4 corners of abdominal cavity. Close |
| | | | body with sutures. |
| 3 | Matrigel | 12 | Embed tumor fragments of 20 min³ in |
| | | | Matrigel. Implant the tumor fragments into 4 |
| | | | corners of abdominal cavity. Close body |
| | | | with sutures. |
| 4 | 3 ml of high concentrated | 9 + 6 | Spray high concentrated fibrinogen |
| | fibrinogen enriched alat | | enriched a1at thrombin and Afod to cover the |
| | thrombin and Afod (high dose) | | entire peritoneum and the internal organs. |
| | on the peritoneum in | | Implant the tumor fragments of 20 mm³ into |
| | abdominal cavity of nude | | 4 corners of abdominal cavity. Close body with |
| | mice | | sutures. |
| 5 | 2 ml of high concentrated | 12 | Spray high concentrated fibrinogen |
| | fibrinogen enriched a1at | | enriched a1at thrombin and Afod to cover the |
| | thrombin and Afod (moderate | | entire peritoneum and the internal organs. |
| | dose) on the peritoneum in | | Implant the tumor fragments of 20 mm³ into 4 |
| | abdominal cavity of nude mice | | corners of abdominal cavity. Close body with |
| | | | sutures. |
| 6 | 1 ml of high concentrated | 13 | Spray high concentrated fibrinogen |
| | fibrinogen enriched a1at | | enriched a1at thrombin and Afod to cover the |
| | thrombin and Afod (low dose) | | entire peritoneum and the internal organs. |
| | on the peritoneum in | | Implant the tumor fragments of 20 mm³ into |
| | abdominal cavity of nude | | 4 corners of abdominal cavity. Close body with |
| | mice | | sutures. |
| Total | | 76 | |

Experiment Procedures
A. The animal was anesthetized by i.p. injection of sodium pentobarbital at 60-70 mg/kg. Disinfect the abdominal skin of nude mice with 70% ethanol solution. Open up the abdominal wall along the midline of the ventral surface to expose the peritoneal surface.
B. The surgeries for different groups were done according to table 9.1.
C. For groups using test agent, high concentrated fibrinogen enriched a1at thrombin and Afod was then applied on the peritoneal surface.
D. Tumor fragments were implanted at 4 different locations of the peritoneal cavity. The test agent acted as a glue to hold the fragments.
E. The test agent was applied again on the surface of tumor fragments and peritoneum.
F. After the fibrin membrane formed completely, the peritoneal cavity was closed. G. In Matrigel control groups, tumor fragments were embedded into matrigel before implantation.
H. Postoperative cares followed protocol SOP-BEO-0016-1.0.
I. Mice were palpated for tumors 2 weeks after implantation. The ratio of palpable tumors observed in each group was recorded.
J. 30 days after implantation, the mice were sacrificed and tumors were dissected and weighed.
K. The tissues surrounding tumor fragments were also checked to find out whether the tumors had spread to other organ sites within the peritoneal cavity.
L. Pictures of tumor-bearing mice and dissected tumors were taken.
M. If possible, tumor sizes were measured twice per week. Tumor volumes (mm³) are obtained by using the following formula: volume=(W2×L)/2 (W, width; L, length in mm of the tumor).
N. During the experiment, health conditions of mice were observed daily. Body weights of mice were monitored twice per week.

Evaluation of the Anti-Tumor Activity

Health conditions of mice were observed daily. Body weights were measured twice per week during the treatment. Mice were palpated for tumors 2 weeks after implantation. The ratio of palpable tumors observed in each group was recorded.

30 days after treatment, all mice were euthanized with CO₂ and cervical dislocation was followed after respiratory arrest. Routine necropsy was performed to detect any abnormal signs of each internal organ with specific attention to metastases. Each tumor was removed and weighted.

Drugs and Materials

High concentrated fibrinogen enriched a1at thrombin and Afod were provided by RAAS; Matrigel was from BD Biosciences (San Jose, Calif., cat. #356234). Digital caliper was from Sylvac, Switzerland.

Data Analysis

Relative Change of Body Weight (RCBW)

Relative change of body weight (RCBW) was calculated based on the following formula: RCBW (%)=(BWi−BW0)/BW0×100%; BWi was the body weight on the day of weighing and BW0 was the body weight before surgery.

Tumor Weight

Tumors from each mouse were pooled and weighed after sacrificing mice.

Statistical Analysis

Data were expressed as mean±SEM; the difference between the groups was analyzed for significance using one-way ANOVA and Dunnett's test.

Results

Tumor Growth Inhibition

Three weeks after implantation, all 12 mice in vehicle control group showed palpable tumors, while only less than 2 palpable tumors were found in each test agent-treated group. High concentrated fibrinogen enriched a1at thrombin and Afod treatment delayed the appearance of palpable tumors as shown in table 9.2, indicating high concentrated fibrinogen enriched a1at thrombin and Afod inhibited the growth of implanted colorectal tumors in vivo.

Thirty days after implantation, tumors in vehicle control group and matrigel group reached more than 1 g on average. Conversely, tumor weights in test agent high, moderate and low dose groups were 0.49 g (0.35 if when two models are combined), 0.28 g and 0.13 g, respectively. Compared with the vehicle control, high concentrated fibrinogen enriched a1at thrombin and Afod demonstrated significant anti-tumor activities in colorectal cancer PDX model at all 3 doses. The inhibition on tumor growth were shown in FIGS. 26.18 & 26.22 and table 9.2.

Effect on Body weight

Loss of body weight, a sign of toxicity, was not seen in test agent-treated groups, which only showed minor decrease in weight gain. Mortalities were observed within 3 days after surgery and treatment in high dose of test agent group, which may due to the large volume (3 ml) of test agent used in this group.

The effect on body weight was shown in FIG. 26.24 and table 9.3.

Discussion

Patient-derived colorectal tumor xenograft (PDX) model was used to evaluate the anti-cancer efficacy of the high concentrated fibrinogen enriched a1at thrombin and Afod at 3 doses. PDX tumors (CO-04-0001 and CO-04-0002) were implanted at 4 different locations in peritoneal cavity, and high concentrated fibrinogen enriched a1at thrombin and Afod, or a control agent was applied to peritoneum before and after tumor implantation.

Mice were palpated for tumors 2 weeks after implantation. The ratio of palpable tumors observed in each group was recorded. Test agent treatment inhibited the tumor growth as shown by the delayed appearance of palpable tumors. There weeks after implantation, all 12 mice in vehicle control group showed palpable tumors, while only less than 2 palpable tumors were found in each test agent-treated group (Table 9.2).

Thirty days after implantation, the mice were sacrificed and tumors were dissected and weighed. Tumors in vehicle control group and matrigel group reached more than 1 g on average. Conversely, tumor weights in test agent high, moderate and low dose groups were 0.49 g (0.35 when two models are combined), 0.28 g and 0.13 g, respectively. Compared with the vehicle control, high concentrated fibrinogen enriched a1at thrombin and Afod demonstrated significant anti-tumor activities in colorectal cancer PDX model at all 3 doses. Matrigel has been commonly used to facilitate the establishment of human tumor xenografts in rodents. In this study, matrigel group promoted an increase in tumor weight thought the increase was not statistically significant.

Loss of body weight, a sign of toxicity, was not seen in all test agent-treated groups, in which the animals only showed a minor decrease in weight gain compared to sham-operated group. Mortalities observed in test agent high dose group right after the surgery could be due to large volume of test agent (3 ml) used in this group. The mice of vehicle and matrigel groups started to loss body weights 2 weeks after surgery due to the continuously increased tumor volumes.

In summary, the results show that high concentrated fibrinogen enriched a1at thrombin and Afod at all doses significantly inhibits the growth of colorectal tumors in vivo while having minor effects on mice body weight. The results suggest that high concentrated fibrinogen enriched a1at thrombin and Afod is a potent anti-tumor agent in colorectal cancer.

Figures

FIG. 165. Anti-tumor efficacy of high concentrated fibrinogen enriched a1at thrombin and Afod in PDX model CO-04-0002.

Colorectal cancer: CO-04-0002 P3

Tumor weights from model CO-04-0002 were used. Data are expressed as mean±SEM. *<0.05, ***<0.001 vs vehicle group (one-way ANOVA and Dunnett's test).

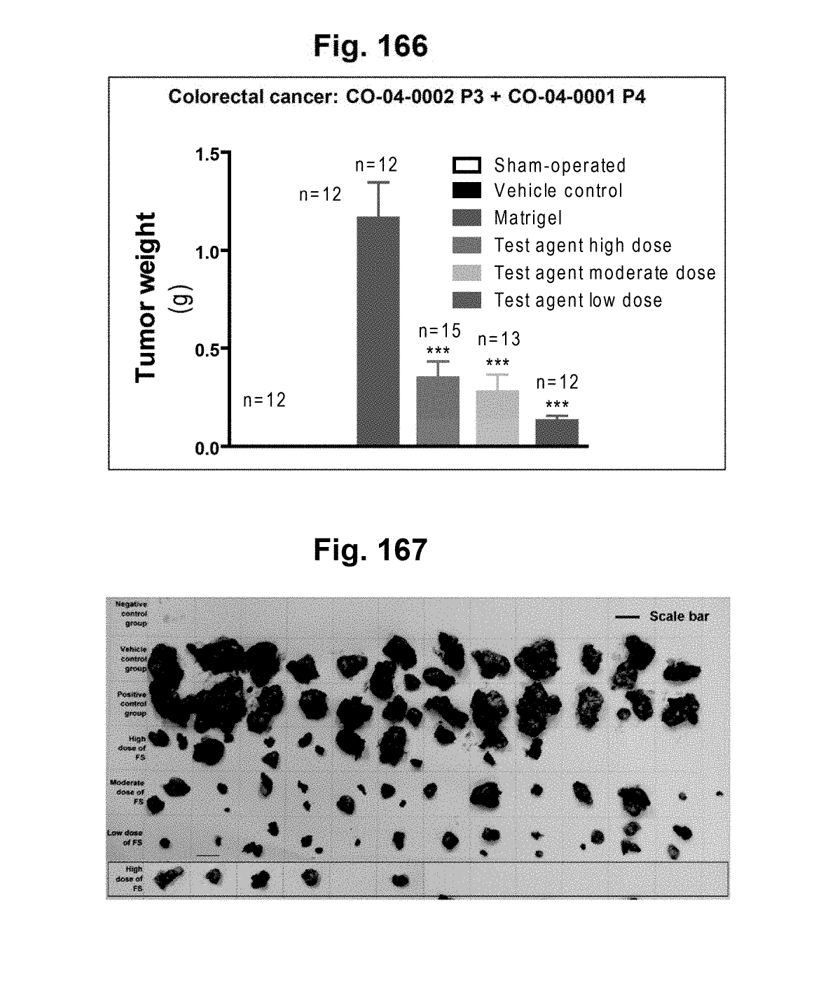

FIG. 166. Anti-tumor efficacy of high concentrated fibrinogen enriched a1at thrombin and Afod in PDX model CO-04-0002 and CO-04-0001.

Colorectal cancer: CO-04-0002 P3+CO-04-0001 P4

Tumor weights of 6 mice from model CO-04-0001 were combined with the data from model CO-04-0002. There were 15 mice in total in high dose of test agent group. Data are expressed as mean±SEM. *<0.05, ***<0.001 vs vehicle group (one-way ANOVA and Dunnett's test).

FIG. 167. Photographs of tumors dissected from abdominal cavity of each group.

Tumors from each mouse were pooled and weighed. The tumors in frame were from model CO-04-0002 (upper panels) and the rest were form model CO-04-0001 (bottom panel). Scale bar, 1 cm.

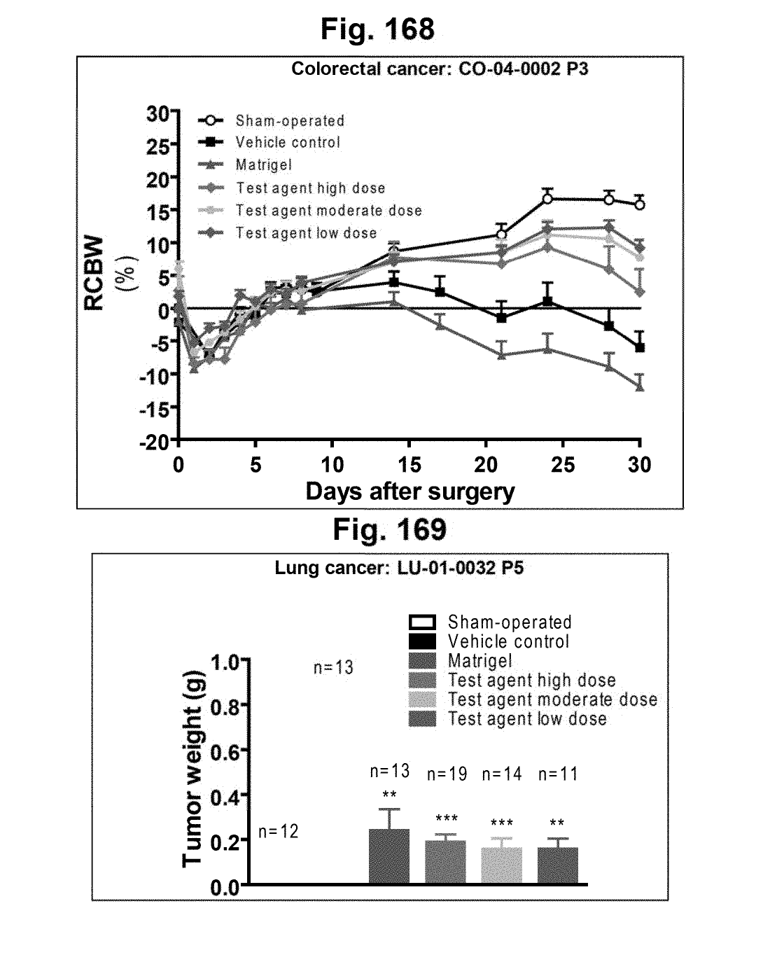

FIG. 168. Relative change of body weight (%) of different groups.

Data are expressed as mean±SEM. Relative change of body weight (RCBW) was calculated based on the following formula: RCBW (%)=(BWi−BW0)/BW0×100%; BWi was the body weight on the day of weighing and BW0 was the body weight before surgery.

Confidential

Tables

TABLE 9.2
Ratios of palpable tumors observed in each group.

| Days after | | | | | | | | |
|---|---|---|---|---|---|---|---|---|
| surgery | 15 | 16 | 17 | 18 | 20 | 21 | 24 | 28 |
| Sham-operated | 0/12 | 0/12 | 0/12 | 0/12 | 0/12 | 0/12 | 0/12 | 0/12 |
| group | | | | | | | | |
| vehicle control | 0/12 | 1/12 | 4/12 | 4/12 | 8/12 | 12/12 | 12/12 | 12/12 |
| group | | | | | | | | |
| Matrigel | 1/12 | 3/12 | 5/12 | 5/12 | 5/12 | 8/12 | 11/12 | 12/12 |
| high dose of | 0/9 | 0/9 | 0/9 | 0/9 | 0/9 | 0/9 | 0/9 | 5/9 |
| test agent | | | | | | | | |
| moderate dose of | 0/13 | 0/13 | 1/13 | 1/13 | 1/13 | 2/13 | 2/13 | 5/13 |
| test agent | | | | | | | | |
| low dose of | 0/12 | 0/12 | 1/12 | 1/12 | 1/12 | 1/12 | 2/12 | 7/12 |
| test agent | | | | | | | | |

Mice were palpated for tumors at 15, 16, 17, 18, 20, 21, 24, 28 days after implantation.
The ratios of palpable tumors observed in each group were recorded.

TABLE 9.3
Relative change of body weight (%) of different groups.

| Days | | | | | | | | | | | | | | | |
|---|---|---|---|---|---|---|---|---|---|---|---|---|---|---|---|
| after | | | | | | | | | | | | | | | |
| surgery | 0 | | 1 | 2 | 3 | 4 | 5 | 6 | 7 | 8 | 9 | 14 | 21 | 24 | 28 |
| Group | RCB | | RCB | RCB | RCB | RCB | RCB | RCB | RCB | RCB | RCB | RCB | RCB | RCB | RCB |
| | W (%) | | W (%) | W (%) | W (%) | W (%) | W (%) | W (%) | W (%) | W (%) | W (%) | W (%) | W (%) | W (%) | W (%) |
| Sham- | Mean | 0.00 | −7.41 | −3.46 | −0.73 | −1.24 | 3.08 | 3.18 | 2.84 | 2.45 | 8.67 | 11.20 | 16.61 | 16.46 | 15.70 |
| operated | SD | 0.00 | 2.98 | 3.03 | 3.03 | 4.19 | 2.94 | 3.18 | 3.18 | 4.21 | 4.21 | 5.70 | 5.31 | 4.87 | 5.07 |
| | SEM | 0.00 | 0.86 | 0.87 | 0.88 | 1.21 | 0.85 | 0.92 | 0.92 | 1.21 | 1.21 | 1.65 | 1.53 | 1.41 | 1.46 |
| Vehicle | Mean | −2.14 | −7.06 | −4.16 | −2.12 | −0.99 | 2.37 | 2.24 | 3.38 | 2.55 | 3.92 | −1.48 | 1.02 | −2.70 | −6.01 |
| control | SD | 0.87 | 2.62 | 2.93 | 3.82 | 3.99 | 4.06 | 4.10 | 3.85 | 4.34 | 5.74 | 8.75 | 9.95 | 9.36 | 8.72 |
| | SEM | 0.25 | 0.76 | 0.85 | 1.10 | 1.15 | 1.17 | 1.18 | 1.11 | 1.25 | 1.66 | 2.53 | 2.87 | 2.70 | 2.52 |
| Matrigel | Mean | −1.97 | −9.20 | −7.41 | −4.43 | −3.47 | 0.62 | 0.09 | | 1.50 | −0.29 | | −7.17 | −6.25 | −8.92 |
| | SD | 1.14 | 2.37 | 3.60 | | 2.17 | 2.74 | 2.46 | 3.07 | 3.35 | 4.95 | 7.26 | 8.33 | 7.24 | 6.50 |
| | SEM | 0.33 | 0.68 | 1.04 | 0.73 | 0.63 | 0.79 | 0.71 | 0.89 | 0.97 | 1.43 | 2.10 | 2.40 | 2.09 | 1.88 |
| High | Mean | 2.66 | −8.41 | −7.80 | −7.78 | −3.48 | −2.09 | −0.26 | 0.50 | 0.73 | 7.71 | 6.72 | 9.28 | 5.90 | 2.48 |
| dose | SD | 6.60 | 2.76 | 4.29 | 5.40 | 3.19 | 5.68 | 6.05 | 6.52 | 5.77 | 7.22 | 7.93 | 8.90 | 10.53 | 10.39 |
| Of test | SEM | 2.20 | 0.92 | 1.43 | 1.80 | 1.06 | 1.89 | 2.02 | 2.17 | 1.92 | 2.41 | 2.64 | 2.97 | 3.51 | 3.46 |
| agent | | | | | | | | | | | | | | | |
| Mod- | Mean | 5.95 | −6.73 | −5.23 | −3.70 | −1.70 | 0.30 | 2.37 | 2.55 | 2.66 | 7.00 | 8.46 | 11.16 | 10.55 | 7.68 |
| erate | SD | 4.04 | 2.13 | 2.43 | 4.38 | 4.61 | 5.08 | 4.15 | 5.29 | 5.85 | 5.58 | 7.03 | 7.98 | 10.25 | 9.57 |
| dose of | SEM | 1.12 | 0.59 | 0.67 | 1.22 | 1.28 | 1.41 | 1.15 | 1.47 | 1.62 | 1.55 | 1.95 | 2.21 | 2.84 | 2.66 |
| test | | | | | | | | | | | | | | | |
| agent | | | | | | | | | | | | | | | |
| Low | Mean | 1.82 | −5.27 | −3.04 | −2.75 | 1.93 | 1.00 | 2.86 | 2.11 | 3.85 | 7.08 | 7.08 | 12.03 | 12.27 | 9.18 |
| Dose | SD | 2.74 | 2.24 | 2.55 | 2.53 | 2.90 | 2.15 | 3.00 | 2.89 | 3.17 | 3.78 | 3.78 | 3.65 | 3.77 | 4.16 |
| of test | SEM | 0.79 | 0.65 | 0.74 | 0.73 | 0.84 | 0.62 | 0.87 | 0.84 | 0.92 | 1.09 | 1.09 | 1.05 | 1.09 | 1.20 |
| agent | | | | | | | | | | | | | | | |

Relative change of body weight (RCBW) was calculated based on the following formula: RCBW (%)=(BWi−BW0)/BW0×100%;

BWi was the body weight on the day of weighing and BW0 was the body weight before surgery.
Title: Anti-tumor efficacy of high concentrated fibrinogen enriched a1at thrombin and Afod in a patient-derived tumor xenograft (PDX) model of lung cancer in nude mice.
Description: Patient-derived tumor xenograft (PDX) model of lung cancer was used to evaluate the anti-cancer efficacy of high concentrated fibrinogen enriched a1at thrombin and Afod at different 3 doses. The results showed that high concentrated fibrinogen enriched a1at thrombin and afod at all doses significantly inhibited the growth of PDX tumors implanted at 4 different locations of the peritoneum while having minor effects on mice body weights, which indicates high concentrated fibrinogen enriched a1at thrombin and Afod is a potent anti-cancer agent on lung cancer with a limited side effect.
Subject: high concentrated fibrinogen enriched a1at thrombin and Afod, patient-derived tumor xenograft model, lung cancer

Summary

Patient-derived tumor xenograft (PDX) model of lung cancer (LU-01-0032) was used to evaluate the anti-tumor efficacy of high concentrated fibrinogen enriched a1at thrombin and Afod at 3 doses. PDX tumors (LU-01-0032) were implanted at 4 different locations in peritoneal cavity, and high concentrated fibrinogen enriched a1at thrombin and Afod or a control agent was applied to peritoneum before and after tumor implantation. Forty five days after implantation, the mice were sacrificed and tumors were removed and weighed. The final tumor weights for all groups were statistically analyzed by one-way ANOVA with the significance level set at 0.05.

The data show that high concentrated fibrinogen enriched a1at thrombin and Afod at all 3 doses exhibits significant inhibitory effects on tumor growth in the lung cancer model while no significant toxicity was observed, which indicates high concentrated fibrinogen enriched a1at thrombin and Afod was a potential anti-tumor agent in lung cancer, warranting further development of high concentrated fibrinogen enriched a1at thrombin and Afod for clinical application.

1. Introduction

The aim of the study was to test anti-tumor efficacy of high concentrated fibrinogen enriched a1at thrombin and Afod in patient-derived lung tumor xenograft (PDX) model in nude mice.

The model used in the study was derived from surgically resected, fresh patient tumor tissues. The first generation of the xenograft tumors in mice was termed passage 0 (P0), and so on during continual implantation in mice. The passage of xenograft tumors at P5 (LU-01-0032) were used in this study.

All the experiments were conducted in the AAALAC-accrediated animal facility in compliance with the protocol approved by the Institutional Animal Care and Use Committee (IACUC).

2. Methods

Mental Preparations

2.1.1. Animal Preparation

Female Balb/c nude mice, with a body weight of approximately 20 grams, were obtained from an approved vendor (Sino-British SIPPR/BK Lab. Animal Co. Ltd., Shanghai, China).

Acclimation/Quarantine:

Upon arrival, animals were assessed as to their general health by a member of a veterinary staff or authorized personnel. Animals were acclimated for at least 3 days (upon arrival at the experiment room) before being used for the study.

Animal Husbandry:

Animals were housed in groups during acclimation and individually housed during in-life. The animal room environment was adjusted to the following target conditions: temperature 20 to 25° C., relative humidity 40 to 70%, 12 hours artificial light and 12 hours dark. Temperature and relative humidity was monitored daily.

All animals had access to Certified Rodent Diet (Sino-British SIPPR/BK Lab. Animal Co. Ltd., Shanghai, China) ad libitum. Animals were not fasted prior to the study. Water was autoclaved before provided to the animals ad libitum. Periodic analyses of the water were performed and the results were archived at WuXi AppTec. There were no known contaminants in the diet or water which, at the levels detected expected to interfere with the purpose, conduct or outcome of the study.

2.1.2. Tumor Tissue Preparation

The lung xenograft tumor models were established from surgically resected clinical tumor samples. The first generation of the xenograft tumors in mice is termed passage 0 (P0), and so on during continual implantation in mice. The tumor tissues at passage 5 (LU-01-0032) were used in this study.

2.1.3. Formulation

High concentrated fibrinogen enriched a1at thrombin and Afod were provide by RAAS and prepared by RAAS scientist during experiment before use.

Matrigel (BD Biosciences; cat. #356234).

2.2. Experimental Protocol

2.2.1. Establishment of Xenograft Model and Treatment

Grouping and Treatment

Nude mice were assigned to 6 different groups with 11-19 mice/group and each group received different treatments as shown in Table 1.

TABLE 1
Grouping and the treatment.

| Group | Treatment | N | Remarks |
|---|---|---|---|
| 1 | Sham-operation | 12 | Open up the abdominal cavity and close it with |
| | | | sutures. (No implants) |
| 2 | Vehicle control | 13 | Implant tumor fragments of 20 mm³ in size into |
| | | | 4 corners of abdominal cavity. Close body with |
| | | | sutures. |
| 3 | Matrigel | 13 | Embed tumor fragments of 20 mm³ in Matrigel. |
| | | | Implant the tumor fragments into 4 corners of |
| | | | abdominal cavity. Close body with sutures. |
| 4 | 3 ml high concentrated | 19 | Spray high concentrated fibrinogen enriched a1at |
| | fibrinogen enriched a1at | | thrombin and Afod to cover the entire |
| | thrombin and Afod (high dose) | | peritoneum and the internal organs. Implant the |
| | on the peritoneum in abdominal | | tumor fragments of 20 mm³ into 4 corners of |
| | cavity of nude mice | | abdominal cavity. Close body with sutures. |
| 5 | 2 ml high concentrated | 14 | Spray high concentrated fibrinogen enriched a1at |
| | fibrinogen enriched a1at | | thrombin and Afod to cover the entire |
| | thrombin and Afod (moderate | | peritoneum and the internal organs. Implant the |
| | dose) on the peritoneum in | | tumor fragments of 20 mm³ into 4 corners of |
| | abdominal cavity of nude mice | | abdominal cavity. Close body with sutures. |
| 6 | 1 ml high concentrated | 11 | Spray high concentrated fibrinogen enriched a1at |
| | fibrinogen enriched a1at | | thrombin and Afod to cover the entire |
| | thrombin and Afod (low dose) | | peritoneum and the internal organs. Implant the |
| | on the peritoneum in abdominal | | tumor fragments of 20 mm³ into 4 corners of |
| | cavity of nude mice | | abdominal cavity. Close body with sutures. |
| Total | | 82 | |

Experiment Procedures
A. Measured the body weight of each mouse before surgery.
B. The animal was anesthetized by i.p. injection of sodium pentobarbital at 60-70 mg/kg. Disinfect the abdominal skin of nude mice with 70% ethanol solution. Open up the abdominal wall along the midline of the ventral surface to expose the peritoneal surface.
C. The surgeries for different groups were done according to table 1.
D. For groups using test agent high concentrated fibrinogen enriched a1at thrombin and Afod, the test agent was then applied on the peritoneal surface.
E. Tumor fragments were implanted at 4 different locations of the peritoneal cavity. The test agent acted as a glue to hold the fragments.
F. The test agent high concentrated fibrinogen enriched a1at thrombin and Afod was applied again on the surface of tumor fragments and peritoneum.
G. After the fibrin membrane formed completely, the peritoneal cavity was closed.
H. In Matrigel control groups, tumor fragments were embedded into matrigel before implantation.
I. Postoperative cares followed protocol SOP-BEO-0016-1.0.
J. Mice were palpated for tumors 2 weeks after implantation. The ratio of palpable tumors observed in each group was recorded.
K. Forty five days after implantation, the mice were sacrificed and tumors were dissected and weighed.
L. The tissues surrounding tumor fragments were also checked to find out whether the tumors had spread to other organ sites within the peritoneal cavity.
M. Pictures of tumor-bearing mice and dissected tumors were taken.
N. If possible, tumor sizes were measured twice per week. Tumor volumes (mm³) are obtained by using the following formula: volume=(W2×L)/2 (W, width; L, length in mm of the tumor).
O. During the experiment, health conditions of mice were observed daily. Body weights of mice were monitored twice per week.

2.2.2. Evaluation of the Anti-Tumor Activity

Health conditions of mice were observed daily. Body weights were measured twice per week during the treatment. Mice were palpated for tumors 2 weeks after implantation. The ratio of palpable tumors observed in each group was recorded. 45 days after treatment, all mice were euthanized with CO₂ and cervical dislocation was followed after respiratory arrest. Routine necropsy was performed to detect any abnormal signs of each internal organ with specific attention to metastases. Each tumor was removed and weighted.

2.3. Drugs and Materials

High concentrated fibrinogen enriched a1at thrombin and Afod were provided by RAAS; Matrigel was from BD Biosciences (San Jose, Calif., cat. #356234).

Digital caliper was from Sylvac, Switzerland.

2.4. Data Analysis

2.4.1. Relative Change of Body Weight (RCBW)

Relative change of body weight (RCBW) was calculated based on the following formula: RCBW (%)=(BWi−BW0)/BW0×100%; BWi was the body weight on the day of weighing and BW0 was the body weight before surgery.

2.4.2. Tumor Weight

Tumors from each mouse were pooled and weighed after sacrificing mice.

2.4.3. Statistical Analysis

Data were expressed as mean±SEM; the difference between the groups was analyzed for significance using one-way ANOVA and Dunnett's test.

3. Results

3.1. Tumor Growth Inhibition

Four weeks after implantation, 9 out of 13 mice in vehicle control group showed palpable tumors, while only less than 5 palpable tumors were found in each high concentrated fibrinogen enriched a1at thrombin and Afod-treated group. High concentrated fibrinogen enriched a1at thrombin and Afod treatment delayed the appearance of palpable tumors as shown in table 2, indicating high concentrated fibrinogen enriched a1at thrombin and Afod inhibited the growth of implanted lung tumors in vivo. After sacrificing the mice, tumors were found in all the mice in vehicle control group, while some tumors completely regressed in several high concentrated fibrinogen enriched a1at thrombin and Afod-treated mice (FIG. 3).

Forty-five days after implantation, tumors in vehicle control group reached more than 0.7 g on average. Conversely, tumor weights in high concentrated fibrinogen enriched a1at thrombin and Afod high, moderate and low dose groups were 0.19 g, 0.16 g and 0.16 g, respectively. Compared with the vehicle control, high concentrated fibrinogen enriched a1at thrombin and Afod demonstrated significant anti-tumor activities in lung cancer PDX model at all 3 doses (FIG. 1˜2).

The inhibition on tumor growth were shown in FIG. 1˜3 and table 2.

3.2. Effect on Body Weight

Loss of body weight, a sign of toxicity, was not seen in high concentrated fibrinogen enriched a1at thrombin and Afod-treated groups, indicating the test agent has no/little side effects.

The effect on body weight was shown in FIG. 4 and table 3.

4. Discussion

Patient-derived tumor xenograft (PDX) model of lung cancer was used to evaluate the anti-cancer efficacy of the high concentrated fibrinogen enriched a1at thrombin and Afod at 3 doses. PDX tumors (LU-01-0032) were implanted at 4 different locations in peritoneal cavity, and high concentrated fibrinogen enriched a1at thrombin and Afod or a control agent was applied to peritoneum before and after tumor implantation.

Mice were palpated for tumors 2 weeks after implantation. The ratio of palpable tumors observed in each group was recorded. High concentrated fibrinogen enriched a1at thrombin and Afod treatment inhibited the tumor growth as shown by the delayed appearance of palpable tumors and decreased tumor incidence. Four weeks after implantation, 9 out of 13 mice in vehicle control group showed palpable tumors, while only less than 5 palpable tumors were found in each high concentrated fibrinogen enriched a1at thrombin and Afod-treated group (Table 2).

Forty-five days after implantation, the mice were sacrificed and tumors were dissected and weighed. After sacrificing the mice, tumors were found in all the mice in vehicle control group, while some tumors completely regressed in several high concentrated fibrinogen enriched a1at thrombin and Afod-treated mice. Tumors in vehicle control group reached more than 0.7 g on average. Conversely, tumor weights in high concentrated fibrinogen enriched a1at thrombin and Afod high, moderate and low dose groups were 0.19 g, 0.16 g and 0.16 g, respectively. Compared with the vehicle control, high concentrated fibrinogen enriched a1at thrombin and Afod demonstrated significant anti-tumor activities in lung cancer PDX model at all 3 doses. Matrigel has been commonly used to facilitate the establishment of human tumor xenografts in rodents. In this study, matrigel group also showed a significant inhibitory effect on tumor weight.

Loss of body weight, a sign of toxicity, was not seen in all high concentrated fibrinogen enriched a1at thrombin and Afod-treated groups, indicating the test agent has no/little side effects.

In summary, the results show that high concentrated fibrinogen enriched a1at thrombin and Afod at all doses significantly inhibits the growth of lung tumors in vivo while having minor effects on mice body weight. The results suggest that high concentrated fibrinogen enriched a1at thrombin and Afod is a potent anti-tumor agent in lung cancer.

5. Figures

FIG. 169. Anti-tumor efficacy of high concentrated fibrinogen enriched a1at thrombin and Afod in PDX model LU-01-0032.

Tumor weights from model LU-01-0032 were used. Data are expressed as mean±SEM. *<0.05, **<0.01, ***<0.001 vs vehicle group (one-way ANOVA and Dunnett's test).

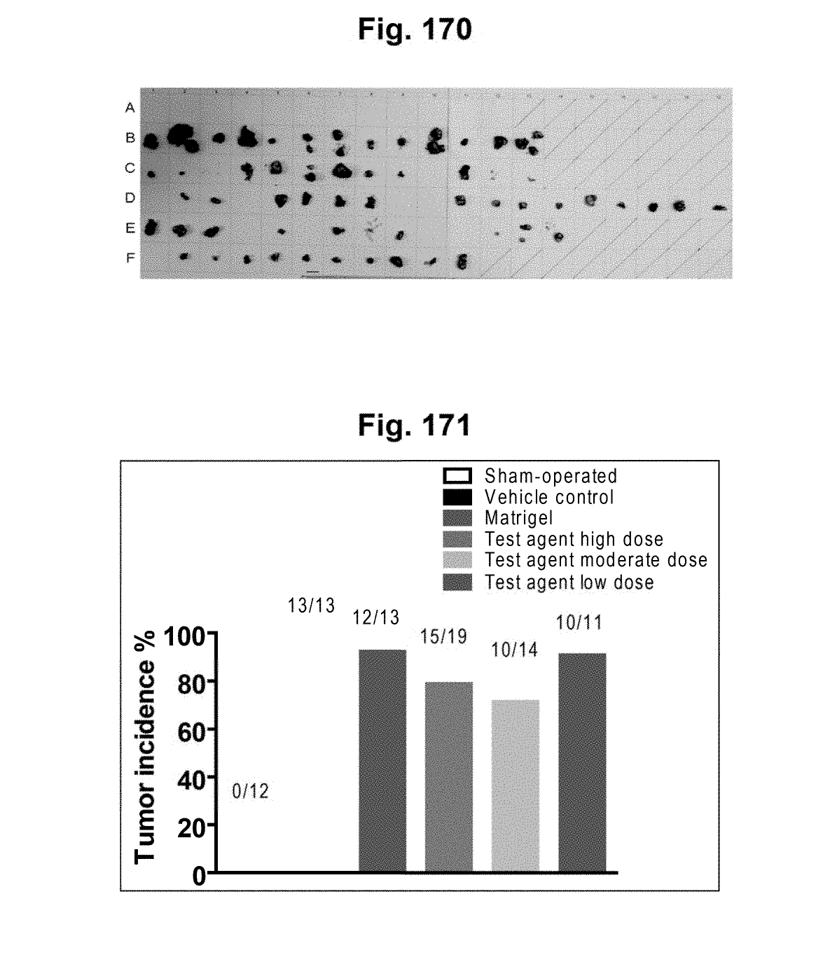

FIG. 170. Photographs of tumors dissected from abdominal cavity of each group.

Tumors from each mouse of model LU-01-0032 were pooled and weighed. Scale bar, 1 cm. A, sham-operated; B, vehicle control; C, matrigel; D, test agent high dose; E, test agent moderate dose; F, test agent low dose.

FIG. 171. Ratios of mice with palpable tumors observed in each group.

After sacrificing the mice, the tumors from each mouse of model LU-01-0032 were pooled and the ratios of mice bearing tumors in each group were recorded.

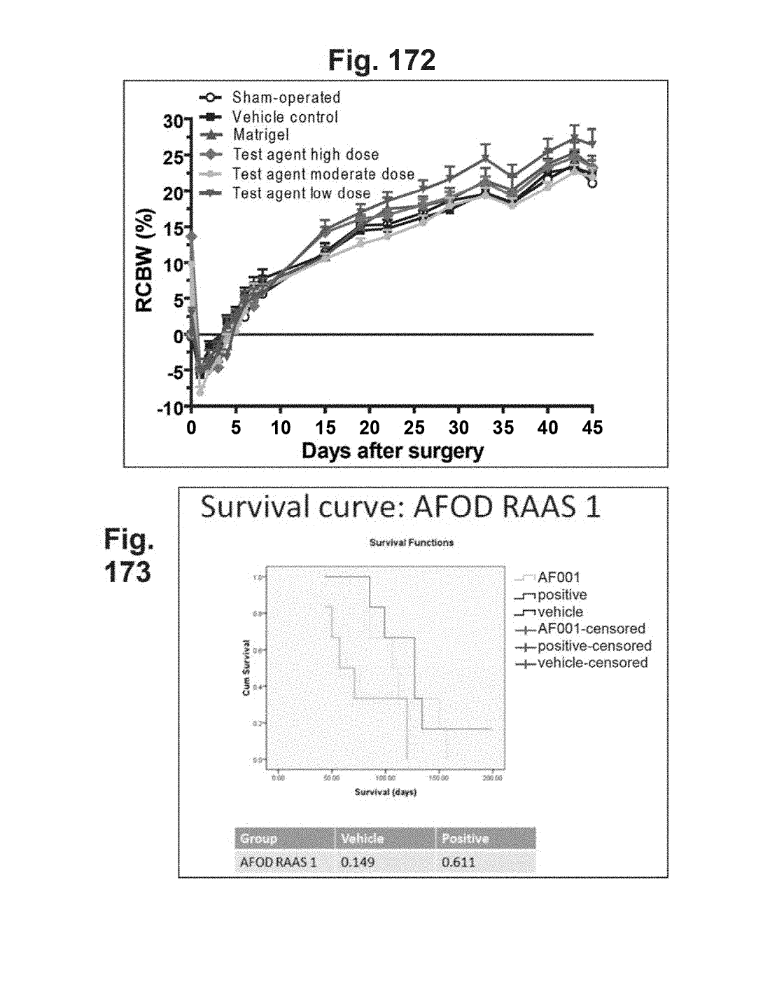

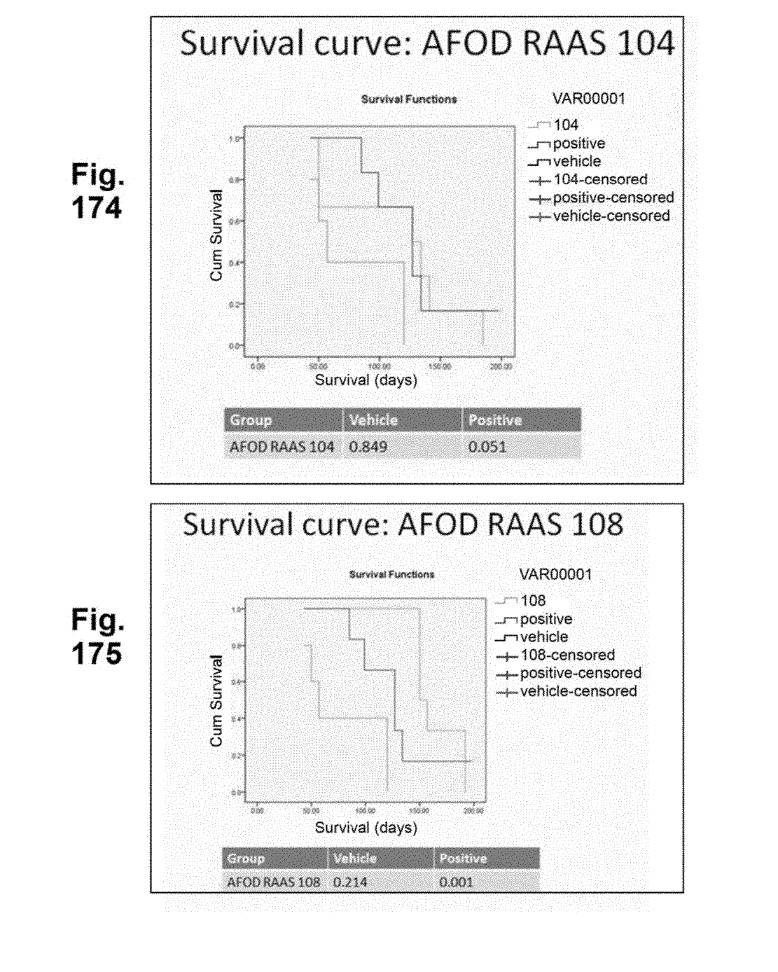

FIG. 172. Relative change of body weight (%) of different groups.

Data are expressed as mean±SEM. Relative change of body weight (RCBW) was calculated based on the following formula: RCBW (%)=(BWi−BW0)/BW0×100%; BWi was the body weight on the day of weighing and BW0 was the body weight before surgery.

6. Tables

Relative change of body weight (RCBW) was calculated based on the following formula: RCBW (%)=(BWi−BW0)/BW0×100%; BWi was the body weight on the day of weighing and BW0 was the body weight before surgery.

| | |
|---|---|
| Title: | In Vivo Test of Efficacy of FS in the Treatment of |
| | BEL-7404 Peritoneal Implantation Model |
| Description: | BEL-7404 peritoneal Implantation hepatic cancer |
| | model was used to evaluate the anti-cancer efficacy |
| | of the FS at 2 ml/mouse. The results showed that FS |
| | had inhibition on tumor growth. |
| Subject: | FS, BEL-7404, hepatic cancer |
| Project ID: | RAAS-20130425 |

Summary

BEL-7404 peritoneal Implantation hepatic cancer model was used to evaluate the anti-cancer efficacy of FS at 2 ml/mouse. On day 21 after implantation, all mice in vehicle group and positive group exhibited palpable tumors, while no mice in FS group exhibited any palpable tumors. On day 28 after implantation, the tumor in vehicle group reached 0.88 g, while FS group was only 0.06 g. On day 52 after implantation, Five mice treated with FS did not exhibit any palpable tumor. The results showed that FS had inhibition on tumor growth.

No body weight loss and toxicity were found in FS-treated groups, which showed FS had no side effect.

Introduction

The objective of the research is to evaluate the in vivo efficacy of FS in the treatment of a hepatic cancer model.

All the experiments were conducted in the AAALAC-accrediated animal facility in compliance with the protocol approved by the Institutional Animal Care and Use Committee (IACUC).

Methods

Experimental Preparations

Animal Preparation

Female Balb/c nude mice, with a body weight of approximately 20 grams, were obtained from an approved vendor (Shanghai BK Laboratory Animal Co., LTD., Shanghai, China).

Acclimation/Quarantine:

Upon arrival, animals were assessed as to their general health by a member of a veterinary staff or authorized personnel. Animals were acclimated for at least 3 days (upon arrival at the experiment room) before being used for the study.

Animal Husbandry:

Animals were housed in groups during acclimation and individually housed during in-life. The animal room environment was adjusted to the following target conditions: temperature 20 to 25° C., relative humidity 40 to 70%, 12 hours artificial light and 12 hours dark. Temperature and relative humidity was monitored daily.

All animals had access to Certified Rodent Diet (Shanghai BK Laboratory Animal Co., LTD., Shanghai, China) ad libitum. Animals were not fasted prior to the study. Water was autoclaved before provided to the animals ad libitum. Periodic analyses of the water were performed and the results were archived at WuXi AppTec. There were no known contaminants in the diet or water which, at the levels detected expected to interfere with the purpose, conduct or outcome of the study.

Cell Culture:

The BEL-7404 tumor cells were maintained in vitro as a monolayer culture in RPMI 1640 medium supplemented with 10% heat inactivated fetal bovine serum, 100 U/ml penicillin and 100 μg/ml streptomycin, and L-glutamine (2 mM) at 37° C. in an atmosphere of 5% CO₂ in air. The tumor cells were routinely subcultured twice weekly by trypsin-EDTA treatment. The cells growing in an exponential growth phase were harvested and counted for tumor inoculation. When the average tumor volume reached 1000 mm³, sacrificed the tumor bearing mice and removed the tumor for orthotopic implantation.

Formulation

FS were provide by RAAS and prepared by RAAS scientist during experiment before use.

Matrigel (BD Biosciences; cat. #356234)

Experimental Protocol

Establishment of Xenograft Model and Treatment

Grouping and Treatment

Nude mice were assigned to 6 different groups with 3 mice/group and each group received different treatment as shown in Table 1.

TABLE 1
Grouping and the treatment

| Group | Treatment | N | Remarks |
|---|---|---|---|
| 1 | Vehicle control | 3 | Implant a tumor fragment of 40 mm³ into the abdominal |
| | | | cavity. Close body with sutures |
| 2 | Positive control | 3 | Embed a tumor fragment of 40 mm³ in Matrigel. Implant the |
| | | | tumor fragment into abdominal cavity. Close body with |
| | | | sutures |
| 3 | 3-FS (2 ml) + | 3 | Spray FS to cover the entire peritoneum and the internal |
| | sorafenib | | organs. Implant a tumor fragment of 40 mm³ into abdominal |
| | | | cavity, spread some sorafenib on the tumor. Spray FS to cover |
| | | | the tumor. Close body with sutures |
| 4 | FS alone (2 ml) | 3 | Spray FS to cover the entire peritoneum and the internal |
| | | | organs. Implant a tumor fragment of 40 mm³ into abdominal |
| | | | cavity. Spray FS to cover the tumor. Close body with sutures |
| 5 | FS alone (2 ml) | 3 | Spray FS to cover the entire peritoneum and the internal |
| | | | organs. Implant a tumor fragment of 40 mm³ into abdominal |
| | | | cavity. Spray FS to cover the tumors. Close body with sutures |
| 6 | FS + oral | 3 | Spray FS to cover the entire peritoneum and the internal |
| | application (2 ml) | | organs. Implant a tumor fragment of 40 mm³ into abdominal |
| | | | cavity. Spray FS to cover the tumors. Close body with sutures. |
| | | | After implantation, the mice were treated according to table2 |
| Total | | 18 | |

TABLE 2
treatment schedule in group 6

| Medicine | Concentration | Route | Dosage | Schedule |
|---|---|---|---|---|
| kh afcc | NA | Drink | NA | 22 h on 2 h off (D0-D8) |
| kh afcc | NA | PO | 0.4 ml | BID (D9-D31) |
| KH-R1 | NA | Drink | NA | 24 h (D32-D42) |
| 5bp121 | NA | IP | 0.5 ml | QD (D43-D54) |

The doses of group6 was changed during the experiment as requested by the sponsor

Experiment Procedures
A. Establishment of cell line xenograft tumor model in female Balb/c nude mice: cells were injected at 3×10⁶ cells per animal subcutaneously.
B. When the tumor reached about 1000 mm³, sacrifice these animals, removed tumor for peritoneal implantation.
C. Fifteen mice were anesthetized by i.p. injection of sodium pentobarbital at 60-70 mg/kg. The animal skin was sterilized with ethanol solution. Then the body wall was opened and the peritoneal surface was exposed.
D. The test agent was then applied on the peritoneal surface, including both sides. The amount of the test agent was listed on the above table.
E. Tumor fragment was implanted into the peritoneum. The test agent acted as a glue to hold the fragment.
F. After the fibrin membrane formed completely, the peritoneal cavity was closed and sutured.
G. In the positive control group, tumor fragment was embedded in Matrigel.
H. Body weight was measured upon completion of surgery.
I. Postoperative cares followed protocol SOP-BEO-0016-1.0.
J. Group 1, 2 and 4 were sacrificed 4 weeks after implantation with tumor growing in all vehicle and positive control. Group 3, 5 and 6 were for long term study for 54 days.
K. During the period of the experiment, health conditions of mice were observed daily. Body weight of mice was monitored twice per week.
L. Tumor sizes were measured twice per week when tumor could be measured. Tumor volumes (mm³) were obtained by using the following formula: volume=(W²×L)/2 (W, width; L, length in mm of the tumor)
M. Mice, which showed a significant loss of body weight (>20%), or which were unable to eat or drink, or exhibit ulceration on the skin/tumor, or the tumor size reaches 2,000 mm³, were euthanized immediately to minimize the pain and distress. Such actions need to notify the sponsor within 24 hrs (48 hrs during the weekends).

Evaluation of the Anti-Tumor Activity

Health conditions of mice were observed daily. Body weights were measured twice per week during the treatment. The ratio of palpable tumors observed in each group was recorded. Group 1, 2 and 4 were sacrificed 4 weeks after implantation with tumor growing in all vehicle or positive control. Group 3, 5 and 6 were for long term study for 54 days. Each tumor and FS was removed and weighed.

Drugs and Materials

FS was provided by RAAS; Matrigel was from BD Biosciences (San Jose, Calif., cat. #356234).

Digital caliper was from Sylvac, Switzerland.

Data Analysis

Ratios of Palpable Tumors Observed in Different Groups

Record the palpable tumors of each mouse observed as an indicator of efficacy

Tumor Weight

Group 1, 2 and 4 were sacrificed 4 weeks after implantation with tumor growing in all vehicle or positive control. Group 3, 5 and 6 were for long term study for 54 days. Each tumor was removed and weighted.

Relative Change of Body Weight (RCBW)

Relative change of body weight (RCBW) was calculated based on the following formula: RCBW (%)=(BWi−BW0)/BW0×100%; BWi was the body weight on the day of weighing and BW0 was the body weight before surgery.

Statistical Analysis

Data was expressed as mean±S.E.;

Results

Ratios of Palpable Tumors Observed in Different Groups

On day 21 after implantation, all mice in vehicle group and all mice in positive group exhibited palpable tumor, while no mice in FS groups exhibited any palpable tumors. On 52 days after implantation, five mice treated with FS did not exhibit any palpable tumor. The summary of ratios of palpable tumors observed in different groups was shown in table 3.

Tumor Weight

On day 28 after implantation, the tumor weight of vehicle group, positive group and FS group were 0.88 g, 1.02 g and 0.06 respectively, the tumor weight was shown in table 4.

Body Weight

Loss of body weight or a sign of toxicity was not found in FS-treated groups.

The effect on body weight was shown in table 5.

Discussion

EL-7404 peritoneal Implantation hepatic cancer model was used to evaluate the anti-cancer efficacy of FS at 2 ml/mouse. On day 21 after implantation, all mice in vehicle group and positive group exhibited palpable tumors, while no mice in FS group exhibited any palpable tumors. On day 28 after implantation, the tumor in vehicle group reached 0.88 g, while FS group was only 0.06 g. On day 52 after implantation, Five mice treated with FS did not exhibit any palpable tumor. The results showed that FS had inhibition on tumor growth.

No body weight loss and toxicity were found in FS-treated groups, which showed FS had no side effect.

Tables

TABLE 3
Ratios of palpable tumors observed in different groups

| | Days after surgery | | | | | | | | | |
|---|---|---|---|---|---|---|---|---|---|---|
| Group | 21 | 24 | 28 | 31 | 35 | 38 | 42 | 45 | 49 | 52 |
| Vehicle | 3/3 | 3/3 | 3/3 | sacrificed | sacrificed | sacrificed | sacrificed | sacrificed | sacrificed | sacrificed |
| Positive | 3/3 | 3/3 | 3/3 | sacrificed | sacrificed | sacrificed | sacrificed | sacrificed | sacrificed | sacrificed |
| FS (2 ml) + | 0/3 | 0/3 | 0/3 | 0/3 | 0/3 | 0/3 | 0/3 | 0/3 | 0/3 | 0/3 |
| sorafenib | | | | | | | | | | |
| FS (2 ml) | 0/3 | 0/3 | 1/3 | sacrificed | sacrificed | sacrificed | sacrificed | sacrificed | sacrificed | sacrificed |
| FS (2 ml) | 0/3 | 0/3 | 0/3 | 0/3 | 0/3 | 1/3 | 1/3 | 1/3 | 1/3 | 1/3 |
| FS (2 ml) + | 0/3 | 0/3 | 0/3 | 1/3 | 2/3 | 2/3 | 2/3 | 2/3 | 2/3 | 2/3 |
| oral | | | | | | | | | | |
| application | | | | | | | | | | |

TABLE 4
The summary of FS weight and tumor weight

| | Pre- | | | Days | | |
|---|---|---|---|---|---|---|
| | test | | | after | FS Weight | Tumor Weight |
| Group | Mice # | Mice # | status | implantation | (g) | (g) |
| Vehicle | 1 | 1-1 | sacrificed | 28 | N/A | 0.87 |
| | 2 | 1-2 | sacrificed | 28 | N/A | 0.68 |
| | 3 | 1-3 | sacrificed | 28 | N/A | 1.09 |
| Positive | 4 | 2-1 | sacrificed | 28 | N/A | 3.56 |
| | 5 | 2-2 | sacrificed | 28 | N/A | 1.15 |
| | 6 | 2-3 | sacrificed | 28 | N/A | 1.35 |
| FS (2 ml) + | 7 | 3-1 | died | 22 | 1.25 | 0.00 |
| sorafenib | 8 | 3-2 | sacrificed | 54 | 1.22 | 0.00 |
| | 9 | 3-3 | sacrificed | 54 | 0.92 | 0.00 |
| FS (2 ml) | 10 | 4-1 | sacrificed | 28 | 1.06 | 0.10 |
| | 11 | 4-2 | sacrificed | 28 | 0.72 | 0.00 |
| | 12 | 4-3 | sacrificed | 28 | 0.78 | 0.09 |
| FS (2 ml) | 13 | 5-1 | sacrificed | 54 | 0.81 | 0.75 |
| | 14 | 5-2 | sacrificed | 54 | 0.80 | 0.00 |
| | 15 | 5-3 | sacrificed | 54 | 0.81 | 0.00 |
| FS (2 ml) + | 16 | 6-1 | sacrificed | 54 | 1.08 | 2.16 |
| oral application | 18 | 6-3 | sacrificed | 54 | 0.95 | 0.00 |
| | 19 | 6-2 | died | 51 | 0.91 | 3.03 |

1) Ovarian Cancer Survival Rate in Mice

Ovarian cancer study has been conducted at Wuxi AppTec under Dr. Yong Cang and the study is still ongoing as of Aug. 4, 2013. However in order to show the comparison mean days of the death of the mice in each group to compare with the positive drug group which has 76.8 days. The vehicle with 128.5 days and two AFOD RAAS 108 from A1AT from fraction IV has 165.2 days. And AFCC RAAS 2 from fraction IV, has 162.3 days.

Mean Survival Time (Days)

| | Mean (days) | std error |
|---|---|---|
| AFOD RAAS 1 | 115.8 | 12.7 |
| AFOD RAAS 104 | 114.5 | 22.0 |
| AFOD RAAS 108 | 165.2 | 8.5 |
| AFOD RAAS 109/121 | 125.3 | 10.5 |
| AFOD RAAS 110 | 131.8 | 5.9 |
| AFOD RAAS 113 | 110.7 | 9.2 |
| AFOD RAAS 114 | 97.8 | 10.0 |
| AFOD RAAS 120 | 84.8 | 5.6 |
| AFCC RAAS 1 | 97.0 | 26.9 |
| AFCC RAAS 2 | 162.3 | 11.6 |
| positive | 76.8 | 14.2 |
| vehicle | 128.5 | 14.7 |

Survival curve for AFOD RAAS 1, AFOD RAAS 104, AFOD RAAS 108, AFOD RAAS 109/121. AFOD RAAS 110, AFOD RAAS 113, AFOD RAAS 114, AFOD RAAS 120 AND AFCC RAAS 1, AFCC RAAS 2, POSITIVE AND VEHICLE.

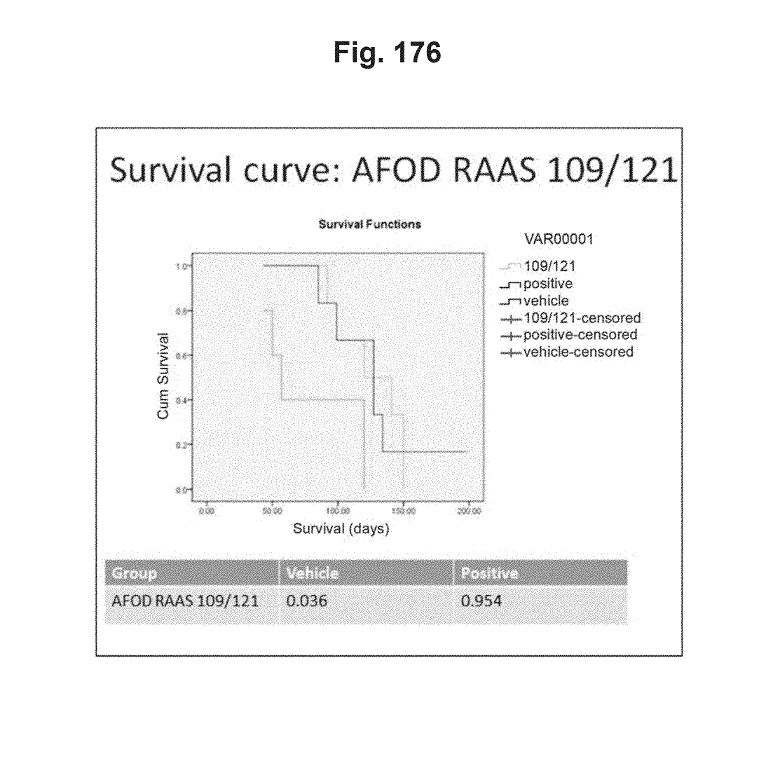

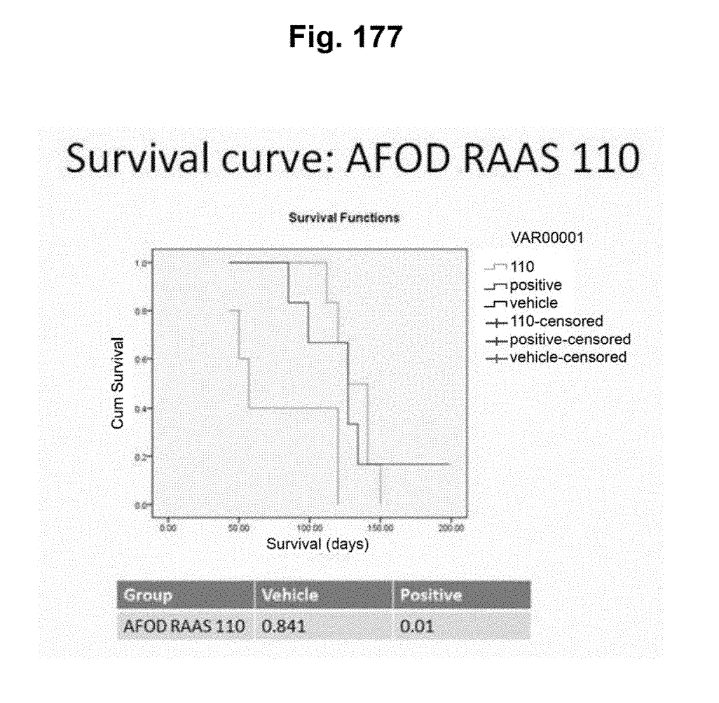

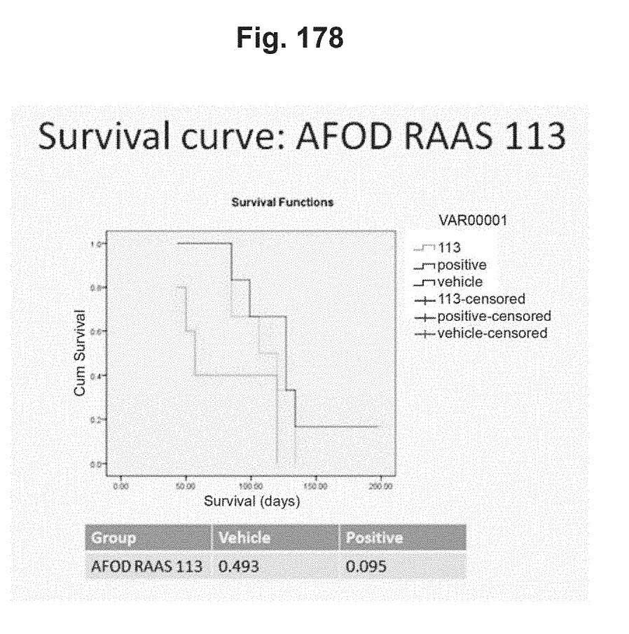

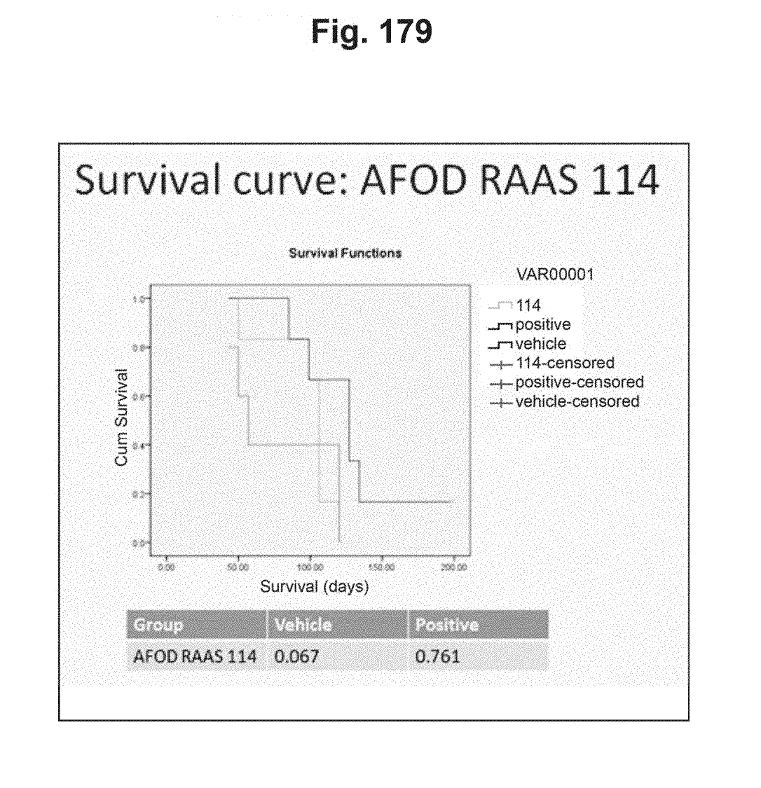

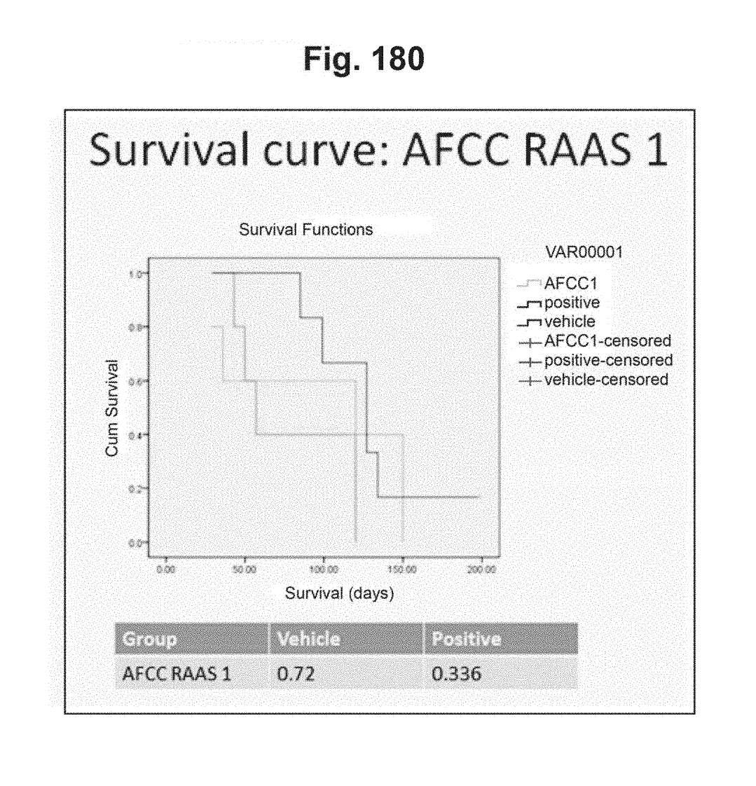

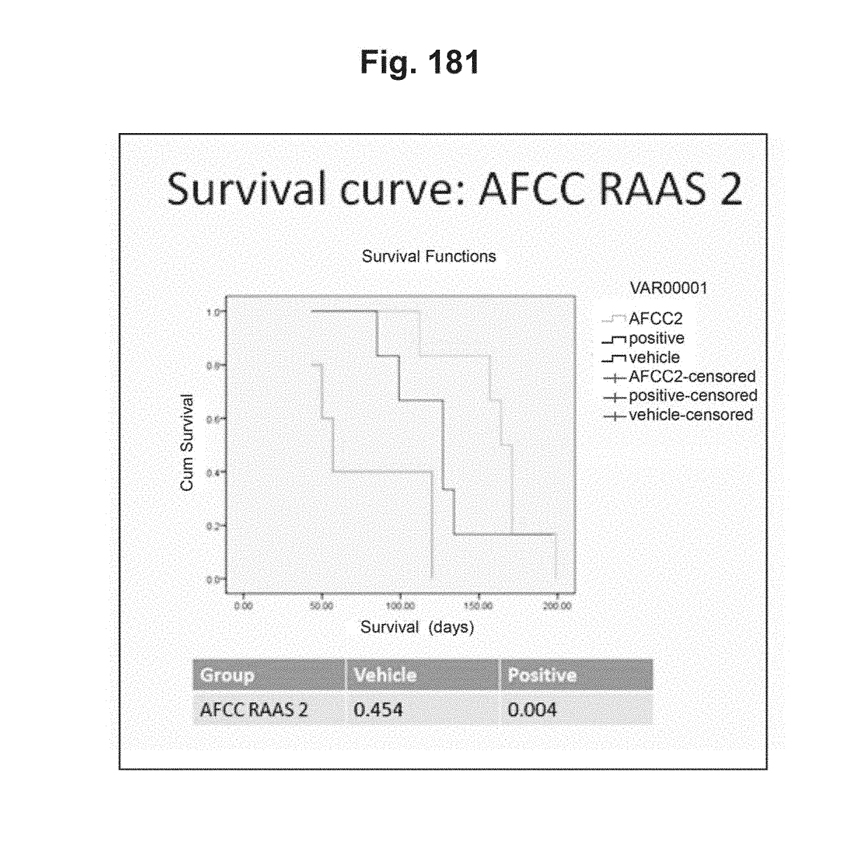

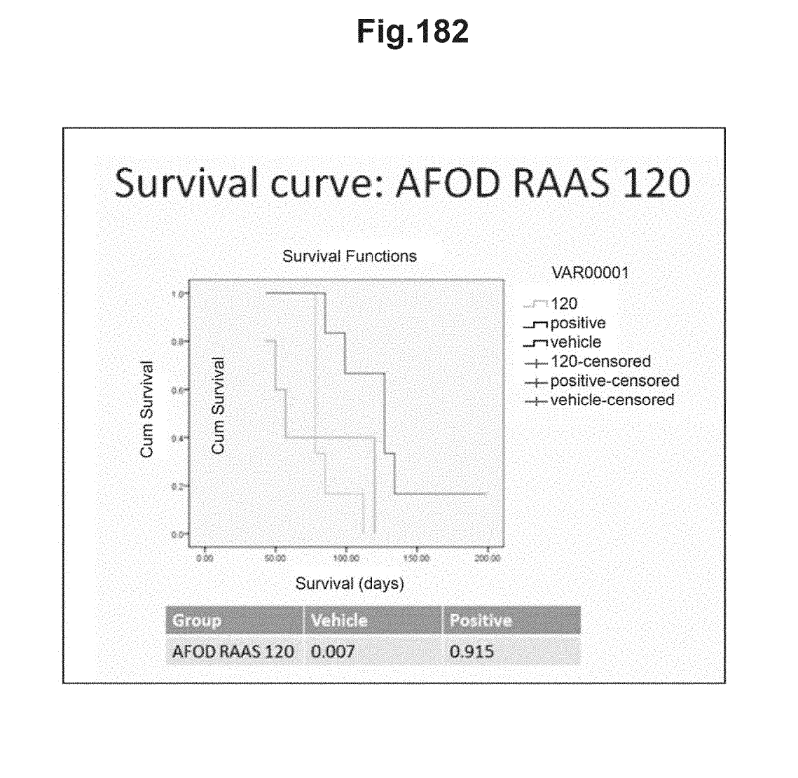

FIGS. 173, 174, 175, 176, 177, 178, 179, 180, 181 and 182

Bioluminescent images of RAAS-20120628-SK-OV-3-luc for all groups.

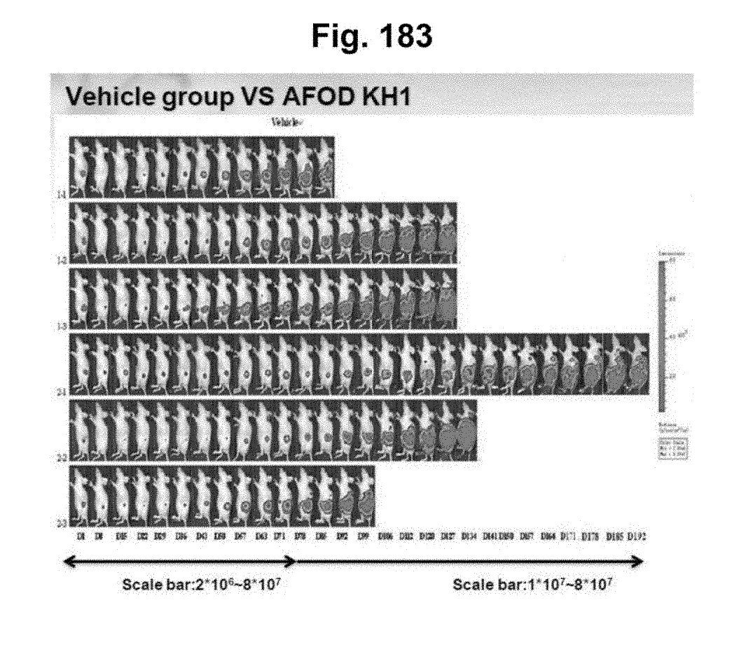

FIGS. 183—Vehicle group

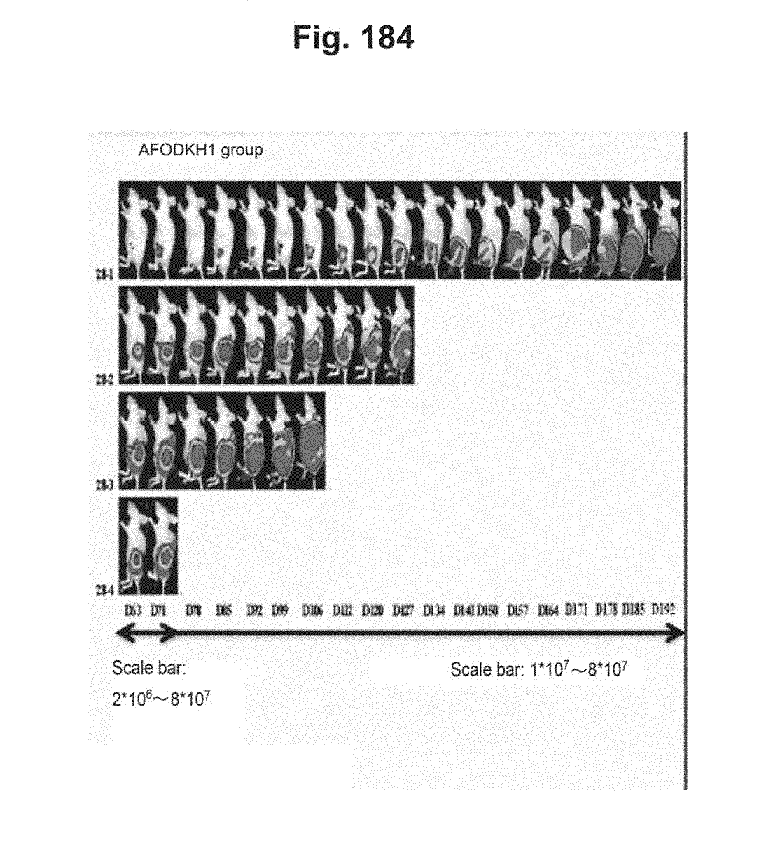

FIGS. 184—AFOD KH1 (Kieu Hoang AFCC TM Wine). At day 63 after ovarian cancer has started an experiment of Kieu Hoang AFCC TM by oral application of four mice left from the entire group. That means that by day 63 ovarian cancer has already developed. In the beginning we forced the mice to drink without water, therefore the mice died in early stage. Among the four mice, one lasted to 192 days like those mice in AFOD RAAS 8 and AFCC RAAS 2 totaling 6 mice.

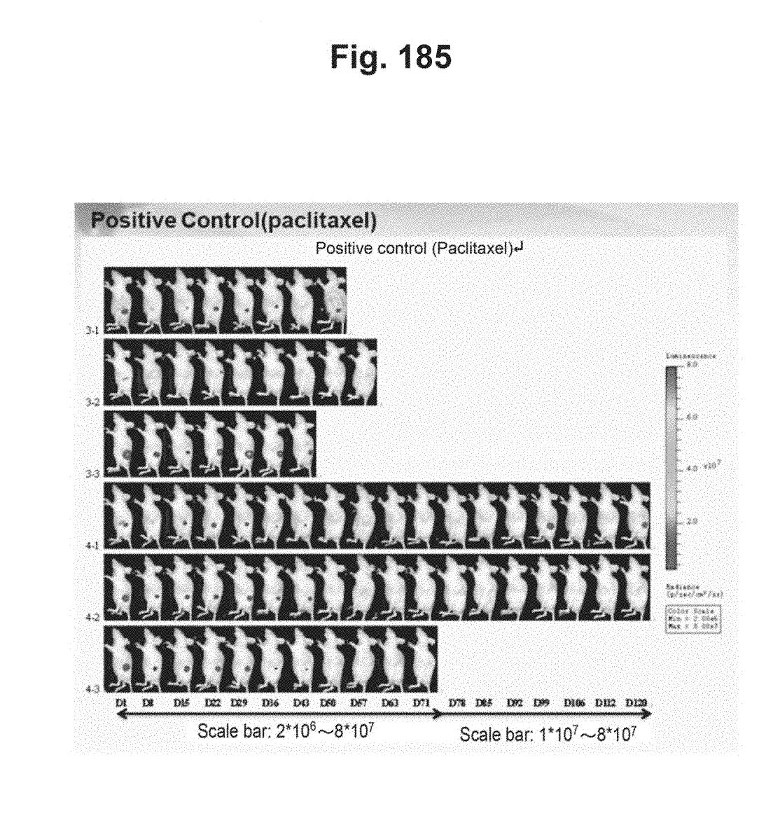

FIG. 185—Positive control

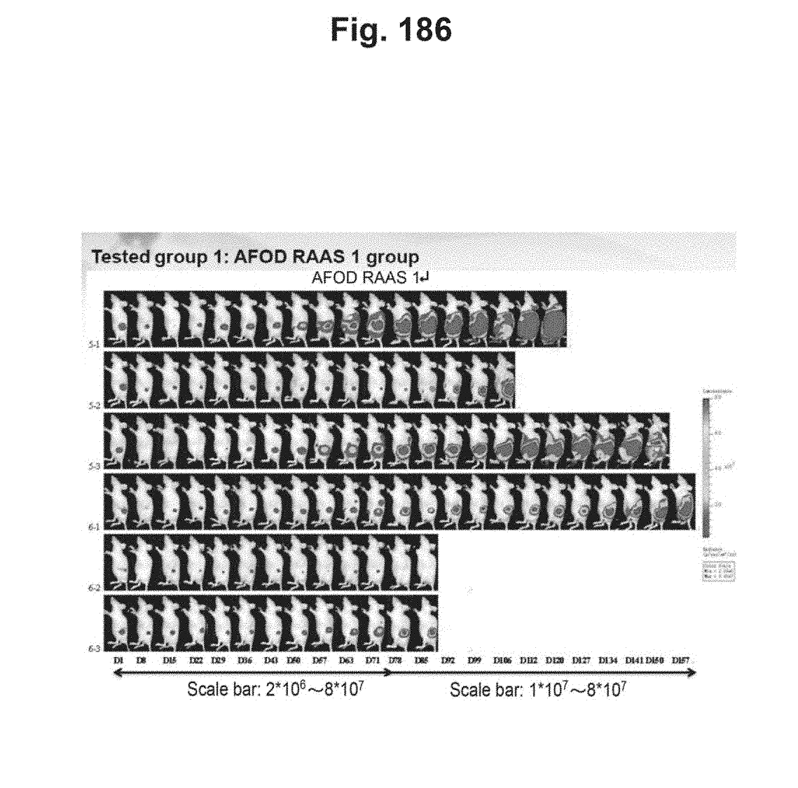

FIG. 186—Tested group 1: AFOD RAAS 1 group

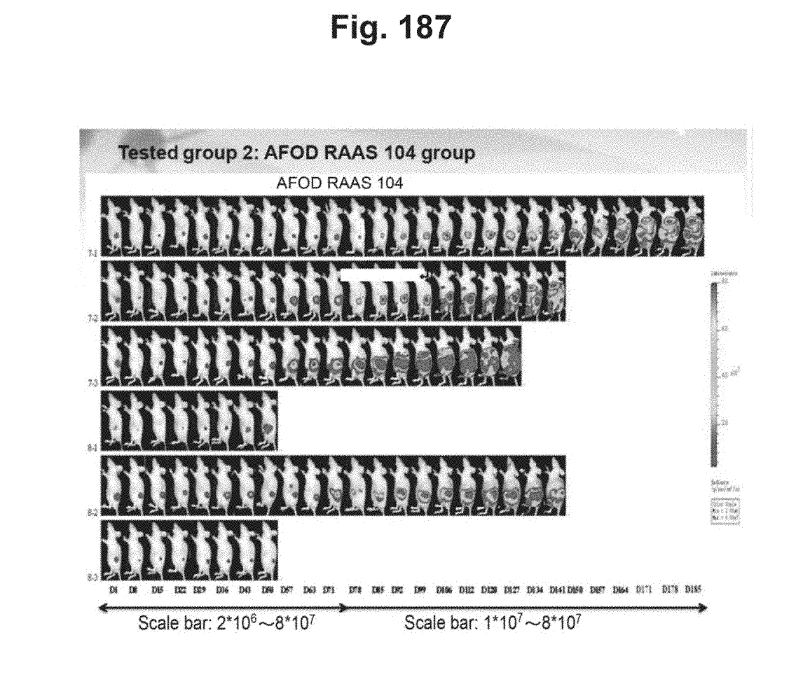

FIG. 187—Tested group 2: AFOD RAAS 104

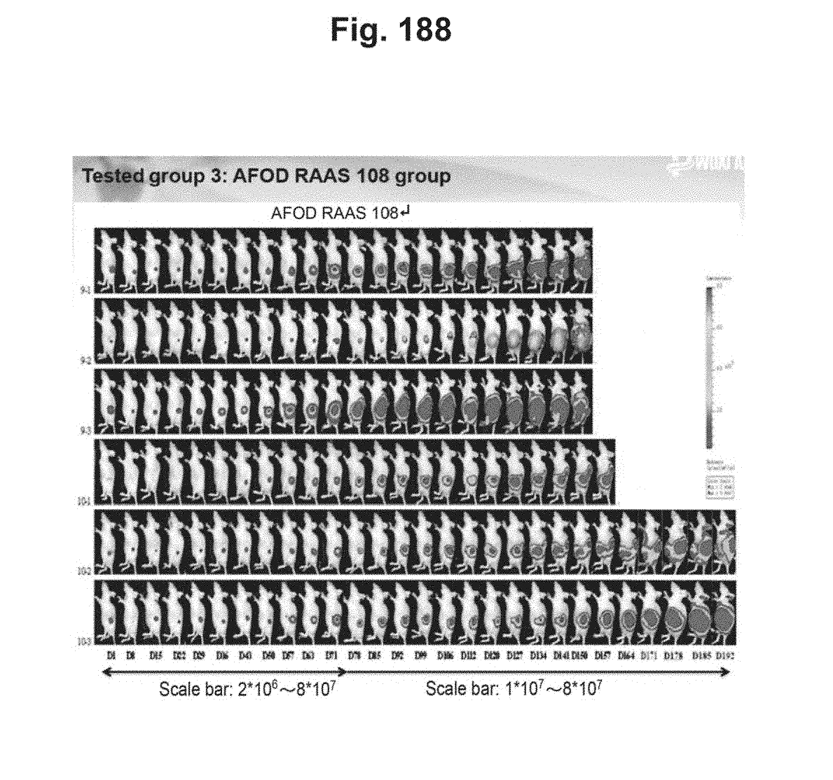

FIG. 188—Tested group 3: AFOD RAAS 108

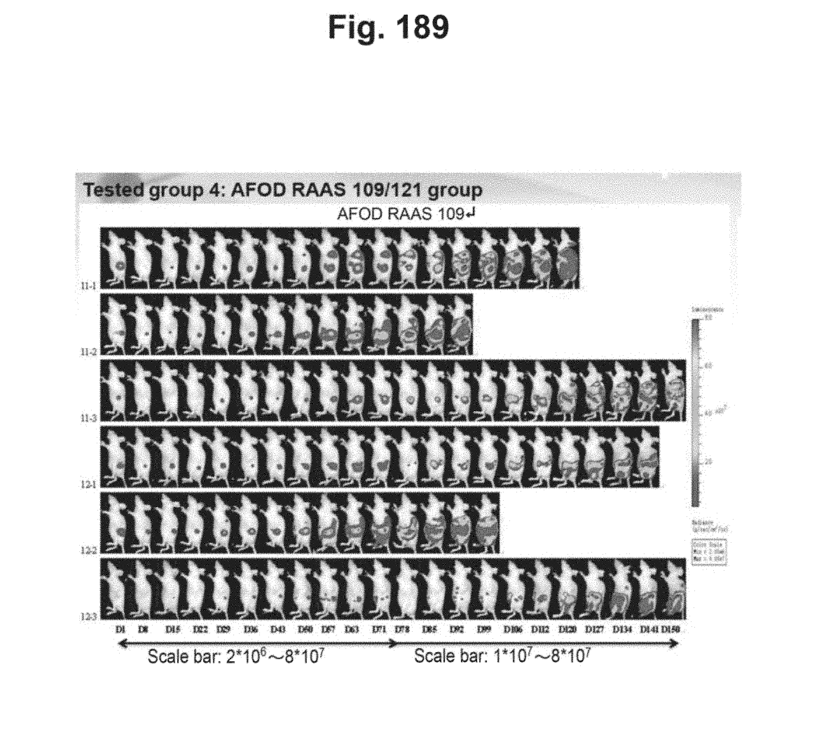

FIG. 189—Tested group 4: AFOD RAAS 109/121

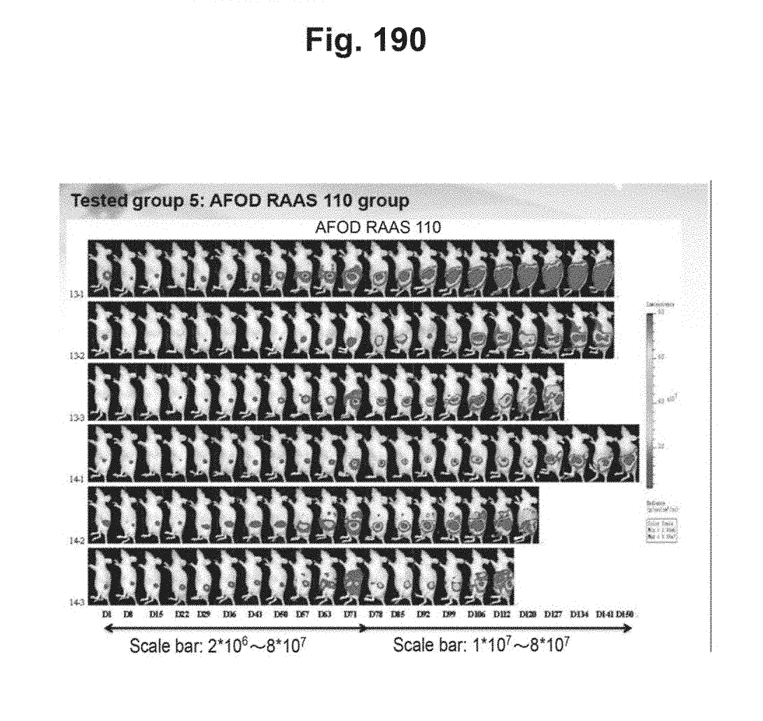

FIG. 190—Tested group 5: AFOD RAAS 110

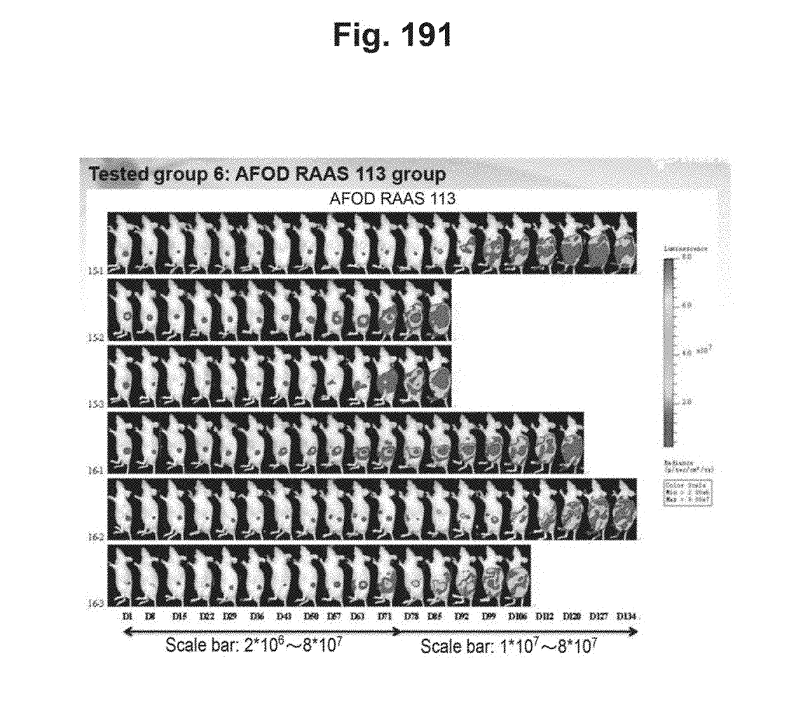

FIG. 191—Tested group 6: AFOD RAAS 113

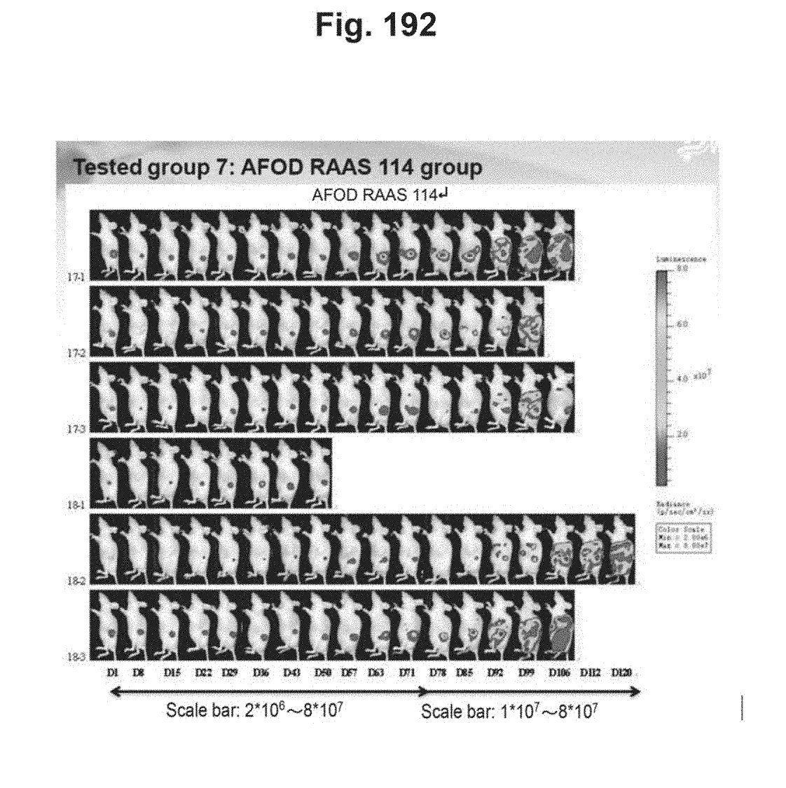

FIG. 192—Tested group 7: AFOD RAAS 114

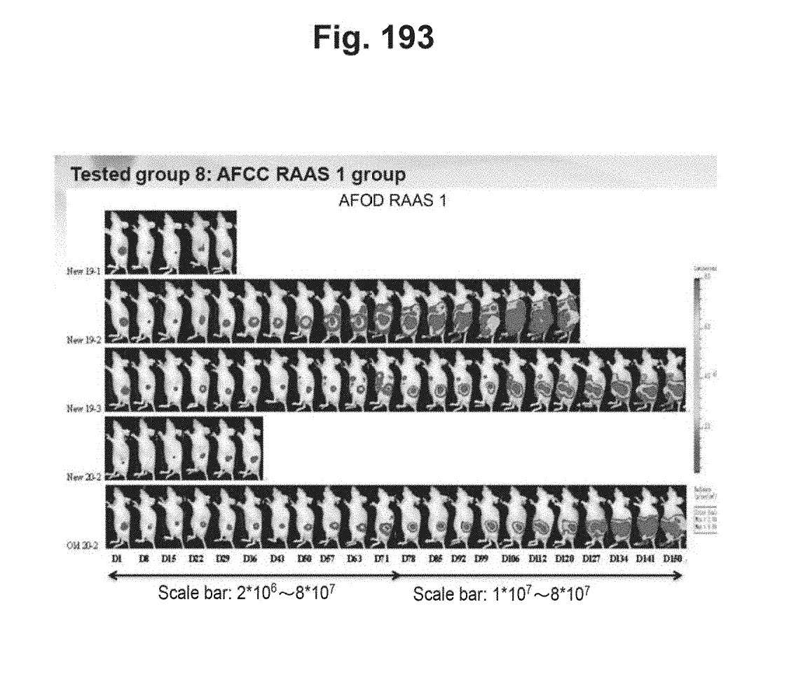

FIG. 193—Tested group8: AFCC RAAS 1

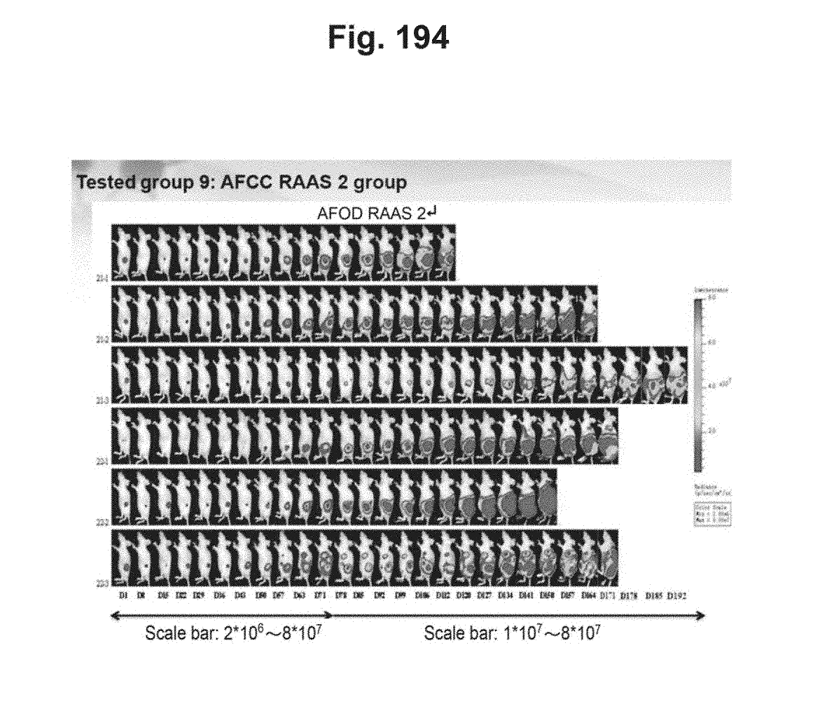

FIG. 194—Tested group 9: AFCC RAAS 2

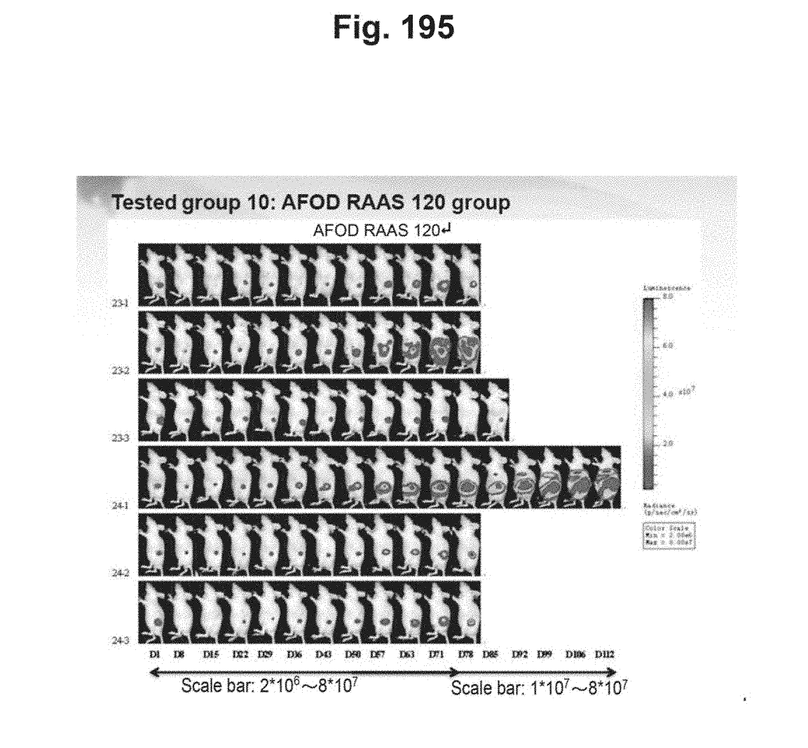

FIG. 195—Tested group 10: AFOD RAAS 120

Characterization of Lymphoid Tissues and Peripheral Blood in Nude Mouse Treated with and without AFCC

Executive Summary

The purpose of this study was to investigate the effect of AFCC on curing tumor through characterizing distinct cell lineage in lymphoid tissues and peripheral blood in nude mouse treated with and without AFCC. Distinct cell lineage was differentiated by cell surface marker proteins. T cells, B cells, activated B cells, myeloid dendritic cell (mDC), plasmacytoid dendritic cell (pDC), granulocytes, and monocytes/macrophages were characterized.

In spleen and lymph nodes except in peripheral blood, AFCC treatment resulted in increased CD3⁺T cell population compared with that in nude mouse with tumor. In spleen, lymph nodes, and peripheral blood, with AFCC treatment, B cell population together with activated B cells also increased compared with those in nude mouse with tumor. In spite of the increased cell number of B cells and T cells after AFCC treatment, granulocytes decreased. Macrophages were found to decrease after AFCC treatment in peripheral blood and spleen but not in draining lymph nodes. mDC and pDC percentages were not greatly affected in nude mouse in the presence of AFCC.

List of Abbreviations

| | |
|---|---|
| FACS | Flow Cytometry |
| mDC | Myeloid dendritic cell |
| pDC | Plasmacytoid dendritic cell |

Materials and Methods

Materials

Reagents

FITC, Rat Anti-Mouse CD4, BD, Cat: 557307

FITC, Rat Anti-MouseCD3 molecular complex, BD, Cat: 561798

PerCP-Cy5.5, Rat Anti-Mouse CD4, BD, Cat: 550954

PE, Rat Anti-Mouse B220/CD45R, BD, Cat: 553089

APC, Rat Anti-MouseCD11b, BD, Cat: 553312

APC, Ar Ham Anti-MouseCD11c, BD, Cat: 550261

PE, Rat Anti-MouseGR-1(Ly-6G and Ly-6C), BD, Cat: 553128

Purified, Rat Anti-MouseFc blocker CD16/32, BD, Cat: 553141

APC, Ar Ham Rat Anti-MouseCD69, BD, Cat: 560689

7-AAD, BD. Cat: 559925

ACK Lysing buffer, Invitrogen, Cat: A10492-01

PBS, Dycent Biotech (Shanghai) CO., Ltd. Cat: BJ141.

FBS, Invitrogen Gibco, Cat: 10099141

Materials

Cell strainer (70 μm), BD, Cat: 352350

BD Falcon tubes (12×75 mm, 5 ml), BD, Cat: 352054

Equipments

Vi-CELL Cell Viability Analyzer, Beckman Coulter, Cat: 731050

FACSCalibur flow cytometer, BD, Cat: TY1218

Methods

Cell Isolation and Staining

Peripheral blood was collected through cardiac puncture. After removing red blood cells with lysis buffer followed by two rounds of washing using 1×PBS, mononuclear cells (monocytes, macrophages, dendritic cells, and lymphocytes) and granulocytes were obtained. Spleen and lymph nodes cell suspension were also obtained after filtering through 70 μm cell strainer. Cell viability and number were analyzed by Vi-CELL Cell Viability Analyzer. Cell surface labeling was performed after that. Blocked with Fc blocker CD16/CD32 at 4° C. for 15 min, cells were centrifuged and resuspended in staining buffer (0.08% NaN3/PBS+1% FBS). Fluorescent-conjugated antibodies were then added into the suspension at the indicated dilution according to the antibody usage protocol from the company. After 30 min incubation at 4° C. for 30 min in the dark, cells were washed twice with 0.08% NaN3/PBS (200 μl per sample), and resuspended with 400 μl 0.08% NaN₃/PBS in BD Falcon tubes (12×75 mm, 5 ml) followed by FACS analysis.

Data Analysis

FACS data were analyzed by flowjo software.

Study Summary

Study Initiation Date and Completion Date

The study was initiated and finished on Apr. 13, 2012.

Study Purpose

The purpose of this study was to investigate the effect of AFCC on curing tumor through characterizing distinct cell lineage in lymphoid tissues and peripheral blood in nude mouse treated with and without AFCC.

Study Results

Mice Information

All the mice were transferred from oncology team from Wuxi Apptec. FIG. 1 and FIG. 2 contained the treatment and age information of the mice.

1: Nude mice with tumor: nude mice grafted with MDA-MB-231-Luc tumor cells as vehicle for the study.

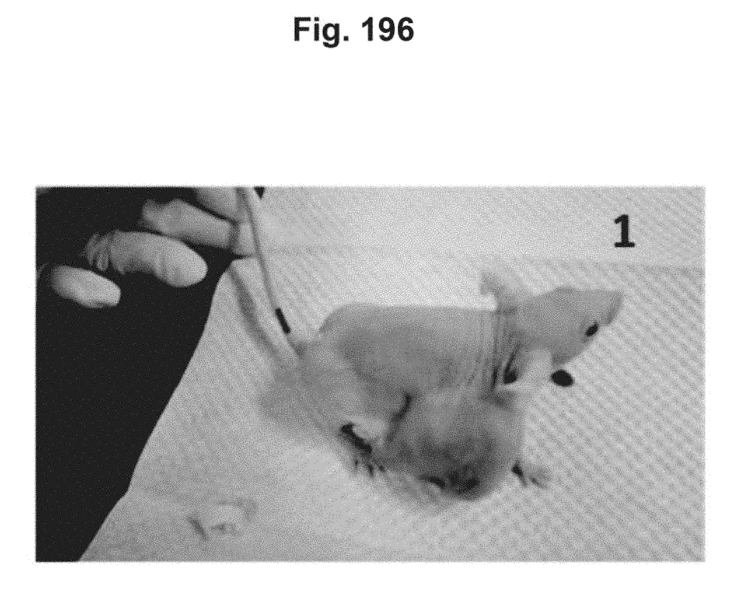

FIG. 196

10 nude mice from group 2-5 which have been implanted with tumor cells from the 2-5 mice positive control group using Docetaxel in another study done at another CRO lab.

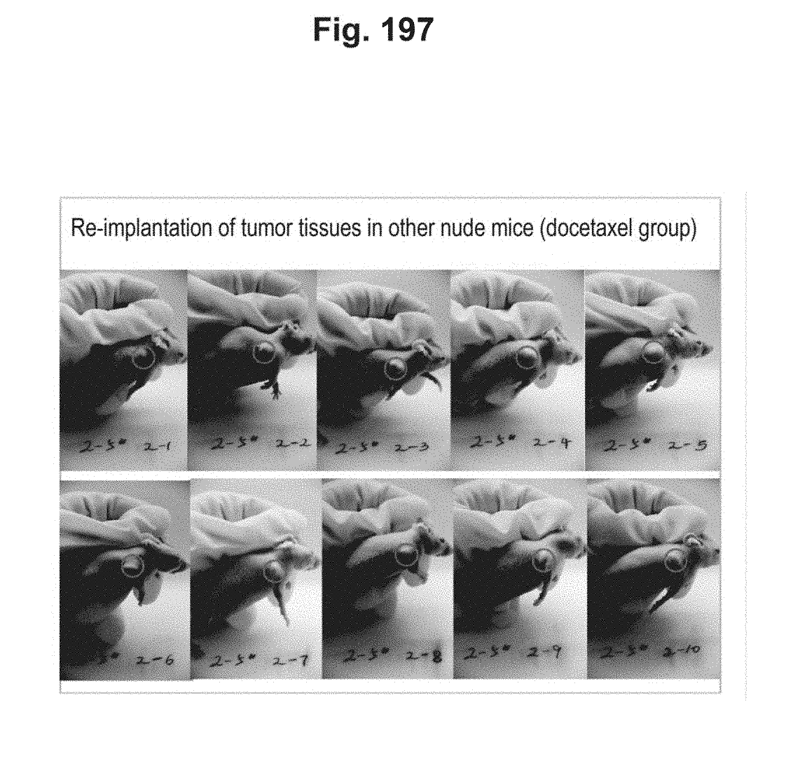

FIG. 197

3: One of the 10 nude mice with MDA-MB-231-Luc tumor cells transferred from 2-5 positive control group using Docetaxel and it is used as positive control for the re-implantation study,

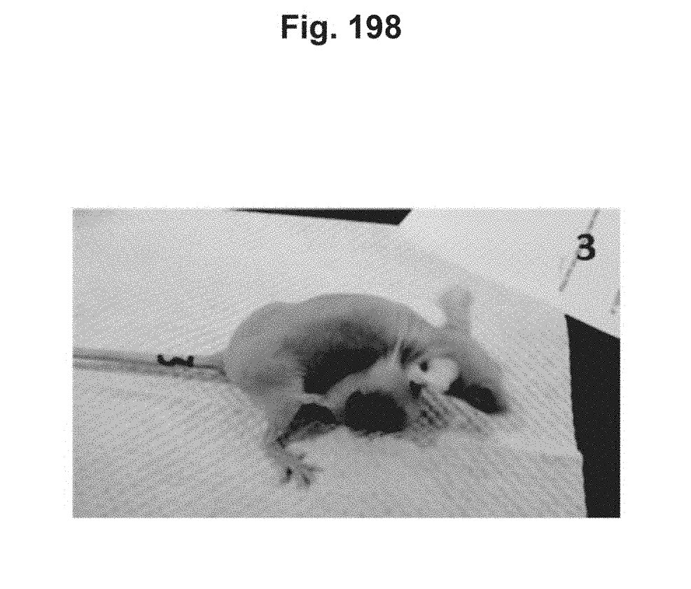

FIG. 198

Graph showing the tumor volume of Mice #6-10 from the study done from Jul. until Nov. 11, 2011 when the dead body of mouse #6-10 was removed from one CRO lab to another one for further study.

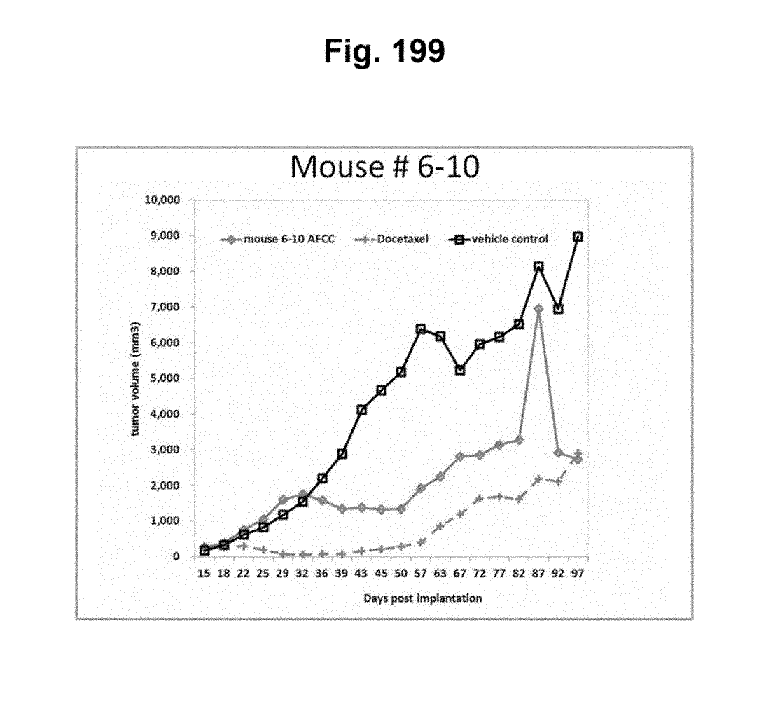

FIG. 199

Mouse #6-10 taken from Aug. 23, 2011 to Nov. 3, 2011 showing the growth of the tumor which had been detached from the body was under recovery from breast cancer using AFCC proteins for treatment.

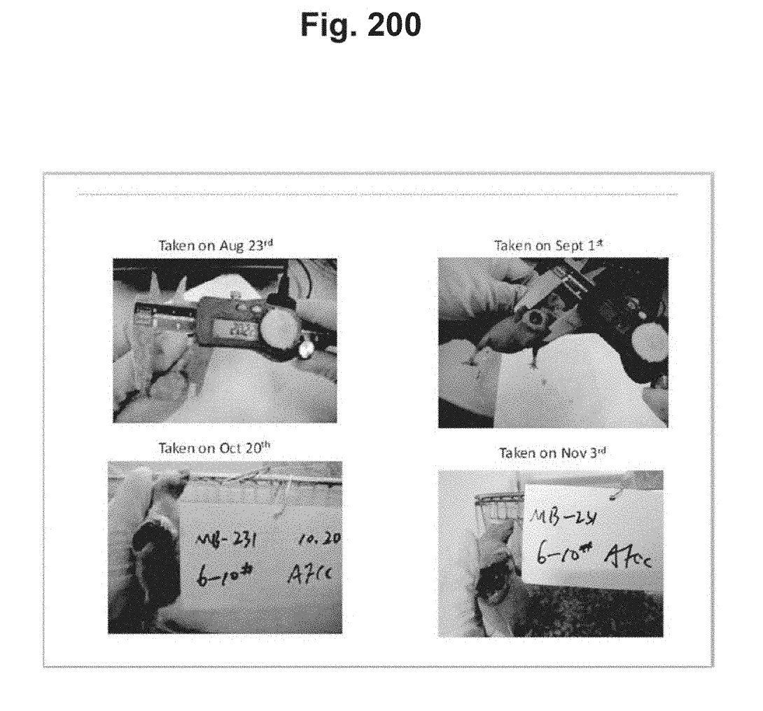

FIG. 200

The tissue from the area of mouse #6-10 where the tumor had been detached was used to implant in the 10 nude mice 66 days after re-implantations show no tumor growth.

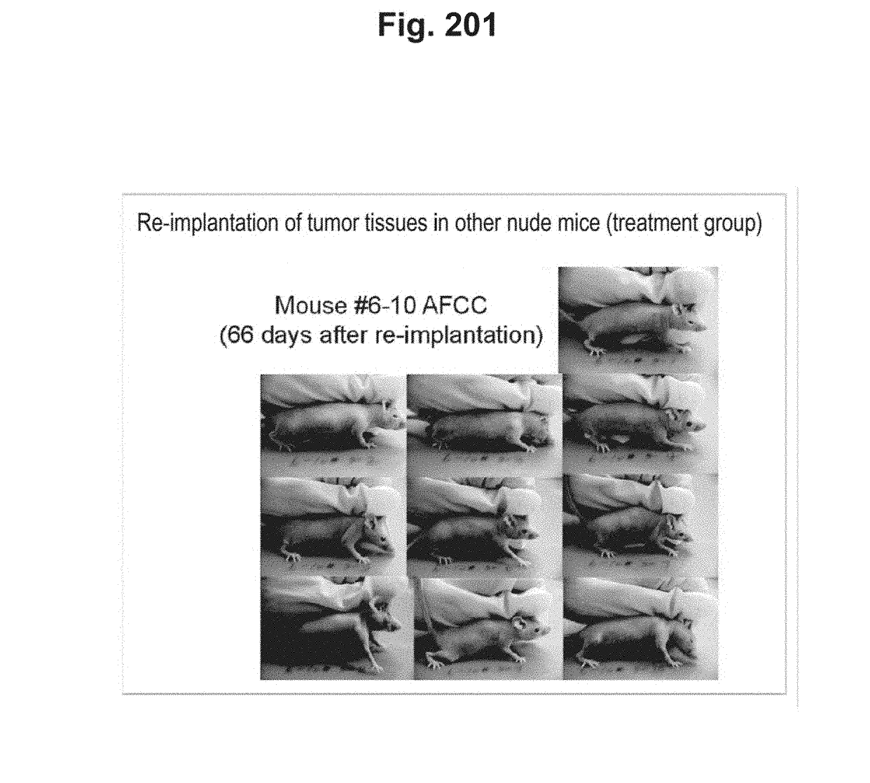

FIG. 201

After 66 days with no growth, then we implanted the cancer tumor for a second time. The growth of the tumor in mice 6-10 which had been treated prior with AFCC at another CRO lab after re-implantation on Nov. 11, 2011.

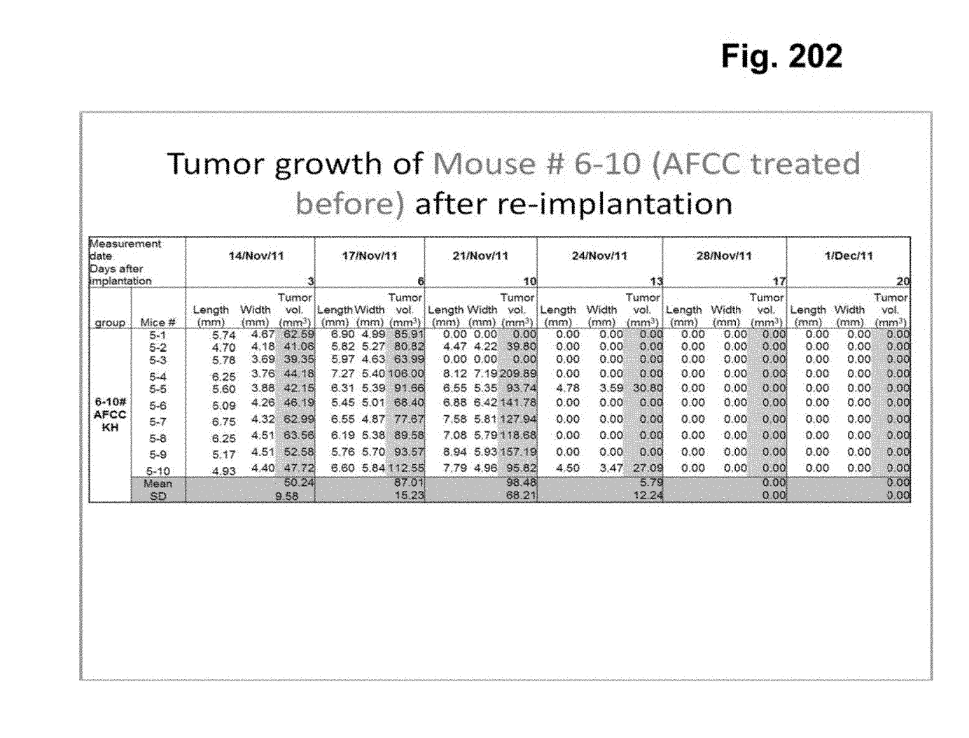

FIG. 202

Graph showing 5 groups of nude mice after tumor volume change after the second re-implantation with the breast tumor cancer, including mice #6-10 and mice #2-10 treated with Docetaxel.

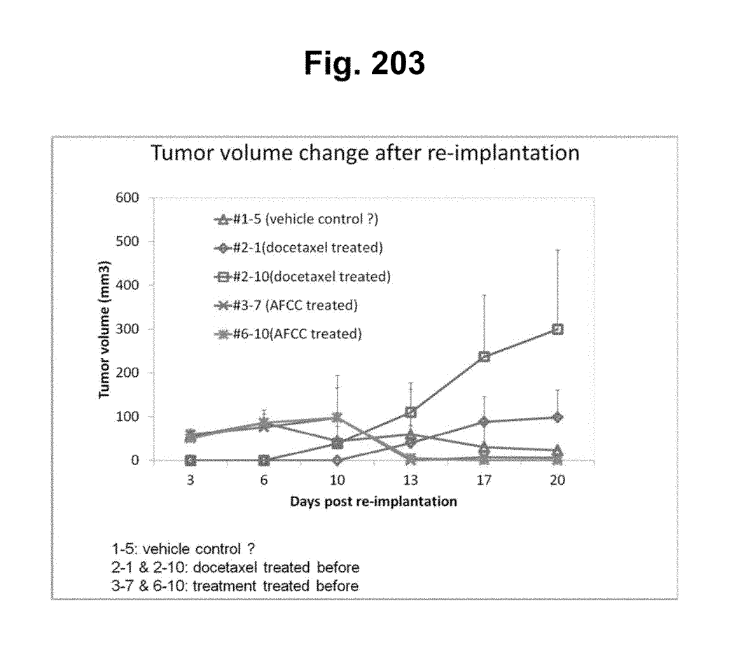

FIG. 203

The picture of the 10 mice in group #6-10 showing mice #5-1 and mice #5-3 growing the tumor after second re-implantation both had been treated with AFCC on Feb. 29, 2012.

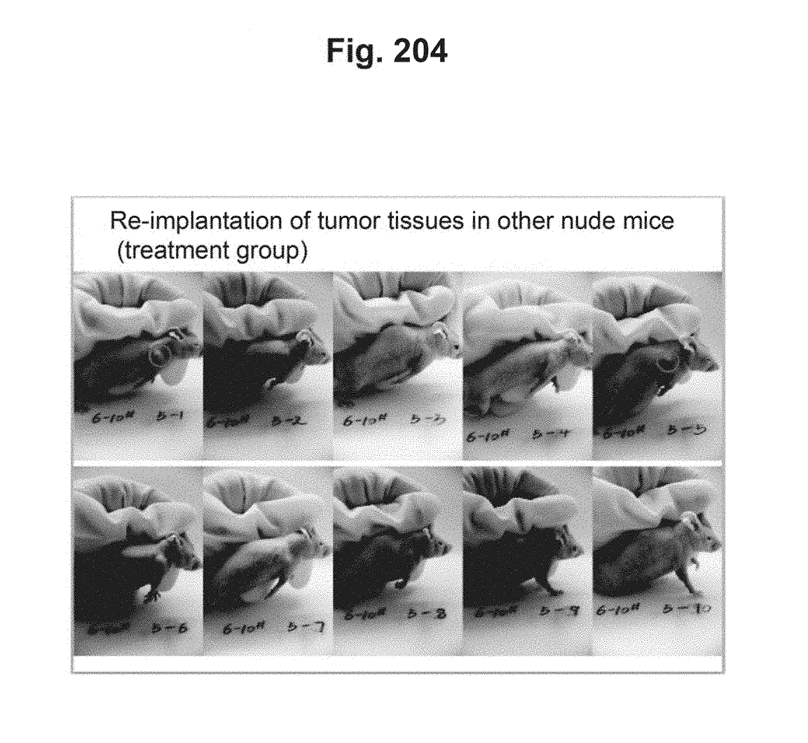

FIG. 204

2: Nude Mice with AFCC Treatment:
Grafted with tumor cells numbered #6-10 starting at Nov. 11, 2011; received with AFCC provided by RAAS though I.V. or I.P. injection from Feb. 29, 2012. In April mice #6-10 with the second re-implantation has been completely recovered due to the AFCC proteins which contain good healthy cells which sent signal to the DNA of the infected mice with breast cancer tumor, to transform the RNA to synthesize good proteins against the breast cancer cell.

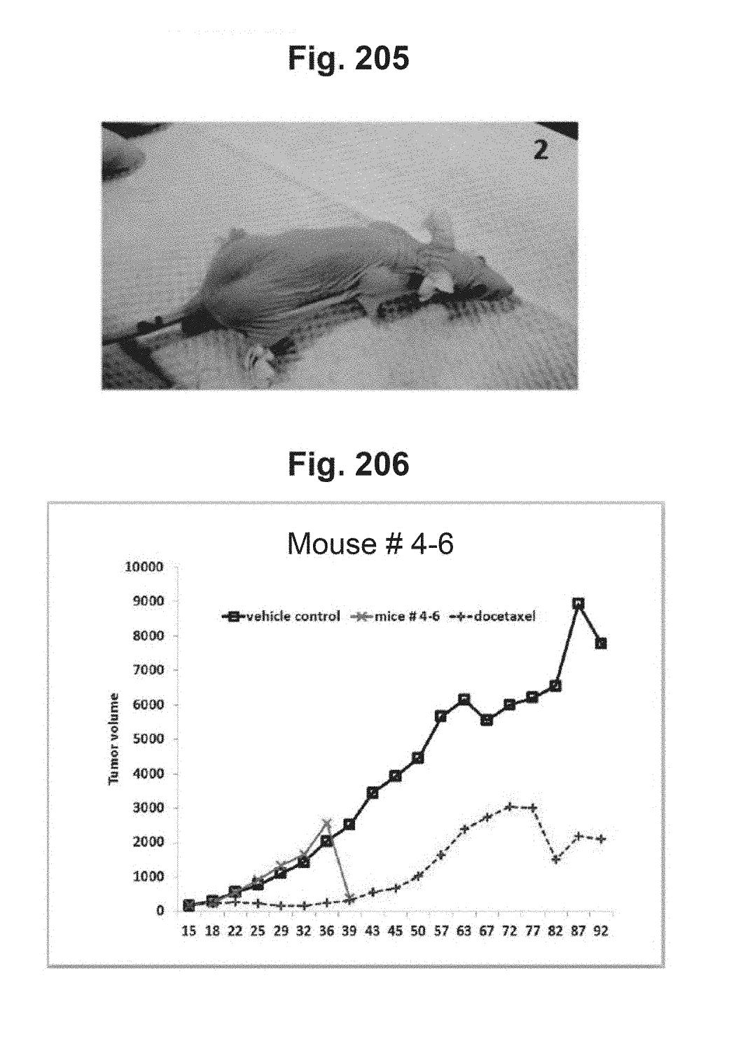

FIG. 205.

Among the groups in the study for breast cancer from mid-Jul. to Nov. 11, 2011 nude mouse #4-6 has shown the quickest recovery period within 24 days. From day 15 when the tumor started to grow to day 39 when the tumor detached from the body.

FIG. 206

Mouse #4-6 grew the tumor on August 23^{rd} and self-detached from the body Sep. 1, 2011.

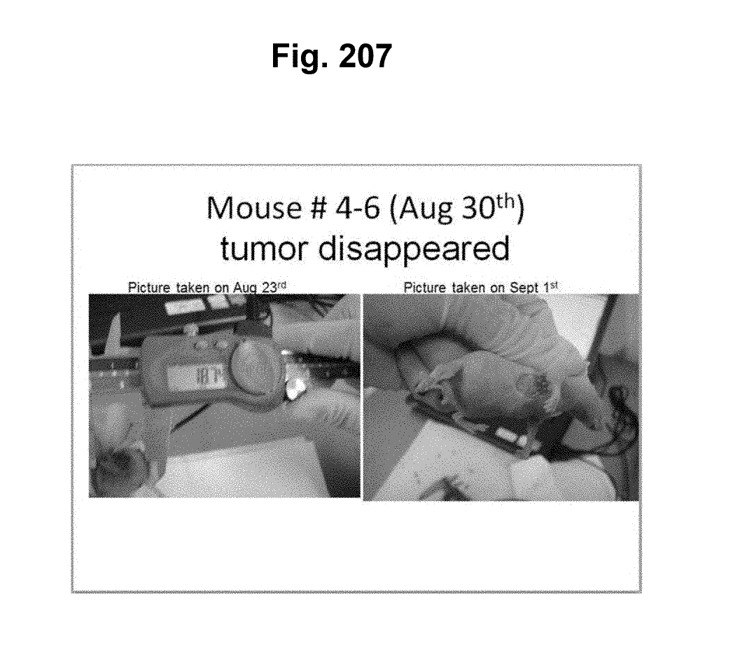

FIG. 207

Mouse #4-6 on October 18^{th} completely recovered from breast cancer due to the AFCC KH protein which contains good healthy cells which sent signal to the DNA of the infected mice with breast cancer tumor, to transform the RNA to synthesize good proteins against the breast cancer cell.

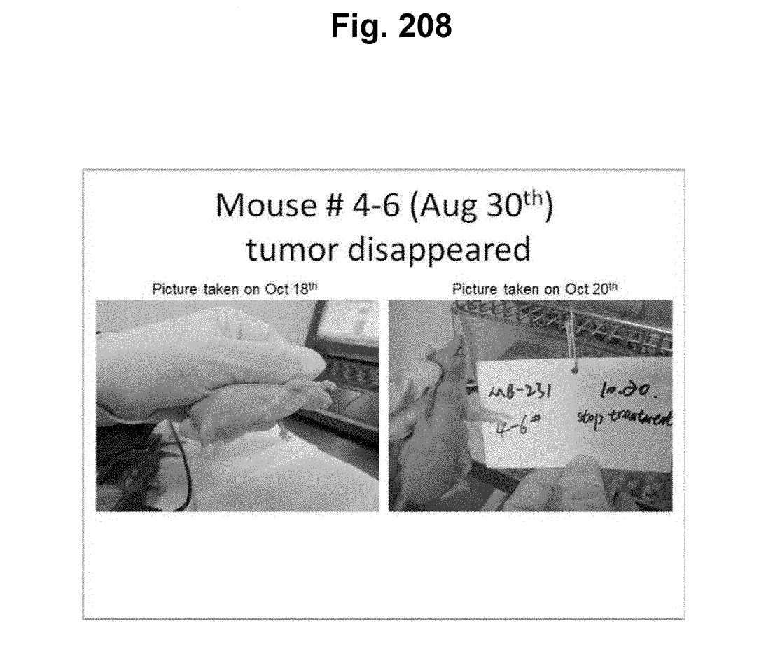

FIG. 208

The 9 mice from the #4-6 group first re-implantation of the tumor which had never grown and one of these mice #4 was used in this study for analysis of the cells.

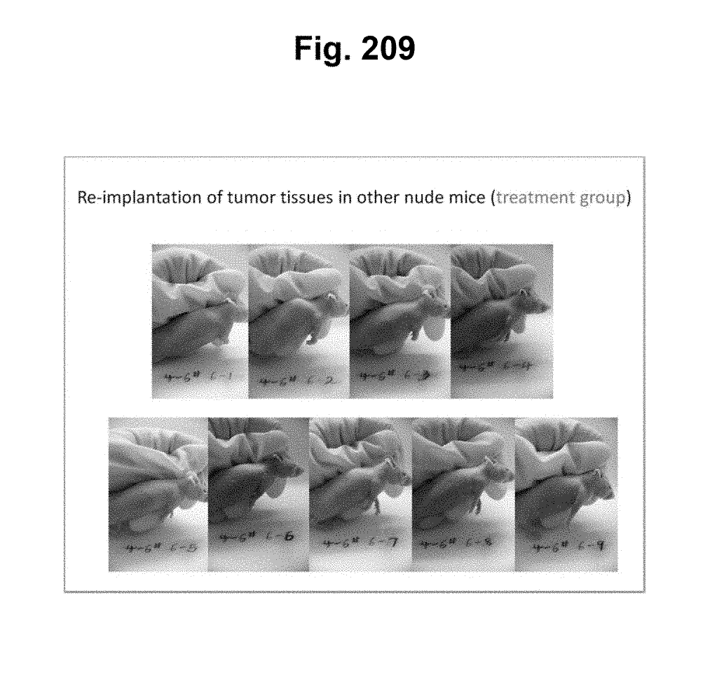

FIG. 209

4: Nude mouse with no tumor: grafted with tumor cells numbered #4-6 starting at Nov. 18, 2011, no further treatment needed due to failure of the tumor growth because good healthy cells from the AFCC treated, which contains good healthy cells which sent signal to the DNA of the infected mice with breast cancer tumor, to transform the RNA to synthesize good proteins against the breast cancer cell.

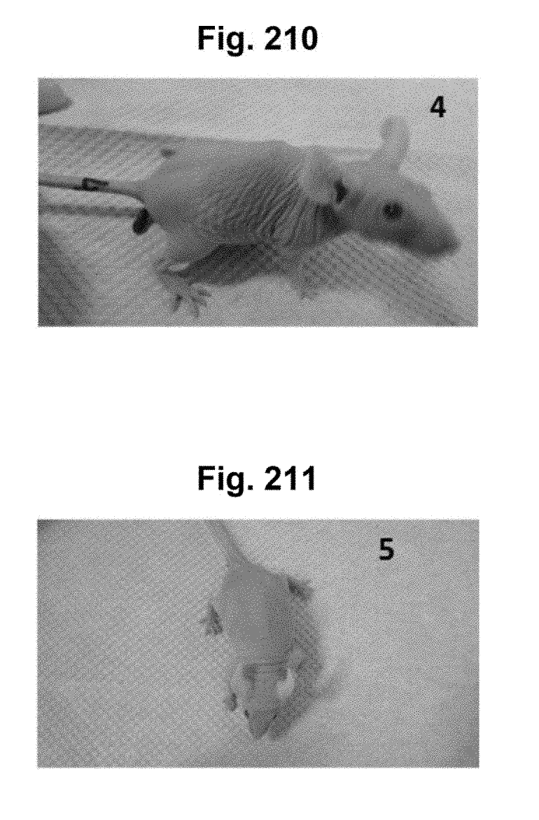

FIG. 210

5: Nude naïve mouse at 8 weeks old was used as a negative normal control to determine the normal nude mice cells.

FIG. 211

6: C57BL/6 mouse at 8 weeks old was used as a negative normal control to determine the normal nude mice cells.

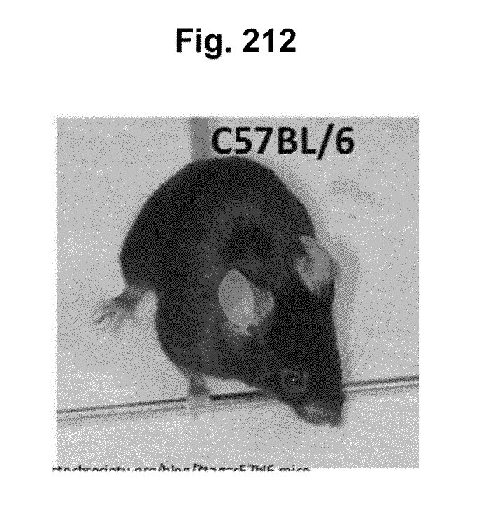

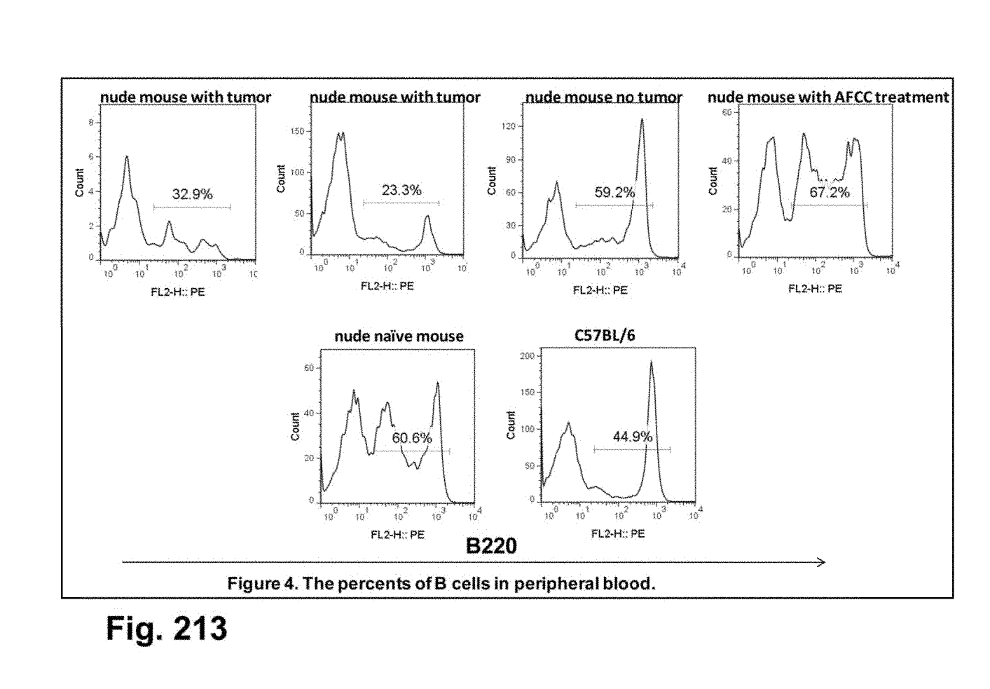

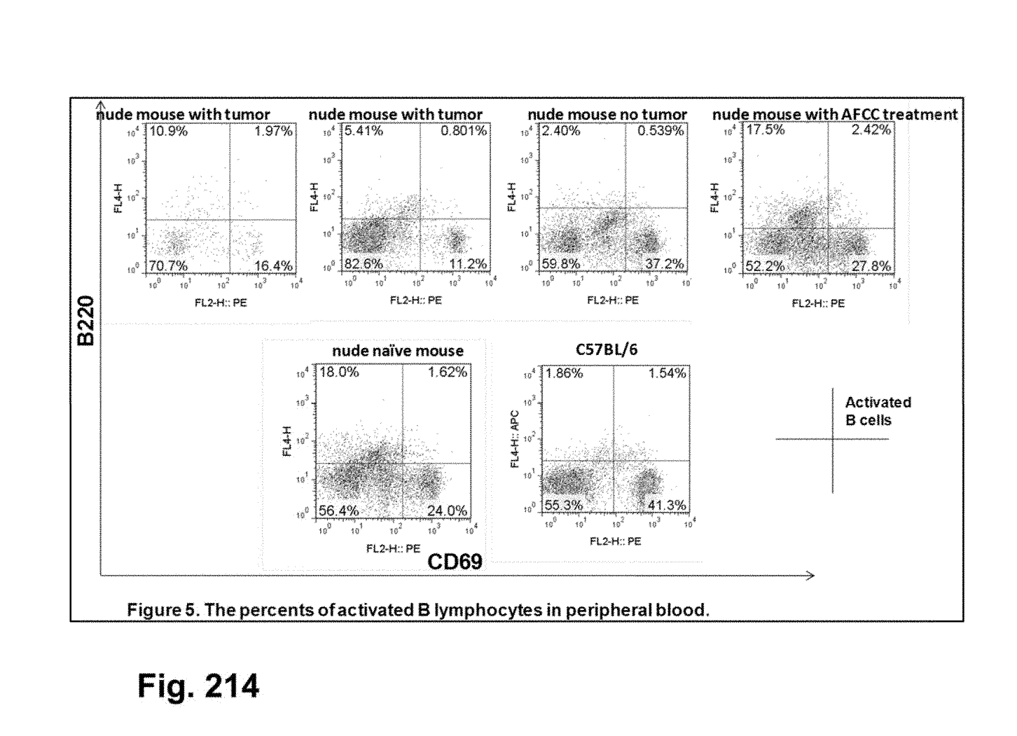

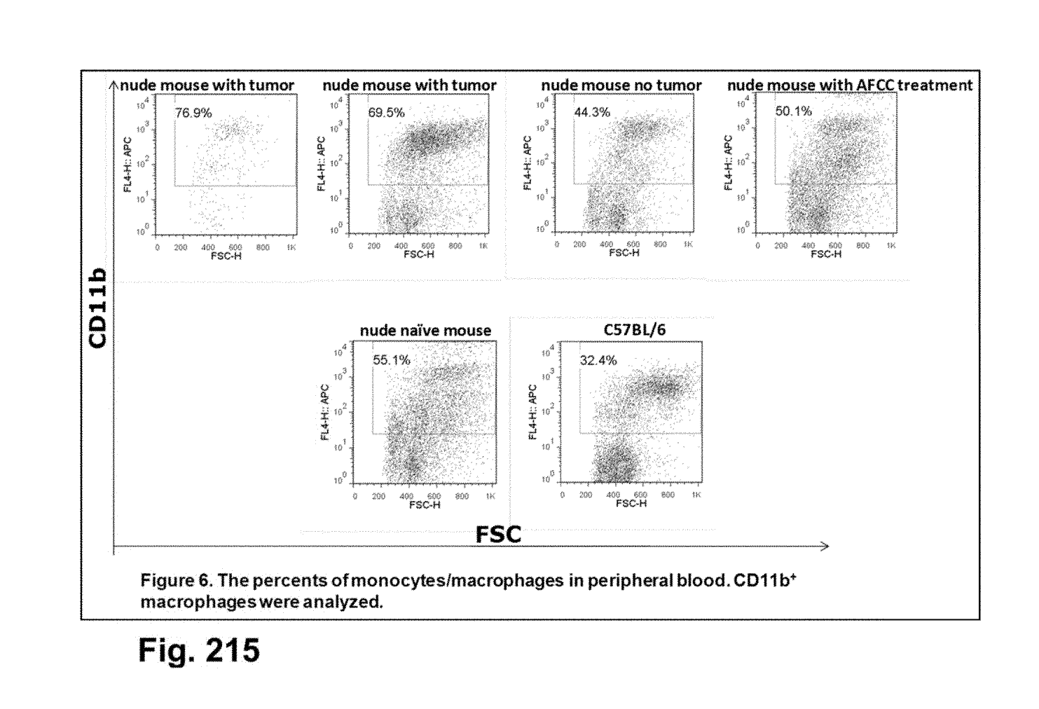

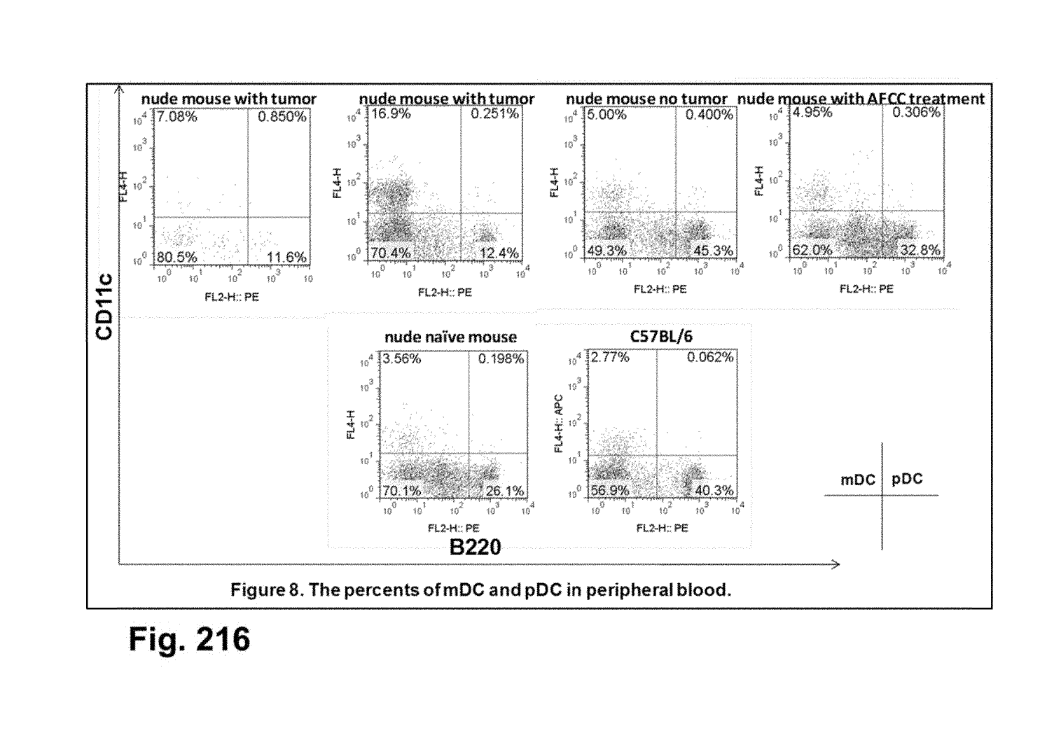

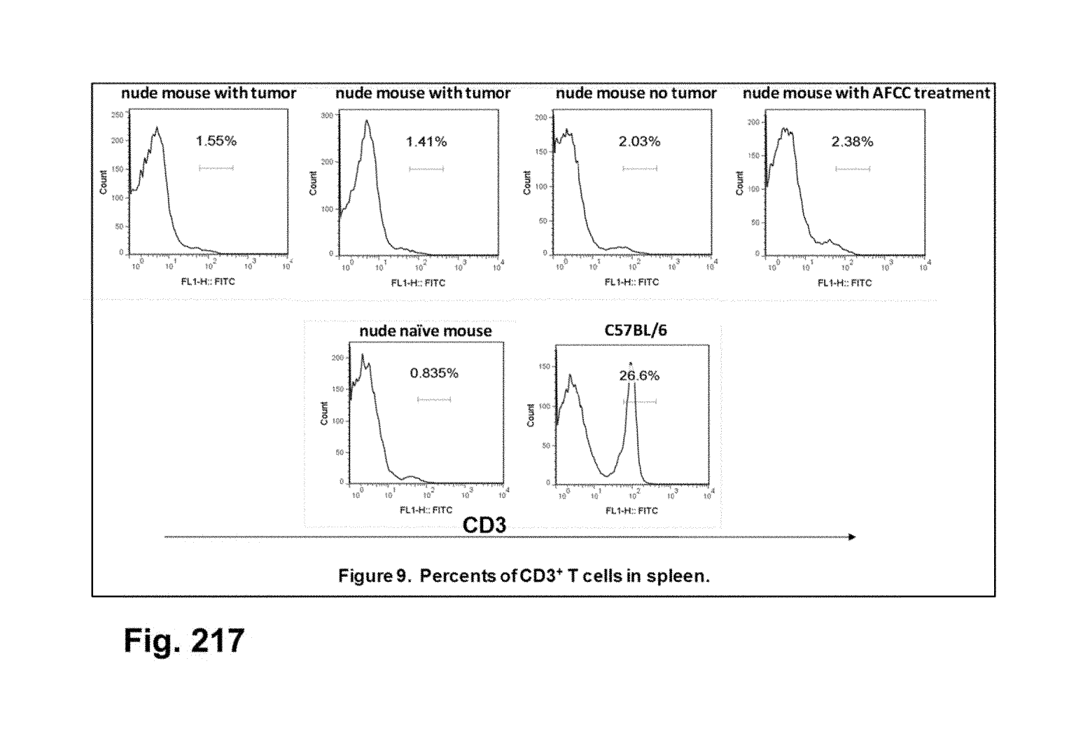

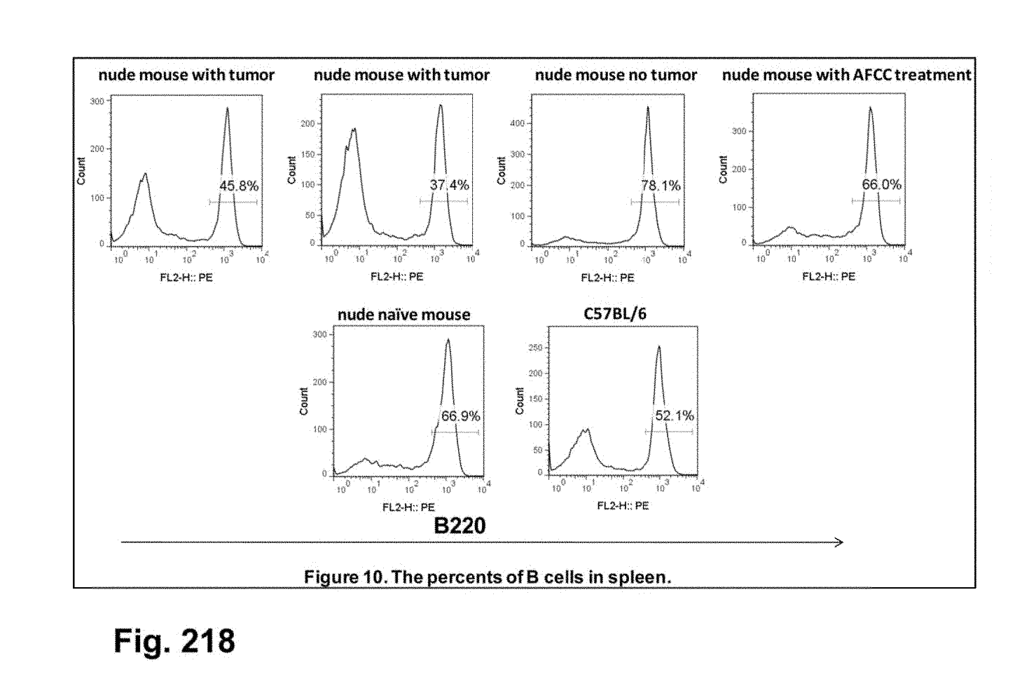

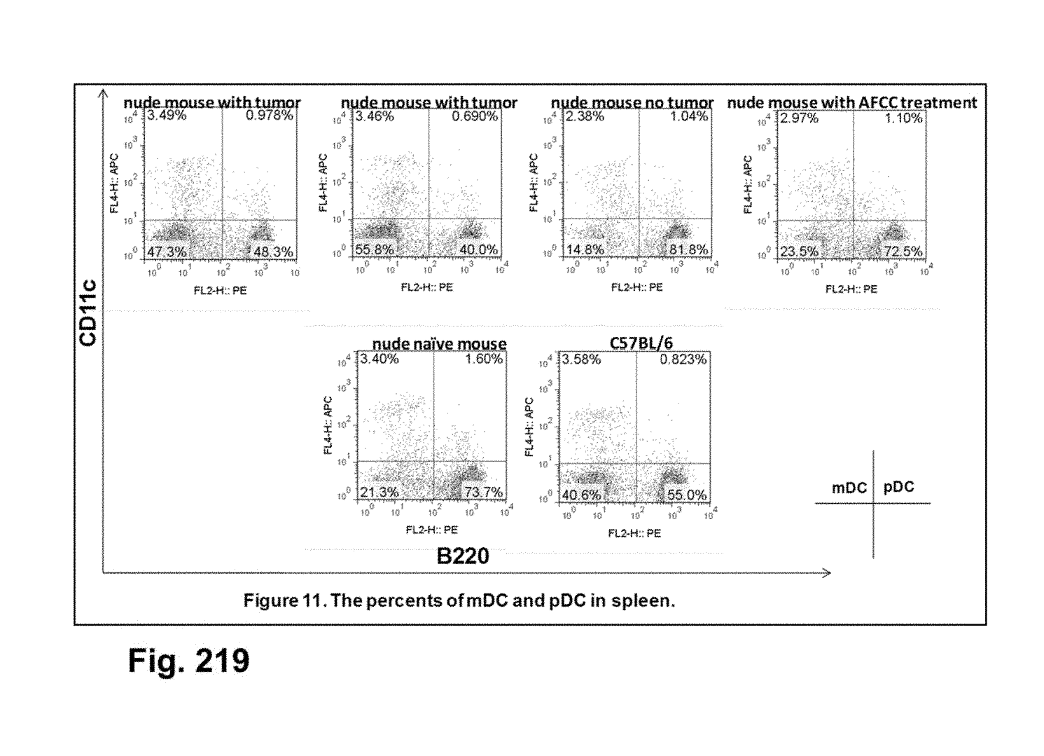

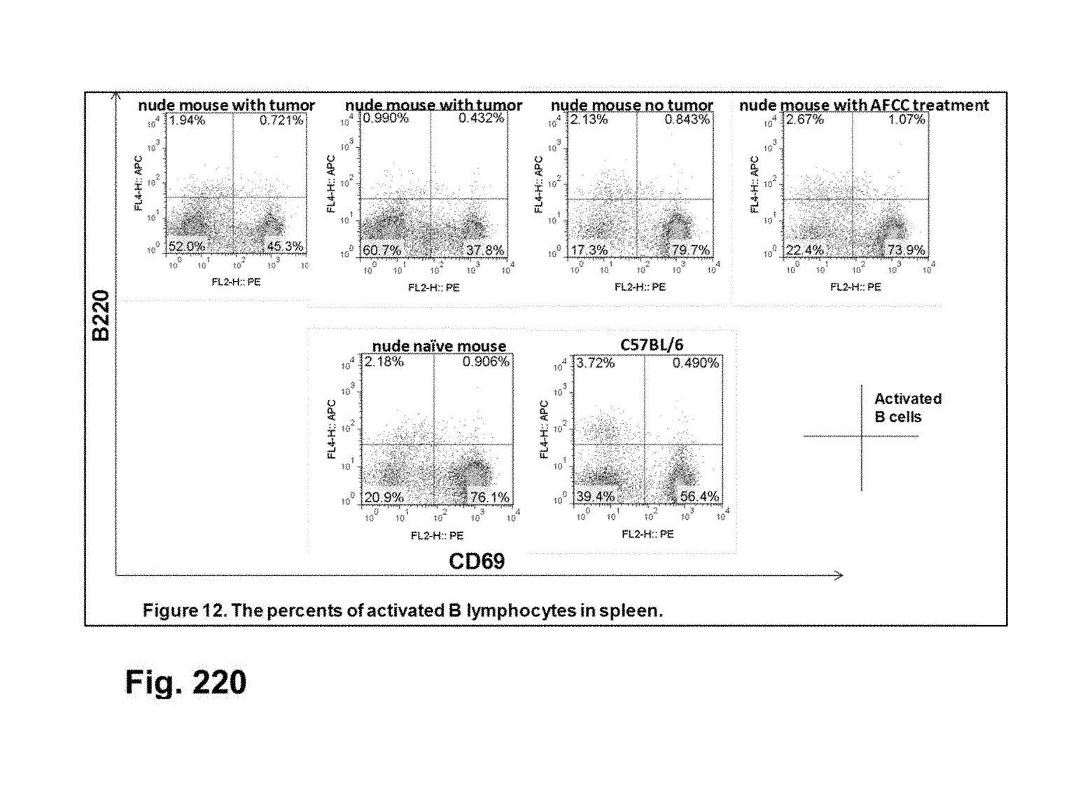

FIG. 212

Cell Population in Peripheral Blood

After whole blood withdrawal, distinct cell lineage was differentiated by cell surface marker proteins. T cells, B cells, activated B cells, mDC, pDC, granulocytes, and monocytes/macrophages were characterized (FIG. 3 to FIG. 8).

As shown by FIG. 3, AFCC treatment didn't affect CD3⁺T cell population compared with that in nude mouse with tumor and without tumor. After AFCC treatment, B cell population, on the other hand, increased to the similar percentage as seen in nude mouse no tumor and nude naïve mouse, suggesting the potential effect of AFCC on B cell lineage (FIG. 4). Activated B cells also increased with AFCC treatment, which was illustrated in FIG. 5. Macrophages and granulocytes decreased after AFCC treatment compared with those in nude mouse with tumor (FIG. 6 and FIG. 7). Nude mouse no tumor and nude mouse with AFCC treatment had similar mDC and pDC percentage shown in FIG. 8.

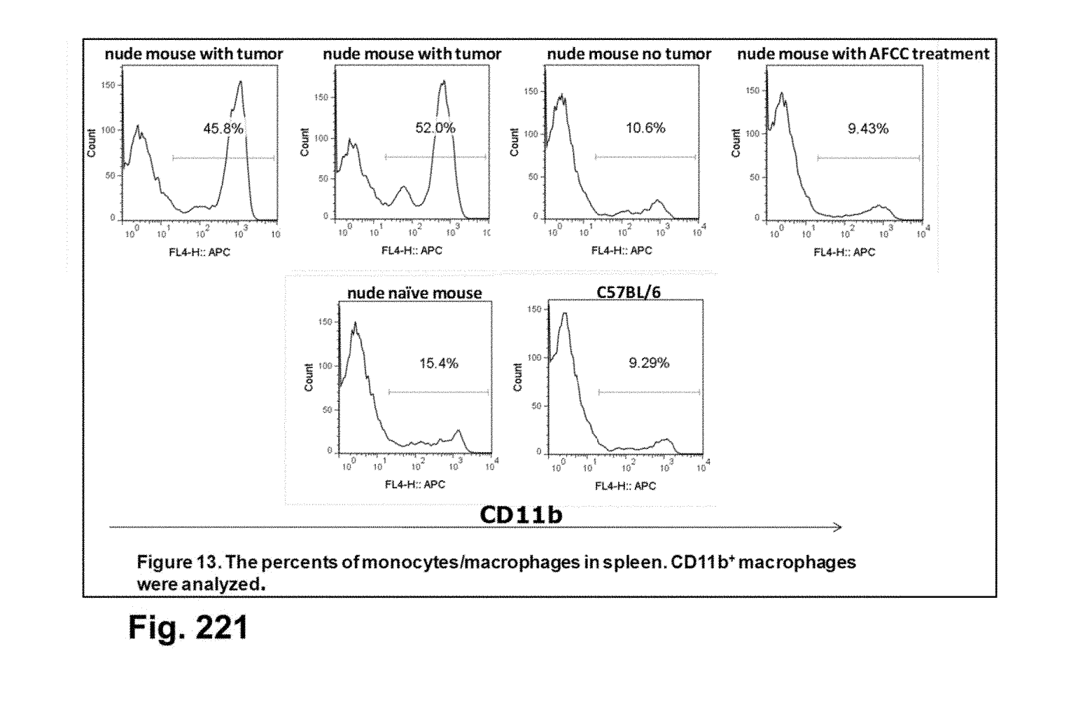

FIG. 213—The percents of B cells in peripheral blood.

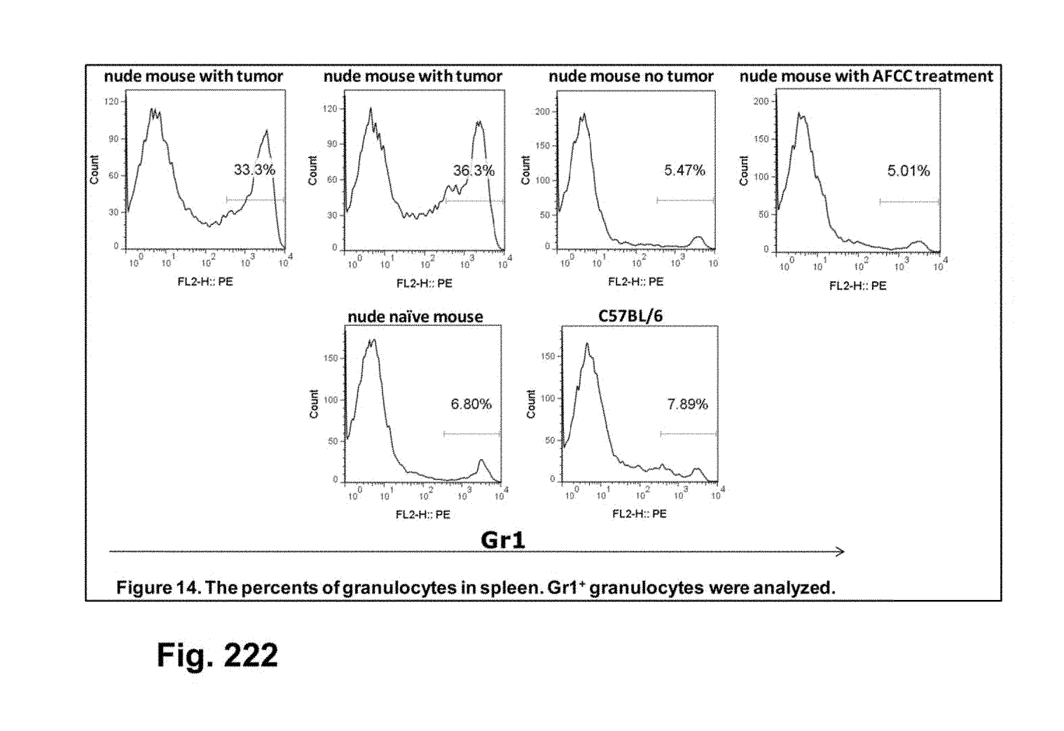

FIG. 214—The percents of activated B lymphocytes in peripheral blood.

FIG. 215—The percents of monocytes/macrophages in peripheral blood. CD11b macrophages were analyzed.

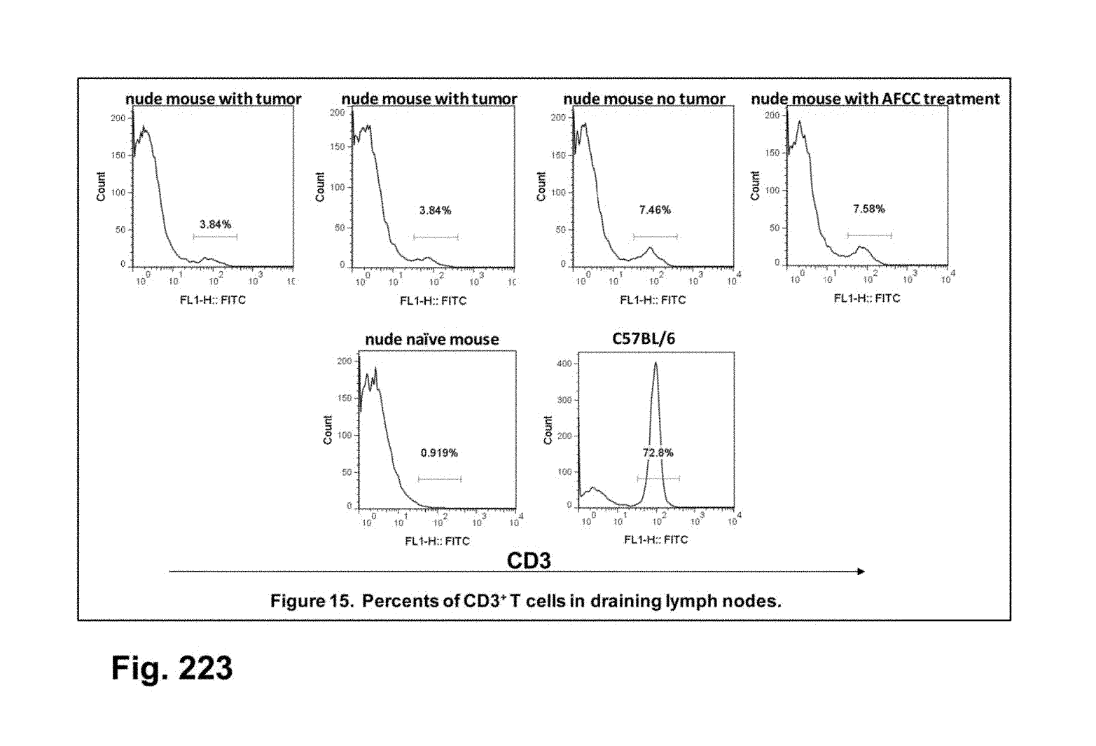

FIG. 216—The percents of mDC and pDC in peripheral blood.

Cell Population in Spleen

Distinct cell lineage in spleen cell suspension was further characterized by cell surface marker proteins. T cells, B cells, activated B cells, mDC, pDC, granulocytes, and monocytes/macrophages were included (FIG. 9 to FIG. 14).

As shown by FIG. 9, AFCC treatment slightly increased CD3⁺T cell population compared with that in nude mouse with tumor and nude mouse without tumor. After AFCC treatment, B cell population, on the other hand, increased to the similar percentage as seen in nude mouse no tumor, suggesting the potential effect of AFCC on B cell lineage (FIG. 10). Activated B cells also increased with AFCC treatment, which was illustrated in FIG. 12. Macrophages and granulocytes dramatically decreased after AFCC treatment compared with those in nude mouse with tumor (FIG. 13 and FIG. 14). Nude mouse no tumor and nude mouse with AFCC treatment had similar mDC and pDC percentage shown in FIG. 11.

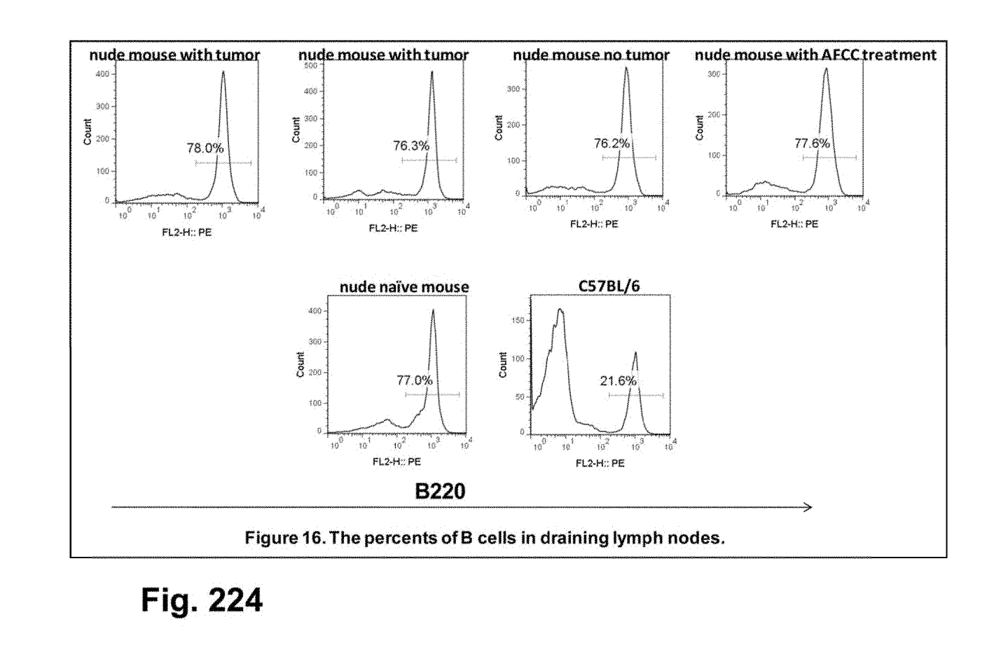

FIG. 217

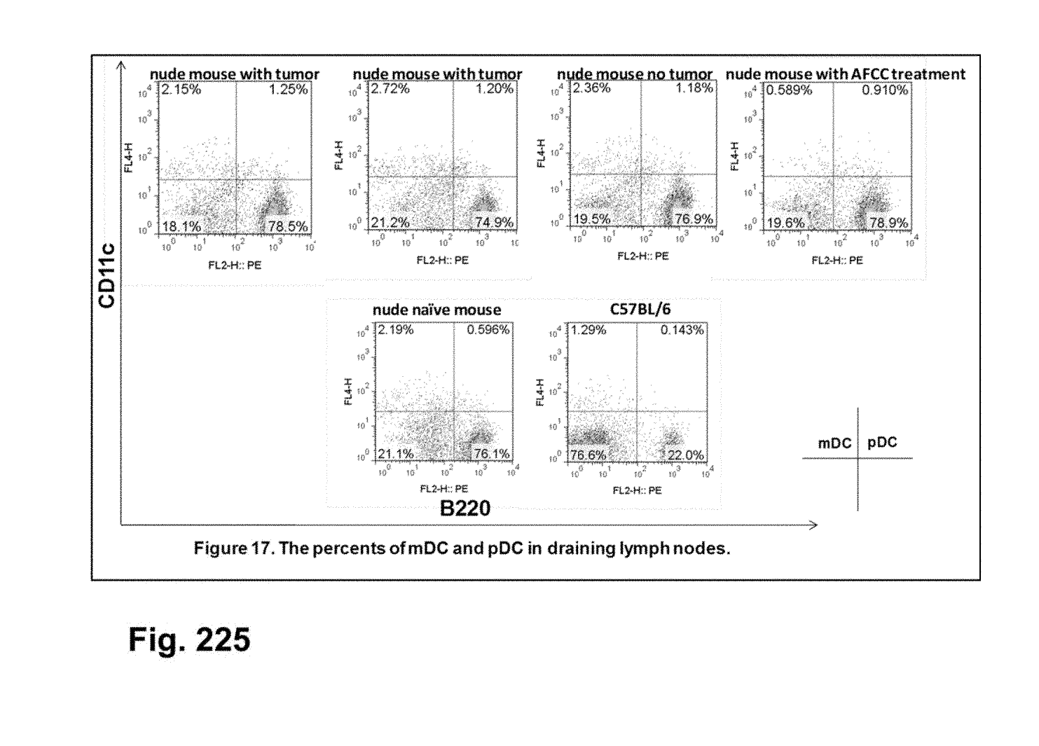

FIG. 218

FIG. 219

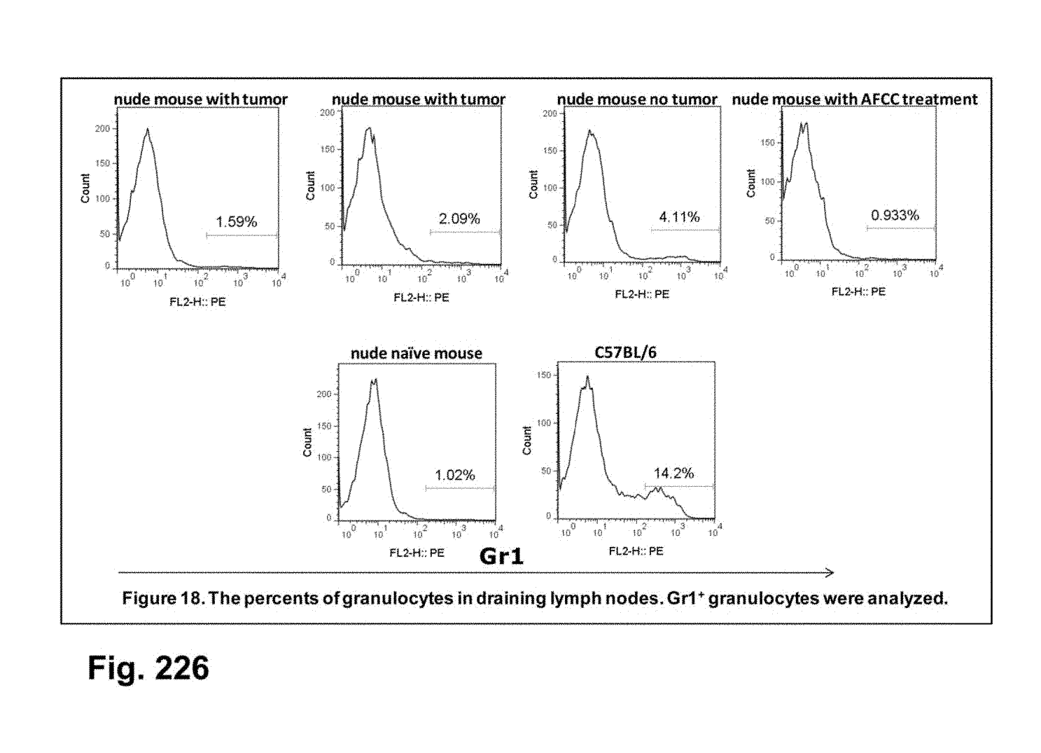

FIG. 220

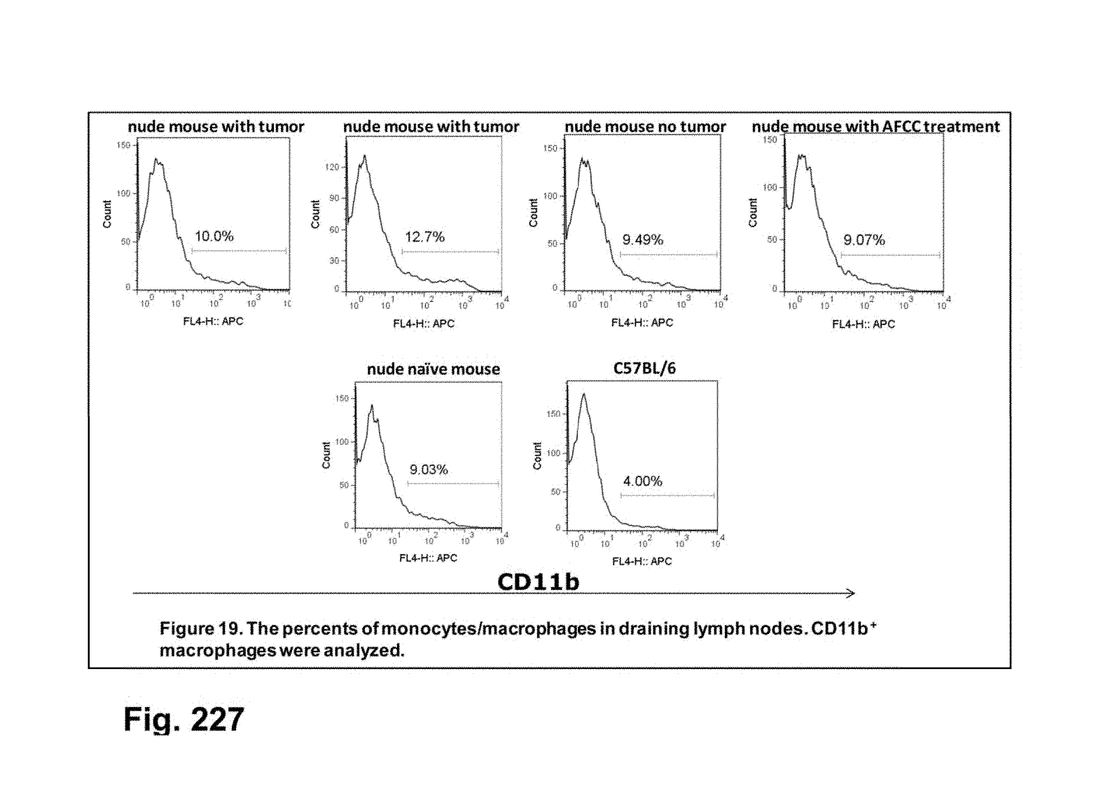

FIG. 221

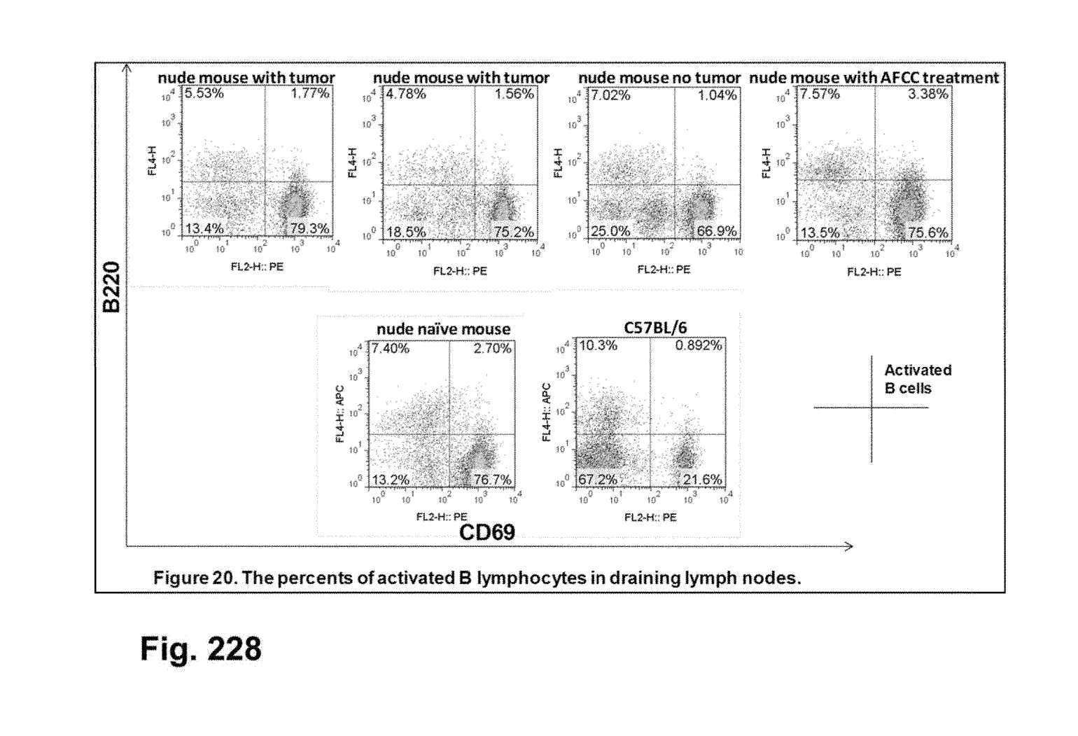

FIG. 222

Cell Population in Draining Lymph Nodes

Distinct cell lineage in draining lymph nodes suspension was further characterized by cell surface marker proteins. T cells, B cells, activated B cells, mDC, pDC, granulocytes, and monocytes/macrophages were included.

As shown by FIG. 15, AFCC treatment dramatically increased CD3⁺T cell population compared with that in nude mouse with tumor. T cells in nude mouse with AFCC treatment and mouse no tumor had the similar percentage (FIG. 15). After AFCC treatment, B cell population, on the other hand, increased to the similar percentage as seen in nude mouse no tumor, suggesting the potential effect of AFCC on B cell lineage (FIG. 16). Activated B cells also increased with AFCC treatment, which was illustrated in FIG. 20. Granulocytes dramatically decreased after AFCC treatment compared with those in nude mouse with tumor and naïve nude mouse (FIG. 18). mDC and pDC also decreased in the presence of AFCC compared to those in nude mouse with or without tumor (FIG. 17). Macrophages still maintained the similar percentage with and without AFCC treatment (FIG. 19).

FIG. 223

FIG. 224

FIG. 225

FIG. 226

FIG. 227

FIG. 228

Conclusions

The effect of AFCC on curing tumor through characterizing different cell lineage in lymphoid tissues and peripheral blood in nude mouse was investigated using staining with different marker proteins for distinct cell lineages followed by FACS. T cells, B cells, activated B cells, mDC, pDC, granulocytes, and monocytes/macrophages were characterized in 6 mice illustrated in FIG. 1 and FIG. 2.

FACS analysis showed that AFCC treatment had the effect on the population of major cell lineages in immune system. Increased CD3⁺T cell population was found in nude mouse treated with AFCC compared with that in nude mouse with tumor in spleen and lymph nodes (FIG. 9, 15). B cells including activated B cells also increased compared with that in nude mice with tumor in spleen, lymph nodes, and peripheral blood (FIG. 4, 10, 16, 5, 10, 20). Granulocytes and macrophages, however, were found to decrease after AFCC treatment in peripheral blood and spleen (FIGS. 7, 14, 18, 6, 13, and 19). The decrease as one of the lymphocytes, white blood cells, which are present in the peripheral blood of the nude mice with the breast cancer cell proves that the vehicle and positive control mice when the breast tumor grew the cancer cell have affected the peripheral blood. Even though the mice has not been metastasized. This make the inventor to believe that any cancer tumor grow the cancer cells are already in the peripheral blood.

Cells expressing KH proteins 1—Send signals to the cells contributing to disease, which triggers the synthesis of good proteins transforming these cells into healthy cells; 2—Send signals to the other currently undamaged cells to synthesize healthy proteins, which protect them from being damaged, infected and prone to DNA and other cellular alterations; 3—Send signals to the body to synthesize new healthy cells and inhibit them from being affected by intra- and extracellular damaging signals, thus treating and preventing disease, viral and bacterial infection, auto immune disease, neurological disorders, solid and blood cancers, and various other afflictions.

Macrophage populations have been found to decrease after AFCC treatment in peripheral blood and spleen; however it their incidence has not decreased in the vehicle and positive control mice.

Macrophages function in both non specific defense as well as help initiate specific defense mechanisms. Their role is to phagocytose, or engulf and then digest cellular debris and pathogens, either as stationary or as mobile cells. They also stimulate lymphocytes and other immune cells to respond to pathogens. They are present in all living tissues, and have a function in regeneration. The level of macrophages in the vehicle or positive control increases as the RNA of the damaged cells synthesize unhealthy proteins responsible in their contribution to cancer. Cells expressing healthy KH proteins help reduce the incidence and proliferation of breast cancer.

Taken together, this study suggests that AFCC plays a role in reducing tumors by changing the population of major cell lineages in the immune system, including the spleen, lymph nodes and peripheral blood.

Efficacy Study of AFOD RAAS 1 (APOA1) on Atherosclerosis:
1) APOE KO mice
2) LDLR KO mice
3) Rabbit

This study performed on APOE KO and LDLR KO mice and finally rabbits has shown, in APOE KO and LDLR KO mice, APOA1 is effective in the reduction and prevention of atherosclerosis.

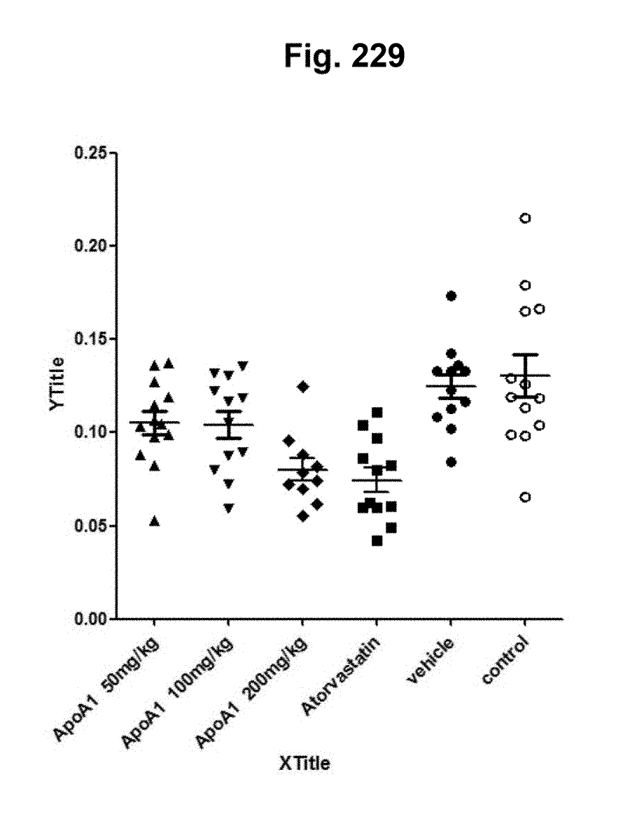

FIG. 229—APOE KO mice the area of atherosclerosis

The inhibition of inflammation factors RNA transcription.

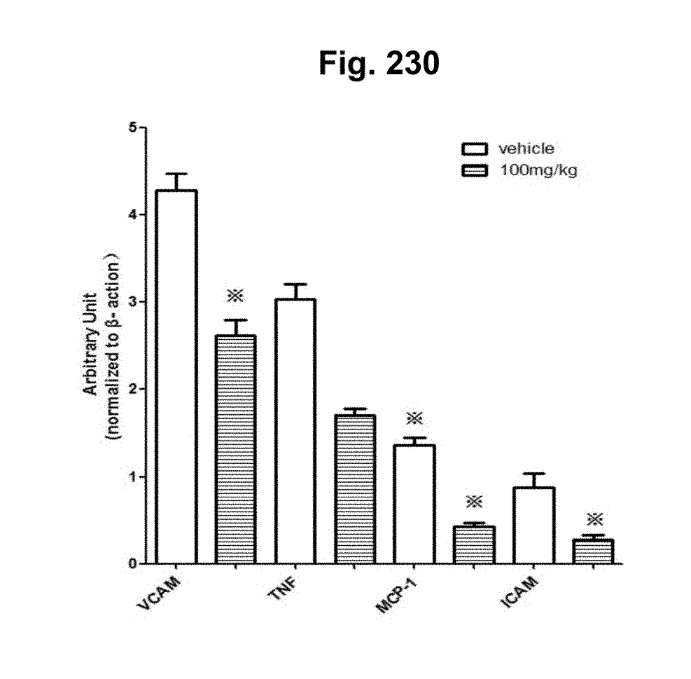

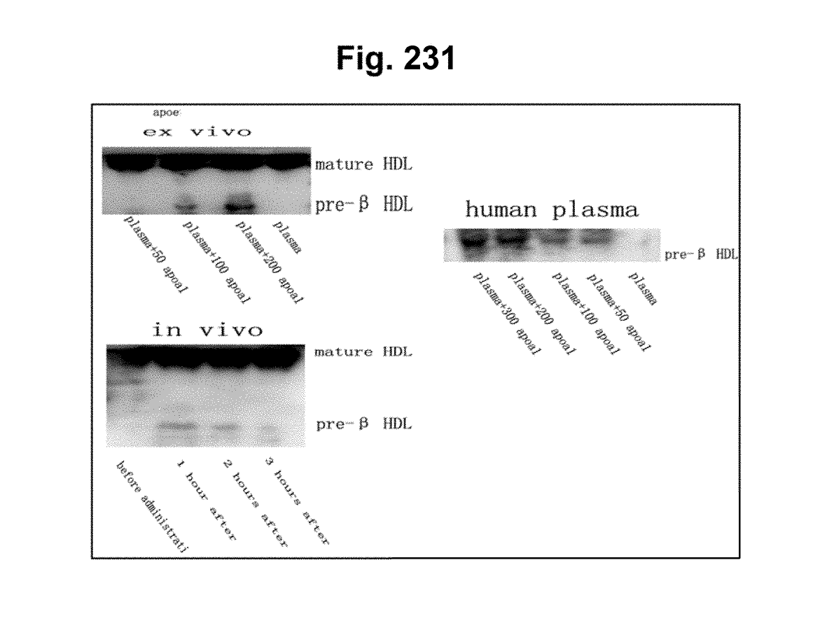

FIG. 230

| | | | Genebank |
|---|---|---|---|
| Gene | Forward | Reverse | ID |
| Lp- | GAGCGTCTTCGTGCGTTTG | GCGGGTATTTTTCTCCAGTC | NM_013737 |
| PLA2 | | | |
| MCP-1 | CCTGCTGTTCACAGTTGCC | TGTCTGGACCCATTCCTTCT | NM_011333 |
| ICAM- | GCTGTATGGTCCTCGGCTG | GCCCACAATGACCAGCAGTA | NM_010494 |
| 1 | | | |
| VCAM- | TGAACCCAAACAGAGGCAGA | CGGAATCGTCCCTTTTTGTAG | NM_011693 |
| 1 | | | |
| MMP-2 | CAAAGAAAGGTGCTGACTGT | GAAGGAAACGAGCGAAGG | NM_008610 |
| MMP-9 | CAGCCAACTATGACCAGGAT | TGCCGTCTATGTCGTCTTTA | NM_013599 |
| TNF-α | GGGCAGGTCTACTTTGGAG | AGCCCATTTGAGTCCTTGAT | NM_13693 |
| β-Actin | GGGAAATCGTGCGTGACA | CAAGAAGGAAGGCTGGAAAA | NM_007393 |

FIG. 231—The increase of pre-B HDL after injection of APOA1 Protein—Western blot of pre-b HDL

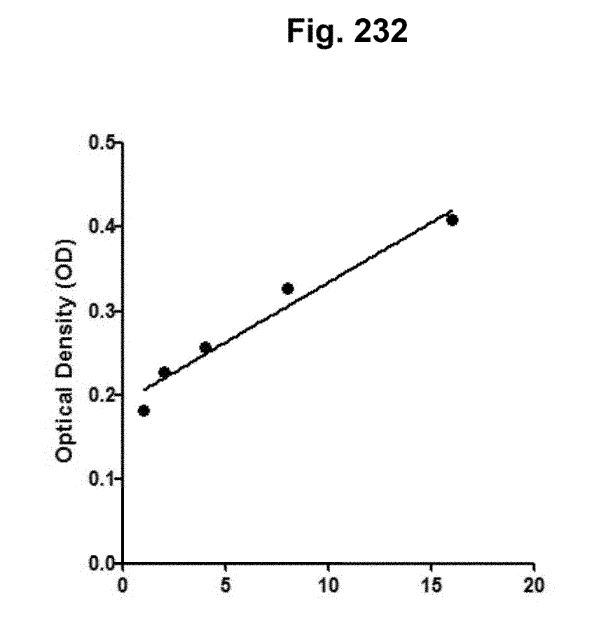

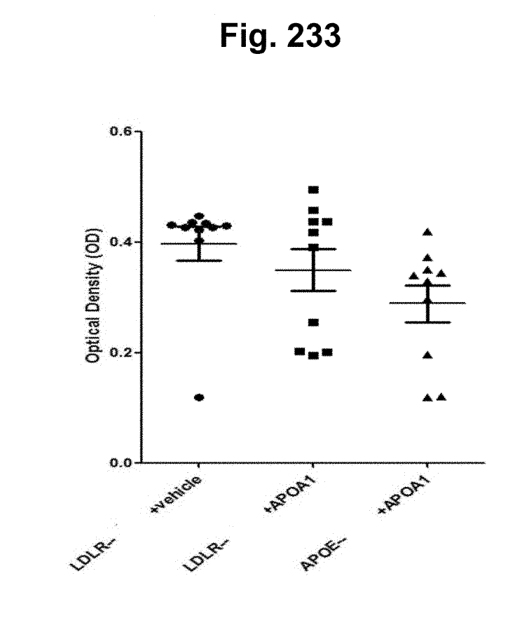

FIG. 232, 233—There is no antibody of APOA1 raised in mice

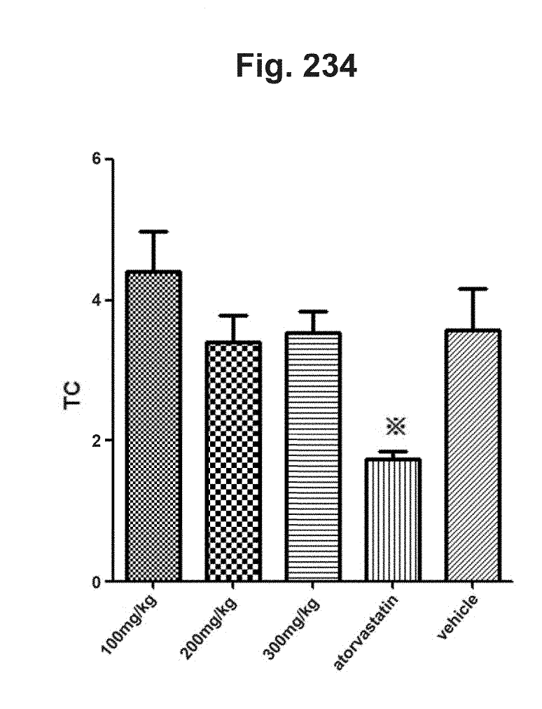

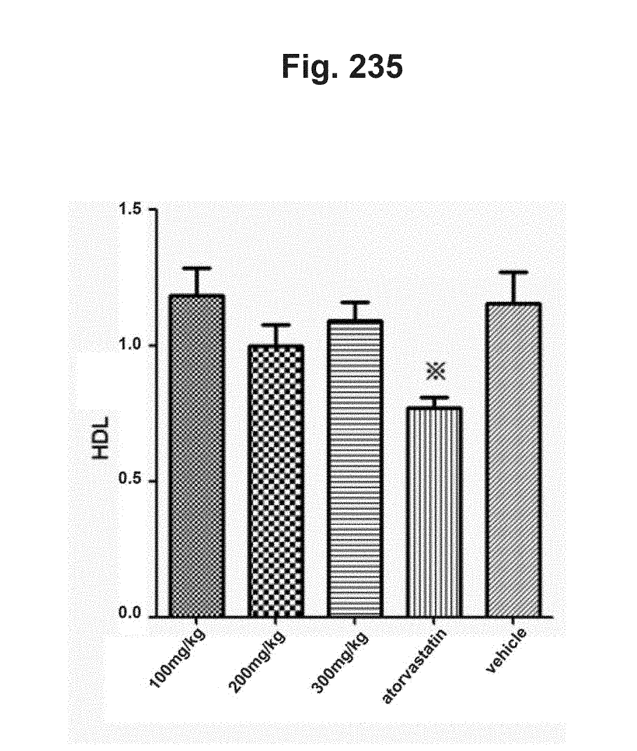

FIG. 234, 235—Lipid change in LDLR knock out mice

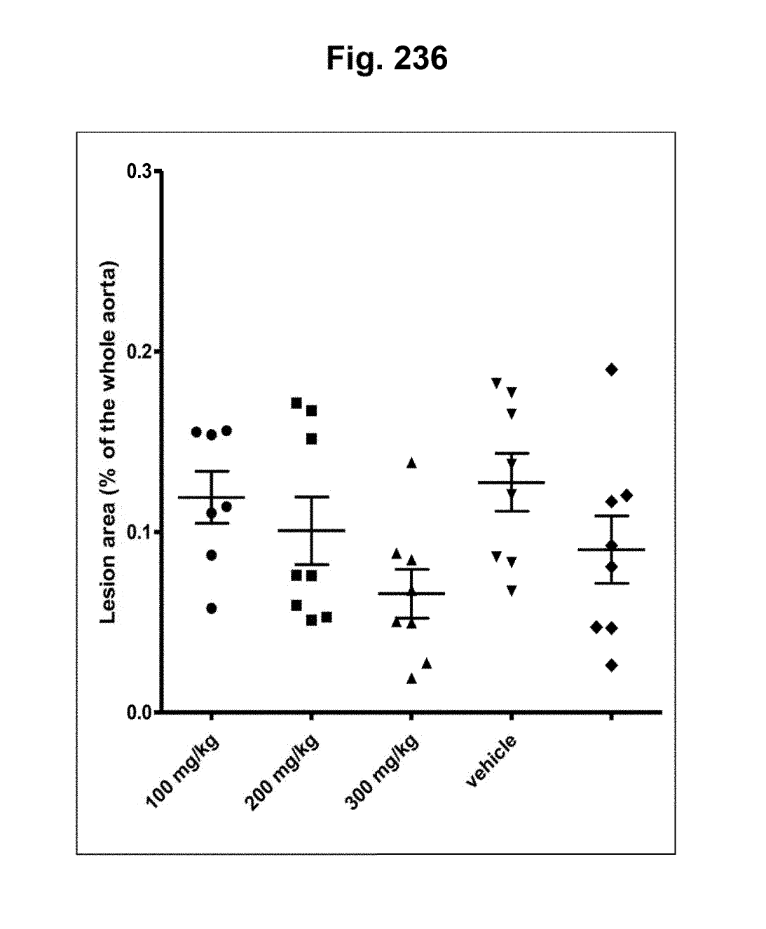

FIG. 236—LDLR KO mice—the area of atherosclerosis

AFOD RAAS 1 (APOAI) Efficacy Pilot Study in 6-OHDA Rat PD Model

Animal Model

I. Animal Description
Species: Rat
Strain: SD rat, SPF grade
Age or weight: 6 to 8 weeks
Sex: Male

II. Experiment Outline
Objectives: To test the effects of AFOD RAAS 1 (APOA1) product on 6-OHDA rat model of Parkinson's disease (PD).
Experiment design:
6-OHDA Lesion:
Each rat will receive an injection of 50 μg 6-OHDA into the both sides of medial forebrain bundle to induce severe lesion n of the dopamine system. This high dose will cause the death of the rats at around 15 days after lesion. RAAS product will be injected daily right after lesion.
Group: vehicle, n=6; AFOD, n=6

III. Preliminary Result
One rat survived in AFOD group and all vehicle rats died. The second batch of animal experienced the same mortality. The body weight of AFOD group rat increased significantly.
Then rats were sent to Ruijin Hospital for PET/CT scanning to determine the function recovery of damaged brain.
Equipment: Siemens Inveon
PET/CT scanning results: There has been signal improvement in the brain of the two rats left, in the first batch and in the second batch to compare with the two normal control rats.

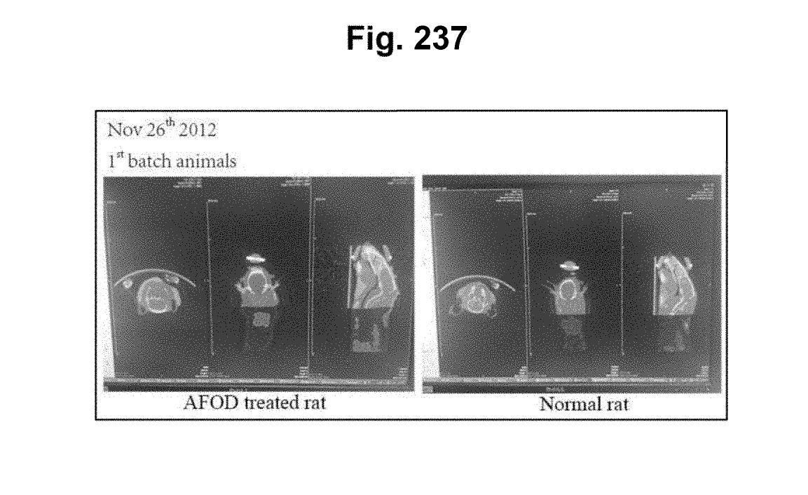

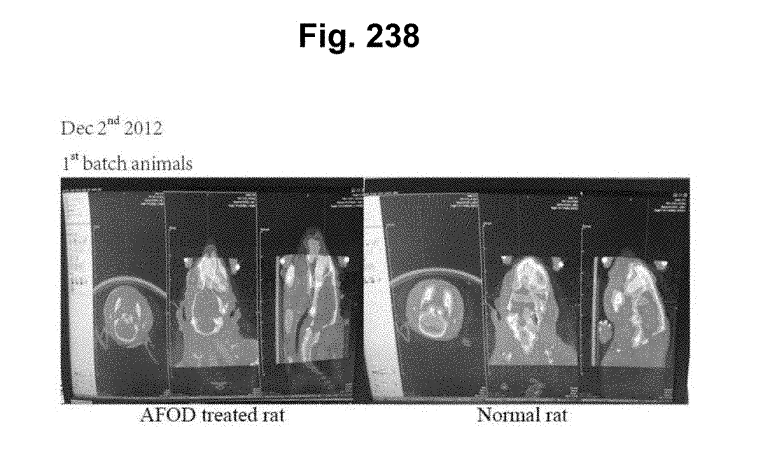

FIG. 237—PET/CT scans

FIG. 238—PET/CT scans

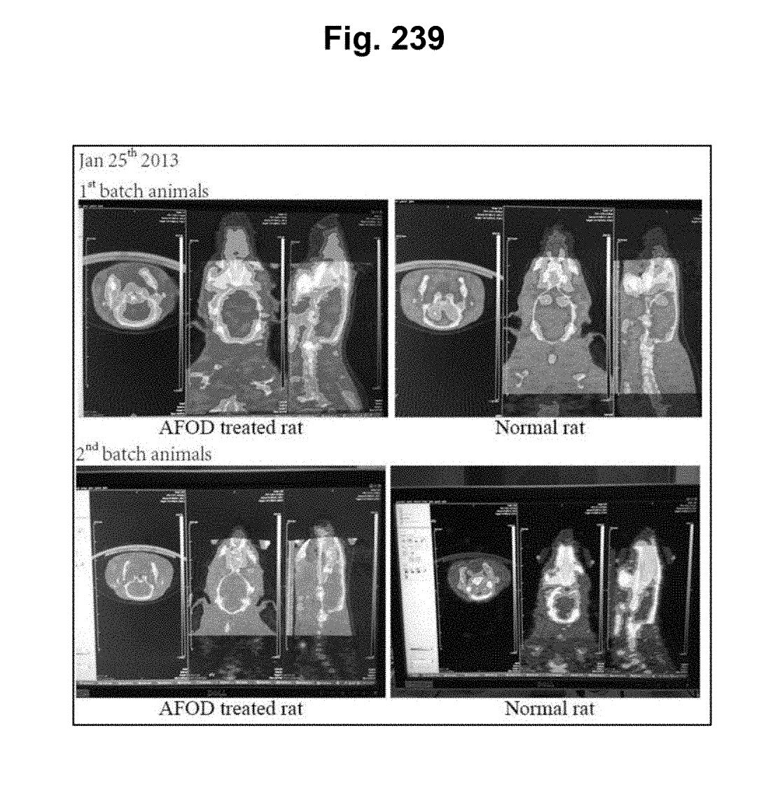

FIG. 239—PET/CT scans

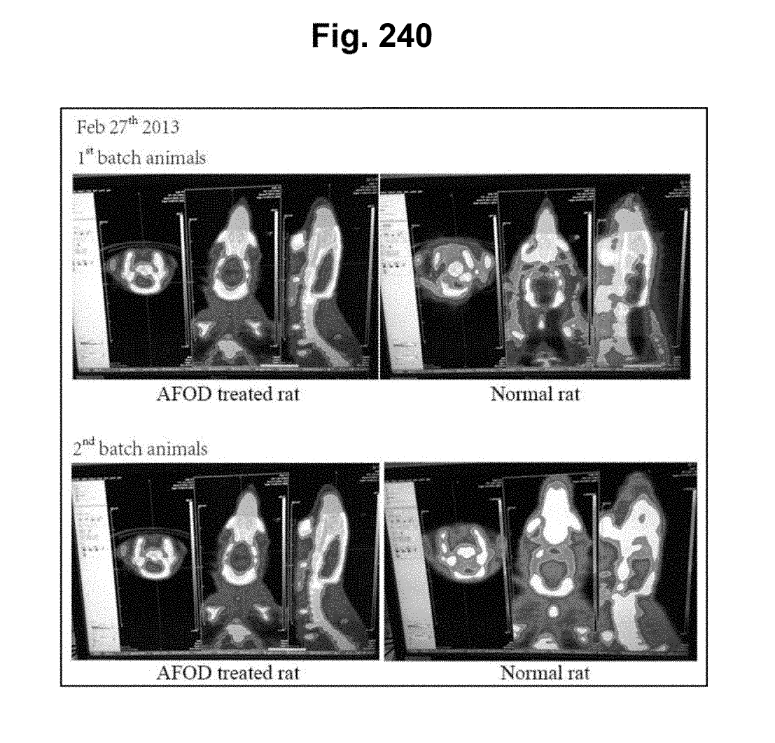

FIG. 240—PET/CT scans

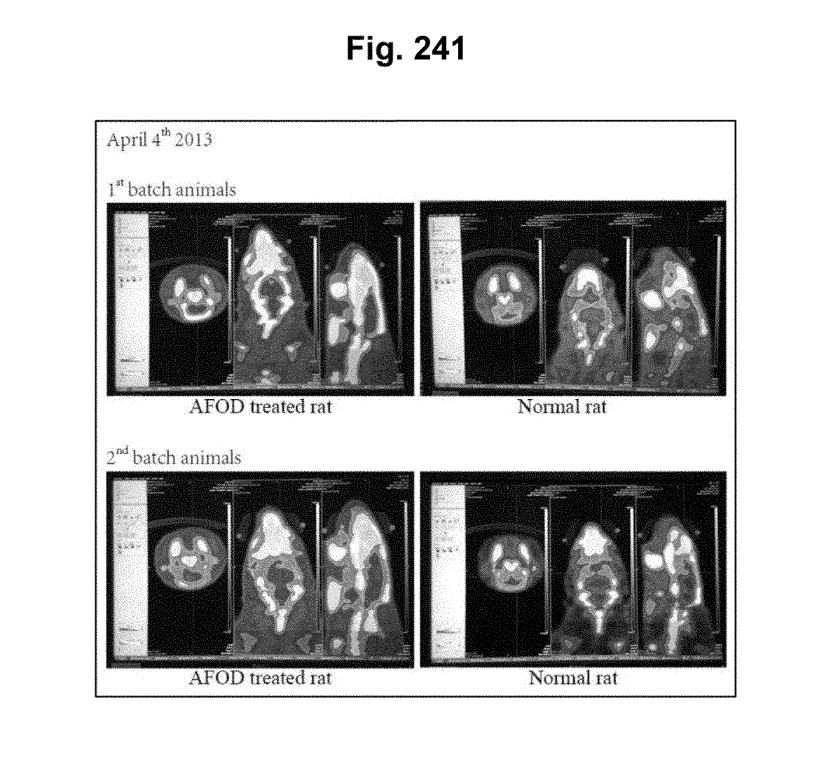

FIG. 241—PET/CT scans

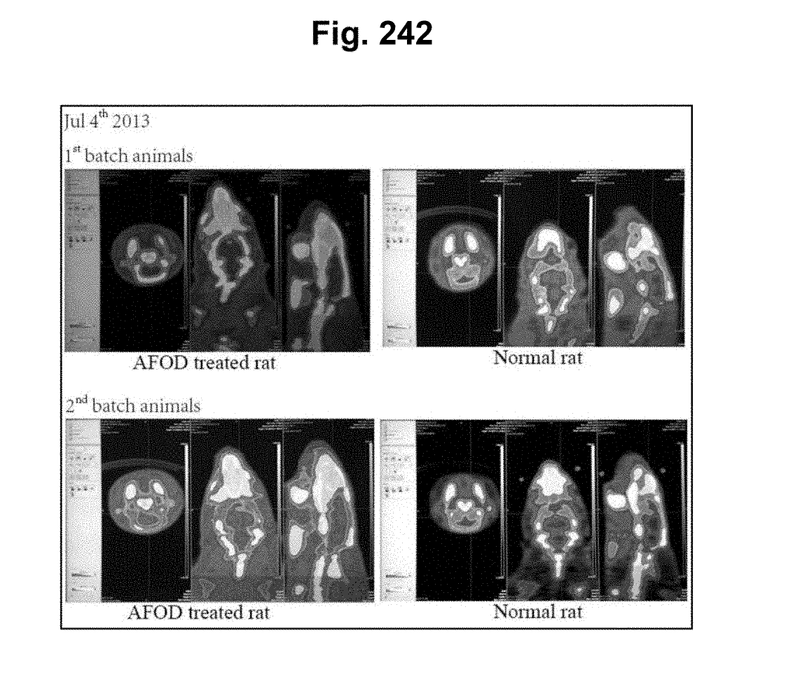

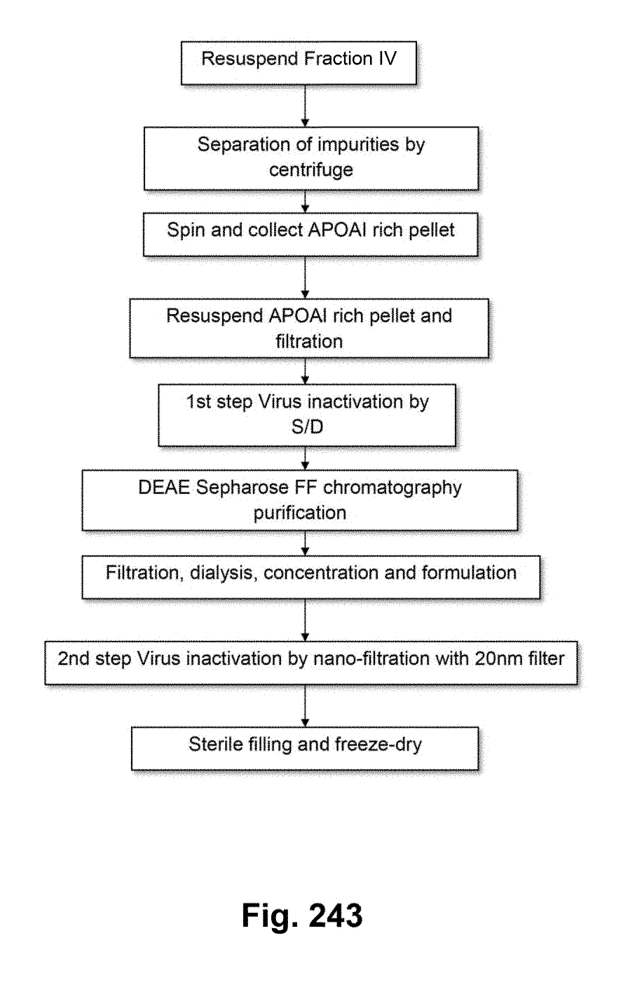

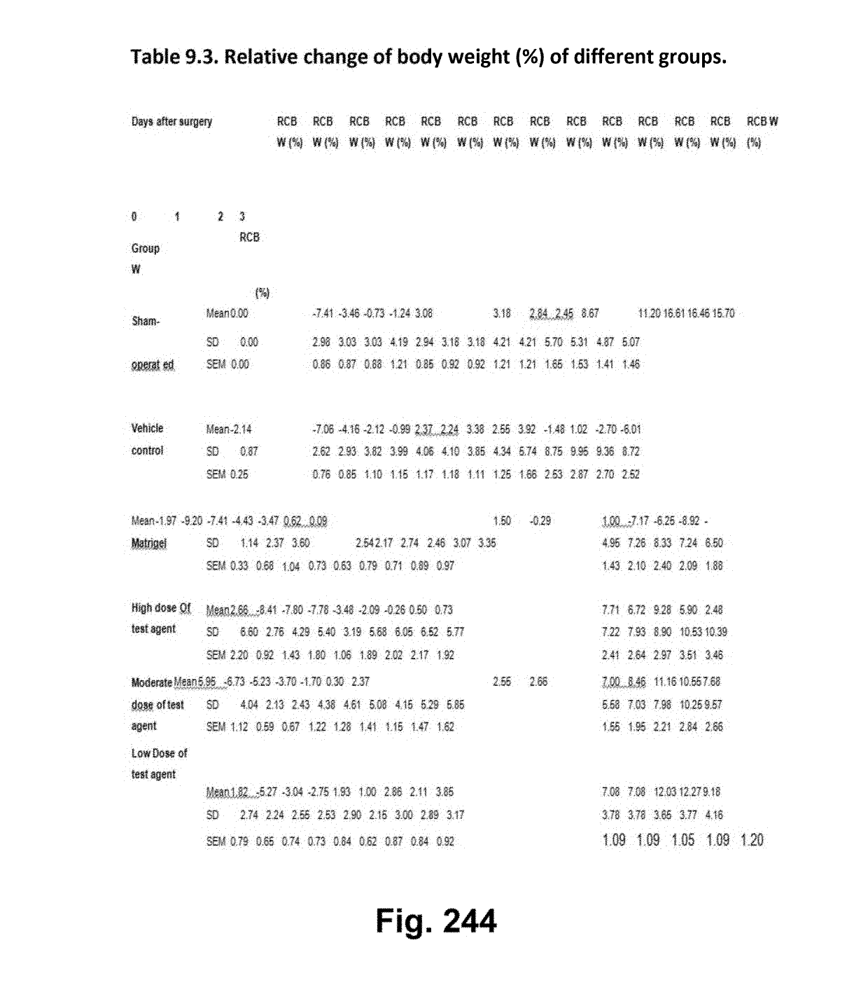

FIG. 242—PET/CT scans

1. A method of treating or preventing disease and infection in a mammal comprising, administering to the mammal a composition, compound, or solution containing an effective amount of at least one isolated purified plasma product selected from the group consisting of:
cryoprecipitate;
fraction III;
fraction III-II;
fraction IV;
prothrombin;
human factor VIII;
human fibrinogen;
human immunoglobulin;
human thrombin;
human albumin; and
transferrin.

2. The method according to claim 1 wherein the at least one isolated purified plasma product further comprises at least one protein defined by an amino acid sequence selected from the group consisting of: SEQ ID NOs 1-55.

3. A method of treating human immunodeficiency virus comprising, administering to an individual infected with HIV a composition, compound, or solution containing an effective amount of a blood plasma product comprising purified factor II, purified factor VII, and purified factor X.

4. The method according to claim 3, wherein the composition, compound, or solution has a concentration of at least 3.5% of at least one protein defined by an amino acid sequence selected from the group consisting of SEQ ID NOs 1-55.

5. The method according to claim 3, wherein composition, compound, or solution has a concentration of at least 400 ug/ml.

6. A method of treating human immunodeficiency virus comprising, administering to an individual infected with HIV a composition, compound, or solution containing an effective amount of a blood plasma product comprising purified antithrombin III and at least one protein defined by an amino acid sequence selected from the group consisting of:
SEQ ID NO: 21;
SEQ ID NO: 22;
SEQ ID NO: 23;
SEQ ID NO: 24;
SEQ ID NO: 25;
SEQ ID NO: 26;
SEQ ID NO: 27;
SEQ ID NO: 48;
SEQ ID NO: 49; and
SEQ ID NO: 50.

7. The method of claim 6, wherein the composition, compound, or solution has a concentration of at least 15% of at least one protein selected from the group consisting of:
CP 98 kDa;
CP Ceruloplasmin;
KRT2 Keratin, type II cytoskeletal 2 epidermal;
APOA1;
human albumin;
transferrin;
vimentin; and
Haptoglobin.

8. A method of treating hepatitis C virus in a mammal comprising, administering to said mammal a composition, compound, or solution containing an effective amount of a blood plasma concentrate containing:
CP 98 kDa;
CP Ceruloplasmin;
KRT2 Keratin, type II cytoskeletal 2 epidermal;
APOA1;
human albumin;
transferrin; and
haptoglobin.

9. The method of claim 8, wherein the blood plasma concentrate comprises at least 10% of the composition, compound, or solution.

10. The method of claim 8, wherein the blood plasma concentrate has a concentration of at least 400 ug/ml.

11. A method of treating hepatitis C virus in a mammal comprising, administering to said mammal a composition, compound, or solution containing an effective amount of a blood plasma concentrate comprising factor II, factor VII, factor IX, and factor X.

12. The method of claim 11, wherein the blood plasma concentrate comprises at least 4% of the composition, compound, or solution.

13. The method of claim 11, wherein the blood plasma concentrate has a concentration of at least 400 ug/ml.

14. A method of treating hepatitis B virus in a mammal comprising, administering to said mammal a composition, compound, or solution containing an effective amount of a blood plasma concentrate comprising:
CP 98 kDa;
CP Reuloplasmin;
KRT2 Keratin, type II cytoskeletal epidermal;
a protein defined by amino acid sequence SEQ ID NO: 22;
a protein defined by amino acid sequence SEQ ID NO: 23;
a protein defined by amino acid sequence SEQ ID NO: 24;
a protein defined by amino acid sequence SEQ ID NO: 25;
APOA1;
human albumin;
transferrin;
vimentin; and
haptoglobin.

15. The method of claim 14, wherein the blood plasma concentrate has a concentration of at least 1.25 ug/ml.

16. The method of claim 14, wherein the blood plasma concentrate has a concentration of at least 10 ug/ml.

17. A method of treating hepatitis B virus in a mammal comprising, administering to said mammal a composition, compound, or solution containing an effective amount of a blood plasma concentrate derived from fraction III IVIG, the blood plasma concentrate derived from fraction III IVIG comprising TF serotransferrin.

18. The method of claim 17, wherein the blood plasma concentrate comprises at least 25% of the composition, compound, or solution.

19. A method of treating influenza in a mammal comprising, administering to said mammal a composition, compound, or solution containing an effective amount of a purified plasma product, wherein said purified plasma product is selected from the group consisting of:
a first protein concentrate comprising proteins CP 98 kDa, CP Ceruloplasmin, KRT2 Keratin-type II cytoskeletal 2 epidermal, APOA1, human albumin, transferrin, vimentin, and haptoglobin; and
a prothrombin complex protein concentrate comprising proteins factor II, factor VII, factor IX, and factor X.

20. The method of claim 19, wherein an effective dose of the purified plasma product is administered to the mammal for at least two weeks.

21. The method of claim 19, wherein the first protein concentrate comprises at least 10% of the composition, compound, or solution.

22. The method of claim 19, wherein the prothrombin complex protein concentrate comprises at least 0.0020% of the composition, compound, or solution.

23. The method of claim 19, wherein the first protein concentrate has a concentration of at least 69.06 ug/ml.

24. A method of treating diabetes mellitus in a mammal comprising, administering to said mammal a composition, compound, or solution containing an effective amount of a purified plasma product, wherein the purified plasma product is selected from the group consisting of:
a first protein concentrate comprising protein 1CP 98 kDa, wherein protein 1CP98 kDa containing Nup98 and Nup96;
a second protein concentrate comprising transferrin; and
a third protein concentrate comprising CP 98 kDa, CP Ceruloplasmin, KRT2 Keratin type II cytoskeletal 2 epidermal, APOA1, human albumin, transferrin, vimentin, and haptoglobin.

25. The method according to claim 24, wherein the second protein concentrate further comprises at least one protein having an amino acid sequence selected from the group consisting of SEQ ID NOs 21-27 and 48-50.

26. The method of claim 24, wherein the first protein concentrate comprises at least 0.05% of the composition, compound, or solution.

27. The method of claim 24, wherein the second protein concentrate comprises at least 0.1% of the composition, compound, or solution.

28. The method of claim 24, wherein third protein concentrate comprises at least 0.1% of the composition, compound, or solution.

29. A method of treating and preventing atherosclerosis and related cardiovascular diseases comprising, administering to an individual a daily dose of a composition, compound, or solution containing an effective amount of purified Apolipoprotein A-1 for at least 16 weeks.

30. The method of claim 29, wherein Apolipoprotein A-1 comprises at least 5% protein CPD by concentration.

31. A method of treating cancer in a mammal comprising, administering to said mammal a composition, compound, or solution containing an effective amount of at least one plasma product selected from the group consisting of: high concentrated fibrinogen, enriched a1at, thrombin, and AFOD.

32. The method according to claim 31 further comprising:
a) surgically exposing a tumor; and
b) coating the tumor and a peritoneal surface surrounding the tumor with the composition, compound, or solution containing the at least one plasma product.

33. A method of treating cancer in a mammal comprising, administering to a mammal a composition, compound, or solution containing an effective amount of high concentrated fibrinogen enriched a1at thrombin and AFOD.

34. The method according to claim 33 further comprising:
a) surgically exposing the tumor; and
b) coating the tumor and a peritoneal surface surrounding the tumor with the composition, compound, or solution containing the at least one plasma product.

35. The method according to claim 33, wherein the cancer being treated is diagnostically associated as colorectal, lung, hepatic, ovarian, or breast in origin.

36. The method according to claim 35, wherein the cancer being treated is diagnostically associated as colorectal, lung, hepatic, ovarian, or breast in origin.

37. A method of treating Parkinson's disease in a mammal comprising:
a) performing a PET/CT scan on said mammal to determine baseline brain function;
b) intravenously administering an effective daily dose of a ApoAI to said mammal;
c) performing at least one additional PET/CT scan;
d) determining whether brain signal function has improved in a time period between performing the at least one additional PET/CT scan and the previous PET/CT scan; and
e) discontinuing intravenous administration of ApoAI to said mammal once it is determined that brain signal function has not improved in the time period between performing the at least one additional PET/CT scan and the previous PET/CT scan.


SEQUENCE LISTING (54 sequences)

SEQ ID NO: 1 - protein, 775 aa, Homo sapiens
MDTYIESHFAGALAYRDLIKVLKWYVDRITEAERQEHIQEVLKAQEYIFKYIVQSRRLFSLATGGQNEEEFRCCIQELLMSVRFFLSQESKGSGALSQSQAVFLSSFPAVYSELLKLFDVREVANLVQDTLGSLPTILHVDDSLQAIKLQCIGKTVESQLYTNPDSRYILLPVVLHHLHIHLQEQKDLIMCARILSNVFCLIKKNSSEKSVLEEIDVIVASLLDILLRTILEITSRPQPSSSAMRFQFQDVTGEFVACLLSLLRQMTDRHYQQLLDSFNTKEELRDFLLQIFTVFRILIRPEMFPKDWTVMRLVANNVIITTVLYLSDALRKNFLNENFDYKIWDSYFYLAVIFINQLCLQLEMFTPSKKKKVLEKYGDMRVTMGCEIFSMWQNLGEHKLHFIPALIGPFLEVTLIPQPDLRNVMIPIFHDMMDWEQRRSGNFKQVEAKLIDKLDSLMSEGEGDETYRELFNSIIPLFGPYPSLLKKIERETWRESGVSLIATVTRLMERLLDYRDCMKMGEVDGKKIGCTVSLLNFYKTELNKEEMYIRYIHKLYDLHLKAQNFTEAAYTLLLYDELLEWSDRPLREFLTYPMQTEWQRKEHLHLTIIQNFDRGKCWENGIILCRKIAEQYESYYDYRNLSKMRMMEASLYDKIMDQQRLEPEFFRVGFYGKKFPFFLRNKEFVCRGHDYERLEAFQQRMLNEFPHAIAMQHANQPDETIFQAEAQYLQIYAVTPIPESQEVLQREGVPDNIKSFYKVNHIWKFRYDRPFHKGT

SEQ ID NO: 2 - protein, 241 aa, Homo Sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLN

SEQ ID NO: 3 - protein, 243 aa, Homo Sapiens
DEPPQSPWDRVKDLATVYVDVLKDSGRDYVSQFEGSALGKQLNLKLLDNWDSVTSTFSKLREQLGPVTQEFWDNLEKETEGLCQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 4 - protein, 72 aa, Homo sapiens
  misc_feature (12)..(12): Xaa can be any naturally occurring amino acid
MGCKRASEVCGXAVEGLRDPLKPSEPSQGAAGKRKGTEYLMKQKLEFGGRGEELLLGVHLRGAQKTGGGWRR

SEQ ID NO: 5 - protein, 605 aa, Homo sapiens
MAHVRGLQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 6 - protein, 375 aa, Homo sapiens [sequence omitted]

SEQ ID NO: 7 - protein, 595 aa, Homo sapiens [sequence omitted]

SEQ ID NO: 8 - protein, 439 aa, Homo sapiens
TPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 9 - protein, 371 aa, Homo sapiens
MEPRAVGVSKQDIREQIWGYMESQNLADFPRPVHHRIPNFKGSYLACQNIKDLDVFARAQEVKVDPDKPLEGVRLLVLQSKKTLLVPTPRLRTGLFNKITPPPGATKDILRKCATSQGVRNYSVPIGLDSRVLVDLVVVGSVAASEKGWRIGKGEGYADLEYAMMVSMGAVSKETPVVTIVHDCQVVDIPEELVEEHDITVDYILTPTRVIATGCKRPKPMGITWFKISLEMMEKIPILRSLRAREQQAGKDVTLQGEHQHLPEPGCQQTVPLSVGRRPPDTPGPETNSMEAAPGSPPGEGAPLAADVYVGNLPRDARVSDLKRALRELGSVPLRLTWQGPRRRAFLHYPDSAAASRPSPACRACAWAPTP

SEQ ID NO: 10 - protein, 142 aa, Homo sapiens [sequence omitted]

SEQ ID NO: 11 - protein, 243 aa, Homo sapiens; identical to SEQ ID NO: 3

SEQ ID NO: 12 - protein, 622 aa, Homo sapiens [sequence omitted]

SEQ ID NO: 13 - protein, 415 aa, Homo sapiens [sequence omitted]

SEQ ID NO: 14 - protein, 427 aa, Homo sapiens [sequence omitted]

SEQ ID NO: 15 - protein, 427 aa, Homo sapiens [sequence omitted]

SEQ ID NO: 16 - protein, 427 aa, Homo sapiens [sequence omitted]

SEQ ID NO: 17 - protein, 347 aa, Homo sapiens
MDDDIAALVVDNGSGMCKAGFAGDDAPRAVFPSIVGRPRHQGVMVGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 18 - protein, 356 aa, Homo sapiens
MEEEIAALVIDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 19 - protein, 248 aa, Homo sapiens
MQKSEGSGGTQLKNRATGNYDQRTSSSTQLKHRNAVQGSKSSLSTSSPESARKLHPRPSDKLNPKTINPFGEQSRVPSAFAAIYSKGGIPCRLVHGSVKHRLQWECPPESLSFDPLLITLAEGLRETKHPYTFVSKEGFRELLLVKGAPEKAIPLLPRLIPVLKAALVHSDDEVFERGLNALVQLSVVVGPSLNDHLKHLLTSLSKRLMDKKFKEPITSALQKLEQHGGSGSLSIIKSKIPTYCSICC

SEQ ID NO: 20 - protein, 215 aa, Homo sapiens
MASETEKTHALLQTCSTESLISSLGLGAFCLVADRLLQFSTIQQNDWLRALSDNAVHCVIGMWSWAVVTGIKKKTDFGEIILAGFLASVIDVDHFFLAGSMSLKAALTLPRRPFLHCSTVIPVVVLTLKFTMHLFKLKDSWCFLPWMLFISWTSHHIRDGIRHGLWICPFGKTSPLPFWLYVIITSSLPHICSFVMYLTGTRQMMSSKHGVRIDV

SEQ ID NO: 21 - protein, 1337 aa, Homo sapiens
MDFTAQPKPATALCGVVSADGKIAYPPGVKEITDKITTDEMIKRLKMVVKTFMDMDQDSEDEKQQYLPLALHLASEFFLRNPNKDVRLLVACCLADIFRIYAPEAPYTSHDKLKDIFLFITRQLKGLEDTKSPQFNRYFYLLENLAWVKSYNICFELEDCNEIFIQLFRTLFSVINNSHNKKVQMHMLDLMSSIIMEGDGVTQELLDSILINLIPAHKNLNKQSFDLAKVLLKRTVQTIEACIANFFNQVLVLGRSSVSDLSEHVFDLIQELFAIDPHLLLSVMPQLEFKLKSNDGEERLAVVRLLAKLFGSKDSDLATQNRPLWQCFLGRFNDIHVPVRLESVKFASHCLMNHPDLAKDLTEYLKVRSHDPEEAIRHDVIVTIITAAKRDLALVNDQLLGFVRERTLDKRWRVRKEAMMGLAQLYKKYCLHGEAGKEAAEKVSWIKDKLLHIYYQNSIDDKLLVEKIFAQYLVPHNLETEERMKCLYYLYASLDPNAVKALNEMWKCQNMLRSHVRELLDLHKQPTSEANCSAMFGKLMTIAKNLPDPGKAQDFVKKFNQVLGDDEKLRSQLELLISPTCSCKQADICVREIARKLANPKQPTNPFLEMVKFLLERIAPVHIDSEAISALVKLMNKSIEGTADDEEEGVSPDTAIRSGLELLKVLSFTHPTSFHSAETYESLLQCLRMEDDKVAEAAIQIFRNTGHKIETDLPQIRSTLIPILHQKAKRGTPHQAKQAVHCIHAIFTNKEVQLAQIFEPLSRSLNADVPEQLITPLVSLGHISMLAPDQFASPMKSVVANFIVKDLLMNDRSTGEKNGKLWSPDEEVSPEVLAKVQAIKLLVRWLLGMKNNQSKSANSTLRLLSAMLVSEGDLTEQKRISKSDMSRLRLAAGSAIMKLAQEPCYHEIITPEQFQLCALVINDECYQVRQIFAQKLHKALVKLLLPLEYMAIFALCAKDPVKERRAHARQCLLKNISIRREYIKQNPMATEKLLSLLPEYVVPYMIHLLAHDPDFTRSQDVDQLRDIKECLWFMLEVLMTKNENNSHAFMKKMAENIKLTRDAQSPDESKTNEKLYTVCDVALCVINSKSALCNADSPKDPVLPMKFFTQPEKDFCNDKSYISEETRVLLLTGKPKPAGVLGAVNKPLSATGRKPYVRSTGTETGSNINVNSELNPSTGNRSREQSSEAAETGVSENEENPVRIISVTPVKNIDPVKNKEINSDQATQGNISSDRGKKRTVTAAGAENIQQKTDEKVDESGPPAPSKPRRGRRPKSESQGNATKNDDLNKPINKGRKRAAVGQESPGGLEAGNAKAPKLQDLAKKAAPAERQIDLQR

SEQ ID NO: 22 - protein, 634 aa, Homo sapiens
MAEEQEFTQLCKLPAQPSHPHCVNNTYRSAQHSQALLRGLLALRDSGILFDVVLVVEGRHIEAHRILLAASCDYFRGMFAGGLKEMEQEEVLIHGVSYNAMCQILHFIYTSELELSLSNVQETLVAACQLQIPEIIHFCCDFLMSWVDEENILDVYRLAELFDLSRLTEQLDTYILKNFVAFSRTDKYRQLPLEKVYSLLSSNRLEVSCETEVYEGALLYHYSLEQVQADQISLHEPPKLLETVRFPLMEAEVLQRLHDKLDPSPLRDTVASALMYHRNESLQPSLQSPQTELRSDFQCVVGFGGIHSTPSTVLSDQAKYLNPLLGEWKHFTASLAPRMSNQGIAVLNNFVYLIGGDNNVQGFRAESRCWRYDPRHNRWFQIQSLQQEHADLSVCVVGRYIYAVAGRDYHNDLNAVERYDPATNSWAYVAPLKREVYAHAGATLEGKMYITCGRRGEDYLKETHCYDPGSNTWHTLADGPVRRAWHGMATLLNKLYVIGGSNNDAGYRRDVHQVACYSCTSGQWSSVCPLPAGHGEPGIAVLDNRIYVLGGRSHNRGSRTGYVHIYDVEKDCWEEGPQLDNSISGLAACVLTLPRSLLLEPPRGTPDRSQADPDFASEVMSVSDWEEFDNSSED

SEQ ID NO: 23 - protein, 2268 aa, Homo sapiens
MKQLQPQPPPKMGDFYDPEHPTPEEEENEAKIENVQKTGFIKGPMFKGVASSRFLPKGTKTKVNLEEQGRQKVSFSFSLTKKTLQNRFLTALGNEKQSDTPNPPAVPLQVDSTPKMKMEIGDTLSTAEESSPPKSRVELGKIHFKKHLLHVTSRPLLATTTAVASPPTHAAPLPAVIAESTTVDSPPSSPPPPPPPAQATTLSSPAPVTEPVALPHTPITVLMAAPVPLPVDVAVRSLKEPPIIIVPESLEADTKQDTISNSLEEHVTQILNEQADISSKKEDSHIGKDEEIPDSSKISLSCKKTGSKKKSSQSEGIFLGSESDEDSVRTSSSQRSHDLKFSASIEKERDFKKSSAPLKSEDLGKPSRSKTDRDDKYFSYSKLERDTRYVSSRCRSERERRRSRSHSRSERGSRTNLSYSRSERSHYYDSDRRYHRSSPYRERTRYSRPYTDNRARESSDSEEEYKKTYSRRTSSHSSSYRDLRTSSYSKSDRDCKTETSYLEMERRGKYSSKLERESKRTSENEAIKRCCSPPNELGFRRGSSYSKHDSSASRYKSTLSKPIPKSDKFKNSFCCTELNEEIKQSHSFSLQTPCSKGSELRMINKNPEREKAGSPAPSNRLNDSPTLKKLDELPIFKSEFITHDSHDSIKELDSLSKVKNDQLRSFCPIELNINGSPGAESDLATFCTSKTDAVLMTSDDSVTGSELSPLVKACMLSSNGFQNISRCKEKDLDDTCMLHKKSESPFRETEPLVSPHQDKLMSMPVMTVDYSKTVVKEPVDTRVSCCKTKDSDIYCTLNDSNPSLCNSEAENIEPSVMKISSNSFMNVHLESKPVICDSRNLTDHSKFACEEYKQSIGSTSSASVNHFDDLYQPIGSSGIASSLQSLPPGIKVDSLTLLKCGENTSPVLDAVLKSKKSSEFLKHAGKETIVEVGSDLPDSGKGFASRENRRNNGLSGKCLQEAQEEGNSILPERRGRPEISLDERGEGGHVHTSDDSEVVFSSCDLNLTMEDSDGVTYALKCDSSGHAPEIVSTVHEDYSGSSESSNDESDSEDTDSDDSSIPRNRLQSVVVVPKNSTLPMEETSPCSSRSSQSYRHYSDHWEDERLESRRHLYEEKFESIASKACPQTDKFFLHKGTEKNPEISFTQSSRKQIDNRLPELSHPQSDGVDSTSHTDVKSDPLGHPNSEETVKAKIPSRQQEELPIYSSDFEDVPNKSWQQTTFQNRPDSRLGKTELSFSSSCEIPHVDGLHSSEELRNLGWDFSQEKPSTTYQQPDSSYGACGGHKYQQNAEQYGGTRDYWQGNGYWDPRSGRPPGTGVVYDRTQGQVPDSLTDDREEEENWDQQDGSHFSDQSDKFLLSLQKDKGSVQAPEISSNSIKDTLAVNEKKDFSKNLEKNDIKDRGPLKKRRQEIESDSESDGELQDRKKVRVEVEQGETSVPPGSALVGPSCVMDDFRDPQRWKECAKQGKMPCYFDLIEENVYLTERKKNKSHRDIKRMQCECTPLSKDERAQGEIACGEDCLNRLLMIECSSRCPNGDYCSNRRFQRKQHADVEVILTEKKGWGLRAAKDLPSNTFVLEYCGEVLDHKEFKARVKEYARNKNIHYYFMALKNDEIIDATQKGNCSRFMNHSCEPNCETQKWTVNGQLRVGFFTTKLVPSGSELTFDYQFQRYGKEAQKCFCGSANCRGYLGGENRVSIRAAGGKMKKERSRKKDSVDGELEALMENGEGLSDKNQVLSLSRLMVRIETLEQKLTCLELIQNTHSQSCLKSFLERHGLSLLWIWMAELGDGRESNQKLQEEIIKTLEHLPIPTKNMLEESKVLPIIQRWSQTKTAVPPLSEGDGYSSENTSRAHTPLNTPDPSTKLSTEADTDTPKKLMFRRLKIISENSMDSAISDATSELEGKDGKEDLDQLENVPVEEEEELQSQQLLPQQLPECKVDSETNIEASKLPTSEPEADAEIEPKESNGTKLEEPINEETPSQDEEEGVSDVESERSQEQPDKTVDISDLATKLLDSWKDLKEVYRIPKKSQTEKENTTTERGRDAVGFRDQTPAPKTPNRSRERDPDKQTQNKEKRKRRSSLSPPSSAYERGTKRPDDRYDTPTSKKKVRIKDRNKLSTEERRKLFEQEVAQREAQKQQQQMQNLGMTSPLPYDSLGYNAPHHPFAGYPPGYPMQAYVDPSNPNAGKVLLPTPSMDPVCSPAPYDHAQPLVGHSTEPLSAPPPVPVVPHVAAPVEVSSSQYVAQSDGVVHQDSSVAVLPVPAPGPVQGQ

SEQ ID NO: 24 - protein, 215 aa, Homo sapiens; identical to SEQ ID NO: 20

SEQ ID NO: 25 - protein, 840 aa, Homo sapiens
MNGEYRGRGFGRGRFQSWKRGRGGGNFSGKWREREHRPDLSKTTGKRTSEQTPQFLLSTKTPQSMQSTLDRFIPYKGWKLYFSEVYSDSSPLIEKIQAFEKFFTRHIDLYDKDEIERKGSILVDFKELTEGGEVTNLIPDIATELRDAPEKTLACMGLAIHQVLTKDLERHAAELQAQEGLSNDGETMVNVPHIHARVYNYEPLTQLKNVRANYYGKYIALRGTVVRVSNIKPLCTKMAFLCAACGEIQSFPLPDGKYSLPTKCPVPVCRGRSFTALRSSPLTVTMDWQSIKIQELMSDDQREAGRIPRTIECELVHDLVDSCVPGDTVTITGIVKVSNAEEGSRNKNDKCMFLLYIEANSISNSKGQKTKSSEDGCKHGMLMEFSLKDLYAIQEIQAEENLFKLIVNSLCPVIFGHELVKAGLALALFGGSQKYADDKNRIPIRGDPHILVVGDPGLGKSQMLQAACNVAPRGVYVCGNTTTTSGLTVTLSKDSSSGDFALEAGALVLGDQGICGIDEFDKMGNQHQALLEAMEQQSISLAKAGVVCSLPARTSIIAAANPVGGHYNKAKTVSENLKMGSALLSRFDLVFILLDTPNEHHDHLLSEHVIAIRAGKQRTISSATVARMNSQDSNTSVLEVVSEKPLSERLKVVPGETIDPIPHQLLRKYIGYARQYVYPRLSTEAARVLQDFYLELRKQSQRLNSSPITTRQLESLIRLTEARARLELREEATKEDAEDIVEIMKYSMLGTYSDEFGNLDFERSQHGSGMSNRSTAKRFISALNNVAERTYNNIFQFHQLRQIAKELNIQVADFENFIGSLNDQGYLLKKGPKVYQLQTM

SEQ ID NO: 26 - protein, 261 aa, Homo sapiens
MVVLSVPAEVTVILLDIEGTTTPIAFVKDILFPYIEENVKEYLQTHWEEEECQQDVSLLRKQAEEDAHLDGAVPIPAASGNGVDDLQQMIQAVVDNVCWQMSLDRKTTALKQLQGHMWRAAFTAGRMKAEFFADVVPAVRKWREAGMKVYIYSSGSVEAQKLLFGHSTEGDILELVDGHFDTKIGHKVESESYRKIADSIGCSTNNILFLTDVTREASAAEEADVHVAVVVRPGNAGLTDDEKTYYSLITSFSELYLPSST

SEQ ID NO: 27 - protein, 788 aa, Homo sapiens
MAMESTATAAVAAELVSADKIEDVPAPSTSADKVESLDVDSEAKKLLGLGQKHLVMGDIPAAVNAFQEAASLLGKKYGETANECGEAFFFYGKSLLELARMENGVLGNALEGVHVEEEEGEKTEDESLVENNDNIDEEAREELREQVYDAMGEKEEAKKTEDKSLAKPETDKEQDSEMEKGGREDMDISKSAEEPQEKVDLTLDWLTETSEEAKGGAAPEGPNEAEVTSGKPEQEVPDAEEEKSVSGTDVQEECREKGGQEKQGEVIVSIEEKPKEVSEEQPVVTLEKQGTAVEVEAESLDPTVKPVDVGGDEPEEKVVTSENEAGKAVLEQLVGQEVPPAEESPEVTTEAAEASAVEAGSEVSEKPGQEAPVLPKDGAVNGPSVVGDQTPIEPQTSIERLTETKDGSGLEEKVRAKLVPSQEETKLSVEESEAAGDGVDTKVAQGATEKSPEDKVQIAANEETQEREEQMKEGEETEGSEEDDKENDKTEEMPNDSVLENKSLQENEEEEIGNLELAWDMLDLAKIIFKRQETKEAQLYAAQAHLKLGEVSVESENYVQAVEEFQSCLNLQEQYLEAHDRLLAETHYQLGLAYGYNSQYDEAVAQFSKSIEVIENRMAVLNEQVKEAEGSSAEYKKEIEELKELLPEIREKIEDAKESQRSGNVAELALKATLVESSTSGFTPGGGGSSVSMIASRKPTDGASSSNCVTDISHLVRKKRKPEEESPRKDDAKKAKQEPEVNGGSGDAVPSGNEVSENMEEEAENQAESRAAVEGTVEAGATVESTAC

SEQ ID NO: 28 - protein, 609 aa, Homo sapiens [sequence omitted]

SEQ ID NO: 29 - protein, 609 aa, Homo sapiens [sequence omitted]

SEQ ID NO: 30 - protein, 453 aa, Homo sapiens
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

SEQ ID NO: 31 - protein, 453 aa, Homo sapiens; identical to SEQ ID NO: 30

SEQ ID NO: 32 - protein, 644 aa, Homo sapiens
MSRQFSSRSGYRSGGGFSSGSAGIINYQRRTTSSSTRRSGGGGGRFSSCGGGGGSFGAGGGFGSRSLVNLGGSKSISISVARGGGRGSGFGGGYGGGGFGGGGFGGGGFGGGGIGGGGFGGFGSGGGGFGGGGFGGGGYGGGYGPVCPPGGIQEVTINQSLLQPLNVEIDPEIQKVKSREREQIKSLNNQFASFIDKVRFLEQQNQVLQTKWELLQQVDTSTRTHNLEPYFESFINNLRRRVDQLKSDQSRLDSELKNMQDMVEDYRNKYEDEINKRTNAENEFVTIKKDVDGAYMTKVDLQAKLDNLQQEIDFLTALYQAELSQMQTQISETNVILSMDNNRSLDLDSIIAEVKAQYEDIAQKSKAEAESLYQSKYEELQITAGRHGDSVRNSKIEISELNRVIQRLRSEIDNVKKQISNLQQSISDAEQRGENALKDAKNKLNDLEDALQQAKEDLARLLRDYQELMNTKLALDLEIATYRTLLEGEESRMSGECAPNVSVSVSTSHTTISGGGSRGGGGGGYGSGGSSYGSGGGSYGSGGGGGGGRGSYGSGGSSYGSGGGSYGSGGGGGGHGSYGSGSSSGGYRGGSGGGGGGSSGGRGSGGGSSGGSIGGRGSSSGGVKSSGGSSSVKFVSTTYSGVTR

SEQ ID NO: 33 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 34 - protein, 30 aa, Homo sapiens
QAHGRCSAGAQFVFCRRSAGAACTQQALSR

SEQ ID NO: 35 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 36 - protein, 491 aa, Homo sapiens
MKRMVSWSFHKLKTMKHLLLLLLCVFLVKSQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 37 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 38 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 39 - protein, 609 aa, Homo sapiens [sequence omitted]

SEQ ID NO: 40 - protein, 42 aa, Homo sapiens
GQPREPQVYTLPPSRDELTKGFYPSDIAVEWESNGQPENNYK

SEQ ID NO: 41 - protein, 609 aa, Homo sapiens [sequence omitted]

SEQ ID NO: 42 - protein, 491 aa, Homo sapiens; identical to SEQ ID NO: 36

SEQ ID NO: 43 - protein, 491 aa, Homo sapiens; identical to SEQ ID NO: 36

SEQ ID NO: 44 - protein, 52 aa, Homo sapiens
GQPREPQVYTLPPSRDELTKNQVSLTCLVKGFYPSDIAVEWESNGQPENNYK

SEQ ID NO: 45 - protein, 453 aa, Homo sapiens; identical to SEQ ID NO: 30

SEQ ID NO: 46 - protein, 525 aa, Homo sapiens
MKALIAALLLITLQYSCAVSPTDCSAVEPEAEKALDLINKRRRDGYLFQLLRIADAHLDRVENTTVYYLVLDVQESDCSVLSRKYWNDCEPPDSRRPSEIVIGQCKVIATRHSHESQDLRVIDFNCTTSSVSSALANTKDSPVLIDFFEDTERYRKQANKALEKYKEENDDFASFRVDRIERVARVRGGEGTGYFVDFSVRNCPRHHFPRHPNVFGFCRADLFYDVEALDLESPKNLVINCEVFDPQEHENINGVPPHLGHPFHWGGHERSSTTKPPFKPHGSRDHHHPHKPHEHGPPPPPDERDHSHGPPLPQGPPPLLPMSCSSCQHATFGTNGAQRHSHNNNSSDLHPHKHHSHEQHPHGHHPHAHHPHEHDTHRQHPHGHHPHGHHPHGHHPHGHHPHGHHPHCHDFQDYGPCDPPPHNQGHCCHGHGPPPGHLRRRGPGKGPRPFHCRQIGSVYRLPPLRKGEVLPLPEANFPSFPLPHHKHPLKPDNQPFPQSVSESCPGKFKSGFPQVSMFFTHTFPK

SEQ ID NO: 47 - protein, 525 aa, Homo sapiens; identical to SEQ ID NO: 46

SEQ ID NO: 48 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 49 - protein, 377 aa, Homo sapiens
MCDDEETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVLSGGTTMYPGIADRMQKEITALAPSTMKIKIIAPPERKYSVWIGGSILASLSTFQQMWISKQEYDEAGPSIVHRKCF

SEQ ID NO: 50 - protein, 267 aa, Homo sapiens; identical to SEQ ID NO: 37

SEQ ID NO: 51 - protein, 377 aa, Homo sapiens; identical to SEQ ID NO: 49

SEQ ID NO: 52 - protein, 375 aa, Homo sapiens
MEEEIAALVIDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 53 - protein, 471 aa, Homo sapiens [sequence omitted]

SEQ ID NO: 54 - protein, 571 aa, Homo sapiens [sequence omitted]